(19) United States
(12) Patent Application Publication
(10) Pub. No.: US 2026/0364215 A1
(43) Pub. Date: Nov. 19, 2026

(54) Radiopharmaceutical Compositions Targeting Ephrin Type-A Receptor 2 and Uses Thereof

(71) Applicant: PeptiDream Inc., Kawasaki-shi (JP)

(72) Inventors: Zaid Amso, El Cajon, CA (US); Rongjun He, San Diego, CA (US); Derek Cole, San Diego, CA (US); Alain Noncovich, San Diego, CA (US); Takeru Ehara, Kanagawa (JP); Masaki Ohuchi, Kanagawa (JP); Takayuki Nagasawa, Kanagawa (JP); Shunichi Nakano, Kanagawa (JP); Kouki Morimoto, Kanagawa (JP); Makoto Jitsuoka, Kanagawa (JP); Sora Enya, Kanagawa (JP); Takanori Aoki, Kanagawa (JP); Hayato Yanagida, Kanagawa (JP)

(73) Assignee: RayzeBio, Inc., San Diego, CA (US)

(21) Appl. No.: 19/112,124

(22) PCT Filed: Sep. 28, 2023
(86) PCT No.: PCT/US2023/075451

§ 371 Date: Mar. 14, 2025

Related U.S. Application Data
(60) Provisional application No. 63/411,307, filed on Sep. 29, 2022.
(60) Provisional application No. 63/411,380, filed on Sep. 29, 2022.

(51) Int. Cl. A61K 51/08; A61P 35/00; C07K 7/64

(52) U.S. Cl. CPC A61K 51/088; A61P 35/00; C07K 7/64

(57) ABSTRACT

Provided herein are radiopharmaceutical conjugate compositions targeting EphA2 and uses thereof. In one aspect, provided herein are conjugates that comprise a peptide with avidity to EphA2 and a metal chelator configured to bind with a radionuclide. The conjugates described herein can further comprise a linker connecting the chelator and the peptide. The conjugates described herein can further comprise a radionuclide. In another aspect, provided herein are conjugates that comprise a peptide with avidity to EphA2 and a covalently bound radionuclide. The conjugates described herein can further comprise a linker connecting the radionuclide and the peptide. Further provided herein are methods of treating cancer by administering the described conjugates and compositions.

### CROSS REFERENCE TO RELATED APPLICATIONS

This application claims the benefit of U.S. Provisional Application No. 63/411,307, filed on Sep. 29, 2022, and U.S. Provisional Application No. 63/411,380, filed on Sep. 29, 2022, each of which is incorporated herein by reference in its entirety.

### JOINT RESEARCH AGREEMENTS

Subject matter disclosed herein was developed, and the claimed invention was made by, or on behalf of, one or more parties to a Joint Research Agreement (JRA), within the meaning of 35 U.S.C. § 100(h) and 37 C.F.R. § 1.9(e), that was in effect on or before the effective filing date of the claimed invention. Said one or more parties to the JRA consist of PeptiDream, Inc. (Kanagawa, Japan) and RayzeBio, Inc. (San Diego, CA, U.S.A.). The claimed invention was made as a result of activities undertaken within the scope of said Joint Research Agreement.

### SEQUENCE LISTING

The instant application contains a Sequence Listing which has been submitted electronically in XML format and is hereby incorporated by reference in its entirety. Said XML copy, created on Sep. 28, 2023, is named 59541-729_601_SL.xml and is 2,096,382 bytes in size.

### BACKGROUND

In the United States, cancer is the leading cause of death for those under 65 years of age, and it accounted for about 210% of all death in 2018. Traditional radiotherapies such as external beam radiation therapy have been used for decades as a standard-of-care treatment for diagnosed cancer patients. While some patients respond to external beam radiation therapy, many others do not. Further, metastasis and circulating tumor cells can spread and remain in the bloodstream or bodily fluids after standard-of-care treatment and lead to resistance to therapy. The presence of cancer cells in various parts of the body reduces the therapeutic efficacy of traditional radiotherapies. Accordingly, strategies for targeted radiotherapies are being developed, and there remains a need for targeted radiotherapies that have the desired affinity, stability, and exertion profile.

### SUMMARY

In one aspect, the present disclosure relates to a radiopharmaceutical conjugate comprising:
(a) a cyclic peptide that has avidity for ephrin type-A receptor 2 (EphA2), wherein the peptide comprises an amino acid sequence of Formula (I), or a pharmaceutically acceptable salt thereof,

X1-X2-X3-X4-X5-X6-X7-X8-X9-X10-X11-X12 Formula (I)
wherein,
   X1 is an amino acid;
   X2 is an amino acid comprising an aromatic ring, an N-methylated amino acid thereof, or a variant thereof,
   X3 is a hydrophilic amino acid (e.g. N, Q, Cit, K or a variant thereof), glycine (G), Alanine (A) or a variant thereof (e.g., da, 2-Aminoisobutyric acid (Aib));
   X4 is a hydrophobic amino acid (e.g., leucine (L)), a hydrophilic amino acid (e.g., citrulline (Cit)), or a variant thereof;
   X5 is a hydrophilic amino acid, or a variant thereof,
   X6 is a hydrophilic amino acid, an amino acid comprising an aromatic ring, or an N-methylated amino acid thereof,
   X7 is an amino acid comprising an aromatic ring (e.g., W, F, or a variant thereof);
   X8 is a hydrophobic amino acid, a hydrophilic amino acid, an N-methylated amino acid, or a variant thereof,
   X9 is an amino acid comprising an aromatic ring (e.g., W or a variant thereof);
   X10 is absent or a hydrophilic amino acid (e.g., Threonine (T) or a variant thereof);
   X11 is absent or a hydrophilic amino acid; and
   X12 is cysteine (C) or a variant thereof, and
(b)(i) a metal chelator configured to bind with a radionuclide, wherein the metal chelator is conjugated to the peptide; or (ii) a covalent radionuclide (or, a radionuclide covalently bound to the cyclic peptide). In some embodiments, the radiopharmaceutical conjugate comprises a metal chelator configured to bind with a radionuclide, wherein the metal chelator is conjugated to the peptide. In some embodiments, the radiopharmaceutical conjugate comprises a covalent radionuclide. In some embodiments, the radiopharmaceutical conjugate comprises a radionuclide covalently bound to the peptide. In some embodiments, the peptide is a cyclic peptide. In some embodiments, the peptide is a monocyclic peptide.

In one aspect, the present disclosure relates to a radiopharmaceutical conjugate comprising:
(a) a cyclic peptide that has avidity for ephrin type-A receptor 2 (EphA2), wherein the peptide comprises an amino acid sequence including deletion, substitution, and/or addition of one or several (e.g., 1-6) amino acids in the amino acid sequence of SEQ ID NO: 1:

(SEQ ID NO: 1)
da-MeF-N-L-Hgl-MeF-W1Me-V-W1Me-T-E-C

or a pharmaceutically acceptable salt thereof,
wherein the cyclic peptide consists of 10 or 12 amino acid residues; and
(b)(i) a metal chelator configured to bind with a radionuclide, wherein the metal chelator is conjugated to the peptide; or (ii) a covalent radionuclide. In some embodiments, the radiopharmaceutical conjugate comprises a metal chelator configured to bind with a radionuclide, wherein the metal chelator is conjugated to the peptide. In some embodiments, the radiopharmaceutical conjugate comprises a covalent radionuclide. In some embodiments, the radiopharmaceutical conjugate comprises a radionuclide covalently bound to the peptide.

In some embodiments, the radiopharmaceutical conjugate further comprises a radionuclide bound to the metal chelator. In some embodiments, the radionuclide is an alpha particle-emitting radionuclide. In some embodiments, the alpha particle-emitting radionuclide is selected from Ac-225, Bi-213, Bi-209, Tb-149, Ra-223, Th-227, Fr-223, Gd-148, Th-229, Pb-212, and Po-213. In some embodiments, the alpha particle-emitting radionuclide is Ac-225. In some embodiments, the radionuclide is a beta particle-emitting radionuclide. In some embodiments, the beta particle-emitting radionuclide is Cu-67, Lu-177, Y-90, Rh-105, Yb-175, Tm-167, Pm-153, Sm-153, or In-111. In some embodiments, the beta particle-emitting radionuclide is Lu-177. In some embodiments, the radionuclide is a gamma particle-emitting radionuclide. In some embodiments, the gamma particle-emitting radionuclide is indium-111 or tin-117m. In some embodiments, the radionuclide is a positron-emitting radionuclide. In some embodiments, the positron-emitting radionuclide is Ga-68, Cu-62, Cu-64, Zr-89, Tb-152.

In some embodiments, the metal chelator comprises DOTA, DOTA-GA, pBn-DOTA, pBn-SCN-DOTA, NH2-DOTA, NH2-DOTA-GA, p-NCS-Bn-DOTA-GA, p-NH2-Bn-oxo-DO3A, p-SCN-Bn-oxo-DO3A, NOTA, NODA-GA, NH2-NODA-GA, p-NCS-Bn-NODA-GA, p-NH2-Bn-NOTA, p-SCN-Bn-NOTA, NCS-MP-NODA, NH2-MPAA-NODA, PCTA, p-NH2-Bn-PCTA, p-SCN-Bn-PCTA, p-SCN-Bn-HEHA, H2-MACROPA-NCS, H1-MACROPA, H2-MACROPA-NH2, H4-OCTAPA, tetra-(S, S, S, S)-Me-DOTA, tetra-(S, S, S, S)-Et-DOTA, tetra-(S, S, S, S)-iBu-DOTA, or maleimide-nBu-DOTA. In some embodiments, the metal chelator has a structure of

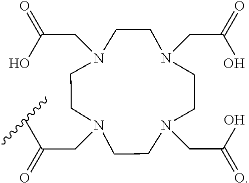 [SMILES: *C(=O)CN1CCN(CC(=O)O)CCN(CC(=O)O)CCN(CC(=O)O)CC1]

In some embodiments, the radiopharmaceutical conjugate further comprises a linker that connects the peptide with the metal chelator. In some embodiments, the linker covalently connects the peptide with the metal chelator.

In some embodiments, the radiopharmaceutical conjugate has a structure of:

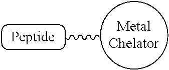

wherein
[symbol] represents the linker.

In some embodiments, the linker is attached to the peptide via a non-terminal amino acid residue of the peptide. In some embodiments, the linker is attached to the 5^{th} amino acid residue or X5. In some embodiments, the linker is attached to the 8^{th} amino acid residue or X8. In some embodiments, the linker is attached to the 11^{th} amino acid residue or X11.

In some embodiments, the radionuclide is covalently bound to an amino acid comprising an aromatic ring. In some embodiments, the radionuclide is ¹⁸F, ⁷⁴As, ⁷⁶Br, ¹²³I, ¹²⁴I, ¹²⁵I, ¹³¹I, or ²¹¹At. In some embodiments, the radionuclide is ¹⁸F, ¹²⁵I, ¹³¹I, or ²¹¹At. In some embodiments, the radionuclide is attached to X1, X2 or MeF, X6 or MeF, X7 or W1Me, or X9 or W1Me. In some embodiments, the radionuclide is attached to a tyrosine residue. In some embodiments, the radiopharmaceutical conjugate comprises a linker that connects the peptide with the radionuclide. In some embodiments, the linker covalently connects the peptide with the radionuclide.

In some embodiments, the radiopharmaceutical conjugate has a structure of

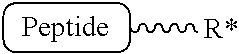

wherein
   [symbol] represents the linker; and
   R* represents the radionuclide.

In some embodiments, the linker is attached to the peptide via a non-terminal amino acid residue of the peptide. In some embodiments, the linker is attached to the 5^{th} amino acid residue or X5. In some embodiments, the linker is attached to the 8^{th} amino acid residue or X8. In some embodiments, the linker is attached to the 11^{th} amino acid residue or X11. In some embodiments, the linker comprises a residualizing agent. In some embodiments, the residualizing agent is chosen from is

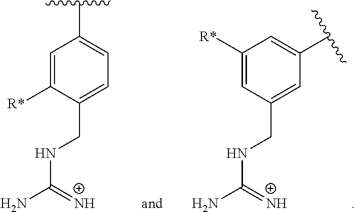

In some embodiments, [symbol] has a structure selected from:

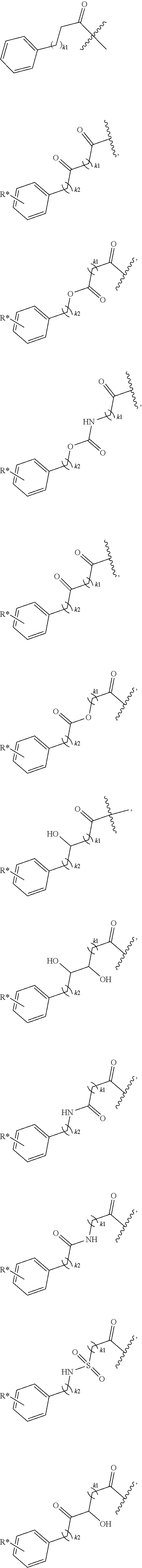

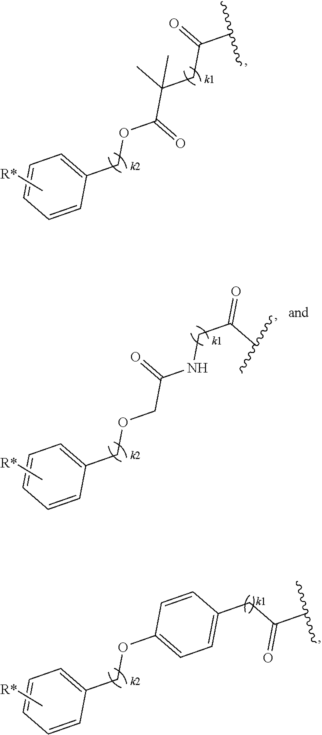

wherein each k1 and k2 is independently 0 or an integer selected from 1 to 10; and R* is the radionuclide.

In some embodiments, the peptide or the pharmaceutically accepted salt thereof has a cyclic structure, wherein the first amino acid (or X1) is covalently linked to the last amino acid (or X12). In some embodiments, the peptide or the pharmaceutically accepted salt thereof has a cyclic structure having an amino acid in the first residue X1 and a cysteine residue or a variant thereof, and wherein the amino acid in X1 and the cysteine residue or a variant thereof form a covalent bond. In some embodiments, the peptide consists of an amino acid sequence selected from SEQ ID NOs: 1-122, 159-163, and 165-171, and the peptide has a cyclic structure having a cysteine residue or a variant thereof at 12th residue, and wherein the amino acid X1 and the cysteine residue or a variant thereof at 12th residue form a covalent bond (e.g., by reacting a chloroacetyl group in the amino acid of X1 with the cysteine residue or a variant thereof). In some embodiments, the peptide can be cyclized by reacting a bromoacetyl group in the amino acid of X1 with the cysteine residue or a variant thereof. In some embodiments, the peptide consists of an amino acid sequence selected from SEQ ID NOs: 123-149 and 164, and the peptide has a cyclic structure having a cysteine residue or a variant thereof at 10th residue, and wherein the amino acid X1 and the cysteine residue or a variant thereof at 10th residue form a covalent bond.

In some embodiments, the radiopharmaceutical conjugate comprises a metal chelator configured to bind with a radionuclide, wherein the metal chelator is conjugated to the peptide. In some embodiments, the radiopharmaceutical conjugate comprises a covalently bound radionuclide. In some embodiments, the radiopharmaceutical conjugate comprises a radionuclide covalently bound to the peptide. In some embodiments, the peptide is a monocyclic peptide. In some embodiments, the peptide has an amino acid sequence according to Formula (I), or a pharmaceutically acceptable salt thereof. In some embodiments, the radiopharmaceutical conjugate comprises an amino acid sequence of Formula (I), or a pharmaceutically acceptable salt thereof,

X1-X2-X3-X4-X5-X6-X7-X8-X9-X10-X11-X12 Formula (I)
wherein,
   X1 is an amino acid;
   X2 is F, or a variant thereof that replaces the unsubstituted phenyl ring of F with
      (i) a phenyl ring substituted by 1 or 2 substituents each independently selected from —OH, —CN, and —C₁₋₃ alkyl, or
      (ii) a 6-membered heteroaryl ring optionally substituted by 1 or 2 substituents each independently selected from —OH, —CN, and —C₁₋₃ alkyl,
      wherein the F or the variant thereof is optionally N-methylated;
   X3 is a hydrophilic amino acid (e.g. N, Q, Cit, K or a variant thereof), G, Aib, Hgn, Ala, or a variant thereof (e.g., da);
   X4 is a hydrophobic amino acid (e.g., an amino acid having 4 or more carbon atoms in a side chain comprising a linear, branched, or cyclic carbon chain), and wherein X4 is optionally N-methylated (e.g., Cit or a variant thereof);
   X5 is an amino acid (e.g., a hydrophilic amino acid; Dab, Dap, R, E or a variant thereof; or an amino acid with a functional side chain);
   X6 is an N-methylated amino acid thereof;
   X7 is a W, Y, or a variant thereof (e.g., an amino acid having either a 6-membered aryl or heteroaryl, or a 9- or 10-membered bi-cyclic aryl or heteroaryl linked to the alpha-carbon through a carbon (e.g., a methylene group), wherein the 6-, 9-, and 10-membered heteroaryl has one heteroatom (e.g., N), and wherein the 6-, 9-, and 10-membered aryl or heteroaryl is optionally substituted by 1 or 2 substituents independently selected from —CH₃, -ethyl, —Cl, and —F);
   X8 is an amino acid with —H on the alpha-amino group;
   X9 is W or Y or a variant thereof, (e.g., W or a variant thereof);
   X10 is absent, or a polar amino acid (e.g., T or a variant thereof);
   X11 is absent, or an amino acid (e.g., a hydrophilic amino acid; Dab, Dap, R, E or a variant thereof, or an amino acid with a functional side chain); and
   X12 is C or a variant thereof.

In some embodiments, the radiopharmaceutical conjugate comprises an amino acid sequence of Formula (Ia), or a pharmaceutically acceptable salt thereof,

X1-X2-X3-X4-X5-X6-X7-X8-X9-X12 Formula (Ia)
wherein,
   X1 is an amino acid (e.g., D-amino acid);
   X2 is an amino acid comprising an aromatic ring, an N-methylated amino acid thereof, or a variant thereof,
   X3 is a hydrophilic amino acid (e.g., N, Q, Cit, K or a variant thereof), G, A, or a variant thereof (e.g., da, Aib);
   X4 is a hydrophobic amino acid, or a hydrophilic amino acid (e.g., Cit or a variant thereof);
   X5 is a hydrophilic amino acid (e.g., Dab, Dap, R, E, Q, D, K), or a variant thereof);
   X6 is a hydrophilic amino acid, an amino acid comprising an aromatic ring (e.g., W, or F, or a variant thereof), or an N-methylated amino acid thereof,
   X7 is an amino acid comprising an aromatic ring (e.g., W, F, or a variant thereof);
   X8 is a hydrophobic amino acid, a hydrophilic amino acid, or an N-methylated amino acid;
   X9 is an amino acid comprising an aromatic ring (e.g., W, F or a variant thereof); and
   X12 is C or a variant thereof.

In some embodiments, the radiopharmaceutical conjugate comprises an amino acid sequence according to Formula (I), or a pharmaceutically acceptable salt thereof,

X1-X2-X3-X4-X5-X6-X7-X8-X9-X10-X11-X12 Formula (I)
wherein,
   X1 is an amino acid (e.g., D-amino acid);
   X2 is an amino acid comprising an aromatic ring, an N-methylated amino acid thereof, or a variant thereof,
   X3 is a hydrophilic amino acid (e.g., N, Q, Cit, K or a variant thereof), G, A, or a variant thereof (e.g., da, Aib);
   X4 is a hydrophobic amino acid, or a hydrophilic amino acid (e.g., Cit or a variant thereof);
   X5 is a hydrophilic amino acid (e.g., Dab, Dap, R, E, Q, D, K), or a variant thereof);
   X6 is a hydrophilic amino acid, an amino acid comprising an aromatic ring (e.g., W, or F, or a variant thereof), or an N-methylated amino acid thereof,
   X7 is an amino acid comprising an aromatic ring (e.g., W, F, or a variant thereof);
   X8 is a hydrophobic amino acid, a hydrophilic amino acid, or an N-methylated amino acid;
   X9 is an amino acid comprising an aromatic ring (e.g., W, F or a variant thereof);
   X10 is a hydrophilic amino acid (e.g., T, S, N, Q, K, Cit, or a variant thereof);
   X11 is a hydrophilic amino acid; and
   X12 is C or a variant thereof.

In some embodiments, the radiopharmaceutical conjugate comprises an amino acid sequence according to Formula (I), or a pharmaceutically acceptable salt thereof,

X1-X2-X3-X4-X5-X6-X7-X8-X9-X10-X11-X12 Formula (I)
wherein,
   X1 is da, df3CON, dkCOpipzaa, dahp, dDab-NH₂-Ph3-SO₂F, dDap-NH₂-Ph3-SO₂F, dDap-NH₂-Ph4-SO₂F, dCit, Aib, G, Norvaline, Norleucine, d4PyCON, or dhAla;
   X2 is MeF, Me3Py, MeF3CON, MeF3F, Me4Py, or MeY(Me);
   X3 is absent, N, Q, Cit, G, Aib, Hgn, hCit, norCit, LysAc, OrnAc, Ala, or da;
   X4 is L, Cbg, Chg, Cba, Cha, Ahx, Dahp, Cit, I, V, Norleucine, or Norvaline;
   X5 is Hgl, Hgn, Dab, Dap, DabAc, DapAc, R, hArg, E, or D;
   X6 is absent, MeF, MeE, Me3Py, Me4Py, MeF4F, MeF4F, MeF4C, or MeY;
   X7 is W1Me, W1Me7Cl, W1Me7N, W, F, 7-AzaTrp, W7Me, W1Et, W1Me7Br, W1Me7OMe, or W1Me6O7Cl;
   X8 is V, KCOpipzaa, N, Cit, Qglucamine, hCit, K, KAc, Aib, Alb, DapAc, OrnAc, A, T, alT, Norleucine, Norvaline, Hgl, E, Hgn, Q, I, or L;
   X9 is W1Me, W1Me7Cl, W1Me7N, F23dMe, W1Et, W7Me, W, F, or 7-AzaTrp;
   X10 is absent, T, Q, S, Hgn, Alpha-methylserine, hSer, hThr, N, OrnAc, LysAc, Cit, or hCit;
   X11 is absent, E, Hgn, R, hArg, Cit, hCit, Hgl, Orn, D, N, Q, DapAc, OrnAc, DabAc, norCit; and
   X12 is C, hCys, CdMe, C3RMe, C3SMe, Selenocysteine, dc, or Penicillamine.

In one aspect, the present disclosure relates to a radiopharmaceutical conjugate comprising:
(a) a cyclic peptide that has avidity for ephrin type-A receptor 2 (EphA2), wherein the peptide consists of a sequence of Formula (I),

X1-X2-X3-X4-X5-X6-X7-X8-X9-X10-X11-X12 Formula (I)

or a pharmaceutically acceptable salt thereof,
wherein
each of X1, X2, X3, X4, X5, X6, and X8 is independently an amino acid;
   X7 is W1Me or a variant thereof,
   X9 is W1Me or a variant thereof;
   each of X10 and X11 is independently absent or an amino acid; and
   X12 is cysteine (C) or a variant thereof;
   (b)(i) a metal chelator configured to bind with a radionuclide; or (ii) a covalent radionuclide (or, a radionuclide covalently bound to the cyclic peptide); and
   (c)(i) optionally, a linker that connects the peptide with the metal chelator; or (ii) optionally, a linker that connects the peptide with the covalent radionuclide. In some embodiments, the radiopharmaceutical conjugate comprises a metal chelator configured to bind with a radionuclide and optionally, a linker that connects the peptide with the metal chelator. In some embodiments, the radiopharmaceutical conjugate comprises a covalent radionuclide and optionally, a linker that connects the peptide with the covalent radionuclide. In some embodiments, the radiopharmaceutical conjugate comprises a radionuclide covalently bound to the peptide. In some embodiments, the radiopharmaceutical conjugate comprises a linker that connects the peptide with the covalent radionuclide.

In one aspect, the present disclosure relates to a radiopharmaceutical conjugate comprising:
(a) a cyclic peptide that has avidity for ephrin type-A receptor 2 (EphA2), wherein the peptide has an amino acid sequence of Formula (I),

X1-X2-X3-X4-X5-X6-X7-X8-X9-X10-X11-X12 Formula (I)
wherein,
   X1 is any D- or L-amino acid;
   X2 has a structure of

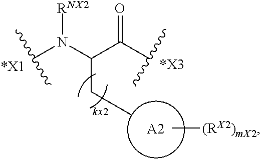

wherein
ring A2 is phenyl or a 6-membered heteroaryl (e.g., heteroaryl having 1 or 2 N);
R^{X2} is each independently halogen, —CN, —NO₂, —OH, —OR^{a}, —OC(═O)R^{a}, —OC(═O)OR^{b}, —OC(═O)NR^{c}R^{d}, —SH, SF₅, —SW, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
kx2 is 0, 1, 2, or 3;
mx2 is 0, 1, 2, 3 or 4;
R^{NX2} is H, C₁-C₆alkyl, or C₁-C₆haloalkyl;
*X1 indicates the point of attachment to X1; and,
*X3 indicates the point of attachment to X3;
X3 has a structure of

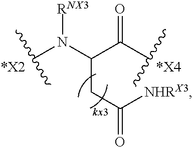

wherein
kx3 is 0, 1, 2, or 3;
R^{NX3} is H, C₁-C₆alkyl, or C₁-C₆haloalkyl;
R^{X3} is H, C₁-C₆alkyl, C₁-C₆haloalkyl, C₁-C₆hydroxyalkyl, C₁-C₆aminoalkyl, or C₁-C₆heteroalkyl;
*X2 indicates the point of attachment to X2; and,
*X4 indicates the point of attachment to X4;
X4 is a hydrophobic amino acid (e.g., amino acid having 4 or more carbon atoms in a side chain comprising a linear, branched, or cyclic carbon chain), and wherein X4 is optionally N-alkylated by a C₁₋₃ alkyl group;
X5 is a hydrophilic L-amino acid, such as an amino acid having a structure of

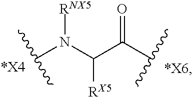

wherein:
R^{NX5} is H, —CN, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA};
R^{X5} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═NR^{b})NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA};
provided that at least one of R^{NX5} and R^{X5} comprises a moiety selected from —OH, —NH₂, and —NH— (e.g., —NH—C(═NH)—NH₂, —CO—NH₂, —NH₂, —COOH, —C(OH)—C₀₋₆ alkyl, —NH—CO—C₁₋₆ alkyl);
*X4 indicates the point of attachment to X4; and,
*X6 indicates the point of attachment to X6;
X6 is

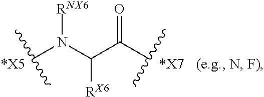

wherein
R^{NX6} is H, C₁-C₆alkyl, or C₁-C₆haloalkyl;
R^{X6} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═NR^{b})NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl or heteroaryl; wherein the alkyl, haloalkyl, hydroxyalkyl, aminoalkyl, heteroalkyl, cycloalkyl, heterocycloalkyl, aryl or heteroaryl is optionally and independently substituted with one or more R^{A};
*X5 indicates the point of attachment to X5; and,
*X7 indicates the point of attachment to X7;
X7 has a structure of

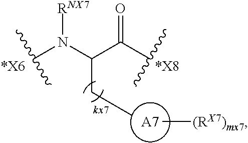

wherein
R^{NX7} is H, C₁-C₆alkyl, or C₁-C₆haloalkyl;
ring A7 is an aryl or heteroaryl;
R^{X7} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂-halogen, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{d}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{A};
kx7 is 0, 1, 2, or 3;
mx7 is 0, 1, 2, 3, 4 or 5;
X6 indicates the point of attachment to X6; and,
X8 indicates the point of attachment to X8;
X8 is an L-amino acid comprising an —H on the alpha-amino group;
X9 has a structure of

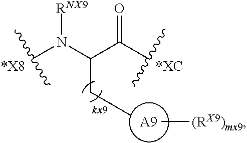

wherein
R^{NX9} is H, C₁-C₆alkyl, or C₁-C₆haloalkyl;
ring A9 is an aryl or heteroaryl;
R^{X9} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
kx9 is 0, 1, 2, or 3;
mx9 is 0, 1, 2, 3, 4, or 5;
*X8 indicates the point of attachment to X8; and,
*XC indicates the point of attachment to (i) X10 or (i) when X10 and X11 are absent, X12;
X10 is absent or an L-amino acid;
X11 is absent or an L-amino acid; provided that when X10 is absent, then X11 is also absent; and
X12 is an L-amino acid having a reactive thiol group, such as Cys and Cys variants;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R and R^{XA} is independently halogen, —CN, —OH, —OC₁-C₆alkyl, SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NR^{b}C(═NR^{b})NR^{c}R^{d}, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
(b)(i) a metal chelator configured to bind with a radionuclide; or (ii) a covalent radionuclide; and
(c)(i) optionally, a linker that connects the peptide with the metal chelator; or (ii) optionally, a linker that connects the peptide with the covalent radionuclide. In some embodiments, the radiopharmaceutical conjugate comprises a metal chelator configured to bind with a radionuclide and optionally, a linker that connects the peptide with the metal chelator. In some embodiments, the radiopharmaceutical conjugate comprises a covalent radionuclide and optionally, a linker that connects the peptide with the covalent radionuclide. In some embodiments, the radiopharmaceutical conjugate comprises a radionuclide covalently bound to the peptide. In some embodiments, the radiopharmaceutical conjugate comprises a linker that connects the peptide with the covalent radionuclide.

In some embodiments, X7 is W1Me; X8 is V; and X9 is W1Me.

In some embodiments, the radiopharmaceutical conjugate comprises an amino acid sequence according to Formula (I), or a pharmaceutically acceptable salt thereof,

X1-X2-X3-X4-X5-X6-X7-X8-X9-X10-X11-X12 Formula (I)
wherein,
   X1 is any amino acid
   X2 is an amino acid having an aromatic ring or a variant thereof
   X3 is N,
   X4 is a hydrophobic amino acid or a variant thereof,
   X5 is a hydrophilic amino acid or a variant thereof,
   X6 is a hydrophilic amino acid or amino acid having aromatic ring;
   X7 is W or a variant thereof,
   X8 is V or hydrophilic amino acid or a variant thereof,
   X9 is W or a variant thereof,
   X10 is T or a variant thereof,
   X11 is a hydrophilic amino acid;
   X12 is C or a variant thereof (such as C).

In some embodiments, the radiopharmaceutical conjugate comprises an amino acid sequence according to Formula (Ia), or a pharmaceutically acceptable salt thereof,

X1-X2-X3-X4-X5-X6-X7-X8-X9-X12 Formula (Ia)
wherein,
   X1 is any amino acid;
   X2 is an amino acid having an aromatic ring or a variant thereof,
   X3 is N or a variant thereof,
   X4 is a hydrophobic amino or a variant thereof,
   X5 is a hydrophilic amino acid or a variant thereof,
   X6 is a hydrophilic amino acid or amino acid having aromatic ring;
   X7 is W or a variant thereof,
   X8 is a hydrophilic amino acid or a variant thereof,
   X9 is W or a variant thereof, and
   X12 is C or a variant thereof.

In some embodiments, the peptide has a structure of Formula (I-1),

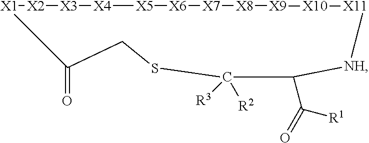

wherein
   R¹ is selected from the group consisting of NH₂ and OH;
   R² is selected from the group consisting of H or C₁₋₃ alkyl;
   R³ is selected from the group consisting of H or C₁₋₃ alkyl;
wherein X1 to X11 have the definitions described in Formula (I), and wherein the attachment point to the radionuclide or the linker is not shown.

In some embodiments, the peptide of Formula (I-1) has a structure of Formula (I-2),

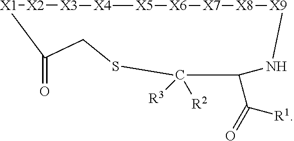

In some embodiments, the conjugate has a structure of Formula (III-1)

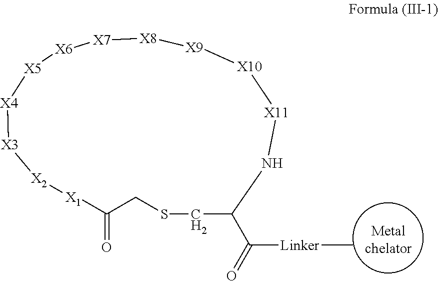

wherein X1 to X11 have the definitions described in Formula (I), and
wherein -Linker- represents the linker connecting the peptide and the metal chelator.

In some embodiments, the conjugate has a structure of Formula (III-1-RI)

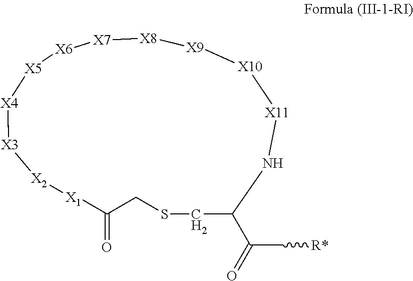

wherein X1 to X11 have the definitions described in Formula (I), and
wherein [symbol] represents the linker connecting the peptide and the radionuclide R*.

In some embodiments, the conjugate has a structure of Formula (III-2),

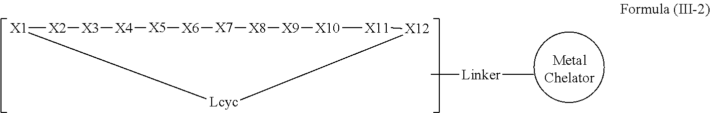

wherein
   Lcyc is a ring closing group that covalently connects X1 with X12;
   -Linker- represents the linker that connects the peptide and the metal chelator; and
wherein X1 to X12 have the definitions described in Formula (I).

In some embodiments, the conjugate has a structure of Formula (III-2-RI),

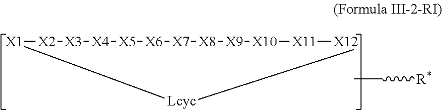

wherein
   Lcyc is a ring closing group that covalently connects X1 with X12;
   [symbol] represents the linker that connects the peptide and the radionuclide R*; and
wherein X1 to X12 have the definitions described in Formula (I).

In some embodiments, the peptide or the salt thereof comprises an amino acid sequence that is at least 90% identical to a sequence selected from SEQ ID NOs: 1-171. In some embodiments, the peptide or the salt thereof consists of an amino acid sequence selected from SEQ ID NOs: 1-171. In some embodiments, the peptide or salt thereof is not SEQ ID NO: 1. In some embodiments, the peptide or salt thereof does not comprise SEQ ID NO: 1. In some embodiments, the radiopharmaceutical conjugate is not SEQ ID NO: 282.

In some embodiments, the radiopharmaceutical conjugate comprises a peptide that interacts with a human EphA2 at one or more amino acid residues selected from Asp53, Met55, Asn57, Met59, Met66, Thr101, Arg103, Phe156, Glu157, Arg159, Val161, Val189, and Ala190. In some embodiments, the peptide interacts with a human EphA2 at Asp53 and Glu157. In some embodiments, the peptide is a peptide of Formula (I) and wherein, when the peptide is bound to the human EphA2, amino acid residue X7 is located less than 10 Å from the Phe156 of the human EphA2. In some embodiments, the peptide is a peptide of Formula (I) and wherein, when the peptide is bound to the human EphA2, amino acid residue X9 is located less than 10 Å from the Phe156 of the human EphA2. In some embodiments, the peptide is a peptide of Formula (I) and wherein, when the peptide is bound to the human EphA2, amino acid residue X8 is located less than 10 Å from the Phe156 of the human EphA2. In some embodiments, the human EphA2 comprises a sequence of SEQ ID NO: 276 or SEQ ID NO: 277.

In some embodiments, the conjugate is a compound of Tables 1, 2A, 2B, 2B-Lu, 2B-Lu-177, 2B-Ac-225, or 2C.

In one aspect, the present disclosure relates to a radiopharmaceutical conjugate, comprising:
(a) a peptide that has avidity for ephrin type-A receptor 2 (EphA2), wherein the peptide competes for binding to a human EphA2 with a peptide that has an amino acid sequence including deletion, substitution, and/or addition of one or several amino acids in the amino acid of SEQ ID NO:1:

(SEQ ID NO: 1)
da-MeF-N-L-Hgl-MeF-W1Me-V-W1Me-T-E-C

or a pharmaceutically acceptable salt thereof; and
(b)(i) a metal chelator configured to bind with a radionuclide, wherein the metal chelator is conjugated to the peptide; or (ii) a covalent radionuclide. In some embodiments, the radiopharmaceutical conjugate comprises a metal chelator configured to bind with a radionuclide, wherein the metal chelator is conjugated to the peptide. In some embodiments, the radiopharmaceutical conjugate comprises a covalent radionuclide. In some embodiments, the radiopharmaceutical conjugate comprises a radionuclide covalently bound to the peptide.

In one aspect, the present disclosure relates to a radiopharmaceutical conjugate, comprising:
(a) a peptide that has avidity for ephrin type-A receptor 2 (EphA2), wherein the peptide competes for binding to a human EphA2 with a peptide that has a structure of Formula (I), or a pharmaceutically acceptable salt thereof,

X1-X2-X3-X4-X5-X6-X7-X8-X9-X10-X11-X12 Formula (I)
wherein,
   X1 is an amino acid;
   X2 is an amino acid comprising an aromatic ring, an N-methylated amino acid thereof, or a variant thereof;
   X3 is a hydrophilic amino acid (e.g. N, Q, Cit, K or a variant thereof), glycine (G), Alanine (A) or a variant thereof (e.g., da, 2-Aminoisobutyric acid (Aib));
   X4 is a hydrophobic amino acid (e.g., leucine (L)), a hydrophilic amino acid (e.g., citrulline (Cit)), or a variant thereof;
   X5 is a hydrophilic amino acid, or a variant thereof;
   X6 is a hydrophilic amino acid, an amino acid comprising an aromatic ring, or an N-methylated amino acid thereof;
   X7 is an amino acid comprising an aromatic ring (e.g., W, F, or a variant thereof);
   X8 is a hydrophobic amino acid, a hydrophilic amino acid, an N-methylated amino acid, or a variant thereof;
   X9 is an amino acid comprising an aromatic ring (e.g., W or a variant thereof);
   X10 is absent or a hydrophilic amino acid (e.g., Threonine (T) or a variant thereof);
   X11 is absent or a hydrophilic amino acid; and
   X12 is cysteine (C) or a variant thereof; and
(b)(i) a metal chelator configured to bind with a radionuclide, wherein the metal chelator is conjugated to the peptide; or (ii) a covalent radionuclide. In some embodiments, the radiopharmaceutical conjugate comprises a metal chelator configured to bind with a radionuclide, wherein the metal chelator is conjugated to the peptide. In some embodiments, the radiopharmaceutical conjugate comprises a covalent radionuclide. In some embodiments, the radiopharmaceutical conjugate comprises a radionuclide covalently bound to the peptide.

In one aspect, the present disclosure relates to a radiopharmaceutical conjugate comprising:
(a) a cyclic peptide that has avidity for ephrin type-A receptor 2 (EphA2), wherein the peptide consists of a sequence of Formula (I),

X1-X2-X3-X4-X5-X6-X7-X8-X9-X10-X11-X12 Formula (I)
or a pharmaceutically acceptable salt thereof,
wherein
   each of X1, X2, X3, X4, X5, X6, and X8 is independently an amino acid;
   X7 is W1Me or a variant thereof;
   X9 is W1Me or a variant thereof;
   each of X10 and X11 is independently absent or an amino acid; and
   X12 is cysteine (C) or a variant thereof;
(b)(i) a metal chelator configured to bind with a radionuclide; or (ii) a covalent radionuclide; and
(c)(i) a linker that connects the peptide with the metal chelator; or (ii) a linker that connects the peptide with the a covalent radionuclide. In some embodiments, the radiopharmaceutical conjugate comprises a metal chelator configured to bind with a radionuclide. In some embodiments, the radiopharmaceutical conjugate comprises a linker that connects the peptide with the metal chelator. In some embodiments, the radiopharmaceutical conjugate comprises a covalent radionuclide. In some embodiments, the radiopharmaceutical conjugate comprises a linker that connects the peptide with the covalent radionuclide.

In one aspect, the present disclosure relates to a pharmaceutical composition comprising a radiopharmaceutical conjugate as described herein, and a pharmaceutically acceptable excipient or carrier.

In one aspect, the present disclosure relates to a radiolabeled human EphA2 protein, wherein the EphA2 protein is bound to a radiopharmaceutical conjugate as described herein.

In one aspect, the present disclosure relates to a method of treating a disease or disorder characterized by overexpression of EphA2, comprising administering to the subject a radiopharmaceutical conjugate as described herein, or a pharmaceutical composition thereof. In some embodiments, the disease or disorder is cancer.

In one aspect, the present disclosure relates to a method of diagnosing or imaging a cancer in a subject in need thereof, comprising administering to the subject a radiopharmaceutical conjugate as described herein, or a pharmaceutical composition thereof.

In one aspect, the present disclosure relates to a method of treating a cancer in a subject in need thereof, comprising administering to the subject a radiopharmaceutical conjugate as described herein, or a pharmaceutical composition thereof. In some embodiments, the cancer is selected from glioblastoma, prostate cancer, lung cancer, breast cancer, gastric cancer, ovarian cancer, bladder cancer, colon cancer, esophageal cancer, multiple myeloma and fibrosarcoma. In some embodiments, the cancer is non-small cell lung carcinomas (NSCLC). In some embodiments, the cancer is triple negative breast cancer. In some embodiments, the method comprises administering (i) a first radiopharmaceutical conjugate comprising a radionuclide configured for companion diagnostic (such as PET imaging) and (ii) a second radiopharmaceutical conjugate comprising a radionuclide selected from an alpha or beta-particle emitter, wherein the first and the second conjugate have the same structure except for the radionuclide. In some embodiments, the radionuclide of the first conjugate is selected from Lu-177, In-111, Ga-68, Cu-64, and Zr-89. In some embodiments, the radionuclide of the first conjugate is selected from ¹⁸F, ⁷⁴As, ⁷⁶Br, ¹²³I, ¹²⁴I, and ¹²⁵I. In some embodiments, the radionuclide of the second conjugate is selected from ¹³¹I and ²¹¹At.

In one aspect, disclosed herein is a pharmaceutical composition comprising a radiopharmaceutical conjugate or a salt thereof as described herein, and a pharmaceutically acceptable excipient or carrier.

In one aspect, disclosed herein is a method of treating a disease or disorder characterized by overexpression of EphA2, comprising administering to the subject a radiopharmaceutical conjugate or a salt thereof as described herein.

In one aspect, disclosed herein is a kit for use in a method of diagnosing disease or disorder characterized by over/decreased expression of EphA2 by determination of the expression level of EphA2, wherein the kit comprising a radiopharmaceutical conjugate or a salt thereof as described herein.

In one aspect, disclosed herein is a composition for use in a method of diagnosing disease or disorder characterized by over/decreased expression of EphA2, wherein the composition comprising a radiopharmaceutical conjugate or a salt thereof as described herein.

In one aspect, disclosed herein is the use of a radiopharmaceutical conjugate or a salt thereof as described herein for use in a method of diagnosing disease or disorder characterized by over/decreased expression of EphA2.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing (also “figure” and “FIG.” herein), of which:

FIG. 1 illustrates the structures of exemplary conjugates of the present disclosure, including a peptide, a linker and a metal chelator. FIG. 1 discloses SEQ ID NOS 296, 433, 424, 434, 435, 436 and 437, respectively, in order of appearance.

FIG. 2 illustrates the structures of exemplary conjugates of the present disclosure, including a peptide, a linker, a metal chelator, a cold lutetium. FIG. 2 discloses SEQ ID NOS 292, 330, 283, 328, 334, 360 and 361, respectively, in order of appearance.

FIG. 3 illustrates the structures of exemplary conjugates of the present disclosure, including a peptide, a linker, and a metal chelator.

FIG. 4A illustrates exemplary metal chelators of the present disclosure, wherein [symbol] represents the attachment point of a metal chelator to the remaining conjugate. FIG. 4B illustrates the same metal chelators as FIG. 4A, except that a part of the linker or the peptide covalently connected to the metal chelator is shown in the dashed circle [symbol] .

FIG. 5A illustrates exemplary metal chelators of the present disclosure, wherein [symbol] represents the attachment point of a metal chelator to the remaining conjugate. FIG. 5B illustrates the same metal chelators as FIG. 5A, except that a part of the linker or the peptide covalently connected to the metal chelator is shown in the dashed circle [symbol] .

FIG. 6A illustrates exemplary metal chelators of the present disclosure, wherein [symbol] represents the attachment point of a metal chelator to the remaining conjugate. FIG. 6B illustrates the same metal chelators as FIG. 6A, except that a part of the linker or the peptide covalently connected to the metal chelator is shown in the dashed circle [symbol] .

FIG. 7A illustrates exemplary metal chelators of the present disclosure, wherein [symbol] represents the attachment point of a metal chelator to the remaining conjugate. FIG. 7B illustrates the same metal chelators as FIG. 7A, except that a part of the linker or the peptide covalently connected to the metal chelator is shown in the dashed circle [symbol] .

FIG. 8 illustrates the structures of representative metal chelators.

FIG. 9 illustrates the structures of representative metal chelators.

FIG. 10 illustrates the structures of representative metal chelators.

FIG. 11 illustrates the structures of representative metal chelators.

FIG. 12 illustrates the structures of representative metal chelators.

FIG. 13 illustrates the structures of representative metal chelators.

FIG. 14 illustrates the structures of representative metal chelators.

FIG. 15 illustrates the structures of representative metal chelators.

FIG. 16 illustrates the structures of representative metal chelators.

FIG. 17 illustrates the structures of representative metal chelators.

FIG. 18 illustrates the structures of representative metal chelators.

FIG. 19 illustrates the structures of representative metal chelators.

FIG. 20 illustrates the structures of representative metal chelators.

FIG. 21 illustrates the structures of representative metal chelators.

FIG. 22 illustrates the structures of representative metal chelators.

FIG. 23 illustrates cell binding of biotinylated compounds EphA2-Biotin-21 and EphA2-Biotin-88 tested in HCT116 cells and the binding EC50.

FIG. 24A illustrates the competition cell binding for PDC_EphA2-00007196-C302, PDC_EphA2-00019440-C302, and PDC_EphA2-00019443-C302 tested against 50 nM of EphA2-Biotin-88 in HCT116 cells; FIG. 24B illustrates the competition cell binding for PDC_EphA2-00001417-C304 with the biotinylated form of a reference bicyclic peptide in H1299 cells.

FIG. 25 illustrates the internalization rate of biotinylated compound EphA2-Biotin-21 and EphA2-Biotin-88 measured in PC3 cells at 10 nM and 100 nM at 2 hour time point.

FIG. 26 illustrates the results of the SPR peptide binding study for PDC_EphA2-00007196-C302, PDC_EphA2-00019443-C302, PDC_EphA2-00019440-C302, and PDC_EphA2-00008010-C302. X axis represents time(s) and Y axis is response unit (RU).

FIG. 27 illustrates the structures of exemplary conjugates comprising covalently bound radionuclides of the present disclosure. FIG. 27 discloses SEQ ID NOS 88, 171, 114, 55, 438-440, respectively, in order of appearance.

FIG. 28 illustrates the structures of exemplary conjugates comprising covalently bound radionuclides of the present disclosure. FIG. 28 discloses 441-443, respectively, in order of appearance.

FIG. 29 illustrates the structures of exemplary conjugates comprising covalently bound radionuclides of the present disclosure, including a peptide, a linker, and a radionuclide.

### DETAILED DESCRIPTION

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### I. DEFINITIONS

As used in the specification and appended claims, unless specified to the contrary, the following terms have the meaning indicated below.

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value.

The term “comprising” (and related terms such as “comprise” or “comprises” or “having” or “including”) are to be construed in an open, inclusive sense, that is, as “including, but not limited to.” The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Amino” refers to the —NH₂ radical.

“Cyano” refers to the —CN radical.

“Nitro” refers to the —NO₂ radical.

“Oxo” refers to the —O radical.

“Imino” refers to the ═N—H radical.

“Oximo” refers to the ═N—OH radical.

“Hydrazino” refers to the ═N—NH₂ radical.

“Hydroxy” or “hydroxyl” refers to the —OH radical.

“Hydroxyamino” refers to the —NH—OH radical.

“Acyl” refers to a substituted or unsubstituted alkylcarbonyl, substituted or unsubstituted alkenylcarbonyl, substituted or unsubstituted alkynylcarbonyl, substituted or unsubstituted cycloalkylcarbonyl, substituted or unsubstituted heterocycloalkylcarbonyl, substituted or unsubstituted arylcarbonyl, substituted or unsubstituted heteroarylcarbonyl, amide, or ester, wherein the carbonyl atom of the carbonyl group is the point of attachment. Unless stated otherwise specifically in the specification, an alkylcarbonyl group, alkenylcarbonyl group, alkynylcarbonyl group, cycloalkylcarbonyl group, amide group, or ester group is optionally substituted, for example, with oxo, halogen, amino, nitrile, nitro, hydroxyl, haloalkyl, alkoxy, aryl, cycloalkyl, heterocycloalkyl, heteroaryl, and the like.

“Alkyl” refers to an optionally substituted straight-chain, or optionally substituted branched-chain saturated hydrocarbon monoradical. An alkyl group can have from one to about twenty carbon atoms, from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, —NO₂, or —C≡CH. In some embodiments, the alkyl is optionally substituted with oxo, halogen, —CN, —CF₃, —OH, or —OMe. In some embodiments, the alkyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen. In some embodiments, the alkylene is —CH₂—, —CH₂CH₂—, —CH₂CH₂CH₂—, or —CH₂CH(CH₃)CH₂—. In some embodiments, the alkylene is —CH₂—. In some embodiments, the alkylene is —CH₂CH₂—. In some embodiments, the alkylene is —CH₂CH₂CH₂—.

“Alkenyl” refers to an optionally substituted straight-chain, or optionally substituted branched-chain hydrocarbon monoradical having one or more carbon-carbon double-bonds. In some embodiments, an alkenyl group has from two to about ten carbon atoms, or two to about six carbon atoms. The group may be in either the cis or trans configuration about the double bond(s), and should be understood to include both isomers. Examples include, but are not limited to, ethenyl (—CH═CH₂), 1-propenyl (—CH₂CH═CH₂), isopropenyl [—C(CH₃)—CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

The term “alkenylene” or “alkenylene chain” refers to an optionally substituted straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

“Alkynyl” refers to an optionally substituted straight-chain or optionally substituted branched-chain hydrocarbon monoradical having one or more carbon-carbon triple-bonds. In some embodiments, an alkynyl group has from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen. The term “alkynylene” refers to an optionally substituted straight-chain or optionally substituted branched-chain divalent hydrocarbon having one or more carbon-carbon triple-bonds.

“Alkylamino” refers to a radical of the formula —N(Rₐ)₂ where Rₐ is an alkyl radical as defined, or two Rₐ, taken together with the nitrogen atom, can form a substituted or unsubstituted C₂-C₇ heterocyloalkyl ring. Unless stated otherwise specifically in the specification, an alkylamino group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylamino is optionally substituted with oxo, halogen, —CN, —CF₃, —OH, —OMe, —NH₂, or —NO₂. In some embodiments, an alkylamino is optionally substituted with oxo, halogen, —CN, —CF₃, —OH, or —OMe. In some embodiments, the alkylamino is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems. Unless stated otherwise specifically in the specification, an aryl may be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1 (2H)-one, spiro[2.2]pentyl, norbornyl and bicycle[1.1.1]pentyl. Unless otherwise stated specifically in the specification, a cycloalkyl group may be optionally substituted. Representative cycloalkyls include, but are not limited to, cycloalkyls having from three to fifteen carbon atoms (C₃-C₁₅ cycloalkyl), from three to ten carbon atoms (C3-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C3-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro, or iodo. In some embodiments, halogen is fluoro or chloro. In some embodiments, halogen is fluoro.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl can include, for example, iodoalkyl, bromoalkyl, chloroalkyl, and fluoroalkyl. For example, “fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen. As used herein, a “heteroalkylene” refers to divalent heteroalkyl group. Examples of such heteroalkylene are, for example, —CH2-O—CH2-, —CH2-N(alkyl)-CH2-, —CH2-N(aryl)-CH2-, —OCH2CH2O—, —OCH2CH2OCH2CH2O—, or —OCH2CH2OCH2CH2OCH2CH2O—. Unless stated otherwise, a heteroalkylene can be optionally substituted.

The term “heterocycloalkyl” refers to a cycloalkyl group that includes at least one hetero ring atom, e.g., a heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a ring system radical comprising carbon atom(s) and one or more ring heteroatoms selected from the group consisting of nitrogen, oxygen, phosphorous, and sulfur, and at least one aromatic ring. In some embodiments, heteroaryl is monocyclic, bicyclic or polycyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C9 heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems. Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The term “moiety” refers to a specific segment or functional group of a molecule. Chemical moieties are often recognized chemical entities embedded in or appended to a molecule.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. As used herein, “treating” includes the concepts of “alleviating”, which refers to lessening the frequency of occurrence or recurrence, or the severity, of any symptoms or other ill effects related to a disorder and/or the associated side effects. The term “treating” also encompasses the concept of “managing” which refers to reducing the severity of a particular disease or disorder in a patient or delaying its recurrence, e.g., lengthening the period of remission in a patient who had suffered from the disease.

In certain embodiments, the term “prevent” or “preventing” as related to a disease or disorder can refer to a compound that in a statistical sample, reduces the occurrences of the disorder or condition in the treated sample relative to an untreated control sample, or delays the onset or reduces the severity of one or more symptoms of the disorder or condition relative to the untreated control sample.

The term “therapeutically effective amount” as used herein to refer to an amount effective at the dosage and duration necessary to achieve the desired therapeutic result. A therapeutically effective amount of the composition may vary depending on factors such as the individual's condition, age, sex, and weight, and the ability of the protein to elicit the desired response of the individual. A therapeutically effective amount can also be an amount that exceeds any toxic or deleterious effect of the composition that would have a beneficial effect on the treatment.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be unsubstituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure. A person of ordinary skill in the art would understand that, while only a single bond is shown, a double bond is intended for those substituents.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, oxo, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, oxo, —CO₂H, —CO₂ (C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —NH (cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O). When indicating the number of substituents, the term “one or more” means from one substituent to the highest possible number of substitutions, i.e. replacement of one hydrogen up to replacement of all hydrogens by substituents.

The term “unsubstituted” means that the specified group bears no substituents.

Certain compounds described herein may exist in tautomeric forms, and all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

The term “peptide” as used herein refers to a compound that includes two or more amino acids. A peptide described herein can comprise one or more unnatural amino acids. The term “peptide” also encompasses peptide mimetics. In the present disclosure, the term “amino acid” is used in its broadest meaning and it embraces not only natural amino acids but also derivatives thereof and artificial amino acids. For example, the term “amino acid” encompasses unnatural amino acids.

As used herein, the term “unnatural amino acid” refers to an amino acid other than the 20 canonical amino acids. The 20 canonical amino acids refer to alanine (ala or A), arginine (arg or R), asparagine (asn or N), aspartic acid (asp or D), cysteine (cys or C), glutamine (gln or Q), glutamic acid (glu or E), glycine (gly or G), histidine (his or H), isoleucine (ile or I), leucine (leu or L), lysine (lys or K), methionine (met or M), phenylalanine (phe or F), proline (pro or P), serine (ser or S), threonine (thr or T), tryptophan (trp or W), tyrosine (tyr or Y), and valine (val or V).

The term “protein” as used herein refers to a polypeptide (i.e., a string of at least 3 amino acids linked to one another by peptide bonds). Proteins can include moieties other than amino acids (e.g., may be glycoproteins, proteoglycans, etc.) and/or can be otherwise processed or modified. A protein can be a complete polypeptide as produced by and/or active in a cell (with or without a signal sequence). In some embodiments, a protein is or comprises a characteristic portion such as a polypeptide as produced by and/or active in a cell. A protein can include more than one polypeptide chain. For example, polypeptide chains can be linked by one or more disulfide bonds or associated by other means.

The term “peptide mimetic” or “mimetic” refers to biologically active compounds that mimic the biological activity of a peptide or a protein but are no longer entirely peptidic in chemical nature, e.g., they can contain non-peptide bonds (that are, bonds other than amide bonds between amino acids). As used herein, the term peptide mimetic is used in a broader sense to include molecules that are no longer completely peptidic in nature, such as pseudo-peptides, semi-peptides and peptoids. Whether completely or partially non-peptide, peptide mimetics described herein can provide a spatial arrangement of reactive chemical moieties that closely resemble the three-dimensional arrangement of active groups in the subject amino acid sequence or subject molecule on which the peptide mimetic is based. As a result of this similar active-site geometry, the peptide mimetic can have effects on biological systems that are similar to the biological activity of the subject entity.

In some embodiments, the peptide mimetics are substantially similar in both three-dimensional shape and biological activity to the subject amino acid sequence or subject molecule on which the peptide mimetic is based. An example is described in the paper “Tritiated D-alal-Peptide T Binding”, Smith C. S. et al., Drug Development Res., 15, pp. 371-379 (1988). A second method is altering cyclic structure for stability, such as N to C interchain imides and lactams (Ede et al. in Smith and Rivier (Eds.) “Peptides: Chemistry and Biology”, Escom, Leiden (1991), pp. 268-270). An example of this is provided in conformationally restricted thymopentin-like compounds, such as those disclosed in U.S. Pat. No. 4,457,489. A third method is to substitute peptide bonds in the subject entity by pseudopeptide bonds that confer resistance to proteolysis.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, C₁-Cₓ (or C₁₋ₓ) includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂—, and —CH₂CH₂-linkages.

The term “cyclized” or “cyclization” as used herein means that two amino acids apart from each other by at least one amino acid bind directly or bind indirectly to each other in one peptide to form a cyclic structure in the molecule. In some cases, the two amino acids bind via a linker or the like.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a companion animal such as a dog or a cat. In one aspect, the mammal is a human.

The term “therapeutically effective amount” as used herein to refer to an amount effective at the dosage to achieve the desired therapeutic result. A therapeutically effective amount of a composition may vary depending on factors such as the individual's condition (e.g., age, sex, and weight), the radiopharmaceutical conjugate, and the method of administration (e.g., oral or parenteral).

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The Smith Waterman algorithm can also be used to determine percent identity. Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Nat. Acad. Sci. USA., 89:10915-10919 (1992)) 3) gap penalty=12; and 4) gap length penalty=4. A program useful with these parameters can be publicly available as the “gap” program (Genetics Computer Group, Madison, Wis.). The aforementioned parameters are the default parameters for polypeptide comparisons (with no penalty for end gaps). Alternatively, polypeptide sequence identity can be calculated using the following equation: % identity−(the number of identical residues)/(alignment length in amino acid residues)*100. For this calculation, alignment length includes internal gaps but does not include terminal gaps.

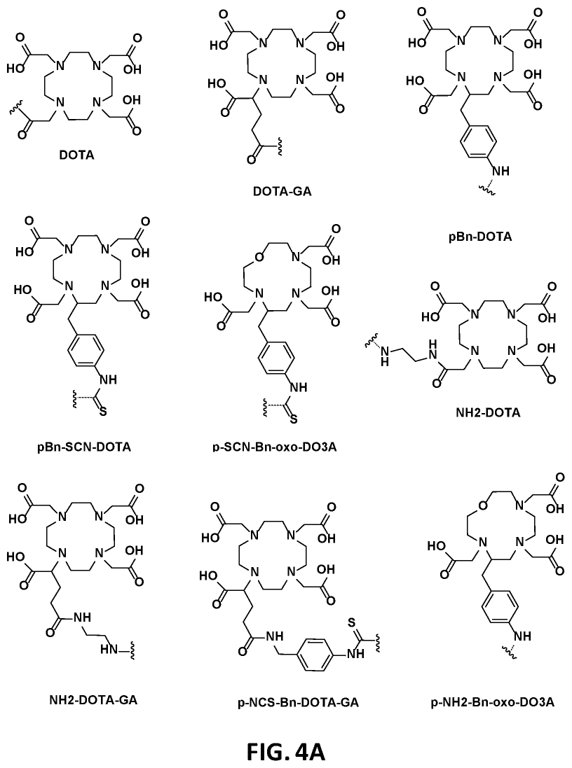

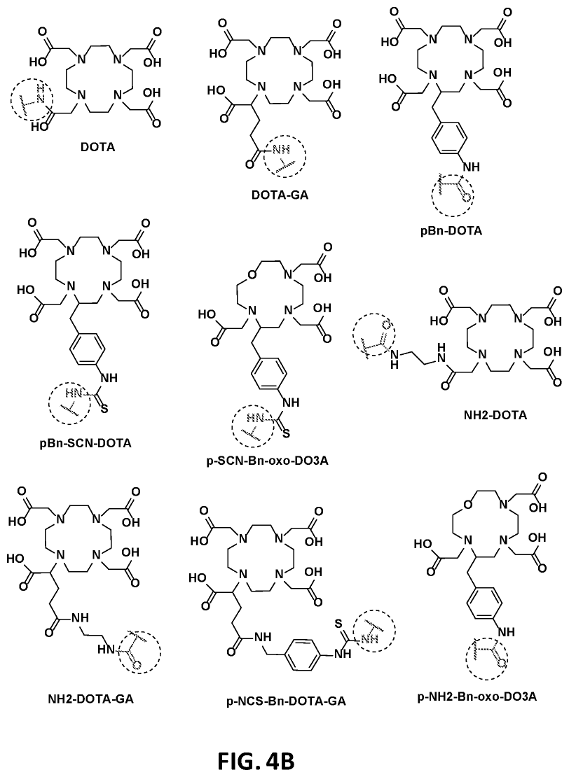

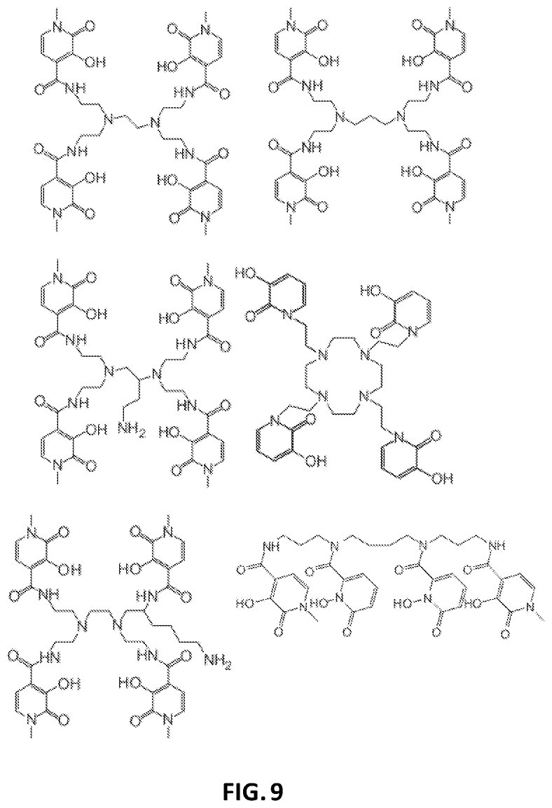

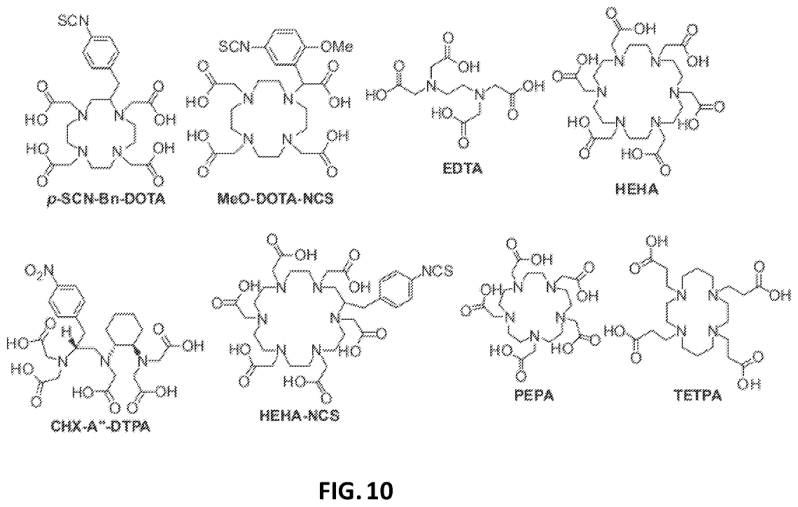

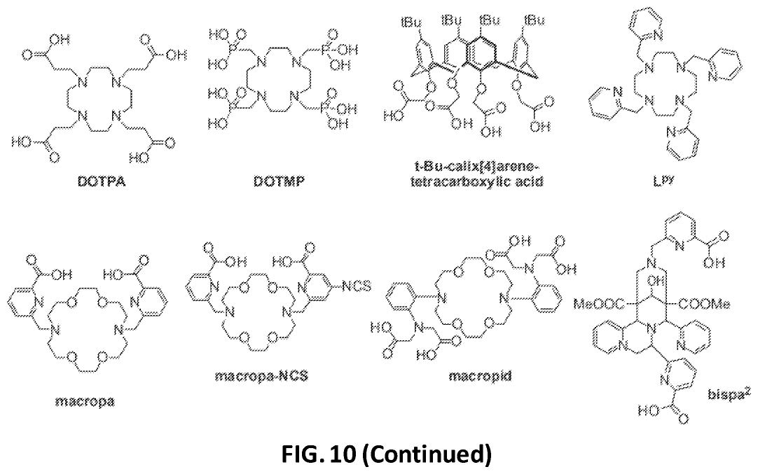

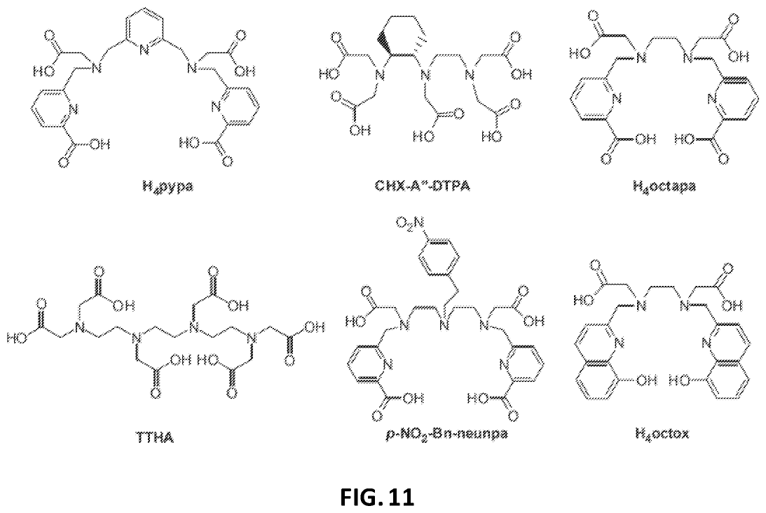

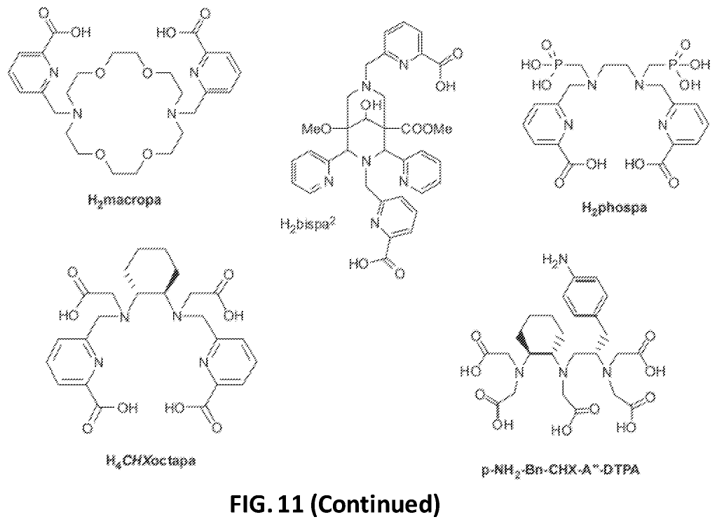

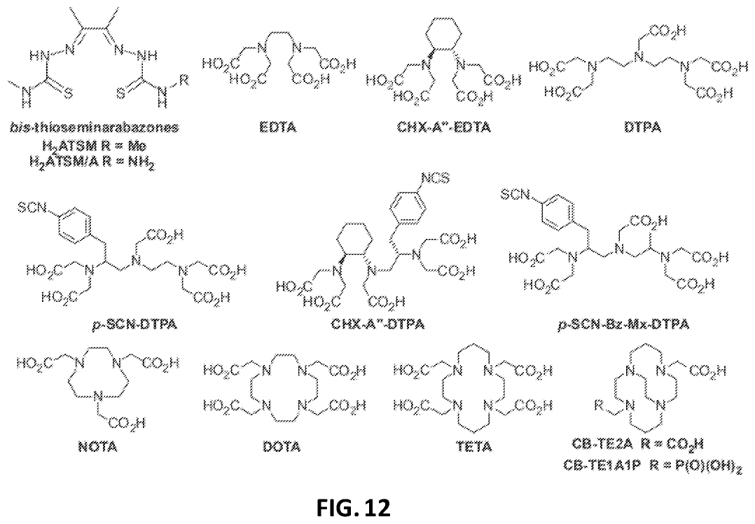

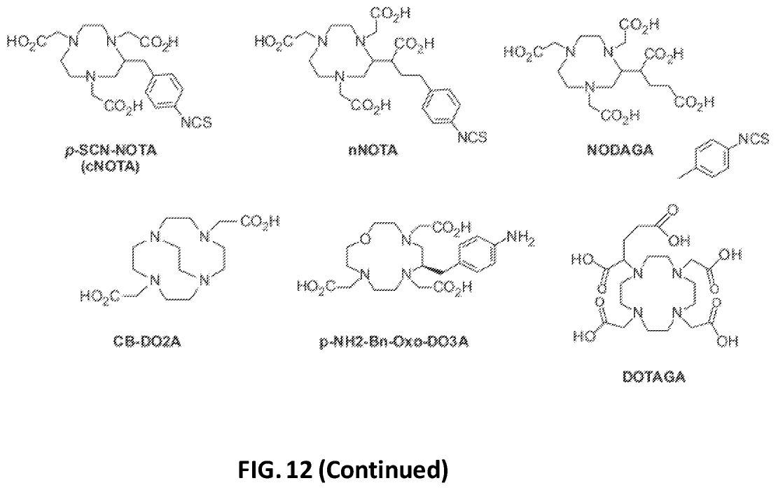

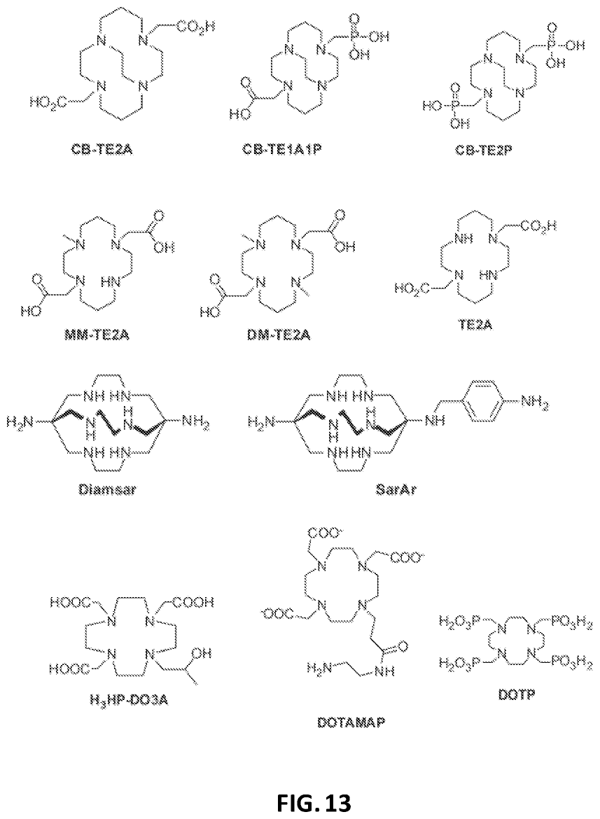

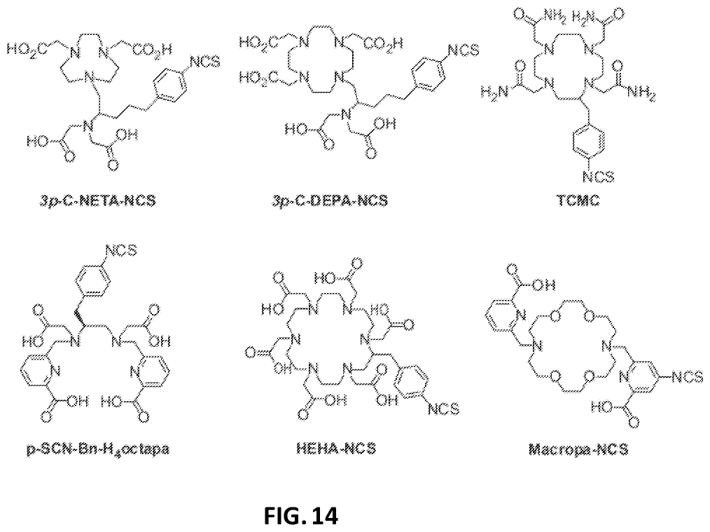

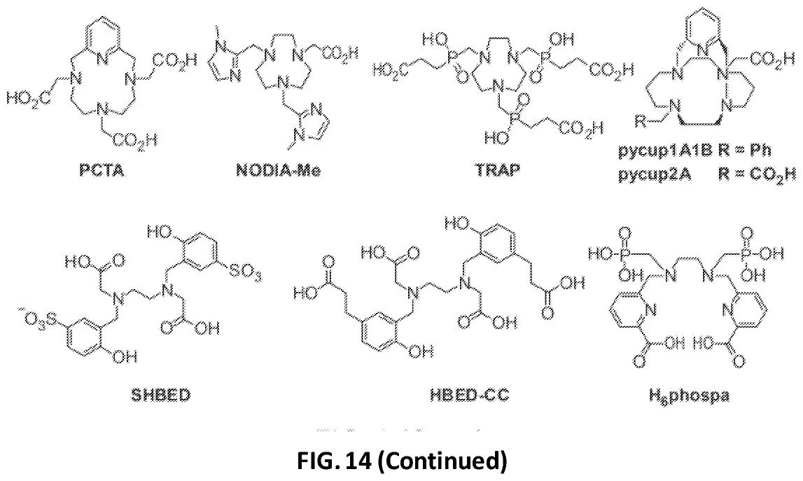

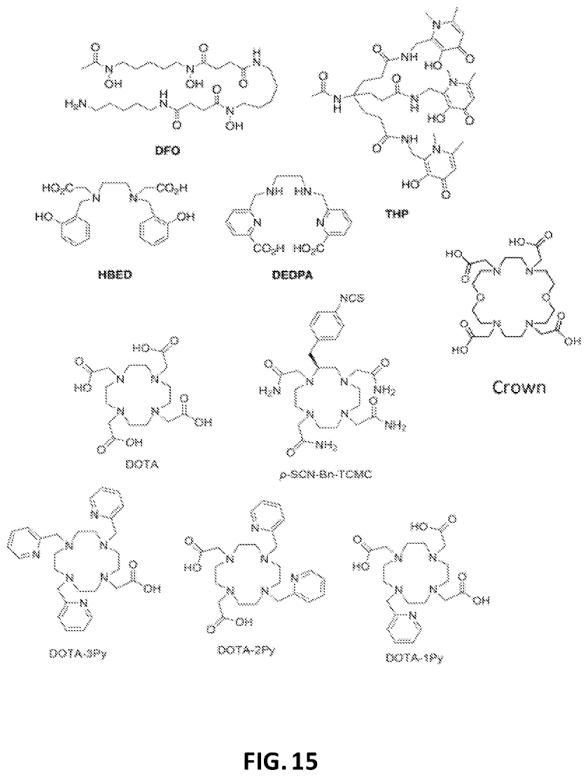

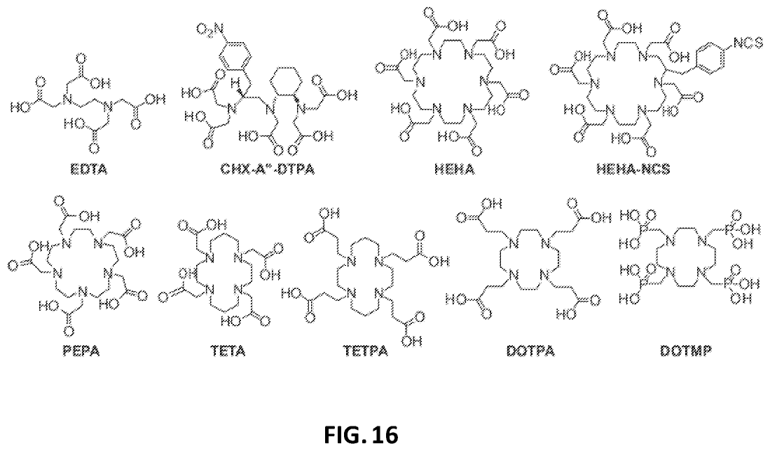

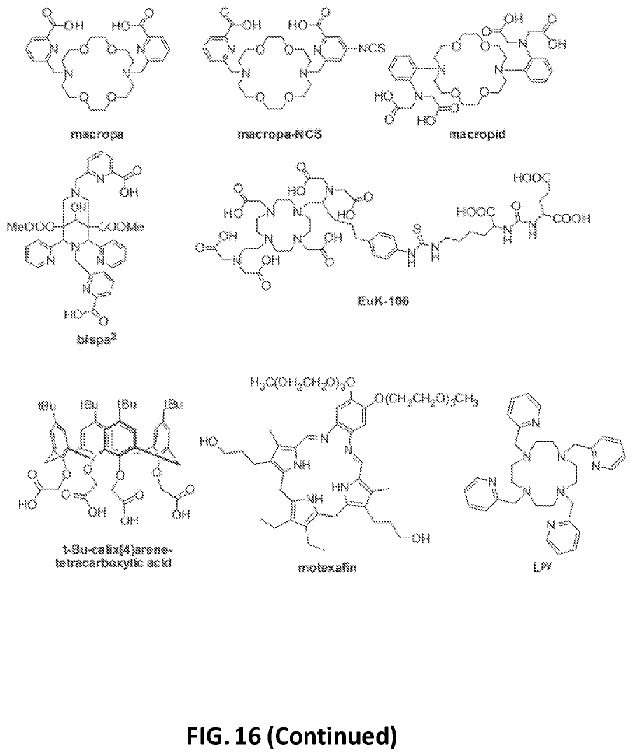

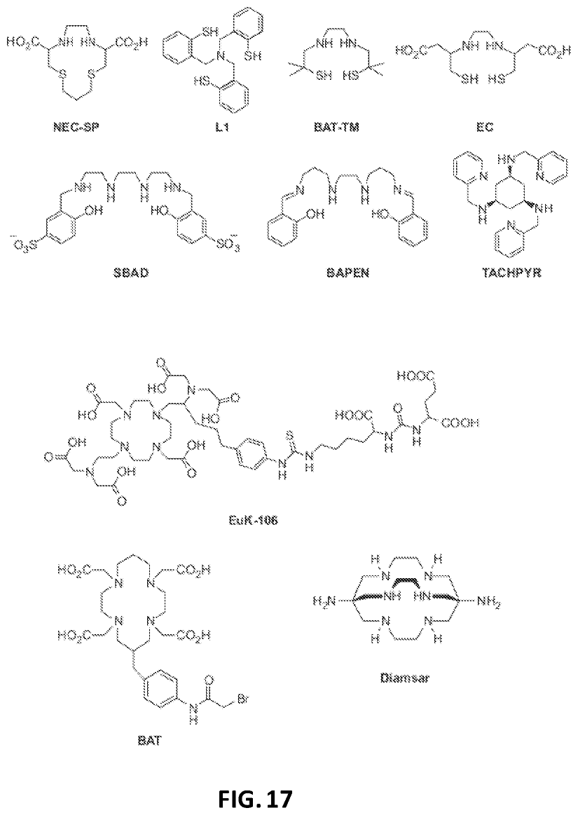

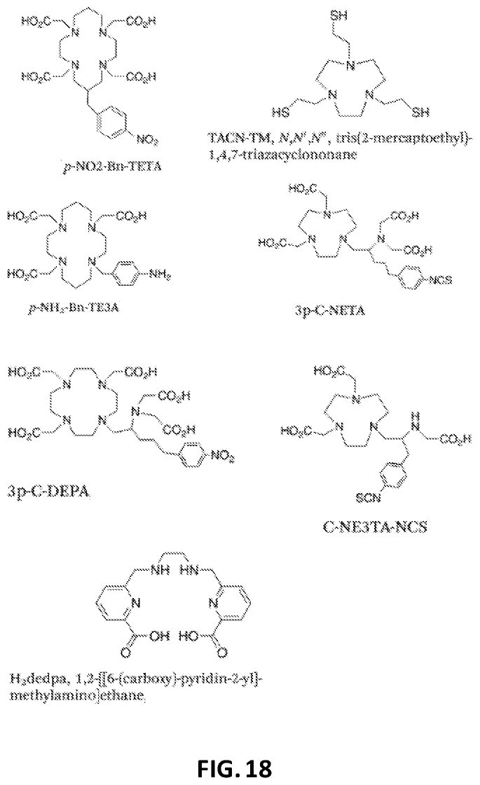

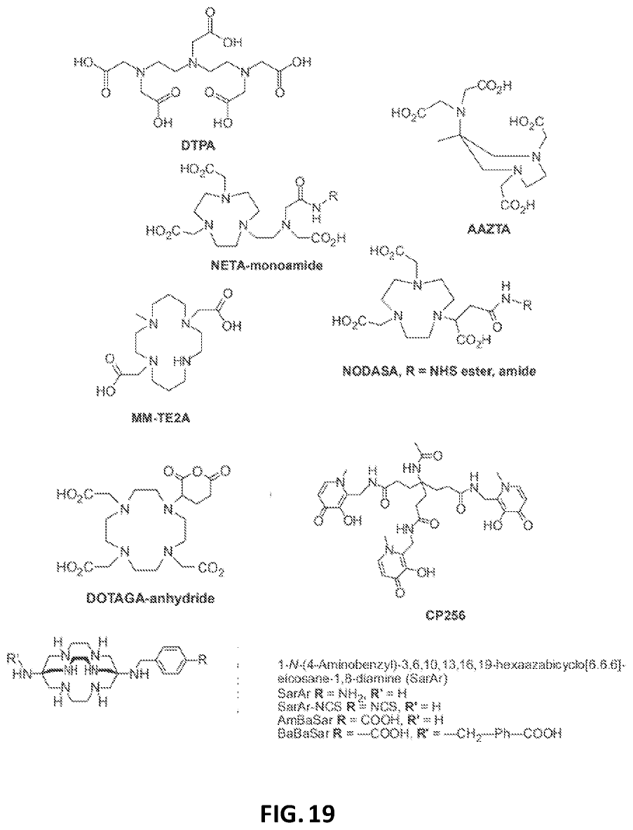

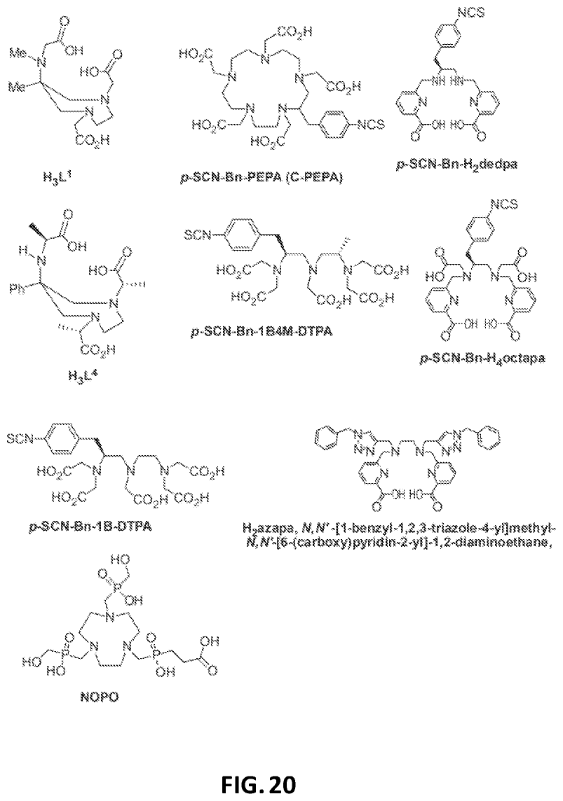

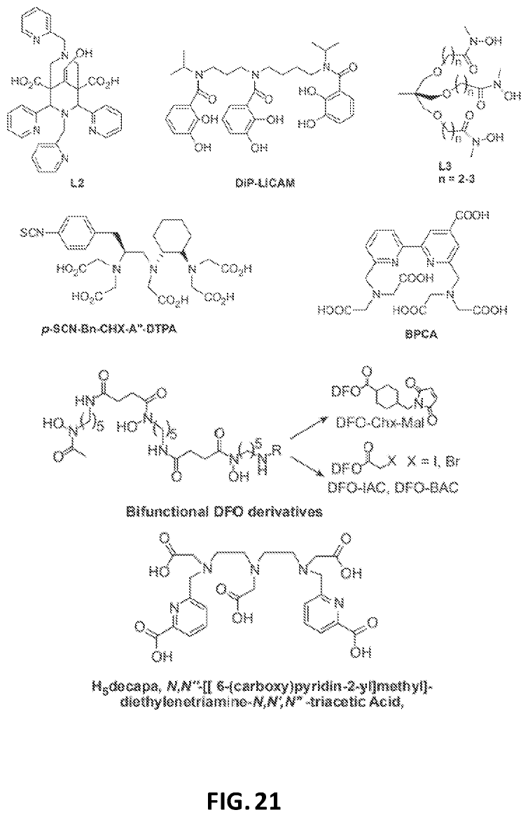

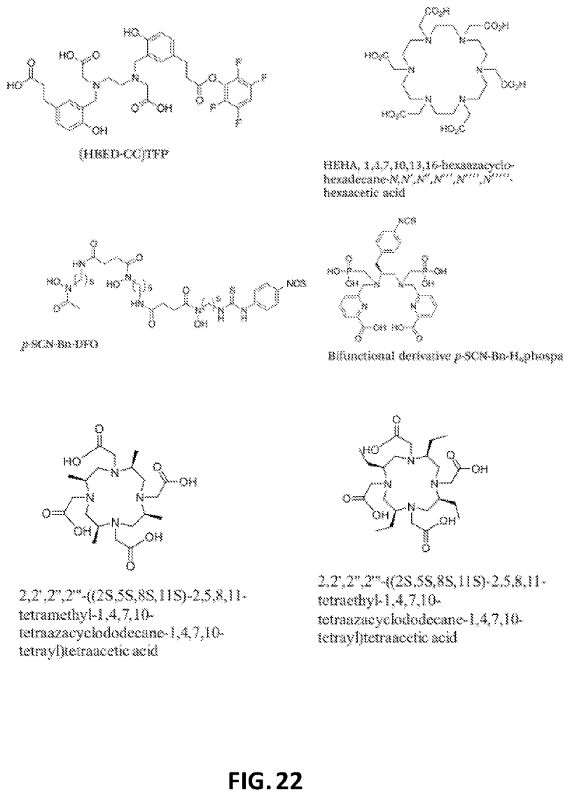

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable subcombination. For example, a conjugate of this disclosure can comprise any peptide ligand described herein (e.g., a peptide ligand of Formula (I), (I-1), (I-2), (I-3), (I-4), (I-5), (Ia), (Ib), or (Ic), or Table 1), any metal chelator described herein (e.g., a metal chelator selected from FIGS. 4A, 5A, 6A, 7A, 4B, 5B, 6B, 7B and 8-22), optionally a linker described herein (e.g., a linker of Formula (II-1), (II-1a), (II-1b), or (II-2)), and optionally a radionuclide described herein (e.g., a radionuclide of Table 7 labeled “chelator”). As another example, a conjugate of this disclosure can comprise any peptide ligand described herein (e.g., a peptide ligand of Formula (I), (I-1), (I-2), (I-3), (I-4), (I-5), (Ia), (Ib), or (Ic), or Table 1), any covalent radionuclide described herein (e.g., a radionuclide of Table 7 labeled “covalent”), and optionally a linker described herein (e.g., a linker of Formula (II-1), (II-1a), (II-1b), or (II-2) or Table 6) connecting the covalent radionuclide to the peptide. For another example, a peptide of Formula (I) (or any other formulas such as (III-1), (III-2), (III-1-RI), and (III-2-RI)) can comprise X1 to X12 amino acids as described herein, and any combinations of the embodiments of amino acids are encompassed by this disclosure (even though, in some cases, they are described in the context of separate embodiments).

### II. RADIOPHARMACEUTICAL CONJUGATES

Provided herein are radiopharmaceutical conjugates that have avidity for ephrin type-A receptor 2 (EphA2) and pharmaceutical compositions comprising the conjugates. The conjugates and compositions can be useful for treating cancer. The conjugates and compositions can also be useful in imaging and disease diagnosis.

In one aspect, described herein is a conjugate that comprises a peptide that has avidity for ephrin type-A receptor 2 (EphA2) and a metal chelator that is configured to bind with a radionuclide. In some embodiments, the EphA2 is a human EphA2. In some embodiments, the conjugate or the peptide described herein does not have avidity toward human EphA1, EphA3, EphA4, EphA5, EphA6, EphA7 or EphB4. In some embodiments, the conjugate or the peptide described herein does not exhibit significant binding to human EphA1, EphA3, EphA4, EphA5, EphA6, EphA7 or EphB4. The peptide can be cyclic or acyclic, and it can be monocyclic, bicyclic or polycyclic. In one aspect, described herein is a conjugate that comprises a cyclic peptide and a metal chelator that is configured to bind with a radionuclide. In some embodiments, the peptide (such as cyclic peptide) is configured to bind to a target. A conjugate described herein can further comprises a linker that covalently attaches the peptide to the metal chelator. In some embodiments, the conjugate comprises a radionuclide such as ²²⁵Ac bound to the metal chelator.

In another aspect, described herein is a conjugate that comprises a peptide that has avidity for ephrin type-A receptor 2 (EphA2) and a covalently bound radionuclide. In some embodiments, the EphA2 is a human EphA2. In some embodiments, the conjugate or the peptide described herein does not have avidity toward human EphA1, EphA3, EphA4, EphA5, EphA6, EphA7 or EphB4. In some embodiments, the conjugate or the peptide described herein does not exhibit significant binding to human EphA1, EphA3, EphA4, EphA5, EphA6, EphA7 or EphB4. The peptide can be cyclic or acyclic, and it can be monocyclic, bicyclic or polycyclic. In one aspect, described herein is a conjugate that comprises a cyclic peptide and a covalently bound radionuclide. In some embodiments, the peptide (such as cyclic peptide) is configured to bind to a target. A conjugate described herein can further comprises a linker that covalently attaches the peptide to the radionuclide. In some embodiments, the conjugate comprises a covalently bound radionuclide such as ¹³¹I.

In one aspect, described herein is a radiopharmaceutical conjugate comprising:
(a) a peptide that has avidity for ephrin type-A receptor 2 (EphA2), wherein the peptide comprises an amino acid sequence including deletion, substitution, and/or addition of one or several amino acids in the amino acid of SEQ ID NO:1:

(SEQ ID NO: 1)
da-MeF-N-L-Hgl-MeF-W1Me-V-W1Me-T-E-C

or a pharmaceutically acceptable salt thereof; and
(b)(i) a metal chelator configured to bind with a radionuclide, wherein the metal chelator is conjugated to the peptide; or (ii) a covalent radionuclide (or, a radionuclide covalently bound to the peptide). In some embodiments, the peptide consists of 7, 8, 9, 10, 11, 12, or 13 amino acid residues. In some embodiments, the radiopharmaceutical conjugate comprises a metal chelator configured to bind with a radionuclide, wherein the metal chelator is conjugated to the peptide. In some embodiments, the metal chelator is covalently connected to the peptide. In some embodiments, the radiopharmaceutical conjugate comprises a covalent radionuclide. In some embodiments, the radiopharmaceutical conjugate comprises a radionuclide covalently bound to the peptide.

In some embodiments, the peptide is a cyclic peptide. In some embodiments, the peptide consists of 10 or 12 amino acid residues. In some embodiments, the peptide comprises an amino acid sequence with deletion of 2 or less amino acids in the amino acid SEQ ID NO: 1. In some embodiments, 1-2 amino acids selected from the group consisting of 10th T and 11th E of SEQ ID NO: 1 is deleted. In some embodiments, the 8^{th} V is of SEQ ID NO: 1 is substituted. In some embodiments, the 11^{th} E of SEQ ID NO: 1 is substituted.

In one aspect, described herein is a radiopharmaceutical conjugate comprising:
(a) a peptide that has avidity for ephrin type-A receptor 2 (EphA2), wherein the peptide has an amino acid sequence according to Formula (I), or a pharmaceutically acceptable salt thereof,

X1-X2-X3-X4-X5-X6-X7-X8-X9-X10-X11-X12 Formula (I)
wherein,
   X1 is an amino acid;
   X2 is an amino acid comprising an aromatic ring, an N-methylated amino acid thereof, or a variant thereof;
   X3 is a hydrophilic amino acid (e.g. N, Q, Cit, K or a variant thereof), glycine (G), Alanine (A) or a variant thereof (e.g., da, 2-Aminoisobutyric acid (Aib));
   X4 is a hydrophobic amino acid (e.g., leucine (L)), a hydrophilic amino acid (e.g., citrulline (Cit)), or a variant thereof;
   X5 is a hydrophilic amino acid, or a variant thereof;
   X6 is a hydrophilic amino acid, an amino acid comprising an aromatic ring, or an N-methylated amino acid thereof;
   X7 is an amino acid comprising an aromatic ring (e.g., W, F, or a variant thereof);
   X8 is a hydrophobic amino acid, a hydrophilic amino acid, an N-methylated amino acid, or a variant thereof;
   X9 is an amino acid comprising an aromatic ring (e.g., W or a variant thereof);
   X10 is absent or a hydrophilic amino acid (e.g., Threonine (T) or a variant thereof);
   X11 is absent or a hydrophilic amino acid; and
   X12 is cysteine (C) or a variant thereof; and
(b)(i) a metal chelator configured to bind with a radionuclide, wherein the metal chelator is conjugated to the peptide; or (ii) a covalent radionuclide.

In some embodiments, the peptide is a cyclic peptide. In some embodiments, the radiopharmaceutical conjugate comprises a metal chelator configured to bind with a radionuclide, wherein the metal chelator is conjugated to the peptide. In some embodiments, the metal chelator is covalently connected to the peptide. In some embodiments, the radiopharmaceutical conjugate comprises a covalent radionuclide. In some embodiments, the radiopharmaceutical conjugate comprises a radionuclide covalently bound to the peptide. In some embodiments, the peptide is a cyclic peptide. In some embodiments, the peptide is a monocyclic peptide.

In one aspect, described herein is a radiopharmaceutical conjugate comprising:
(a) a peptide that has avidity for ephrin type-A receptor 2 (EphA2), wherein the peptide has an amino acid sequence according to Formula (I), or a pharmaceutically acceptable salt thereof,

X1-X2-X3-X4-X5-X6-X7-X8-X9-X10-X11-X12 Formula (I)
wherein,
   X1 is an amino acid;
   X2 is an amino acid comprising an aromatic ring, an N-methylated amino acid thereof, or a variant thereof;
   X3 is absent, a hydrophilic amino acid (e.g. N, Q, Cit, K or a variant thereof), glycine (G), Alanine (A) or a variant thereof (e.g., da, 2-Aminoisobutyric acid (Aib));
   X4 is absent, a hydrophobic amino acid (e.g., leucine (L)), a hydrophilic amino acid (e.g., citrulline (Cit)), or a variant thereof;
   X5 is absent, a hydrophilic amino acid, or a variant thereof;
   X6 is absent, a hydrophilic amino acid, an amino acid comprising an aromatic ring, or an N-methylated amino acid thereof;
   X7 is an amino acid comprising an aromatic ring (e.g., W, F, or a variant thereof);
   X8 is a hydrophobic amino acid, a hydrophilic amino acid, an N-methylated amino acid, or a variant thereof;
   X9 is an amino acid comprising an aromatic ring (e.g., W or a variant thereof);
   X10 is absent or a hydrophilic amino acid (e.g., Threonine (T) or a variant thereof);
   X11 is absent or a hydrophilic amino acid; and
   X12 is cysteine (C) or a variant thereof; and
(b)(i) a metal chelator configured to bind with a radionuclide, wherein the metal chelator is conjugated to the peptide; or (ii) a covalent radionuclide.

In some embodiments, the peptide is a cyclic peptide. In some embodiments, the radiopharmaceutical conjugate comprises a metal chelator configured to bind with a radionuclide, wherein the metal chelator is conjugated to the peptide. In some embodiments, the metal chelator is covalently connected to the peptide. In some embodiments, the radiopharmaceutical conjugate comprises a covalent radionuclide. In some embodiments, the radiopharmaceutical conjugate comprises a radionuclide covalently bound to the peptide.

In one aspect, described herein is a radiopharmaceutical conjugate comprising:
(a) a peptide that has avidity for ephrin type-A receptor 2 (EphA2), wherein the peptide has an amino acid sequence according to Formula (I), or a pharmaceutically acceptable salt thereof,

X1-X2-X3-X4-X5-X6-X7-X8-X9-X10-X11-X12 Formula (I)
or a pharmaceutically acceptable salt thereof,
wherein
   each of X1, X2, X3, X4, X5, X6, and X8 is independently an amino acid;
   X7 is W1Me or a variant thereof;
   X9 is W1Me or a variant thereof;
   each of X10 and X11 is independently absent or an amino acid; and
   X12 is cysteine (C) or a variant thereof;
(b)(i) a metal chelator configured to bind with a radionuclide; or (ii) a covalent radionuclide; and
(c)(i) a linker that connects the peptide with the metal chelator; or (ii) a linker that connects the peptide with the covalent radionuclide. In some embodiments, the radiopharmaceutical conjugate comprises a metal chelator configured to bind with a radionuclide and a linker that connects the peptide with the metal chelator. In some embodiments, the metal chelator is covalently connected to the peptide. In some embodiments, the radiopharmaceutical conjugate comprises a covalent radionuclide and a linker that connects the peptide with the covalent radionuclide.

In some embodiments, the metal chelator is conjugated to the N-terminus of the peptide. In some embodiments, the conjugate further comprises a linker that connects the peptide with the metal chelator. In some embodiments, the linker covalently connects the peptide with the metal chelator. In some embodiments, the linker covalently attaches the metal chelator to the N-terminus of the peptide. In some embodiments, the linker covalently attaches the metal chelator to the C-terminus of the peptide. In some embodiments, the linker is attached to the peptide via a non-terminal amino acid residue of the peptide. In some embodiments, the linker is attached to amino acid X1. In some embodiments, the linker is attached to amino acid X2. In some embodiments, the linker is attached to amino acid X3. In some embodiments, the linker is attached to amino acid X4. In some embodiments, the linker is attached to amino acid X5. In some embodiments, the linker is attached to amino acid X6. In some embodiments, the linker is attached to amino acid X7. In some embodiments, the linker is attached to amino acid X8. In some embodiments, the linker is attached to amino acid X9. In some embodiments, the linker is attached to amino acid X10. In some embodiments, the linker is attached to amino acid X11. In some embodiments, the linker is attached to amino acid X12. In some embodiments, the linker is attached to amino acid X5, X8 or X11. In some embodiments, the linker is attached to a lysine of the peptide. In some embodiments, the linker comprises one or more amino acid residues. In some embodiments, the linker comprises a lysine residue, an alanine residue, or both.

In one aspect, described herein is a radiopharmaceutical conjugate with structure of

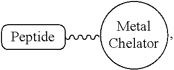

wherein [symbol] represents a linker.

In one aspect, described herein is a radiopharmaceutical conjugate with structure of

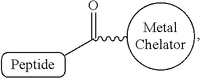

wherein
[symbol] represents the linker connected to the C-terminus of the peptide.

In some embodiments, the radiopharmaceutical conjugate comprises a covalent radionuclide. In some embodiments, the covalent radionuclide is attached to the N-terminus of the peptide. In some embodiments, the conjugate further comprises a linker that connects the peptide with the covalent radionuclide. In some embodiments, the linker covalently connects the peptide with the covalent radionuclide. In some embodiments, the linker covalently attaches the covalent radionuclide to the N-terminus of the peptide. In some embodiments, the linker covalently attaches the covalent radionuclide to the C-terminus of the peptide. In some embodiments, the linker is attached to the peptide via a non-terminal amino acid residue of the peptide. In some embodiments, the linker is attached to amino acid X1. In some embodiments, the linker is attached to amino acid X2. In some embodiments, the linker is attached to amino acid X3. In some embodiments, the linker is attached to amino acid X4. In some embodiments, the linker is attached to amino acid X5. In some embodiments, the linker is attached to amino acid X6. In some embodiments, the linker is attached to amino acid X7. In some embodiments, the linker is attached to amino acid X8. In some embodiments, the linker is attached to amino acid X9. In some embodiments, the linker is attached to amino acid X10. In some embodiments, the linker is attached to amino acid X11. In some embodiments, the linker is attached to amino acid X12. In some embodiments, the linker is attached to amino acid X5, X8 or X11. In some embodiments, the linker is attached to a lysine of the peptide. In some embodiments, the linker comprises one or more amino acid residues. In some embodiments, the linker comprises a lysine residue, an alanine residue, or both.

In some embodiments, the covalent radionuclide is bound directly to the peptide. In some embodiments, the covalent radionuclide is bound directly to the peptide via a non-terminal amino acid residue of the peptide. In some embodiments, the covalent radionuclide is bound to an aromatic amino acid in the peptide. In some embodiments, the covalent radionuclide is bound to amino acid X1. In some embodiments, the covalent radionuclide is bound to amino acid X2. In some embodiments, the covalent radionuclide is bound to amino acid X3. In some embodiments, the covalent radionuclide is bound to amino acid X4. In some embodiments, the covalent radionuclide is bound to amino acid X5. In some embodiments, the covalent radionuclide is bound to amino acid X6. In some embodiments, the covalent radionuclide is bound to amino acid X7. In some embodiments, the covalent radionuclide is bound to amino acid X8. In some embodiments, the covalent radionuclide is bound to amino acid X9. In some embodiments, the covalent radionuclide is bound to amino acid X10. In some embodiments, the covalent radionuclide is bound to amino acid X11. In some embodiments, the covalent radionuclide is bound to amino acid X12. In some embodiments, covalent radionuclide is bound to amino acid X2, X6, X7, or X9.

In one aspect, described herein is a radiopharmaceutical conjugate with structure of

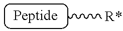

wherein
[symbol] represents the linker; and
R*represents the radionuclide.

In one aspect, described herein is a radiopharmaceutical conjugate with structure of

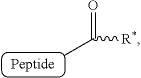

wherein
[symbol] represents the linker connected to the C-terminus of the peptide; and
R* represents the radionuclide.

In one aspect, described herein is a radiopharmaceutical conjugate with structure of

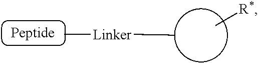

wherein

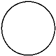

represents the residualizing agent or the non-residualizing agent;
linker represents the linker; and
R* represents the radionuclide.

In one aspect, described herein is a radiopharmaceutical conjugate with structure of

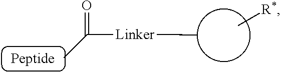

wherein

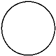

represents the residualizing agent or the non-residualizing agent;
linker represents the linker connected to the C-terminus of the peptide; and
R* represents the radionuclide.

In some embodiments, described herein is a conjugate comprising: (a) a targeting moiety that comprises a monocyclic peptide that has avidity for ephrin type-A receptor 2 (EphA2) and (b)(i) a metal chelator configured to bind with a radionuclide; or (ii) a covalent radionuclide. In some embodiments, described herein is a conjugate comprising: (a) a monocyclic peptide that is configured to bind with EphA2 and (b)(i) a metal chelator configured to bind with a radionuclide; or (ii) a covalent radionuclide. In some embodiments, described herein is a conjugate comprising: (a) a targeting moiety that comprises a monocyclic peptide; and (b)(i) a metal chelator configured to bind with a radionuclide; or (ii) a covalent radionuclide. In some embodiments, the monocyclic peptide is cyclized by a non-disulfide bond. In some embodiments, the monocyclic peptide does not comprise a disulfide bond. In some embodiments, the monocyclic peptide comprises 5 to 20 amino acid residues. In some embodiments, the monocyclic peptide comprises 7 to 12 amino acid residues. A conjugate described herein can further comprises a linker that covalently attaches the cyclic peptide to the metal chelator or the covalent radionuclide. In some embodiments, the conjugate comprises a radionuclide such as ²²⁵Ac bound to the metal chelator. In some embodiments, the conjugate comprises a covalently bound radionuclide such as ¹⁸F, ⁷⁴As, ⁷⁶Br, ¹²³I, ¹²⁴I, ¹²⁵I, ¹³¹I, and ²¹¹At. In some embodiments, the a covalent radionuclide is attached to the peptide or linker via a residualizing agent or the non-residualizing agent.

In some embodiments, a herein-described conjugate comprises two or more peptides (i.e., a first peptide, a second peptide, etc.). For example, the conjugate can comprise two different peptides, wherein both of the peptides are configured to bind to the same target (e.g., EphA2), either at the same binding site or at different binding sites. For another example, the conjugate can comprise two different peptides, wherein the two peptides are configured to bind to different targets (including EphA2). For yet another example, the conjugate can comprise two identical peptides.

In some embodiments, a herein-described conjugate is in a salt form. In some embodiments, a herein-described conjugate is in a free-base form.

In one aspect, described herein is a conjugate comprising (a) a peptide that has avidity for ephrin type-A receptor 2 (EphA2), wherein the peptide competes for binding to human EphA2 with a peptide that has an amino acid sequence including deletion, substitution, and/or addition of one or several amino acids in the amino acid of SEQ ID NO: 1:

(SEQ ID NO: 1)
da-MeF-N-L-Hgl-MeF-W1Me-V-W1Me-T-E-C

or a pharmaceutically acceptable salt thereof; and (b)(i) a metal chelator configured to bind with a radionuclide; or (ii) a covalent radionuclide. In one aspect, described herein is a conjugate comprising (a) a peptide that has avidity for ephrin type-A receptor 2 (EphA2), wherein the peptide competes for binding to human EphA2 with a peptide that has a structure of Formula (I) as described herein (e.g., Formulas (I-1), (I-2), (I-3) or (I-4)), or a pharmaceutically acceptable salt thereof; and (b)(i) a metal chelator configured to bind with a radionuclide; or (ii) a covalent radionuclide. In some embodiments, the peptide competes for binding to human EphA2 at one or more amino acid residues selected from Asp53, Met55, Asn57, Met59, Met66, Thr101, Arg103, Phe156, Glu157, Arg159, Val161, Val189, and Ala190. In some embodiments, the peptide competes for binding to human EphA2 at one or more amino acid residues selected from Asp53, Phe156, and Glu157. In some embodiments, the peptide competes for binding to human EphA2 at Asp53, Glu157, or both. In some embodiments, the peptide is a cyclic peptide. In some embodiments, the peptide is a monocyclic peptide.

In some embodiments, the metal chelator is conjugated to the peptide, either directly or indirectly through a linker. In some embodiments, the metal chelator is conjugated to the peptide, either covalently or non-covalently. In some embodiments, the radionuclide is covalently bound to the peptide, either directly or indirectly through a linker.

A conjugate described herein can have a suitable plasma half-life (T_{1/2}). In some embodiments, the plasma half-life of a conjugate is at least 50 minutes, 100 minutes, 150 minutes, 200 minutes, 250 minutes, 300 minutes, 350 minutes, 400 minutes, 450 minutes, or 500 minutes as determined in vitro in human plasma at 37 °C. In some embodiments, the plasma half-life of a conjugate is at least 280 minutes as determined in vitro in human plasma at 37 °C. In some embodiments, the plasma half-life of a conjugate is at least 250 minutes as determined in vitro in human plasma at 37 °C. In some embodiments, the plasma half-life of a conjugate is at most 30 days, 14 days, 7 days, 2 days, 1 day or 500 minutes as determined in vitro in human plasma at 37 °C. In some embodiments, the plasma half-life is at least 50 minutes, 100 minutes, 150 minutes, 200 minutes, 250 minutes, 300 minutes, 350 minutes, 400 minutes, 450 minutes, or 500 minutes as determined in vivo in a human. In some embodiments, the plasma half-life is at least 280 minutes as determined in vivo in a human. In some embodiments, the plasma half-life is at least 250 minutes as determined in vivo in a human. In some embodiments, the plasma half-life of a conjugate is at most 30 days, 14 days, 7 days, 2 days, 1 day or 500 minutes as determined in vivo in a human. In some embodiments, the plasma half-life of a conjugate is at least 50 minutes, 100 minutes, 150 minutes, 200 minutes, 250 minutes, 300 minutes, 350 minutes, 400 minutes, 450 minutes, or 500 minutes as determined in vitro in a mouse plasma at 37 °C. In some embodiments, the plasma half-life of a conjugate is at least 280 minutes as determined in vitro in a mouse plasma at 37 °C. In some embodiments, the plasma half-life of a conjugate is at least 250 minutes as determined in vitro in a mouse plasma at 37 °C. In some embodiments, the plasma half-life of a conjugate is at most 30 days, 14 days, 7 days, 2 days, 1 day or 500 minutes as determined in vitro in mouse plasma at 37 °C. In some embodiments, the plasma half-life is at least 50 minutes, 100 minutes, 150 minutes, 200 minutes, 250 minutes, 300 minutes, 350 minutes, 400 minutes, 450 minutes, or 500 minutes as determined in vivo in a mouse. In some embodiments, the plasma half-life is at least 280 minutes as determined in vivo in a mouse. In some embodiments, the plasma half-life is at least 250 minutes as determined in vivo in a mouse. In some embodiments, the plasma half-life of a conjugate is at most 30 days, 14 days, 7 days, 2 days, 1 day or 500 minutes as determined in vivo in a mouse. Plasma half-life can be determined by any suitable methods known in the art, e.g., the method described in Example C1. In some embodiments, the conjugate has a plasma half-life (T_{1/2}) of at least 250 minutes as determined in vitro in human plasma at 37 °C. In some embodiments, plasma half-life is determined by % remaining of test compound after incubation in plasma.

A conjugate described herein can have a an uptake ratio between a tumor and intestine. In some embodiments, an uptake ratio is determined between the uptake of a radiopharmaceutical conjugate to a tumor and the uptake of a radiopharmaceutical conjugate to a kidney of a subject. In some embodiments, the subject is a human. In some embodiments, is a mammal. In some embodiments, the subject is a rat or mouse (such as in a xenograft model). In some embodiments, an uptake ratio between a tumor uptake and kidney uptake (i.e., tumor uptake/kidney uptake) toward the radiopharmaceutical conjugate is at least 1.0, 1.1, 1.2, 1.3, 1.4, 1.5, 1.6, 1.7, 1.8 or 2.0 in a human prostate xenograft mouse model.). In some embodiments, the uptake ratio is determined at about 4 hours, 12 hours, 24 hours, or 48 hours after administration of the radiopharmaceutical conjugate to the mouse. In some embodiments, an uptake ratio between a tumor uptake and kidney uptake toward the radiopharmaceutical conjugate is at least 1.2. In some embodiments, an uptake ratio between a tumor uptake and kidney uptake toward the radiopharmaceutical conjugate is at least 1.5. In some embodiments, a tumor uptake of a herein described radiopharmaceutical conjugate is at least 5%, 10%, 20%, 30%, 40%, 50%, 60%, 70%, 80%, 90%, or 100% higher than a kidney uptake of the radiopharmaceutical conjugate in a same subject.

In some embodiments, the uptake of the radiopharmaceutical conjugate is determined at about 4 hours, 12 hours, 24 hours, or 48 hours after administration of the radiopharmaceutical conjugate to the subject. In some embodiments, the uptake of the radiopharmaceutical conjugate is determined at about 4 hours after administration. In some embodiments, the uptake of the radiopharmaceutical conjugate is determined at about 12 hours after administration. In some embodiments, the uptake of the radiopharmaceutical conjugate is determined at about 24 hours. In some embodiments, the uptake of the radiopharmaceutical conjugate is determined at about 48 hours after administration.

In some embodiments, a conjugate described herein is designed to have a prescribed elimination profile. The elimination profile can be designed by adjusting the sequence and length of the peptide, the property of the linker, the type of radionuclide, etc. In some embodiments, the conjugate has an elimination half-life of about 30 minutes to 120 hours. In some embodiments, the conjugate has an elimination half-life of about 1 to 120 hours. In some embodiments, the conjugate has an elimination half-life of at least 15 minutes, 30 minutes, 1 hour, 2 hours, 3 hours, 4 hours, 5 hours, 7 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some embodiments, the conjugate has an elimination half-life of at most 120 hour, 80 hours, 70 hours, 60 hours, 50 hours, 40 hours, 30 hours, 24 hours, 12 hours, 10 hours, or 5 hours. In some embodiments, the conjugate has an elimination half-life of about 2 to 24 hours. In some embodiments, the conjugate has an elimination half-life of about 3 to 9 hours. In some embodiments, the conjugate has an elimination half-life of about 2 to 12 hours. In some embodiments, the conjugate has an elimination half-life of about 2 to 8 hours. In some embodiments, the conjugate has an elimination half-life of about 2 to 5 hours. In some embodiments, the conjugate has an elimination half-life of about 3 to 4 hours. In some embodiments, the elimination half-life is determined in rats. In some embodiments, the elimination half-life is determined in humans.

A herein described conjugate can have an elimination half-life in a tumor and non-tumor tissue of the subject. The elimination half-life in a tumor can be the same as or different from (either longer or shorter than) the elimination half-life in a non-tumor issue. In some embodiments, the elimination half-life of the conjugate in a tumor is about 3 hours to 14 days, about 2 to 10 days, about 7 to 10 days, or about 4 to 7 days. In some embodiments, the elimination half-life of the conjugate in a tumor is more than 14 days. In some embodiments, the elimination half-life of the conjugate in a non-tumor tissue is about 1 hour to 14 days, about 12 hours to 2 days, about 1 day to 3 days, about 2 to 10 days, about 7 to 10 days, or about 4 to 7 days. In some embodiments, the elimination half-life of the conjugate in a tumor is at least 1.1, 1.2, 1.3, 1.4, 1.5, 2.0, 2.5, 3.0, 4.0, or 5.0 fold of the elimination half-life of the conjugate in a non-tumor tissue of the subject.

As used herein, the “elimination half-life” can refer to the time it takes from the maximum concentration after administration to half maximum concentration. In some embodiments, the elimination half-life is determined after intravenous administration. In some embodiments, the elimination half-life is measured as biological half-life, which is the half-life of the cold pharmaceutical in the living system. In some embodiments, the elimination half-life is measured as effective half-life, which is the half-life of a radiopharmaceutical in a living system taking into account the half-life of the radionuclide.

In some cases, the elimination profile of the conjugate can be adjusted by a reversible binding between the conjugate and a plasma protein such as albumin. A suitable affinity between the conjugate and the plasma protein can utilize the plasma protein as a reservoir for the conjugates, attaching and preserving the conjugates at high concentration and releasing the conjugates at a lower concentration, thereby improving elimination profile. In some embodiments, a dissociation constant (Kd) between the conjugate and human serum albumin is at most 500 μM, as determined at room temperature in human serum condition. In some embodiments, the Kd is from about 0.1 nM to about 1000 μM. In some embodiments, the Kd is at most 100 μM. In some embodiments, the Kd is at most 15 μM. In some embodiments, the Kd is from about 1 nM to about 10 μM. In some embodiments, the Kd is from about 10 nM to about 10 μM. In some embodiments, the Kd is from about 50 nM to about 1 μM. In some embodiments, the Kd is from about 100 nM to about 10 μM.

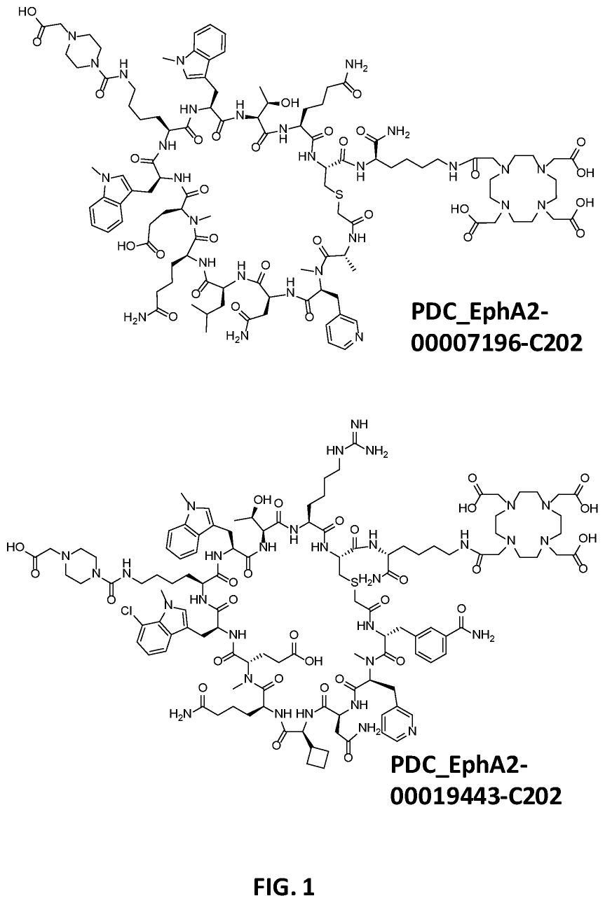

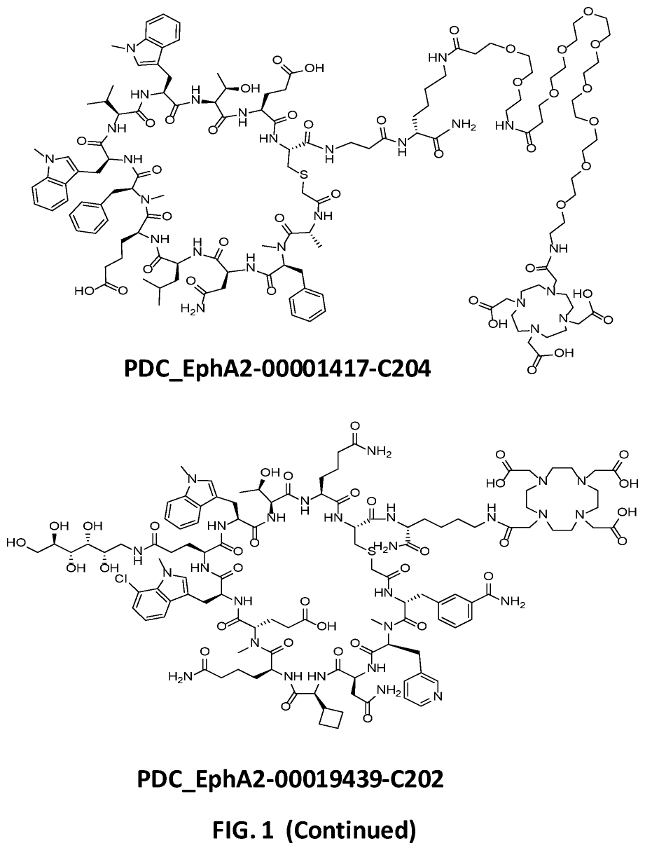

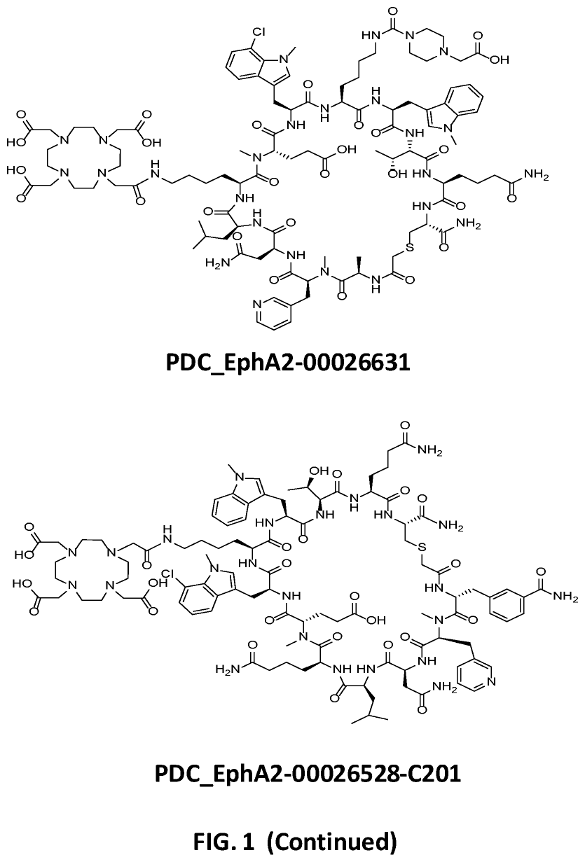

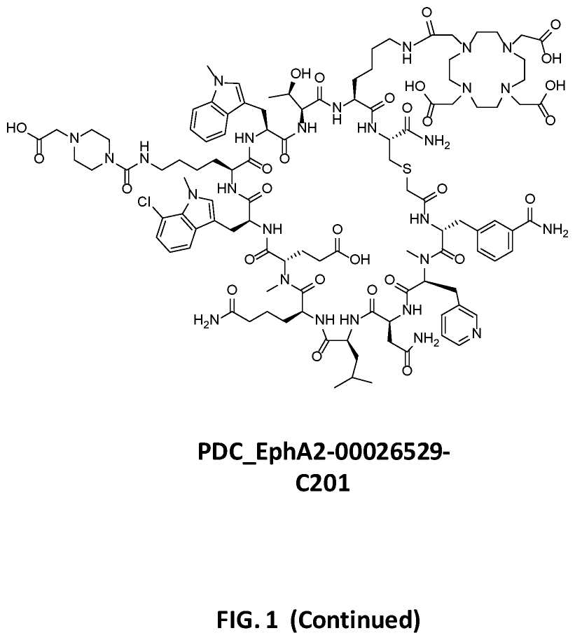

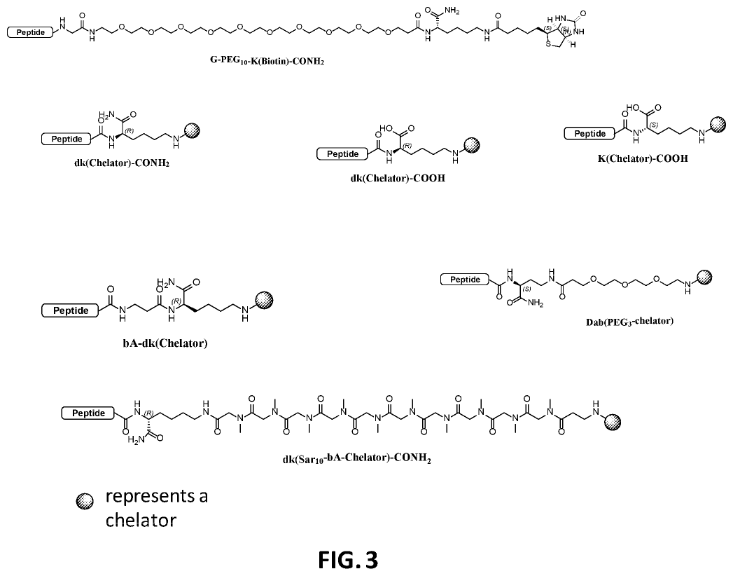

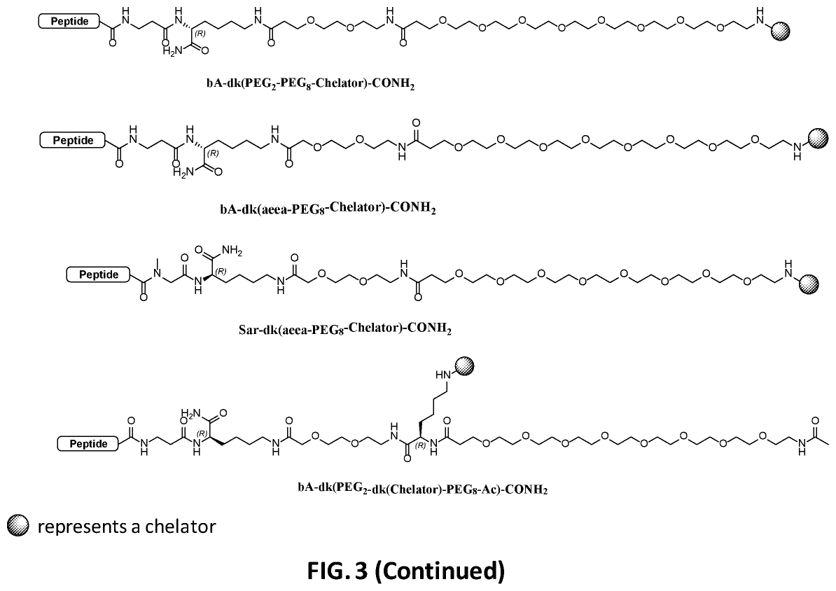

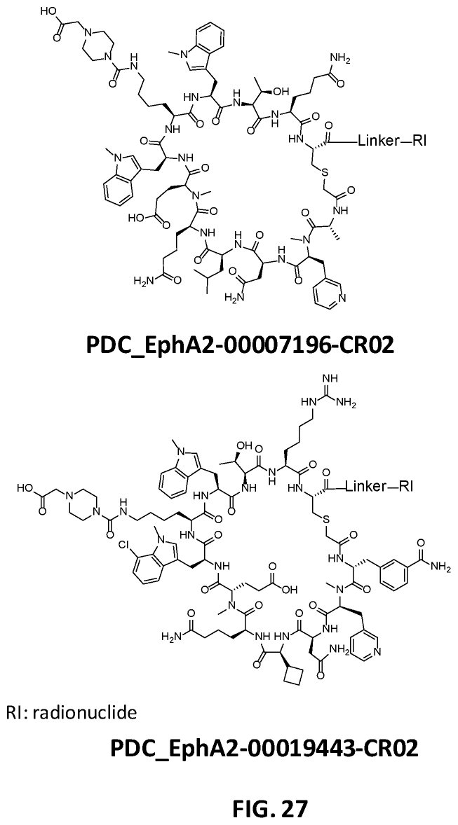

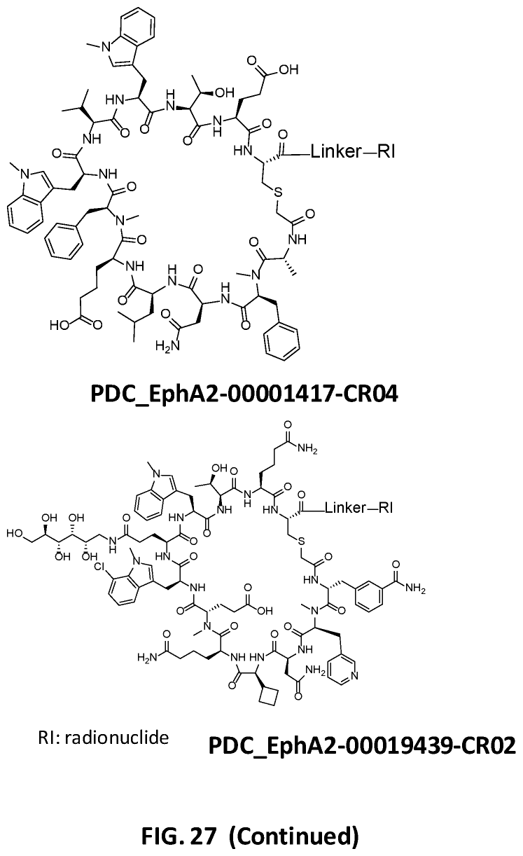

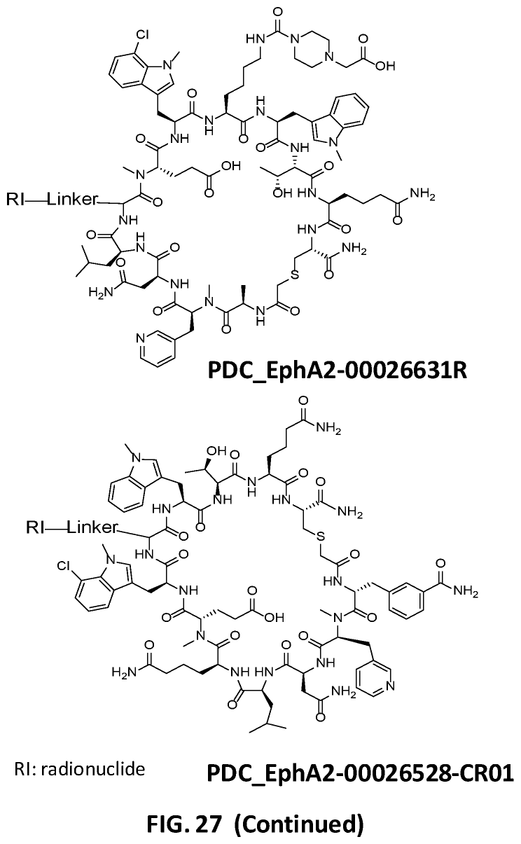

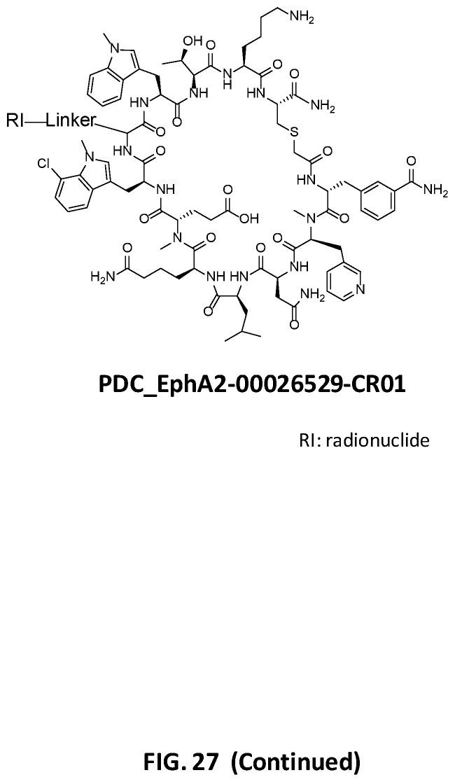

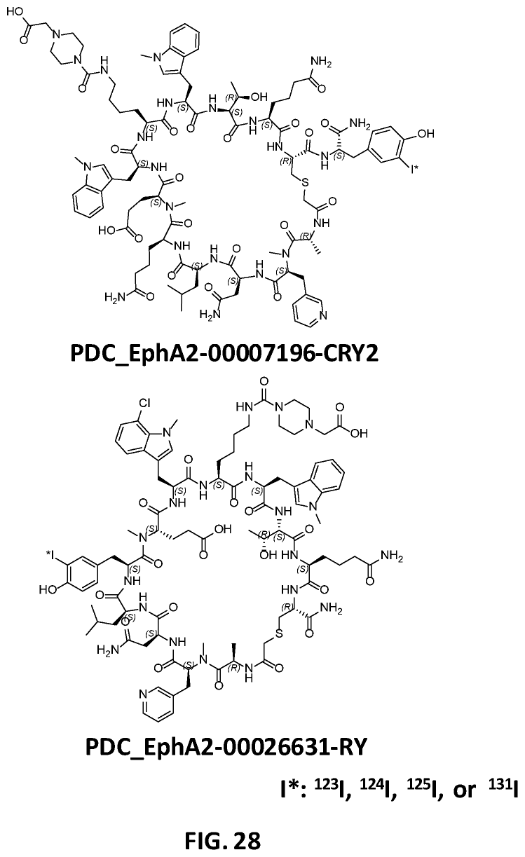

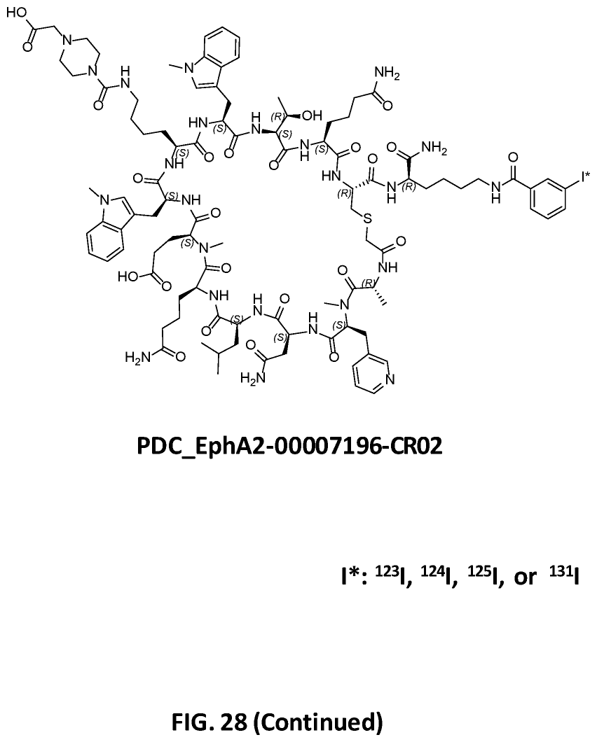

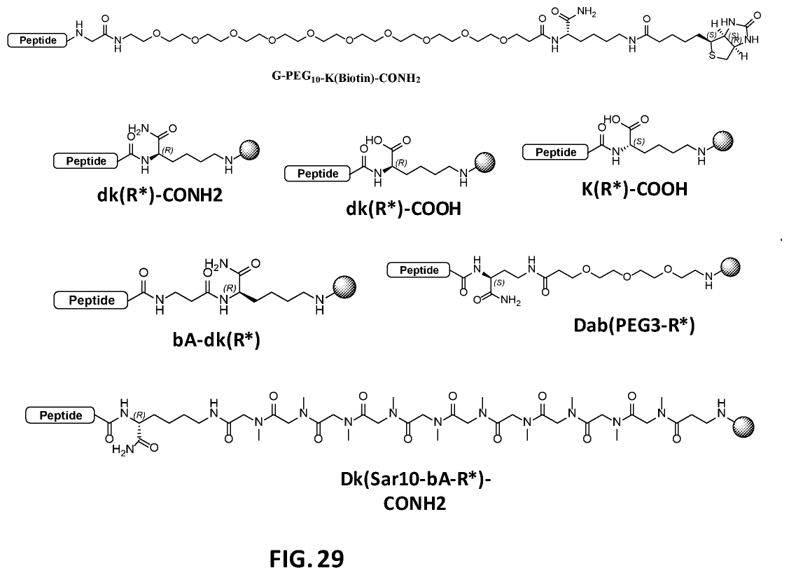

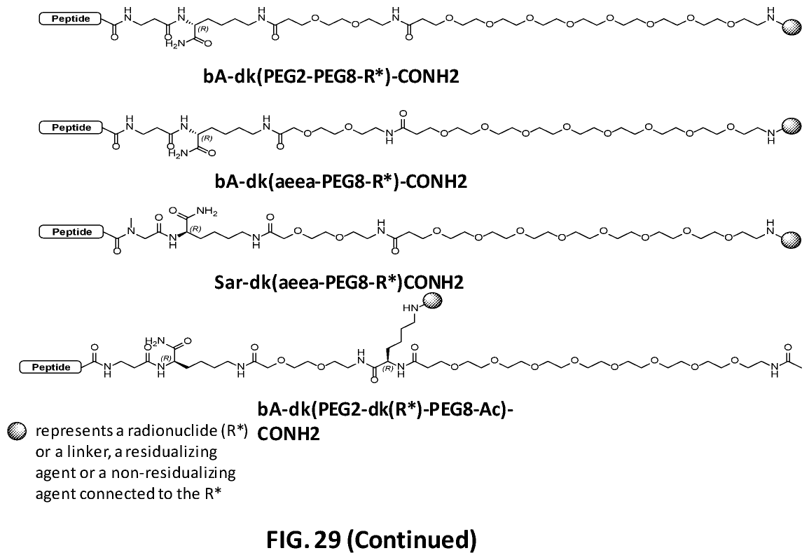

In some embodiments, a conjugate of the present disclosure is selected from Tables 2A-Lu, 2A-Lu177, 2A-Ac255, 2B, 2BLu, 2B-Lu177, 2B-Ac255, and 2C. In some embodiments, a conjugate of the present disclosure is selected from Tables 2A-Ac255, and 2B-Ac255. In some embodiments, a conjugate of the present disclosure comprises a peptide of Table 1, a chelator selected from FIGS. 4-22, and a radionuclide of Table 7 labeled “chelator”. In some embodiments, a conjugate of the present disclosure comprises a conjugate of FIG. 1-3. In some embodiments, a conjugate of the present disclosure comprises a peptide of Table 1 and a radionuclide of Table 7 labeled “covalent”. In some embodiments, a conjugate of the present disclosure comprises a peptide of Table 1, a linker, and a radionuclide of Tables 7 marked “covalent”. In some embodiments, a conjugate of the present disclosure comprises a conjugate of FIG. 27-29.

### EphA2

EPH receptor A2 (ephrin type-A receptor 2) is a protein that in humans is encoded by the EPHA2 gene. EphA2 may be upregulated in multiple cancers, often correlating with disease progression, metastasis and poor prognosis e.g., in solid tumors such as breast, lung, gastric, pancreatic, prostate, liver and glioblastoma.

Eph receptor tyrosine kinases (Ephs) belong to a large group of receptor tyrosine kinases (RTKs), kinases that phosphorylate proteins on tyrosine residues. Ephs and their membrane bound ephrin ligands (ephrins) can control cell positioning and tissue organization. Functional and biochemical Eph responses can occur at higher ligand oligomerization states.

Among other patterning functions, various Ephs and ephrins have been shown to play a role in vascular development. Knockout of EphB4 and ephrin-B2 can result in a lack of the ability to remodel capillary beds into blood vessels and embryonic lethality. Persistent expression of some Eph receptors and ephrins has also been observed in newly-formed, adult micro-vessels (Brantley-Sieders et al. (2004) Curr Pharm Des 10, 3431-42). The de-regulated re-emergence of some ephrins and their receptors in adults may contribute to tumor invasion, metastasis and neo-angiogenesis. Furthermore, some Eph family members may be over-expressed on tumor cells from a variety of human tumors (Booth et al. (2002) Nat Med 8, 1360-1).

Human EphA2 can have a sequence according to the following Seq ID NO: 276
(Isoform 1, P29317-1):
(SEQ ID NO: 276)
MELQAARACFALLWGCALAAAAAAQGKEVVLLDFAAAGGELGWLTHPYGKGWDLMQNIMN
DMPIYMYSVCNVMSGDQDNWLRTNWVYRGEAERIFIELKFTVRDCNSFPGGASSCKETFNLYY
AESDLDYGTNFQKRLFTKIDTIAPDEITVSSDFEARHVKLNVEERSVGPLTRKGFYLAFQDIGACV
ALLSVRVYYKKCPELLQGLAHFPETIAGSDAPSLATVAGTCVDHAVVPPGGEEPRMHCAVDGE
WLVPIGQCLCQAGYEKVEDACQACSPGFFKFEASESPCLECPEHTLPSPEGATSCECEEGFFRAP
QDPASMPCTRPPSAPHYLTAVGMGAKVELRWTPPQDSGGREDIVYSVTCEQCWPESGECGPCE
ASVRYSEPPHGLTRTSVTVSDLEPHMNYTFTVEARNGVSGLVTSRSFRTASVSINQTEPPKVRLE
GRSTTSLSVSWSIPPPQQSRVWKYEVTYRKKGDSNSYNVRRTEGFSVTLDDLAPDTTYLVQVQA
LTQEGQGAGSKVHEFQTLSPEGSGNLAVIGGVAVGVVLLLVLAGVGFFIHRRRKNQRARQSPED
VYFSKSEQLKPLKTYVDPHTYEDPNQAVLKFTTEIHPSCVTRQKVIGAGEFGEVYKGMLKTSSG
KKEVPVAIKTLKAGYTEKQRVDFLGEAGIMGQFSHHNIIRLEGVISKYKPMMIITEYMENGALDK
FLREKDGEFSVLQLVGMLRGIAAGMKYLANMNYVHRDLAARNILVNSNLVCKVSDFGLSRVLE
DDPEATYTTSGGKIPIRWTAPEAISYRKFTSASDVWSFGIVMWEVMTYGERPYWELSNHEVMK
AINDGFRLPTPMDCPSAIYQLMMQCWQQERARRPKFADIVSILDKLIRAPDSLKTLADFDPRVSI
RLPSTSGSEGVPFRTVSEWLESIKMQQYTEHFMAAGYTAIEKVVQMINDDIKRIGVRLPGHQKRI
AYSLLGLKDQVNTVGIPI.
Human EphA2 can have a sequence according to the following Seq ID NO: 277
(Isoform 2, P29317-2):
(SEQ ID NO: 277)
MELQAARACFALLWGCALAAAAAAQGKEVVLLDFAAAGGELGWLTHPYGKGWDLMQNIMN
DMPIYMYSVCNVMSGDQDNWLRTNWVYRGEAERIFIELKFTVRDCNSFPGGASSCKETFNLYY
AESDLDYGTNFQKRLFTKIDTIAPDEITVSSDFEARHVKLNVEERSVGPLTRKGFYLAFQDIGACV
ALLSVRVYYKKCPELLQGLAHFPETIAGSDAPSLATVAGTCVDHAVVPPGGEEPRMHCAVDGE
WLVPIGQCLCQAGYEKVEDACQACSPGFFKFEASESPCLECPEHTLPSPEGATSCECEEGFFRAP
QDPASMPCTRPPSAPHYLTAVGMGAKVELRWTPPQDSGGREDIVYSVTCEQCWPESGECGPCE
ASVRYSEPPHGLTRTSVTVSDLEPHMNYTFTVEARNGVSGLVTSRSFRTASVSINQTEPPKVRLE
GRSTTSLSVSWSIPPPQQSRVWKYEVTYRKKVTPRGAGLALAGPTAGDRLVT.

As used herein, the expression “has avidity for EphA2” or “binds to EphA2” indicates having the activity of binding to EphA2. Binding site of the peptide of the present disclosure on the EphA2 is not limited, the peptide can bind to anywhere on the EphA2 protein. Binding to EphA2 may be measured by any method for measuring known intermolecular binding. In a non-limiting manner, for example, this may be determined by competitive binding assays such as surface plasmon resonance (SPR) assays, scatter analysis and/or radioimmunoassays (RIA), enzyme immunoassays (EIA), and sandwich and competitive assays, and in any suitable manner which is known, including different variants of the examples given that are known in the technical field.

In some embodiments, a peptide or a radiopharmaceutical conjugate comprising the peptide binds to EphA2. In some embodiments, the peptide or conjugate has EphA2 antagonistic activity. In some embodiments, the peptide or conjugate binds to human EphA2 (hEphA2) and has hEphA2 antagonistic activity.

As used herein, the term “EphA2” refers to any form of EphA2 and a variant thereof for retaining at least a part of the activity of EphA2. The EphA2 includes all the native sequences of EphA2 in mammals such as, for example, humans, dogs, cats, horses, and cows, unless otherwise specifically described as human EphA2 (hEphA2). One exemplification of EphA2 is hEphA2 (Gene ID: 1969), which is human EphA2 and is a protein having an amino acid sequence (SEQ ID NO: 276, Isoform 1, P29317-1).

### Peptide Ligand

In one aspect, a conjugate described herein comprises a peptide (e.g., a binding peptide) that has avidity for ephrin type-A receptor 2 (EphA2). The EphA2 can be a mammalian EphA2. The EphA2 can be a human EphA2. The EphA2 can be a wild-type or mutated EphA2. In some embodiments, the conjugate comprises two or more peptides, which can be the same or different. The peptide can be linear or cyclic. In some embodiments, the peptide is monocyclic. The peptide can comprise any suitable number of amino acid residues. In some embodiments, the peptide comprises from 5 to 50, 6 to 40, 7 to 30, 8 to 25, 12 to 25, or 9 to 20 amino acid residues. In some embodiments, the peptide comprises from 5 to 14 amino acid residues. In some embodiments, the peptide comprises from 7 to 12 amino acid residues. In some embodiments, the peptide comprises from 8 to 12 amino acid residues. In some embodiments, the peptide comprises from 8 to 10 amino acid residues. In some embodiments, the peptide comprises from 7 to 13 amino acid residues. In some embodiments, the peptide comprises from 12 to 15 amino acid residues. In some embodiments, the peptide comprises from 13 to 14 amino acid residues. In some embodiments, the peptide comprises 6 amino acid residues. In some embodiments, the peptide comprises 7 amino acid residues. In some embodiments, the peptide comprises 8 amino acid residues. In some embodiments, the peptide comprises 9 amino acid residues. In some embodiments, the peptide comprises 10 amino acid residues. In some embodiments, the peptide comprises 11 amino acid residues. In some embodiments, the peptide comprises 12 amino acid residues. In some embodiments, the peptide comprises 13 amino acid residues. In some embodiments, the peptide comprises 14 amino acid residues. In some embodiments, the peptide comprises 15 amino acid residues. In some embodiments, the peptide comprises 16 amino acid residues. In some embodiments, the peptide consists of 6 amino acid residues. In some embodiments, the peptide consists of 7 amino acid residues. In some embodiments, the peptide consists of 8 amino acid residues. In some embodiments, the peptide consists of 9 amino acid residues. In some embodiments, the peptide consists of 10 amino acid residues. In some embodiments, the peptide consists of 11 amino acid residues. In some embodiments, the peptide consists of 12 amino acid residues. In some embodiments, the peptide consists of 13 amino acid residues. In some embodiments, the peptide consists of 14 amino acid residues. In some embodiments, the peptide consists of 15 amino acid residues. In some embodiments, the peptide consists of 16 amino acid residues. In some embodiments, the conjugate comprises a monocyclic peptide of 6, 7, 8, 9, 10, 11, 12, 13, 14, or 15 amino acid residues. A peptide described herein can be a binding peptide that binds to EphA2. In some embodiments, the binding peptide consists of 6 to 20 amino acid residues. In some embodiments, the binding peptide consists of 7 to 12 amino acid residues. In some embodiments, the binding peptide consists of 10 to 12 amino acid residues. In some embodiments, the binding peptide consists of 8 to 12 amino acid residues. In some embodiments, the binding peptide is monocyclic. In some embodiments, the peptide of the present technology is an isolated peptide. In some embodiments, the peptide of the present technology is a purified peptide.

In one aspect, described herein is a peptide (e.g., a cyclic peptide) that has avidity for ephrin type-A receptor 2 (EphA2), wherein the peptide comprises an amino acid sequence including deletion, substitution, and/or addition of one or several (e.g., 1-6) amino acids in the amino acid of SEQ ID NO:1:

(SEQ ID NO: 1)
da-MeF-N-L-Hgl-MeF-W1Me-V-W1Me-T-E-C

or a pharmaceutically acceptable salt thereof.

In some embodiments, the (cyclic) peptide consists of 10 to 12 amino acid residues.

In some embodiments, the peptide comprises an amino acid sequence including a total of at most 6 deletion, substitution, and/or addition of one or several amino acids in the amino acid of SEQ ID NO:1. In some embodiments, the peptide comprises an amino acid sequence including a total of at most 5 deletion, substitution, and/or addition of one or several amino acids in the amino acid of SEQ ID NO:1. In some embodiments, the peptide comprises an amino acid sequence including a total of at most 4 deletion, substitution, and/or addition of one or several amino acids in the amino acid of SEQ ID NO:1. In some embodiments, the peptide comprises an amino acid sequence including a total of at most 3 deletion, substitution, and/or addition of one or several amino acids in the amino acid of SEQ ID NO: 1. In some embodiments, the peptide comprises an amino acid sequence including a total of at most 2 deletion, substitution, and/or addition of one or several amino acids in the amino acid of SEQ ID NO:1. In some embodiments, the peptide comprises an amino acid sequence including a total of at most 1 deletion, substitution, and/or addition of one or several amino acids in the amino acid of SEQ ID NO:1. In some embodiments, the amino acid substitution is a conservative amino acid substitution. The deletion, addition, or substitution position may be either the end or middle of the peptide. In some embodiments, 1-5 amino acids selected from the group consisting of 3^{rd} N, 4^{th} L, 6^{th} MeF, 10^{th} T and 11^{th} E of SEQ ID NO: 1 is/are deleted, optionally without additional addition and/or substitution. In some embodiments, one to several (e.g., 1, 2, 3, 4 or 5) amino acids are added. In some embodiments, one or more amino acid residues selected from the 2^{nd} MeF, 6th MeF, 8^{th} V and 11^{th} E are substituted. In some embodiments, the peptide comprises an amino acid sequence with deletion of 2 or less amino acids in the amino acid SEQ ID NO: 1, optionally without additional addition and/or substitution. In some embodiments, 1-2 amino acids selected from the group consisting of 10th T and 11th E of SEQ ID NO:1 is/are deleted, optionally without additional addition and/or substitution. In some embodiments, the 8th V is substituted. In some embodiments, the 11th E is substituted.

For the purpose of the disclosure, one event of “substitution” of an amino acid or an amino sequence is not considered two separate events of one deletion plus one addition. Thus, for the avoidance of doubt, as an example, a sequence change of “up to two deletion, substitution and/or addition” includes one deletion and one substitution, one deletion and one addition (at a different position), one substitution and one addition, one deletion only, one substitution only, one addition only, two deletions, two substitutions, two additions, etc. The deletion, addition, or substitution position may be at one or both ends of the peptide, or in the middle of the peptide.

In some embodiments, the peptide comprises an amino acid sequence wherein 1-5 amino acids selected the group consisting of third N, 4th L, 5th Hgl, 6th MeF, 10th T and 11th E of SEQ ID NO: 1, is deleted in the peptide. In some embodiments, the peptide comprises an amino acid sequence wherein 1, 2, 3, 4 or 5 amino acids selected the group consisting of third N, 4th L, 5th Hgl, 6th MeF, 10th T and 11th E of SEQ ID NO: 1, is deleted in the peptide. In some embodiments, third N is deleted. In some embodiments, 4th L is deleted. In some embodiments, 5th Hgl is deleted. In some embodiments, 6th MeF is deleted. In some embodiments, 11th E is deleted. In some embodiments, the peptide comprises an amino acid sequence wherein 1-5 amino acids selected from the group consisting of amino acids at the 3^{rd}, 4^{th}, 5^{th}, 6^{th}, 10^{th}, and 11^{th} position of SEQ ID NO: 1, is deleted in the peptide. In some embodiments, the peptide comprises an amino acid sequence wherein 1, 2, 3, 4 or 5 amino acids selected the group consisting of amino acids at the 3^{rd}, 4^{th}, 5^{th}, 6^{th}, 10^{th}, and 11^{th} position of SEQ ID NO: 1, is deleted in the peptide. In some embodiments, 3^{rd} amino acid is deleted. In some embodiments, the 4^{th} amino acid is deleted. In some embodiments, the 5^{th} amino acid is deleted. In some embodiments, the 6^{th} amino acid is deleted. In some embodiments, the 10^{th} amino acid is deleted. In some embodiments, the 11^{th} amino acid is deleted. In certain embodiments, the peptide has deletions of 1-5 amino acids of SEQ ID NO: 1, and no additional residue addition. In certain embodiments, the peptide has deletions of 1-5 amino acids of SEQ ID NO: 1, and no additional residue substitutions. In certain embodiments, the peptide has deletions of 1-5 amino acids of SEQ ID NO: 1, and no additional residue addition or substitution. In certain embodiments, the peptide has deletions of −1-5 amino acid residues of SEQ ID NO: 1, and no residue addition. In certain embodiments, the peptide has deletions of 1-5 amino acid residues of SEQ ID NO: 1 and no residue substitution. In certain embodiments, the peptide has deletions of 1-5 amino acid residues of SEQ ID NO: 1, and no residue addition and substitution.

In one aspect, described herein is a peptide that has avidity for ephrin type-A receptor 2 (EphA2), wherein the peptide has an amino acid sequence according to Formula (I), or a pharmaceutically acceptable salt thereof,

X1-X2-X3-X4-X5-X6-X7-X8-X9-X10-X11-X12 Formula (I)
wherein,
   X1 is an amino acid;
   X2 is an amino acid comprising an aromatic ring, an N-methylated amino acid thereof, or a variant thereof;
   X3 is a hydrophilic amino acid (e.g. N, Q, Cit, K or a variant thereof), glycine (G), Alanine (A) or a variant thereof (e.g., da, 2-Aminoisobutyric acid (Aib));
   X4 is a hydrophobic amino acid (e.g., leucine (L)), a hydrophilic amino acid (e.g., citrulline (Cit)), or a variant thereof;
   X5 is a hydrophilic amino acid, or a variant thereof;
   X6 is a hydrophilic amino acid, an amino acid comprising an aromatic ring, or an N-methylated amino acid thereof;
   X7 is an amino acid comprising an aromatic ring (e.g., W, F, or a variant thereof);
   X8 is a hydrophobic amino acid, a hydrophilic amino acid, an N-methylated amino acid, or a variant thereof;
   X9 is an amino acid comprising an aromatic ring (e.g., W or a variant thereof);
   X10 is absent or a hydrophilic amino acid (e.g., Threonine (T) or a variant thereof);
   X11 is absent or a hydrophilic amino acid; and
   X12 is cysteine (C) or a variant thereof.

In some embodiments of Formula (I), both X10 and X11 are present. In some embodiments of Formula (I), both X10 and X11 are absent.

In one aspect, described herein is a peptide that has avidity for ephrin type-A receptor 2 (EphA2), wherein the peptide has an amino acid sequence according to Formula (I), or a pharmaceutically acceptable salt thereof,

X1-X2-X3-X4-X5-X6-X7-X8-X9-X10-X11-X12 Formula (I)
wherein,
X1 is an amino acid;
X2 is F, or a variant thereof that replaces the unsubstituted phenyl ring of F with
   (i) a phenyl ring substituted by 1 or 2 substituents each independently selected from —OH, —CN, amino, halogen, —C1-3 haloalkyl, and —C1-3 alkyl (e.g., —CH₃), or
   (ii) a 6-membered heteroaryl ring optionally substituted by 1 or 2 substituents each independently selected from —OH, —CN, amino, halogen, —C1-3 haloalkyl, and —C1-3 alkyl, wherein the F or the variant thereof is optionally N-methylated;
X3 is a hydrophilic amino acid (e.g. N, Q, Cit, K or a variant thereof), G, Aib, Hgn, Ala, or a variant thereof (e.g., da);
X4 is a hydrophobic amino acid (e.g., an amino acid having 4 or more carbon atoms in a side chain comprising a linear, branched, or cyclic carbon chain), and wherein X4 is optionally N-methylated (e.g., Cit or a variant thereof);
X5 is an amino acid (e.g., a hydrophilic amino acid; or an amino acid with a functional side chain (e.g., not glycine));
X6 is an N-methylated amino acid thereof;
X7 is a W, Y, or a variant thereof (e.g., an amino acid having either a 6-membered aryl or heteroaryl, or a 9- or 10-membered bi-cyclic aryl or heteroaryl linked to the alpha-carbon through a carbon (e.g., a methylene group)), wherein the 6-, 9-, and 10-membered heteroaryl has 1-3 heteroatoms (e.g., N), and wherein the 6-, 9-, and 10-membered aryl or heteroaryl is optionally substituted (e.g., optionally substituted by 1-4 substituents independently selected from —OH, —CN, amino, halogen, —C1-3 haloalkyl, and —C1-3 alkyl);
X8 is an amino acid with —H on the alpha-amino group (e.g., X8 is not an N-alkylated amino acid);
X9 is W or Y or a variant thereof; (e.g., W or a variant thereof);
X10 is absent, or a polar amino acid (e.g., T or a variant thereof);
X11 is absent or an amino acid (e.g., a hydrophilic amino acid; Dab, Dap, R, E or a variant thereof; or an amino acid with a functional side chain (e.g., not glycine)); and
X12 is C or a variant thereof.

In some embodiments, X2 is F, or a variant thereof that replaces the unsubstituted phenyl ring of F with a phenyl ring substituted by 1 or 2 substituents each independently selected from —OH, —CN, and —C₁₋₃ alkyl (e.g., —CH₃). In some embodiments, X2 is F, or a variant thereof that replaces the unsubstituted phenyl ring of F with a 6-membered heteroaryl ring optionally substituted by 1 or 2 substituents each independently selected from —OH, —CN, amino, halogen, —C₁₋₃ haloalkyl, and —C₁₋₃ alkyl. In some embodiments, the F or the variant thereof is optionally N-methylated. In some embodiments, the 6-membered heteroaryl ring is pyridine, pyrimidine, or pyridazine. In some embodiments, the 6-membered heteroaryl ring is pyridine.

In some embodiments, X7 is a W, Y, or a variant thereof (e.g., an amino acid having either a 6-membered aryl or heteroaryl, or a 9- or 10-membered bi-cyclic aryl or heteroaryl linked to the alpha-carbon through a carbon (e.g., a methylene group)), wherein the 6-, 9-, and 10-membered heteroaryl has one heteroatom (e.g., N), and wherein the 6-, 9-, and 10-membered aryl or heteroaryl is optionally substituted by 1 or 2 substituents independently selected from —CH₃, -ethyl, —Cl, and —F).

In one aspect, described herein is a peptide that has avidity for ephrin type-A receptor 2 (EphA2), wherein the peptide has an amino acid sequence according to Formula (I), or a pharmaceutically acceptable salt thereof,

X1-X2-X3-X4-X5-X6-X7-X8-X9-X10-X11-X12 Formula (I)
wherein,
X1 is any amino acid (e.g., D-amino acid);
X2 is an amino acid comprising an aromatic ring or a substitution thereof, N-methylated amino acid, or a substitution thereof;
X3 is absent, N or a substitution thereof;
X4 is absent, any hydrophobic amino acid or a substitution thereof;
X5 is absent, a hydrophilic amino acid or a substitution thereof, or an amino acid with a functional side chain (e.g., Dab, Dap, K);
X6 is absent, a hydrophilic amino acid or amino acid having aromatic ring, N-methylated amino acid thereof, or a substitution thereof;
X7 is W or a substitution thereof;
X8 is V, hydrophilic amino acid or a substitution thereof, an N-methylated amino acid, or an amino acid with a functional side chain;
X9 is W or a substitution thereof;
X10 is absent, T or a substitution thereof;
X11 is absent, any hydrophilic amino acid, or an amino acid with a functional side chain; and
X12 is C or a substitution thereof.

In some embodiments, the peptide has an amino acid sequence according to Formula (I), or a pharmaceutically acceptable salt thereof, wherein
X1 is any amino acid (e.g., D-amino acid);
X2 is an amino acid comprising an aromatic ring or a variant thereof, or N-methylated amino acid thereof;
X3 is absent, N or a variant thereof;
X4 is absent, any hydrophobic amino acid or a variant thereof;
X5 is absent, a hydrophilic amino acid or a variant thereof, or an amino acid with a functional side chain (e.g., Dab, Dap, K);
X6 is absent, a hydrophilic amino acid or amino acid having aromatic ring, or N-methylated amino acid thereof;
X7 is W or a variant thereof;
X8 is V, hydrophilic amino acid or a variant thereof, an N-methylated amino acid, or an amino acid with a functional side chain;
X9 is W or a variant thereof;
X10 is absent, T or a variant thereof;
X11 is absent, any hydrophilic amino acid, or an amino acid with a functional side chain; and
X12 is C or a variant thereof.

In some embodiments, of a peptide of Formula (I), or a pharmaceutically acceptable salt thereof, wherein
X1 is any amino acid;
X2 is an amino acid comprising an aromatic ring, or N-methylated amino acid thereof;
X3 is absent, a hydrophilic amino acid (e.g. N, Q, Cit, K or a variant thereof), G, Aib, Hgn, or Ala or a variant thereof (e.g., da);
X4 is absent, a hydrophobic amino acid, or a hydrophilic amino acid (e.g., Cit or a variant thereof);
X5 is absent, a hydrophilic amino acid, or an amino acid with a functional side chain;
X6 is absent, a hydrophilic amino acid, or an or amino acid having aromatic ring, or N-methylated amino acid thereof;
X7 is an amino acid comprising an aromatic ring (e.g., W or a variant thereof);
X8 is a hydrophobic amino acid, a hydrophilic amino acid, an N-methylated amino acid, or an amino acid with a functional side chain;
X9 is an amino acid comprising an aromatic ring (e.g., W or a variant thereof);
X10 is absent, or a polar amino acid (e.g., T or a variant thereof);
X11 is absent, a hydrophilic amino acid, or an amino acid with a functional side chain; and
X12 is C or a variant thereof.

In some embodiments, of a peptide of Formula (I), or a pharmaceutically acceptable salt thereof, wherein
X1 is an amino acid (e.g., D-amino acid);
X2 is an amino acid comprising an aromatic ring, or N-methylated amino acid thereof;
X3 is absent, a hydrophilic amino acid (e.g. N, Q, Cit, K or a variant thereof), G, Aib,
Hgn, or Ala or a variant thereof (e.g, da);
X4 is a hydrophobic amino acid, or a hydrophilic amino acid (e.g., Cit or a variant thereof);
X5 is a hydrophilic amino acid (e.g., Dab, Dap, R, E or a variant thereof);
X6 is absent, a hydrophilic amino acid, an amino acid having aromatic ring (e.g., W), or
N-methylated amino acid thereof;
X7 is an amino acid comprising an aromatic ring (e.g., W or a variant thereof);
X8 is a hydrophobic amino acid, a hydrophilic amino acid, or an N-methylated amino acid;
X9 is an amino acid comprising an aromatic ring (e.g., W or a variant thereof);
X10 is absent, or a hydrophilic amino acid (e.g., T or a variant thereof);
X11 is absent, or a hydrophilic amino acid; and
X12 is C or a variant thereof.

In some embodiments, of a peptide of Formula (I), or a pharmaceutically acceptable salt thereof, wherein
X1 is an amino acid (e.g., D-amino acid);
X2 is F or a variant thereof, Y or a variant thereof, or W or a variant thereof, or N-methylated amino acid thereof;
X3 is absent, N, Q, Cit or a variant thereof, G, Aib, Hgn, K or a variant thereof, Ala, or da;
X4 is absent, G substituted with straight or branched C₁₋₅ alkyl, A substituted with C₃₋₇ cycloalkyl, or Cit or variant thereof;
X5 is absent, a hydrophilic amino acid, or an amino acid with a functional side chain (e.g., Dab, Dap, R, E), wherein the hydrophilic amino acid comprises an L-amino acid comprising —NH₂, —C(O)OH, —NHC(NH)NH₂, —NHC(O)NH₂, —C(O)NH₂, or —NHC(O)CH₃;
X6 is absent, a hydrophilic amino acid, F or a variant thereof, Y or a variant thereof, W or a variant thereof, or N-methylated amino acid thereof, wherein the hydrophilic amino acid comprises a substituent selected from the group consisting of —C(O)OH, —C(O)NH₂, and —NHC(O)CH₃;
X7 is F or a variant thereof, or W or a variant thereof;
X8 is G substituted with one or two straight or branched C₁₋₅ alkyl, A substituted with C₃₋₇ cycloalkyl, or a hydrophilic amino acid wherein the hydrophilic amino acid comprises an L-amino acid comprising —NH₂, one or more —OH, —C(O)OH, —NHC(NH)NH₂, —NHC(O)NH₂, —C(O)NH₂, —NHC(O)CH₃; or the hydrophilic amino acid comprises a zwitterion;
X9 is F or a variant thereof, or W or a variant thereof;
X10 is absent, Q, Hgn, S or variant thereof, T or variant thereof (e.g., T optionally substituted with straight or branched C₁₋₅ alkyl), K or a variant thereof, Cit or a variant thereof, or an L-amino acid substituted with —NHC(NH)NH₂, —NHC(O)NH₂, —C(O)NH₂, or —NHC(O)CH₃;
X11 is absent, E, Hgn, R or a variant thereof, Cit or a variant thereof, Hgl, K or a variant thereof, D, N, or Q; and
X12 is C or a variant thereof.

In some embodiments, described herein is a cyclic peptide that has avidity for ephrin type-A receptor 2 (EphA2), wherein the peptide consists of a sequence of Formula (I),

X1-X2-X3-X4-X5-X6-X7-X8-X9-X10-X11-X12 Formula (I)
or a pharmaceutically acceptable salt thereof,
wherein
   each of X1, X2, X3, X4, X5, X6, and X8 is independently an amino acid;
   X7 is W1Me or a variant thereof;
   X9 is W1Me or a variant thereof;
   each of X10 and X11 is independently absent or an amino acid; and
   X12 is cysteine (C) or a variant thereof.

In some embodiments of a peptide of Formula (I), or a pharmaceutically acceptable salt thereof, wherein:
X7 is W1Me, W1MeCl, W1MeBr, Nal1, Nal2, W1Et, 3Bzf, 3Bzt, F23dC, W1Me7N, or F23dMe;
X8 is V, KCOpipzaa, Hse, N, Cit, hCit, KAc, DapAc, OrnAc, T, alT, Aib, Alb, Qglucamine, Hgl, E, Hgn, MeF, 3Py6NH2, W1Me, A, Q, or K; and
X9 is W1Me, Nal1, W1Et, Nal21N, 3Bzf, 3Bzt, Nal18N, F23dMe, or F23dC.

In some embodiments of a peptide of Formula (I), or a pharmaceutically acceptable salt thereof, wherein:
X7 is W1Me;
X8 is V; and
X9 is W1Me.

In some embodiments of a peptide of Formula (I), or a pharmaceutically acceptable salt thereof, wherein:
X1 is da, df3CON, dkCOpipzaa, dahp, dDab-NH₂-Ph3-SO₂F, dDap-NH₂-Ph3-SO₂F, dDap-NH₂-Ph4-SO₂F, dCit, Aib, G, Norvaline, Norleucine, or dhAla;
X2 is MeF, Me3Py, MeF3CON, MeF3F, Me4Py, MeY (Me), or N-methylated amino acid thereof;
X3 is absent, N, Q, Cit, G, Aib, Hgn, hCit, norCit, LysAc, OrnAc, Ala, or da;
X4 is L, Cbg, Chg, Cba, Cha, Ahx, Dahp, Cit, I, V, Norleucine, or Norvaline;
X5 is Hgl, Hgn, Dab, Dap, DabAc, DapAc, R, hArg, E, or D;
X6 is absent, MeF, MeE, Me3Py, Me4Py, MeF4F, MeF4F, MeF4C, or MeY;
X7 is W1Me, W1Me7Cl, W1Me7N, W, F, 7-AzaTrp, W7Me, or W1Et;
X8 is V, KCOpipzaa, Cit, Qglucamine, hCit, Aib, Norleucine, or Norvaline;
X9 is W1Me, W1Me7Cl, W1Me7N, F23dMe, W1Et, W7Me, W, F, or 7-AzaTrp;
X10 is absent, T, Q, S, Hgn, Alpha-methylserine, hSer, hThr, N, OrnAc, LysAc, Cit, or hCit;
X11 is absent, E, Hgn, R, hArg, Cit, hCit, Hgl, Orn, D, N, Q, DapAc, OrnAc, DabAc, norCit; and
X12 is C, hCys, CdMe, C3RMe, C3SMe, Selenocysteine, dc, or Penicillamine.

In some embodiments, of a peptide of Formula (I), or a pharmaceutically acceptable salt thereof,
X7 is W1Me; and
X9 is W1Me

In some embodiments, the peptide of Formula (I), or a pharmaceutically acceptable salt thereof,

X1-X2-X3-X4-X5-X6-X7-X8-X9-X10-X11-X12 Formula (I)
wherein,
   X1 is any amino acid;
   X2 is an amino acid comprising an aromatic ring or a variant thereof, or N-methylated amino acid thereof;
   X3 is absent, N or a variant thereof;
   X4 is any hydrophobic amino acid or a variant thereof;
   X5 is a hydrophilic amino acid or a variant thereof;
   X6 is absent, a hydrophilic amino acid or amino acid having aromatic ring, or N-methylated amino acid thereof;
   X7 is W or a variant thereof;
   X8 is V, hydrophilic amino acid or a variant thereof, or an N-methylated amino acid;
   X9 is W or a variant thereof;
   X10 is absent, T or a variant thereof;
   X11 is absent, any hydrophilic amino acid; and
   X12 is C or a variant thereof.

In one aspect, described herein is a radiopharmaceutical conjugate comprising:
(a) a cyclic peptide that has avidity for ephrin type-A receptor 2 (EphA2), wherein the peptide has an amino acid sequence of Formula (I),

X1-X2-X3-X4-X5-X6-X7-X8-X9-X10-X11-X12 Formula (I)
wherein,
X1 is any D- or L-amino acid;
X2 has a structure of

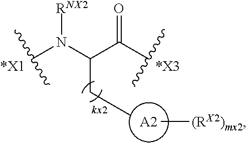

wherein
   ring A2 is phenyl or a 6-membered heteroaryl (e.g., heteroaryl having 1 or 2 N);
   R^{X2} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
   kx2 is 0, 1, 2, or 3;
   mx2 is 0, 1, 2, 3 or 4;
   R^{NX2} is H, C₁-C₆alkyl, or C₁-C₆haloalkyl;
   *X1 indicates the point of attachment to X1; and,
   *X3 indicates the point of attachment to X3;
X3 has a structure of

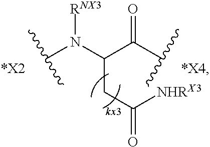

wherein
   kx3 is 0, 1, 2, or 3;
   R^{NX3} is H, C₁-C₆alkyl, or C₁-C₆haloalkyl;
   R^{X3} is H, C₁-C₆alkyl, C₁-C₆haloalkyl, C₁-C₆hydroxyalkyl, C₁-C₆aminoalkyl, or C₁-C₆heteroalkyl;
   *X2 indicates the point of attachment to X2; and,
   *X4 indicates the point of attachment to X4;
X4 is a hydrophobic amino acid (e.g., amino acid having 4 or more carbon atoms in a side chain comprising a linear, branched, or cyclic carbon chain), and wherein X4 is optionally N-alkylated by a C₁₋₃ alkyl group;
X5 is a hydrophilic L-amino acid, such as an amino acid having a structure of

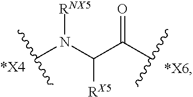

wherein:
   R^{NX5} is H, —CN, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA},
   R^{X5} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═NR^{b})NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA};
   provided that at least one of R^{NX5} and R^{X5} comprises a moiety selected from —OH, —NH₂, and —NH— (e.g., —NH—C(═NH)—NH₂, —CO—NH₂, —NH₂, —COOH, —C(OH)—C₀₋₆ alkyl, —NH—CO—C₁₋₆ alkyl);
   *X4 indicates the point of attachment to X4; and,
   *X6 indicates the point of attachment to X6;
X6 is

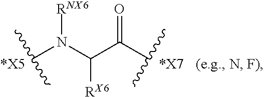

wherein
   R^{NX6} is H, C₁-C₆alkyl, or C₁-C₆haloalkyl;
   R^{X6} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═NR^{b})NR^{c}R^{d}, —NR^{b}C(═O)R^{a}, —NRbC(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl or heteroaryl; wherein the alkyl, haloalkyl, hydroxyalkyl, aminoalkyl, heteroalkyl, cycloalkyl, heterocycloalkyl, aryl or heteroaryl is optionally and independently substituted with one or more R^{XA};
   *X5 indicates the point of attachment to X5; and,
   *X7 indicates the point of attachment to X7;

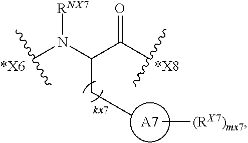

X7 has a structure of wherein
   R^{NX7} is H, C₁-C₆alkyl, or C₁-C₆haloalkyl;
   ring A7 is an aryl or heteroaryl;
   R^{X7} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂-halogen, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-Coalkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
   kx7 is 0, 1, 2, or 3;
   mx7 is 0, 1, 2, 3, 4 or 5;
   *X6 indicates the point of attachment to X6; and,
   *X8 indicates the point of attachment to X8;
X8 is an L-amino acid with —H on the alpha-amino group;

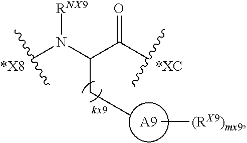

X9 has a structure of wherein
   R^{NX9} is H, C₁-C₆alkyl, or C₁-C₆haloalkyl;
   ring A9 is an aryl or heteroaryl;
   R^{X9} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
   kx9 is 0, 1, 2, or 3;
   mx9 is 0, 1, 2, 3, 4, or 5;
   *X8 indicates the point of attachment to X8; and,
   *XC indicates the point of attachment to (i) X10 or (i) when X10 and X11 are absent,
X12;
X10 is absent or an L-amino acid;
X11 is absent or an L-amino acid; provided that when X10 is absent, then X11 is also absent; and
X12 is an L-amino acid having a reactive thiol group, such as Cys and Cys variants;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R and R^{XA} is independently halogen, —CN, —OH, —OC₁-C₆alkyl, SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NR^{b}C(═NR^{b})NR^{c}R^{d}, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
(b)(i) a metal chelator configured to bind with a radionuclide; or (ii) a covalent radionuclide; and
(c)(i) optionally, a linker that connects the peptide with the metal chelator; or (ii) optionally a linker that connects the peptide with the covalent radionuclide.

In some embodiments, X3 has a structure of

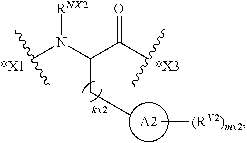

wherein the definitions for the groups are provided herein. In some embodiments, A2 is phenyl. In some embodiments, A2 is 6-membered heteroaryl. In some embodiments, R^{X2} is each independently halogen, —CN, —NO₂, —OH, —OR^{a}, —OC(═O)R^{a}, —SH, —S(═O)₂NR^{c}R^{d}, —NR^{c}R^{d}, —NR^{b}C(═O)R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA}. In some embodiments, kx2 is 0. In some embodiments, kx2 is 1. In some embodiments, kx2 is 2. In some embodiments, kx2 is 3. In some embodiments, mx2 is 0. In some embodiments, mx2 is 1. In some embodiments, mx2 is 2. In some embodiments, mx2 is 3. In some embodiments, mx2 is 4. In some embodiments, R^{NX2} is H. In some embodiments, R^{NX2} is methyl.

In some embodiments, X3 has a structure of

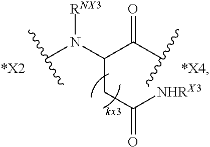

wherein the definitions for the groups are provided herein. In some embodiments, kx3 is 0. In some embodiments, kx3 is 1. In some embodiments, kx3 is 2. In some embodiments, kx3 is 3. In some embodiments, R^{NX3} is H. In some embodiments, R^{NX3} is methyl. In some embodiments, R^{X3} is H. In some embodiments, R^{X3} is C₁-C₆alkyl. In some embodiments, R^{X3} is C₁-C₃alkyl.

In some embodiments, X5 has a structure of

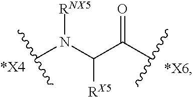

wherein the definitions for the groups are provided herein. In some embodiments, R^{NX5} is H. In some embodiments, R^{NX5} is methyl. In some embodiments, R^{X5} is —CN, —NO₂, —OH, —OR^{a}, —OC(═O)R^{a}, —SH, —NR^{c}R^{d}, —NR^{b}C(═O)R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA}. In some embodiments, at least one of R^{NX5} and R^{X5} comprises a moiety selected from —NH—C(═NH)—NH₂, —CO—NH₂, —NH₂, —COOH, —C(OH)—C₀₋₆ alkyl, —NH—CO—C₁₋₆ alkyl. In some embodiments, at least one of R^{NX5} and R^{X5} comprises a moiety selected from —CO—NH₂.

In some embodiments, X6 has a structure of

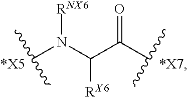

wherein the definitions for the groups are provided herein. In some embodiments, R^{NX6} is H. In some embodiments, R^{NX6} is methyl. In some embodiments, R^{X6} is C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA}. In some embodiments, R^{X6} is C₁-C₆alkyl, which is optionally substituted.

In some embodiments, X7 has a structure of

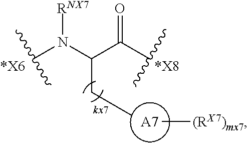

wherein the definitions for the groups are provided herein. In some embodiments, ring A7 is a 6-membered aryl or heteroaryl. In some embodiments, ring A7 is a 9- or 10-membered bicyclic aryl or heteroaryl. In some embodiments, ring A7 is bicyclic heteroaryl, which is optionally substituted. In some embodiments, the 6-, 9- or 10-membered heteroaryl has one heteroatom selected from N, O, and S. In some embodiments, ring A7 is optionally substituted 5-6, 6-6, or 6-5 fused heteroaryl. In some embodiments, ring A7 is optionally substituted 5-6 or 6-5 fused heteroaryl. In some embodiments, R^{NX7} is H. In some embodiments, each of R^{X7} is independently halogen, —CN, —NO₂, —OH, —OR^{a}, amino, C₁-C₆alkyl, or C₁-C₆haloalkyl. In some embodiments, R^{X7} is each independently halogen, —CN, —NO₂, —OH, —OR^{a}, —OC(═O)R^{a}, —SH, —NR^{c}R^{d}, —NR^{b}C(═O)R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA}. In some embodiments, each R^{X7} is independently selected from —CH₃, -ethyl, —Cl, and —F, and mx7 is 0, 1, or 2. In some embodiments, mx7 is 0. In some embodiments, mx7 is 1. In some embodiments, mx7 is 2. In some embodiments, mx7 is 3-4. In some embodiments, kx7 is 0. In some embodiments, kx7 is 1. In some embodiments, kx7 is 2. In some embodiments, kx7 is 3.

In some embodiments, X7 is W1Me, Nal1, Nal2, W1Et, Nal21N, 3Bzf, 3Bzt, Nal15N, Nal14N, Nal24N, Nal28N, F23dMe, F23dC, W1Me7N, or W1Me7Cl. In some embodiments, X7 is W1Me, F23dMe or W1Me7Cl.

In some embodiments, X9 has a structure of

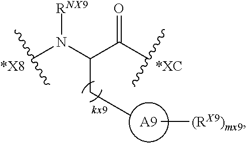

wherein the definitions for the groups are provided herein. In some embodiments, X9 is

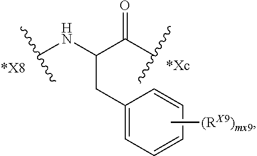

wherein each R^{X9} is independently selected from —OH, CN, NH₂, C₁-C₃alkyl, —C1, —F, —Br, —CONH₂, and —SO₂F.

In some embodiments, ring A9 is bicyclic heteroaryl, which is optionally substituted. In some embodiments, ring A9 is optionally substituted 5-6, 6-6, or 6-5 fused heteroaryl. In some embodiments, ring A9 is optionally substituted 5-6 or 6-5 fused heteroaryl. In some embodiments,

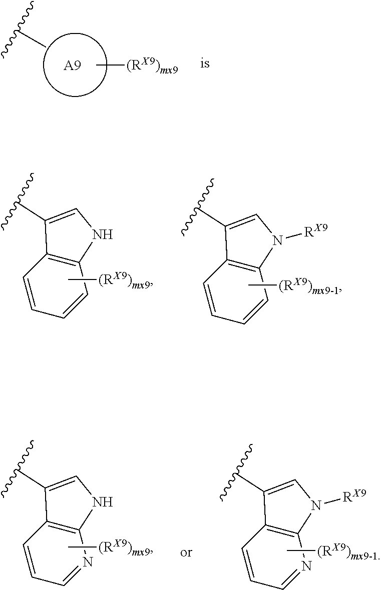

In some embodiments,

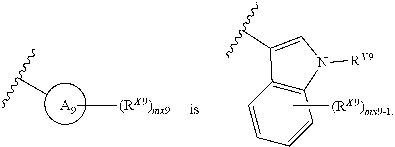

In some embodiments, mx9 is 0. In some embodiments, mx9 is 1. In some embodiments, mx9 is 2.

In some embodiments, each of R^{X9} is independently halogen, —CN, —NO₂, —OH, —OR^{a}, —OC(═O)R^{a}, —SH, —SR^{a}, —S(═O)R^{a}, —S(═O)₂R^{a}, —S(═O)₂NR^{c}R^{d}, —NR^{c}R^{d}, —NR^{b}C(═O)R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl. In some embodiments, each of R^{X9} is independently halogen, —CN, —NO₂, —OH, —OR^{a}, amino, C₁-C₆alkyl, or C₁-C₆haloalkyl. In some embodiments, R^{NX9} is H. In some embodiments, R^{NX9} is methyl. In some embodiments, kx9 is 0. In some embodiments, kx9 is 1. In some embodiments, kx9 is 2. In some embodiments, kx9 is 3. In some embodiments, mx9 is 0. In some embodiments, mx9 is 1. In some embodiments, mx9 is 2. In some embodiments, mx9 is 3.

In some embodiments, X9 is W1Me, W, Nal1, W1Et, Nal21N, 3Bzf, 3Bzt, Nal14N, Nal18N, F23dMe, F23dC, or W1Et. In some embodiments, X9 is W1Me or F23dMe.

In some embodiments, ring A2 is a 6-membered heteroaryl containing 1 or 2 N.

In some embodiments, R^{X5} is C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl.

In some embodiments of Formulas (I), (I-1), (I-2), (I-3), (I-4), (I-5), (Ia), (Ib), (Ic), (III-1), (III-2), (III-1-RI), and (III-2-RI), X1 is any amino acid (e.g., D-amino acid). In some embodiments, X1 is any one of the canonical amino acids. In some embodiments, X1 is an unnatural amino acid. In some embodiments, X1 is N-alkylated amino acid. In some embodiments, X1 is alanine (A). In some embodiments, X1 is D-alanine. In some embodiments, X1 is df3CON. In some embodiments, X1 is dkCOpipzaa. In some embodiments, X1 is dahp. In some embodiments, X1 is F. In some embodiments, X1 is an amino acid selected from Tables 5A to 5F. In some embodiments, the metal chelator or linker is attached to X1. In some embodiments of Formulas (I), (I-1), (I-2), (I-3), (I-4), (I-5), (Ia), (Ib), (Ic), (III-1), (III-2), (III-1-RI), and (III-2-RI), X1 is any amino acid. In some embodiments, X1 is an amino acid (e.g., a D-amino acid). In some embodiments, X1 is da, df3CON, dkCOpipzaa, dahp, dDab-NH₂-Ph3-SO₂F, dDap-NH₂-Ph3-SO₂F, dDap-NH₂-Ph4-SO₂F, dCit, Aib, G, Norvaline, Norleucine, or dhAla. X1 is da. X1 is df3CON. X1 is dkCOpipzaa. X1 is dahp. X1 is dDab-NH₂-Ph3-SO₂F. X1 is dDap-NH₂-Ph3-SO₂F. X1 is dCit. X1 is Aib. X1 is G. X1 is Norvaline. X1 is Norleucine. X1 is dhAla. In some embodiments, X1 is F. In some embodiments, X1 is chloroacetylated. In some embodiments, X1 is bromoacetylated. In some embodiments, X1 comprises a chloroacetyl group. In some embodiments, X1 comprises a bromoacetyl group. In some embodiments, in the cyclic peptide, the chloroacetyl or bromoacetyl group has been reacted and is no longer present in X1.

In some embodiments of Formulas (I), (I-1), (I-2), (I-3), (I-4), (I-5), (Ia), (Ib), (Ic), (III-1), (III-2), (III-1-RI), and (III-2-RI), X2 is a canonical amino acid. In some embodiments, X2 is an unnatural amino acid. In some embodiments, X2 is an aromatic amino acid or a variant thereof. In some embodiments, X2 is V. In some embodiments, X2 is an N-methylated amino acid or a variant thereof. In some embodiments, X2 is an N-alkylated amino acid or a variant thereof. In some embodiments, X2 is an amino acid comprising an aryl group. In some embodiments, X2 is an amino acid comprising an optionally substituted phenyl group. In some embodiments, X2 is an amino acid comprising an optionally substituted naphthyl group. In some embodiments, X2 is an amino acid comprising a heteroaryl group. In some embodiments, X2 is an amino acid comprising an optionally substituted monocyclic heteroaryl group. In some embodiments, X2 is an amino acid comprising an optionally substituted bicyclic heteroaryl group. In some embodiments, the aryl or heteroaryl is optionally substituted with 1, 2, or 3 substituents independently selected from —CH₃, -ethyl, —Cl, and —F. In some embodiments, the aryl or heteroaryl is optionally substituted with 1, 2, or 3 substituents independently selected from —OH, oxo, halogen, CN, amino, C₁-C₆ alkyl, C₁-C₆ alkoxyl, and C₁-C₆ haloalkyl. In some embodiments, X2 is F, or a variant thereof that replaces the unsubstituted phenyl ring of F with (i) a phenyl ring substituted by 1 or 2 substituents each independently selected from —OH, —CN, —C₁₋₃ alkyl, or (ii) a 6-membered heteroaryl ring optionally substituted by 1 or 2 substituents each independently selected from —OH, —CN, —C₁₋₃ alkyl, wherein the F or the variant thereof is optionally N-methylated. In some embodiments, X2 is Me3Py. In some embodiments, X2 is In some embodiments, X2 is MeF. In some embodiments, X2 is MeF3H. In some embodiments, X2 is MeF3CN. In some embodiments, X2 is MeF3H. In some embodiments, X2 is Me4Py2NH₂. In some embodiments, X2 is 4Py2NH2. In some embodiments, X2 is 4Py. In some embodiments, X2 is Me3Py. In some embodiments, X2 is an amino acid substituted with an aryl or heteroaryl. In some embodiments, X2 is histidine (H). In some embodiments, X2 is phenylalanine, tryptophan, tyrosine, or a variant thereof. In some embodiments, X2 is phenylalanine or a variant thereof. In some embodiments, X2 is tryptophan or a variant thereof. In some embodiments, X2 is W1Me. In some embodiments, X2 is tyrosine or a variant thereof. In some embodiments, X2 is absent. In some embodiments, the metal chelator or linker is attached to X2. In some embodiments of Formulas (I), (I-1), (I-2), (I-3), (I-4), (I-5), (III-1), (Ia), (Ib), (Ic), (III-2), (III-1-RI), and (III-2-RI), X2 is an amino acid comprising an aromatic ring, or N-methylated amino acid thereof. In some embodiments, X2 is N-methylated amino acid. In some embodiments, X2 is an amino acid comprising an aromatic ring. In some embodiments, X2 is an N-methylated amino acid comprising an aromatic ring. In some embodiments, X2 is F or a variant thereof, Y or a variant thereof, or W or a variant thereof, or N-methylated amino acid thereof. In some embodiments, X2 is F or a variant thereof. In some embodiments, X2 is N-methyl F or a variant thereof. In some embodiments, X2 is Y or a variant thereof. In some embodiments, X2 is N-methyl Y or a variant thereof. In some embodiments, X2 is W or a variant thereof. In some embodiments, X2 is N-methyl W or a variant thereof. In some embodiments, X2 is MeF, Me3Py, MeF3CON, MeF3F, Me4Py, or MeY (Me). In some embodiments, X2 is MeF. In some embodiments, X2 is Me3Py. In some embodiments, X2 is MeF3CON. In some embodiments, X2 is MeF3F. In some embodiments, X2 is Me4Py. In some embodiments, X2 is MeY. In some embodiments, X2 is MeY (Me).

In some embodiments of Formulas (I), (I-1), (I-2), (I-3), (I-4), (I-5), (Ia), (III-1), (III-2), (III-1-RI), and (III-2-RI), X3 is a canonical amino acid. In some embodiments, X3 is an unnatural amino acid. In some embodiments, X3 is N-alkylated amino acid. In some embodiments, X3 is asparagine (N). In some embodiments, X3 is a substitute of asparagine. In some embodiments, X3 is absent. In some embodiments of Formulas (I), (I-1), (I-2), (I-3), (I-4), (I-5), (III-1), (Ia), (III-2), (III-1-RI), and (III-2-RI), X3 is absent. In some embodiments of Formulas (I), (I-1), (I-2), (I-3), (I-4), (I-5), (III-1), (Ia), (III-2), (III-1-RI), and (III-2-RI), X3 is a hydrophilic amino acid (e.g. N, Hgn, Q, Cit, K or a variant thereof), glycine (G), Alanine (A) or a variant thereof (e.g., da, 2-Aminoisobutyric acid (Aib). In some embodiments, X3 is a hydrophilic amino acid. In some embodiments, X3 is an amino acid comprising-OH, —NH₂, —C(O)OH, —NHC(═NH)NH₂, —NHC(O)NH₂, —C(O)NH₂, or —NHC(O)CH₃ group. In some embodiments, X3 has an electrically charged side chain. In some embodiments, X3 has a positively charged side chain. In some embodiments, X3 has a negatively charged side chain. In some embodiments of Formulas (I), (I-1), (I-2), (I-3), (I-4), (I-5), (III-1), (Ia), (III-2), (III-1-RI), and (III-2-RI), X3 is an amino acid comprising an electrically charged side chain (e.g., K or a variant thereof), an amino acid comprising a polar uncharged side chain (e.g., Q, Cit, N, or a variant thereof), or G, A or variant thereof. In some embodiments, X3 is an amino acid comprising an electrically charged side chain. In some embodiments, X3 is an amino acid comprising a polar uncharged side chain. In some embodiments, X3 has zwitterionic (e.g., KCOpipzaa) side chain. In some embodiments, X3 is zwitterionic. In some embodiments, X3 comprises a —OH, —COOH, —NH— or NH₂ moiety. In some embodiments, X3 comprises —OH, —C(O)OH, —NHC(═NH)NH₂, —NHC(O)NH₂, —C(O)NH₂, or —NHC(O)CH₃. In some embodiments, X3 comprises a side chain of C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl. In some embodiments, X3 is absent, a hydrophilic amino acid (e.g. N, Q, Hgn, Cit, K or a variant thereof), G, Ala, or a variant thereof (e.g., da, Aib,). In some embodiments, X3 is N, Q, K, G, S, T, E, Aib, Hcit, Cit, Hgn, KCOpipzaa, Har, Nmm, Ndm, Ala, Hgl, 3Py6NH2, or a variant thereof including D-amino acid such as da and variations such as Qglucamine. In some embodiments X3 is absent, N, Q, Cit or a variant thereof, G, Aib, Hgn, K or a variant thereof, or Ala or a variant thereof (e.g., da). In some embodiments, X3 is absent, N, Q, Cit, G, Aib, Hgn, hCit, norCit, LysAc, OrnAc, Ala, or da. In some embodiments, X3 is N or a variant thereof. In some embodiments, X3 is N. In some embodiments, X3 is Q or a variant thereof. In some embodiments, X3 is Q. In some embodiments, X3 is Cit or a variant thereof. In some embodiments, X3 is Cit, hCit, or norCit. In some embodiments, X3 is Cit. in some embodiments, X3 is hCit. In some embodiments, X3 is norCit. In some embodiments, X3 is K or a substitution there of. In some embodiments, X3 is K, LysAc, or OrnAc. In some embodiments. X3 is K. In some embodiments, X3 is LysAc. In some embodiments, X3 is OrnAc. In some embodiments, X3 is G or a variant thereof. In some embodiments, X3 is G. In some embodiments, X3 is Hgn. In some embodiments, X3 is Aib. In some embodiments, X3 is Ala or a variant thereof. In some embodiments, X3 is Ala or da. In some embodiments, X3 is Ala. In some embodiments, X3 is da. In some embodiments, X3 is absent. In some embodiments, the metal chelator or linker is attached to X3. In some embodiments, the covalently bound radionuclide or linker is attached to X3. In some embodiments, X1 is directly bound to X3.

In some embodiments of Formulas (I), (I-1), (I-2), (I-3), (I-4), (I-5), (Ia), (Ib), (III-1), (III-2), (III-1-RI), and (III-2-RI), X4 is a hydrophobic amino acid or a variant thereof. In some embodiments, X4 is an unnatural amino acid. In some embodiments, X4 is a canonical amino acid. In some embodiments, X4 is leucine. In some embodiments, X4 comprises 4 or more carbon atoms in a side chain comprising a linear, branched, or cyclic carbon chain. In some embodiments, X4 comprises 4 or more contiguous carbon atoms in a side chain. In some embodiments, X4 comprises an ethylene, propylene, or butylene group in a side chain. In some embodiments, X4 is Cbg. In some embodiments, X4 is absent. In some embodiments, X4 is selected from glycine (G), methionine (M), alanine (A), valine (V), leucine (L), isoleucine (I), proline (P), phenylalanine (F), cysteine (C), substitutes thereof. In some embodiments of Formulas (I), (I-1), (I-2), (I-3), (I-4), (I-5), (Ia), (Ib), (III-1), (III-2), (III-1-RI), and (III-2-RI), X4 is an amino acid comprising a hydrophobic side chain (e.g., L), an amino acid comprising a polar uncharged side chain (e.g., Cit or a variant thereof). In some embodiments, X4 is an amino acid comprising a hydrophobic side chain. In some embodiments, X4 is an amino acid comprising a polar uncharged side chain. In some embodiments of Formulas (I), (I-1), (I-2), (I-3), (I-4), (I-5), (Ia), (Ib), (III-1), (III-2), (III-1-RI), and (III-2-RI), X4 is absent, a hydrophobic amino acid, or a hydrophilic amino acid (e.g., Cit or a variant thereof). In some embodiments, X4 is absent, G substituted with straight or branched C₁₋₅ alkyl, A substituted with C₃₋₇ cycloalkyl, or Cit or variant thereof. In some embodiments, X4 is absent, L, Cbg, Chg, Cba, Cha, Ahx, Dahp, citrulline (Cit), I, V, Norleucine, or Norvaline. In some embodiments, X4 is absent. In some embodiments, X4 is a hydrophobic amino acid. In some embodiments, X4 is Leu, Hcit, Cbg, Chg, or Cba. In some embodiments, X4 is Leu, Cbg, Chg or Cba. In some embodiments, X4 is G substituted with straight or branched C₁₋₅ alkyl. In some embodiments, X4 is G substituted with methyl, ethyl, propyl, isopropyl, butyl, isobutyl, pentyl, or isopentyl. In some embodiments, X4 A substituted with C₃₋₇ cycloalkyl. In some embodiments, X4 is A substituted with cyclopropyl. In some embodiments, X4 is A substituted with cyclobutyl. In some embodiments, X4 is A substituted with cyclopentyl. In some embodiments, X4 is A substituted with cyclohexyl. In some embodiments, X4 is A substituted with cycloheptyl. In some embodiments, X4 is L, Cbg, Chg, Cba, Cha, Ahx, Dahp, I, V, Norleucine, or Norvaline. In some embodiments, X4 is L. In some embodiments, X4 is Cbg. In some embodiments, X4 is Chg. In some embodiments, X4 is Cba. In some embodiments, X4 is Cha. In some embodiments, X4 is Ahx. In some embodiments, X4 is Dahp. In some embodiments, X4 is I. In some embodiments, X4 is V. In some embodiments, X4 is Norleucine. In some embodiments, X4 is Norvaline. In some embodiments, X4 is a hydrophilic amino acid. In some embodiments, X4 is Cit or a variant thereof. In some embodiments, X4 is Cit. In some embodiments, X4 is optionally N-methylated. In some embodiments, the metal chelator or linker is attached to X4. In some embodiments, X1 is directly bound to X4. In some embodiments of Formulas (I), (I-1), (I-2), (I-3), (I-4), (I-5), (Ia), (Ib), (III-1), (III-2), (III-1-RI), and (III-2-RI), X4 is a hydrophilic amino acid. In some embodiments, X4 is an amino acid comprising —OH, —NH₂, —C(O)OH, —NHC(═NH)NH₂, —NHC(O)NH₂, —C(O)NH₂, or —NHC(O)CH₃ group. In some embodiments, X4 has an electrically charged side chain. In some embodiments, X4 has a positively charged side chain. In some embodiments, X4 has a negatively charged side chain. In some embodiments, X4 is zwitterionic. In some embodiments, X4 comprises a —OH, —COOH, —NH— or NH₂ moiety. In some embodiments, X4 comprises —OH, —C(O)OH, —NHC(═NH)NH₂, —NHC(O)NH₂, —C(O)NH₂, or —NHC(O)CH₃. In some embodiments, X4 comprises a side chain of C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl. In some embodiments of Formulas (I), (I-1), (I-2), (I-3), (I-4), (I-5), (Ia), (Ib), (III-1), (III-2), (III-1-RI), and (III-2-RI), X4 is a hydrophobic amino acid. In some embodiments, X4 comprises at least 4 contiguous carbon atoms, either linear or branched. In some embodiments, X4 comprises at least 5 contiguous carbon atoms, either linear or branched. In some embodiments, X4 comprises a propylene moiety in the side chain. In some embodiments, X4 comprises a butylene moiety in the side chain.

In some embodiments of Formulas (I), (I-1), (I-2), (I-3), (I-4), (I-5), (Ia), (Ib), (III-1), (III-2), (III-1-RI), and (III-2-RI), X5 is a hydrophilic amino acid or a variant thereof. In some embodiments, X5 is a hydrophilic amino acid. In some embodiments, X5 is an unnatural amino acid. In some embodiments, X5 is a positively charged amino acid. In some embodiments, X5 is a negatively charged amino acid. In some embodiments, X5 is not charged. In some embodiments, X5 is a canonical amino acid. In some embodiments, X5 is N-alkylated amino acid. In some embodiments, X5 is Ala or a variant thereof. In some embodiments, X5 is N, Q, K, G, S, T, E, Aib, Hcit, Cit, Hgn, KCOpipzaa, Har, Nmm, Ndm, Ala, Hgl, 3Py6NH2, or a variant thereof including D-amino acid such as da and variations such as Qglucamine. In some embodiments, X5 is Hgn, N, Qglucamine, KCOpipzaa, Hgl, Nmm, Ndm, KCOpipzaa, K, S, T, or E. In some embodiments, X5 is Hgn. In some embodiments, X5 is asparagine (N). In some embodiments, X5 is Qglucamine. In some embodiments, X5 is Hgl. In some embodiments, X5 is Nmm. In some embodiments, X5 is Ndm. In some embodiments, X5 is KCOpipzaa. In some embodiments, X5 is Dab. In some embodiments, X5 is S. In some embodiments, X5 is K. In some embodiments, X5 is absent. In some embodiments of Formulas (I), (I-1), (I-2), (I-3), (I-4), (I-5), (Ia), (Ib), (III-1), (III-2), (III-1-RI), and (III-2-RI), X5 is an amino acid comprising an electrically charged side chain (e.g., E, Hgl, D, or a variant thereof), or an amino acid comprising a polar uncharged side chain (e.g., Q, Cit, Hgn, N, or a variant thereof). In some embodiments, X5 is an amino acid comprising an electrically charged side chain. In some embodiments, X5 is an amino acid comprising a polar uncharged side chain. In some embodiments of Formulas (I), (I-1), (I-2), (I-3), (I-4), (I-5), (III-1), (Ia), (Ib), (III-2), (III-1-RI), and (III-2-RI), X5 is absent, a hydrophilic amino acid, or a variant thereof. In some embodiments, X5 is absent, a hydrophilic amino acid, or an amino acid with a functional side chain (e.g., Dab, Dap, R, E), wherein the hydrophilic amino acid comprises an L-amino acid comprising —NH₂, —C(O)OH, —NHC(NH)NH₂, —NHC(O)NH₂, —C(O)NH₂, or —NHC(O)CH₃. In some embodiments, X5 is absent, Hgl, Hgn, Dab, Dap, DabAc, DapAc, R, hArg, E, or D. In some embodiments, X5 is absent. In some embodiments, X5 is a hydrophilic amino acid. In some embodiments, X5 is an amino acid comprising —NH₂, —C(O)OH, —NHC(NH)NH₂, —NHC(O)NH₂, —C(O)NH₂, or —NHC(O)CH₃. In some embodiments, X5 is an L-amino acid comprising —NH₂, —C(O)OH, —NHC(NH)NH₂, —NHC(O)NH₂, —C(O)NH₂, or —NHC(O)CH₃. In some embodiments, X5 is Hgl. In some embodiments, X5 is Hgn. In some embodiments, X5 is Dab. In some embodiments, X5 is Dap. In some embodiments, X5 is DabAc. In some embodiments, X5 is DapAc. In some embodiments, X5 is R or a variant thereof. In some embodiments, X5 is R or hArg. In some embodiments, X5 is R. In some embodiments, X5 is hArg. In some embodiments, X5 is E. In some embodiments, X5 is hCit. In some embodiments, X5 is G. In some embodiments, X5 is D. In some embodiments, the metal chelator or linker is attached to X5. In some embodiments, the covalently bound radionuclide or linker is attached to X5. In some embodiments, X1 is directly bound to X5.

In some embodiments of Formulas (I), (I-1), (I-2), (I-3), (I-4), (I-5), (Ia), (Ic), (III-1), (III-2), (III-1-RI), and (III-2-RI), X6 is any amino acid. In some embodiments, X6 is a canonical amino acid. In some embodiments, X6 is an unnatural amino acid. In some embodiments, X6 is hydrophilic amino acid or amino acid having aromatic ring, or N-methylated amino acid thereof, or a substitute thereof. In some embodiments, X6 is an amino acid having aromatic ring or a substitute thereof. In some embodiments, X6 is an amino acid comprising an aryl group. In some embodiments, X6 is an amino acid comprising an optionally substituted phenyl group. In some embodiments, X6 is an amino acid comprising an optionally substituted naphthyl group. In some embodiments, X6 is an amino acid comprising a heteroaryl group. In some embodiments, X6 is an amino acid comprising an optionally substituted monocyclic heteroaryl group. In some embodiments, X6 is an amino acid comprising an optionally substituted bicyclic heteroaryl group. In some embodiments, the aryl or heteroaryl is optionally substituted with 1, 2, or 3 substituents independently selected from —CH₃, -ethyl, —Cl, and —F. In some embodiments, the aryl or heteroaryl is optionally substituted with 1, 2, or 3 substituents independently selected from —OH, oxo, halogen, CN, amino, C₁-C₆ alkyl, C₁-C₆ alkoxyl, and C₁-C₆ haloalkyl. In some embodiments, X6 is N-methylated amino acid. In some embodiments, X6 is hydrophilic amino acid or a substitute thereof. In some embodiments, X6 is an amino acid having aromatic ring or a substitute thereof. In some embodiments, X6 is an N-methylated amino acid or a substitute thereof. In some embodiments, X6 is MeE. In some embodiments, X6 is N. In some embodiments, X6 is MeN. In some embodiments, X6 is Me3Py. In some embodiments, X6 is MeF. In some embodiments, X6 is Qglucamine. In some embodiments, X6 is MeF4C. In some embodiments, X6 is absent. In some embodiments of Formulas (I), (I-1), (I-2), (I-3), (I-4), (I-5), (Ia), (Ic), (III-1), (III-2), (III-1-RI), and (III-2-RI), X6 is an amino acid comprising an electrically charged side chain (e.g., E, Hgl, D, or a variant thereof), or an amino acid comprising a polar uncharged side chain (e.g., Q, Cit, Hgn, N, or variant). In some embodiments, X6 is an amino acid comprising an electrically charged side chain. In some embodiments, X6 is an amino acid comprising a polar uncharged side chain. In some embodiments of Formulas (I), (I-1), (I-2), (I-3), (I-4), (I-5), (III-1), (Ia), (Ic), (III-2), (III-1-RI), and (III-2-RI), X6 is absent, a hydrophilic amino acid, an amino acid comprising an aromatic ring, or N-methylated amino acid thereof. In some embodiments of Formulas (I), (I-1), (I-2), (I-3), (I-4), (I-5), (Ia), (Ib), (III-1), (III-2), (III-1-RI), and (III-2-RI), X6 is a hydrophilic amino acid. In some embodiments, X6 is an amino acid comprising —OH, —NH₂, —C(O)OH, —NHC(═NH)NH₂, —NHC(O)NH₂, —C(O)NH₂, or —NHC(O)CH₃ group. In some embodiments, X6 has an electrically charged side chain. In some embodiments, X6 has a positively charged side chain. In some embodiments, X6 has a negatively charged side chain. In some embodiments, X6 is zwitterionic. In some embodiments, X6 comprises a —OH, —COOH, —NH— or NH₂ moiety. In some embodiments, X6 comprises —OH, —C(O)OH, —NHC(═NH)NH₂, —NHC(O)NH₂, —C(O)NH₂, or —NHC(O)CH₃. In some embodiments, X6 comprises a side chain of C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl. In some embodiments, X6 is absent, a hydrophilic amino acid, F or a variant thereof, Y or a variant thereof, W or a variant thereof, or N-methylated amino acid thereof, wherein the hydrophilic amino acid comprises a substituent selected from the group consisting of —C(O)OH, —C(O)NH₂, and —NHC(O)CH₃. In some embodiments, X6 is absent, MeF, MeE, Me3Py, Me4Py, MeF4F, MeF4C, or MeY. In some embodiments, X6 is MeE, MeN, Me3Py, MeF, MeF4C, or N. In some embodiments, X6 is absent. In some embodiments, X6 is a hydrophilic amino acid. In some embodiments, X6 is an amino acid comprising —NH₂, —C(O)OH, —NHC(NH)NH₂, —NHC(O)NH₂, —C(O)NH₂, or —NHC(O)CH₃. In some embodiments, X6 is E or N-methylated amino acid thereof. In some embodiments, X6 is E. In some embodiments, X6 is MeE. In some embodiments, X6 is an amino acid comprising an aromatic ring or N-methylated amino acid thereof. In some embodiments, X6 is an amino acid comprising an optionally substituted phenyl. In some embodiments, X6 is an amino acid comprising an optionally substituted heteroaryl. In some embodiments, X6 is F or a variant thereof, or N-methylated amino acid thereof. In some embodiments, X6 is F, MeF, Me3Py, Me4Py, MeF4F, or MeF4C. In some embodiments, X6 is F. In some embodiments, X6 is MeF. In some embodiments, X6 is Me3Py. In some embodiments, X6 is Me4Py. In some embodiments, X6 is MeF4F. In some embodiments, X6 is MeF4C. In some embodiments, X6 is Y or a variant thereof, or N-methylated amino acid thereof. In some embodiments, X6 is Y or MeY. In some embodiments, X6 is Y. In some embodiments, X6 is MeY. In some embodiments, the metal chelator or linker is attached to X6. In some embodiments, the covalently bound radionuclide or linker is attached to X6. In some embodiments, X1 is directly bound to X6.

In some embodiments of Formulas (I), (I-1), (I-2), (I-3), (I-4), (I-5), (Ia), (Ib), (Ic), (III-1), (III-2), (III-1-RI), and (III-2-RI), X7 is W or a variant thereof. In some embodiments, X7 is a canonical amino acid. In some embodiments, X7 is an unnatural amino acid. In some embodiments, X7 is N-alkylated amino acid. In some embodiments, X7 is W1Me. In some embodiments, X7 is W1Me7Cl. In some embodiments, X7 is W1Me7N. In some embodiments, X7 is absent. In some embodiments, X7 is an amino acid having aromatic ring or a substitute thereof. In some embodiments, X7 is an amino acid comprising an aryl group. In some embodiments, X7 is an amino acid comprising an optionally substituted phenyl group. In some embodiments, X7 is an amino acid comprising an optionally substituted naphthyl group. In some embodiments, X7 is an amino acid comprising a heteroaryl group. In some embodiments, X7 is an amino acid comprising an optionally substituted monocyclic heteroaryl group. In some embodiments, X7 is an amino acid comprising an optionally substituted bicyclic heteroaryl group. In some embodiments, the aryl or heteroaryl is optionally substituted with 1, 2, or 3 substituents independently selected from —CH₃, -ethyl, —Cl, and —F. In some embodiments, the aryl or heteroaryl is optionally substituted with 1, 2, or 3 substituents independently selected from —OH, oxo, halogen, CN, amino, C₁-C₆ alkyl, C₁-C₆ alkoxyl, and C₁-C₆ haloalkyl. In some embodiments, X7 is W, Y, or a variant thereof (such as an amino acid having either a 6-membered aryl or heteroaryl, or a 9- or 10-membered bi-cyclic aryl or heteroaryl linked to the alpha-carbon through a carbon (e.g., a methylene group), wherein the 6-, 9-, and 10-membered heteroaryl has one heteroatom (e.g., N), and wherein the 6-, 9-, and 10-membered aryl or heteroaryl is optionally substituted by 1 or 2 substituents independently selected from —CH₃, -ethyl, —Cl, and —F). In some embodiments of Formulas (I), (I-1), (I-2), (I-3), (I-4), (I-5), (Ia), (Ib), (Ic), (III-1), (III-2), (III-1-RI), and (III-2-RI), X7 is an amino acid comprising an aromatic ring. In some embodiments, X7 is an amino acid comprising an aromatic ring (e.g., W or a variant thereof). In some embodiments, X7 is F or a variant thereof, or W or a variant thereof. In some embodiments, X7 is W1Me, W1Me7Cl, W1Me7N, W, F, 7-AzaTrp, W7Me, or W1Et. In some embodiments, X7 is F or a variant thereof. In some embodiments, X7 is F. In some embodiments, X7 is W or a variant thereof. In some embodiments, X7 is Nal1, Nal2, W1Et, Nal21N, 3Bzf, 3Bzt, Nal15N, Nal14N, Nal24N, Nal28N, F23dC, W1Me, W1Me7Cl, or W1Me7N. In some embodiments, X7 is W1Me, W1Me7Cl, W1Me7N, W, 7-AzaTrp, W7Me, or W1Et. In some embodiments, X7 is W1Me, W1Me7Cl, or F23dMe. In some embodiments, X7 is W1Me, W1Me7Cl, or W1Me7N. In some embodiments, X7 is W1Me. In some embodiments, X7 is W1Me7Cl. In some embodiments, X7 is W1Me7N. In some embodiments, X7 is W. In some embodiments, X7 is 7-AzaTrp. In some embodiments, X7 is W7Me. In some embodiments, the metal chelator or linker is attached to X7. In some embodiments, the covalently bound radionuclide or linker is attached to X7. In some embodiments, X1 is directly bound to X7.

In some embodiments of Formulas (I), (I-1), (I-2), (I-3), (I-4), (I-5), (Ia), (Ib), (Ic), (III-1), (III-2), (III-1-RI), and (III-2-RI), X8 is any amino acid. In some embodiments, X8 is any one of the canonical amino acids. In some embodiments, X8 is an unnatural amino acid. In some embodiments, X8 is V, hydrophilic amino acid, an N-methylated amino acid, or a substitute thereof. In some embodiments, X8 is V. In some embodiments, X8 is phenylalanine, tryptophan, tyrosine, or a variant thereof. In some embodiments, X8 is phenylalanine or a variant thereof. In some embodiments, X8 is tryptophan or a variant thereof. In some embodiments, X8 is W1Me. In some embodiments, X8 is tyrosine or a variant thereof. In some embodiments, X8 is N-methylated amino acid or a substitute thereof. In some embodiments, X8 is N-alkylated amino acid or a substitute thereof. In some embodiments, X8 is KCOpipzaa. In some embodiments, X8 is K. In some embodiments, X8 is valine (V). In some embodiments, X8 is Qglucamine. In some embodiments, X8 is Cit. In some embodiments, X8 is hCit. In some embodiments, X8 is absent. In some embodiments of Formulas (I), (I-1), (I-2), (I-3), (I-4), (I-5), (Ia), (Ib), (Ic), (III-1), (III-2), (III-1-RI), and (III-2-RI), X8 is a hydrophobic amino acid, a hydrophilic amino acid, an N-methylated amino acid, or an amino acid with a functional side chain. In some embodiments; X8 is G substituted with one or two straight or branched C₁₋₅ alkyl, A substituted with C₃₋₇ cycloalkyl, or a hydrophilic amino acid wherein the hydrophilic amino acid comprises an L-amino acid comprising —NH₂, one or more —OH, —C(O)OH, —NHC(NH)NH₂, —NHC(O)NH₂, —C(O)NH₂, —NHC(O)CH₃; or the hydrophilic amino acid comprises a zwitterion. In some embodiments, X8 is V, A, E, N, K, Qglucamine, KCOpipzaa, Q, Hse, N, Cit, Hcit, Kac, DapAc, OrAc, T, alT, Aib, Alb, or 3Py6NH2. In some embodiments, X8 is A, E, N, K, Qglucamine, KCOpipzaa, Q, Hse, N, Cit, Hcit, Kac, DapAc, OrnAc, T, alT, Aib, Alb, or 3Py6NH2. In some embodiments, X8 is KCOpipzaa, N, Cit, Qglucamine, hCit, K, KAc, Aib, Alb, DapAc, OrnAc, A, T, alT, Norleucine, Norvaline, Hgl, E, Hgn, Q, I, or L. In certain embodiments, X8 is KCOpipzaa, V, Qglucamine, Cit, Hcit, K, or 3Py6NH2. In certain embodiments, X8 is KCOpipzaa, Qglucamine, Cit, Hcit, K, or 3Py6NH2. In some embodiments, X8 is V, KCOpipzaa, Cit, Qglucamine, hCit, Aib, Alb, Norleucine, or Norvaline. In some embodiments, X8 is KCOpipzaa, Cit, Qglucamine, hCit, Aib, Alb, Norleucine, or Norvaline. In some embodiments, X8 is KCOpipzaa, N, Cit, hCit, KAc, DapAc, OrnAc, A, T, alT, Aib, Alb, Qglucamine, Hgl, Q, E, Hgn, or K. In some embodiments, X8 is a hydrophobic amino acid. In some embodiments, X8 is G substituted with straight or branched C₁₋₅ alkyl. In some embodiments, X8 is G substituted with one or more substituents selected from methyl, ethyl, propyl, isopropyl, butyl, isobutyl, pentyl, and isopentyl. In some embodiments, X8 A substituted with C₃₋₇ cycloalkyl. In some embodiments, X8 is A substituted with cyclopropyl. In some embodiments, X8 is A substituted with cyclobutyl. In some embodiments, X8 is A substituted with cyclopentyl. In some embodiments, X8 is A substituted with cyclohexyl. In some embodiments, X8 is A substituted with cycloheptyl. In some embodiments, X8 is V, Aib, Alb, Norleucine, or Norvaline. In some embodiments, X8 is Aib, Alb, Norleucine, or Norvaline. In some embodiments, X8 is V. In some embodiments, X8 is Aib. In some embodiments, X8 is Alb. In some embodiments, X8 is Norleucine. In some embodiments, X8 is Norvaline. In some embodiments, X8 is a hydrophilic amino acid. In some embodiments, X8 is an amino acid comprising —NH₂, one or more —OH, —C(O)OH, —NHC(NH)NH₂, —NHC(O)NH₂, —C(O)NH₂, or —NHC(O)CH₃. In some embodiments, X8 is an L-amino acid comprising —NH₂, one or more —OH, —C(O)OH, —NHC(NH)NH₂, —NHC(O)NH₂, —C(O)NH₂, or —NHC(O)CH₃. In some embodiments, X8 is an amino acid comprising a zwitterion. In some embodiments, X8 is Cit or a variant thereof. In some embodiments, X8 is Cit or hCit. In some embodiments, X8 is KCOpipzaa. In some embodiments, X8 is Qglucamine. In some embodiments, the metal chelator or linker is attached to X8. In some embodiments, covalently bound radionuclide or linker is attached to X8. In some embodiments, X1 is directly bound to X8.

In some embodiments of Formulas (I), (I-1), (I-2), (I-3), (I-4), (I-5), (Ia), (Ib), (Ic), (III-1), (III-2), (III-1-RI), and (III-2-RI), X9 is W or a variant thereof. In some embodiments, X9 is a canonical amino acid. In some embodiments, X9 is an unnatural amino acid. In some embodiments, X9 is N-alkylated amino acid. In some embodiments, X9 is W1Me, W1Me7Cl, F23dMe, Nal1, Nal2, W1Et, Nal21N, 3Bzf, 3Bzt, Nal15N, Nal14N, Nal24N, Nal28N, F23dC, or W1Me7N. In some embodiments, X9 is W1Me or F23dMe. In some embodiments, X9 is W1Me. In some embodiments, X9 is W1Me7Cl. In some embodiments, X9 is W1Me7N. In some embodiments, X9 is absent. In some embodiments, X9 is F23dMe. In some embodiments, X9 an amino acid having aromatic ring or a substitute thereof. In some embodiments of Formulas (I), (I-1), (I-2), (I-3), (I-4), (I-5), (Ia), (Ib), (Ic), (III-1), (III-2), (III-1-RI), and (III-2-RI), X9 is an amino acid comprising an aromatic ring. In some embodiments, X9 is an amino acid comprising an aryl group. In some embodiments, X9 is an amino acid comprising an optionally substituted phenyl group. In some embodiments, X9 is an amino acid comprising an optionally substituted naphthyl group. In some embodiments, X9 is an amino acid comprising a heteroaryl group. In some embodiments, X9 is an amino acid comprising an optionally substituted monocyclic heteroaryl group. In some embodiments, X9 is an amino acid comprising an optionally substituted bicyclic heteroaryl group. In some embodiments, the aryl or heteroaryl is optionally substituted with 1, 2, or 3 substituents independently selected from —CH₃, -ethyl, —Cl, and —F. In some embodiments, the aryl or heteroaryl is optionally substituted with 1, 2, or 3 substituents independently selected from —OH, oxo, halogen, CN, amino, C₁-C₆ alkyl, C₁-C₆ alkoxyl, and C₁-C₆ haloalkyl. In some embodiments, X9 is W, Y, or a variant thereof (such as an amino acid having either a 6-membered aryl or heteroaryl, or a 9- or 10-membered bi-cyclic aryl or heteroaryl linked to the alpha-carbon through a carbon (e.g., a methylene group), wherein the 6-, 9-, and 10-membered heteroaryl has one heteroatom (e.g., N), and wherein the 6-, 9-, and 10-membered aryl or heteroaryl is optionally substituted by 1 or 2 substituents independently selected from —CH₃, -ethyl, —Cl, and —F). In some embodiments, X9 is an amino acid comprising an aromatic ring (e.g., W or a variant thereof). In some embodiments, X9 is F or a variant thereof, or W or a variant thereof. In some embodiments, X9 is W1Me, W1Me7Cl, W1Me7N, F23dMe, W1Et, W7Me, W, F, or 7-AzaTrp. In some embodiments, X9 is F or a variant thereof. In some embodiments, X9 is F or F23dMe. In some embodiments, X9 is F. In some embodiments, X9 is F23dMe. In some embodiments, X9 is W or a variant thereof. In some embodiments, X9 is W1Me, W1Me7Cl, W1Me7N, W, 7-AzaTrp, W7Me, or W1Et. In some embodiments, X9 is W1Me or F23dMe. In some embodiments, X9 is W1Me. In some embodiments, X9 is W1Me7Cl. In some embodiments, X9 is W1Me7N. In some embodiments, X9 is W. In some embodiments, X9 is 7-AzaTrp. In some embodiments, X9 is W7Me. In some embodiments, X9 is W1Et. In some embodiments, the metal chelator or linker is attached to X9. In some embodiments, the covalently bound radionuclide or linker is attached to X9. In some embodiments, X1 is directly bound to X9.

In some embodiments of Formulas (I), (I-1), (I-2), (I-3), (I-4), (I-5), (III-1), (III-2), (III-1-RI), and (III-2-RI), X10 is absent, T or a variant thereof. In some embodiments, X10 is a canonical amino acid. In some embodiments, X10 is an unnatural amino acid. In some embodiments, X10 is threonine (T). In some embodiments, X10 is absent. In some embodiments of Formulas (I), (I-1), (I-2), (I-3), (I-4), (I-5), (III-1), (III-2), (III-1-RI), and (III-2-RI), X10 is absent, or a polar amino acid (e.g., T or a variant thereof). In some embodiments, X10 is absent, Q, Hgn, S or a variant thereof, T or variant thereof optionally substituted with straight or branched C₁₋₅ alkyl, K or a variant thereof, Cit or a variant thereof, or an L-amino acid substituted with —NHC(NH)NH₂, —NHC(O)NH₂, —C(O)NH₂, or —NHC(O)CH₃. In some embodiments, X10 is absent, T, Q, S, Hgn, Alpha-methylserine, hSer, hThr, N, OrnAc, LysAc, Cit, or hCit. In some embodiments, X10 is absent. In some embodiments, X10 is a polar amino acid. In some embodiments, X10 is Q. In some embodiments, X10 is Hgn. In some embodiments, X10 is S or a variant thereof. In some embodiments. X10 is S, Alpha-methylserine, or hSer. In some embodiments, X10 is S. In some embodiments, X10 is Alpha-methylserine. In some embodiments, X10 is hSer. In some embodiments, X10 is T or a variant thereof optionally substituted with straight or branched C₁₋₅ alkyl. In some embodiments, X10 is T or hThr. In some embodiments, X10 is T. In some embodiments, X10 is hThr. In some embodiments, X10 is T substituted with methyl, ethyl, propyl, isopropyl, butyl, isobutyl, pentyl, or isopentyl. In some embodiments, X10 is N. In some embodiments, X10 is K or a variant thereof. In some embodiments, X10 is K, OrnAc, or LysAc. In some embodiments, X10 is K. In some embodiments, X10 is OrnAc. In some embodiments, X10 is LysAc. In some embodiments, X10 is Cit or a variant thereof. In some embodiments, X10 is Cit or hCit. In some embodiments, X10 is Cit. In some embodiments, X10 is hCit. In some embodiments, the metal chelator or linker is attached to X10. In some embodiments, the covalently bound radionuclide or linker is attached to X10. In some embodiments, X1 is directly bound to X10.

In some embodiments of Formulas (I), (I-1), (I-2), (I-3), (I-4), (I-5), (III-1), (III-2), (III-1-RI), and (III-2-RI), X11 is absent, a hydrophilic amino acid, or a substitute thereof. In some embodiments, X11 is serine, threonine, tyrosine, asparagine, glutamine, or a substitute thereof. In some embodiments, X11 is a canonical amino acid. In some embodiments, X11 is an unnatural amino acid. In some embodiments, X11 is Hgn. In some embodiments, X11 is K. In some embodiments, X11 is glutamate. In some embodiments, X11 is hArg. In some embodiments, X11 is hCit. In some embodiments, X11 is Nmm. In some embodiments, X11 is Ndm. In some embodiments, X11 is Har. In some embodiments, X11 is R. In some embodiments, X11 is Har. In some embodiments, X11 is Arg (R). In some embodiments, X11 is Cit. In some embodiments, X11 is asparagine. In some embodiments, X11 is absent. In some embodiments of Formulas (I), (I-1), (I-2), (I-3), (I-4), (I-5), (III-1), (III-2), (III-1-RI), and (III-2-RI), X11 is absent, a hydrophilic amino acid, or an amino acid with a functional side chain. In some embodiments, X11 is a hydrophilic amino acid. In some embodiments of Formulas (I), (I-1), (I-2), (I-3), (I-4), (I-5), (III-1), (III-2), (III-1-RI), and (III-2-RI), X11 is an amino acid comprising an electrically charged side chain (e.g., E, Hgl, D, R, hArg, K or a variant thereof), or an amino acid comprising a polar uncharged side chain (e.g., Q, Cit, Hgn, N, or a variant thereof). In some embodiments, X11 is an amino acid comprising an electrically charged side chain. In some embodiments, X11 is an amino acid comprising a polar uncharged side chain. In some embodiments, X11 is an amino acid comprising —OH, —NH₂, —C(O)OH, —NHC(═NH)NH₂, —NHC(O)NH₂, —C(O)NH₂, or —NHC(O)CH₃ group. In some embodiments, X11 has an electrically charged side chain. In some embodiments, X11 has a positively charged side chain. In some embodiments, X11 has a negatively charged side chain. In some embodiments, X11 is zwitterionic. In some embodiments, X11 comprises a —OH, —COOH, —NH— or NH₂ moiety. In some embodiments, X11 comprises —OH, —C(O)OH, —NHC(═NH)NH₂, —NHC(O)NH₂, —C(O)NH₂, or —NHC(O)CH₃. In some embodiments, X11 comprises a side chain of C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl. In some embodiments, X11 is absent, E, Hgn, R or a variant thereof, Cit or a variant thereof, Hgl, K or a variant thereof, D, N, or Q. In some embodiments, X11 is absent, E, Hgn, R, hArg, Cit, hCit, Hgl, Orn, D, N, Q, DapAc, OrnAc, DabAc, or norCit. In some embodiments, X11 is absent, arginine (R), asparagine (N), aspartate (D), glutamine (Q), lysine (K), or an unnatural hydrophilic amino acid. In some embodiments, X11 is absent, Hgn, R, hArg, Cit, hCit, Hgl, Orn, D, N, Q, DapAc, OrnAc, DabAc, or norCit. In some embodiments, X11 is Hgn, R, hArg, Cit, hCit, Hgl, Orn, D, N, Q, DapAc, OrnAc, DabAc, or norCit. In some embodiments, X11 is Q, K, G, S, T, E, Aib, Hcit, Cit, Hgn, KCOpipzaa, Har, Nmm, Ndm, Ala, Hgl, 3Py6NH2, or a variant thereof including D-amino acid such as da and variations such as Qglucamine. In some embodiments, X11 is Q, K, G, S, T, Aib, Hcit, Cit, Hgn, KCOpipzaa, Har, Nmm, Ndm, Ala, Hgl, 3Py6NH2, or a variant thereof including D-amino acid such as da and variations such as Qglucamine. In some embodiments, X11 is Hgn, N, R, Har, Nmm, Ndm, E, or K. In some embodiments, X11 is Hgn, N, R, Har, Nmm, Ndm, or K. In some embodiments, X11 is absent. In some embodiments, X11 is a hydrophilic amino acid. In some embodiments, X11 is E. In some embodiments, X11 is Hgn. In some embodiments, X11 is R or a variant thereof. In some embodiments, X11 is R or hArg. In some embodiments, X11 is R. In some embodiments, X11 is hARg. In some embodiments, X11 is Cit or a variant thereof. In some embodiments, X11 is Cit, hCit, or norCit. In some embodiments, X11 is Cit. In some embodiments, X11 is hCit. In some embodiments, X11 is norCit. In some embodiments, X11 is Hgl. In some embodiments, X11 is K or a variant thereof. In some embodiments, X11 is K, Orn, OrnAc, DabAc, or DapAc. In some embodiments, X11 is K. In some embodiments, X11 is Orn. In some embodiments, X11 is OrnAc. In some embodiments, X11 is DabAc. In some embodiments, X11 is DapAc. In some embodiments, X11 is D, N or Q. In some embodiments, X11 is D. In some embodiments, X11 is N. In some embodiments, X11 is Q. In some embodiments, the metal chelator or linker is attached to X11. In some embodiments, the covalently bound radionuclide or linker is attached to X11. In some embodiments, X1 is directly bound to X11.

In some embodiments of Formulas (I), (I-5), (Ia), (Ib), (Ic), (III-2), and (III-2-RI), X12 is C or a variant thereof. In some embodiments, X12 is a canonical amino acid. In some embodiments, X12 is an unnatural amino acid. In some embodiments, X12 is cysteine. In some embodiments, X12 is a substitute of cysteine. In some embodiments, X12 is homocysteine. In some embodiments, X12 is CdMe. In some embodiments, X12 is C3SMe. In some embodiments, X12 is C3RMe. In some embodiments, the metal chelator or linker is attached to X12. In some embodiments of Formulas (I), (I-5), (Ia), (Ib), (Ic), (III-2), and (III-2-RI), X12 is C or a variant thereof. In some embodiments, X12 is X12 is C, hCys, CdMe, C3RMe, C3SMe, Selenocysteine, dc, or Penicillamine. In some embodiments, X12 is C. In some embodiments, X12 is hCys. In some embodiments, X12 is CdMe. In some embodiments, X12 is C3RMe. In some embodiments, X12 is C3SMe. In some embodiments, X12 is Selenocysteine. In some embodiments, X12 is dc. In some embodiments, X12 is Penicillamine. In some embodiments, the metal chelator or linker is attached to X12. In some embodiments, the covalently bound radionuclide or linker is attached to X12. In some embodiments, X1 is directly bound to X12.

In some embodiments, the peptide of Formula (I) has a structure of Formula (I-1), or a pharmaceutically acceptable salt thereof,

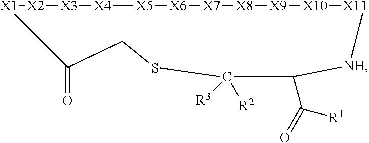

wherein
   R¹ is selected from the group consisting of NH₂ and OH;
   R² is selected from the group consisting of H or C₁₋₃ alkyl;
   R³ is selected from the group consisting of H or C₁₋₃ alkyl;
wherein the attachment point to the metal chelator or the linker is not shown, and wherein X1-X11 are described in Formula (I).

In some embodiments, the peptide of Formula (I-1) has a structure of Formula (I-2), or a pharmaceutically acceptable salt thereof,

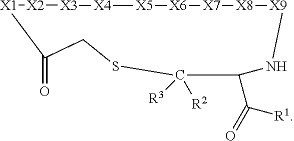

In some embodiments, the peptide of Formula (I-1) has a structure of Formula (I-3), or a pharmaceutically acceptable salt thereof,

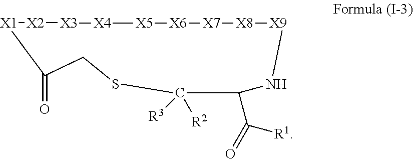

In some embodiments, the peptide of Formula (I-1) has a structure of Formula (I-4), or a pharmaceutically acceptable salt thereof,

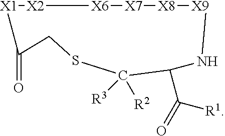

In some embodiments of Formula (I-1), (I-2), (I-3) or (I-4), R¹ is OH. In some embodiments of Formula (I-1), (I-2), (I-3) or (I-4), R¹ is NH₂. In some embodiments of Formula (I-1), (I-2), (I-3) or (I-4), R¹ is attached to the linker or to the metal chelator. In some embodiments, the linker or the metal chelator is attached to the peptide through the group R¹.

In some embodiments of Formula (I-1), (I-2), (I-3) or (I-4), R² is H. In some embodiments of Formula (I-1), (I-2), (I-3) or (I-4), R² is C₁₋₃ alkyl. In some embodiments of Formula (I-1), (I-2), (I-3) or (I-4), R² is methyl.

In some embodiments of Formula (I-1), (I-2), (I-3) or (I-4), R³ is H. In some embodiments of Formula (I-1), (I-2), (I-3) or (I-4), R³ is C₁₋₃ alkyl. In some embodiments of Formula (I-1), (I-2), (I-3) or (I-4), R³ is methyl.

In some embodiments, the peptide of Formula (I) has a structure of Formula (I-5), or a pharmaceutically acceptable salt thereof,

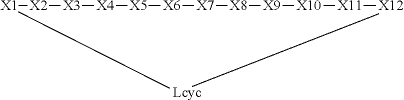

wherein X1-X12 have the definition described above and Lcyc is a ring closing group that covalently connecting X1 with X12.

In some embodiments, the Lcyc is a group selected from Table 4B. In some embodiments, the Lcyc is formed by reacting the first and the second functional groups in Table 4C.

In some embodiments, the peptide of Formula (I) or a pharmaceutically acceptable salt thereof,

X1-X2-X3-X4-X5-X6-X7-X8-X9-X10-X11-X12 Formula (I)
wherein
   X1 is any amino acid (e.g., D-amino acid);
   X2 is an amino acid comprising an aromatic ring or a variant thereof, or N-methylated amino acid thereof;
   X3 is N or a variant thereof;
   X4 is any hydrophobic amino acid or a variant thereof;
   X5 is a hydrophilic amino acid or a variant thereof;
   X6 is a hydrophilic amino acid or amino acid having aromatic ring, or N-methylated amino acid thereof;
   X7 is W or a variant thereof;
   X8 is V or hydrophilic amino acid or a variant thereof;
   X9 is W or a variant thereof;
   X10 is T or a variant thereof;
   X11 is any hydrophilic amino acid; and
   X12 is C or a variant thereof.

In some embodiments of Formula (I), wherein,
X1 is D-amino acid (such as da, df3CON, dahp, or dkCOpipzaa);
X2 is N-methylated phenylalanine or a variant thereof (such as Me3Py, MeF, MeF3H, or MeF3CN);
X3 is N;
X4 is a hydrophobic amino acid or N-methylated amino acid (such as leucine, Cbg, or Chg);
X5 is a Hgn, asparagine (N), 2,4-Diaminobutyric Acid (Dab), Qglucamine, KCOpipzaa, Hgl, Nmm, Ndm, or lysine (K);
X6 is asparagine (N) or N-methylated glutamic acid (E), N-methylated asparagine, N-methylated phenylalanine (F) or substitutions thereof (such as Qglucamine, MeE, MEN, Me3Py, MeF, MeF4C, or N);
X7 is W1Me, W1Me7Cl, or W1Me7N;
X8 is KCOpipzaa, V, Qglucamine, Cit, Hcit, or K;
X9 is W1Me or F23dMe;
X10 is T;
X11 is hArg, hCit, Citrulline (Cit), A Hgn, asparagine (N), Arginine (R), Har, Nmm, Ndm, Glutamic Acid (E), lysine (K); and
X12 is cysteine.

In some embodiments, an amino acid of Formula (I) has a sequence of Formula (Ia), or a pharmaceutically acceptable salt thereof,

X1-X2-X3-X4-X5-X6-X7-X8-X9-X12 Formula (Ia).

In some embodiments, an amino acid of Formula (I) has a sequence of Formula (Ib), or a pharmaceutically acceptable salt thereof,

X1-X2-X4-X5-X7-X8-X9-X12 Formula (Ib).

In some embodiments, an amino acid of Formula (I) has a sequence of Formula (Ic), or a pharmaceutically acceptable salt thereof,

X1-X2-X6-X7-X8-X9-X12 Formula (Ic).

In some embodiments, a herein described peptide has an amino acid sequence according to Formula (Ia), or a pharmaceutically acceptable salt thereof,

X1-X2-X3-X4-X5-X6-X7-X8-X9-X12 Formula (Ia)
wherein,
   X1 is any amino acid (e.g., a D-amino acid);
   X2 is an amino acid comprising an aromatic ring or a variant thereof, or an N-methylated amino acid thereof;
   X3 is N or a variant thereof;
   X4 is any hydrophobic amino or a variant thereof,
   X5 is a hydrophilic amino acid or a variant thereof;
   X6 is a hydrophilic amino acid or amino acid having aromatic ring, or an N-methylated amino acid thereof;
   X7 is W or a variant thereof;
   X8 is any hydrophilic amino acid or a variant thereof;
   X9 is W or a variant thereof; and
   X12 is C or a variant thereof.

In some embodiments, a herein described peptide has an amino acid sequence according to Formula (Ib), or a pharmaceutically acceptable salt thereof,

X1-X2-X4-X5-X7-X8-X9-X12 Formula (Ib)
wherein,
   X1 is any amino acid (e.g., D-amino acid);
   X2 is an amino acid comprising an aromatic ring or a variant thereof, or N-methylated amino acid thereof;
   X4 is any hydrophobic amino or a variant thereof, X5 is a hydrophilic amino acid or a variant thereof;
   X7 is W or a variant thereof;
   X8 is an N-methylated amino acid;
   X9 is W or a variant thereof; and
   X12 is C or a variant thereof.

In some embodiments, a herein described peptide has an amino acid sequence according to Formula (Ic), or a pharmaceutically acceptable salt thereof,

X1-X2-X6-X7-X8-X9-X12 Formula (Ic)
wherein,
   X1 is any amino acid (e.g., D-amino acid);
   X2 is an amino acid comprising an aromatic ring or a variant thereof, or N-methylated amino acid thereof;
   X6 is an N-methyl amino acid;
   X7 is W or a variant thereof;
   X8 is an N-methyl amino acid;
   X9 is W or a variant thereof; and
   X12 is C or a variant thereof.

In some embodiments, the peptide of Formula (I), (Ia), (Ib), and/or (Ic) are monocyclic. In some embodiments, the amino acid in X1 and the cysteine or the substitution of cysteine are bound.

In some embodiments, a peptide of the present disclosure binds to a ligand-binding domain (LBD) of human EphA2.

In some embodiments, a peptide of the present disclosure has good contact with Asp53 and/or Glu157 of the human EphA2, according to SEQ ID NO: 276. In some embodiments, a peptide of the present disclosure interacts with Asp53 and/or Glu157 of the human EphA2, according to SEQ ID NO: 276. In some embodiments, a peptide of the present disclosure interacts with Asp53 and/or Glu157 of the human EphA2, according to SEQ ID NO: 501. The interaction can be the formation of one or more hydrogen bonds, Van der Waals interactions, dipole-dipole interactions, or pi-pi stacking interactions. In some embodiments, a peptide of the present disclosure interacts with human EphA2 at one or more residues selected from Asp53, Met55, Asn57, Met59, Met66, Thr101, Arg103, Phe156, Glu157, Arg159, Val161, Val189, and Ala190. In some embodiments, a peptide of the present disclosure binds to Asp53 and Glu157 of the human EphA2. In some embodiments, amino acid residue X5 of Formula (I) interact with Glu157 of a human EphA2. In some embodiments, amino acid residue X6 of Formula (I) interacts with Arg 159 of a human EphA2. In some embodiments, amino acid residue X7 of Formula (I) interacts with one or more of Phe156, Thr101, Asn57, Val161, Met59, Ala190, and Met66 of a human EphA2. In some embodiments, amino acid residue X9 of Formula (I) interacts with one or more of Phe156, Arg103, and Val189. In some embodiments, amino acid residue X11 of Formula (I) interact with Asp53 of a human EphA2. In some embodiments, amino acid residue X7 of Formula (I) forms a pi-pi stacking interaction with Phe156 of a human EphA2 of the human EphA2. In some embodiments, amino acid residue X9 of Formula (I) forms a pi-pi stacking interaction with Phe156 of a human EphA2. In some embodiments, amino acid residue X2 of Formula (I) interacts with the backbone carbonyl of C70 of human EphA2 protein via intermolecular aromatic H-bond interactions.

In some embodiments, when a peptide of Formula (I), or a conjugate comprising the peptide, is bound to a human EphA2, amino acid residue X2 of Formula (I) is located less than 15 Å from the C70 of the human EphA2. In some embodiments, X2 is located less than 10 Å from the C70. In some embodiments, X2 is located less than 6 Å from the C70. In some embodiments, X2 is located less than 4 Å from the C70.

In some embodiments, when a peptide of Formula (I), or a conjugate comprising the peptide, is bound to a human EphA2, amino acid residue X7 of Formula (I) is located less than 10 Å from the Phe156 of the human EphA2. In some embodiments, X7 is located less than 6 Å from the Phe156. In some embodiments, X7 is located less than 4 Å from the Phe156.

In some embodiments, when a peptide of Formula (I), or a conjugate comprising the peptide, is bound to a human EphA2, amino acid residue X7 of Formula (I) is located less than 20 Å from the Thr101 of the human EphA2. In some embodiments, X7 is located less than 15 Å from the Thr101. In some embodiments, X7 is located less than 10 Å from the Thr101. In some embodiments, X7 is located less than 6 Å from the Thr101. In some embodiments, X7 is located less than 4 Å from the Thr101.

In some embodiments, when a peptide of Formula (I), or a conjugate comprising the peptide, is bound to a human EphA2, amino acid residue X7 of Formula (I) is located less than 20 Å from the Asn57 of the human EphA2. In some embodiments, X7 is located less than 15 Å from the Asn57. In some embodiments, X7 is located less than 10 Å from the Asn57. In some embodiments, X7 is located less than 6 Å from the Asn57. In some embodiments, X7 is located less than 4 Å from the Asn57.

In some embodiments, when a peptide of Formula (I) is, or a conjugate comprising the peptide, bound to a human EphA2, amino acid residue X7 of Formula (I) is located less than 20 Å from the Val161 of the human EphA2. In some embodiments, X7 is located less than 15 Å from the Val161. In some embodiments, X7 is located less than 10 Å from the Val161. In some embodiments, X7 is located less than 6 Å from the Val161. In some embodiments, X7 is located less than 4 Å from the Val161.

In some embodiments, when a peptide of Formula (I), or a conjugate comprising the peptide, is bound to a human EphA2, amino acid residue X7 of Formula (I) is located less than 20 Å from the Met59 of the human EphA2. In some embodiments, X7 is located less than 15 Å from the Met59. In some embodiments, X7 is located less than 10 Å from the Met59. In some embodiments, X7 is located less than 6 Å from the Met59. In some embodiments, X7 is located less than 4 Å from the Met59.

In some embodiments, when a peptide of Formula (I), or a conjugate comprising the peptide, is bound to a human EphA2, amino acid residue X7 of Formula (I) is located less than 20 Å from the Ala190 of the human EphA2. In some embodiments, X7 is located less than 15 Å from the Ala190. In some embodiments, X7 is located less than 10 Å from the Ala190. In some embodiments, X7 is located less than 6 Å from the Ala190. In some embodiments, X7 is located less than 4 Å from the Ala190.

In some embodiments, when a peptide of Formula (I), or a conjugate comprising the peptide, is bound to a human EphA2, amino acid residue X7 of Formula (I) is located less than 20 Å from the Met66 of the human EphA2. In some embodiments, X7 is located less than 15 Å from the Met66. In some embodiments, X7 is located less than 10 Å from the Met66. In some embodiments, X7 is located less than 6 Å from the Met66. In some embodiments, X7 is located less than 4 Å from the Met66.

In some embodiments, when a peptide of Formula (I), or a conjugate comprising the peptide, is bound to a human EphA2, amino acid residue X9 of Formula (I) is located less than 10 Å from the Phe156 of the human EphA2. In some embodiments, X9 is located less than 6 Å from the Phe156. In some embodiments, X9 is located less than 4 Å from the Phe156.

In some embodiments, when a peptide of Formula (I), or a conjugate comprising the peptide, is bound to a human EphA2, amino acid residue X9 of Formula (I) is located less than 15 Å from the Asn3 of the human EphA2. In some embodiments, X9 is located less than 10 Å from the Asn3. In some embodiments, X9 is located less than 6 Å from the Asn3. In some embodiments, X9 is located less than 4 Å from the Asn3.

In some embodiments, when a peptide of Formula (I), or a conjugate comprising the peptide, is bound to a human EphA2, amino acid residue X9 of Formula (I) is located less than 15 Å from the Arg 103 of the human EphA2. In some embodiments, X9 is located less than 10 Å from the Arg103. In some embodiments, X9 is located less than 6 Å from the Arg103. In some embodiments, X9 is located less than 4 Å from the Arg103.

In some embodiments, when a peptide of Formula (I), or a conjugate comprising the peptide, is bound to a human EphA2, amino acid residue X9 of Formula (I) is located less than 15 Å from the Val189 of the human EphA2. In some embodiments, X9 is located less than 10 Å from the Val189. In some embodiments, X9 is located less than 6 Å from the Val189. In some embodiments, X9 is located less than 4 Å from the Val189.

In some embodiments, when a peptide of Formula (I), or a conjugate comprising the peptide, is bound to a human EphA2, amino acid residue X8 of Formula (I) is located less than 10 Å from the Phe156 of the human EphA2. In some embodiments, X8 is located less than 6 Å from the Phe156. In some embodiments, X8 is located less than 4 Å from the Phe156.

In some embodiments, when a peptide of Formula (I), or a conjugate comprising the peptide, is bound to a human EphA2, amino acid residue X2 of Formula (I) is located less than 15 Å from the C70 of the human EphA2. In some embodiments, X2 is located less than 10 Å from the C70. In some embodiments, X2 is located less than 7 Å from the C70. In some embodiments, X2 is located less than 4 Å from the C70.

In some embodiments, when a peptide of Formula (I), or a conjugate comprising the peptide, is bound to a human EphA2, amino acid residue X7 of Formula (I) is located less than 10 Å from the Phe156 of the human EphA2. In some embodiments, X7 is located less than 6 Å from the Phe156. In some embodiments, X7 is located less than 3 Å from the Phe156.

In some embodiments, when a peptide of Formula (I), or a conjugate comprising the peptide, is bound to a human EphA2, amino acid residue X9 of Formula (I) is located less than 20 Å from the Thr101 of the human EphA2. In some embodiments, X9 is located less than 15 Å from the Thr101. In some embodiments, X9 is located less than 10 Å from the Thr101. In some embodiments, X9 is located less than 6 Å from the Thr101. In some embodiments, X9 is located less than 5 Å from the Thr101.

In some embodiments, when a peptide of Formula (I), or a conjugate comprising the peptide, is bound to a human EphA2, amino acid residue X8 of Formula (I) is located less than 20 Å from the Asn57 of the human EphA2. In some embodiments, X8 is located less than 15 Å from the Asn57. In some embodiments, X8 is located less than 10 Å from the Asn57. In some embodiments, X8 is located less than 6 Å from the Asn57. In some embodiments, X8 is located less than 4 Å from the Asn57.

In some embodiments, when a peptide of Formula (I) is, or a conjugate comprising the peptide, bound to a human EphA2, amino acid residue X7 of Formula (I) is located less than 20 Å from the Val161 of the human EphA2. In some embodiments, X7 is located less than 15 Å from the Val161. In some embodiments, X7 is located less than 11 Å from the Val161. In some embodiments, X7 is located less than 6 Å from the Val161. In some embodiments, X7 is located less than 5 Å from the Val161.

In some embodiments, when a peptide of Formula (I), or a conjugate comprising the peptide, is bound to a human EphA2, amino acid residue X7 of Formula (I) is located less than 20 Å from the Met59 of the human EphA2. In some embodiments, X7 is located less than 15 Å from the Met59. In some embodiments, X7 is located less than 11 Å from the Met59. In some embodiments, X7 is located less than 6 Å from the Met59. In some embodiments, X7 is located less than 4 Å from the Met59.

In some embodiments, when a peptide of Formula (I), or a conjugate comprising the peptide, is bound to a human EphA2, amino acid residue X7 of Formula (I) is located less than 20 Å from the Ala190 of the human EphA2. In some embodiments, X7 is located less than 15 Å from the Ala190. In some embodiments, X7 is located less than 11 Å from the Ala190. In some embodiments, X7 is located less than 6 Å from the Ala190. In some embodiments, X7 is located less than 4 Å from the Ala190.

In some embodiments, when a peptide of Formula (I), or a conjugate comprising the peptide, is bound to a human EphA2, amino acid residue X7 of Formula (I) is located less than 20 Å from the Met66 of the human EphA2. In some embodiments, X7 is located less than 15 Å from the Met66. In some embodiments, X7 is located less than 10 Å from the Met66. In some embodiments, X7 is located less than 6 Å from the Met66. In some embodiments, X7 is located less than 4 Å from the Met66.

In some embodiments, when a peptide of Formula (I), or a conjugate comprising the peptide, is bound to a human EphA2, amino acid residue X2 of Formula (I) is located less than 15 Å from the Arg 103 of the human EphA2. In some embodiments, X2 is located less than 10 Å from the Arg103. In some embodiments, X2 is located less than 6 Å from the Arg 103. In some embodiments, X2 is located less than 4 Å from the Arg103.

In some embodiments, when a peptide of Formula (I), or a conjugate comprising the peptide, is bound to a human EphA2, amino acid residue X9 of Formula (I) is located less than 15 Å from the Val189 of the human EphA2. In some embodiments, X9 is located less than 10 Å from the Val189. In some embodiments, X9 is located less than 6 Å from the Val189. In some embodiments, X9 is located less than 4 Å from the Val189.

In some embodiments, a conjugate of the present disclosure has a structure of Formula (III-1),

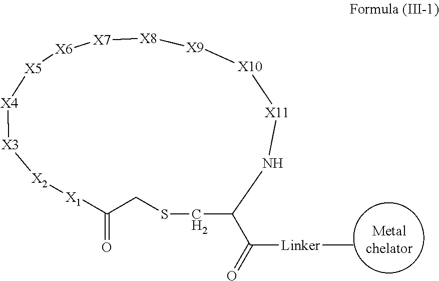

wherein -Linker- represents the linker.

In some embodiments, a conjugate comprising a cyclic peptide of formula (I) has a structure of Formula (III-2),

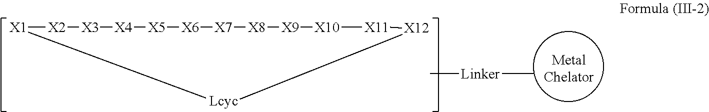

wherein
X1-X12 have the definition described above and Lcyc is a ring closing group that covalently connecting
X1 with X12; and
-Linker- represents the linker.

In some embodiments, a conjugate of the present disclosure has a structure of Formula (III-1-RI),

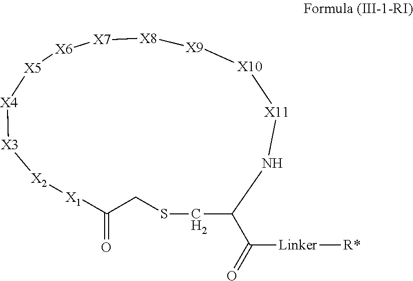

wherein
   X1-X12 have the definition described above;
   -Linker- represents the linker; and
   R* represents the covalently bound radionuclide.

In some embodiments, a conjugate comprising a cyclic peptide of formula (I) has a structure of Formula (III-2-RI),

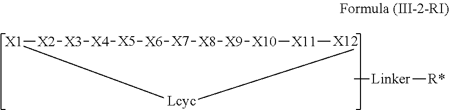

wherein
   X1-X12 have the definition described above and Lcyc is a ring closing group that covalently connecting X1 with X12;
   -Linker- represents the linker; and
   R* represents the covalently bound radionuclide.

In some embodiments, the Lcyc is a group selected from Table 4B. In some embodiments, the Lcyc is formed by reacting the first and the second functional groups in Table 4C. In some embodiments, the Lcyc is —C(═O)—CH₂—. In some embodiments, the Lcyc is —C(═O)—CH₂—, which is formed by reacting with a chloroacetylated (or bromoacetylated) amino acid with a cysteine. In some embodiments, the Lcyc is —C(═O)—CH₂—S—, which is formed by reacting with a chloroacetylated (or bromoacetylated) amino acid with an amino acid comprising a SH group.

In some embodiments, a peptide disclosed herein or a pharmaceutically accepted salt thereof has a cyclic structure having a chloroacetylated amino acid in the first residue X1 and a cysteine residue or a variant thereof, and wherein the chloroacetylated amino acid in X1 and the cysteine residue or a variant thereof are bound. In some embodiments, a peptide disclosed herein or a pharmaceutically accepted salt thereof has a cyclic structure having a chloroacetylated amino acid in the first residue X1 and a cysteine residue or a variant thereof, and wherein the chloroacetylated amino acid in X1 and the cysteine residue or a variant thereof form a covalent bond. In some embodiments, a peptide disclosed herein or a pharmaceutically accepted salt thereof has a cyclic structure having a bromoacetylated amino acid in the first residue X1 and a cysteine residue or a variant thereof, and wherein the bromoacetylated amino acid in X1 and the cysteine residue or a variant thereof form a covalent bond.

In some embodiments, the peptide consists of an amino acid sequence selected from SEQ ID NOs: 1-122, 159-163, and 165-171, and the peptide has a cyclic structure having a cysteine residue or a variant thereof at 12th residue (X12). In some embodiments, the peptide consists of an amino acid sequence selected from SEQ ID NOs: 1-122, 159-163, and 165-171, and the peptide has a cyclic structure having a cysteine residue or a variant thereof at 12th residue (X12), and wherein the chloroacetylated amino acid and the cysteine residue or a variant thereof at 12th residue form a covalent bond. In some embodiments, the chloroacetyl group can be replaced with a bromoacetyl group.

In some embodiments, the peptide consists of an amino acid sequence selected from SEQ ID NOs: 123-149 and 164, and the peptide has a cyclic structure having a cysteine residue or a variant thereof at 10th residue (X10). In some embodiments, the peptide consists of an amino acid sequence selected from SEQ ID NOs: 123-149 and 164, and the peptide has a cyclic structure having a cysteine residue or a variant thereof at 10th residue (X10), and wherein the chloroacetylated amino acid and the cysteine residue or a variant thereof at 10th residue form a covalent bond. In some embodiments, the chloroacetyl group can be replaced with a bromoacetyl group.

In some embodiments, the peptide consists of an amino acid sequence selected from SEQ ID NOs: 150-157, and the peptide has a cyclic structure having a cysteine residue or a variant thereof at 8th residue (X8). In some embodiments, the peptide consists of an amino acid sequence selected from SEQ ID NOs: 150-157, and the peptide has a cyclic structure having a chloroacetylated amino acid and a cysteine residue or a variant thereof at 8th residue (X8), and wherein the chloroacetylated amino acid and the cysteine residue or a variant thereof at 8th residue form a covalent bond. In some embodiments, the chloroacetyl group can be replaced with a bromoacetyl group.

In some embodiments, the peptide consists of an amino acid sequence selected from SEQ ID NO: 158, and the peptide has a cyclic structure having a cysteine residue or a variant thereof at 7th residue (X7). In some embodiments, the peptide consists of an amino acid sequence selected from SEQ ID NO: 158, and the peptide has a cyclic structure having a chloroacetylated amino acid and a cysteine residue or a variant thereof at 7th residue (X7), and wherein the chloroacetylated amino acid and the cysteine residue or a variant thereof at 7th residue form a covalent bond. In some embodiments, the chloroacetyl group can be replaced with a bromoacetyl group.

In some embodiments, a peptide disclosed herein or a pharmaceutically salt thereof has a cyclic structure having the first amino acid covalently linked to the last amino acid.

In some embodiments, the peptide or the pharmaceutically accepted salt thereof has a cyclic structure having a chloroacetylated amino acid in X1 and a cysteine or substituted cysteine residue, and wherein the chloroacetylated amino acid in X1 and the cysteine or substituted cysteine are bound. In some embodiments, the peptide consists of an amino acid sequence selected from SEQ ID NOs: 1-171. In some embodiments, the peptide consists of an amino acid sequence selected from SEQ ID NOs: 1-171, and the peptide has a cyclic structure. In some embodiments, the peptide consists of an amino acid sequence selected from SEQ ID NOs: 1-171, and the peptide has a cyclic structure having a chloroacetylated amino acid and a cysteine or substituted cysteine residue at C-terminus, and wherein the chloroacetylated amino acid and the cysteine or substituted cysteine at C-terminus are bound. In some embodiments, the peptide has a cyclic structure having a chloroacetylated amino acid and; (i) a cysteine or substituted cysteine residue at 12th residue, and wherein the chloroacetylated amino acid and the cysteine or substituted cysteine at 12th residue are bound; or (ii) a cysteine or substituted cysteine residue at 10th residue, and wherein the chloroacetylated amino acid and the cysteine or substituted cysteine at 10th residue are bound. In some embodiments, the chloroacetyl group can be replaced with a bromoacetyl group.

For example, a cyclic peptide of formula (I) can have a structure as illustrated below

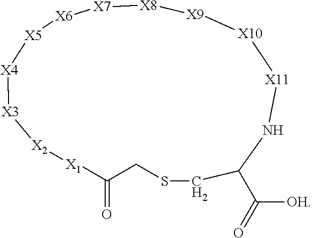

For example, a cyclic peptide of formula (I) can have a structure as illustrated below

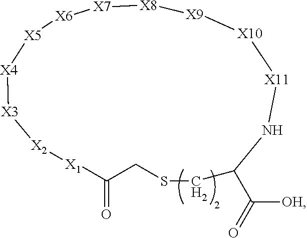

In some embodiments, a conjugate comprising a cyclic peptide of formula (I) has a structure of

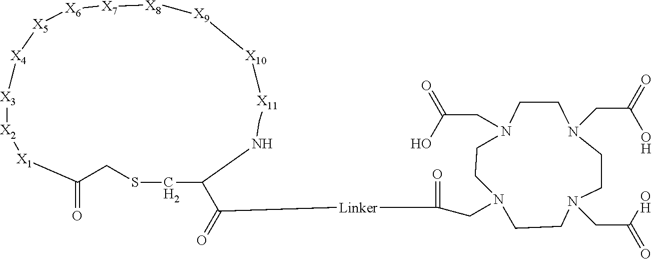

In some embodiments, a conjugate of the present disclosure has a structure of

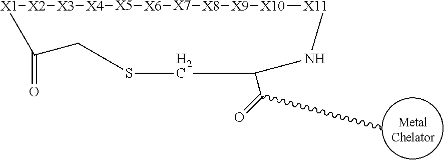

wherein [symbol] represents the linker.

In some embodiments, a conjugate comprising a cyclic peptide of formula (I) has a structure of

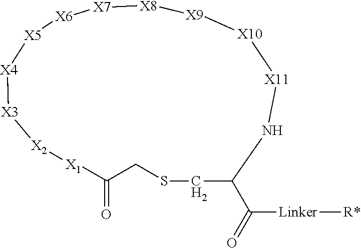

In some embodiments, a conjugate of the present disclosure has a structure of

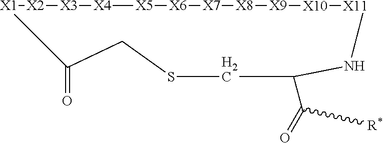

wherein [symbol] represents the linker.

In some embodiments, the peptide or the salt thereof comprises an amino acid sequence that is at least 95% identical to a sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID No: 158. In some embodiments, the peptide or the salt thereof comprises an amino acid sequence that is at least 90% identical to a sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID No: 158. In some embodiments, the peptide or the salt thereof comprises an amino acid sequence that is at least 80%, 85%, 90%, 95%, or 98% identical to a sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID No: 158. In some embodiments, the peptide or the salt thereof consists of an amino acid sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID No: 158. In some embodiments, the peptide or the salt thereof comprises an amino acid sequence that has at most 1, 2, 3, 4, or 5 amino acid residues that are different compared to a sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID No: 158. In some embodiments, the peptide or the salt thereof comprises an amino acid sequence that has at most 1, 2, 3, 4, or 5 additions, deletions and/or substitutions (including conservative substitutions) to a sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID No: 158. In some embodiments, the peptide or the salt thereof comprises an amino acid sequence that has at most 1 addition, deletion, or substitutions (including conservative substitutions) to a sequence selected from SEQ ID NOs: (1) X1-X12 of SEQ ID NOs: 1-122, 159-163, and 165-171, (2) X1-X10 of SEQ ID NOs: 123-149 and 164, (3) X1-X8 of SEQ ID NOs: 150-157, and (4) X1-X7 of SEQ ID No: 158. In some embodiments, the peptide is not SEQ ID NO: 1. In some embodiments, a radiopharmaceutical conjugate described herein comprises a peptide of SEQ ID Nos: 1-275 or 278-449. In some embodiments, the radiopharmaceutical conjugate is not SEQ ID NO: 282.

Exemplary peptides of the present disclosure include the peptides described in Table 1. In some embodiments, the peptides of Table 1 have a —C(═O)-halogen group attached to the N-terminus. In some embodiments, the peptides of Table 1 have a —C(═O)—CH₂-halogen group attached to the N-terminus. In some embodiments, the peptides of Table 1 have a —C(═O)-halogen group attached at residue position 1 (e.g., X1). In some embodiments, the peptides of Table 1 have a —C(═O)—CH₂-halogen group attached at residue position 1 (e.g., X1). In some embodiments, the peptides of Table 1 have a —C(═O)—Cl group attached to the N-terminus. In some embodiments, the peptides of Table 1 have a —C(═O)—CH₂—C₁ group attached to the N-terminus. In some embodiments, the peptides of Table 1 have a —C(═O)—Cl group attached at residue position 1 (e.g., X1). In some embodiments, the peptides of Table 1 have a —C(═O)—CH₂—C₁ group attached at residue position 1 (e.g., X1). In some embodiments, the peptides of Table 1 have a —C(═O)—Br group attached at residue position 1 (e.g., X1). In some embodiments, the peptides of Table 1 have a —C(═O)—CH₂—Br group attached at residue position 1 (e.g., X1).

In some embodiments, the radiopharmaceutical conjugates of the disclosure have a —C(═O)-halogen group attached to the N-terminus. In some embodiments, the radiopharmaceutical conjugates of the disclosure have a —C(═O)—CH₂-halogen group attached to the N-terminus. In some embodiments, the radiopharmaceutical conjugates of the disclosure have a —C(═O)-halogen group attached at residue position 1 (e.g., X1). In some embodiments, the radiopharmaceutical conjugates of the disclosure have a —C(═O)—CH₂-halogen group attached at residue position 1 (e.g., X1). In some embodiments, the radiopharmaceutical conjugates of the disclosure have a —C(═O)—Cl group attached to the N-terminus. In some embodiments, the radiopharmaceutical conjugates of the disclosure have a —C(═O)—CH₂—C₁ group attached to the N-terminus. In some embodiments, the radiopharmaceutical conjugates of the disclosure have a —C(═O)—Cl group attached at residue position 1 (e.g., X1). In some embodiments, the radiopharmaceutical conjugates of the disclosure have a —C(═O)—CH₂—Cl group attached at residue position 1 (e.g., X1). In some embodiments, the radiopharmaceutical conjugates of the disclosure have a —C(═O)—Br group attached at residue position 1 (e.g., X1). In some embodiments, the radiopharmaceutical conjugates of the disclosure have a —C(═O)—CH₂—Br group attached at residue position 1 (e.g., X1). In some embodiments, the peptides in the radiopharmaceutical conjugates of the disclosure are monocyclic.

In some embodiments, the peptides of the radiopharmaceutical conjugates described herein are monocyclic peptides, wherein the —C(═O)—Cl at residue position 1 (e.g., X1) forms a bond with the cysteine at residue position 12 (e.g., X12). In some embodiments, the peptides of the radiopharmaceutical conjugates described herein are monocyclic peptides, wherein the —C(═O)—CH2-Cl at residue position 1 (e.g., X1) forms a bond with the cysteine at residue position 12 (e.g., X12). In some embodiments, the peptides in the radiopharmaceutical conjugates described herein are monocyclic peptides with 12 amino acid residues forming the ring.

Exemplary peptides of the present disclosure include the peptides described in Table 1. In some embodiments, the peptides of Table 1 have an —C(═O)-halogen group attached to the N-terminus. In some embodiments, the peptides of Table 1 have an —C(═O)-halogen group attached at residue position 1 (e.g., X1). In some embodiments, the peptides of Table 1 have an —C(═O)—Cl group attached to the N-terminus. In some embodiments, the peptides of Table 1 have an —C(═O)—Cl group attached at residue position 1 (e.g., X1). In some embodiments, the peptides of Table 1 have an —C(═O)—Br group attached at residue position 1 (e.g., X1).

In some embodiments, a herein described conjugate is selected from conjugates described in Table 2A-Lu, Table 2A-Lu177 or Table 2A-Ac255. In some embodiments, a herein described conjugate is selected from conjugates described in Table 2B, Table 2B-Lu, Table 2B-Lu177 or Table 2B-Ac255. In some embodiments, a herein described conjugate is selected from conjugates described in Table 2C.

In some embodiments, provided herein are conjugates having the same peptide sequence and linker as the conjugates described in Table 2A-Lu, Table 2A-Lu177, Table 2A-Ac255, Table 2B, Table 2B-Lu, Table 2B-Lu177, Table 2B-Ac255, or Table 2C, except that the ring closing linkage between the amino acid residue of position 1 and the cysteine (e.g., at position 10 or 12) are covalently bound by a different group. For example, the amino acid residue of position 1 can comprise a group selected from maleimides, halides, disulfides, electron-deficient alkynes, thioesters, and alkenes, which forms a covalent bond with the cysteine.

In some embodiments, the peptides of conjugates of Table 2A-Lu, Table 2A-Lu177, and Table 2A-Ac255 are monocyclic peptides, wherein the —C(═O)—Cl at residue position 1 forms a bond with the cysteine at residue position 12. In some embodiments, the —C(═O)—CH₂—Cl at residue position 1 forms a bond with the cysteine at residue position 12. In some embodiments, the peptides in the conjugates of Table 2A-Lu, Table 2A-Lu177, and Table 2A-Ac255 are monocyclic peptides with 12 amino acid residues forming the ring. In some embodiments, the peptides of conjugates of Table 2B, Table 2B-Lu, Table 2B-Lu177, and Table 2B-Ac255 are monocyclic peptides, wherein the —C(═O)—Cl at residue position 1 forms a bond with the cysteine at residue position 10. In some embodiments, the —C(═O)—CH₂—Cl at residue position 1 forms a bond with the cysteine at residue position 10. In some embodiments, the peptides in the conjugates of Table 2B, Table 2B-Lu, Table 2B-Lu177, and Table 2B-Ac255 are monocyclic peptides with 10 amino acid residues forming the ring.

In one aspect, described herein is a peptide that has avidity for ephrin type-A receptor 2 (EphA2), wherein the peptide competes for binding to human EphA2 with a peptide that has an amino acid sequence including deletion, substitution, and/or addition of one or several amino acids in the amino acid of SEQ ID NO:1:

(SEQ ID NO: 1)
da-MeF-N-L-Hgl-MeF-W1Me-V-W1Me-T-E-C

or a pharmaceutically acceptable salt thereof.

In one aspect, described herein is a peptide that has avidity for ephrin type-A receptor 2 (EphA2), wherein the peptide competes for binding to human EphA2 with a peptide that has a structure of Formula (I) as described herein (e.g., Formula (I-1) and Formula (I-2), or a pharmaceutically acceptable salt thereof.

In some embodiments, the peptide competes for binding to human EphA2 at one or more amino acid residues selected from Asp53, Met55, Asn57, Met59, Met66, Thr101, Arg103, Phe156, Glu157, Arg159, Val161, Val189, and Ala190. In some embodiments, the peptide competes for binding to human EphA2 at one or more amino acid residues selected from Asp53, Phe156, and Glu157. In some embodiments, the peptide competes for binding to human EphA2 at Asp53, Glu157, or both.

The structures of exemplary unnatural amino acids that are present in Table 1 can be found in Table 3.

As described in Tables 1, 2A, 2B, 2C, or other tables, abbreviations have the following meanings:
Lower case d means D-amino acids, e.g., dF refers to d-phenylalanine;
Me refers to a methyl group, e.g., MeG represents N-Methyl-Glycine;
Ala or A refer to alanine;
Arg or R refer to arginine;
Asn or N refer to asparagine;
Asp or D refer to aspartic acid;
Cys or C refer to cysteine;
Gln or Q refer to glutamine;
Gly or G refer to glycine;
His or H refer to histidine;
Ile or I refer to isoleucine;
Leu or L refer to leucine;
Lys or K refer to lysine;
Met or M refer to methionine;
Phe or F refer to phenylalanine;
Pro or P refer to proline;
Ser or S refer to serine;
Thr or T refer to threonine;
Trp or W refer to tryptophan;
Tyr or Y refer to tyrosine;
Val or V refer to valine;
Ahp refers to 2-aminoheptanoic acid;
Nal1 refers to 1-naphthylalanine;
Chg refers to cyclohexylglycine;
F3C refers to 3-chlorophenylalanine;
mBph refers to 3-phenylphenylalanine;
Cba refers to cyclobutylalanine;
Hph refers to homophenylalanine;
W6C refers to 6-chlorotryptaphan;
Har refers to homoarginine (i.e., hArg).

TABLE 1
Exemplary peptides sequences with avidity to EphA2

| | SEQ | Peptide Sequence | | | | | | | | | | | | | | |
|---|---|---|---|---|---|---|---|---|---|---|---|---|---|---|---|---|
| Peptide | ID | (from residue position 1 to residue position 14, if present) | | | | | | | | | | | | | | |
| Sequence of: | NO: | 1 | 2 | 3 | 4 | 5 | 6 | 7 | 8 | 9 | 10 | 11 | 12 | 13 | 14 | Term |
| PDC_EphA2- | 1 | da | MeF | N | L | Hgl | MeF | W1Me | V | W1Me | T | E | C | | | —NH₂ |
| 00001417 | | | | | | | | | | | | | | | | |
| PDC_EphA2- | 183 | da | MeF | N | L | Hgl | MeF | W1Me | V | W1Me | T | E | C | bA | dkAc | —NH₂ |
| 00008082 | | | | | | | | | | | | | | | | |
| PDC_EphA2- | 184 | da | Me3Py | N | L | Hgl | MeF | W1Me | V | W1Me | T | E | C | bA | dkAc | —NH₂ |
| 00008083 | | | | | | | | | | | | | | | | |
| PDC_EphA2- | 185 | dkCOpipzaa | Me3Py | N | L | Hgl | MeF | W1Me | V | W1Me | T | E | C | bA | dkAc | —NH₂ |
| 00008084 | | | | | | | | | | | | | | | | |
| PDC_EphA2- | 186 | dkCOpipzaa | Me3Py | N | L | KCOpipzaa | MeF | W1Me | V | W1Me | T | E | C | bA | dkAc | —NH₂ |
| 00008085 | | | | | | | | | | | | | | | | |
| PDC_EphA2- | 187 | dkCOpipzaa | Me3Py | N | L | KCOpipzaa | MeF | W1Me | V | W1Me | T | Hgn | C | bA | dkAc | —NH₂ |
| 00008086 | | | | | | | | | | | | | | | | |
| PDC_EphA2- | 188 | da | Me3Py | N | L | Hgl | MeF | W1Me | KCOpipzaa | W1Me | T | E | C | bA | dkAc | —NH₂ |
| 00008087 | | | | | | | | | | | | | | | | |
| PDC_EphA2- | 189 | da | Me3Py | N | L | KCOpipzaa | MeF | W1Me | KCOpipzaa | W1Me | T | E | C | bA | dkAc | —NH₂ |
| 00008088 | | | | | | | | | | | | | | | | |
| PDC_EphA2- | 190 | da | Me3Py | N | L | KCOpipzaa | MeF | W1Me | KCOpipzaa | W1Me | T | E | C | KAc | | —NH₂ |
| 00008089 | | | | | | | | | | | | | | | | |
| PDC_EphA2- | 191 | da | Me3Py | N | L | KCOpipzaa | MeF | W1Me | KCOpipzaa | W1Me | T | E | C | dkAc | | —NH₂ |
| 00008090 | | | | | | | | | | | | | | | | |
| PDC_EphA2- | 192 | da | Me3Py | N | L | Hgl | MeF | W1Me | KCOpipzaa | W1Me | T | Hgn | C | bA | dkAc | —NH₂ |
| 00008091 | | | | | | | | | | | | | | | | |
| PDC_EphA2- | 193 | da | Me3Py | N | L | KCOpipzaa | MeF | W1Me | KCOpipzaa | W1Me | T | Hgn | C | bA | dkAc | —NH₂ |
| 00008092 | | | | | | | | | | | | | | | | |
| PDC_EphA2- | 194 | da | Me3Py | N | L | Hgn | MeF | W1Me | KCOpipzaa | W1Me | T | Hgn | C | bA | dkAc | —NH₂ |
| 00008093 | | | | | | | | | | | | | | | | |
| PDC_EphA2- | 195 | da | Me3Py | N | L | Hgl | MeE | W1Me | KCOpipzaa | W1Me | T | Hgn | C | bA | dkAc | —NH₂ |
| 00008094 | | | | | | | | | | | | | | | | |
| PDC_EphA2- | 196 | da | Me3Py | N | L | KCOpipzaa | MeE | W1Me | KCOpipzaa | W1Me | T | Hgn | C | bA | dkAc | —NH₂ |
| 00008095 | | | | | | | | | | | | | | | | |
| PDC_EphA2- | 197 | da | Me3Py | N | L | Hgn | MeE | W1Me | KCOpipzaa | W1Me | T | Hgn | C | bA | dkAc | —NH₂ |
| 00007196 | | | | | | | | | | | | | | | | |
| PDC_EphA2- | 200 | da | Me3Py | N | L | Hgn | MeE | W1Me | KCOpipzaa | W1Me | T | Hgn | C | dk | | —NH₂ |
| 00007196- | | | | | | | | | | | | | | | | |
| C002 | | | | | | | | | | | | | | | | |
| PDC_EphA2- | 198 | A | Me3Py | N | L | Hgl | MeF | W1Me | KCOpipzaa | W1Me | T | E | C | bA | dkAc | —NH₂ |
| 00008097 | | | | | | | | | | | | | | | | |
| PDC_EphA2- | 2 | da | Me3Py | N | L | Hgn | MeF | W1Me | KCOpipzaa | W1Me | T | Hgn | C | | | —NH₂ |
| 00009998 | | | | | | | | | | | | | | | | |
| PDC_EphA2- | 3 | da | Me3Py | N | Cbg | Hgn | MeF | W1Me | KCOpipzaa | W1Me | T | Hgn | C | | | —NH₂ |
| 00009999 | | | | | | | | | | | | | | | | |
| PDC_EphA2- | 4 | da | Me3Py | N | Cbg | Hgn | N | W1Me | KCOpipzaa | W1Me | T | Hgn | C | | | —NH₂ |
| 00010000 | | | | | | | | | | | | | | | | |
| PDC_EphA2- | 5 | da | Me3Py | N | Cbg | Hgn | MeN | W1Me | KCOpipzaa | W1Me | T | Hgn | C | | | —NH₂ |
| 00010001 | | | | | | | | | | | | | | | | |
| PDC_EphA2- | 6 | da | Me3Py | N | Cbg | Hgn | MeF | W1Me7N | KCOpipzaa | W1Me | T | Hgn | C | | | —NH₂ |
| 00010002 | | | | | | | | | | | | | | | | |
| PDC_EphA2- | 7 | da | Me3Py | N | Cbg | Hgn | MeF | W1Me | KCOpipzaa | F23dMe | T | Hgn | C | | | —NH₂ |
| 00010003 | | | | | | | | | | | | | | | | |
| PDC_EphA2- | 8 | da | Me3Py | N | Cbg | Hgn | MeF | W1Me | KCOpipzaa | W1Me | T | N | C | | | —NH₂ |
| 00010004 | | | | | | | | | | | | | | | | |
| PDC_EphA2- | 9 | da | Me3Py | N | Chg | Hgn | MeF | W1Me | KCOpipzaa | W1Me | T | Hgn | C | | | —NH₂ |
| 00010005 | | | | | | | | | | | | | | | | |
| PDC_EphA2- | 10 | da | Me3Py | N | L | Hgl | MeE | W1Me | KCOpipzaa | W1Me | T | Hgn | C | | | —NH₂ |
| 00010006 | | | | | | | | | | | | | | | | |
| PDC_EphA2- | 11 | da | Me3Py | N | Cbg | Hgl | MeE | W1Me | KCOpipzaa | W1Me | T | Hgn | C | | | —NH₂ |
| 00010007 | | | | | | | | | | | | | | | | |
| PDC_EphA2- | 12 | da | Me3Py | N | L | Hgn | MeE | W1Me | KCOpipzaa | W1Me | T | Hgn | C | | | —NH₂ |
| 00010008 | | | | | | | | | | | | | | | | |
| PDC_EphA2- | 13 | da | Me3Py | N | Cbg | Hgn | MeE | W1Me | KCOpipzaa | W1Me | T | Hgn | C | | | —NH₂ |
| 00010009 | | | | | | | | | | | | | | | | |
| PDC_EphA2- | 14 | da | Me3Py | N | Cbg | Hgn | N | W1Me | V | W1Me | T | Hgn | C | | | —NH₂ |
| 00010010 | | | | | | | | | | | | | | | | |
| PDC_EphA2- | 15 | da | Me3Py | N | Cbg | Hgn | MeN | W1Me | V | W1Me | T | Hgn | C | | | —NH₂ |
| 00010011 | | | | | | | | | | | | | | | | |
| PDC_EphA2- | 16 | da | Me3Py | N | Cbg | Hgn | Me3Py | W1Me | V | W1Me | T | Hgn | C | | | —NH₂ |
| 00010012 | | | | | | | | | | | | | | | | |
| PDC_EphA2- | 17 | da | MeF | N | Cbg | Hgn | MeN | W1Me | V | W1Me | T | Hgn | C | | | —NH₂ |
| 00013693 | | | | | | | | | | | | | | | | |
| PDC_EphA2- | 180 | da | Me3Py | N | Cbg | Hgn | MeN | W1Me | V | W1Me | T | Hgn | C | bA | dk | —NH₂ |
| 00010011- | | | | | | | | | | | | | | | | |
| C003 | | | | | | | | | | | | | | | | |
| PDC_EphA2- | 181 | da | Me3Py | N | Cbg | Hgn | Me3Py | W1Me | V | W1Me | T | Hgn | C | bA | dk | —NH₂ |
| 00010012- | | | | | | | | | | | | | | | | |
| C003 | | | | | | | | | | | | | | | | |
| PDC_EphA2- | 182 | da | MeF | N | Cbg | Hgn | MeN | W1Me | V | W1Me | T | Hgn | C | bA | dk | —NH₂ |
| 00013693- | | | | | | | | | | | | | | | | |
| C003 | | | | | | | | | | | | | | | | |
| PDC_EphA2- | 18 | da | MeF | N | Cbg | Hgn | MeN | W1Me | Qglucamine | W1Me | T | Hgn | C | | | —NH₂ |
| 00013703 | | | | | | | | | | | | | | | | |
| PDC_EphA2- | 19 | da | MeF | N | Cbg | Hgn | Me3Py | W1Me | Qglucamine | W1Me | T | Hgn | C | | | —NH₂ |
| 00013704 | | | | | | | | | | | | | | | | |
| PDC_EphA2- | 20 | da | Me3Py | N | Cbg | Hgn | Me3Py | W1Me | Qglucamine | W1Me | T | Hgn | C | | | —NH₂ |
| 00013705 | | | | | | | | | | | | | | | | |
| PDC_EphA2- | 199 | da | Me3Py | N | L | Hgn | MeF | W1Me | KCOpipzaa | W1Me | T | Hgn | C | dk | | —NH₂ |
| 00008093- | | | | | | | | | | | | | | | | |
| C002 | | | | | | | | | | | | | | | | |
| PDC EphA2 | 200 | da | Me3Py | N | L | Hgn | MeE | W1Me | KCOpipzaa | W1Me | T | Hgn | C | dk | | —NH₂ |
| 00007196- | | | | | | | | | | | | | | | | |
| C002 | | | | | | | | | | | | | | | | |
| PDC_EphA2- | 201 | da | Me3Py | N | Cbg | Hgn | MeN | W1Me | V | W1Me | T | Hgn | C | dk | | —NH₂ |
| 00010011- | | | | | | | | | | | | | | | | |
| C002 | | | | | | | | | | | | | | | | |
| PDC_EphA2- | 202 | da | Me3Py | N | Cbg | Hgn | Me3Py | W1Me | V | W1Me | T | Hgn | C | dk | | —NH₂ |
| 00010012- | | | | | | | | | | | | | | | | |
| C002 | | | | | | | | | | | | | | | | |
| PDC_EphA2- | 203 | da | MeF | N | Cbg | Hgn | MeN | W1Me | V | W1Me | T | Hgn | C | dk | | —NH₂ |
| 00013693- | | | | | | | | | | | | | | | | |
| C002 | | | | | | | | | | | | | | | | |
| PDC_EphA2- | 21 | da | MeF | N | Cbg | Hgn | MeF | W1Me | Qglucamine | W1Me | T | Hgn | C | | | —NH₂ |
| 00013917 | | | | | | | | | | | | | | | | |
| PDC_EphA2- | 22 | dahp | Me3Py | N | L | Hgl | MeF | W1Me | KCOpipzaa | W1Me | T | Hgn | C | | | —NH₂ |
| 00018526 | | | | | | | | | | | | | | | | |
| PDC_EphA2- | 23 | da | Me3Py | N | L | Hgl | MeF4C | W1Me | KCOpipzaa | W1Me | T | Hgn | C | | | —NH₂ |
| 00018527 | | | | | | | | | | | | | | | | |
| PDC_EphA2- | 24 | da | Me3Py | N | L | Hgl | MeF | W1Me | KCOpipzaa | W1Me | T | Nmm | C | | | —NH₂ |
| 00018528 | | | | | | | | | | | | | | | | |
| PDC_EphA2- | 25 | da | Me3Py | N | L | Hgl | MeF | W1Me | KCOpipzaa | W1Me | T | Ndm | C | | | —NH₂ |
| 00018529 | | | | | | | | | | | | | | | | |
| PDC_EphA2- | 26 | dahp | Me3Py | N | L | Hgn | MeE | W1Me | KCOpipzaa | W1Me | T | Hgn | C | | | —NH₂ |
| 00018530 | | | | | | | | | | | | | | | | |
| PDC_EphA2- | 27 | da | Me3Py | N | L | Nmm | MeE | W1Me | KCOpipzaa | W1Me | T | Hgn | C | | | —NH₂ |
| 00018531 | | | | | | | | | | | | | | | | |
| PDC_EphA2- | 28 | da | Me3Py | N | L | Ndm | MeE | W1Me | KCOpipzaa | W1Me | T | Hgn | C | | | —NH₂ |
| 00018532 | | | | | | | | | | | | | | | | |
| PDC_EphA2- | 29 | da | Me3Py | N | L | Hgn | MeE | W1Me | KCOpipzaa | W1Me | T | Nmm | C | | | —NH₂ |
| 00018533 | | | | | | | | | | | | | | | | |
| PDC_EphA2- | 30 | da | Me3Py | N | L | Hgn | MeE | W1Me | KCOpipzaa | W1Me | T | Ndm | C | | | —NH₂ |
| 00018534 | | | | | | | | | | | | | | | | |
| PDC_EphA2- | 31 | dahp | Me3Py | N | Chg | Hgn | MeF | W1Me | KCOpipzaa | W1Me | T | Hgn | C | | | —NH₂ |
| 00018535 | | | | | | | | | | | | | | | | |
| PDC_EphA2- | 32 | da | Me3Py | N | Chg | Nmm | MeF | W1Me | KCOpipzaa | W1Me | T | Hgn | C | | | —NH₂ |
| 00018536 | | | | | | | | | | | | | | | | |
| PDC_EphA2- | 33 | da | Me3Py | N | Chg | Ndm | MeF | W1Me | KCOpipzaa | W1Me | T | Hgn | C | | | —NH₂ |
| 00018537 | | | | | | | | | | | | | | | | |
| PDC_EphA2- | 34 | da | Me3Py | N | Chg | Hgn | MeF4C | W1Me | KCOpipzaa | W1Me | T | Hgn | C | | | —NH₂ |
| 00018538 | | | | | | | | | | | | | | | | |
| PDC_EphA2- | 35 | da | Me3Py | N | Chg | Hgn | MeF | W1Me | KCOpipzaa | W1Me | T | Nmm | C | | | —NH₂ |
| 00018539 | | | | | | | | | | | | | | | | |
| PDC_EphA2- | 36 | da | Me3Py | N | Chg | Hgn | MeF | W1Me | KCOpipzaa | W1Me | T | Ndm | C | | | —NH₂ |
| 00018540 | | | | | | | | | | | | | | | | |
| PDC_EphA2- | 37 | df3CON | MeF | N | Cbg | Hgn | MeN | W1Me7C1 | V | W1Me | T | Hgn | C | | | —NH₂ |
| 00019421 | | | | | | | | | | | | | | | | |
| PDC_EphA2- | 38 | df3CON | MeF | N | L | Hgn | MeN | W1Me7C1 | V | W1Me | T | Hgn | C | | | —NH₂ |
| 00019422 | | | | | | | | | | | | | | | | |
| PDC_EphA2- | 39 | df3CON | MeF | N | L | Hgn | MeN | W1Me | V | W1Me | T | Hgn | C | | | —NH₂ |
| 00019423 | | | | | | | | | | | | | | | | |
| PDC_EphA2- | 40 | df3CON | Me3Py | N | Cbg | Hgn | MeN | W1Me7C1 | V | W1Me | T | Hgn | C | | | —NH₂ |
| 00019424 | | | | | | | | | | | | | | | | |
| PDC_EphA2- | 41 | df3CON | MeF | N | Cbg | Hgn | MeN | W1Me7C1 | KCOpipzaaa | W1Me | T | Hgn | C | | | —NH₂ |
| 00019425 | | | | | | | | | | | | | | | | |
| PDC_EphA2- | 42 | df3CON | MeF | N | Cbg | Hgn | MeN | W1Me7C1 | Hcit | W1Me | T | Hgn | C | | | —NH₂ |
| 00019426 | | | | | | | | | | | | | | | | |
| PDC_EphA2- | 43 | df3CON | MeF | N | Cbg | Hgn | MeE | W1Me7C1 | Hcit | W1Me | T | Har | C | | | —NH₂ |
| 00019427 | | | | | | | | | | | | | | | | |
| PDC_EphA2- | 44 | df3CON | Me3Py | N | Cbg | Hgn | MeN | W1Me7C1 | Hcit | W1Me | T | R | C | | | —NH₂ |
| 00019428 | | | | | | | | | | | | | | | | |
| PDC_EphA2- | 45 | df3CON | Me3Py | N | Cbg | Hgn | MeN | W1Me7C1 | Hcit | W1Me | T | Har | C | | | —NH₂ |
| 00019429 | | | | | | | | | | | | | | | | |
| PDC_EphA2- | 46 | df3CON | Me3Py | N | Cbg | Hgn | MeN | W1Me7C1 | KCOpipzaa | W1Me | T | Har | C | | | —NH₂ |
| 00019430 | | | | | | | | | | | | | | | | |
| PDC_EphA2- | 47 | df3CON | MeF | N | Cbg | Hgn | MeF | W1Me7C1 | Qglucamine | W1Me | T | Hgn | C | | | —NH₂ |
| 00019431 | | | | | | | | | | | | | | | | |
| PDC_EphA2- | 48 | df3CON | MeF | N | Cbg | Hgn | MeF | W1Me7C1 | KCOpipzaa | W1Me | T | Hgn | C | | | —NH₂ |
| 00019432 | | | | | | | | | | | | | | | | |
| PDC_EphA2- | 49 | df3CON | Me3Py | N | Cbg | Hgn | MeF | W1Me7C1 | Qglucamine | W1Me | T | Hgn | C | | | —NH₂ |
| 00019433 | | | | | | | | | | | | | | | | |
| PDC_EphA2- | 50 | df3CON | Me3Py | N | Cbg | Hgn | MeF | W1Me7C1 | KCOpipzaa | W1Me | T | Hgn | C | | | —NH₂ |
| 00019434 | | | | | | | | | | | | | | | | |
| PDC_EphA2- | 51 | df3CON | MeF | N | Cbg | Hgn | Me3Py | W1Me7C1 | Qglucamine | W1Me | T | Hgn | C | | | —NH₂ |
| 00019435 | | | | | | | | | | | | | | | | |
| PDC_EphA2- | 52 | df3CON | MeF | N | Cbg | Hgn | Me3Py | W1Me7C1 | KCOpipzaa | W1Me | T | Hgn | C | | | —NH₂ |
| 00019436 | | | | | | | | | | | | | | | | |
| PDC_EphA2- | 53 | df3CON | Me3Py | N | Cbg | Hgn | Me3Py | W1Me7C1 | Qglucamine | W1Me | T | Hgn | C | | | —NH₂ |
| 00019437 | | | | | | | | | | | | | | | | |
| PDC_EphA2- | 54 | df3CON | Me3Py | N | Cbg | Hgn | Me3Py | W1Me7C1 | KCOpipzaa | W1Me | T | Hgn | C | | | —NH₂ |
| 00019438 | | | | | | | | | | | | | | | | |
| PDC_EphA2- | 55 | df3CON | Me3Py | N | Cbg | Hgn | MeE | W1Me7C1 | Qglucamine | W1Me | T | Hgn | C | | | —NH₂ |
| 00019439 | | | | | | | | | | | | | | | | |
| PDC_EphA2- | 56 | df3CON | Me3Py | N | Cbg | Hgn | MeE | W1Me7C1 | KCOpipzaa | W1Me | T | Hgn | C | | | —NH₂ |
| 00019440 | | | | | | | | | | | | | | | | |
| PDC_EphA2- | 57 | df3CON | Me3Py | N | Cbg | KCOpipzaa | MeF | W1Me7C1 | KCOpipzaa | W1Me | T | Hgn | C | | | —NH₂ |
| 00019441 | | | | | | | | | | | | | | | | |
| PDC_EphA2- | 58 | df3CON | Me3Py | N | Cbg | KCOpipzaa | MeF | W1Me7C1 | Qglucamine | W1Me | T | Hgn | C | | | —NH₂ |
| 00019442 | | | | | | | | | | | | | | | | |
| PDC_EphA2- | 273 | df3CON | Me3Py | N | Cbg | Hgn | MeE | W1Me7C1 | KCOpipzaa | W1Me | T | hArg | C | | | —NH₂ |
| 00019443 | | | | | | | | | | | | | | | | |
| PDC_EphA2- | 204 | df3CON | Me3Py | N | Cbg | Hgn | Me3Py | W1Me7C1 | Qglucamine | W1Me | T | Hgn | C | dk | | —NH₂ |
| 00019437- | | | | | | | | | | | | | | | | |
| C002 | | | | | | | | W1Me7C1 | | | | | | | | |
| PDC_EphA2- | 205 | df3CON | Me3Py | N | Cbg | Hgn | MeN | | Qglucamine | W1Me | T | Hgn | C | dk | | —NH₂ |
| 00022593- | | | | | | | | W1Me7C1 | | | | | | | | |
| C002 | | | | | | | | | | | | | | | | |
| PDC_EphA2- | 206 | df3CON | Me3Py | N | Cbg | Qglucamine | MeN | W1Me7C1 | Qglucamine | W1Me | T | Hgn | C | dk | | —NH₂ |
| 00022594- | | | | | | | | | | | | | | | | |
| C002 | | | | | | | | | | | | | | | | |
| PDC_EphA2- | 207 | df3CON | Me3Py | N | Cbg | Hgn | MeN | W1Me | Qglucamine | W1Me | T | Hgn | C | dk | | —NH₂ |
| 00022595- | | | | | | | | | | | | | | | | |
| C002 | | | | | | | | | | | | | | | | |
| PDC_EphA2- | 150 | F | 4Py2NH2 | L | Hcit | W1Me | MeF | W1Me | C | | | | | | | —NH₂ |
| 00020708 | | | | | | | | | | | | | | | | |
| PDC_EphA2- | 246 | F | 4Py2NH2 | L | Hcit | W1Me | MeF | W1Me | C | G | | | | | | —NH₂ |
| 00022601 | | | | | | | | | | | | | | | | |
| PDC_EphA2- | 247 | F | 4Py2NH2 | L | Hcit | W1Me | MeF | W1Me | C | KAc | | | | | | —NH₂ |
| 00022602 | | | | | | | | | | | | | | | | |
| PDC_EphA2- | 248 | F | 4Py2NH2 | L | Hcit | W1Me | MeF | W1Me | C | dkAc | | | | | | —NH₂ |
| 00022603 | | | | | | | | | | | | | | | | |
| PDC_EphA2- | 123 | F | H | N | L | S | MeF | W1Me | Hcit | W1Me | C | | | | | —NH₂ |
| 00022417 | | | | | | | | | | | | | | | | |
| PDC_EphA2- | 136 | F | H | N | L | S | MeF | W1Me | Hcit | W1Me | C | G | | | | —NH₂ |
| 00022606 | | | | | | | | | | | | | | | | |
| PDC_EphA2- | 233 | F | H | N | L | S | MeF | W1Me | Hcit | W1Me | C | KAc | | | | —NH₂ |
| 00022607 | | | | | | | | | | | | | | | | |
| PDC_EphA2- | 234 | F | H | N | L | S | MeF | W1Me | Hcit | W1Me | C | dkAc | | | | —NH₂ |
| 00022608 | | | | | | | | | | | | | | | | |
| PDC_EphA2- | 208 | df3CON | Me3Py | N | Cbg | Hgn | MeE | W1Me7C1 | KCOpipzaa | W1Me | T | Hgn | C | dk | | —NH₂ |
| 00019440- | | | | | | | | | | | | | | | | |
| C002 | | | | | | | | | | | | | | | | |
| PDC_EphA2- | 209 | df3CON | MeF3CN | N | Cbg | Hgn | MeE | W1Me7C1 | KCOpipzaa | W1Me | T | Hgn | C | dk | | —NH₂ |
| 00022612- | | | | | | | | | | | | | | | | |
| C002 | | | | | | | | | | | | | | | | |
| PDC_EphA2- | 210 | df3CON | MeF3H | N | Cbg | Hgn | MeE | W1Me7C1 | KCOpipzaa | W1Me | T | Hgn | C | dk | | —NH₂ |
| 00022613- | | | | | | | | | | | | | | | | |
| C002 | | | | | | | | | | | | | | | | |
| PDC_EphA2- | 211 | df3CON | MeF3CN | N | Cbg | Hgn | Me3Py | W1Me7C1 | Qglucamine | W1Me | T | Hgn | C | dk | | —NH₂ |
| 00022614- | | | | | | | | | | | | | | | | |
| C002 | | | | | | | | | | | | | | | | |
| PDC_EphA2- | 212 | df3CON | MeF3CN | N | Cbg | Hgn | MeN | W1Me7C1 | Qglucamine | W1Me | T | Hgn | C | dk | | —NH₂ |
| 00022615- | | | | | | | | | | | | | | | | |
| C002 | | | | | | | | | | | | | | | | |
| PDC_EphA2- | 213 | da | MeF | N | Cbg | Hgn | MEN | W1Me | Qglucamine | W1Me | T | Hgn | C | dk | | —NH₂ |
| 00013703- | | | | | | | | | | | | | | | | |
| C002 | | | | | | | | | | | | | | | | |
| PDC_EphA2- | 214 | da | MeF | N | Cbg | Hgn | Me3Py | W1Me | Qglucamine | W1Me | T | Hgn | C | dk | | —NH₂ |
| 00013704- | | | | | | | | | | | | | | | | |
| C002 | | | | | | | | | | | | | | | | |
| PDC_EphA2- | 215 | da | Me3Py | N | Cbg | Hgn | Me3Py | W1Me | Qglucamine | W1Me | T | Hgn | C | dk | | —NH₂ |
| 00013705- | | | | | | | | | | | | | | | | |
| C002 | | | | | | | | | | | | | | | | |
| PDC_EphA2- | 251 | da | 4Py2NH2 | MeF | W1Me | MeF | W1Me | C | G | | | | | | | —NH₂ |
| 00019498- | | | | | | | | | | | | | | | | |
| L092 | | | | | | | | | | | | | | | | |
| PDC_EphA2- | 249 | F | 4Py | L | G | W1Me | MeF | W1Me | C | G | | | | | | —NH₂ |
| 00020295- | | | | | | | | | | | | | | | | |
| L092 | | | | | | | | | | | | | | | | |
| PDC_EphA2- | 235 | da | MeF | N | Cba | T | N | W1Me | Hcit | W1Me | C | G | | | | —NH₂ |
| 00021407- | | | | | | | | | | | | | | | | |
| L092 | | | | | | | | | | | | | | | | |
| PDC_EphA2- | 236 | F | 4Py | N | L | E | MeF | W1Me | 3Py6NH2 | W1Me | C | G | | | | —NH₂ |
| 00022057- | | | | | | | | | | | | | | | | |
| L092 | | | | | | | | | | | | | | | | |
| PDC_EphA2- | 272 | F | W1Me | W1Me | W1Me | S | 3Py6NH2 | MeF | E | C | G | | | | | —NH₂ |
| 00022062- | | | | | | | | | | | | | | | | |
| L092 | | | | | | | | | | | | | | | | |
| PDC_EphA2- | 250 | F | 4Py2NH2 | L | Hcit | W1Me | MeF | W1Me | C | dk | | | | | | —NH₂ |
| 00020708- | | | | | | | | | | | | | | | | |
| C002 | | | | | | | | | | | | | | | | |
| PDC_EphA2- | 237 | F | H | N | L | S | MeF | W1Me | Hcit | W1Me | C | dk | | | | —NH₂ |
| 00022417- | | | | | | | | | | | | | | | | |
| C002 | | | | | | | | | | | | | | | | |
| PDC_EphA2- | 59 | df3CON | Me3Py | N | L | N | Me3Py | W1Me7C1 | Qglucamine | W1Me | T | N | C | | | —NH₂ |
| 00026424 | | | | | | | | | | | | | | | | |
| PDC_EphA2- | 60 | df3CON | Me3Py | N | L | N | MeN | W1Me7C1 | Qglucamine | W1Me | T | N | C | | | —NH₂ |
| 00026425 | | | | | | | | | | | | | | | | |
| PDC_EphA2- | 61 | df3CON | Me3Py | N | L | N | MeN | W1Me7C1 | Cit | W1Me | T | N | C | | | —NH₂ |
| 00026426 | | | | | | | | | | | | | | | | |
| PDC_EphA2- | 216 | df3CON | Me3Py | N | L | N | Me3Py | W1Me7C1 | Qglucamine | W1Me | T | N | C | dk | | —NH₂ |
| 00026424- | | | | | | | | | | | | | | | | |
| C002 | | | | | | | | | | | | | | | | |
| PDC_EphA2- | 217 | df3CON | Me3Py | N | L | N | MeN | W1Me7C1 | Qglucamine | W1Me | T | N | C | dk | | —NH₂ |
| 00026425- | | | | | | | | | | | | | | | | |
| C002 | | | | | | | | | | | | | | | | |
| PDC_EphA2- | 218 | df3CON | Me3Py | N | L | N | MeN | W1Me7C1 | Cit | W1Me | T | N | C | dk | | —NH₂ |
| 00026426- | | | | | | | | | | | | | | | | |
| C002 | | | | | | | | | | | | | | | | |
| PDC_EphA2- | 151 | F | 4Py2NH2 | L | MeA | W1Me | MeF | W1Me | C | | | | | | | —NH₂ |
| 00026479 | | | | | | | | | | | | | | | | |
| PDC_EphA2- | 152 | F | 4Py2NH2 | L | MeHcit | W1Me | MeF | W1Me | C | | | | | | | —NH₂ |
| 00026481 | | | | | | | | | | | | | | | | |
| PDC EphA2 | 62 | da | Me3Py | N | L | K | MeE | W1Me7C1 | KCOpipzaa | W1Me | T | Hgn | C | | | —NH₂ |
| 00026521 | | | | | | | | | | | | | | | | |
| PDC_EphA2- | 63 | da | Me3Py | N | L | Hgn | MeE | W1Me7C1 | K | W1Me | T | Hgn | C | | | —NH₂ |
| 00026522 | | | | | | | | | | | | | | | | |
| PDC_EphA2- | 64 | da | Me3Py | N | L | Hgn | MeE | W1Me7C1 | KCOpipzaa | W1Me | T | K | C | | | —NH₂ |
| 00026523 | | | | | | | | | | | | | | | | |
| PDC_EphA2- | 65 | df3CON | Me3Py | N | L | K | MeN | W1Me7C1 | Qglucamine | W1Me | T | Hgn | C | | | —NH₂ |
| 00026524 | | | | | | | | | | | | | | | | |
| PDC_EphA2- | 66 | df3CON | Me3Py | N | L | Hgn | MeN | W1Me7C1 | K | W1Me | T | Hgn | C | | | —NH₂ |
| 00026525 | | | | | | | | | | | | | | | | |
| PDC_EphA2- | 67 | df3CON | Me3Py | N | L | Hgn | MeN | W1Me7C1 | Qglucamine | W1Me | T | K | C | | | —NH₂ |
| 00026526 | | | | | | | | | | | | | | | | |
| PDC_EphA2- | 68 | df3CON | Me3Py | N | L | K | MeE | W1Me7C1 | KCOpipzaa | W1Me | T | Hgr | C | | | —NH₂ |
| 00026527 | | | | | | | | | | | | | | | | |
| PDC_EphA2- | 69 | df3CON | Me3Py | N | L | Hgn | MeE | W1Me7C1 | K | W1Me | T | Hgr | C | | | —NH₂ |
| 00026528 | | | | | | | | | | | | | | | | |
| PDC_EphA2- | 70 | df3CON | Me3Py | N | L | Hgn | MeE | W1Me7C1 | KCOpipzaa | W1Me | T | K | C | | | —NH₂ |
| 00026529 | | | | | | | | | | | | | | | | |
| PDC_EphA2- | 124 | df3CON | Me3Py | N | L | S | MeF | W1Me7C1 | Hcit | W1Me | C | | | | | —NH₂ |
| 00026600 | | | | | | | | | | | | | | | | |
| PDC_EphA2- | 125 | df3CON | Me3Py | N | L | S | Me3Py | W1Me7C1 | Hcit | W1Me | C | | | | | —NH₂ |
| 00026601 | | | | | | | | | | | | | | | | |
| PDC_EphA2- | 126 | df3CON | Me3Py | N | Cbg | S | Me3Py | W1Me7C1 | Hcit | W1Me | C | | | | | —NH₂ |
| 00026602 | | | | | | | | | | | | | | | | |
| PDC_EphA2- | 127 | df3CON | Me3Py | N | Cbg | Qglucamine | Me3Py | W1Me7C1 | KCOpipzaa | W1Me | C | | | | | —NH₂ |
| 00026603 | | | | | | | | | | | | | | | | |
| PDC_EphA2- | 128 | df3CON | Me3Py | N | Cbg | Qglucamine | Me3Py | W1Me7C1 | Qglucamine | W1Me | C | | | | | —NH₂ |
| 00026604 | | | | | | | | | | | | | | | | |
| PDC_EphA2- | 129 | df3CON | Me4Py2NH2 | N | Cbg | Qglucamine | Me3Py | W1Me7C1 | KCOpipzaa | W1Me | C | | | | | —NH₂ |
| 00026605 | | | | | | | | | | | | | | | | |
| PDC_EphA2- | 130 | df3CON | Me3Py | N | Cbg | Hgn | MeE | W1Me7C1 | KCOpipzaa | W1Me | C | | | | | —NH₂ |
| 00026606 | | | | | | | | | | | | | | | | |
| PDC_EphA2- | 131 | df3CON | Me3Py | N | Cbg | Qglucamine | MeE | W1Me7C1 | Hcit | W1Me | C | | | | | —NH₂ |
| 00026607 | | | | | | | | | | | | | | | | |
| PDC_EphA2- | 132 | df3CON | MeF3H | N | Cbg | Hgn | MeE | W1Me7C1 | KCOpipzaa | W1Me | C | | | | | —NH₂ |
| 00026608 | | | | | | | | | | | | | | | | |
| PDC_EphA2- | 133 | df3CON | Me3Py | N | Cbg | Hgn | MeE | W1Me7C1 | KCOpipzaa | W1Me | CdMe | | | | | —NH₂ |
| 00026609 | | | | | | | | | | | | | | | | |
| PDC_EphA2- | 134 | df3CON | Me3Py | N | Cbg | Hgn | MeE | W1Me7C1 | KCOpipzaa | W1Me | C3SMe | | | | | —NH₂ |
| 00026610 | | | | | | | | | | | | | | | | |
| PDC_EphA2- | 135 | df3CON | Me3Py | N | Cbg | Hgn | MeE | W1Me7C1 | KCOpipzaa | W1Me | C3RMe | | | | | —NH₂ |
| 00026611 | | | | | | | | | | | | | | | | |
| PDC_EphA2- | 238 | df3CON | Me3Py | N | L | S | MeF | W1Me7C1 | Hcit | W1Me | C | K | | | | —NH₂ |
| 00026600- | | | | | | | | | | | | | | | | |
| C001 | | | | | | | | | | | | | | | | |
| PDC_EphA2- | 239 | df3CON | Me3Py | N | L | S | Me3Py | W1Me7C1 | Hcit | W1Me | C | K | | | | —NH₂ |
| 00026601- | | | | | | | | | | | | | | | | |
| C001 | | | | | | | | | | | | | | | | |
| PDC_EphA2- | 240 | df3CON | Me3Py | N | Cbg | S | Me3Py | W1Me7C1 | Hcit | W1Me | C | K | | | | —NH₂ |
| 00026602- | | | | | | | | | | | | | | | | |
| C001 | | | | | | | | | | | | | | | | |
| PDC_EphA2- | 241 | df3CON | Me3Py | N | Cbg | Qglucamine | Me3Py | W1Me7C1 | KCOpipzaa | W1Me | C | K | | | | —NH₂ |
| 00026603- | | | | | | | | | | | | | | | | |
| C001 | | | | | | | | | | | | | | | | |
| PDC_EphA2- | 242 | df3CON | Me3Py | N | Cbg | Qglucamine | Me3Py | W1Me7C1 | Qglucamine | W1Me | C | K | | | | —NH₂ |
| 00026604- | | | | | | | | | | | | | | | | |
| C001 | | | | | | | | | | | | | | | | |
| PDC_EphA2- | 243 | df3CON | Me3Py | N | Cbg | Hgn | MeE | W1Me7C1 | KCOpipzaa | W1Me | C | K | | | | —NH₂ |
| 00026606- | | | | | | | | | | | | | | | | |
| C001 | | | | | | | | | | | | | | | | |
| PDC_EphA2- | 244 | df3CON | Me3Py | N | Cbg | Qglucamine | MeE | W1Me7C1 | Hcit | W1Me | C | K | | | | —NH₂ |
| 00026607- | | | | | | | | | | | | | | | | |
| C001 | | | | | | | | | | | | | | | | |
| PDC_EphA2- | 245 | df3CON | MeF3H | N | Cbg | Hgn | MeE | W1Me7C1 | KCOpipzaa | W1Me | C | K | | | | —NH₂ |
| 00026608- | | | | | | | | | | | | | | | | |
| C001 | | | | | | | | | | | | | | | | |
| PDC_EphA2- | 219 | da | Me3Py | N | L | Hgn | MeE | W1Me | KCOpipzaa | W1Me | T | Hgn | C | dk | | —OH |
| 00026624 | | | | | | | | | | | | | | | | |
| PDC_EphA2- | 220 | da | MeF | N | Cbg | Hgn | MeN | W1Me | V | W1Me | T | Hgn | C | dk | | —OH |
| 00026625 | | | | | | | | | | | | | | | | |
| PDC_EphA2- | 221 | df3CON | Me3Py | N | Cbg | Hgn | Me3Py | W1Me7C1 | Qglucamine | W1Me | T | Hgn | C | dk | | —OH |
| 00026626 | | | | | | | | | | | | | | | | |
| PDC_EphA2- | 222 | df3CON | MeF3H | N | Cbg | Hgn | MeE | W1Me7C1 | KCOpipzaa | W1Me | T | Hgn | C | dk | | —OH |
| 00026627 | | | | | | | | | | | | | | | | |
| PDC_EphA2- | 254 | da | Me3Py | N | Cbg | K | MeE | W1Me7C1 | KCOpipzaa | W1Me | T | Hgn | C | de | | —OH |
| 00027090 | | | | | | | | | | | | | | | | |
| PDC_EphA2- | 255 | df3CON | Me3Py | N | Cbg | K | MeE | W1Me7C1 | KCOpipzaa | W1Me | T | Hgn | C | de | | —OH |
| 00027091 | | | | | | | | | | | | | | | | |
| PDC_EphA2- | 256 | da | Me3Py | N | Cbg | K | MeE | W1Me7C1 | KCOpipzaa | W1Me | T | Hgn | C | de | | —NH₂ |
| 00027092 | | | | | | | | | | | | | | | | |
| PDC_EphA2- | 252 | df3CON | Me3Py | N | Cbg | Hgn | MeE | W1Me7N | KCOpipzaa | W1Me | T | Hgn | C | dk | | —NH₂ |
| 00027094- | | | | | | | | | | | | | | | | |
| C002 | | | | | | | | | | | | | | | | |
| PDC_EphA2- | 253 | da | Me3Py | N | Cbg | Hgn | MeE | W1Me7N | KCOpipzaa | W1Me | T | Hgn | C | dk | | —NH₂ |
| 00027095- | | | | | | | | | | | | | | | | |
| C002 | | | | | | | | | | | | | | | | |
| PDC_EphA2- | 267 | da | Me3Py | N | L | Hgn | MeE | W1Me | KCOpipzaa | W1Me | T | hArg | C | dk | | —NH₂ |
| 00008010- | | | | | | | | | | | | | | | | |
| C002 | | | | | | | | | | | | | | | | |

“Term” refers to the functional group at the C-terminus.

TABLE 2A-Lu
Exemplary conjugates of the present disclosure containing a chelated cold Lutetium (Lu-175) (12mer cyclic peptides).
“Term” refers to the functional group at the C-terminus.

| | | Conjugate structure (including a monocyclic peptide and a metal chelator, e.g., | | | | | | | | |
|---|---|---|---|---|---|---|---|---|---|---|
| | | DOTA; in the monocyclic peptide, the amino acid residue at position 1 and the cysteine | | | | | | | | |
| | | residue at position 12 are covalently connected through —C(═O)—CH₂— bond, with C═O group attached | | | | | | | | |
| | SEQ | to the N-terminal group of amino acid residue at position 1 and CH₂ attached to the —S— from the cysteine) | | | | | | | | |
| Conjugate | ID | From residue position 1 to residue position 12 | | | | | | | | |
| ID | NO: | 1 | 2 | 3 | 4 | 5 | 6 | 7 | 8 | 9 |
| PDC_EphA2- | 280 | da | MeF | N | L | Hgl | MeF | W1Me | V | W1Me |
| 00001418- | | | | | | | | | | |
| C306 | | | | | | | | | | |
| PDC_EphA2- | 281 | da | MeF | N | L | Hgl | MeF | W1Me | V | W1Me |
| 00001419- | | | | | | | | | | |
| C30x | | | | | | | | | | |
| PDC_EphA2- | 282 | da | MeF | N | L | Hgl | MeF | W1Me | V | W1Me |
| 00001417- | | | | | | | | | | |
| C306 | | | | | | | | | | |
| PDC_EphA2- | 283 | da | MeF | N | L | Hgl | MeF | W1Me | V | W1Me |
| 00001417- | | | | | | | | | | |
| C304 | | | | | | | | | | |
| PDC_EphA2- | 284 | da | MeF | N | L | Hgl | MeF | W1Me | V | W1Me |
| 00001417- | | | | | | | | | | |
| C302 | | | | | | | | | | |
| PDC_EphA2- | 285 | da | MeF | N | L | Hgl | MeF | W1Me | V | W1Me |
| 00001420- | | | | | | | | | | |
| C30x | | | | | | | | | | |
| PDC_EphA2- | 286 | da | Me3Py | N | L | Hgn | MeF | W1Me | KCOpipzaa | W1Me |
| 00008093- | | | | | | | | | | |
| C310 | | | | | | | | | | |
| PDC_EphA2- | 287 | da | Me3Py | N | L | Hgn | MeF | W1Me | KCOpipzaa | W1Me |
| 00008093- | | | | | | | | | | |
| C304 | | | | | | | | | | |
| PDC_EphA2- | 288 | da | Me3Py | N | L | Hgn | MeF | W1Me | KCOpipzaa | W1Me |
| 00008093- | | | | | | | | | | |
| C302 | | | | | | | | | | |
| PDC_EphA2- | 289 | da | Me3Py | N | L | Hgl | MeE | W1Me | KCOpipzaa | W1Me |
| 00008094- | | | | | | | | | | |
| C304 | | | | | | | | | | |
| PDC_EphA2- | 290 | da | Me3Py | N | L | Hgn | MeE | W1Me | KCOpipzaa | W1Me |
| 00007196- | | | | | | | | | | |
| C304 | | | | | | | | | | |
| PDC_EphA2- | 291 | da | Me3Py | N | L | Hgn | MeE | W1Me | KCOpipzaa | W1Me |
| 00007196- | | | | | | | | | | |
| C310 | | | | | | | | | | |
| PDC_EphA2- | 292 | da | Me3Py | N | L | Hgn | MeE | W1Me | KCOpipzaa | W1Me |
| 00007196- | | | | | | | | | | |
| C302 | | | | | | | | | | |
| PDC_EphA2- | 293 | da | Me3Py | N | L | Hgn | MeE | W1Me | KCOpipzaa | W1Me |
| 00007196- | | | | | | | | | | |
| C312 | | | | | | | | | | |
| PDC_EphA2- | 294 | da | Me3Py | N | L | Hgn | MeE | W1Me | KCOpipzaa | W1Me |
| 00007196- | | | | | | | | | | |
| C342 | | | | | | | | | | |
| PDC_EphA2- | 295 | da | Me3Py | N | L | Hgn | MeE | W1Me | KCOpipzaa | W1Me |
| 00007196- | | | | | | | | | | |
| C332 | | | | | | | | | | |
| PDC_EphA2- | 296 | da | Me3Py | N | L | Hgn | MeE | W1Me | KCOpipzaa | W1Me |
| 00007196- | | | | | | | | | | |
| C322 | | | | | | | | | | |
| PDC_EphA2- | 297 | da | Me3Py | N | L | Hgn | MeE | W1Me | KCOpipzaa | W1Me |
| 00007196- | | | | | | | | | | |
| C305 | | | | | | | | | | |
| PDC_EphA2- | 298 | da | Me3Py | N | L | Hgn | MeE | W1Me | KCOpipzaa | W1Me |
| 00026772 | | | | | | | | | | |
| PDC_EphA2- | 299 | da | Me3Py | N | L | Hgn | MeE | W1Me | KCOpipzaa | W1Me |
| 00007197- | | | | | | | | | | |
| C30x | | | | | | | | | | |
| PDC_EphA2- | 300 | da | Me3Py | N | L | Hgn | MeE | W1Me | KCOpipzaa | W1Me |
| 00008010- | | | | | | | | | | |
| C302 | | | | | | | | | | |
| PDC_EphA2- | 301 | da | Me3Py | N | L | Hgn | MeN | W1Me | KCOpipzaa | W1Me |
| 00007198- | | | | | | | | | | |
| C302 | | | | | | | | | | |
| PDC_EphA2- | 302 | da | Me3Py | N | L | Hgn | MeE | W1Me | KCOpipzaa | W1Me |
| 00007199- | | | | | | | | | | |
| C302 | | | | | | | | | | |
| PDC_EphA2- | 303 | da | Me3Py | N | L | Hgn | MeE | W1Me | KCOpipzaa | W1Me |
| 00007180- | | | | | | | | | | |
| C302 | | | | | | | | | | |
| PDC_EphA2- | 304 | da | Me3Py | N | L | Hgn | MeE | W1Me | KCOpipzaa | W1Me |
| 00007196- | | | | | | | | | | |
| C306 | | | | | | | | | | |
| PDC_EphA2- | 305 | da | Me3Py | N | L | Hgn | MeE | W1Me | KCOpipzaa | W1Me |
| 00007196- | | | | | | | | | | |
| C307 | | | | | | | | | | |
| PDC_EphA2- | 306 | da | Me3Py | N | L | Hgn | MeE | W1Me | KCOpipzaa | W1Me |
| 00007196- | | | | | | | | | | |
| C308 | | | | | | | | | | |
| PDC_EphA2- | 307 | da | Me3Py | N | L | Hgn | MeE | W1Me | KCOpipzaa | W1Me |
| 00007196- | | | | | | | | | | |
| C309 | | | | | | | | | | |
| PDC_EphA2- | 308 | da | Me3Py | N | Cbg | Hgn | MeE | W1Me7Cl | KCOpipzaa | W1Me |
| 00027131 | | | | | | | | | | |
| PDC_EphA2- | 309 | da | Me3Py | N | Cbg | Hgn | MeE | W1Me7Cl | KCOpipzaa | W1Me |
| 00027129 | | | | | | | | | | |
| PDC_EphA2- | 310 | da | Me3Py | N | Cbg | Hgn | MeE | W1Me7Cl | KCOpipzaa | W1Me |
| 00027128 | | | | | | | | | | |
| PDC_EphA2- | 311 | da | Me3Py | N | Cbg | K(tma- | MeE | W1Me7Cl | KCOpipzaa | W1Me |
| 00027130 | | | | | | DOTA | | | | |
| | | | | | | (Lu)) | | | | |
| PDC_EphA2- | 312 | da | Me3Py | N | Cbg | K(df- | MeE | W1Me7Cl | KCOpipzaa | W1Me |
| 00027127 | | | | | | DOTA | | | | |
| | | | | | | (Lu)) | | | | |
| PDC_EphA2- | 313 | da | Me3Py | N | Cbg | K(de- | MeE | W1Me7Cl | KCOpipzaa | W1Me |
| 00027126 | | | | | | DOTA | | | | |
| | | | | | | (Lu)) | | | | |
| PDC_EphA2- | 314 | da | MeF | N | Cbg | Hgn | MeN | W1Me | V | W1Me |
| 00013693- | | | | | | | | | | |
| C304 | | | | | | | | | | |
| PDC_EphA2- | 315 | da | MeF | N | Cbg | Hgn | MeN | W1Me | V | W1Me |
| 00013693- | | | | | | | | | | |
| C303 | | | | | | | | | | |
| PDC_EphA2- | 316 | da | MeF | N | Cbg | Hgn | MeN | W1Me | V | W1Me |
| 00026773 | | | | | | | | | | |
| PDC_EphA2- | 317 | da | MeF | N | Cbg | Hgn | MeN | W1Me | V | W1Me |
| 00013693- | | | | | | | | | | |
| C302 | | | | | | | | | | |
| PDC_EphA2- | 318 | da | MeF | N | Cbg | Hgn | MeN | W1Me | Qglucamine | W1Me |
| 00013703- | | | | | | | | | | |
| C302 | | | | | | | | | | |
| PDC_EphA2- | 319 | da | MeF | N | Cbg | Hgn | Me3Py | W1Me | Qglucamine | W1Me |
| 00013704- | | | | | | | | | | |
| C304 | | | | | | | | | | |
| PDC_EphA2- | 320 | da | MeF | N | Cbg | Hgn | Me3Py | W1Me | Qglucamine | W1Me |
| 00013704- | | | | | | | | | | |
| C302 | | | | | | | | | | |
| PDC_EphA2- | 321 | da | Me3Py | N | Cbg | Hgn | Me3Py | W1Me | Qglucamine | W1Me |
| 00013705- | | | | | | | | | | |
| C304 | | | | | | | | | | |
| PDC_EphA2- | 322 | da | Me3Py | N | Cbg | Hgn | Me3Py | W1Me | Qglucamine | W1Me |
| 00013705- | | | | | | | | | | |
| C302 | | | | | | | | | | |
| PDC_EphA2- | 323 | df3CON | Me3Py | N | Cbg | Hgn | Me3Py | W1Me7Cl | Qglucamine | W1Me |
| 00019437- | | | | | | | | | | |
| C302 | | | | | | | | | | |
| PDC_EphA2- | 324 | df3CON | Me3Py | N | Cbg | Hgn | Me3Py | W1Me7Cl | Qglucamine | W1Me |
| 00026774 | | | | | | | | | | |
| PDC_EphA2- | 325 | df3CON | Me3Py | N | Cbg | Hgn | MeN | W1Me7Cl | Qglucamine | W1Me |
| 00022593- | | | | | | | | | | |
| C302 | | | | | | | | | | |
| PDC_EphA2- | 326 | df3CON | Me3Py | N | Cbg | Qglucamine | MeN | W1Me7Cl | Qglucamine | W1Me |
| 00022594- | | | | | | | | | | |
| C302 | | | | | | | | | | |
| PDC_EphA2- | 327 | df3CON | Me3Py | N | Cbg | Hgn | MeE | W1Me7Cl | Qglucamine | W1Me |
| 00019439- | | | | | | | | | | |
| C304 | | | | | | | | | | |
| PDC_EphA2- | 328 | df3CON | Me3Py | N | Cbg | Hgn | MeE | W1Me7Cl | Qglucamine | W1Me |
| 00019439- | | | | | | | | | | |
| C302 | | | | | | | | | | |
| PDC_EphA2- | 329 | df3CON | Me3Py | N | Cbg | Hgn | MeE | W1Me7Cl | KCOpipzaa | W1Me |
| 00019440- | | | | | | | | | | |
| C302 | | | | | | | | | | |
| PDC_EphA2- | 330 | df3CON | Me3Py | N | Cbg | Hgn | MeE | W1Me7Cl | KCOpipzaa | W1Me |
| 00019443- | | | | | | | | | | |
| C302 | | | | | | | | | | |
| PDC_EphA2- | 331 | df3CON | Me3Py | N | Cbg | Hgn | MeE | W1Me7Cl | KCOpipzaa | W1Me |
| 00019444- | | | | | | | | | | |
| C302 | | | | | | | | | | |
| PDC_EphA2- | 332 | df3CON | MeF3H | N | Cbg | Hgn | MeE | W1Me7Cl | KCOpipzaa | W1Me |
| 00022613- | | | | | | | | | | |
| C302 | | | | | | | | | | |
| PDC_EphA2- | 333 | df3CON | MeF3H | N | Cbg | Hgn | MeE | W1Me7Cl | KCOpipzaa | W1Me |
| 00026775 | | | | | | | | | | |
| PDC_EphA2- | 334 | da | Me3Py | N | L | K(DOTA- | MeE | W1Me7Cl | KCOpipzaa | W1Me |
| 00026631 | | | | | | Lu) | | | | |
| PDC_EphA2- | 335 | df3CON | Me3Py | N | L | K(DOTA- | MeN | W1Me7Cl | Qglucamine | W1Me |
| 00026714 | | | | | | Lu) | | | | |
| PDC_EphA2- | 336 | df3CON | Me3Py | N | L | K(DOTA- | MeE | W1Me7Cl | KCOpipzaa | W1Me |
| 00026717 | | | | | | Lu) | | | | |
| PDC_EphA2- | 337 | da | Me3Py | N | L | Hgn | MeE | W1Me7Cl | K(DOTA- | W1Me |
| 00026632 | | | | | | | | | Lu) | |
| PDC_EphA2- | 338 | df3CON | Me3Py | N | L | Hgn | MeN | W1Me7Cl | K(DOTA- | W1Me |
| 00026715 | | | | | | | | | Lu) | |
| PDC_EphA2- | 339 | df3CON | Me3Py | N | L | Hgn | MeE | W1Me7Cl | K(DOTA- | W1Me |
| 00026718 | | | | | | | | | Lu) | |
| PDC_EphA2- | 340 | da | Me3Py | N | L | Hgn | MeE | W1Me7Cl | KCOpipzaa | W1Me |
| 00026633 | | | | | | | | | | |
| PDC_EphA2- | 341 | df3CON | Me3Py | N | L | Hgn | MeN | W1Me7Cl | Qglucamine | W1Me |
| 00026716 | | | | | | | | | | |
| PDC_EphA2- | 342 | df3CON | Me3Py | N | L | Hgn | MeE | W1Me7Cl | KCOpipzaa | W1Me |
| 00026719 | | | | | | | | | | |
| PDC_EphA2- | 343 | da | Me3Py | N | Cbg | Hgn | MeN | W1Me | V | W1Me |
| 00010011- | | | | | | | | | | |
| C302 | | | | | | | | | | |
| PDC_EphA2- | 344 | da | Me3Py | N | Cbg | Hgn | MeN | W1Me | V | W1Me |
| 00010011- | | | | | | | | | | |
| C304 | | | | | | | | | | |
| PDC_EphA2- | 345 | da | Me3Py | N | Cbg | Hgn | Me3Py | W1Me | V | W1Me |
| 00010012- | | | | | | | | | | |
| C302 | | | | | | | | | | |
| PDC_EphA2- | 346 | da | Me3Py | N | Cbg | Hgn | Me3Py | W1Me | V | W1Me |
| 00010012- | | | | | | | | | | |
| C304 | | | | | | | | | | |
| PDC_EphA2- | 347 | df3CON | Me3Py | N | Cbg | Hgn | MeN | W1Me | Qglucamine | W1Me |
| 00022595- | | | | | | | | | | |
| C302 | | | | | | | | | | |
| PDC_EphA2- | 348 | df3CON | MeF3CN | N | Cbg | Hgn | MeE | W1Me7C | KCOpipzaa | W1Me |
| 00022612- | | | | | | | | | | |
| C302 | | | | | | | | | | |
| PDC_EphA2- | 349 | df3CON | MeF3CN | N | Cbg | Hgn | Me3Py | W1Me7C | Qglucamine | W1Me |
| 00022614- | | | | | | | | | | |
| C302 | | | | | | | | | | |
| PDC_EphA2- | 350 | df3CON | MeF3CN | N | Cbg | Hgn | MeN | W1Me7C | Qglucamine | W1Me |
| 00022615- | | | | | | | | | | |
| C302 | | | | | | | | | | |
| PDC_EphA2- | 351 | df3CON | Me3Py | N | L | N | Me3Py | W1Me7C | Qglucamine | W1Me |
| 00026424- | | | | | | | | | | |
| C302 | | | | | | | | | | |
| PDC_EphA2- | 352 | df3CON | Me3Py | N | L | N | MeN | W1Me7C | Qglucamine | W1Me |
| 00026425- | | | | | | | | | | |
| C302 | | | | | | | | | | |
| PDC_EphA2- | 353 | df3CON | Me3Py | N | | N | MeN | W1Me7Cl | Cit | W1Me |
| 00026426- | | | | | | | | | | |
| C302 | | | | | | | | | | |
| PDC_EphA2- | 354 | da | Me3Py | N | Cbg | Hgn | MeN | W1Me | V | W1Me |
| 00010011- | | | | | | | | | | |
| C303 | | | | | | | | | | |
| PDC_EphA2- | 355 | da | Me3Py | N | Cbg | Hgn | Me3Py | W1Me | V | W1Me |
| 00010012- | | | | | | | | | | |
| C303 | | | | | | | | | | |
| PDC_EphA2- | 356 | df3CON | Me3Py | N | Cbg | K(DOTA(Lu)) | MeE | W1Me7Cl | KCOpipzaa | W1Me |
| 00027104 | | | | | | | | | | |
| PDC_EphA2- | 357 | df3CON | Me3Py | N | Cbg | K(DOTA(Lu)) | MeE | W1Me7Cl | KCOpipzaa | W1Me |
| 00027106 | | | | | | | | | | |
| PDC_EphA2- | 358 | da | Me3Py | N | Cbg | K(DOTA(Lu)) | MeE | W1Me7Cl | KCOpipzaa | W1Me |
| 00027103 | | | | | | | | | | |
| PDC_EphA2- | 359 | da | Me3Py | N | Cbg | K(DOTA(Lu)) | MeE | W1Me7Cl | KCOpipzaa | W1Me |
| 00027105 | | | | | | | | | | |
| PDC_EphA2- | 360 | df3CON | Me3Py | N | L | Hgn | MeE | W1Me7Cl | K(DOTA(Lu)) | W1Me |
| 00026528- | | | | | | | | | | |
| 301 | | | | | | | | | | |
| PDC_EphA2- | 361 | df3CON | Me3Py | N | L | Hgn | MeE | W1Me7Cl | KCOpipzaa | W1Me |
| 00026529- | | | | | | | | | | |
| 301 | | | | | | | | | | |

Exemplary conjugates of the present disclosure containing a chelated cold Lutetium (Lu-175) (12mer cyclic peptides).
“Term” refers to the functional group at the C-terminus.

| | | Conjugate structure (including a monocyclic peptide and a metal chelator, e.g., | | | | | |
|---|---|---|---|---|---|---|---|
| | | DOTA; in the monocyclic peptide, the amino acid residue at position 1 and the cysteine | | | | | |
| | | residue at position 12 are covalently connected through —C(═O)—CH₂— bond, with C═O group attached | | | | | |
| | SEQ | to the N-terminal group of amino acid residue at position 1 and CH₂ attached to the —S— from the cysteine) | | | | | |
| Conjugate | ID | From residue position 1 to residue position 12 | | | | | |
| ID | NO: | 10 | 11 | 12 | 13 | 14 | Term |
| PDC_EphA2- | 280 | T | E | C | Sar | dk(aeea- | —NH₂ |
| 00001418- | | | | | | PEG8- | |
| C306 | | | | | | DOTA(Lu)) | |
| PDC_EphA2- | 281 | T | E | C | Dab | PEG3- | —NH₂ |
| 00001419- | | | | | | DOTA(Lu) | |
| C30x | | | | | | | |
| PDC_EphA2- | 282 | T | E | C | bA | dk(aeea- | —NH₂ |
| 00001417- | | | | | | PEG8- | |
| C306 | | | | | | DOTA(Lu)) | |
| PDC_EphA2- | 283 | T | E | C | bA | dk(PEG2- | —NH₂ |
| 00001417- | | | | | | PEG8- | |
| C304 | | | | | | DOTA(Lu)) | |
| PDC_EphA2- | 284 | T | E | C | dk(DOTA(Lu)) | | —NH₂ |
| 00001417- | | | | | | | |
| C302 | | | | | | | |
| PDC_EphA2- | 285 | T | E | C | bA | dk(PEG2- | —NH₂ |
| 00001420- | | | | | | dk(DOTA(Lu))- | |
| C30x | | | | | | PEG8-Ac) | |
| PDC_EphA2- | 286 | T | Hgn | C | dk(DOTA- | | —NH₂ |
| 00008093- | | | | | Lu-bA- | | |
| C310 | | | | | MeG-MeG- | | |
| | | | | | MeG-MeG- | | |
| | | | | | MeG-MeG- | | |
| | | | | | MeG-MeG- | | |
| | | | | | MeG- | | |
| | | | | | MeG(SEQ | | |
| | | | | | ID NO: | | |
| | | | | | 279)) | | |
| PDC_EphA2- | 287 | T | Hgn | C | bA | dk(PEG2- | —NH₂ |
| 00008093- | | | | | | PEG8- | |
| C304 | | | | | | DOTA(Lu)) | |
| PDC_EphA2- | 288 | T | Hgn | C | dk(DOTA(Lu)) | | —NH₂ |
| 00008093- | | | | | | | |
| C302 | | | | | | | |
| PDC_EphA2- | 289 | T | Hgn | C | bA | dk(PEG2- | —NH₂ |
| 00008094- | | | | | | PEG8- | |
| C304 | | | | | | DOTA(Lu)) | |
| PDC_EphA2- | 290 | T | Hgn | C | bA | dk(PEG2- | —NH₂ |
| 00007196- | | | | | | PEG8- | |
| C304 | | | | | | DOTA(Lu)) | |
| PDC_EphA2- | 291 | T | Hgn | C | dk(DOTA- | | —NH₂ |
| 00007196- | | | | | Lu-bA- | | |
| C310 | | | | | MeG-MeG- | | |
| | | | | | MeG-MeG- | | |
| | | | | | MeG-MeG- | | |
| | | | | | MeG-MeG- | | |
| | | | | | MeG- | | |
| | | | | | MeG(SEQ | | |
| | | | | | ID NO: | | |
| | | | | | 279)) | | |
| PDC_EphA2- | 292 | T | Hgn | C | dk(DOTA(Lu)) | | —NH₂ |
| 00007196- | | | | | | | |
| C302 | | | | | | | |
| PDC_EphA2- | 293 | T | Hgn | C | dk(DOTAG | | —NH₂ |
| 00007196- | | | | | A(Lu)) | | |
| C312 | | | | | | | |
| PDC_EphA2- | 294 | T | Hgn | C | dk(tetra-Et- | | —NH₂ |
| 00007196- | | | | | DOTA(Lu)) | | |
| C342 | | | | | | | |
| PDC_EphA2- | 295 | T | Hgn | C | dk(PCTA(Lu)) | | —NH₂ |
| 00007196- | | | | | | | |
| C332 | | | | | | | |
| PDC_EphA2- | 296 | T | Hgn | C | dk(p-SCN- | | —NH₂ |
| 00007196- | | | | | Bn- | | |
| C322 | | | | | PCTA(Lu)) | | |
| PDC_EphA2- | 297 | T | Hgn | C | dk-Traut- | | —NH₂ |
| 00007196- | | | | | (maleimide- | | |
| C305 | | | | | nBu- | | |
| | | | | | DOTA(Lu)) | | |
| PDC_EphA2- | 298 | T | Hgn | C | dk(DOTA(Lu)) | | —NH₂ |
| 00026772 | | | | | | | |
| PDC_EphA2- | 299 | T | Hgn | C | Medk(DOT | | —NH₂ |
| 00007197- | | | | | A(Lu)) | | |
| C30x | | | | | | | |
| PDC_EphA2- | 300 | T | hArg | C | dk(DOTA(Lu)) | | —NH₂ |
| 00008010- | | | | | | | |
| C302 | | | | | | | |
| PDC_EphA2- | 301 | T | hArg | C | dk(DOTA(Lu)) | | —NH₂ |
| 00007198- | | | | | | | |
| C302 | | | | | | | |
| PDC_EphA2- | 302 | T | E | C | dk(DOTA(Lu)) | | —NH₂ |
| 00007199- | | | | | | | |
| C302 | | | | | | | |
| PDC_EphA2- | 303 | T | hCit | C | dk(DOTA(Lu)) | | —NH₂ |
| 00007180- | | | | | | | |
| C302 | | | | | | | |
| PDC_EphA2- | 304 | T | Hgn | C- | — | | |
| 00007196- | | | | hexa- | | | |
| C306 | | | | methy- | | | |
| | | | | lenedi- | | | |
| | | | | amine-df- | | | |
| | | | | DOTA(Lu) | | | |
| PDC_EphA2- | 305 | T | Hgn | C- | — | | |
| 00007196- | | | | hexa- | | | |
| C307 | | | | methy- | | | |
| | | | | lenedi- | | | |
| | | | | amine-df- | | | |
| | | | | DOTA(Lu) | | | |
| PDC_EphA2- | 306 | T | Hgn | C- | | | —NH₂ |
| 00007196- | | | | dk(df- | | | |
| C308 | | | | DOTA(Lu)) | | | |
| PDC_EphA2- | 307 | T | Hgn | C- | | | —NH₂ |
| 00007196- | | | | dk(df- | | | |
| C309 | | | | DOTA(Lu)) | | | |
| PDC_EphA2- | 308 | T | K(tma- | C | | | —NH₂ |
| 00027131 | | | DOTA(Lu)) | | | | |
| PDC_EphA2- | 309 | T | K(df- | C | | | —NH₂ |
| 00027129 | | | DOTA(Lu)) | | | | |
| PDC_EphA2- | 310 | T | K(de- | C | | | —NH₂ |
| 00027128 | | | DOTA(Lu)) | | | | |
| PDC_EphA2- | 311 | T | Hgn | C | de | | —NH₂ |
| 00027130 | | | | | | | |
| PDC_EphA2- | 312 | T | Hgn | C | de | | —NH₂ |
| 00027127 | | | | | | | |
| PDC_EphA2- | 313 | T | Hgn | C | de | | —NH₂ |
| 00027126 | | | | | | | |
| PDC_EphA2- | 314 | T | Hgn | C | bA | dk(PEG2- | —NH₂ |
| 00013693- | | | | | | PEG8- | |
| C304 | | | | | | DOTA(Lu)) | |
| PDC_EphA2- | 315 | T | Hgn | C | bA | dk(DOTA(Lu)) | —NH₂ |
| 00013693- | | | | | | | |
| C303 | | | | | | | |
| PDC_EphA2- | 316 | T | Hgn | C | dk(DOTA(Lu)) | | —OH |
| 00026773 | | | | | | | |
| PDC_EphA2- | 317 | T | Hgn | C | dk(DOTA(Lu)) | | —NH₂ |
| 00013693- | | | | | | | |
| C302 | | | | | | | |
| PDC_EphA2- | 318 | T | Hgn | C | dk(DOTA(Lu)) | | —NH₂ |
| 00013703- | | | | | | | |
| C302 | | | | | | | |
| PDC_EphA2- | 319 | T | Hgn | C | bA | dk(PEG2- | —NH₂ |
| 00013704- | | | | | | PEG8- | |
| C304 | | | | | | DOTA(Lu)) | |
| PDC_EphA2- | 320 | T | Hgn | C | dk(DOTA(Lu)) | | —NH₂ |
| 00013704- | | | | | | | |
| C302 | | | | | | | |
| PDC_EphA2- | 321 | T | Hgn | C | bA | dk(PEG2- | —NH₂ |
| 00013705- | | | | | | PEG8- | |
| C304 | | | | | | DOTA(Lu)) | |
| PDC_EphA2- | 322 | T | Hgn | C | dk(DOTA(Lu)) | | —NH₂ |
| 00013705- | | | | | | | |
| C302 | | | | | | | |
| PDC_EphA2- | 323 | T | Hgn | C | dk(DOTA(Lu)) | | —NH₂ |
| 00019437- | | | | | | | |
| C302 | | | | | | | |
| PDC_EphA2- | 324 | T | Hgn | C | dk(DOTA(Lu)) | | —OH |
| 00026774 | | | | | | | |
| PDC_EphA2- | 325 | T | Hgn | C | dk(DOTA(Lu)) | | —NH₂ |
| 00022593- | | | | | | | |
| C302 | | | | | | | |
| PDC_EphA2- | 326 | T | Hgn | C | dk(DOTA(Lu)) | | —NH₂ |
| 00022594- | | | | | | | |
| C302 | | | | | | | |
| PDC_EphA2- | 327 | T | Hgn | C | bA | dk(PEG2- | —NH₂ |
| 00019439- | | | | | | PEG8- | |
| C304 | | | | | | DOTA(Lu)) | |
| PDC_EphA2- | 328 | T | Hgn | C | dk(DOTA(Lu)) | | —NH₂ |
| 00019439- | | | | | | | |
| C302 | | | | | | | |
| PDC_EphA2- | 329 | T | Hgn | C | dk(DOTA(Lu)) | | —NH₂ |
| 00019440- | | | | | | | |
| C302 | | | | | | | |
| PDC_EphA2- | 330 | T | hArg | C | dk(DOTA(Lu)) | | —NH₂ |
| 00019443- | | | | | | | |
| C302 | | | | | | | |
| PDC_EphA2- | 331 | T | hArg | C | dk(DOTA(Lu)) | | —OH |
| 00019444- | | | | | | | |
| C302 | | | | | | | |
| PDC_EphA2- | 332 | T | Hgr | C | dk(DOTA(Lu)) | | —NH₂ |
| 00022613- | | | | | | | |
| C302 | | | | | | | |
| PDC_EphA2- | 333 | T | Hgn | C | dk(DOTA(Lu)) | | —OH |
| 00026775 | | | | | | | |
| PDC_EphA2- | 334 | T | Hgn | C | | | —NH₂ |
| 00026631 | | | | | | | |
| PDC_EphA2- | 335 | T | Hgr | C | | | —NH₂ |
| 00026714 | | | | | | | |
| PDC_EphA2- | 336 | T | Hgn | C | — | | —NH₂ |
| 00026717 | | | | | | | |
| PDC_EphA2- | 337 | T | Hgn | C | | | —NH₂ |
| 00026632 | | | | | | | |
| PDC_EphA2- | 338 | T | Hgn | C | | | —NH₂ |
| 00026715 | | | | | | | |
| PDC_EphA2- | 339 | T | Hgn | C | | | —NH₂ |
| 00026718 | | | | | | | |
| PDC_EphA2- | 340 | T | K(DOTA- | C | | | —NH₂ |
| 00026633 | | | Lu) | | | | |
| PDC_EphA2- | 341 | T | K(DOTA- | C | | | —NH₂ |
| 00026716 | | | Lu) | | | | |
| PDC_EphA2- | 342 | T | K(DOTA- | C | | | —NH₂ |
| 00026719 | | | Lu) | | | | |
| PDC_EphA2- | 343 | T | Hgn | C | dk(DOTA(Lu)) | | —NH₂ |
| 00010011- | | | | | | | |
| C302 | | | | | | | |
| PDC_EphA2- | 344 | T | Hgn | C | bA | dk(PEG2- | —NH₂ |
| 00010011- | | | | | | PEG8- | |
| C304 | | | | | | DOTA(Lu)) | |
| PDC_EphA2- | 345 | T | Hgn | C | dk(DOTA(Lu)) | | —NH₂ |
| 00010012- | | | | | | | |
| C302 | | | | | | | |
| PDC_EphA2- | 346 | T | Hgn | C | bA | dk(PEG2- | —NH₂ |
| 00010012- | | | | | | PEG8- | |
| C304 | | | | | | DOTA(Lu)) | |
| PDC_EphA2- | 347 | T | Hgn | C | dk(DOTA(Lu)) | | —NH₂ |
| 00022595- | | | | | | | |
| C302 | | | | | | | |
| PDC_EphA2- | 348 | T | Hgn | C | dk(DOTA(Lu)) | | —NH₂ |
| 00022612- | | | | | | | |
| C302 | | | | | | | |
| PDC_EphA2- | 349 | T | Hgn | C | dk(DOTA(Lu)) | | —NH₂ |
| 00022614- | | | | | | | |
| C302 | | | | | | | |
| PDC_EphA2- | 350 | T | Hgn | C | dk(DOTA(Lu)) | | —NH₂ |
| 00022615- | | | | | | | |
| C302 | | | | | | | |
| PDC_EphA2- | 351 | T | N | C | dk(DOTA(Lu)) | | —NH₂ |
| 00026424- | | | | | | | |
| C302 | | | | | | | |
| PDC_EphA2- | 352 | T | N | C | dk(DOTA(Lu)) | | —NH₂ |
| 00026425- | | | | | | | |
| C302 | | | | | | | |
| PDC_EphA2- | 353 | T | N | C | dk(DOTA(Lu)) | | —NH₂ |
| 00026426- | | | | | | | |
| C302 | | | | | | | |
| PDC_EphA2- | 354 | T | Hgn | C | bA | dk(DOTA(Lu) | —NH₂ |
| 00010011- | | | | | | | |
| C303 | | | | | | | |
| PDC_EphA2- | 355 | T | Hgn | C | bA | dk(DOTA(Lu) | —NH₂ |
| 00010012- | | | | | | | |
| C303 | | | | | | | |
| PDC_EphA2- | 356 | T | Hgn | C | de | | —OH |
| 00027104 | | | | | | | |
| PDC_EphA2- | 357 | T | Hgn | C | de | | —NH₂ |
| 00027106 | | | | | | | |
| PDC_EphA2- | 358 | T | Hgn | C | de | | —OH |
| 00027103 | | | | | | | |
| PDC_EphA2- | 359 | T | Hgn | C | de | | —NH₂ |
| 00027105 | | | | | | | |
| PDC_EphA2- | 360 | T | Hgn | C | | | —NH₂ |
| 00026528- | | | | | | | |
| 301 | | | | | | | |
| PDC_EphA2- | 361 | T | K(DOTA(Lu)) | C | | | —NH₂ |
| 00026529- | | | | | | | |
| 301 | | | | | | | |

TABLE 2A-Lu177
Exemplary conjugates of the present disclosure
containing a chelated Lutetium-177 (12mer cyclic peptides).
As an example, PDC_EphA2-00001418-C406 of Table 2A-Lu177
has the same structure as PDC_EphA2-00001418-C306 of Table
2A-Lu, except that Lu177 is present in PDC_EphA2-00001418-C406
and Lu175 is present in PDC_EphA2-00001418-C306.

| | Conjugate structure | |
|---|---|---|
| | (See Table 2A-Lu; wherein the Lu-175 | |
| | from a conjugate of Table 2A-Lu is | |
| | replaced with Lu-177) | |
| | Corresponding | Radionuclide |
| | conjugate | bound to the |
| | structure | chelator |
| | from | (replacing |
| Conjugate ID | Table 2A-Lu | Lu-175) |
| PDC_EphA2- | PDC_EphA2-00001418- | ¹⁷⁷Lu |
| 00001418-C406 | C306 | |
| PDC_EphA2- | PDC_EphA2-00001419- | ¹⁷⁷Lu |
| 00001419-C40x | C30x | |
| PDC_EphA2- | PDC_EphA2-00001417- | ¹⁷⁷Lu |
| 00001417-C406 | C306 | |
| PDC_EphA2- | PDC_EphA2-00001417- | ¹⁷⁷Lu |
| 00001417-C404 | C304 | |
| PDC_EphA2- | PDC_EphA2-00001417- | ¹⁷⁷Lu |
| 00001417-C402 | C302 | |
| PDC_EphA2- | PDC_EphA2-00001420- | ¹⁷⁷Lu |
| 00001420-C40x | C30x | |
| PDC_EphA2- | PDC_EphA2-00008093- | ¹⁷⁷Lu |
| 00008093-C410 | C310 | |
| PDC_EphA2- | PDC_EphA2-00008093- | ¹⁷⁷Lu |
| 00008093-C404 | C304 | |
| PDC_EphA2- | PDC_EphA2-00008093- | ¹⁷⁷Lu |
| 00008093-C402 | C302 | |
| PDC_EphA2- | PDC_EphA2-00008094- | ¹⁷⁷Lu |
| 00008094-C404 | C304 | |
| PDC_EphA2- | PDC_EphA2-00007196- | ¹⁷⁷Lu |
| 00007196-C404 | C304 | |
| PDC_EphA2- | PDC_EphA2-00007196- | ¹⁷⁷Lu |
| 00007196-C410 | C310 | |
| PDC_EphA2- | PDC_EphA2-00007196- | ¹⁷⁷Lu |
| 00007196-C402 | C302 | |
| PDC_EphA2- | PDC_EphA2-00007196- | ¹⁷⁷Lu |
| 00007196-C412 | C312 | |
| PDC_EphA2- | PDC_EphA2-00007196- | ¹⁷⁷Lu |
| 00007196-C442 | C342 | |
| PDC_EphA2- | PDC_EphA2-00007196- | ¹⁷⁷Lu |
| 00007196-C432 | C332 | |
| PDC_EphA2- | PDC_EphA2-00007196- | ¹⁷⁷Lu |
| 00007196-C422 | C322 | |
| PDC_EphA2- | PDC_EphA2-00007196- | ¹⁷⁷Lu |
| 00007196-C405 | C305 | |
| PDC_EphA2- | PDC_EphA2-00026772 | ¹⁷⁷Lu |
| 00026772-C400 | | |
| PDC_EphA2- | PDC_EphA2-00007197- | ¹⁷⁷Lu |
| 00007197-C40x | C30x | |
| PDC_EphA2- | PDC_EphA2-00008010- | ¹⁷⁷Lu |
| 00008010-C402 | C302 | |
| PDC_EphA2- | PDC_EphA2-00007198- | ¹⁷⁷Lu |
| 00007198-C402 | C302 | |
| PDC_EphA2- | PDC_EphA2-00007199- | ¹⁷⁷Lu |
| 00007199-C402 | C302 | |
| PDC_EphA2- | PDC_EphA2-00007180- | ¹⁷⁷Lu |
| 00007180-C402 | C302 | |
| PDC_EphA2- | PDC_EphA2-00013693- | ¹⁷⁷Lu |
| 00013693-C404 | C304 | |
| PDC_EphA2- | PDC_EphA2-00013693- | ¹⁷⁷Lu |
| 00013693-C403 | C303 | |
| PDC_EphA2- | PDC_EphA2-00026773 | ¹⁷⁷Lu |
| 00026773-C400 | | |
| PDC_EphA2- | PDC_EphA2-00013693- | ¹⁷⁷Lu |
| 00013693-C402 | C302 | |
| PDC_EphA2- | PDC_EphA2-00013703- | ¹⁷⁷Lu |
| 00013703-C402 | C302 | |
| PDC_EphA2- | PDC_EphA2-00013704- | ¹⁷⁷Lu |
| 00013704-C404 | C304 | |
| PDC_EphA2- | PDC_EphA2-00013704- | ¹⁷⁷Lu |
| 00013704-C402 | C302 | |
| PDC_EphA2- | PDC_EphA2-00013704- | ¹⁷⁷Lu |
| 00013704-C404 | C304 | |
| PDC_EphA2- | PDC_EphA2-00013705- | ¹⁷⁷Lu |
| 00013705-C402 | C302 | |
| PDC_EphA2- | PDC_EphA2-00007196- | ¹⁷⁷Lu |
| 00007196-C406 | C306 | |
| PDC_EphA2- | PDC_EphA2-00007196- | ¹⁷⁷Lu |
| 00007196-C408 | C308 | |
| PDC_EphA2- | PDC_EphA2-00027131 | ¹⁷⁷Lu |
| 00027131-400 | | |
| PDC_EphA2- | PDC_EphA2-00027128 | ¹⁷⁷Lu |
| 00027128-400 | | |
| PDC_EphA2- | PDC_EphA2-00027127 | ¹⁷⁷Lu |
| 00027127-400 | | |
| PDC_EphA2- | PDC_EphA2-00027104 | ¹⁷⁷Lu |
| 00027104-400 | | |
| PDC_EphA2- | PDC_EphA2-00027103 | ¹⁷⁷Lu |
| 00027103-400 | | |
| PDC_EphA2-00019437- | PDC_EphA2-00019437- | ¹⁷⁷Lu |
| C402 | C302 | |
| PDC_EphA2-00026774- | PDC_EphA2-00026774 | ¹⁷⁷Lu |
| 400 | | |
| PDC_EphA2-00022593- | PDC_EphA2-00022593- | ¹⁷⁷Lu |
| C402 | C302 | |
| PDC_EphA2-00022594- | PDC_EphA2-00022594- | ¹⁷⁷Lu |
| C402 | C302 | |
| PDC_EphA2-00019439- | PDC_EphA2-00019439- | ¹⁷⁷Lu |
| C404 | C304 | |
| PDC_EphA2-00019439- | PDC_EphA2-00019439- | ¹⁷⁷Lu |
| C402 | C302 | |
| PDC_EphA2-00019440- | PDC_EphA2-00019440- | ¹⁷⁷Lu |
| C402 | C302 | |
| PDC_EphA2-00019443- | PDC_EphA2-00019443- | ¹⁷⁷Lu |
| C402 | C302 | |
| PDC_EphA2-00019444- | PDC_EphA2-00019444- | ¹⁷⁷Lu |
| C402 | C302 | |
| PDC_EphA2-00022613- | PDC_EphA2-00022613- | ¹⁷⁷Lu |
| C402 | C302 | |
| PDC_EphA2-00026775- | PDC_EphA2-00026775 | ¹⁷⁷Lu |
| C400 | | |
| PDC_EphA2-00026631- | PDC_EphA2-00026631 | ¹⁷⁷Lu |
| C400 | | |
| PDC_EphA2-00026714- | PDC_EphA2-00026714 | ¹⁷⁷Lu |
| C400 | | |
| PDC_EphA2-00026717- | PDC_EphA2-00026717 | ¹⁷⁷Lu |
| C400 | | |
| PDC_EphA2-00026632- | PDC_EphA2-00026632 | ¹⁷⁷Lu |
| C400 | | |
| PDC_EphA2-00026715- | PDC_EphA2-00026715 | ¹⁷⁷Lu |
| C400 | | |
| PDC_EphA2-00026718- | PDC_EphA2-00026718 | ¹⁷⁷Lu |
| C400 | | |
| PDC_EphA2-00026633- | PDC_EphA2-00026633 | ¹⁷⁷Lu |
| C400 | | |
| PDC_EphA2-00026716- | PDC_EphA2-00026716 | ¹⁷⁷Lu |
| C400 | | |
| PDC_EphA2-00026719- | PDC_EphA2-00026719 | ¹⁷⁷Lu |
| C400 | | |
| PDC_EphA2-00010011- | PDC_EphA2-00010011- | ¹⁷⁷Lu |
| C402 | C302 | |
| PDC_EphA2-00010011- | PDC_EphA2-00010011- | ¹⁷⁷Lu |
| C404 | C304 | |
| PDC_EphA2-00010012- | PDC_EphA2-00010012- | ¹⁷⁷Lu |
| C402 | C302 | |
| PDC_EphA2-00010012- | PDC_EphA2-00010012- | ¹⁷⁷Lu |
| C404 | C304 | |
| PDC_EphA2-00022595- | PDC_EphA2-00022595- | ¹⁷⁷Lu |
| C402 | C302 | |
| PDC_EphA2-00022612- | PDC_EphA2-00022612- | ¹⁷⁷Lu |
| C402 | C302 | |
| PDC_EphA2-00022614- | PDC_EphA2-00022614- | ¹⁷⁷Lu |
| C402 | C302 | |
| PDC_EphA2-00022615- | PDC_EphA2-00022615- | ¹⁷⁷Lu |
| C402 | C302 | |
| PDC_EphA2-00026424- | PDC_EphA2-00026424- | ¹⁷⁷Lu |
| C402 | C302 | |
| PDC_EphA2-00026425- | PDC_EphA2-00026425- | ¹⁷⁷Lu |
| C402 | C302 | |
| PDC_EphA2-00026426- | PDC_EphA2-00026426- | ¹⁷⁷Lu |
| C402 | C302 | |
| PDC_EphA2-00010011- | PDC_EphA2-00010011- | ¹⁷⁷Lu |
| C403 | C303 | |
| PDC_EphA2-00010012- | PDC_EphA2-00010012- | ¹⁷⁷Lu |
| C403 | C303 | |
| PDC_EphA2-00007196- | PDC_EphA2-00007196- | ¹⁷⁷Lu |
| C407 | C307 | |
| PDC_EphA2-00007196- | PDC_EphA2-00007196- | ¹⁷⁷Lu |
| C409 | C309 | |
| PDC_EphA2-00027129- | PDC_EphA2-00027129 | ¹⁷⁷Lu |
| 400 | | |
| PDC_EphA2-00027130- | PDC_EphA2-00027130 | ¹⁷⁷Lu |
| 400 | | |
| PDC_EphA2-00027126- | PDC_EphA2-00027126 | ¹⁷⁷Lu |
| 400 | | |
| PDC_EphA2-00027106- | PDC_EphA2-00027106 | ¹⁷⁷Lu |
| 400 | | |
| PDC_EphA2-00027105- | PDC_EphA2-00027105 | ¹⁷⁷Lu |
| 400 | | |

TABLE 2A-Ac225
Exemplary conjugates of the present disclosure
containing a chelated Actinium-225 (12mer cyclic peptides).
As an example, PDC_EphA2-00001418-C506of Table 2A-Ac255
has the same structure as PDC_EphA2-00001418-C306 of Table
2A-Lu, except that Ac225 is present in PDC_EphA2-00001418-C506
and Lu175 is present in PDC_EphA2-00001418-C306.

| | Conjugate structure | |
|---|---|---|
| | (See Table 2A-Lu; wherein the Lu-175 | |
| | from a conjugate of Table 2A-Lu is | |
| | replaced with Ac-225) | |
| | Corresponding | Radionuclide |
| | conjugate | bound to the |
| | structure | chelator |
| | from Table | (replacing |
| Conjugate ID | 2A-Lu | Lu-175) |
| PDC_EphA2- | PDC_EphA2-00001418- | ²²⁵Ac |
| 00001418-C506 | C306 | |
| PDC_EphA2- | PDC_EphA2-00001419- | ²²⁵Ac |
| 00001419-C50x | C30x | |
| PDC_EphA2- | PDC_EphA2-00001417- | ²²⁵Ac |
| 00001417-C506 | C306 | |
| PDC_EphA2- | PDC_EphA2-00001417- | ²²⁵Ac |
| 00001417-C504 | C304 | |
| PDC_EphA2- | PDC_EphA2-00001417- | ²²⁵Ac |
| 00001417-C502 | C302 | |
| PDC_EphA2- | PDC_EphA2-00001420- | ²²⁵Ac |
| 00001420-C50x | C30x | |
| PDC_EphA2- | PDC_EphA2-00008093- | ²²⁵Ac |
| 00008093-C510 | C310 | |
| PDC_EphA2- | PDC_EphA2-00008093- | ²²⁵Ac |
| 00008093-C504 | C304 | |
| PDC_EphA2- | PDC_EphA2-00008093- | ²²⁵Ac |
| 00008093-C502 | C302 | |
| PDC_EphA2- | PDC_EphA2-00008094- | ²²⁵Ac |
| 00008094-C504 | C304 | |
| PDC_EphA2- | PDC_EphA2-00007196- | ²²⁵Ac |
| 00007196-C504 | C304 | |
| PDC_EphA2- | PDC_EphA2-00007196- | ²²⁵Ac |
| 00007196-C510 | C310 | |
| PDC_EphA2- | PDC_EphA2-00007196- | ²²⁵Ac |
| 00007196-C502 | C302 | |
| PDC_EphA2- | PDC_EphA2-00007196- | ²²⁵Ac |
| 00007196-C512 | C312 | |
| PDC_EphA2- | PDC_EphA2-00007196- | ²²⁵Ac |
| 00007196-C542 | C342 | |
| PDC_EphA2- | PDC_EphA2-00007196- | ²²⁵Ac |
| 00007196-C532 | C332 | |
| PDC_EphA2- | PDC_EphA2-00007196- | ²²⁵Ac |
| 00007196-C522 | C322 | |
| PDC_EphA2- | PDC_EphA2-00007196- | ²²⁵Ac |
| 00007196-C505 | C305 | |
| PDC_EphA2- | PDC_EphA2-00026772 | ²²⁵Ac |
| 00026772-C500 | | |
| PDC_EphA2- | PDC_EphA2-00007197- | ²²⁵Ac |
| 00007197-C50x | C30x | |
| PDC_EphA2- | PDC_EphA2-00008010- | ²²⁵Ac |
| 00008010-C502 | C302 | |
| PDC_EphA2- | PDC_EphA2-00007198- | ²²⁵Ac |
| 00007198-C502 | C302 | |
| PDC_EphA2- | PDC_EphA2-00007199- | ²²⁵Ac |
| 00007199-C502 | C302 | |
| PDC_EphA2- | PDC_EphA2-00007180- | ²²⁵Ac |
| 00007180-C502 | C302 | |
| PDC_EphA2- | PDC_EphA2-00013693- | ²²⁵Ac |
| 00013693-C504 | C304 | |
| PDC_EphA2- | PDC_EphA2-00013693- | ²²⁵Ac |
| 00013693-C503 | C303 | |
| PDC_EphA2- | PDC_EphA2-00026773 | ²²⁵Ac |
| 00026773-C500 | | |
| PDC_EphA2- | PDC_EphA2-00013693- | ²²⁵Ac |
| 00013693-C502 | C302 | |
| PDC_EphA2- | PDC_EphA2-00013703- | ²²⁵Ac |
| 00013703-C502 | C302 | |
| PDC_EphA2- | PDC_EphA2-00013704- | ²²⁵Ac |
| 00013704-C504 | C304 | |
| PDC_EphA2- | PDC_EphA2-00013704- | ²²⁵Ac |
| 00013704-C502 | C302 | |
| PDC_EphA2- | PDC_EphA2-00013704- | ²²⁵Ac |
| 00013704-C504 | C304 | |
| PDC_EphA2- | PDC_EphA2-00013705- | ²²⁵Ac |
| 00013705-C502 | C302 | |
| PDC_EphA2- | PDC_EphA2-00007196- | ²²⁵Ac |
| 00007196-C506 | C306 | |
| PDC_EphA2- | PDC_EphA2-00007196- | ²²⁵Ac |
| 00007196-C508 | C308 | |
| PDC_EphA2- | PDC_EphA2-00027131 | ²²⁵Ac |
| 00027131-500 | | |
| PDC_EphA2- | PDC_EphA2-00027128 | ²²⁵Ac |
| 00027128-500 | | |
| PDC_EphA2- | PDC_EphA2-00027127 | ²²⁵Ac |
| 00027127-500 | | |
| PDC_EphA2- | PDC_EphA2-00027104 | ²²⁵Ac |
| 00027104-500 | | |
| PDC_EphA2- | PDC_EphA2-00027103 | ²²⁵Ac |
| 00027103-500 | | |
| PDC_EphA2-00019437- | PDC_EphA2-00019437- | ²²⁵Ac |
| C502 | C302 | |
| PDC_EphA2-00026774- | PDC_EphA2-00026774 | ²²⁵Ac |
| 500 | | |
| PDC_EphA2-00022593- | PDC_EphA2-00022593- | ²²⁵Ac |
| C502 | C302 | |
| PDC_EphA2-00022594- | PDC_EphA2-00022594- | ²²⁵Ac |
| C502 | C302 | |
| PDC_EphA2-00019439- | PDC_EphA2-00019439- | ²²⁵Ac |
| C504 | C304 | |
| PDC_EphA2-00019439- | PDC_EphA2-00019439- | ²²⁵Ac |
| C502 | C302 | |
| PDC_EphA2-00019440- | PDC_EphA2-00019440- | ²²⁵Ac |
| C502 | C302 | |
| PDC_EphA2-00019443- | PDC_EphA2-00019443- | ²²⁵Ac |
| C502 | C302 | |
| PDC_EphA2-00019444- | PDC_EphA2-00019444- | ²²⁵Ac |
| C502 | C302 | |
| PDC_EphA2-00022613- | PDC_EphA2-00022613- | ²²⁵Ac |
| C502 | C302 | |
| PDC_EphA2-00026775- | PDC_EphA2-00026775 | ²²⁵Ac |
| C500 | | |
| PDC_EphA2-00026631- | PDC_EphA2-00026631 | ²²⁵Ac |
| C500 | | |
| PDC_EphA2-00026714- | PDC_EphA2-00026714 | ²²⁵Ac |
| C500 | | |
| PDC_EphA2-00026717- | PDC_EphA2-00026717 | ²²⁵Ac |
| C500 | | |
| PDC_EphA2-00026632- | PDC_EphA2-00026632 | ²²⁵Ac |
| C500 | | |
| PDC_EphA2-00026715- | PDC_EphA2-00026715 | ²²⁵Ac |
| C500 | | |
| PDC_EphA2-00026718- | PDC_EphA2-00026718 | ²²⁵Ac |
| C500 | | |
| PDC_EphA2-00026633- | PDC_EphA2-00026633 | ²²⁵Ac |
| C500 | | |
| PDC_EphA2-00026716- | PDC_EphA2-00026716 | ²²⁵Ac |
| C500 | | |
| PDC_EphA2-00026719- | PDC_EphA2-00026719 | ²²⁵Ac |
| C500 | | |
| PDC_EphA2-00010011- | PDC_EphA2-00010011- | ²²⁵Ac |
| C502 | C302 | |
| PDC_EphA2-00010011- | PDC_EphA2-00010011- | ²²⁵Ac |
| C504 | C304 | |
| PDC_EphA2-00010012- | PDC_EphA2-00010012- | ²²⁵Ac |
| C502 | C302 | |
| PDC_EphA2-00010012- | PDC_EphA2-00010012- | ²²⁵Ac |
| C504 | C304 | |
| PDC_EphA2-00022595- | PDC_EphA2-00022595- | ²²⁵Ac |
| C502 | C302 | |
| PDC_EphA2-00022612- | PDC_EphA2-00022612- | ²²⁵Ac |
| C502 | C302 | |
| PDC_EphA2-00022614- | PDC_EphA2-00022614- | ²²⁵Ac |
| C502 | C302 | |
| PDC_EphA2-00022615- | PDC_EphA2-00022615- | ²²⁵Ac |
| C502 | C302 | |
| PDC_EphA2-00026424- | PDC_EphA2-00026424- | ²²⁵Ac |
| C502 | C302 | |
| PDC_EphA2-00026425- | PDC_EphA2-00026425- | ²²⁵Ac |
| C502 | C302 | |
| PDC_EphA2-00026426- | PDC_EphA2-00026426- | ²²⁵Ac |
| C502 | C302 | |
| PDC_EphA2-00010011- | PDC_EphA2-00010011- | ²²⁵Ac |
| C503 | C303 | |
| PDC_EphA2-00010012- | PDC_EphA2-00010012- | ²²⁵Ac |
| C503 | C303 | |
| PDC_EphA2-00007196- | PDC_EphA2-00007196- | ²²⁵Ac |
| C507 | C307 | |
| PDC_EphA2-00007196- | PDC_EphA2-00007196- | ²²⁵Ac |
| C509 | C309 | |
| PDC_EphA2-00027129- | PDC_EphA2-00027129 | ²²⁵Ac |
| 500 | | |
| PDC_EphA2-00027130- | PDC_EphA2-00027130 | ²²⁵Ac |
| 500 | | |
| PDC_EphA2-00027126- | PDC_EphA2-00027126 | ²²⁵Ac |
| 500 | | |
| PDC_EphA2-00027106- | PDC_EphA2-00027106 | ²²⁵Ac |
| 500 | | |
| PDC_EphA2-00027105- | PDC_EphA2-00027105 | ²²⁵Ac |
| 500 | | |

TABLE 2B
Exemplary conjugates of the present disclosure (10-mer peptides)
“Term” refers to the functional group at the C-terminus.

| | | Conjugate structure | | | | | | | | | | | |
|---|---|---|---|---|---|---|---|---|---|---|---|---|---|
| | | (including a monocyclic peptide and a metal chelator, e.g., | | | | | | | | | | | |
| | | DOTA; in the monocyclic peptide, the amino acid residue at position 1 | | | | | | | | | | | |
| | | and the cysteine residue at position 10 are covalently connected | | | | | | | | | | | |
| | | through —C(═O)—CH₂— bond, | | | | | | | | | | | |
| | | with C═O group attached to the N- | | | | | | | | | | | |
| | | terminal group of amino acid residue at position 1 and CH₂ | | | | | | | | | | | |
| | | attached to the —S— from the cysteine) | | | | | | | | | | | |
| | SEQ ID | From residue position 1 to residue position 10 | | | | | | | | | | | |
| Conjugate ID | NO: | 1 | 2 | 3 | 4 | 5 | 6 | 7 | 8 | 9 | 10 | 11 | Term |
| PDC_EphA2- | 362 | df3CON | Me3Py | N | L | S | MeF | W1Me7Cl | Hcit | W1Me | C | K(DOTA) | —NH₂ |
| 00026600-C201 | | | | | | | | | | | | | |
| PDC_EphA2- | 363 | df3CON | Me3Py | N | L | S | Me3Py | W1Me7Cl | Hcit | W1Me | C | K(DOTA) | —NH₂ |
| 00026601-C201 | | | | | | | | | | | | | |
| PDC_EphA2- | 364 | df3CON | Me3Py | N | Cbg | S | Me3Py | W1Me7Cl | Hcit | W1Me | C | K(DOTA) | —NH₂ |
| 00026602-C201 | | | | | | | | | | | | | |
| PDC_EphA2- | 365 | df3CON | Me3Py | N | Cbg | Qglucamine | Me3Py | W1Me7Cl | KCOpipzaa | W1Me | C | K(DOTA) | —NH₂ |
| 00026603-C201 | | | | | | | | | | | | | |
| PDC_EphA2- | 366 | df3CON | Me3Py | N | Cbg | Qglucamine | Me3Py | W1Me7Cl | Qglucamine | W1Me | C | K(DOTA) | —NH₂ |
| 00026604-C201 | | | | | | | | | | | | | |
| PDC_EphA2- | 367 | df3CON | Me3Py | N | Cbg | Hgn | MeE | W1Me7Cl | KCOpipzaa | W1Me | C | K(DOTA) | —NH₂ |
| 00026606-C201 | | | | | | | | | | | | | |
| PDC_EphA2- | 368 | df3CON | Me3Py | N | Cbg | Qglucamine | MeE | W1Me7Cl | Hcit | W1Me | C | K(DOTA) | —NH₂ |
| 00026607-C201 | | | | | | | | | | | | | |
| PDC_EphA2- | 369 | df3CON | MeF3H | N | Cbg | Hgn | MeE | W1Me7Cl | KCOpipzaa | W1Me | C | K(DOTA) | —NH₂ |
| 00026608-C201 | | | | | | | | | | | | | |

TABLE 2B-Lu
Exemplary conjugates of the present disclosure
(10-mer peptides) including chelated Lu

| | | Radionuclide/metal |
|---|---|---|
| | Conjugate structure | bound to the |
| Conjugate ID | (See Table 2B) | chelator |
| PDC_EphA2- | PDC_EphA2-00026600-C201 | ¹⁷⁵Lu |
| 00026600-C301 | | |
| PDC_EphA2- | PDC_EphA2-00026601-C201 | ¹⁷⁵Lu |
| 00026601-C301 | | |
| PDC_EphA2- | PDC_EphA2-00026602-C201 | ¹⁷⁵Lu |
| 00026602-C301 | | |
| PDC_EphA2- | PDC_EphA2-00026603-C201 | ¹⁷⁵Lu |
| 00026603-C301 | | |
| PDC_EphA2- | PDC_EphA2-00026604-C201 | ¹⁷⁵Lu |
| 00026604-C301 | | |
| PDC_EphA2- | PDC_EphA2-00026606-C201 | ¹⁷⁵Lu |
| 00026606-C301 | | |
| PDC_EphA2- | PDC_EphA2-00026607-C201 | ¹⁷⁵Lu |
| 00026607-C301 | | |
| PDC_EphA2- | PDC_EphA2-00026608-C201 | ¹⁷⁵Lu |
| 00026608-C301 | | |

TABLE 2B-Lu177
Exemplary conjugates of the present disclosure (10-
mer peptides) including chelated lutetium-177

| | | Radionuclide |
|---|---|---|
| | Conjugate structure | bound to the |
| Conjugate ID | (See Table 2B) | chelator |
| PDC_EphA2- | PDC_EphA2-00026600-C201 | ¹⁷⁷Lu |
| 00026600-C401 | | |
| PDC_EphA2- | PDC_EphA2-00026601-C201 | ¹⁷⁷Lu |
| 00026601-C401 | | |
| PDC_EphA2- | PDC_EphA2-00026602-C201 | ¹⁷⁷Lu |
| 00026602-C401 | | |
| PDC_EphA2- | PDC_EphA2-00026603-C201 | ¹⁷⁷Lu |
| 00026603-C401 | | |
| PDC_EphA2- | PDC_EphA2-00026604-C201 | ¹⁷⁷Lu |
| 00026604-C401 | | |
| PDC_EphA2- | PDC_EphA2-00026606-C201 | ¹⁷⁷Lu |
| 00026606-C401 | | |
| PDC_EphA2- | PDC_EphA2-00026607-C201 | ¹⁷⁷Lu |
| 00026607-C401 | | |
| PDC_EphA2- | PDC_EphA2-00026608-C201 | ¹⁷⁷Lu |
| 00026608-C401 | | |

TABLE 2B-Ac225
Exemplary conjugates of the present disclosure (10-
mer peptides) including chelated actinium-225

| | | Radionuclide |
|---|---|---|
| | Conjugate structure | bound to the |
| Conjugate ID | (See Table 2B) | chelator |
| PDC_EphA2- | PDC_EphA2-00026600-C201 | ²²⁵Ac |
| 00026600-C501 | | |
| PDC_EphA2- | PDC_EphA2-00026601-C201 | ²²⁵Ac |
| 00026601-C501 | | |
| PDC_EphA2- | PDC_EphA2-00026602-C201 | ²²⁵Ac |
| 00026602-C501 | | |
| PDC_EphA2- | PDC_EphA2-00026603-C201 | ²²⁵Ac |
| 00026603-C501 | | |
| PDC_EphA2- | PDC_EphA2-00026604-C201 | ²²⁵Ac |
| 00026604-C501 | | |
| PDC_EphA2- | PDC_EphA2-00026606-C201 | ²²⁵Ac |
| 00026606-C501 | | |
| PDC_EphA2- | PDC_EphA2-00026607-C201 | ²²⁵Ac |
| 00026607-C501 | | |
| PDC_EphA2- | PDC_EphA2-00026608-C201 | ²²⁵Ac |
| 00026608-C501 | | |

TABLE 2C
Exemplary conjugates of the present disclosure
“Term” refers to the functional group at the C-terminus.

| | | Conjugate structure | | | | | | | |
|---|---|---|---|---|---|---|---|---|---|
| | SEQ | (including a monocyclic peptide and a metal chelator, e.g., DOTA) | | | | | | | |
| | ID | from residue position 1 to residue position 10, 12, etc. | | | | | | | |
| | NO: | 1 | 2 | 3 | 4 | 5 | 6 | 7 | 8 |
| PDC_EphA2- | 370 | da | Me3Py | N | L | Hgn | MeE | W1Me | KCOpipzaa |
| 00007196- | | | | | | | | | |
| C204 | | | | | | | | | |
| PDC_EphA2- | 371 | da | Me3Py | N | L | Hgn | MeF | W1Me | KCOpipzaa |
| 00008093- | | | | | | | | | |
| C204 | | | | | | | | | |
| PDC_EphA2- | 372 | da | Me3Py | N | L | Hgl | MeE | W1Me | KCOpipzaa |
| 00008094- | | | | | | | | | |
| C204 | | | | | | | | | |
| PDC_EphA2- | 373 | da | Me3Py | N | L | Hgn | MeF | W1Me | KCOpipzaa |
| 00008093- | | | | | | | | | |
| C202 | | | | | | | | | |
| PDC_EphA2- | 374 | da | Me3Py | N | L | Hgn | MeF | W1Me | KCOpipzaa |
| 00008093- | | | | | | | | | |
| C210 | | | | | | | | | |
| PDC_EphA2- | 375 | da | Me3Py | N | L | Hgn | MeE | W1Me | KCOpipzaa |
| 00007196- | | | | | | | | | |
| C202 | | | | | | | | | |
| PDC_EphA2- | 376 | da | Me3Py | N | L | Hgn | MeE | W1Me | KCOpipzaa |
| 00007196- | | | | | | | | | |
| C210 | | | | | | | | | |
| PDC_EphA2- | 377 | da | Me3Py | N | Cbg | Hgn | MeN | W1Me | V |
| 00010011- | | | | | | | | | |
| C202 | | | | | | | | | |
| PDC_EphA2- | 378 | da | Me3Py | N | Cbg | Hgn | Me3Py | W1Me | V |
| 00010012- | | | | | | | | | |
| C202 | | | | | | | | | |
| PDC_EphA2- | 379 | da | MeF | N | Cbg | Hgn | MeN | W1Me | V |
| 00013693- | | | | | | | | | |
| C202 | | | | | | | | | |
| PDC_EphA2- | 380 | da | Me3Py | N | Cbg | Hgn | MeN | W1Me | V |
| 00010011- | | | | | | | | | |
| C204 | | | | | | | | | |
| PDC EphA2- | 381 | da | Me3Py | N | Cbg | Hgn | Me3Py | W1Me | V |
| 00010012- | | | | | | | | | |
| C204 | | | | | | | | | |
| PDC_EphA2- | 382 | da | MeF | N | Cbg | Hgn | MeN | W1Me | V |
| 00013693- | | | | | | | | | |
| C204 | | | | | | | | | |
| PDC_EphA2- | 383 | da | Me3Py | N | Cbg | Hgn | MeN | W1Me | V |
| 00010011- | | | | | | | | | |
| C203 | | | | | | | | | |
| PDC_EphA2- | 384 | da | Me3Py | N | Cbg | Hgn | Me3Py | W1Me | V |
| 00010012- | | | | | | | | | |
| C203 | | | | | | | | | |
| PDC_EphA2- | 385 | da | MeF | N | Cbg | Hgn | MeN | W1Me | V |
| 00013693- | | | | | | | | | |
| C203 | | | | | | | | | |
| PDC_EphA2- | 386 | da | MeF | N | Cbg | Hgn | MeN | W1Me | Qglucamine |
| 00013703- | | | | | | | | | |
| C203 | | | | | | | | | |
| PDC_EphA2- | 387 | da | MeF | N | Cbg | Hgn | Me3Py | W1Me | Qglucamine |
| 00013704- | | | | | | | | | |
| C203 | | | | | | | | | |
| PDC_EphA2- | 388 | da | Me3Py | N | Cbg | Hgn | Me3Py | W1Me | Qglucamine |
| 00013705- | | | | | | | | | |
| C203 | | | | | | | | | |
| PDC_EphA2- | 389 | df3CON | Me3Py | N | Cbg | Hgn | Me3Py | W1Me7Cl | Qglucamine |
| 00019437- | | | | | | | | | |
| C203 | | | | | | | | | |
| PDC_EphA2. | 390 | df3CON | Me3Py | N | Cbg | Hgn | MeN | W1Me7Cl | Qglucamine |
| 00022593- | | | | | | | | | |
| C203 | | | | | | | | | |
| PDC_EphA2 | 391 | df3CON | Me3Py | N | Cbg | Qglucamine | MeN | W1Me7Cl | Qglucamine |
| 00022594- | | | | | | | | | |
| C203 | | | | | | | | | |
| PDC_EphA2- | 392 | df3CON | Me3Py | N | Cbg | Hgn | MeN | W1Me | Qglucamine |
| 00022595- | | | | | | | | | |
| C203 | | | | | | | | | |
| PDC_EphA2- | 393 | df3CON | Me3Py | N | Cbg | Hgn | MeE | W1Me7Cl | KCOpipzaa |
| 00019440- | | | | | | | | | |
| C203 | | | | | | | | | |
| PDC_EphA2- | 394 | df3CON | MeF3CN | N | Cbg | Hgn | MeE | W1Me7Cl | KCOpipzaa |
| 00022612- | | | | | | | | | |
| C203 | | | | | | | | | |
| PDC_EphA2- | 395 | df3CON | MeF3H | N | Cbg | Hgn | MeE | W1Me7Cl | KCOpipzaa |
| 00022613- | | | | | | | | | |
| C203 | | | | | | | | | |
| PDC_EphA2- | 396 | df3CON | MeF3CN | N | Cbg | Hgn | Me3Py | W1Me7Cl | Qglucamine |
| 00022614- | | | | | | | | | |
| C203 | | | | | | | | | |
| PDC_EphA2- | 397 | df3CON | MeF3CN | N | Cbg | Hgn | MeN | W1Me7Cl | Qglucamine |
| 00022615- | | | | | | | | | |
| C203 | | | | | | | | | |
| PDC_EphA2- | 398 | df3CON | Me3Py | N | L | N | Me3Py | W1Me7Cl | Qglucamine |
| 00026424- | | | | | | | | | |
| C203 | | | | | | | | | |
| PDC_EphA2- | 399 | df3CON | Me3Py | N | L | N | MeN | W1Me7Cl | Qglucamine |
| 00026425- | | | | | | | | | |
| C203 | | | | | | | | | |
| PDC_EphA2- | 400 | df3CON | Me3Py | N | L | IN | MeN | W1Me7Cl | Cit |
| 00026426- | | | | | | | | | |
| C203 | | | | | | | | | |
| PDC_EphA2- | 401 | da | Me3Py | N | L | K(DOTA) | MeE | W1Me7Cl | KCOpipzaa |
| 00026628 | | | | | | | | | |
| PDC_EphA2- | 402 | da | Me3Py | N | L | Hgn | MeE | W1Me7Cl | K(DOTA) |
| 00026629 | | | | | | | | | |
| PDC_EphA2- | 403 | da | Me3Py | N | L | Hgn | MeE | W1Me7Cl | KCOpipzaa |
| 00026630 | | | | | | | | | |
| PDC_EphA2- | 404 | df3CON | Me3Py | N | L | K(DOTA) | MeN | W1Me7Cl | Qglucamine |
| 00026708 | | | | | | | | | |
| PDC_EphA2- | 405 | df3CON | Me3Py | N | L | Hgn | MeN | W1Me7Cl | K(DOTA) |
| 00026709 | | | | | | | | | |
| PDC_EphA2- | 406 | df3CON | Me3Py | N | L | Hgn | MeN | W1Me7Cl | Qglucamine |
| 00026710 | | | | | | | | | |
| PDC_EphA2- | 407 | df3CON | Me3Py | N | L | K(DOTA) | MeE | W1Me7Cl | KCOpipzaa |
| 00026711 | | | | | | | | | |
| PDC_EphA2- | 408 | df3CON | Me3Py | N | L | Hgn | MeE | W1Me7Cl | K(DOTA) |
| 00026712 | | | | | | | | | |
| PDC EphA2- | 409 | df3CON | Me3Py | N | L | Hgn | MeE | W1Me7Cl | KCOpipzaa |
| 00026713 | | | | | | | | | |
| PDC_EphA2- | 410 | da | Me3Py | N | L | Hgn | MeE | W1Me | KCOpipzaa |
| 00026768 | | | | | | | | | |
| PDC_EphA2- | 411 | da | MeF | N | Cbg | Hgn | MeN | W1Me | V |
| 00026769 | | | | | | | | | |
| PDC_EphA2- | 412 | df3CON | Me3Py | N | Cbg | Hgn | Me3Py | W1Me7Cl | Qglucamine |
| 00026770 | | | | | | | | | |
| PDC EphA2- | 413 | df3CON | MeF3H | N | Cbg | Hgn | MeE | W1Me7Cl | KCOpipzaa |
| 00026771 | | | | | | | | | |
| PDC_EphA2- | 414 | da | Me3Py | N | L | Hgn | MeE | W1Me | KCOpipzaa |
| 00008010- | | | | | | | | | |
| C202 | | | | | | | | | |

Exemplary conjugates of the present disclosure
“Term” refers to the functional group at the C-terminus.

| | | Conjugate structure | | | | | | |
|---|---|---|---|---|---|---|---|---|
| | SEQ | (including a monocyclic peptide and a metal chelator, e.g., DOTA) | | | | | | |
| | ID | from residue position 1 to residue position 10, 12, etc. | | | | | | |
| | NO: | 9 | 10 | 11 | 12 | 13 | 14 | Term |
| PDC_EphA2- | 370 | W1Me | T | Hgn | C | bA | dk(DOTA- | —NH₂ |
| 00007196- | | | | | | | PEG8c- | |
| C204 | | | | | | | PEG2c) | |
| PDC_EphA2- | 371 | W1Me | T | Hgn | C | bA | dk(DOTA- | —NH₂ |
| 00008093- | | | | | | | PEG8c- | |
| C204 | | | | | | | PEG2c) | |
| PDC_EphA2- | 372 | W1Me | T | Hgn | C | bA | dk(DOTA- | —NH₂ |
| 00008094- | | | | | | | PEG8c- | |
| C204 | | | | | | | PEG2c) | |
| PDC_EphA2- | 373 | W1Me | T | Hgn | C | dk(DOTA) | | —NH₂ |
| 00008093- | | | | | | | | |
| C202 | | | | | | | | |
| PDC_EphA2- | 374 | W1Me | T | Hgn | C | dk(DOTA- | | —NH₂ |
| 00008093- | | | | | | bA-MeG- | | |
| C210 | | | | | | MeG- | | |
| | | | | | | MeG- | | |
| | | | | | | MeG- | | |
| | | | | | | MeG- | | |
| | | | | | | MeG- | | |
| | | | | | | MeG- | | |
| | | | | | | MeG- | | |
| | | | | | | MeG- | | |
| | | | | | | MeG(SEQ | | |
| | | | | | | ID NO: 279)) | | |
| PDC_EphA2- | 375 | W1Me | T | Hgn | C | dk(DOTA) | | —NH₂ |
| 00007196- | | | | | | | | |
| C202 | | | | | | | | |
| PDC_EphA2- | 376 | W1Me | T | Hgn | C | dk(DOTA- | | —NH₂ |
| 00007196- | | | | | | bA-MeG- | | |
| C210 | | | | | | MeG- | | |
| | | | | | | MeG- | | |
| | | | | | | MeG- | | |
| | | | | | | MeG- | | |
| | | | | | | MeG- | | |
| | | | | | | MeG- | | |
| | | | | | | MeG- | | |
| | | | | | | MeG- | | |
| | | | | | | MeG(SEQ | | |
| | | | | | | ID NO: 279)) | | |
| PDC_EphA2- | 377 | W1Me | T | Hgn | C | bA | dk(DOTA) | —NH₂ |
| 00010011- | | | | | | | | |
| C202 | | | | | | | | |
| PDC_EphA2- | 378 | W1Me | T | Hgn | C | bA | dk(DOTA) | —NH₂ |
| 00010012- | | | | | | | | |
| C202 | | | | | | | | |
| PDC_EphA2- | 379 | W1Me | T | Hgn | C | bA | dk(DOTA) | —NH₂ |
| 00013693- | | | | | | | | |
| C202 | | | | | | | | |
| PDC_EphA2- | 380 | W1Me | T | Hgn | C | bA | dk(DOTA- | —NH₂ |
| 00010011- | | | | | | | PEG8c- | |
| C204 | | | | | | | PEG2c) | |
| PDC EphA2- | 381 | W1Me | T | Hgn | C | bA | dk(DOTA- | —NH₂ |
| 00010012- | | | | | | | PEG8c- | |
| C204 | | | | | | | PEG2c) | |
| PDC_EphA2- | 382 | W1Me | T | Hgn | C | bA | dk(DOTA- | —NH₂ |
| 00013693- | | | | | | | PEG8c- | |
| C204 | | | | | | | PEG2c) | |
| PDC_EphA2- | 383 | W1Me | T | Hgn | C | dk(DOTA) | | —NH₂ |
| 00010011- | | | | | | | | |
| C203 | | | | | | | | |
| PDC_EphA2- | 384 | W1Me | T | Hgn | C | dk(DOTA) | | —NH₂ |
| 00010012- | | | | | | | | |
| C203 | | | | | | | | |
| PDC_EphA2- | 385 | W1Me | T | Hgn | C | dk(DOTA) | | —NH₂ |
| 00013693- | | | | | | | | |
| C203 | | | | | | | | |
| PDC_EphA2- | 386 | W1Me | T | Hgn | C | dk(DOTA) | | —NH₂ |
| 00013703- | | | | | | | | |
| C203 | | | | | | | | |
| PDC_EphA2- | 387 | W1Me | T | Hgn | C | dk(DOTA) | | —NH₂ |
| 00013704- | | | | | | | | |
| C203 | | | | | | | | |
| PDC_EphA2- | 388 | W1Me | T | Hgn | C | dk(DOTA) | | —NH₂ |
| 00013705- | | | | | | | | |
| C203 | | | | | | | | |
| PDC_EphA2- | 389 | W1Me | T | Hgn | C | dk(DOTA) | | —NH₂ |
| 00019437- | | | | | | | | |
| C203 | | | | | | | | |
| PDC_EphA2. | 390 | W1Me | T | Hgn | C | dk(DOTA) | | —NH₂ |
| 00022593- | | | | | | | | |
| C203 | | | | | | | | |
| PDC_EphA2 | 391 | W1Me | T | Hgn | C | dk(DOTA) | | —NH₂ |
| 00022594- | | | | | | | | |
| C203 | | | | | | | | |
| PDC_EphA2- | 392 | W1Me | T | Hgn | C | dk(DOTA) | | —NH₂ |
| 00022595- | | | | | | | | |
| C203 | | | | | | | | |
| PDC_EphA2- | 393 | W1Me | T | Hgn | C | dk(DOTA) | | —NH₂ |
| 00019440- | | | | | | | | |
| C203 | | | | | | | | |
| PDC_EphA2- | 394 | W1Me | T | Hgn | C | dk(DOTA) | | —NH₂ |
| 00022612- | | | | | | | | |
| C203 | | | | | | | | |
| PDC_EphA2- | 395 | W1Me | T | Hgn | C | dk(DOTA) | | —NH₂ |
| 00022613- | | | | | | | | |
| C203 | | | | | | | | |
| PDC_EphA2- | 396 | W1Me | T | Hgn | C | dk(DOTA) | | —NH₂ |
| 00022614- | | | | | | | | |
| C203 | | | | | | | | |
| PDC_EphA2- | 397 | W1Me | T | Hgn | C | dk(DOTA) | | —NH₂ |
| 00022615- | | | | | | | | |
| C203 | | | | | | | | |
| PDC_EphA2- | 398 | W1Me | T | N | C | dk(DOTA) | | —NH₂ |
| 00026424- | | | | | | | | |
| C203 | | | | | | | | |
| PDC_EphA2- | 399 | W1Me | T | N | C | dk(DOTA) | | —NH₂ |
| 00026425- | | | | | | | | |
| C203 | | | | | | | | |
| PDC_EphA2- | 400 | W1Me | T | N | C | dk(DOTA) | | —NH₂ |
| 00026426- | | | | | | | | |
| C203 | | | | | | | | |
| PDC_EphA2- | 401 | W1Me | T | Hgn | C | | | —NH₂ |
| 00026628 | | | | | | | | |
| PDC_EphA2- | 402 | W1Me | T | Hgn | C | | | —NH₂ |
| 00026629 | | | | | | | | |
| PDC_EphA2- | 403 | W1Me | T | K(DOTA) | C | | | —NH₂ |
| 00026630 | | | | | | | | |
| PDC_EphA2- | 404 | W1Me | T | Hgn | C | | | —NH₂ |
| 00026708 | | | | | | | | |
| PDC_EphA2- | 405 | W1Me | T | Hgn | C | | | —NH₂ |
| 00026709 | | | | | | | | |
| PDC_EphA2- | 406 | W1Me | T | K(DOTA) | C | | | —NH₂ |
| 00026710 | | | | | | | | |
| PDC_EphA2- | 407 | W1Me | T | Hgn | C | | | —NH₂ |
| 00026711 | | | | | | | | |
| PDC_EphA2- | 408 | W1Me | T | Hgn | C | | | —NH₂ |
| 00026712 | | | | | | | | |
| PDC EphA2- | 409 | W1Me | T | K(DOTA) | C | | | —NH₂ |
| 00026713 | | | | | | | | |
| PDC_EphA2- | 410 | W1Me | T | Hgn | C | dk(DOTA) | | —OH |
| 00026768 | | | | | | | | |
| PDC_EphA2- | 411 | W1Me | T | Hgn | C | dk(DOTA) | | —OH |
| 00026769 | | | | | | | | |
| PDC_EphA2- | 412 | W1Me | T | Hgn | C | dk(DOTA) | | —OH |
| 00026770 | | | | | | | | |
| PDC EphA2- | 413 | W1Me | T | Hgn | C | dk(DOTA) | | —OH |
| 00026771 | | | | | | | | |
| PDC_EphA2- | 414 | W1Me | T | hArg | C | dk(DOTA) | | —NH₂ |
| 00008010- | | | | | | | | |
| C202 | | | | | | | | |

TABLE 3
Structures of exemplary unnatural amino acids that can be
incorporated into a peptide described herein

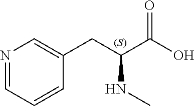

Me3Py

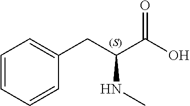

MeF

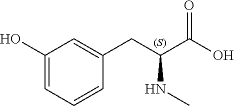

MeF3H

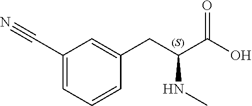

MeF3CN

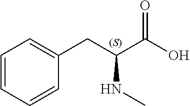

MeF4F

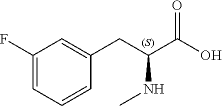

MeF3F

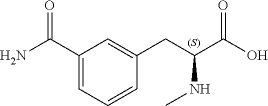

MeF3CON

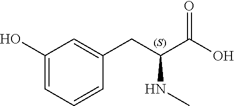

F23dMe

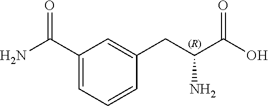

df3CON

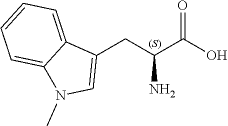

W1Me

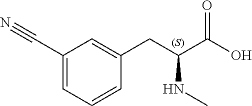

KCOpipzaa

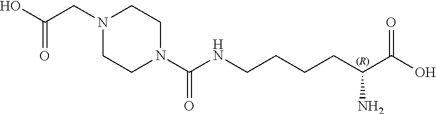

dkCOpipzaa

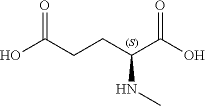

MeE

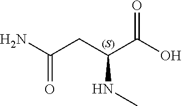

MeN

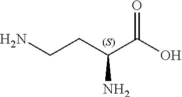

Dab

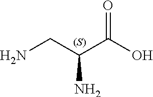

Dap

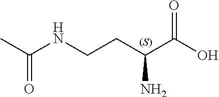

DabAc

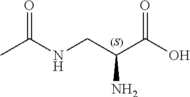

DapAc

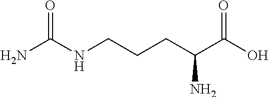 [SMILES: NC(=O)NCCC[C@H](N)C(=O)O]

Cit

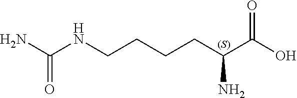

hCit

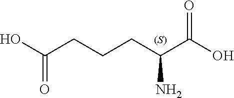

Hgl

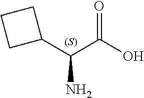

Cbg

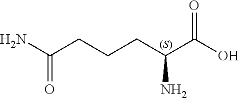

Hgn

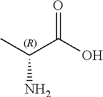

da

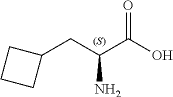

Cba

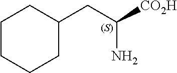

Cha

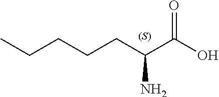

Dahp

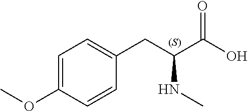

MeY(Me)

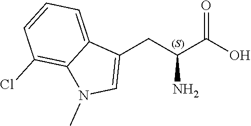

W1Me7Cl

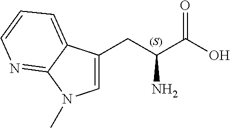

W1Me7N

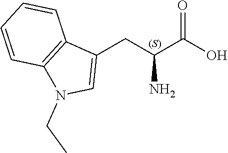

W1Et

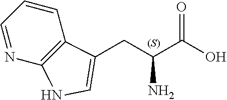

7-AzaTrp

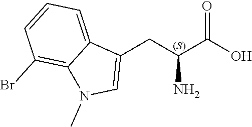

W1Me7Br

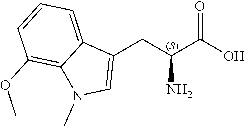

W1Me7OMe

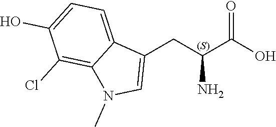

W1Me6O7Cl

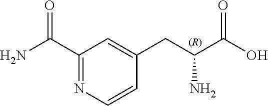

d4PyCON

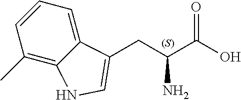

W7Me

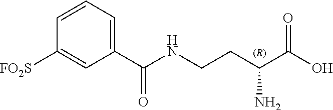

dDap-NH2-Ph3-SO2F

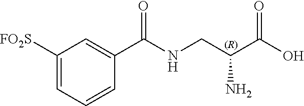

dDap-NH2-Ph3-SO2F

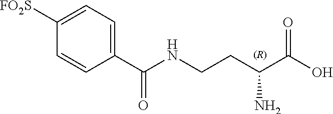

dDap-NH2-Ph4-SO2F

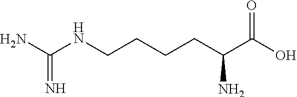 [SMILES: N=C(N)NCCCC[C@H](N)C(=O)O]

hArg

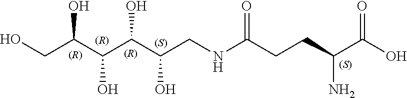

Qglucamine

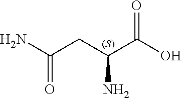

N

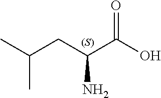

L

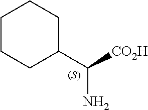

Chg

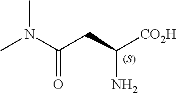

Ndm

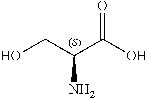

S

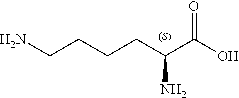

K

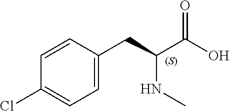

MeF4C

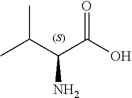

V

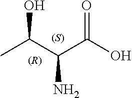

T

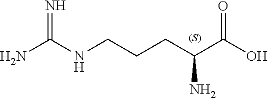

R

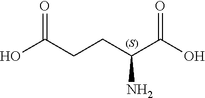

E

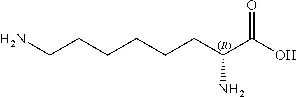

dk

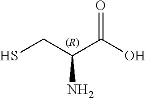

C

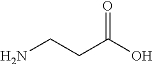

bA

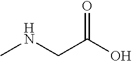

Sar

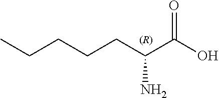

dahp

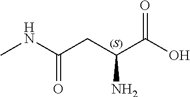

Nmm

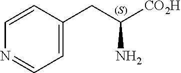

4Py

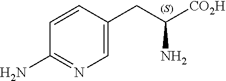

3Py6NH2

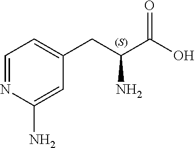

4Py2NH2

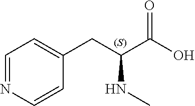

Me4Py

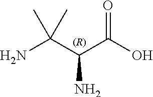

CdMe

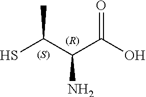

C3SMe

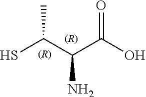

C3RMe

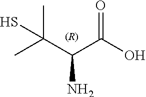

Penicillamine

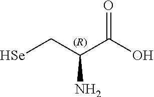

Selenocysteine

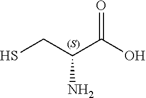

dc

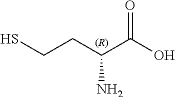

hCys

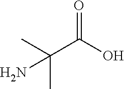

Aib

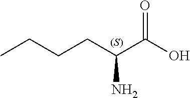

Ahx

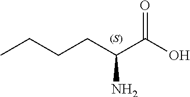

Norleucine

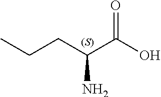

Norvaline

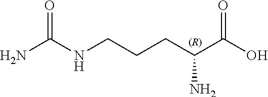

dCit

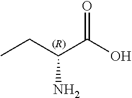

dhAla

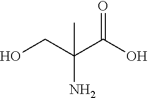

Alpha-methylserine

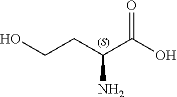

hSer

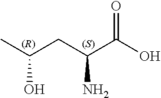

hThr

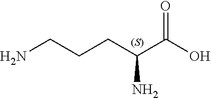

Ornithine

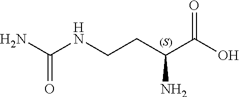

norCit

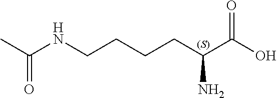

LysAc

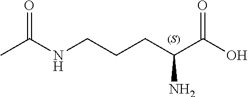

OrnAc

Structures and names of exemplary unnatural amino acids of the present disclosure are further provided below:
Alb(S)-2-amino-3-ureidopropanoic acid (CAS No. 1483-07-4)
da or Da (2R)-2-aminopropanoic acid;
dkCOpipzaa (2R)-2-amino-6-{[4-(carboxymethyl) piperazine-1-carbonyl]amino}hexanoic acid

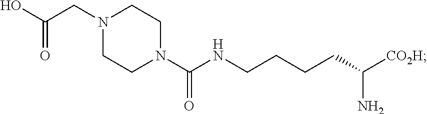 [SMILES: N[C@H](CCCCNC(=O)N1CCN(CC(=O)O)CC1)C(=O)O]

Dahp (2R)-2-aminoheptanoic acid

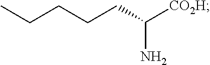

df3CON (2R)-2-amino-3-(3-carbamoylphenyl) propanoic acid (CAS No. 1217637-40-5)

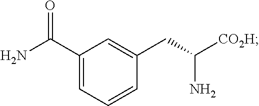 [SMILES: NC(=O)c1cccc(C[C@@H](N)C(=O)O)c1]

MeF (2S)-2-(methylamino)-3-phenylpropanoic acid;
Me3Py (2S)-2-(methylamino)-3-(pyridin-3-yl) propanoic acid (CAS No. 1979173-93-7)

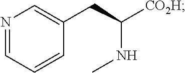

Nal1 1-naphthylalanine;
4Py (2S)-2-amino-3-(pyridin-4-yl) propanoic acid (CAS No. 169555-95-7)

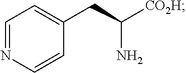 [SMILES: N[C@@H](Cc1ccncc1)C(=O)O]

MeHph (2S)-2-(methylamino)-4-phenylbutanoic acid (CAS No. 1065076-30-3);
W7N (2S)-2-amino-3-{1H-pyrrolo[2,3-b]pyridin-3-yl}propanoic acid (CAS No. 737007-45-3)

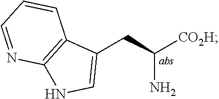 [SMILES: N[C@@H](Cc1c[nH]c2ncccc12)C(=O)O]

QPh (2S)-2-amino-4-(phenylcarbamoyl) butanoic acid (CAS No. 198134-12-2);
MeF3CN (2S)-3-(3-cyanophenyl)-2-(methylamino) propanoic acid (CAS No. 2642331-80-2)

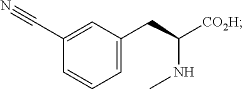

MeF3H (2S)-3-(3-hydroxyphenyl)-2-(methylamino) propanoic acid

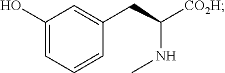

alT (2S,3S)-2-amino-3-hydroxybutanoic acid;
W1Me (2S)-2-amino-3-(1-methyl-1H-indol-3-yl) propanoic acid (CAS No. 1334509-86-2)

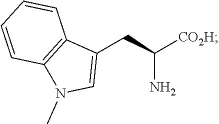 [SMILES: Cn1cc(C[C@H](N)C(=O)O)c2ccccc21]

tma (R)-2-amino-4,4-dimethylpentanoic acid

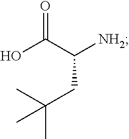

Cbg(S)-2-amino-2-cyclobutylacetic acid (CAS No. 1391630-31-1)

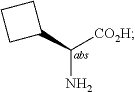

Chg (2S)-2-amino-2-cyclohexylacetic acid (CAS No. 161321-36-4)

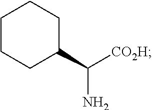 [SMILES: N[C@H](C(=O)O)C1CCCCC1]

Cba (2S)-2-amino-3-cyclobutylpropanoic acid (CAS No. 478183-62-9)

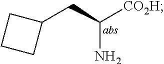

KCOpipzaa (2S)-2-amino-6-{[4-(carboxymethyl) piperazine-1-carbonyl]amino}hexanoic acid

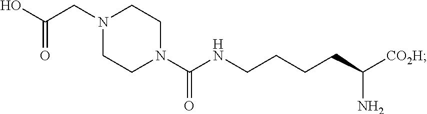 [SMILES: N[C@@H](CCCCNC(=O)N1CCN(CC(=O)O)CC1)C(=O)O]

Hgn (2S)-2-amino-5-carbamoylpentanoic acid (CAS No. 1263046-43-0)

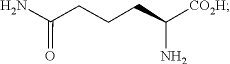 [SMILES: NC(=O)CCC[C@H](N)C(=O)O]

Nmm (2S)-2-amino-3-(methylcarbamoyl) propanoic acid (CAS No. 149204-93-3)

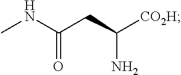

Ndm (2S)-2-amino-3-(dimethylcarbamoyl) propanoic acid (CAS No. 138585-02-1)

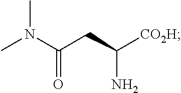

Hcit or hCit (2S)-2-amino-6-(carbamoylamino) hexanoic acid (CAS No. 201485-17-8)

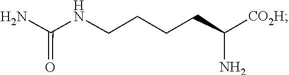 [SMILES: NC(=O)NCCCC[C@H](N)C(=O)O]

Qglucamine (2S)-2-amino-4-{[(2S,3R,4R,5R)-2,3,4,5,6 pentahydroxyhexyl]carbamoyl}butanoic acid

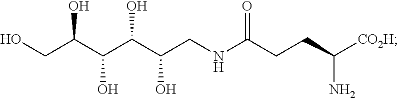 [SMILES: N[C@@H](CCC(=O)NC[C@H](O)[C@@H](O)[C@H](O)[C@H](O)CO)C(=O)O]

mBph 3-phenylphenylalanine;
MeE (2S)-2-(methylamino) pentanedioic acid;
MeN (2S)-3-carbamoyl-2-(methylamino) propanoic acid;
MeF4C (2S)-3-(4-chlorophenyl)-2-(methylamino) propanoic acid (CAS No. 1217779-77-5);
Hph (2S)-2-amino-4-phenylbutanoic acid;
W1Me7N (2S)-2-amino-3-{1-methyl-1H-pyrrolo[2,3-b]pyridin-3-yl}propanoic acid (CAS No. 1813528-10-7)

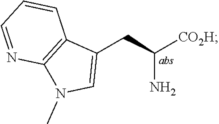 [SMILES: Cn1cc(C[C@H](N)C(=O)O)c2cccnc21]

W1Me7Cl (2S)-2-amino-3-(7-chloro-1-methyl-1H-indol-3-yl) propanoic acid

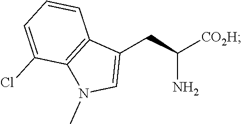 [SMILES: Cn1cc(C[C@H](N)C(=O)O)c2cccc(Cl)c21]

W6C 6-chlorotryptophan
3Py6NH2 (2S)-2-amino-3-(6-aminopyridin-3-yl) propanoic acid

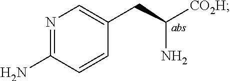 [SMILES: Nc1ccc(C[C@H](N)C(=O)O)cn1]

Cit (2S)-2-amino-5-(carbamoylamino) pentanoic acid

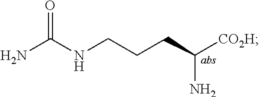 [SMILES: NC(=O)NCCC[C@H](N)C(=O)O]

F23dMe (2S)-2-amino-3-(2,3-dimethylphenyl) propanoic acid (CAS No. 1270295-08-3)

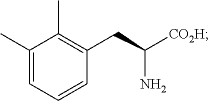

F3C 3-chlorophenylalanine;
Har (2S)-2-amino-6-carbamimidamidohexanoic acid (CAS No. 776277-76-0);
bA 3-aminopropanoic acid;
KAc (2S)-2-amino-6-acetamidohexanoic acid (CAS No. 159766-56-0);
dkAc (2R)-2-amino-6-acetamidohexanoic acid (CAS No. 320410-22-8)
CdMe (R)-2-amino-3-mercapto-3-methylbutanoic acid;
C3SMe (2R,3S)-2-amino-3-mercaptobutanoic acid;
C3RMe (2R,3R)-2-amino-3-mercaptobutanoic acid;
4Py2NH₂ (S)-2-amino-3-(2-aminopyridin-4-yl) propanoic acid; and
Hgl(S)-2-aminohexanedioic acid.

In some embodiments, a peptide described herein has a binding affinity to a human EphA2 of at most 1, 5, 10, 50, 100, 200, 500, 1000, 5000 or 10,000 nM as determined by Kd in surface plasmon resonance (SPR) analysis. In some embodiments, a peptide described herein has a binding affinity to a human EphA2 of at most 100 nM as determined by Kd in surface plasmon resonance (SPR) analysis. In some embodiments, a peptide described herein has a binding affinity to a human EphA2 of at most 1 nM as determined by Kd in surface plasmon resonance (SPR) analysis. In some embodiments, a peptide described herein has a binding affinity to a human EphA2 of at most 2 nM as determined by Kd in surface plasmon resonance (SPR) analysis. In some embodiments, a peptide described herein has a binding affinity to a human EphA2 of at most 5 nM as determined by Kd in surface plasmon resonance (SPR) analysis. In some embodiments, a peptide described herein has a binding affinity to a human EphA2 of at most 10 nM as determined by Kd in surface plasmon resonance (SPR) analysis.

In some embodiments, a conjugate described herein has a binding affinity to a human EphA2 of at most 1, 5, 10, 50, 100, 200, 500, 1000, 5000 or 10,000 nM as determined by Kd in surface plasmon resonance (SPR) analysis. In some embodiments, a conjugate described herein has a binding affinity to a human EphA2 of at most 100 nM as determined by Kd in surface plasmon resonance (SPR) analysis. In some embodiments, a conjugate described herein has a binding affinity to a human EphA2 of at most 1 nM as determined by Kd in surface plasmon resonance (SPR) analysis. In some embodiments, a conjugate described herein has a binding affinity to a human EphA2 of at most 2 nM as determined by Kd in surface plasmon resonance (SPR) analysis. In some embodiments, a conjugate described herein has a binding affinity to a human EphA2 of at most 5 nM as determined by Kd in surface plasmon resonance (SPR) analysis. In some embodiments, a conjugate described herein has a binding affinity to a human EphA2 of at most 10 nM as determined by Kd in surface plasmon resonance (SPR) analysis. In one aspect, the binding affinity of the peptide or radiopharmaceutical conjugate of the present disclosure is at most 100 nM as determined by Kd in surface plasmon resonance (SPR) analysis. In some implementations, the Kd of the peptide or radiopharmaceutical conjugate of the present disclosure is 100 nM ore less, 50 nM or less, 30 nM or less, 20 nM or less, 10 nM or less, 5 nM or less, 4 nM or less, 3 nM or less, 2 nM or less, 1 nM or less, 0.9 nM or less, 0.5 nM or less, 0.4 nM or less, 0.3 nM or less, 0.2 nM or less, 0.1 nM or less, 0.09 nM or less, 0.08 nM or less, 0.07 nM or less, 0.06 nM or less, 0.05 nM or less, 0.04 nM or less, 0.03 nM or less, 0.02 nM or less, 0.01 nM or less.

The molecular weight of the described peptide can vary. In some embodiments, the peptide has a molecular weight of about 0.1 to about 25 kDa. In some embodiments, the peptide has a molecular weight of about 0.2 to about 20 kDa, about 0.5 to about 15 kDa, about 0.75 to about 10 kDa, about 0.5 to about 10 kDa, about 0.5 to about 5 kDa, about 0.5 to about 2.5 kDa, about 0.5 to about 2 kDa, about 0.5 to about 1.5 kDa, about 0.5 to about 1 kDa, about 1 to about 10 kDa, about 1 to about 5 kDa, about 1 to about 2.5 kDa, about 1 to about 2 kDa, about 1 to about 1.5 kDa, about 1 to about 1.25 kDa, or about 0.5 to about 1.25 kDa. In some embodiments, the peptide has a molecular weight of about 0.5 to 5 kDa. In some embodiments, the peptide has a molecular weight of about 0.5 to 2 kDa. In some embodiments, the peptide has a molecular weight of about 0.75 to 1.75 kDa. In some embodiments, the peptide has a molecular weight of about 1 to 1.5 kDa. In some embodiments, the peptide is monocyclic.

A peptide described herein can be cyclized (i.e., macrocyclized). Cyclization can be achieved less ideally via a single disulfide bond, or more ideally via a peptide bond, alkyl bond, alkenyl bond, ester bond, thioester bond, ether bond, thioether bond, phosphate ether bond, azo bond, C—S—C bond, C—N—C bond, C═N—C bond, C═N—O bond, amide bond, lactam bridge, carbamoyl bond, urea bond, thiourea bond, amine bond, thioamide bond, or the like, but not limited to them. In some embodiments, the peptide is a cyclic peptide that is cyclized by a peptide bond, alkyl bond, alkenyl bond, ester bond, thioester bond, ether bond, thioether bond, phosphate ether bond, azo bond, C—N—C bond, C═N—C bond, C═N—O bond, amide bond, lactam bridge, carbamoyl bond, urea bond, thiourea bond, amine bond, or thioamide bond. In some embodiments, the cyclic peptide is cyclized by a thioether bond. In some embodiments, the cyclic peptide is cyclized via an oxime cyclization reaction. A cyclization of a peptide sometimes stabilizes the peptide structure and thereby enhance affinity for a target. The cyclization can occur between the N- and C-terminus, or it can occur between a terminal amino acid and a non-terminal amino acid. In some embodiments, the cyclization occurs between two non-terminal amino acids. In some embodiments, the peptide is cyclized via oxime cyclization. In some embodiments, the peptide is cyclized between cysteine and haloacyl. In some embodiments, the peptide comprises a haloacetyl group (e.g., chloroacetyl or bromoacetyl) at the N-terminus. In some embodiments, the peptide comprises a haloacetyl group (e.g., chloroacetyl or bromoacetyl) at the C-terminus. In some embodiments, the peptide comprises a Cys at the C-terminus. In some embodiments, the peptide comprises a Cys at the N-terminus. In some embodiments, the cyclization occurs via a thioether bond between Cys and a haloacetyl group. In some embodiments, the cyclization occurs between the N-terminus and the C-terminus of the peptide.

As amino acids for macrocyclization, for example, an amino acid having the following functional group A and an amino acid having a corresponding functional group B can be used (see Table 4A). Either the functional group A or the functional group B may be placed on the N-terminal side. The amino acid having the functional group A and the amino acid having the functional group B can each be an N-terminal amino acid or C-terminal amino acid or a non-terminal amino acid. In some embodiments, an amino acid having the functional group A is placed at the N-terminus. In some embodiments, an amino acid having the functional group A is placed at the C-terminus. In some embodiments, an amino acid having the functional group A is placed at a non-terminal amino acid. In some embodiments, an amino acid having the functional group B is placed at the N-terminus. In some embodiments, an amino acid having the functional group B is placed at the C-terminus. In some embodiments, an amino acid having the functional group B is placed at a non-terminal amino acid.

TABLE 4A
Functional groups for cyclization

| | Functional group A | Functional group B |
|---|---|---|
| (I) | 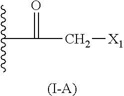 | 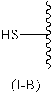 [SMILES: *S ; "(I-B)"] |
| | X₁ is a halogen such as Cl, Br or I | |
| (II) | 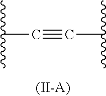 [SMILES: *C#C*] | 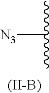 [SMILES: *N=[N+]=[N-] ; "(II-B)"] |
| (III) | 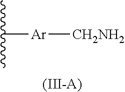 | 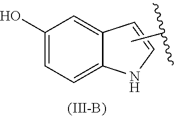 |
| | Ar is substituted or unsubstituted | |
| | aryl or heteroaryl | |
| (IV) | 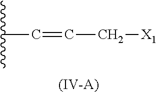 | 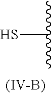 [SMILES: *S ; "(IV-B)"] |
| | X₁ is a halogen such as Cl, Br or I | |
| (V) | 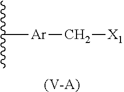 | 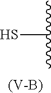 [SMILES: *S ; "(V-B)"] |
| | X₁ is a halogen such as Cl, Br or I; | |
| | Ar is substituted or unsubstituted | |
| | aryl or heteroaryl | |
| (VI) | 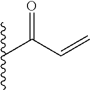 [SMILES: *C(=O)C=C] (VI-A) | 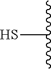 [SMILES: *S] (VI-B) |
| (VII) | 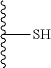 [SMILES: *S] | 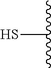 [SMILES: *S] |
| | (VII-A) | (VII-B) |
| (VIII) | 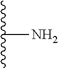 [SMILES: *N] | 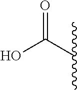 [SMILES: *C(=O)O] |
| | (VIII-A) | (VIII-B) |
| (IX) | 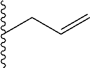 [SMILES: *CC=C] | 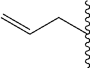 [SMILES: *CC=C] |
| | (IX-A) | (IX-B) |

In some embodiments, as the amino acid (I-A), for example, a chloroacetylated amino acid can be used. Examples of the chloroacetylated amino acids include N-chloroacetyl-L-alanine, N-chloroacetyl-L-phenylalanine, N-chloroacetyl-L-tyrosine, N-chloroacetyl-L-tryptophan, N-3-(2-chloroacetamido)benzoyl-L-phenylalanine, N-3-(2-chloroacetamido)benzoyl-L-tyrosine, N-3-(2-chloroacetamido)benzoyl-L-tryptophan, B—N-chloroacetyl-L-diaminopropanoic acid, γ-N-chloroacetyl-L-diaminobutyric acid, σ-N-chloroacetyl-L-ornithine, ε-N-chloroacetyl-L-lysine, N-3-chloromethylbenzoyl-L-tyrosine, and N-3-chloromethylbenzoyl-L-tryptophane and D-amino acid derivatives corresponding thereto (for example, N-Chloroacetyl-D-alanine, N-Chloroacetyl-D-phenylalanine, N-Chloroacetyl-D-tyrosine, and N-Chloroacetyl-D-tryptophan).

Examples of the amino acid (I-B) include, but are not limited to, cysteine, homocysteine, mercaptonorvaline, mercaptonorleucine, 2-amino-7-mercaptoheptanoic acid, 2-amino-8-mercaptooctanoic acid, and amino acids obtained by protecting the SH group of these amino acids and then eliminating the protecting group, and D-amino acid derivatives corresponding thereto.

The cyclization method can be carried out, for example, according to the method described in Kawakami, T. et al., Nature Chemical Biology 5, 888-890 (2009); Yamagishi, Y. et al., ChemBioChem 10, 1469-1472 (2009); Sako, Y. et al., Journal of American Chemical Society 130, 7932-7934 (2008); or WO2008/117833.

In some embodiments, for example, the amino acid (II-A) is selected from propargylglycine, homopropargylglycine, 2-amino-6-heptynoic acid, 2-amino-7-octynoic acid, and 2-amino-8-nonynoic acid can be used. In addition, 4-pentynoylated or 5-hexynoylated amino acids can also be used. Examples of the 4-pentynoylated amino acids include N-(4-pentenoyl)-L-alanine, N-(4-pentenoyl)-L-phenylalanine, N-(4-pentenoyl)-L-tyrosine, N-(4-pentenoyl)-L-tryptophan, N-3-(4-pentynoylamido)benzoyl-L-phenylalanine, N-3-(4-pentynoylamido)benzoyl-L-tyrosine, N-3-(4-pentynoylamido)benzoyl-L-tryptophan, β-N-(4-pentenoyl)-L-diaminopropanoic acid, γ-N-(4-pentenoyl)-L-diaminobutyric acid, σ-N-(4-pentenoyl)-L-ornithine, and ε-N-(4-pentenoyl)-L-lysine, and D-amino acid derivatives corresponding thereto.

In some embodiments, for example, the amino acid (II-B) is selected from azidoalanine, 2-amino-4-azidobutanoic acid, azidoptonorvaline, azidonorleucine, 2-amino-7-azidoheptanoic acid, and 2-amino-8-azidooctanoic acid can be used. In addition, azidoacetylated or 3-azidopentanoylated amino acids can also be used. Examples of the azidoacetylated amino acids include N-azidoacetyl-L-alanine, N-azidoacetyl-L-phenylalanine, N-azidoacetyl-L-tyrosine, N-azidoacetyl-L-tryptophan, N-3-(4-pentynoylamido)benzoyl-L-phenylalanine, N-3-(4-pentynoylamido)benzoyl-L-tyrosine, N-3-(4-pentynoylamido)benzoyl-L-tryptophan, β-N-azidoacetyl-L-diaminopropanoic acid, γ-N-azidoacetyl-L-diaminobutyric acid, σ-N-azidoacetyl-L-ornithine, and ε-N-azidoacetyl-L-lysine, and D-amino acid derivatives corresponding thereto.

The cyclization method can be performed, for example, according to the method described in Sako, Y. et al., Journal of American Chemical Society 130, 7932-7934 (2008) or WO2008/117833.

Examples of amino acid (III-A) include, but are not limited to, N-(4-aminomethyl-benzoyl)-phenylalanine (AMBF) and 4-3-aminomethyltyrosine.

Examples of the amino acid (III-B) include, but are not limited to, 5-hydroxytryptophan (WoH). The cyclization method can be performed, for example, according to the method described in Yamagishi, Y. et al., ChemBioChem 10, 1469-1472 (2009) or WO2008/117833.

Examples of the amino acid (IV-A) include, but are not limited to, 2-amino-6-chloro-hexynoic acid, 2-amino-7-chloro-heptynoic acid, and 2-amino-8-chloro-octynoic acid.

Examples of the amino acid (IV-B) include, but are not limited to, cysteine, homocysteine, mercaptonorvaline, mercaptonorleucine, 2-amino-7-mercaptoheptanoic acid, and 2-amino-8-mercaptooctanoic acid, amino acids obtained by protecting the SH group of these amino acids and then eliminating the protecting group, and D-amino acid derivatives corresponding thereto. The cyclization method can be performed, for example, according to the method described in WO2012/074129.

Examples of the amino acid (V-A) include, but are not limited to, N-3-chloromethylbenzoyl-L-phenylalanine, N-3-chloromethylbenzoyl-L-tyrosine, and N-3-chloromethylbenzoyl-L-tryptophane.

Examples of the amino acid (V-B) include, but are not limited to, cysteine, homocysteine, mercaptonorvaline, mercaptonorleucine, 2-amino-7-mercaptoheptanoic acid, and 2-amino-8-mercaptooctanoic acid, and amino acids obtained by protecting the SH group of these amino acids and then eliminating the protecting group, and D-amino acid derivatives corresponding thereto.

The amino acids I-A to V-A and I-B to V-B can be introduced into the peptide in a known manner by chemical synthesis or translation and synthesis described herein. In some embodiments, the cyclization reaction comprises forming a thioether bond using an amino acid comprising a sulfanyl group, e.g., cysteine, homocysteine, mercaptonorvaline, mercaptovaline, mercaptonorleucine, 2-amino-7-mercaptoheptanoic acid, and 2-amino-8-mercaptooctanoic acid.

A peptide described herein can comprise one or more negatively charged amino acids and/or one or more positively charged amino acids. Positively charged amino acids include, for example, lysine, arginine, histidine, and amino acids that contain additional amine groups. Positively charged amino acids can comprise a heteroaryl substitution such as pyridine, imidazole, pyrazole, or triazole that has one or more ring nitrogen atoms. Negatively charged amino acids include, for example, amino acids that contain an additional carboxylic acid group such as glutamic acid or the like.

In some embodiments, a cyclic peptide of Formula (I), Formula (I-1), Formula (I-2), Formula (Ia), Formula (Ib), or Formula (Ic) has a net charge of −3 to +1. In some embodiments, the cyclic peptide has a net charge of −3. In some embodiments, the cyclic peptide has a net charge of −2. In some embodiments, the cyclic peptide has a net charge of −1. In some embodiments, the cyclic peptide has a net charge of 0. In some embodiments, the cyclic peptide has a net charge of +1. In some embodiments, a cyclic peptide of Formula (I), Formula (I-1), Formula (I-2), Formula (Ia), Formula (Ib), or Formula (Ic) has a net charge of at most −4. In some embodiments, the cyclic peptide has a net charge of −4. In some embodiments, a cyclic peptide of Formula (I), Formula (I-1), Formula (I-2), Formula (Ia), Formula (Ib), or Formula (Ic) has a net charge of at least +2. In some embodiments, the cyclic peptide has a net charge of +2. In some embodiments, the cyclic peptide has a net charge of +3. The net charge can be determined by aggregating the charge of each of the X1 to X12 amino acids (or each of the amino acid in the peptide). For example, aspartic acid (D) and glutamic acid (E) each has a charge of −1, lysine (K), arginine (R) and histidine (H) each has a charge of +1, and the rest of the canonical amino acids each has a charge of 0.

In some embodiments, a cyclic peptide of formula (I) has a net charge of −3 to +1. In some embodiments, the cyclic peptide has a net charge of −3. In some embodiments, the cyclic peptide has a net charge of −2. In some embodiments, the cyclic peptide has a net charge of −1. In some embodiments, the cyclic peptide has a net charge of 0. In some embodiments, the cyclic peptide has a net charge of +1. The net charge can be determined by aggregating the charge of each of the amino acids of the cyclic peptide.

In some embodiments, a cyclic peptide described herein (e.g., a cyclic peptide of Formula (I), Formula (I-1), Formula (I-2), Formula (Ia), Formula (Ib), or Formula (Ic)) is configured to bind to EphA2 with a prescribed affinity, for example, measured as Plasma Protein Albumin Binding (PPB) percentage. The % bound can be determined by HSA-HPLC method (measurement of drug protein binding by immobilized human serum albumin-HPLC). PPB can be determined in vitro by HPLC (e.g., Example B3) or by other suitable means known in the art. In some embodiments, 1% to 99% of the cyclic peptide binds to Human Serum Albumin (HSA) in vitro as determined by HPLC, according to the conditions described in Example B3. In some embodiments, about 2% to about 99%, about 5% to about 99%, about 10% to about 99%, about 20% to about 99%, about 30% to about 99%, about 40% to about 99%, about 50% to about 99%, about 60% to about 99%, about 70% to about 99%, or about 80% to about 99% of the cyclic peptide binds to HSA in vitro as determined by HPLC. In some embodiments, about 10% to about 95% of the cyclic peptide binds to HSA in vitro (i.e., PPB of about 10% to about 95%). In some embodiments, about 20% to about 90% of the cyclic peptide binds to HSA in vitro. In some embodiments, about 20% to about 60% of the cyclic peptide binds to HSA in vitro. In some embodiments, about 40% to about 95% of the cyclic peptide binds to HSA in vitro. In some embodiments, about 40% to about 80% of the cyclic peptide binds to HSA in vitro. In some embodiments, about 40% to about 60% of the cyclic peptide binds to HSA in vitro. In some embodiments, about 60% to about 99% of the cyclic peptide binds to HSA in vitro. In some embodiments, about 60% to about 95% of the cyclic peptide binds to HSA in vitro. In some embodiments, about 60% to about 80% of the cyclic peptide binds to HSA in vitro. In some embodiments, about 60% to about 70% of the cyclic peptide binds to HSA in vitro. In some embodiments, about 40% to about 50% of the cyclic peptide binds to HSA in vitro. In some embodiments, about 50% to about 60% of the cyclic peptide binds to HSA in vitro. In some embodiments, about 70% to about 80% of the cyclic peptide binds to HSA in vitro. In some embodiments, about 80% to about 99% of the cyclic peptide binds to HSA in vitro. In some embodiments, about 80% to about 85% of the cyclic peptide binds to HSA in vitro.

In some embodiments, a conjugate described herein (e.g., a conjugate comprising a cyclic peptide of Formula (I), Formula (I-1), Formula (I-2), Formula (Ia), Formula (Ib), or Formula (Ic)) is configured to bind to a plasma protein with a prescribed affinity, for example, measured as Plasma Protein Albumin Binding (PPB) percentage. PPB can be determined in vitro by HPLC (e.g., Example B3) or by other suitable means known in the art. In some embodiments, 1% to 99% of the conjugate binds to Human Serum Albumin (HSA) in vitro as determined by HPLC, according to the conditions described in Example B3. In some embodiments, about 2% to about 99%, about 5% to about 99%, about 10% to about 99%, about 20% to about 99%, about 30% to about 99%, about 40% to about 99%, about 50% to about 99%, about 60% to about 99%, about 70% to about 99%, or about 80% to about 99% of the conjugate binds to HSA in vitro as determined by HPLC. In some embodiments, about 10% to about 95% of the conjugate binds to HSA in vitro (i.e., PPB of about 10% to about 95%). In some embodiments, about 20% to about 90% of the conjugate binds to HSA in vitro. In some embodiments, about 20% to about 60% of the conjugate binds to HSA in vitro. In some embodiments, about 40% to about 95% of the conjugate binds to HSA in vitro. In some embodiments, about 40% to about 80% of the conjugate binds to HSA in vitro. In some embodiments, about 40% to about 60% of the conjugate binds to HSA in vitro. In some embodiments, about 60% to about 99% of the conjugate binds to HSA in vitro. In some embodiments, about 60% to about 95% of the conjugate binds to HSA in vitro. In some embodiments, about 60% to about 80% of the conjugate binds to HSA in vitro. In some embodiments, about 60% to about 70% of the conjugate binds to HSA in vitro. In some embodiments, about 40% to about 50% of the conjugate binds to HSA in vitro. In some embodiments, about 50% to about 60% of the conjugate binds to HSA in vitro. In some embodiments, about 70% to about 80% of the conjugate binds to HSA in vitro. In some embodiments, about 80% to about 99% of the conjugate binds to HSA in vitro. In some embodiments, about 80% to about 85% of the conjugate binds to HSA in vitro.

In some embodiments, a cyclic peptide of Formula (I), Formula (I-1), Formula (I-2), Formula (Ia), Formula (Ib), or Formula (Ic) does not contain any S—S bond.

In some embodiments, a peptide of the present disclosure can be cyclized by forming a group as illustrated in Table 4B.

TABLE 4B
Ring Closing Groups
(m and n are independently 0 or an integer from 1 to 6.)

—C(═O)—CH₂—
—C(═O)—CH₂—S—
—C(═O)—CH₂—S—CH₂—
—C(═O)—CH₂—S—CH₂—CH₂—
—(CH₂)ₘ-NH—CO—(CH₂)ₙ-
—(CH₂)ₘ-CO—NH—(CH₂)ₙ-
—(CH₂)ₘ-S—(CH₂)ₙ-
—(CH₂)ₘ-CH═CH—(CH₂)ₙ-
—(CH₂)ₘ-NH—(CH₂)ₙ-
—(CH₂)ₘ-S—CH₂-benzene-CH₂—S—(CH₂)ₙ-
—(CH₂)ₘ-triazole-(CH₂)ₙ-
—(CH₂)ₘ-succinimide-S—(CH₂)ₙ-
—C(═O)—CH₂—NH—CH₂—
—C(═O)—CH₂—O—CH₂—
—C(═O)—CH₂—CH₂—S—
—(CH₂)ₘ-S—S—(CH₂)ₙ-
—(CH₂)ₘ-C(═O)—NH—(CH₂)ₙ-
—(CH₂)ₘ-CH₂—CH₂—(CH₂)ₙ-

In some embodiments, m is 0 and n is 0. In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, a peptide of the present disclosure, e.g., peptides of Formulas (I), (Ia), (Ib) and (Ic), can be cyclized by reacting a first functional group with a second functional group, see Table 4C. In some embodiments, the first functional group is located at the N-terminus. In some embodiments, the first functional group is located at a non-terminal amino acid. In some embodiments, the second functional group is located at the C-terminus. In some embodiments, the second functional group is located at a non-terminal amino acid.

TABLE 4C
Formation of Ring Closing Groups

| | Second Functional group (or amino acid) |
|---|---|
| First Functional group | (e.g., at C-terminus or at a |
| (e.g., at N-terminus) | non-terminal amino acid) |
| —C(═O)—CH₂Cl | Cysteine, homocysteine, lysine, homolysine, |
| | ornithine, diaminobutric acid, serine, |
| | homoserine, threonine, homothreonine |
| —(CH₂)ₙ-NH₂ | Aspartic acid, glutamic acid, |
| | homoglutamic acid |
| —(CH₂)ₙ-CO₂H | Lysine, homolysine, ornithine, |
| | diaminobutric acid, |
| —(CH₂)ₙ-Br | Cysteine, homocysteine |
| —(CH₂)ₙ-CH═CH₂ | Allyl-glycine, homoallyl-glycine |
| —(CH₂)ₙ-NH₂ | Aspartate-4-semialdehyde, |
| | glutamate-5-semialdehyde |
| —(CH₂)ₙ-SH | Cysteine, homocysteine conjugated with |
| | 1,2- or 1,3- or 1,4-bis-(bromomethyl)benzene |
| —(CH₂)ₙ-alkyne | XaaC is an amino acid with a side |
| | chain with azide |
| —(CH₂)ₙ-N₃ | Alkynyl-glycine, homoalkynyl-glycine |
| —(CH₂)ₙ-maleimide | Cysteine, homocysteine |

In some embodiments, a conjugate comprising any one of peptide of Table 1 may further comprise amino acid residues at the N and/or C terminus of the peptide, which is not part of the cyclic structure. In some embodiments, the conjugate further comprises a metal chelator and optionally a linker. In some embodiments, the conjugate further comprises a radionuclide such as Ac-225 or Lutetium-177. In some embodiments, the conjugate further comprises a covalent radionuclide, and optionally a linker connecting the peptide and the covalent radionuclide. In some embodiments, the conjugate further comprises a covalent radionuclide such as ¹⁸F, ⁷⁴As, ⁷⁶Br, ¹²³I, ¹²⁴I, ¹²⁵I, ¹³¹I, or ²¹¹At.

A peptide described herein can be a peptide mimetic. For example, the peptide can comprise non-peptide bonds and it can comprise one or more unnatural amino acids. Unless stated otherwise, each of the amino acid in a peptide described herein (except the natural amino acid glycine) can independently be in its D or L form. Both D and L forms are encompassed by the present disclosure.

In the present disclosure, the term amino acid embraces derivatives of amino acids. The derivatives include, for example, amino acids obtained by modifying a natural amino acid constituting a protein produced by cellular DNA-encoded biological matter. Examples of such non-natural amino acids include hydroxyproline and hydroxylysine, which are amino acids having a hydroxyl group introduced therein, and diaminopropionic acid, which is an amino acid having an amino group introduced therein.

A peptide described herein can comprise an N-substituted amino acid. In some embodiments, the N-substituted amino acid is a derivative of tryptophan, phenylalanine, tyrosine, arginine, histidine, isoleucine, leucine, lysine, or valine. In some embodiments, the N-substitution is an N-alkyl, such as N-methyl and N-ethyl. In some embodiments, the N-substitution is N-methyl. In some embodiments, the N-substitution is an N-aryl, such as N-phenyl or N-biphenyl. In some embodiments, the N-substitution is an N-heteroaryl such as N-pyridyl. In some embodiments, the N-substituted amino acid is at the N-terminus of the peptide. In some embodiments, the N-substituted amino acid is a non-terminal amino acid.

In some embodiments, peptides described herein comprise one or more amino acids in Tables 5A to 5F.

TABLE 5A
Exemplary Amino Acids at N or C-terminus

| | |
|---|---|
| N-Chloroacetyl-L-alanine | Acetyl-L-alanine |
| N-Chloroacetyl-L-phenylalanine | Acetyl-L-phenylalanine |
| N-Chloroacetyl-L-phenylalanine | Acetyl-L-tyrosine |
| N-Chloroacetyl-L-tyrosine | Acetyl-L-tryptophan |
| N-Chloroacetyl-L-tryptophan | Acetyl-D-alanine |
| N-Chloroacetyl-D-alanine | Acetyl-D-phenylalanine |
| N-Chloroacetyl-D-phenylalanine | Acetyl-D-tyrosine |
| N-Chloroacetyl-D-tyrosine | Acetyl-D-tryptophan |
| N-Chloroacetyl-D-tryptophan | N-3-chloromethylbenzoyl-L-tyrosine |
| | N-3-chloromethylbenzoyl-L-tryptophan |

TABLE 5B

Exemplary Amino Acids That
Crosslink With A Peptide
Nγ-(2-chloroacetyl)-α,γ-diaminobutylic acid
Nγ-(2-chloroacetyl)-α,γ-diaminopropanoic acid

TABLE 5C
D-amino Acids

D-Serine
D-Phenylalanine
D-Tyrosine
D-Tryptophan

TABLE 5D
Exemplary N-alkylamino Acids

N-alkyl-Glycine
N-alkyl-Alanine
N-alkyl-Phenylalanine
N-alkyll-Tyrosine
N-alkyl-Serine
N-alkyll-Histidine
N-alkyl-Tryptophan

Exemplary alkyl groups for Table 5D include methyl, ethyl, and propyl groups.

TABLE 5E
Exemplary Peptoid Blocks

N-ethyl-Glycine
N-n-propyl-Glycine
N-n-butyl-Glycine
N-n-pentyl-Glycine
N-n-hexyl-Glycine
N-n-heptyl-Glycine
N-n-octyl-Glycine
N-isopentyl-Glycine
N-(2-phenylethyl)-Glycine
N-(3-phenylpropyl)-Glycine
N-[2-(p-hydroxyphenyl)ethyl]-Glycine

TABLE 5F
Exemplary Unnatural Amino Acids

p-biphenylalanine
p-trifluoromethylphenylalanine
p-azidophenylalanine
p-biotinyl-aminophenylalanine
e-N-Biotinyl-lysine
e-N-Acetyl-lysine
L-Citrulline
L-5-Hydroxytryptphan
L-1,2,3,4,-Tetrahydroisoquinoline-3-carboxylic
acid
Aminoisobutyric acid
N-methyl-aminoisobutyric acid
N-methyl-Phenylglycine

Amino acids used in the disclosed peptides can be substituted with similar amino acids. In some embodiments, an amino acid can be substituted with another amino acid with similar hydrophobicity. In some embodiments, an amino acid can be substituted with another amino acid with similar hydrophilicity. In some embodiments, an amino acid can be substituted with another amino acid with similar size. In some embodiments, an amino acid can be substituted with another amino acid with similar charge. In some embodiment, an amino acid can be substituted with another amino acid with a similar functional group. In some embodiments, an amino acid can be substituted with another amino acid with the same functional group.

In some embodiments, an amino acid described herein can be replaced with a variant thereof. Examples of an amino acid substitution or variant include derivatives having an amine, amide, ester, or carboxyl group as the C-terminus and/or N-terminus thereof. Additional examples of amino acid/peptide variants include those obtained by modification such as phosphorylation, alkylation (e.g., methylation), acetylation, adenylylation, ADP-ribosylation, or glycosylation and fused protein obtained by fusion with another peptide or protein. These variants can be prepared by those skilled in the art in a known manner or a method based thereon. An amino acid variant further encompasses the amino acids that have the same functional groups but with different lengths of the side chain (e.g., LysAc vs. OrnAc and cysteine vs. homocysteine). An amino acid variant further encompasses amino acids with a different aromatic moiety compared to the canonical amino acid (e.g., the indole in tryptophan vs the 7-azaindole in 7-AzaTrp; the phenyl in phenylalanine vs the pyridine in 4Py). An amino acid variant further encompasses amino acids with optional substituents, i.e., optionally substituted amino acid. In some embodiments, the optionally substituted amino acid is optionally substituted with one or more substituents independently selected from halogen, hydroxyl, cyano, amino, amide, nitro, ureido, C₁-C₆ alkyl, C₁-C₆ alkoxy, C₆-C₁₀ aryl, C₃-C₆ cycloalkyl, 6-10 membered heterocycloalkyl, and 6-10 membered heteroaryl. In some embodiments, the optionally substituted amino acid is optionally substituted with one or more substituents independently selected from halogen, —CN, —NH₂, —NH(alkyl), —N(alkyl)₂, oxo, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), SF₅, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O) R^{a}, —R^{b}—C(O)OR^{a}, —R^{b}—C(O)N(R^{a})₂, —R^{b}—O—R^{c}—C(O)N(R^{a})₂, —R^{b}—N (R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O) R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R a, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, and heterocycle, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O) R^{a}, —R^{b}—C(O)O R^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

In some embodiments, a variant of an amino acid is selected from amino acids having one, two or three substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, —S(═O)₂NH₂, —S(═O)₂NH(C₁-C₃alkyl), —S(═O)₂N(C₁-C₃alkyl)₂, C₁-C₆ alkyl, C₁-C₆ heteroalkyl, C₁-C₆ alkoxy, C₆-C₁₀ aryl, C₃-C₆ cycloalkyl, 6-10 membered heterocycloalkyl, and 6-10 membered heteroaryl.

In some embodiments, the variant is selected from amino acids having one or two substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C1-C₃alkyl), —C(═O)N(C₁-C₃alkyl)₂, and C₁-C₆ alkyl. In some embodiments, the variant is selected from amino acids having one or two substituents based on the amino acid, and wherein the substituents are independently selected from halogen, —CN, —NH₂, —NH(C₁-C₃alkyl), —N(C₁-C₃alkyl)₂, and C₁-C₆ alkyl. In some embodiments, the variant is selected from amino acids having one or two substituents based on the amino acid, and wherein the substituents are independently selected from C₁-C₆ alkyl.

In some embodiments, a variant of an amino acid is selected from amino acids that have the similar hydrophilicity or hydrophobicity compared to the amino acid. Thus, in some embodiments, a positively charged amino acid can be a variant of another positively charged amino acid. In some embodiments, a negatively charged amino acid can be a variant of another negatively charged amino acid. In some embodiments, a zwitterionic amino acid can be a variant of another zwitterionic amino acid.

In some embodiments, a hydrophilic amino acid has an electrically charged side chain. In some embodiments, a hydrophilic amino acid has a positive charge. In some embodiments, a hydrophilic amino acid has a negative charge. In some embodiments, a hydrophilic amino acid is zwitterionic (e.g., KCOpipzaa). In some embodiments, a hydrophilic amino acid comprises a —OH, COOH, —NH— or NH₂ moiety. In some embodiments, a hydrophilic amino acid comprises —OH, —C(O)OH, —NHC(═NH)NH₂, —NHC(O)NH₂, —C(O)NH₂, or —NHC(O)CH₃. In some embodiments, a hydrophilic amino acid comprises a side chain of C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl.

In some embodiments, a hydrophobic amino acid is not charged. In some embodiments, a hydrophobic amino acid contains at least 2 contiguous carbon atoms. In some embodiments, a hydrophobic amino acid comprises at least 3 contiguous carbon atoms, either linear or branched. In some embodiments, a hydrophobic amino acid comprises at least 4 contiguous carbon atoms, either linear or branched. In some embodiments, a hydrophobic amino acid comprises at least 5 contiguous carbon atoms, either linear or branched. In some embodiments, a hydrophobic amino acid comprises an ethylene moiety in the side chain. In some embodiments, a hydrophobic amino acid comprises a propylene moiety in the side chain. In some embodiments, a hydrophobic amino acid comprises a butylene moiety in the side chain. In some embodiments, a hydrophobic amino acid comprises phenyl moiety. In some embodiments, a hydrophobic amino acid comprises a heteroaryl moiety. In some embodiments, a hydrophobic amino acid is Trp, Tyr, Phe, or derivatives thereof.

In some embodiments, a variant of an amino acid is selected from amino acids that have the same functional group as the amino acid, and wherein the variant has a different length of a side chain compared to the amino acid. In some embodiments, a variant of an amino acid is selected from amino acids that have the same functional group as the amino acid, and wherein the variant has a different carbon chain length of a side chain compared to the amino acid (e.g., leucine vs. (S)-2-amino-5-methylhexanoic acid, or 2-(methylamino) pentanedioic acid vs. 2-(methylamino) hexanedioic acid). In some embodiments, a variant of an amino acid is selected from amino acids that have the same charge compared to the amino acid. In some embodiments, a variant of an amino acid is selected from amino acids that have the same polarity compared to the amino acid. In some embodiments, an amino acid comprising an aromatic group can be a variant of another amino acid having an aromatic group. In some embodiments, an amino acid comprising a phenyl can be a variant of another amino acid having a phenyl. In some embodiments, an amino acid comprising a heteroaryl can be a variant of another amino acid having a heteroaryl. In some embodiments, an amino acid comprising a heteroaryl can be a variant of another amino acid having a phenyl group. Amino acids having an aromatic group include, but are not limited to, F, W, Me3Py, MeF, MeF3H, MeFCN, MeF4F, MeF3F, MeFCON, F23dMe, df3CON, W1Me, W1Me7Cl, W1Me7N, W1Et, 7-AzaTrp, W1Me7Br, W1Me7Ome, W1Me6O7Cl, d4PyCON, W7Me, dDab-NH₂-Ph3-SO₂F, dDap-NH₂-Ph3-SO₂F, dDap-NH₂-Ph4-SO₂F, MeF4C, 4Py, 3Py6NH2, 4Py2NH₂, and Me4Py. Accordingly, a variant of an amino acid comprising a heteroaryl ring encompasses amino acids comprising a different heteroaryl. In some embodiments, F or a variant thereof encompasses amino acids where the phenyl ring is replaced with a heteroaryl (e.g., pyridine). In some embodiments, an amino acid comprising a cycloalkyl group can be a variant of another amino acid having a cycloalkyl group. In some embodiments, an amino acid comprising a heterocycloalkyl group can be a variant of another amino acid having a heterocycloalkyl group.

In some embodiments, a variant of an amino acid is selected from amino acids that have similar polarity and/or charge with the amino acid. For example, in some embodiments, a polar, uncharged amino acid can be a variant of another polar, uncharged amino acid (e.g., Hgn, Q, S, T, Qglucamine).

In some embodiments, a variant of an amino acid has the same number of hydrogen donor as the amino acid. In some embodiments, a variant of an amino acid has the same number of hydrogen acceptor as the amino acid.

In some embodiments, the variant has a molecular weight that does not vary for more than 14, 28, 30, 45 or 60 g/mol compared to the amino acid. In some embodiments, the variant has a molecular weight that does not vary for more than 14 g/mol compared to the amino acid. In some embodiments, the variant has a molecular weight that does not vary for more than 50 g/mol compared to the amino acid. In some embodiments, the variant has a molecular weight that does not vary for more than 28 g/mol compared to the amino acid.

An amino acid variant further encompasses amino acids wherein a functional group is substituted with another functional group having similar properties, e.g., a cysteine can be substituted with a homocysteine. In some embodiments, an aryl functional group can be substituted with an aryl or heteroaryl group. In some embodiments, a heteroaryl functional group can be substituted with an aryl or heteroaryl group. In some embodiments, an amino functional group can be substituted with a NH(alkyl) group.

As used herein, the expression “conservative amino acid substitution” refers to a substitution of functionally equivalent or similar amino acids. A conservative amino acid substitution in a peptide brings about a static change to the amino acid sequence of the peptide. For example, one or two or more amino acids having similar polarity act functionally equivalent to each other and bring about a static change in the amino acid sequence of the peptide. In general, a substitution within a certain group may be considered conservative regarding structure and function. However, as is clear to a person having ordinary skill in the art, the role played by a defined amino acid residue may be determined by its implication in the three-dimensional structure of the molecule containing the amino acid. For example, a cysteine residue in an oxidized-type (disulfide) form may have a lower polarity than that of a reduced-type (thiol) form. The long aliphatic part of the arginine side chain may constitute structurally and functionally important features. Furthermore, the side chain (tryptophan, tyrosine, phenylalanine) including an aromatic ring may contribute to ion-aromatic interaction or cation-pi interaction. In such a case, even if the amino acids having these side chains are substituted for amino acids belonging to the acidic or non-polar groups, they may be structurally and functionally conservative. There is a possibility that residues such as proline, glycine, cysteine (disulfide foam) have a direct effect on the three-dimensional structure of the main chain and often may not be substituted without structural distortion.

Conservative amino acid substitution, as shown below, includes specific substitution based on the similarity of side chains (for example, substitutions are described in Lehninger, Biochemistry, Revised 2nd Edition, published in 1975, pp. 73 to 75: L. Lehninger, Biochemistry, 2nd edition, pp. 73 to 75, Worth Publisher, New York (1975)), incorporated herein by reference, and typical substitution.

Hydrophobic amino acids include amino acids that exhibit hydrophobicity, including alanine (also referred to as “Ala” or simply “A”), glycine (also referred to as “Gly” or simply “G”), valine (also referred to as “Val” or simply “V”), leucine (also referred to as “Leu” or simply “L”), isoleucine (also referred to as “Ile” or simply “I”), proline (also referred to as “Pro” or simply “P”), phenylalanine (also referred to as “Phe” or simply “F”), tryptophan (also referred to as Trp” or simply “W”), tyrosine (also referred to as “Tyr” or simply “Y”), and methionine (also referred to as “Met” or simply “M”).

Exemplary hydrophobic amino acids may be further divided into the following groups:
Aliphatic amino acids: Amino acids having a fatty acid or hydrogen in the side chain, including e.g., Ala, Gly, Val, Ile, and Leu.
Aliphatic/branched-chain amino acids: Amino acids having a branched fatty acid in the side chain, including e.g., Val, Ile, and Leu.
Aromatic amino acids: Amino acids having an aromatic ring in the side chain, including e.g., Trp, Tyr, and Phe.

In some embodiments, a hydrophobic amino acid has 4 or more carbon atoms in a side chain (a linear, branched, or cyclic carbon side chain), e.g., Leu, Hcit, Cbg, Chg, or Cba, each of which is optionally N-methylated. In some embodiments, a hydrophobic amino acid has 4-5, 4-6 or 4-7 carbon atoms in a side chain.

Hydrophilic amino acids include amino acids that exhibit hydrophilicity, including e.g., serine (also referred to as “Ser” or simply “S”), threonine (also referred to as “Thr” or simply “T”), cysteine (also referred to as “Cys” or simply “C”), asparagine (also referred to as “Asn” or simply “N”), glutamine (also referred to as “Gln” or simply “Q”), aspartic acid (also referred to as “Asp” or simply “D”), glutamic acid (also referred to as “Glu” or simply “E”), Elysine (also referred to as “Lys” or simply “K”), arginine (also referred to as “Arg” or simply “R”), and histidine (also referred to as “His” or “H”).

Exemplary hydrophilic amino acids may be further divided into the following groups:
Acidic amino acids: Amino acids whose side chains exhibit acidity, including Asp and Glu.
Basic amino acids: Amino acids whose side chains exhibit basicity, including Lys, Arg, and His.
Neutral amino acids: Amino acids whose side chains exhibit neutrality, including Ser, Thr, Asn, Gln, and Cys.

Exemplary hydrophilic amino acids include, for example, N, Q, K, G, S, T, E, Aib, Hcit, Cit, Hgn, KCOpipzaa, Har, Nmm, Ndm, Ala, Hgl, 3Py6NH2, or a variant thereof (including D-amino acid such as da and variations such as Qglucamine, which has gulucamine composition added to the NH₂ terminus of its side chain).

In some embodiments, a peptide described herein comprises an amino acid that affects the direction of the main chain, e.g., Gly and Pro. In some embodiments, a peptide described herein comprises a sulfur-containing amino acid, e.g., Cys and Met. In some embodiments, a peptide described herein comprises an amino acid that comprises an aromatic ring, which can be optionally substituted. Amino acids comprising an aromatic ring include, e.g., F (Phe; phenylalanine), Y (Tyr: tyrosine), W (Trp; tryptophan).

In some embodiments, W or a variant thereof can be W, an amino acid having a heteroatom in the indole ring of W in the side chain, an amino acid in which the hydrogen of NH in the indole ring of W is substituted, or an amino acids having a substituent in the benzene ring of W, or the like.

In some embodiments, F or a variant thereof can be F (phenylalanine), an amino acid wherein (i) the phenyl ring of F is substituted with 1 or 2 substituents each independently selected from —OH, —CN, —C₁₋₃ alkyl, such as —CH₃: (ii) a 6-membered heteroaryl ring optionally substituted by 1 or 2 substituents each independently selected from —OH, —CN, —C₁₋₃ alkyl (such as —CH₃); or (iii-1) having a heteroatom in the phenyl ring of F in the side chain; (iii-2) a derivative amino acid of F in which a 6-membered heteroaryl ring in the side chain is substituted; or the like. In some aspect, F or a variant thereof is optionally N-methylated.

In some embodiments, W, Y or a variant thereof can be W, Y, an amino acid having either a 6-membered aryl or heteroaryl, or a 9- or 10-membered bi-cyclic aryl or heteroaryl linked to the alpha-carbon through a carbon (e.g., a methylene group). In some embodiments, the 6-, 9-, and 10-membered heteroaryl has one heteroatom (e.g., N), and wherein the 6-, 9-, and 10-membered aryl or heteroaryl is optionally substituted with 1 or 2 substituents independently selected from -methyl, -ethyl, —Cl, and —F. In certain embodiments, W or Y or a variant thereof is W1Me, W1Me7Cl, or F23dMe, Nal1, Nal2, W1Et, Nal21N, 3Bzf, 3Bzt, Nal15N, Nal14N, Nal24N, Nal28N, F23dC, or W1Me7N. In some embodiments, a variant of W is W1Me. In some embodiments, a variant of W is W1Me7Cl. In some embodiments, a variant of Y is F23dMe.

Examples of the amino acids include natural protein L-amino acids, unnatural amino acids, and chemically synthesized compounds having properties known in the art as characteristics of an amino acid. Examples of the unnatural amino acids include, but not limited to, α,α-disubstituted amino acids (such as α-methylalanine), N-alkyl-α-amino acids, D-amino acids, β-amino acids, and α-hydroxy acids, each having a backbone structure different from that of natural amino acids; amino acids (such as norleucine and homohistidine) having a side-chain structure different from that of natural amino acids; amino acids (such as “homo” amino acids, homophenylalanine, and homohistidine) having extra methylene in the side chain thereof; and amino acids (such as cysteic acid) obtained by substituting a carboxylic acid functional amino group in the side chain thereof by a sulfonic acid group.

In some embodiments, an amino acid described herein is N-alkylated. In some embodiments, an amino acid described herein is not N-alkylated (e.g., an amino acid with —H on the alpha-amino group). In certain embodiments, such amino acid is A, E, N, K, Qglucamine, KCOpipzaa, Q, Hse, Cit, Hcit, KAc, DapAc, OrnAc, T, alT, Aib, or 3Py6NH2, more preferably, V, Qglucamine, Cit, Hcit, K, or 3Py6NH2.

The peptides described herein can comprise one or more unnatural amino acids. Unnatural amino acids include, but are not limited to, (1) amino acids corresponding to an amino acid residue on a polypeptide subjected to modification after expression (ex. phosphorylated tyrosine, acetylated lysine, or farnesylated cysteine), (2) amino acids that cannot be used in expression on a ribosome but occur naturally, and (3) artificial amino acids that do not occur naturally (unnatural amino acids). Non-limiting examples of unnatural amino acids include: p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl) alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, Boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, and azido-lysine (AzK). In some embodiments, the unnatural amino acid is an unnatural analogue of a tyrosine amino acid; an unnatural analogue of a glutamine amino acid; an unnatural analogue of a phenylalanine amino acid; an unnatural analogue of an alanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid; or a combination thereof. In some embodiments, the unnatural amino acid is an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α, α-disubstituted amino acid; a β-amino acid; a cyclic amino acid other than proline or histidine, or an aromatic amino acid other than phenylalanine, tyrosine or tryptophan.

Unnatural amino acids include, for example, N-alkyl amino acids in which a natural amino acid described above is N-alkylated, e.g., those modified with lower alkyl groups (for example, of C1 to C5, C1 to C3, and C1) in which the nitrogen forming a peptide bond is branched or not branched. Exemplary N-alkyl amino acids include, e.g., N-ethyl amino acid, N-butyl amino acid, and N-methyl amino acid. Also included are amino acids to which a functional group is further added to the side chain of a natural amino acid or substituted for another functional group (for example, an amino acid having a substitution or an addition in a part such as an arylene group, an alkylene group, or the like of the side chain; an amino acid wherein the arylene group or the alkyl group of the side chain has an increased C-number; an amino acid having a substitution in the aromatic ring of the side chain; a heterocyclic or condensed cyclic amino acid; or the like). Exemplary N-alkyl amino acids further include, e.g., N-alkyllysine and N-methyllysine. Exemplary N-alkyl amino acids further include, e.g., N-methyllysine in which an albumin binder is bound.

In a non-limiting manner, unnatural amino acids include, but are not limited to N-methyl amino acids, da, kCOpipzaa, dahp, df3CON, 4Py, W7N, QPh, alT, W1Me, Cbg, Chg, Cba, Hgl, Hgn, Nmm, Ndm, Hcit, Qglucamine, Hph, W1Me7N, W1Me7Cl, 3Py6NH2, Cit, F23dMe, Har, bA, Kac, dkAc, MeF, Me3Py, MeHph, MeF3CN, MeF3H, MeE, MeN, MeF4C, Nal1, Nal2, W1Et, Nal21N, 3Bzf, 3Bzt, al15N, Nal14N, Nal24N, Nal28N, F23dMe, F23dC, W1Me7N, W1Me7Cl, Hse, DapAc, OrnAc, Alb, and the like. Note that D-amino acids such as da may be classified as D-amino acids, but they may also be classified according to the properties of their side chains, and N-methyl amino acids may be classified as N-alkyl amino acids and may also be classified according to the property of the side chain.

In some embodiments, the unnatural amino acids incorporated into the peptides include one or more of: 1) a ketone functional group (as found in para or meta acetyl-phenylalanine) that can be specifically reacted with hydrazines, hydroxylamines and their derivatives (Addition of the keto functional group to the genetic code of Escherichia coli. Wang L, Zhang Z, Brock A, Schultz P G. Proc Natl Acad Sci USA. 2003 Jan. 7; 100 (1): 56-61; Bioorg Med Chem Lett. 2006 Oct. 15; 16 (20): 5356-9. Genetic introduction of a diketone-containing amino acid into proteins. Zeng H, Xie J, Schultz P G), 2) azides (as found in p-azido-phenylalanine) that can be reacted with alkynes via copper catalyzed “click chemistry” or strain promoted (3+2) cycloadditions to form the corresponding triazoles (Addition of p-azido-L-phenylalanine to the genetic code of Escherichia coli. Chin J W, Santoro S W, Martin A B, King D S, Wang L, Schultz P G. J Am Chem Soc. 2002 Aug. 7; 124 (31): 9026-7; Adding amino acids with novel reactivity to the genetic code of Saccharomyces cerevisiae. Deiters A, Cropp T A, Mukherji M, Chin J W, Anderson J C, Schultz P G. J Am Chem Soc. 2003 Oct. 1; 125 (39): 11782-3), or azides that can be reacted with aryl phosphines, via a Staudinger ligation (Selective Staudinger modification of proteins containing p-azidophenylalanine. Tsao M L, Tian F, Schultz P G. Chembiochem. 2005 December; 6 (12): 2147-9), to form the corresponding amides, 3)alkynes that can be reacted with azides to form the corresponding triazole (In vivo incorporation of an alkyne into proteins in Escherichia coli. Deiters A, Schultz P G. Bioorg Med Chem Lett. 2005 Mar. 1; 15 (5): 1521-4), 4) boronic acids (boronates) than can be specifically reacted with compounds containing more than one appropriately spaced hydroxyl group or undergo palladium mediated coupling with halogenated compounds (Angew Chem Int Ed Engl. 2008; 47 (43): 8220-3. A genetically encoded boronate-containing amino acid, Brustad E, Bushey M L, Lee J W, Groff D, Liu W, Schultz P G), and 5) metal chelating amino acids, including those bearing bipyridyls, that can specifically co-ordinate a metal ion (Angew Chem Int Ed Engl. 2007; 46 (48): 9239-42. A genetically encoded bidentate, metal-binding amino acid. Xie J, Liu W, Schultz P G).

The peptide of the present disclosure embraces various derivatives thereof. Examples of the derivatives include derivatives having an amide, ester, or carboxyl group as the C-terminus and/or N-terminus thereof. Additional examples of the derivatives of the peptide include those obtained by modification such as phosphorylation, methylation, acetylation, adenylylation, ADP-ribosylation, or glycosylation and fused protein obtained by fusion with another peptide or protein. These derivatives can be prepared by those skilled in the art in a known manner or a method based thereon.

In some embodiments, the peptide described herein comprises a basic amino acid. Examples of the basic amino acid include arginine, lysine, citrulline, ornithine, creatine, histidine, diaminobutanoic acid, and diaminopropionic acid.

In some embodiments, provided herein is a peptide having 90% or more sequence identity to any of sequences disclosed herein. In some embodiments, the sequence identity is at least 95% or 99%.

In some embodiments, the peptide is bicyclic or polycyclic. In some embodiments, a conjugate described herein comprises a bicyclic peptide. Exemplary bicyclic peptides include the bicyclic targeting peptides of BT5528, BT1718, and BT8009. Exemplary bicyclic peptides are described in US20180200378, U.S. Pat. Nos. 10,441,663, 8,680,022B2, US20180280525, and US20200215199, each of which is hereby incorporated by reference in its entirety. In some cases, when a peptide is cyclized, protease resistance is improved, metabolic stability is improved, and restrictions are also added to conformational change, so that rigidity is increased and membrane permeability and affinity for the target protein is improved.

In some embodiments, the peptide of the present disclosure has a cyclic structure in which a chloroacetylated amino acid and a cysteine residue present in the peptide are bound. In one aspect, the peptide has a cyclic structure in which an N-terminal amino acid and a cysteine residue present in the peptide are bound. In some embodiments, the peptide has a cyclic structure in which an N-terminal amino acid and the thirteenth cysteine residue present in the peptide are bound. In some embodiments, the peptide has a cyclic structure in which a chloroacetylated N-terminal amino acid and the 12th cysteine residue present in the peptide are bound. “Chloroacetylation” may be replaced with “haloacetylation” using another halogen. Furthermore, “acetylation” may be “acylation” using an acyl group other than an acetyl group.

In some embodiments, the peptide is a lasso peptide. Lasso peptides can be synthetic or naturally produced by bacteria, and they possess a distinctive threaded lariat fold that offers a 3D array of functionality for engaging biological targets. This lasso structure can enable beneficial properties such as affinity, stability and potent biological activities. Suitable lasso structure can be designed by algorithms. Exemplary lasso peptides are provided in Hegemann, J. D., et al., Lasso Peptides: An Intriguing Class of Bacterial Natural Products, Acc. Chem. Res., 2015, 48, 1909-1919; Tietz, J. I., et al., A new genome-mining tool redefines the lasso peptide biosynthetic landscape, Nature Chem Bio, 2017, 13, 470-478; DiCaprio, A. J., et al., Enzymatic Reconstitution and Biosynthetic Investigation of the Lasso Peptide Fusilassin, J. Am. Chem. Soc., 2019, 141, 290-297; A1 Toma, R. S., et al., Site-Directed and Global Incorporation of Orthogonal and Isostructural Noncanonical Amino Acids into the Ribosomal Lasso Peptide Capistruin, ChemBioChem, 2015, 16, 503-509.

Further exemplary peptides include BMS-753493, Somatostatins, Octreotide, Octreotate, Lanreotide, Pasireotide, JR-11, L-779,976, BIM-23120, Satoreotide, depreotide, 18F-KYNDRLPLYISNP (SEQ ID NO: 274), CaIX-P1, and FAP-2286.

The peptide of the present disclosure embraces salts thereof. As the salts of the peptide, salts with physiologically acceptable base or acid are used. Examples include addition salts with an inorganic acid (such as hydrochloric acid, hydrobromic acid, hydroiodic acid, sulfuric acid, or phosphoric acid), addition salts with an organic acid (such as p-toluenesulfonic acid, methanesulfonic acid, oxalic acid, p-bromophenylsulfonic acid, carboxylic acid, succinic acid, citric acid, benzoic acid, or acetic acid), inorganic bases (such as ammonium hydroxide, alkali or alkaline earth metal hydroxide, carbonate, or bicarbonate), and an amino acid.

The peptide of the present disclosure can be prepared by a known peptide preparation method, for example, chemical synthesis method such as liquid-phase method, solid-phase method, or hybrid method using a liquid-phase method and a solid-phase method in combination; or gene recombination method.

In solid-phase method, an esterification reaction can be performed, for example, between the hydroxyl group of a hydroxyl-containing resin and the carboxyl group of a first amino acid (usually, C-terminal amino acid of an intended peptide) having an α-amino group protected with a protecting group. As the esterifying catalyst, a dehydration condensation agent such as 1-mesitylenesulfonyl-3-nitro-1,2,4-triazole (MSNT), dicyclohexylcarbodiimide (DCC), and diisopropylcarbodiimide (DIPCDI) may be used. Next, the protecting group of the α-amino group of the first amino acid is eliminated and at the same time, a second amino acid having all the functional groups protected except the main chain carboxyl group is added to activate the carboxyl group and bind the first and second amino acids to each other. Then, the α-amino group of the second amino acid is deprotected, a third amino acid having all the functional groups protected except the main chain carboxyl group is added, and the carboxyl group is activated to bind the second and third amino acids to each other. The above-described reactions are repeated to synthesize a peptide having an intended length. Then, all the functional groups are deprotected. Examples of the resin for solid-phase synthesis include Merrifield resin, MBHA resin, CI-Trt resin, SASRIN resin, Wang resin, Rink amide resin, HMFS resin, Amino-PEGA resin (Merck), and HMPA-PEGA resin (Merck). These resins may be provided for use after washed with a solvent (dimethylformamide (DMF), 2-propanol, methylene chloride, or the like). A peptide chain can be cleaved from the resin by treating it with an acid such as TFA or hydrogen fluoride (HF).

Examples of the protecting group of the α-amino group include a benzyloxycarbonyl (Cbz or Z) group, a tert-butoxycarbonyl (Boc) group, a fluorenylmethoxycarbonyl (Fmoc) group, a benzyl group, an allyl group, and an allyloxycarbonyl (Alloc) group. The Cbz group can be deprotected using hydrofluoric acid, hydrogenation, or the like; the Boc group can be deprotected using trifluoroacetic acid (TFA); and the Fmoc group can be deprotected by the treatment with piperidine. For protection of the α-carboxyl group, a methyl ester, an ethyl ester, a benzyl ester, a tert-butyl ester, a cyclohexyl ester, or the like can be used. As other functional groups of an amino acid, the hydroxyl group of serine or threonine can be protected with a benzyl group or a tert-butyl group and the hydroxyl group of tyrosine can be protected with a 2-bromobenzyloxycarbonyl group or a tert-butyl group. The amino group of a lysine side chain or the carboxyl group of glutamic acid or aspartic acid can be protected in a manner similar to the α-amino group or a-carboxyl group.

The carboxyl group can be activated with a condensation agent. Examples of the condensation agent include dicyclohexylcarbodiimide (DCC), diisopropylcarbodiimide (DIPCDI), 1-ethyl-3-(3-dimethylaminopropyl) carbodiimide (EDC or WSC), (1H-benzotriazol-1-yloxy)tris(dimethylamino)phosphonium hexafluorophosphate (BOP), and 1-[bis(dimethylamino)methyl]-1H-benzotriazolium-3-oxide hexafluorophosphate (HBTU).

Peptide preparation based on the recombinant method (translation and synthesis system) can be performed using a nucleic acid encoding the peptide of the present disclosure. The nucleic acid encoding the peptide can be either DNA or RNA. The nucleic acid encoding the peptide can be prepared in a known method. For example, it can be synthesized using an automated synthesizer. The DNA thus obtained may have therein a restriction enzyme recognition site for inserting it into a vector or may have therein a base sequence that encodes an amino acid sequence for cleavage of the resulting peptide chain by an enzyme. The peptide obtained may be converted from a free peptide to a salt thereof or from a salt thereof to a free peptide by a known method or a method based thereon.

In order to suppress decomposition by a host-derived protease, a chimera protein expression method that expresses the intended peptide as a chimera peptide with another peptide can be used. In this case, as the nucleic acid, a nucleic acid encoding the intended peptide and a peptide that binds thereto is used. Then, an expression vector is prepared using the nucleic acid encoding the peptide of the present disclosure. The nucleic acid can be inserted into downstream of a promoter of an expression vector as it is, or after digestion with a restriction enzyme or addition of a linker. Examples of the vector include Escherichia coli-derived plasm ids (such as pBR322, pBR325, pUC12, pUC13, pUC18, pUC19, pUC118, and pBluescript II), Bacillus subtilis-derived plasmids (such as pUB110, pTP5, pC1912, pTP4, pE194, and pC194), yeast-derived plasmids (such as pSH19, pSH15, YEp, YRp, Ylp, and YAC), bacteriophages (such as e phage and M13 phage), viruses (retrovirus, vaccinia virus, adenovirus, adeno-associated virus (AAV), cauliflower mosaic virus, tobacco mosaic virus, and baculovirus), and cosmids. The promoter can be selected as needed, depending on the type of the host. When the host is an animal cell, for example, a SV40 (simian virus 40)-derived promoter or a CMV (cytomegalovirus)-derived promoter can be used. When the host is Escherichia coli, a trp promoter, a T7 promoter, a lac promoter, or the like can be used. The expression vector may incorporate therein a nucleic acid encoding a DNA replication origin (ori), a selection marker (antibiotic resistance, nutrition requirement, or the like), an enhancer, a splicing signal, a polyadenylation signal, a tag (FLAG, HA, GST, GFP, or the like), or the like.

Next, an appropriate host cell is then transformed using the above-described vector. The host can be selected as needed based on the relation with a vector and for example, Escherichia coli, Bacillus subtilis, Bacillus bacteria), yeasts, insects or inset cells, and animal cells can be used. Examples of the animal cells include HEK293T cells, CHO cells, COS cells, myeloma cells, HeLa cells, and Vero cells. Transformation can be performed in a known manner such as lipofection, calcium phosphate method, electroporation, microinjection, or particle gun technology, depending on the type of hosts. By culturing the transformant in a conventional manner, an intended peptide is expressed. The peptide from the cultured product of the transformant can be purified in the following manner. Cultured cells collected and then suspended in an appropriate buffer are destructed by ultrasonic treatment, freezing and thawing method, or the like and the resulting destructed product centrifuged or filtered to obtain a crude extract. When the peptide is secreted in the culture fluid, a supernatant is collected. Purification of the crude extract or culture supernatant can also be performed by a known method or a method based thereon (for example, salting-out, dialysis, ultrafiltration, gel filtration, SDS-PAGE, ion exchange chromatography, affinity chromatography, or reverse-phase high-performance liquid chromatography).

The system for translation and synthesis may be a cell-free translation system. The cell-free translation system may include, for example, a ribosome protein, aminoacyl tRNA synthetase (ARS), ribosome RNA, an amino acid, rRNA, GTP, ATP, a translation initiation factor (IF), an elongation factor (EF), a release factor (RF), a ribosome regeneration factor (RRF), and other factors necessary for translation. An Escherichia coli extract or wheat bran extract may be added in order to increase the expression efficiency. Further, a rabbit erythrocyte extract or insect cell extract may be added. Continuous energy supply to a system containing the above by dialysis can enable production of several hundred μg to several mg/mL of a protein. The system may contain RNA polymerase for carrying out transcription from DNA at the same time. As a commercially available cell-free translation system, an Escherichia-coli derived system such as “RTS-100™” of Roche Diagnostics Corporation or PURESYSTEM™ of PGI Corporation or a system using wheat germ extract such as that of ZOEGENE Corporation or Cell-free Science may be used. By using the cell-free translation system, a high-purity peptide can be obtained without purifying the expression product.

In the cell-free translation system, an artificial aminoacyl tRNA obtained by linking (acylating) a desired amino acid or hydroxy acid to tRNA can be used instead of an aminoacyl tRNA synthesized by a native aminoacyl tRNA synthetase. Such an aminoacyl tRNA can be synthesized using an artificial ribozyme. Examples of such a ribozyme include flexizymes (H. Murakami, A. Ohta, H. Ashigai, H. Suga (2006) Nature Methods 3, 357-359 “The flexizyme system: a highly flexible tRNA aminoacylation tool for the synthesis of nonnatural peptides”; WO2007/066627; and the like). Flexizyme is also known as, as well as flexizyme (Fx) in original form, dinitrobenzyl flexizyme (dFx), enhanced flexizyme (eFx), or aminoflexizyme (aFx), each obtained by modifying the original one. By using a tRNA having a desired amino acid or hydroxy acid linked thereto and prepared using flexizyme, a desired codon can be translated while associating the codon with the desired amino acid or hydroxy acid. As the desired amino acid, a non-canonical amino acid may be used. For example, a non-natural amino acid necessary for the above-described cyclization can be introduced into the peptide by this method. For example, an in vitro system can be employed wherein nonsense mutations are suppressed using chemically aminoacylated suppressor tRNAs. Transcription and translation of plasmids containing nonsense mutations can be carried out in a cell-free system comprising e.g., an E. coli S30 extract and commercially available enzymes and other reagents. Peptides can be purified by chromatography. As another example, translation can be carried out in Xenopus oocytes by microinjection of mutated mRNA and chemically aminoacylated suppressor tRNAs. For yet another example, E. coli cells can be cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid can be incorporated into the peptide in place of its natural counterpart. Naturally occurring amino acid residues can also be converted to non-naturally occurring species by in vitro chemical modification. Chemical modification can be combined with site-directed mutagenesis to further expand the range of substitutions.

Peptides described herein can also be produced by chemical synthesis (e.g., by the methods described in Solid Phase Peptide Synthesis, 2nd Edition, The Pierce Chemical Co., Rockford, Ill. (1984)). Modifications to the protein can also be produced by chemical synthesis.

### Linker

A conjugate described herein can comprise one or more linkers. In some embodiments, the linker covalently attaches the peptide with the metal chelator. In some embodiments, the peptide attaches directly to the metal chelator without a linker. In some embodiments, the linker covalently attaches the peptide with the covalently bound radionuclide. In some embodiments, the peptide attaches directly to the covalently bound radionuclide without a linker. In some embodiments, the covalently bonded radioisotope is attached to the radiolabeled conjugate through a chemical linker. In some embodiments, a radiopharmaceutical conjugate described herein can comprise one or more linkers connecting one or more covalent radionuclides to the peptide. The one or more linkers can each independently bind a covalent radioisotope. In some embodiments, the covalent radioisotope is selected from a radioisotope in Table 7 labeled “covalent”. In some embodiments, the covalent radioisotope is selected from fluorine-18 (¹⁸F), iodine-131 (¹³¹I), iosine-123 (¹²³I), iodine-124 (¹²⁴I), iodine-125 (¹²⁵I), or astatine-211 (²¹¹At). In some embodiments, the covalent radioisotope is ¹³¹I. In some embodiments, the covalent radioisotope is ¹²⁴I. In some embodiments, the covalent radioisotope is ¹²⁵I. In some embodiments, the covalent radioisotope is ²¹¹At.

In some embodiments, the present disclosure describes linkers that function as a spacer. A linker can comprise a number of intervening atoms (on a linear chain, excluding pendant groups or substituents) between the metal chelator and the binding peptide thereby creating a distance between the metal chelator and the binding peptide. In some embodiments, a linker comprises 10-100 intervening atoms between the metal chelator and the binding peptide. In some embodiments, a linker comprises 2-60 intervening atoms between the metal chelator and the binding peptide. In some embodiments, a linker comprises 2 to 20, 2 to 50, 5 to 15, 5 to 25, 10 to 40, 30 to 60, or 10 to 20 intervening atoms between the metal chelator and the binding peptide. In some embodiments, a linker comprises 3 to 30 intervening atoms between the metal chelator and the binding peptide. In some embodiments, a linker comprises 5 to 25 intervening atoms between the metal chelator and the binding peptide. In some embodiments, a linker comprises 6 to 18 intervening atoms between the metal chelator and the binding peptide. In some embodiments, a linker comprises 10 to 20 intervening atoms between the metal chelator and the binding peptide. The intervening atoms can comprise 1 or more carbons, and optionally one or more heteroatoms such as O and N. In some embodiments, the intervening atoms comprise 2 to 20, 2 to 50, 5 to 15, 5 to 25, 10 to 40, 30 to 60, or 10 to 20 carbons. In some embodiments, the intervening atoms comprise 0, 1, 2, 3, 4, 5, or 6 nitrogen. In some embodiments, the intervening atoms comprise 0, 1, 2, 3, 4, 5, 6, 7 or 8 oxygen. In some embodiments, the intervening atoms comprise 1 to 6 nitrogen and 0 to 4 oxygen.

A linker can comprise one or more amino acid residues. In some embodiments, the linker comprises 1 to 3, 1 to 5, 1 to 10, 5 to 10, or 5 to 20 amino acid residues. In some embodiments, the linker comprises 1, 2, 3, 4, 5, 6, 7, 8, 9, or 10 amino acid residues. In some embodiments, the linker comprises 1 to 5 amino acid residues. For example, the linker can comprise one or more lysine (K) residues such as K, KK, or KKK sequences. In some embodiments, the linker comprises a lysine or a derivative thereof. In some embodiments, the linker comprises a lysine. In some embodiments, one or more amino acids of the linker are unnatural amino acids. In some embodiments, the linker comprises a lysine residue, an alanine residue, or both. In some embodiments, the linker comprises a lysine residue. In some embodiments, the linker comprises an alanine residue. In some embodiments, the linker comprises an amino acid residue selected from a lysine residue, an alanine residue, a glycine residue, a d-phenylalanine and a phenylalanine residue. In some embodiments, the linker comprises a second amino acid residue selected from a lysine residue, an alanine residue, a glycine residue, a d-phenylalanine and a phenylalanine residue. In some embodiments, the linker comprises a third (or more) amino acid residue selected from a lysine residue, an alanine residue, a glycine residue, a d-phenylalanine and a phenylalanine residue.

A herein-described linker can attach to the N-terminus of the peptide, the C-terminus of the peptide, or a non-terminal amino acid of the peptide, or it can attach to the peptide through a combination of the above. In some embodiments, the linker is attached to the peptide via its N-terminus. In some embodiments, the linker is attached to the peptide via a cysteine residue at the C-terminus. In some embodiments, the linker is attached to the peptide via a cysteine residue at the N-terminus. In some embodiments, the linker is attached to the peptide via its C-terminus. In some embodiments, the linker is attached to the peptide via a non-terminal amino acid. The linker can be bonded to the peptide, the metal chelator, or both, for example, through a chemically reactive group. Exemplary chemically reactive groups include, but are not limited to, a free amino, imino, hydroxyl, thiol or carboxyl group (e.g., to the N- or C-terminus, to the epsilon amino group of one or more lysine residues, the free carboxylic acid group of one or more glutamic acid or aspartic acid residues, or to the sulfhydryl group of one or more cysteinyl residues). The site to which the linker is bound to the peptide can be a natural or unnatural amino acid of the peptide and/or it can be introduced into the peptide, e.g., by DNA recombinant technology (e.g., by introducing a cysteine or protease cleavage site in the amino acid sequence) or by protein biochemistry (e.g., reduction, pH adjustment or proteolysis). Exemplary methods for attaching the linker includes carbodiimide reaction, reactions using bifunctional agents such as dialdehydes or imidoesters, Schiff base reaction, Suzuki-Miyaura cross-coupling reactions, Isothiocyanates as coupling agents, and click chemistry.

The linker can have a prescribed length thereby linking the metal chelator (and optionally radionuclide) and the peptide while allowing an appropriate distance therebetween. In some embodiments, the linker has 1 to 100 atoms, 1 to 60 atoms, 1 to 30 atoms, 1 to 15 atoms, 1 to 10 atoms, 1 to 5, or 2 to 20 atoms in length. In some embodiments, the linker has 1 to 10 atoms in length.

The linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences (e.g., EAAAK (SEQ ID NO: 278)), proline-rich sequences, and regions rich in double and/or triple bonds.

In some embodiments, a linker may be further added to the (cyclic) peptide. Examples of the linker include the foregoing amino acid linker (peptide linker), a chemical linker, a fatty acid linker, a nucleic acid linker, a sugar chain linker, or the like, or it may be a complex, for example, a chemical linker, a peptide linker, or the like. Examples of the chemical linker include a PEG (polyethylene glycol) linker. For example, the PEG linker may comprise between 1 to 24 ethylene glycol units. Furthermore, the linker may be a fatty acid linker containing a divalent chemical moiety derived from a fatty acid. In some embodiments, the linker comprises at least one amino acid, and, for example, a glycine-rich peptide such as a peptide having a sequence [Gly-Gly-Gly-Gly-Ser] n (in the formula, n is 1, 2, 3, 4, 5, or 6) (SEQ ID NO: 275).

The linker may be added at any position. For example, it may be bound to Cys positioned on the C-terminal side or may be bound to an amino acid comprised in the cyclic peptide. In some instances, it is bound to Cys or variant thereof positioned on the C-terminal side. In some instances, a linker can be added to the —COOH on the Cys residue. It may be possible to add one to several amino acids to the C-terminus of such Cys residue and then the linker can be added to its terminus; for example, Gly is added to the C-terminus of Cys within the cyclic structure peptide, then the —COOH of the Gly is bound to a linker. In some instances, a linker is added to the side chain on amino acid, e.g., Lys, within the cyclic peptide. For example, in some instances, a linker can be added to the side chain of Lys at X3, X5, X8 or X10.

The linker can be cleavable, e.g., under physiological conditions, e.g., under intracellular conditions, such that cleavage of the linker releases the chelator and radionuclide in the intracellular environment. The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin. In other embodiments, the linker is not cleavable. In some embodiments, the linker is pH-sensitive, i.e., sensitive to hydrolysis at certain pH values. For example, the pH-sensitive linker can be hydrolyzable under acidic conditions. For example, a linker can be an acid-labile linker that is hydrolyzable in the lysosome (e.g., a hydrazone, semicarbazone, thiosemicarbazone, cis-aconitic amide, orthoester, acetal, ketal, or the like). Such linkers can be relatively stable under neutral pH conditions, such as those in the blood, but are unstable at below pH 5.5 or 5.0, the approximate pH of the lysosome. In some embodiments, the hydrolyzable linker is a thioether linker.

In some embodiments, the linker comprises an amino acid sequence, such as a combination of amino acid sequence and a flexible and/or rigid region, as exemplified in Table B6-1, shown in the “Linker” column. For example, PDC_EphA2-00010011-C003 includes a linker comprising an amino acid residue, bA-dk. In another example, PDC_EphA2-00001417-C004 includes a linker comprising a combination of amino acid residues and PEG: kA-dk-(PEG8c-PEG2c).

In some embodiments, the linker comprises one or more of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, the linker comprises substituted or unsubstituted C₁-C₃₀ alkylene. In some embodiments, the linker comprises polyethylene glycol such as (—CH₂—CH₂—O—)₁₋₁₀. In some embodiments, the linker comprises a structure selected from:

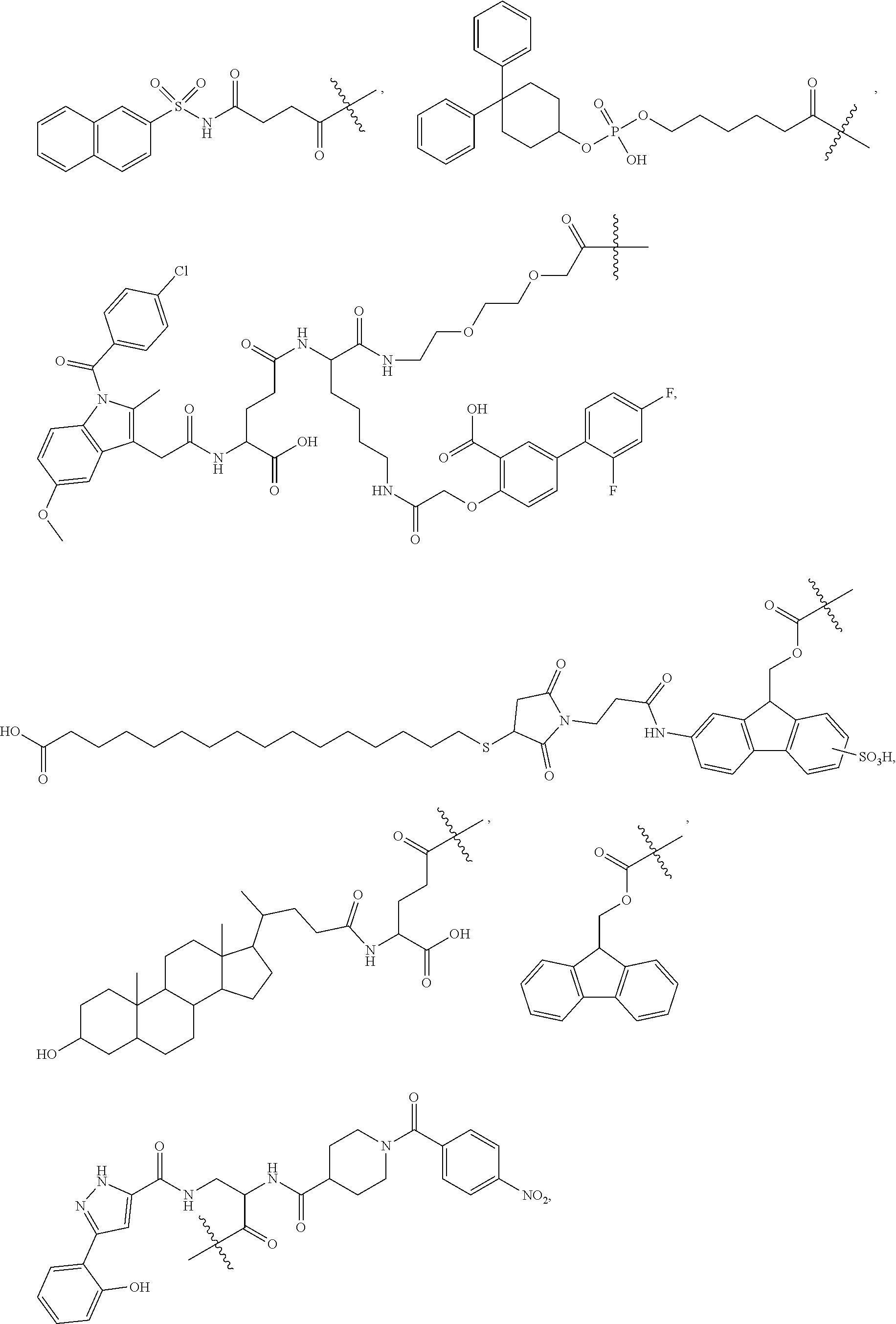

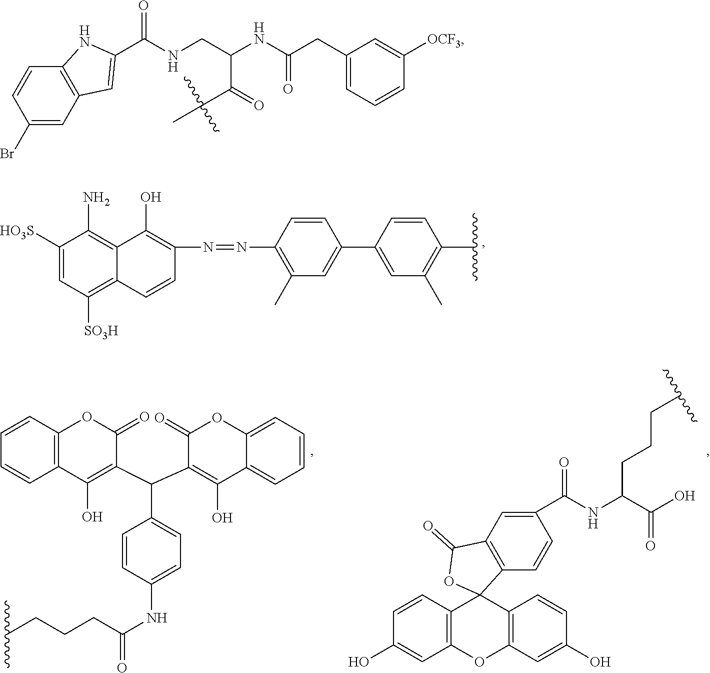 [SMILES: *C(=O)C(CNC(=O)c1cc2cc(Br)ccc2[nH]1)NC(=O)Cc1cccc(OC(F)(F)F)c1 ; *c1ccc(-c2ccc(N=Nc3ccc4c(S(=O)(=O)O)cc(S(=O)(=O)O)c(N)c4c3O)c(C)c2)cc1C ; *CCCC(=O)Nc1ccc(C(c2c(O)c3ccccc3oc2=O)c2c(O)c3ccccc3oc2=O)cc1 ; *CCCC(NC(=O)c1ccc2c(c1)C(=O)OC21c2ccc(O)cc2Oc2cc(O)ccc21)C(=O)O]

and structures derived from any one thereof.

In some embodiments, the linker comprises a click chemistry residue. In some embodiments, the linker is attached to the peptide, to the metal chelator, or both via click chemistry, thereby forming a click chemistry residue. For example, the peptide can comprise an azide group (at N- or C-terminus or at a non-terminal amino acid) that reacts with an alkyne moiety of the linker. For another example, the peptide can comprise an alkyne group (at N- or C-terminus or at a non-terminal amino acid) that reacts with an azide of the linker. The metal chelator and the linker can be attached similarly. In some embodiments, the linker comprises an azide moiety, an alkyne moiety, or both. In some embodiments, the linker comprises a triazole. In some embodiments, the click chemistry residue is

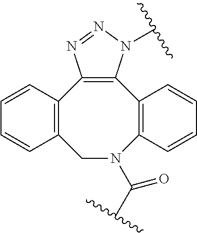 [SMILES: *C(=O)N1Cc2ccccc2-c2nnn(*)c2-c2ccccc21]

(DBCO-azide residue),

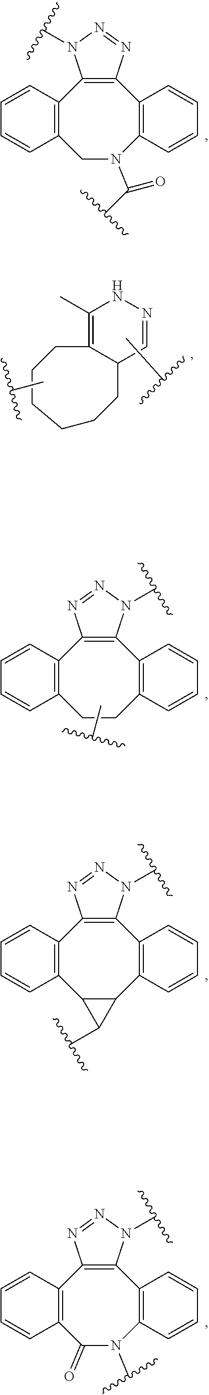

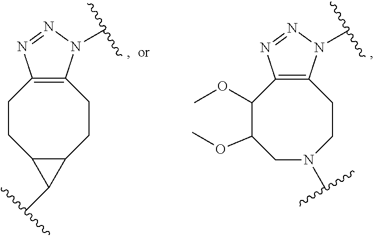 [SMILES: *C1C2CCc3nnn(*)c3CCC12 ; *N1CCc2c(nnn2*)C(OC)C(OC)C1]

In some embodiments, the click chemistry residue is a DIBO-azide residue, BARAC-azide residue, DBCO-azide residue, DIFO-azide residue, COMBO-azide residue, BCN-azide residue, or DIMAC-azide residue. In some embodiments, the linker comprises a residue of nitrone dipole cycloaddition. In some embodiments, the linker comprises a residue of tetrazine ligation. In some embodiments, the linker comprises a residue of quadricyclane ligation. Exemplary groups of click chemistry residue are shown in Hein at al., “Click Chemistry, A Powerful Tool for Pharmaceutical Sciences,” Pharmaceutical Research volume 25, pages 2216-2230 (2008); Thirumurugan et al, “Click Chemistry for Drug Development and Diverse Chemical-Biology Applications,” Chem. Rev. 2013, 113, 7, 4905-4979; US20160107999A1; U.S. Pat. No. 10,266,502B2; and US20190204330A1, each of which is incorporated by reference in its entirety.

In some embodiments, a linker described herein comprises two or more motifs. In some embodiments, one or more of the motifs are connected via click chemistry such that they can be clicked in/out of the linker. Each of the motifs in a linker can have independent functions. For example, a linker can comprise a motif that functions to adjust plasma half-life and/or a motif that functions as a spacer between the peptide and metal chelator or covalebound radionuclide.

A linker described herein can comprise a residualizing agent or a non-residualizing agent. A radionuclide can be attached to a peptide or a linker through a residualizing agent or a non-residualizing agent. In some embodiments, the radionuclide is covalently attached to the peptide or the linker through a residualizing agent. In some embodiments, the residualizing agent is SGMIB or SIPC. In some embodiments, the radionuclide is covalently attached to the peptide or the linker through a residualizing agent. In some embodiments, the non-residualizing agent is N-succinimidyl-4-iodobenzoate (PIB). In some embodiments, a radionuclide is covalently bound to the residualizing agent or the non-residualizing agent. In some embodiments, the radionuclide is covalently bound to the residualizing agent. Procedures and methods for synthesis of covalently bound residualizing agents are described in U.S. Pat. No. 9,839,704 which is herein incorporated by reference in its entirety.

In some embodiments, the residualizing agent is a tetrapeptide IMP-R4. IMP-R4 can be represented as MCC-Lys(MCC)-Lys(Z)-d-Tyr-d-Lys(Z)-OH (SEQ ID NO: 415), where MCC is 4-(N-maleimidomethyl)-cyclohexane-1-carbonyl and Z is 1-((4-thiocarbonylamino)benzyl)-DTPA. In some embodiments, the radionuclide ¹³¹I is linked to linker or peptide via ¹³¹I-IMP-R. In some embodiments, the residualizing agent is a tetrapeptide IMP-R3. In some embodiments, the residualizing agent is a tetrapeptide IMP-R5. In some embodiments, the residualizing agent is a tetrapeptide IMP-R6. In some embodiments, the residualizing agent is a tetrapeptide IMP-R7. In some embodiments, the residualizing agent is a tetrapeptide IMP-R8. Exemplary residualizing and non-residualizing agents are further illustrated in Stein R, et al. Improved iodine radiolabels for monoclonal antibody therapy, Cancer Res. 2003; 63:111-118; Reist C J, et al. Radioiodination of internalizing monoclonal antibodies using N-succinimidyl-5-iodo-3-pyridinecarboxylate, Cancer Res. 1996; 56:4970-4977; Ali S A, et al. Improving the tumor retention of radioiodinated antibody: aryl carbohydrate adducts. Cancer Res. 1990; 50 (suppl): 783s-788s; and Serengulam V. Govindan, et al., Clinical-Scale Radiolabeling of a Humanized Anticarcinoembryonic Antigen Monoclonal Antibody, hMN-14, with Residualizing 131I for Use in Radioimmunotherapy, Journal of Nuclear Medicine January 2005, 46 (1) 153-159.

In some embodiments, the linker has a structure of

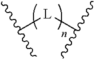

wherein each L is independently —O—, —NR^{L}—, —N(R^{L})₂⁺—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, —(C1-C30 alkylene)-O—, —O—(C₁-C₃₀ alkylene)-, —(C₁-C₃₀ alkylene)-NR^{L}—, —NR^{L}—(C₁-C₃₀ alkylene)-, —(C₁-C₃₀ alkylene)-N(R^{L})₂⁺—, —N(R^{L})₂⁺—(C₁-C₃₀ alkylene)-, or a click chemistry residue; and
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
n is 1 to 20 (e.g., 1, 2, 3, 4, 5, 6, 7, 8, 9, 10, 11, 12, 13, 14, 15, 16, 17, 18, 19, or 20). In some embodiments, one of L optionally comprises a residualizing agent or non-residualizing agent. In some embodiments of Formula (II-1), n is 0. When n is 0, the linker of Formula (II-1) is a bond.

In some embodiments, the linker has a structure of

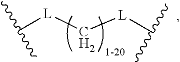

wherein each L is independently —O—, —NR^{L}—, —N(R^{L})₂⁺—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—.

In some embodiments, a linker disclosed herein is optionally substituted C₁-C₃₀ alkylene or C₁-C₃₀ heteroalkylene. In some embodiments, the linker is optionally substituted C₁-C₁₀ alkylene or C₁-C₁₀ heteroalkylene. In some embodiments, the linker is optionally substituted C₁-C₈ alkylene or C₁-C₈ heteroalkylene. In some embodiments, the linker is optionally substituted C₁-C₆ alkylene or C₁-C₆ heteroalkylene. In some embodiments, the linker is optionally substituted C₁-C₆ alkylene or C₁-C₆ heteroalkylene. In some embodiments, the linker is optionally substituted C₁-C₄ alkylene or C₁-C₄ heteroalkylene. In some embodiments, the linker is optionally substituted C₁-C₆ heteroalkylene (e.g.,

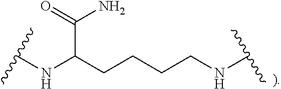 [SMILES: *NCCCCC(N*)C(N)=O]

In some embodiments, the linker is an optionally substituted C₁-C₂₀ heteroalkylene, comprising 1-20 heteroatoms selected from O, S, and N. In some embodiments, the linker is a heteroalkylene comprising 1-4 heteroatoms selected from N, S and O. In some embodiments, the linker is a heteroalkylene comprising 1-3 heteroatoms selected from N and O. In some embodiments, the linker is an optionally substituted C₁-C₁₅ heteroalkylene, comprising 1-15 heteroatoms selected from O, S, and N. In some embodiments, the linker is a heteroalkylene, wherein the heteroalkylene comprises one or more —CH₂—CH₂—O— units. In some embodiments, the linker is a heteroalkylene, wherein the heteroalkylene comprises 1-15 —CH₂—CH₂—O— units. In some embodiments, the linker is a heteroalkylene, wherein the heteroalkylene comprises 1-5 —CH₂—CH₂—O— units. In some embodiments, the linker is a heteroalkylene, wherein the heteroalkylene comprises 5-20 —CH₂—CH₂—O— units. In some embodiments, the —CH₂—CH₂—O— units are contiguous. In some embodiments, the linker is optionally substituted with one or more substituents selected from halogen, —CN, oxo, —OH, —OC₁-C₆alkyl, SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NR^{b}C(═NR^{b})NR^{c}R^{d}, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, and C₁-C₆heteroalkyl. In some embodiments, the linker is optionally substituted with one or more substituents selected from halogen, —CN, —OH, oxo, —OC₁-C₆alkyl, —NH₂, —NHC₁-C₆alkyl, —N(C₁-C₆alkyl)₂, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, and C₁-C₆heteroalkyl. In some embodiments, the linker is optionally substituted with one or more substituents selected from halogen, —OH, oxo, —NH₂, —NHC₁-C₆alkyl, —N(C₁-C₆alkyl)₂, —C(═O)C₁-C₆alkyl, —C(═O)OH, —C(═O)NH₂, —C(═O)N(C₁-C₆alkyl)₂, —C(═O)NHC₁-C₆alkyl, and C₁-C₆alkyl. In some embodiments, the linker is optionally substituted with 1-5 substituents. In some embodiments, the linker is optionally substituted with 1-2 substituents.

In some embodiments, the linker of Formula (II-1) has a structure of Formula (II-1a),

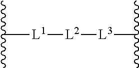

wherein
each of L¹ and L³ is independently —O—, —NR^{L}—, —N(R^{L})₂—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—; and
L² is absent, substituted or unsubstituted C₁-C₃₀ alkylene, or substituted or unsubstituted C₁-C₃₀ heteroalkylene.

In some embodiments, one of L¹, L², and L³ optionally comprises a residualizing agent or a non-residualizing agent. In some embodiments, L¹ is absent. In some embodiments, L³ is absent.

In some embodiments, the linker comprises a structure of Formula (II-1b),

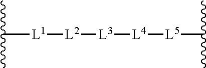

wherein each of L¹ and L⁵ is independently —O—, —NR^{L}—, —N(R^{L})₂—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted 5-6 membered cycloalkyl, or substituted or unsubstituted 5-6 membered heterocycloalkyl; and
L², L³ and L⁴ are each independently absent, substituted or unsubstituted 5-6 membered cycloalkyl, substituted or unsubstituted 5-6 membered heterocycloalky, substituted or unsubstituted C₁-C₃₀ alkylene, or substituted or unsubstituted C₁-C₃₀ heteroalkylene.

In some embodiments, L¹ is —NH—.

In some embodiments, L² is absent. In some embodiments, L² is substituted or unsubstituted C₁-C₃₀ alkylene, or substituted or unsubstituted C₁-C₃₀ heteroalkylene. In some embodiments, L² is substituted or unsubstituted C₁-C₃₀ alkylene. In some embodiments, L² is substituted or unsubstituted C₁-C₃₀ heteroalkylene. In some embodiments, L² is substituted or unsubstituted C₁-C₁₈ alkylene, or substituted or unsubstituted C₁-C₁₈ heteroalkylene. In some embodiments, L² is optionally substituted. In some embodiments, L² is optionally substituted with one or more substituents selected from —OH, —SH, oxo, amino, C₁-C₆ alkyl, C₁-C₆ hydroxyalkyl, C₁-C₆ haloalkyl, C₁-C₆ aminoalkyl, —C(═O)OR^{L}, —OC(═O) R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}. In some embodiments, L² is C₁-C₃₀ heteroalkylene that is optionally substituted with one or more substituents selected from —OH, —SH, oxo, amino, C₁-C₆ alkyl, C₁-C₆ hydroxyalkyl, C₁-C₆ haloalkyl, and C₁-C₆ aminoalkyl. In some embodiments, L² is optionally substituted with C₁-C₆ alkyl which is further optionally substituted with one or more substituents chosen from —OH, —SH, oxo, amino, C₆-C₁₀ aryl, 6- to 10-membered heteroaryl, —C(═O)OR^{L}, —OC(═O)R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}.

In some embodiments, L³ is —NH—. In some embodiments, L³ is absent.

In some embodiments, L⁴ is absent. In some embodiments, L⁴ is substituted or unsubstituted 5-6 membered cycloalkyl, substituted or unsubstituted 5-6 membered heterocycloalky, substituted or unsubstituted C₁-C₃₀ alkylene, or substituted or unsubstituted C₁-C₃₀ heteroalkylene.

In some embodiments, L⁵ is —NH—. In some embodiments, L⁵ is absent.

In some embodiments for Formula (II-1b), L¹ is —O—, —N(methyl)-, —NH— or —C(═O)—; L⁵ is —O—, —N(methyl)-, —NH— or —C(═O)—; L², L³ and L⁴ are each independently absent, substituted or unsubstituted 5-6 membered cycloalkyl, substituted or unsubstituted 5-6 membered heterocycloalky, substituted or unsubstituted C₁-C₁₂ alkylene, or substituted or unsubstituted C₁-C₃₀ heteroalkylene, wherein L¹ is connected to the metal chelator and L⁵ is connected to the EphA2 binding peptide. In some embodiments for Formula (II-1b), L¹ is —O—, —N(methyl)-, —NH— or —C(═O)—; L⁵ is —O—, —N(methyl)-, —NH— or —C(═O)—; L², L³ and L⁴ are each independently absent, substituted or unsubstituted 5-6 membered cycloalkyl, substituted or unsubstituted 5-6 membered heterocycloalky, substituted or unsubstituted C₁-C₁₂ alkylene, or substituted or unsubstituted C₁-C₃₀ heteroalkylene, wherein L¹ is connected to the radionuclide, residualizing agent, or non-residualizing agent, and L⁵ is connected to the EphA2 binding peptide.

In some embodiments for Formula (II-1b), L² is unsubstituted C₁-C₁₂ alkylene, and L³ and L⁴ are absent.

In some embodiments, the linker comprises substituted or unsubstituted C₁-C₃₀ alkylene, C₁-C₁₂ alkylene, C₁-C₈alkylene, C₁-C₆ alkylene, or C₂-C₆ alkylene. In some embodiments, the linker comprises C₂-C₆ alkylene. In some embodiments, the linker comprises C₄-C₆ alkylene.

In some embodiments, each of L¹ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L¹ is —O—, —NR^{L}—, —OP(═O) (OR)O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L¹ is —O—, —NH—, —S(═O)—, —S(═O)₂—, or —C(═O)—. In some embodiments, L¹ is —C(═O)NH— or —NHC(═O)—. In some embodiments, L¹ is substituted or unsubstituted C₃-C₁₅ cycloalkyl, or substituted or unsubstituted C₁-C₁₂ heterocycloalkyl. In some embodiments, L¹ is substituted or unsubstituted aryl or substituted or unsubstituted heteroaryl. In some embodiments, L¹ is substituted or unsubstituted C₁-C₃₀ alkylene. In some embodiments, L¹ is substituted or unsubstituted C₂-C₃₀ alkenylene. In some embodiments, L¹ is substituted or unsubstituted C₁-C₃₀ heteroalkylene. In some embodiments, L¹ is substituted or unsubstituted C₅-C₂₅ heteroalkylene. In some embodiments, L¹ is substituted or unsubstituted C₅-C₁₂ heteroalkylene.

In some embodiments, each of L² is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L² is —O—, —NR^{L}—, —OP(═O) (OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L² is —O—, —NH—, —S(═O)—, —S(═O)₂—, or —C(═O)—. In some embodiments, L² is —C(═O)NH— or —NHC(═O)—. In some embodiments, L² is substituted or unsubstituted C₃-C₁₅ cycloalkyl, or substituted or unsubstituted C₁-C₁₂ heterocycloalkyl. In some embodiments, L² is substituted or unsubstituted aryl or substituted or unsubstituted heteroaryl. In some embodiments, L² is substituted or unsubstituted C₁-C₃₀ alkylene. In some embodiments, L² is substituted or unsubstituted C₂-C₃₀ alkenylene. In some embodiments, L² is substituted or unsubstituted C₁-C₃₀ heteroalkylene. In some embodiments, L² is substituted or unsubstituted C₅-C₂₅ heteroalkylene. In some embodiments, L² is substituted or unsubstituted C₅-C₁₂ heteroalkylene.

In some embodiments, each of L³ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L³ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L³ is —O—, —NH—, —S(═O)—, —S(═O)₂—, or —C(═O)—. In some embodiments, L³ is —C(═O)NH— or —NHC(═O)—. In some embodiments, L³ is substituted or unsubstituted C₃-C₁₅ cycloalkyl, or substituted or unsubstituted C₁-C₁₂ heterocycloalkyl. In some embodiments, L³ is substituted or unsubstituted aryl or substituted or unsubstituted heteroaryl. In some embodiments, L³ is substituted or unsubstituted C₁-C₃₀ alkylene. In some embodiments, L³ is substituted or unsubstituted C₂-C₃₀ alkenylene. In some embodiments, L³ is substituted or unsubstituted C₁-C₃₀ heteroalkylene. In some embodiments, L³ is substituted or unsubstituted C₅-C₂₅ heteroalkylene. In some embodiments, L³ is substituted or unsubstituted C₅-C₁₂ heteroalkylene. In some embodiments, L³ is absent.

In some embodiments, each of L⁴ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L⁴ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L⁴ is —O—, —NH—, —S(═O)—, —S(═O)₂—, or —C(═O)—. In some embodiments, L⁴ is —C(═O)NH— or —NHC(═O)—. In some embodiments, L⁴ is substituted or unsubstituted C₃-C₁₅ cycloalkyl, or substituted or unsubstituted C₁-C₁₂ heterocycloalkyl. In some embodiments, L⁴ is substituted or unsubstituted aryl or substituted or unsubstituted heteroaryl. In some embodiments, L⁴ is substituted or unsubstituted C₁-C₃₀ alkylene. In some embodiments, L⁴ is substituted or unsubstituted C₂-C₃₀ alkenylene. In some embodiments, L⁴ is substituted or unsubstituted C₁-C₃₀ heteroalkylene. In some embodiments, L⁴ is substituted or unsubstituted C₅-C₂₅ heteroalkylene. In some embodiments, L⁴ is substituted or unsubstituted C₅-C₁₂ heteroalkylene. In some embodiments, L⁴ is absent.

In some embodiments, each of L⁵ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —CR^{L}—N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L⁵ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L⁵ is —O—, —NH—, —S(═O)—, —S(═O)₂—, or —C(═O)—. In some embodiments, L⁵ is —C(═O)NH— or —NHC(═O)—. In some embodiments, L⁵ is substituted or unsubstituted C₃-C₁₅ cycloalkyl, or substituted or unsubstituted C₁-C₁₂ heterocycloalkyl. In some embodiments, L⁵ is substituted or unsubstituted aryl or substituted or unsubstituted heteroaryl. In some embodiments, L⁵ is substituted or unsubstituted C₁-C₃₀ alkylene. In some embodiments, L⁵ is substituted or unsubstituted C₂-C₃₀ alkenylene. In some embodiments, L⁵ is substituted or unsubstituted C₁-C₃₀ heteroalkylene. In some embodiments, L⁵ is substituted or unsubstituted C₅-C₂₅ heteroalkylene. In some embodiments, L⁵ is substituted or unsubstituted C₅-C₁₂ heteroalkylene. In some embodiments, L⁵ is absent.

In some embodiments, each of L¹, L², L³, L⁴ or L⁵ is optionally substituted with one or more substituents selected from halogen, —CN, oxo, —OH, —OC₁-C₆alkyl, SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NR^{b}C(═NR^{b})NR^{c}R^{d}, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, and C₁-C₆heteroalkyl. In some embodiments, each of L¹, L², L³, L⁴ or L⁵ is optionally substituted with one or more substituents selected from halogen, —CN, —OH, oxo, —OC₁-C₆alkyl, —NH₂, —NHC₁-C₆alkyl, —N(C₁-C₆alkyl)₂, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, and C₁-C₆heteroalkyl.

In some embodiments, the linker has a structure of

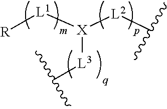

wherein each L¹, L², and L³ is independently —O—, —NR^{L}—, —N(R^{L})₂⁺—, —OP(═O) (OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, substituted or unsubstituted C₁-C₁₅ arylene, —(C₁-C₃₀ alkylene)-O—, —O—(C₁-C₃₀ alkylene)-,
(C₁-C₃₀ alkylene)-NR^{L}—, —NR^{L}-(C₁-C₃₀ alkylene)-, —(C₁-C₃₀ alkylene)-N(R^{L})₂⁺—, —N(R^{L})₂⁺—(C₁-C₃₀ alkylene)-, or a click chemistry residue; and
R is hydrogen, azide, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted cycloalkynyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;
R^{L} is hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;
X is N or CR^{L}; and
   each of m, p, and q is independently 0, 1, 2, 3, 4, 5, 6, 7, 8, 9, 10, 11, 12, 13, 14, or 15.

In some embodiments, the linker has a structure of

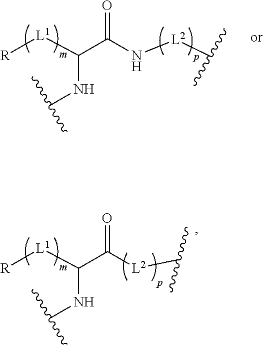

wherein
wherein each L¹ and L² is independently —O—, —NR^{L}—, —N(R^{L})₂⁺—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₂₀ alkylene, or —(CHR^{L}—CHR^{L}—O)₁₋₁₀—;
R^{L} is hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, or substituted or unsubstituted C₂-C₇ heterocycloalkyl;
R is hydrogen, azide, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted cycloalkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl;
m is 1 to 10; and p is 0-3.

In some embodiments, L² is —O—, —NR^{L}—, —N(R^{L})₂⁺—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, or —(CH₂—CH₂—O)₁₋₆—;
L¹ is —O—, —NR^{L}—, —N(R^{L})₂⁺—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₂₀ alkylene, or —(CH₂—CH₂—O)₁₋₆—;
R^{L} is hydrogen or substituted or unsubstituted C₁-C₄ alkyl;
R is hydrogen, substituted or unsubstituted C₁-C₄ alkyl, substituted or unsubstituted C₃-C₃₀ cycloalkyl, substituted or unsubstituted C₂-C₃₀ heterocycloalkyl, substituted or unsubstituted aryl, or substituted or unsubstituted heteroaryl;
m is 1 to 10; and p is 0-3.

In some embodiments, the linker has a structure of

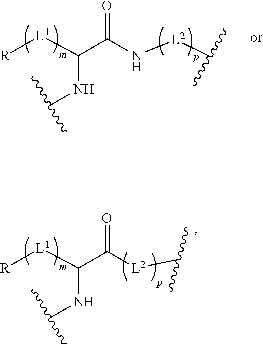

wherein
L² is —O—, —NR^{L}—, —N(R^{L})₂⁺—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, or —(CH₂—CH₂—O)₁₋₆—;
L¹ is —O—, —NR^{L}—, —N(R^{L})₂⁺—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₂₀ alkylene, or —(CH₂—CH₂—O)₁₋₆—;
R^{L} is hydrogen or substituted or unsubstituted C₁-C₄ alkyl;
R is hydrogen, substituted or unsubstituted C₁-C₄ alkyl, substituted or unsubstituted C₃-C₃₀ cycloalkyl, substituted or unsubstituted C₂-C₃₀ heterocycloalkyl, substituted or unsubstituted C₆-C₁₀ aryl, or substituted or unsubstituted C5-C₉ heteroaryl;
m is 1 to 4; and p is 0-3.

In some embodiments, R is hydrogen, substituted or unsubstituted C₆-C₁₀ aryl, substituted or unsubstituted C5-C9 heteroaryl, or a sterol.

In some embodiments, at least one L¹ is unsubstituted C₃-C₂₀ alkylene.

In some embodiments, the linker comprises one or more of a substituted or unsubstituted C₆-C₁₀ aryl, substituted or unsubstituted C5-C9 heteroaryl, a sterol, sulfonamide, phosphate ester, polyethylene glycol, or C₃-C₂₀ alkylene, or amino acid residues.

In some embodiments, the linker comprises one or more selected from AEEA, AEEP, AEEEP, and AEEEEP groups. In some embodiments, the linker comprises

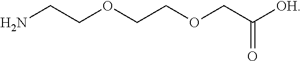 [SMILES: NCCOCCOCC(=O)O]

In some embodiments, the linker comprises

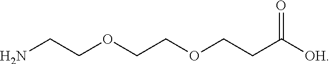 [SMILES: NCCOCCOCCC(=O)O]

In some embodiments, the linker comprises

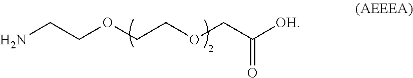

In some embodiments, the linker comprises

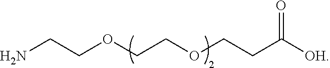

In some embodiments, the linker comprises

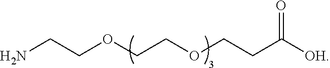

In some embodiments, the linker is

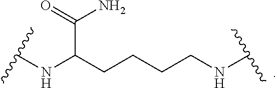 [SMILES: *NCCCCC(N*)C(N)=O]

In some embodiments, the linker is or comprises lysine. In some embodiments, the linker comprises C₁-C₁₂ alkylene. In some embodiments, the linker comprises C3-C₉ alkylene. In some embodiments, the linker comprises C₂-C₈ alkylene. In some embodiments, the linker comprises 1 to 10 repeating ethylene glycol units. In some embodiments, the linker comprises 2 to 4 repeating ethylene glycol units. In some embodiments, the linker comprises 5 to 8 repeating ethylene glycol units. In some embodiments, the linker comprises NH₂—(CH₂) n-COOH, wherein n is 1 to 12. In some embodiments, the linker comprises NH₂—(CH₂)₂—COOH). In some embodiments, the linker comprises NH₂—(CH₂)₃—COOH). In some embodiments, the linker comprises NH₂—(CH₂)₄—COOH). In some embodiments, the linker comprises NH₂—(CH₂)₅—COOH). In some embodiments, the linker comprises NH₂—(CH₂)₆—COOH). In some embodiments, the linker comprises NH₂—(CH₂)₇—COOH). In some embodiments, the linker comprises NH₂—(CH₂)₈—COOH). In some embodiments, the linker comprises NH₂—(CH₂)₁₀—COOH). In some embodiments, the linker is absent.

In some embodiments, a linker of the present disclosure (e.g., a linker of Formula (II-1), (II-1a) or (II-1b)) comprises a structure of

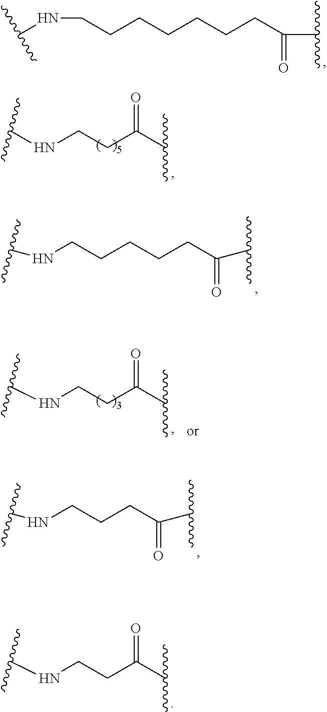

In some embodiments, a linker of the present disclosure comprises a structure of

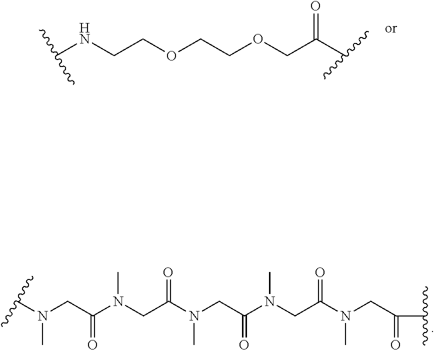 [SMILES: *NCCOCCOCC(*)=O ; *C(=O)CN(C)C(=O)CN(C)C(=O)CN(C)C(=O)CN(C)C(=O)CN(*)C]

In some embodiments, a linker of the present disclosure (e.g., a linker of Formula (II-1), (II-1a) or (II-1b)) comprises a structure of

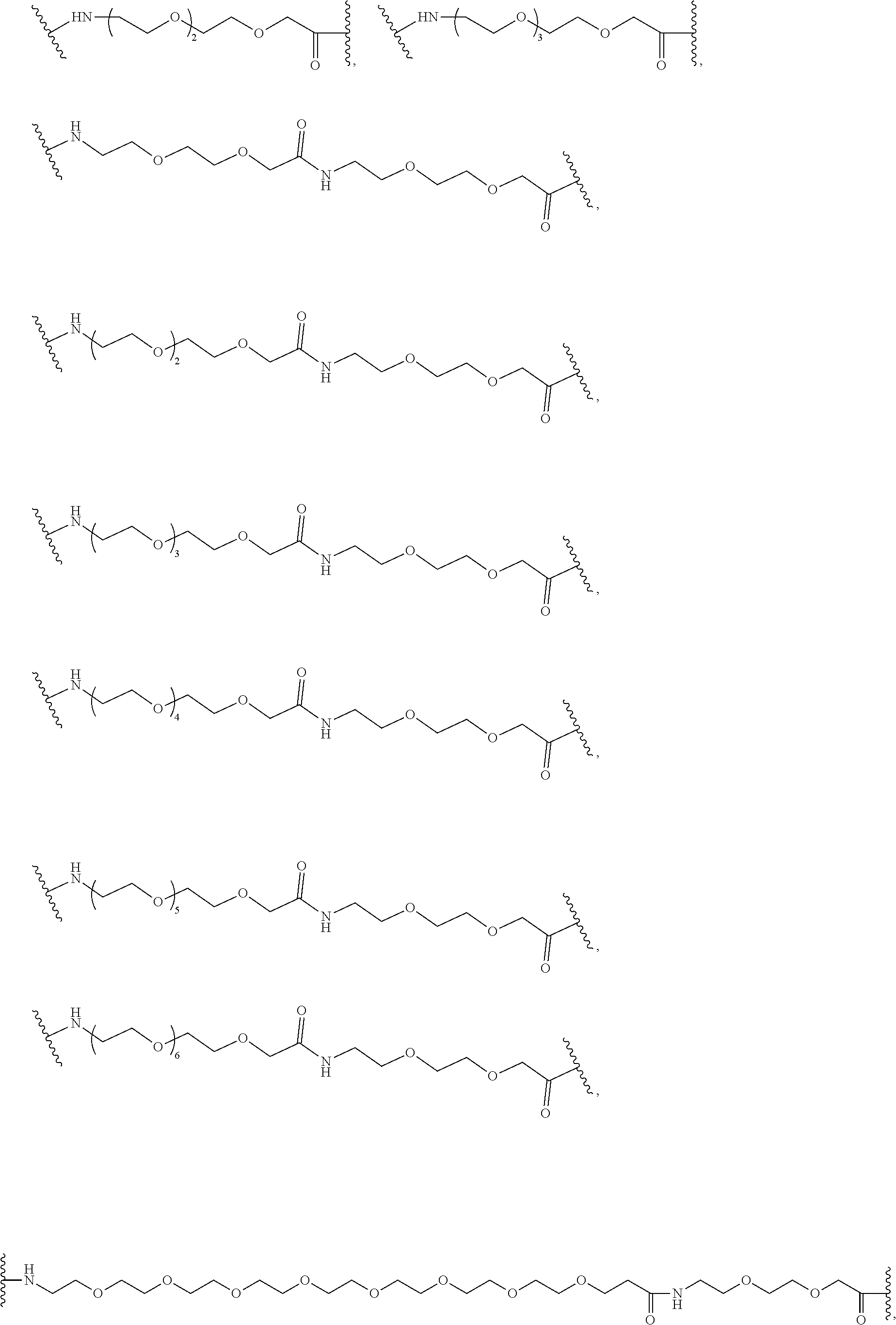

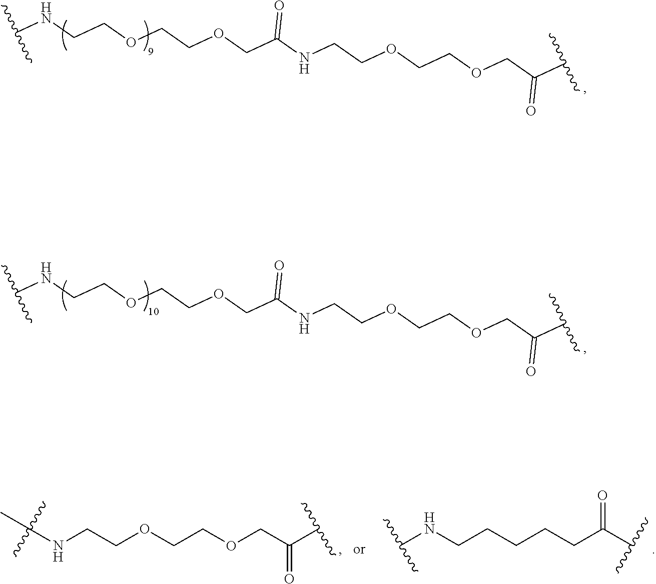

In some embodiments, the linker comprises a structure selected from:

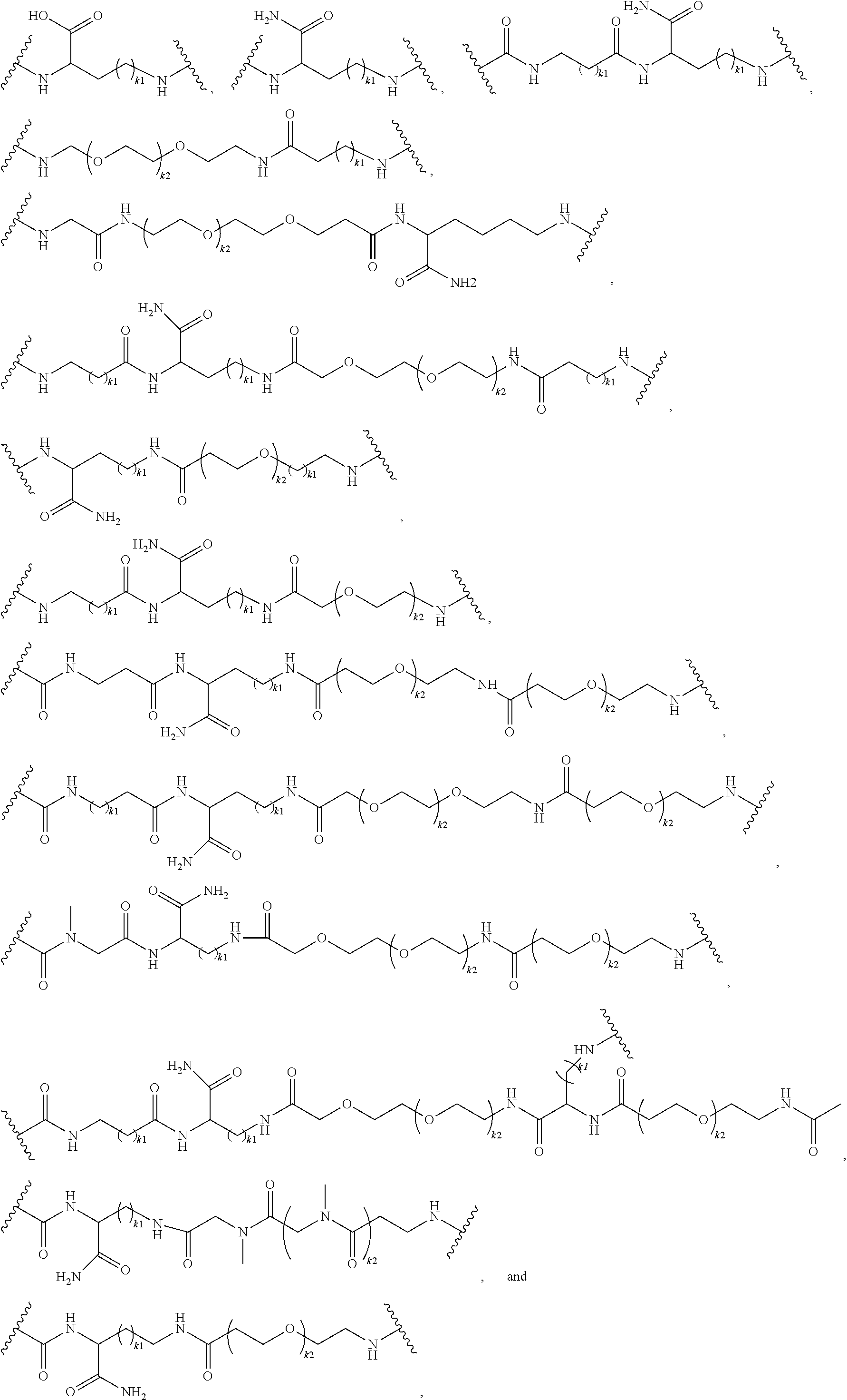

wherein each k1 is independently 0 or an integer from 1 to 20; and each k2 is independently 0 or an integer from 1 to 15. In some embodiments, k1 is 0. In some embodiments, k1 is 1. In some embodiments, k1 is 2. In some embodiments, k1 is 3. In some embodiments, k1 is 4. In some embodiments, k1 is 5. In some embodiments, k1 is 6. In some embodiments, k1 is 7. In some embodiments, k1 is 8. In some embodiments, k1 is 9. In some embodiments, k1 is 10. In some embodiments, k1 is 11. In some embodiments, k1 is 12. In some embodiments, k1 is 13. In some embodiments, k1 is 14. In some embodiments, k1 is 15. In some embodiments, k1 is 16. In some embodiments, k1 is 17. In some embodiments, k1 is 18. In some embodiments, k1 is 19. In some embodiments, k1 is 20. In some embodiments, k2 is 0. In some embodiments, k2 is 1. In some embodiments, k2 is 2. In some embodiments, k2 is 3. In some embodiments, k2 is 4. In some embodiments, k2 is 5. In some embodiments, k2 is 6. In some embodiments, k2 is 7. In some embodiments, k2 is 8. In some embodiments, k2 is 9. In some embodiments, k2 is 10. In some embodiments, k2 is 11. In some embodiments, k2 is 12. In some embodiments, k2 is 13. In some embodiments, k2 is 14. In some embodiments, k2 is 15.

In some embodiments, the linker comprises a structure selected from:

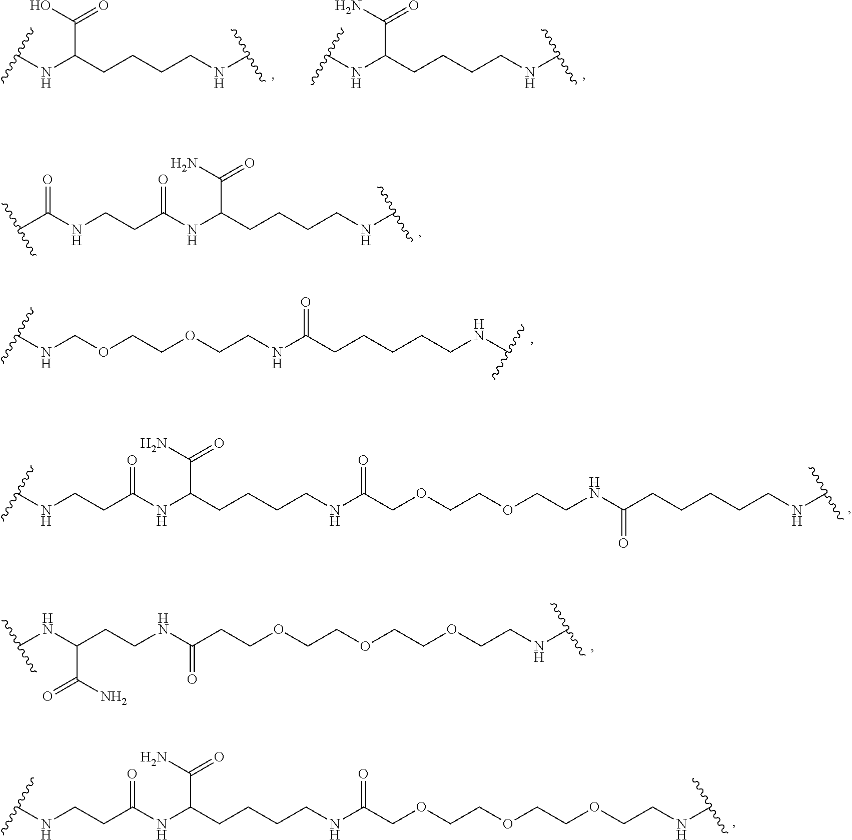

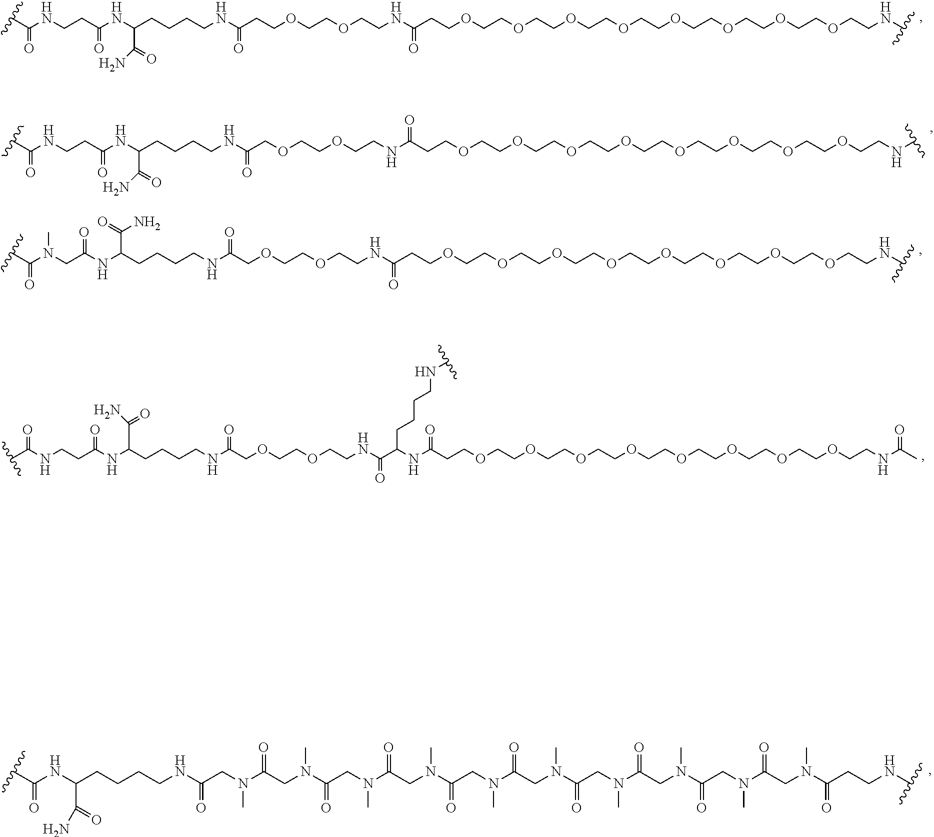 [SMILES: *NCCOCCOCCOCCOCCOCCOCCOCCOCCC(=O)NCCOCCOCCC(=O)NCCCCC(NC(=O)CCNC(*)=O)C(N)=O ; *NCCOCCOCCOCCOCCOCCOCCOCCOCCC(=O)NCCOCCOCC(=O)NCCCCC(NC(=O)CCNC(*)=O)C(N)=O ; *NCCOCCOCCOCCOCCOCCOCCOCCOCCC(=O)NCCOCCOCC(=O)NCCCCC(NC(=O)CN(C)C(*)=O)C(N)=O ; *NCCCCC(NC(=O)CCOCCOCCOCCOCCOCCOCCOCCOCCNC(C)=O)C(=O)NCCOCCOCC(=O)NCCCCC(NC(=O)CCNC(*)=O)C(N)=O ; *NCCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)NCCCCC(NC(*)=O)C(N)=O]

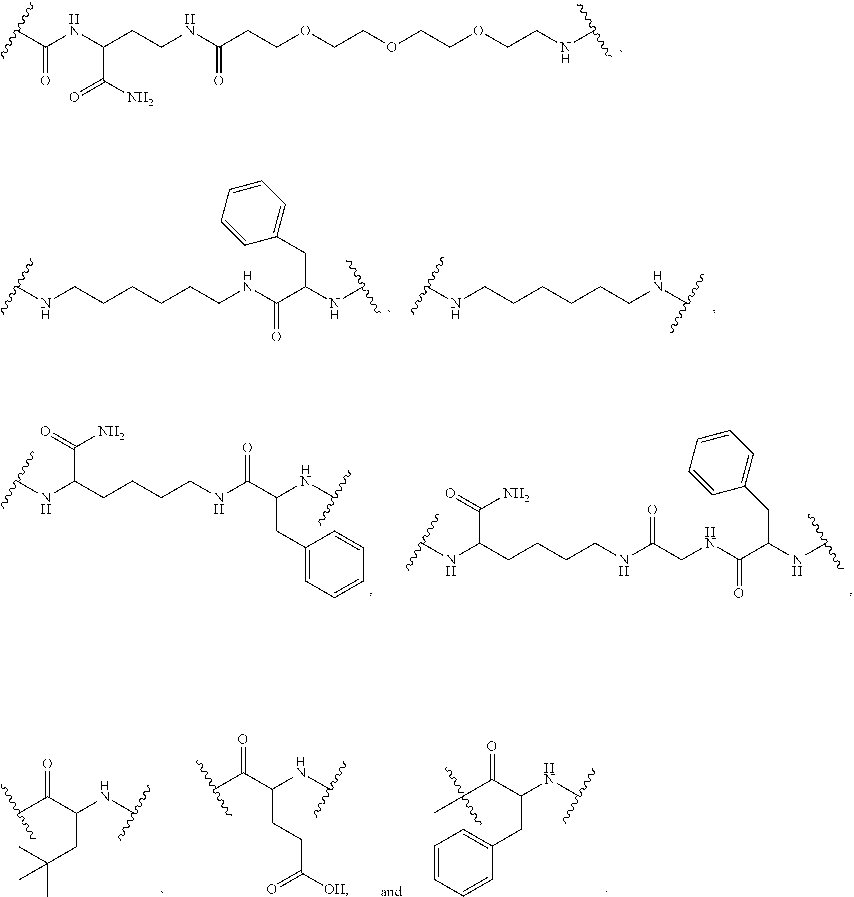

In some embodiments, the linker comprises a structure selected from:

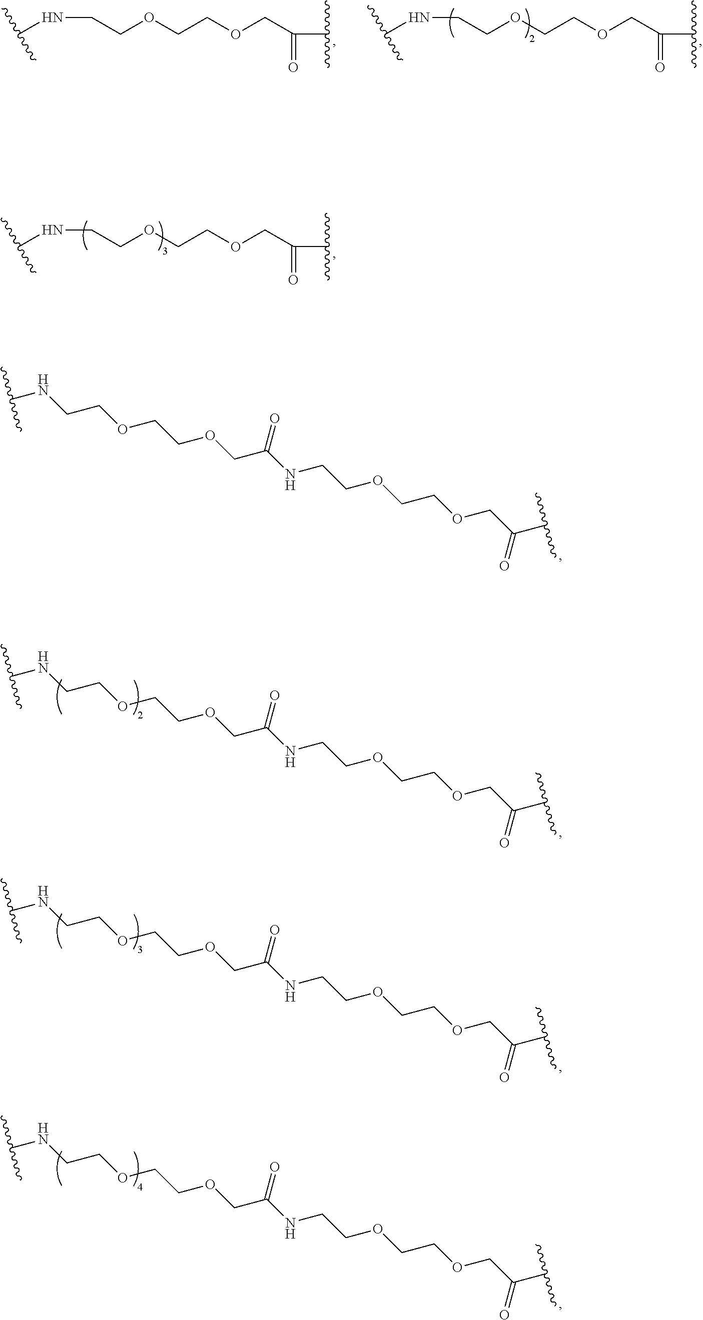

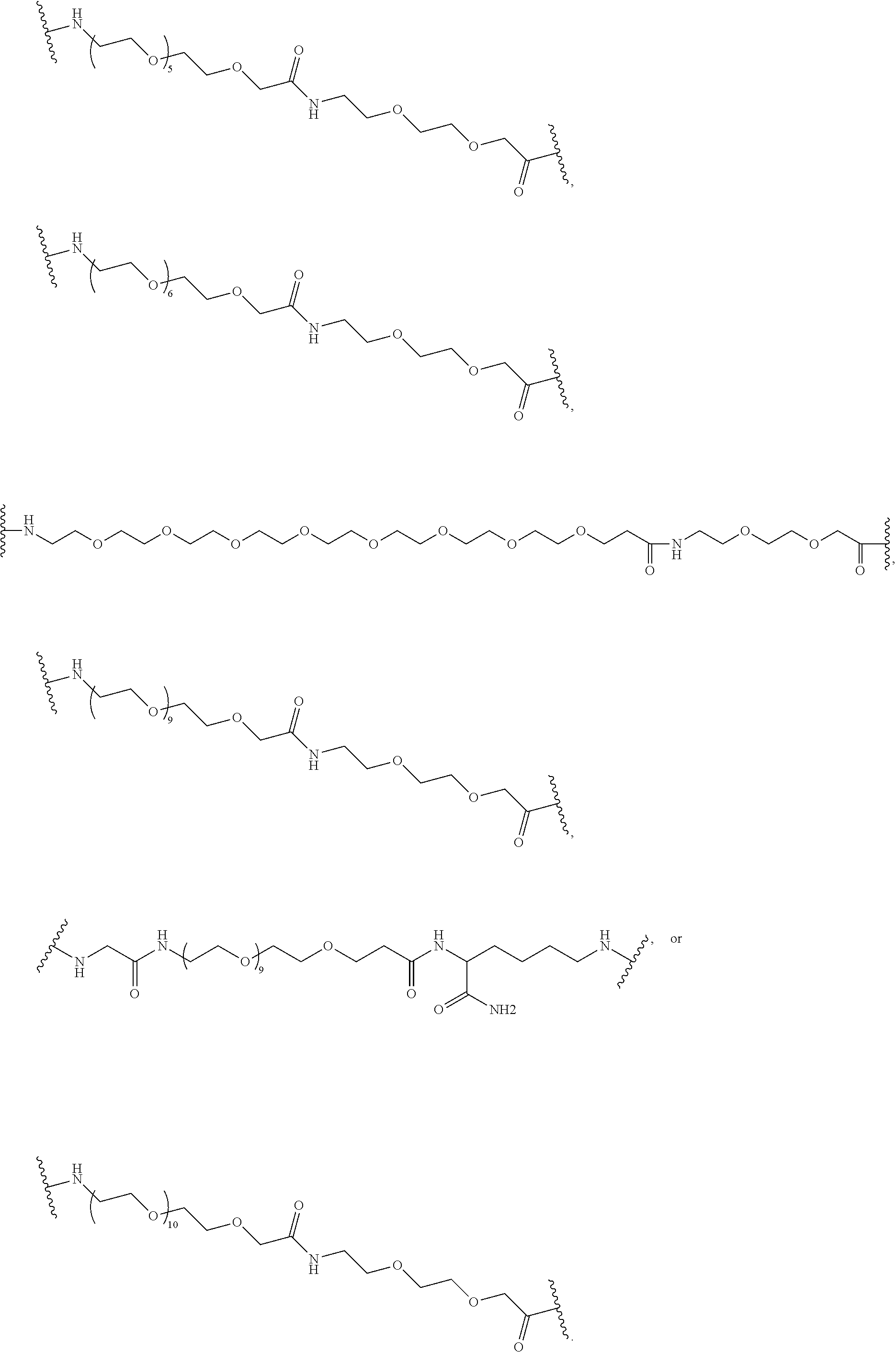

In some embodiments, the linker is configured to reversibly bind to a plasma protein such as albumin. In some embodiments, a dissociation constant (Kd) between the linker and human serum albumin is at most 15 μM, as determined at room temperature in human serum condition. In some embodiments, the Kd is from about 0.1 nM to about 10 μM. In some embodiments, the Kd is from about 10 nM to about 10 μM. In some embodiments, the Kd is from about 50 nM to about 1 μM. In some embodiments, the Kd is from about 100 nM to about 10 μM.

In some embodiments, a conjugate comprises a linker structure selected from Table 6.

TABLE 6

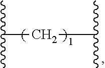

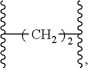

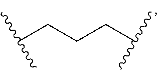

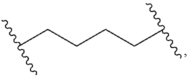

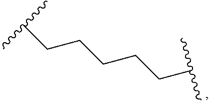

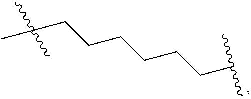

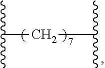

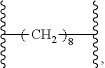

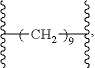

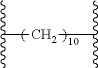

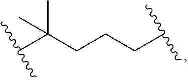

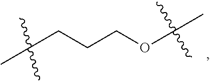

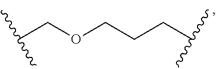

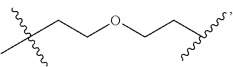

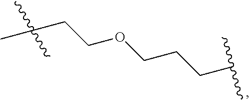

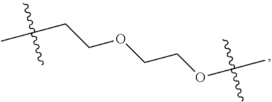

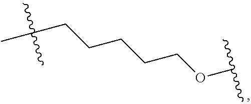

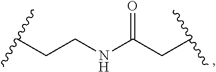

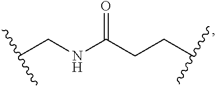

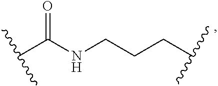

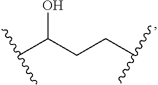

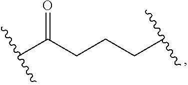

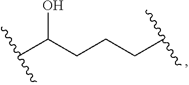

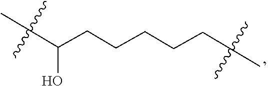

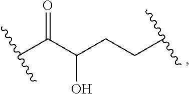

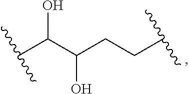

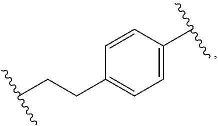

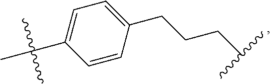

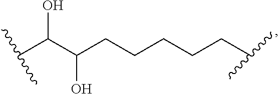

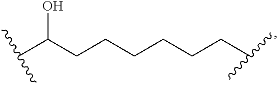

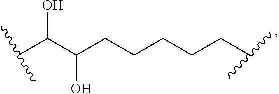

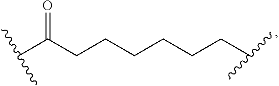

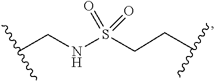

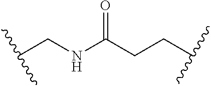

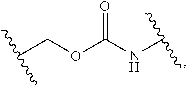

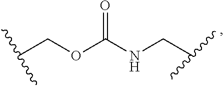

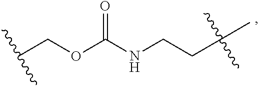

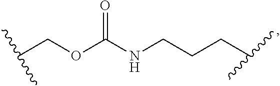

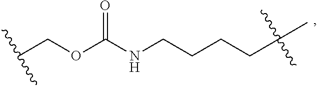

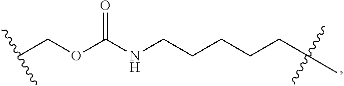

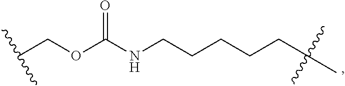

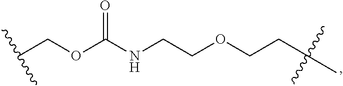

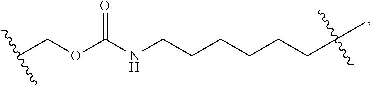

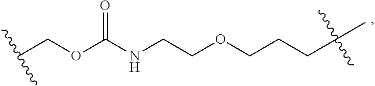

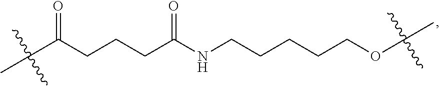

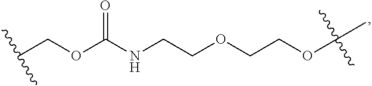

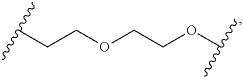

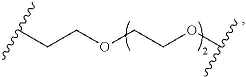

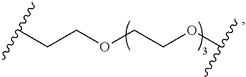

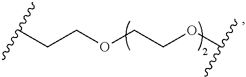

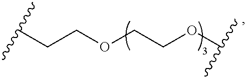

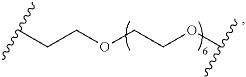

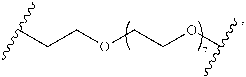

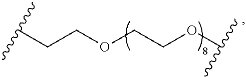

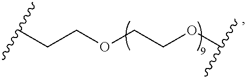

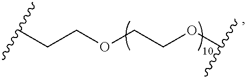

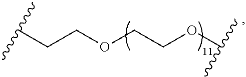

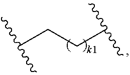

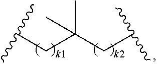

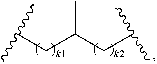

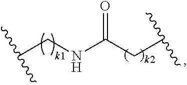

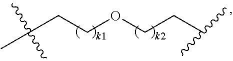

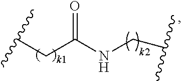

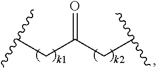

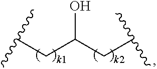

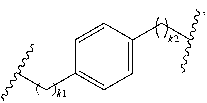

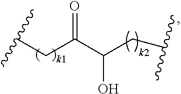

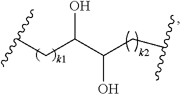

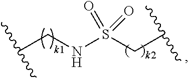

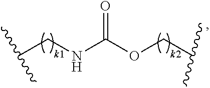

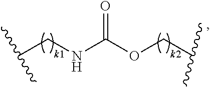

wherein each k1 and k2 is independently 0 or an integer selected from 1 to 20.

In some embodiments of Table 6, k1 is selected from 0-12. In some embodiments, k1 is 0. In some embodiments, k1 is 1. In some embodiments, k1 is 2. In some embodiments, k1 is 3. In some embodiments, k1 is 4. In some embodiments, k1 is 5. In some embodiments, k1 is 6. In some embodiments, k1 is 7. In some embodiments, k1 is 8. In some embodiments, k1 is 9. In some embodiments, k1 is 10. In some embodiments of Table 6, k2 is selected from 0-12. In some embodiments, k2 is 0. In some embodiments, k2 is 1. In some embodiments, k2 is 2. In some embodiments, k2 is 3. In some embodiments, k2 is 4. In some embodiments, k2 is 5. In some embodiments, k2 is 6. In some embodiments, k2 is 7. In some embodiments, k2 is 8. In some embodiments, k2 is 9. In some embodiments, k2 is 10.

In some embodiments, a linker of the present disclosure comprises

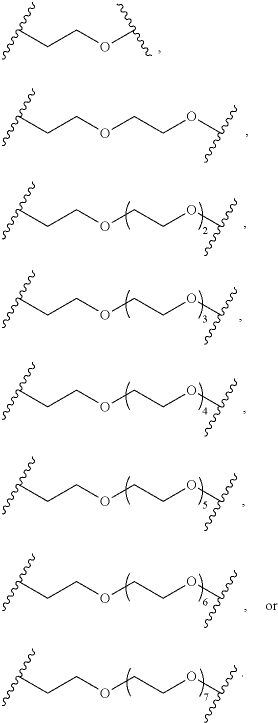

In some embodiments, a linker of the present disclosure comprises

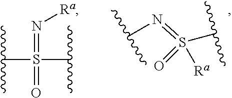

In some embodiments, a linker of the present disclosure comprises 1 to 20 groups independently selected from —CR^{b}R^{b}—, —C(═O)—, —S(═O)—, —S(═O)₂—, —NR^{a}—,

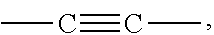

—CR^{b}═CR^{b}—,

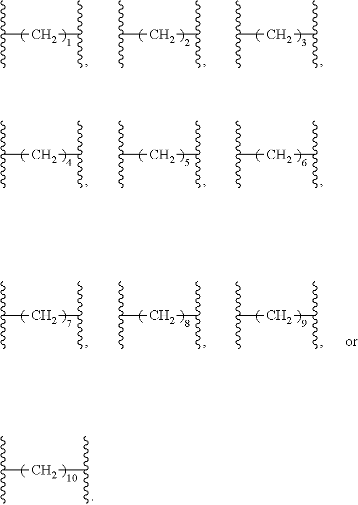

—O—, —S—, —C(═O)O—, —OC(═O)—, —C(═O)NR^{a}—, —NR^{a}C(═O)—, —S(═O)₂NR^{a}—, —NR^{a}S(═O)₂—, —NR^{a}C(═O)NR^{a}—, —NR^{a}C(═O)O—, —OC(═O)NR^{a}—, arylene, heteroarylene,
wherein each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl, and
wherein each R^{b} is independently hydrogen, halogen, —CN, —NO₂, —OR^{a}, —SR^{a}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl. In some embodiments, a linker of the present disclosure comprises 1 to 5, 1 to 3, or 1 to 10 groups as described above.

In some embodiments, the linker is a bond.

### Metal Chelator

In one aspect, described herein are conjugates that comprise a metal chelator that is configured to bind with a radionuclide. The metal chelator can refer to a moiety of the conjugate that is configured to bind with a radionuclide. In some embodiments, a conjugate described herein comprises two or more independent metal chelators, e.g., 2, 3, 4, 5, or more metal chelators. In some embodiments, a conjugate described herein comprises two metal chelators, which can be the same or different. In some embodiments, a conjugate described herein comprises two or more metal chelators. In some embodiments, the conjugate comprises two radionuclides bound to the metal chelators. The metal chelator can be attached to the linker or the peptide through any suitable group/atom of the chelator.

In some embodiments, the metal chelator is capable of binding a radioactive atom. The binding can be direct, e.g., the metal chelator can make hydrogen bonds or electrostatic interactions with the radioactive atom. The binding can also be indirect, e.g., the metal chelator binds to a molecule that comprises a radioactive atom. In some embodiments, the metal chelator comprises, or is, a macrocycle. In some embodiments, the metal chelator comprises, or is, 2,2′,2″,2″-(1,4,7,10-Tetraazacyclododecane-1,4,7,10-tetrayl)tetraacetic acid (DOTA) or 1,4,7-triazacyclononane-1,4,7-triacetic acid (NOTA). In some embodiments, the metal chelator comprises a macrocycle, e.g., a macrocycle comprising an O and/or a N, DOTA, NOTA, one or more amines, one or more ethers, one or more carboxylic acids, EDTA, DTPA, TETA, DO3A, PCTA, or desferrioxamine.

In some embodiments, the metal chelator comprises a plurality of amines. In some embodiments, the metal chelator includes 4 or more N, 4 or more carboxylic acid groups, or a combination thereof. In some embodiments, the metal chelator does not comprise S. In some embodiments, the metal chelator comprises a ring. In some embodiments, the ring comprises an O and/or an N. In some embodiments, the metal chelator is a ring that includes 3 or more N, 3 or more carboxylic acid groups, or a combination thereof. In some embodiments, the metal chelator is polydentate.

In some embodiments, a metal chelator described herein is selected from: DOTA, DOTA-GA, pBn-DOTA, pBn-SCN-DOTA, NH2-DOTA, NH2-DOTA-GA, p-NCS-Bn-DOTA-GA, p-NH2-Bn-oxo-DO3A, p-SCN-Bn-oxo-DO3A, NOTA, NODA-GA, NH2-NODA-GA, p-NCS-Bn-NODA-GA, p-NH2-Bn-NOTA, p-SCN-Bn-NOTA, NCS-MP-NODA, NH2-MPAA-NODA, PCTA, p-NH2-Bn-PCTA, p-SCN-Bn-PCTA, p-SCN-Bn-HEHA, H2-MACROPA-NCS, H1-MACROPA, H2-MACROPA-NH2, H4-OCTAPA, tetra-(S, S, S, S)-Me-DOTA, tetra-(S, S, S, S)-Et-DOTA, tetra-(S, S, S, S)-iBu-DOTA, or maleimide-nBu-DOTA.

In some embodiments, a metal chelator described herein has a structure of

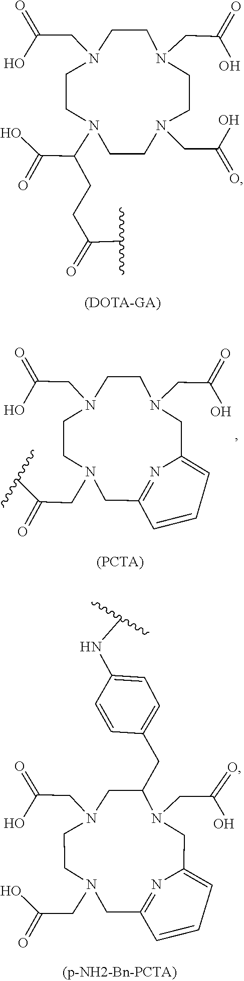 [SMILES: *C(=O)CCC(C(=O)O)N1CCN(CC(=O)O)CCN(CC(=O)O)CCN(CC(=O)O)CC1 ; *C(=O)CN1CCN(CC(=O)O)CCN(CC(=O)O)Cc2cccc(n2)C1 ; *Nc1ccc(CC2CN(CC(=O)O)CCN(CC(=O)O)Cc3cccc(n3)CN2CC(=O)O)cc1]

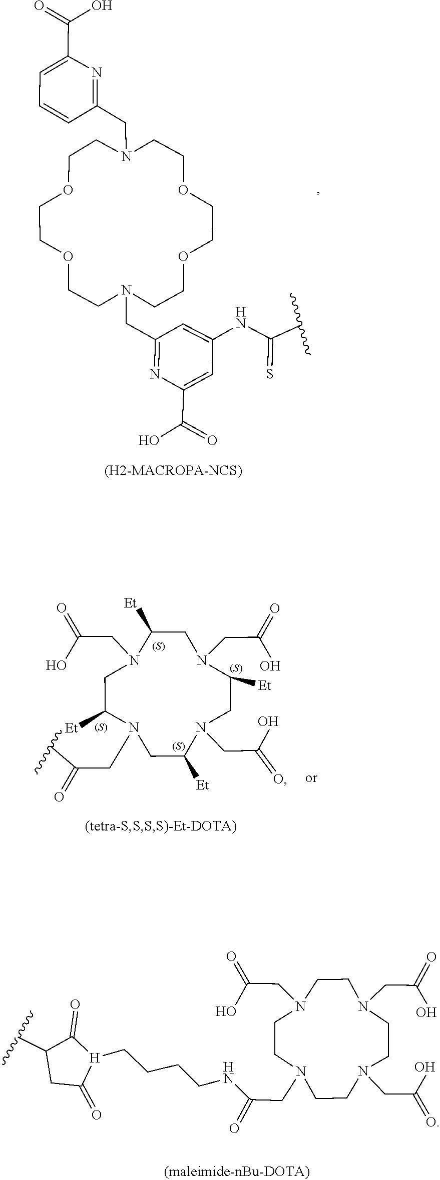

In some embodiments, a metal chelator described herein has a structure of

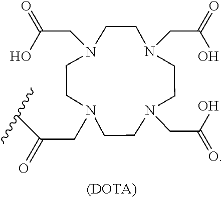

In some embodiments, a metal chelator described herein comprises a cyclic chelating agent. Exemplary cyclic chelating agents include, but are not limited to, AAZTA, BAT, BAT-TM, Crown, Cyclen, DO2A, CB-DO2A, DO3A, H3HP-DO3A, Oxo-DO3A, p-NH₂-Bn-Oxo-DO3A, DOTA, DOTA-3py, DOTA-PA, DOTA-GA, DOTA-4AMP, DOTA-2py, DOTA-1py, p-SCN-Bn-DOTA, CHX-A″-EDTA, MeO-DOTA-NCS EDTA, DOTAMAP, DOTAGA, DOTAGA-anhydride, DOTMA, DOTASA, DOTAM, DOTP, CB-Cyclam, TE2A, CB-TE2A, CB-TE2P, DM-TE2A, MM-TE2A, NOTA, NOTP, HEHA, HEHA-NCS, p-SCN-Bn-HEHA, DTPA, CHX-A″-DTPA, p-NH₂-Bn-CHX-A″-DTPA, p-SCN-DTPA, p-SCN-Bz-Mx-DTPA, 1B4M-DTPA, p-SCN-Bn1B-DTPA, p-SCN-Bn-1B4M-DTPA, p-SCN-Bn-CHX-A″-DTPA, PEPA, p-SCN-Bn-PEPA, TETPA, DOTPA, DOTMP, DOTPM, t-Bu-calix[4]arene-tetracarboxylic acid, macropa, macropa-NCS, macropid, H₃L¹, H₃L⁴, H₂azapa, H₅decapa, bispa², H₄pypa, H₄octapa, H₄CHXoctapa, p-SCN-Bn-H₄octapa, p-SCN-Bn-H₄octapa, TTHA, p-NO₂-Bn-neunpa, H₄octox, H₂macropa, H₂bispa², H₄phospa, H₆phospa, p-SCN-Bn-H₆phospa, TETA, p-NO₂-Bn-TETA, TRAP, TPA, HBED, SHBED, HBED-CC, (HBED-CC) TFP, DMSA, DMPS, DHLA, lipoic acid, TGA, BAL, Bis-thioseminarabazones, p-SCN-NOTA, nNOTA, NODAGA, CB-TE1A1P, 3P-C-NETA-NCS, 3p-C-DEPA, 3P-C-DEPA-NCS, TCMC, PCTA, NODIA-Me, TACN, pycup1A1B, pycup2A, THP, DEDPA, H₂DEDPA, p-SCN-Bn-H₂DEDPA, p-SCN-Bn-TCMC, motexafin, NTA, NOC, 3p-C-NETA, p-NH₂-Bn-TE3A, SarAr, DiAmSar, SarAr-NCS, AmBaSar, BaBaSar, TACN-TM, CP256, C-NE3TA, C-NE3TA-NCS, NODASA, NETA-monoamide, C-NETA, NOPO, BPCA, p-SCN-Bn-DFO, DFO-ChX-Mal, DFO, DFO-IAC, DFO-BAC, DiP-LICAM, EC, SBAD, BAPEN, TACHPYR, NEC-SP, LPy, L¹, L², L³, and EuK-106. In some embodiments, the metal chelator is DOTA, TRITA, TETA, DOTA-MA, DO3A-HP, DOTMA, DOTA-pNB, DOTP, DOTMP, DOTEP, DOTMPE, F-DOTPME, DOTPP, DOTBzP, DOTA-monoamide, p-NCS-DOTA, p-NCS-PADOTA, BAT, DO3TMP-Monoamide, p-NCS-TRITA, NOTA, or CHX-A″-DTPA. In some embodiments, a metal chelator described herein comprises an acyclic chelating agent. Exemplary acyclic chelating agents include, but are not limited to, DTA, CyEDTA, EDTMP, DTPMP, DTPA, CyDTPA, Cy2DTPA, DTPA-MA, DTPA-BA, and BOPA. In some embodiments, a metal chelator described herein comprises DOTA, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP. In some embodiments, a metal chelator described herein comprises H₄pypa, H₄octox, H₄octapa, p-NO₂-Bn-neunpa, p-SCN-Bn-H₄neunpa, TTHA, ^{t}Bu₄pypa-C7-NHS, H₄neunpa, H₂macropa, HP-DO3A, BT-DO3A, DO3A-Nprop, DO3AP, DO2A2P, DOA3P, DOTP, DOTPMB, DOTAMAE, DOTAMAP, DO3AM^{Bu}, DOTMA, TCE-DOTA, DEPA, PCTA, p-NO₂-Bn-PCTA, p-NO₂-Bn-DOTA, symPC2APA, symPCA2PA, asymPC2APA, asymPCA2PA, TRAP, AAZTA, DATA^{m}, THP, HEHA, or HBED.

In some embodiments, the metal chelator is DO3A. In some embodiments, the metal chelator is PEPA. In some embodiments, the metal chelator is EDTA. In some embodiments, the metal chelator is CHX-A″-DTPA. In some embodiments, the metal chelator is HEHA. In some embodiments, the metal chelator is DOTMP. In some embodiments, the metal chelator is t-Bu-calix[4]arene-tetracarboxylic acid. In some embodiments, the metal chelator is macropa. In some embodiments, the metal chelator is macropa-NCS. In some embodiments, the metal chelator is H₄pypa. In some embodiments, the metal chelator is H₄octapa. In some embodiments, the metal chelator is H₄CHXoctapa. In some embodiments, the metal chelator is DOTP. In some embodiments, the metal chelator is crown.

In some embodiments, the metal chelator is DOTA. In some embodiments, the metal chelator is a chiral derivative of DOTA. Exemplary chiral DOTA chelators are described in Dai et al., Nature Communications (2018) 9:857. In some embodiments, the metal chelator is 2,2′,2″,2′″-((2S,5S,8S,11S)-2,5,8,11-tetramethyl-1,4,7,10-tetraazacyclododecane-1,4,7,10-tetrayl)tetraacetic acid. In some embodiments, the metal chelator has a structure of

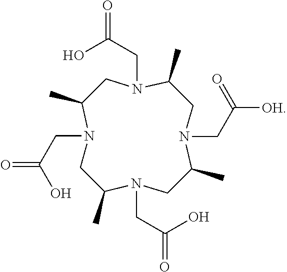 [SMILES: C[C@H]1CN(CC(=O)O)[C@@H](C)CN(CC(=O)O)[C@@H](C)CN(CC(=O)O)[C@@H](C)CN1CC(=O)O]

In some embodiments, the metal chelator is 2,2′,2″,2′″-((2S,5S,8S,11S)-2,5,8,11-tetraethyl-1,4,7,10-tetraazacyclododecane-1,4,7,10-tetrayl)tetraacetic acid. In some embodiments, the metal chelator has a structure of

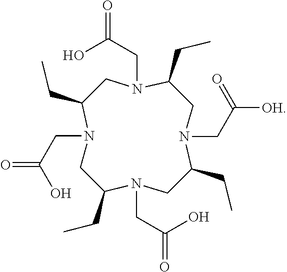 [SMILES: CC[C@H]1CN(CC(=O)O)[C@@H](CC)CN(CC(=O)O)[C@@H](CC)CN(CC(=O)O)[C@@H](CC)CN1CC(=O)O]

In some embodiments, the metal chelator has a structure of

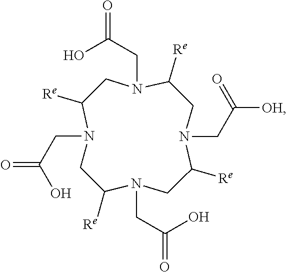

wherein each R^{c} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain. In some embodiments, the metal chelator has a structure of

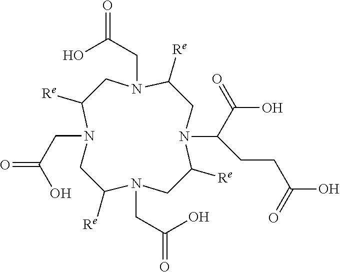

wherein each R^{c} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain.

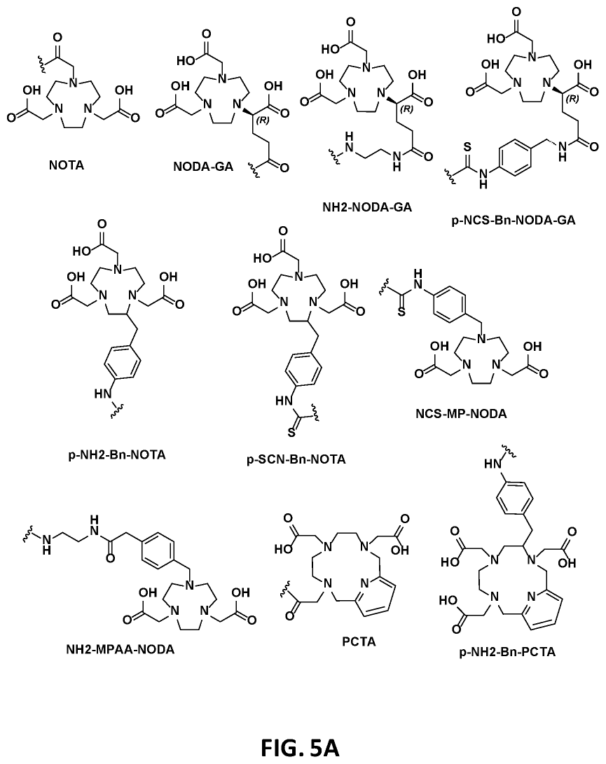

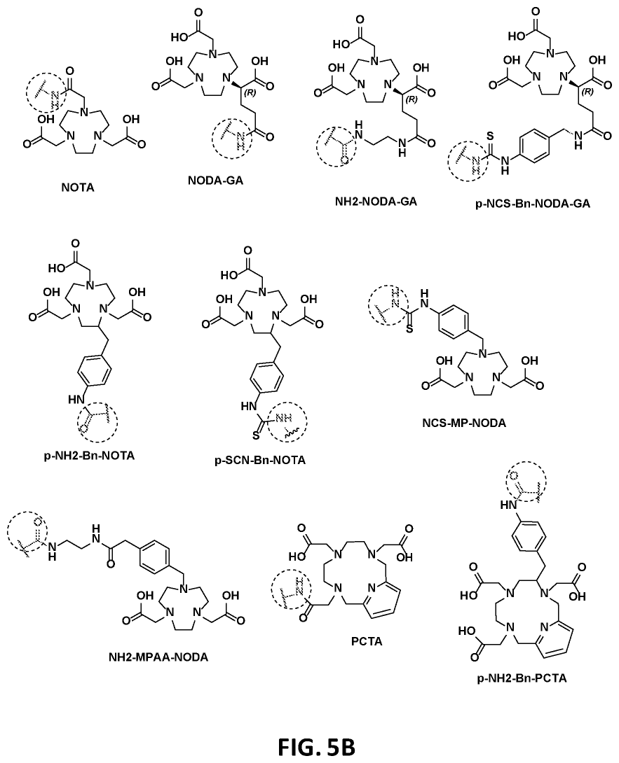

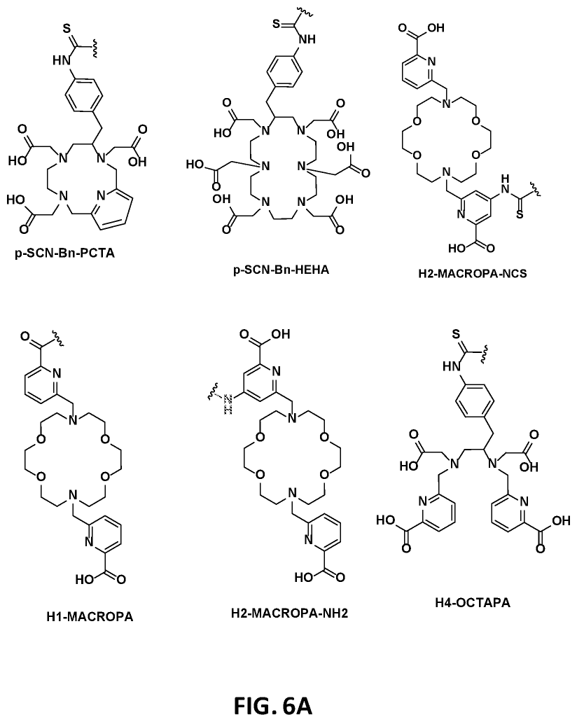

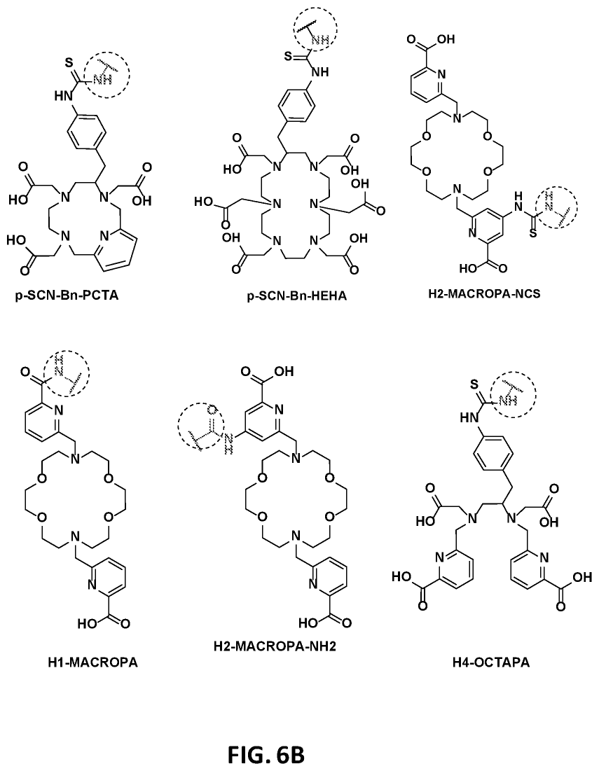

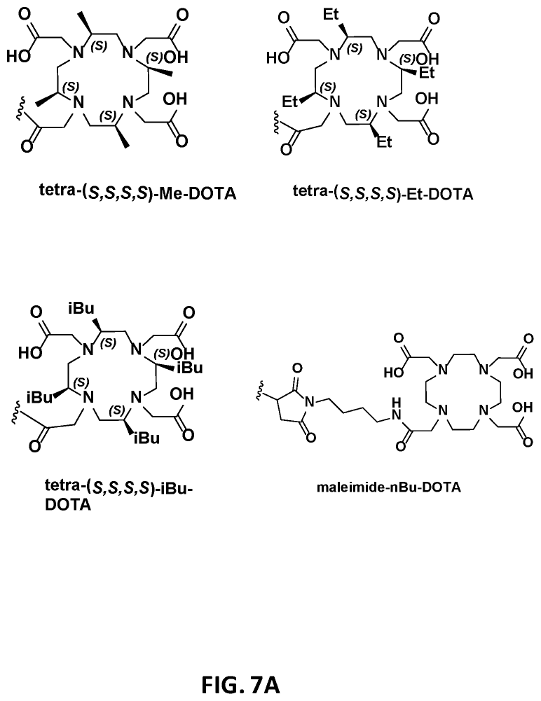

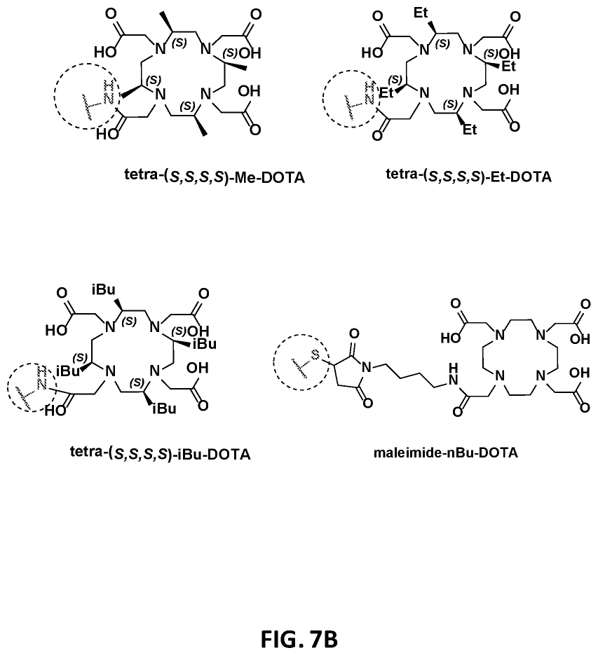

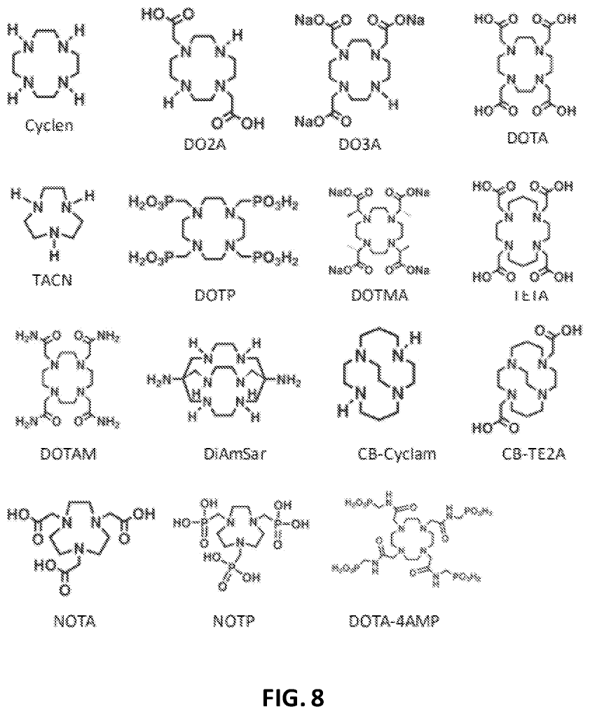

In some embodiments, the conjugate comprises DOTA. In some embodiments, the conjugate comprises a DOTA derivative such as p-SCN-Bn-DOTA and MeO-DOTA-NCS. In some embodiments, the conjugate comprises two independent metal chelators, and at least one or both are DOTA. The structures of some exemplary metal chelators are illustrated in FIGS. 8-22 (without showing the attachment points). Exemplary metal chelators are also illustrated in FIGS. 4A, 5A, 6A, and 7A (attachment point shown as a squiggly line) and FIGS. 4B, 5B, 6B and 7B (except that a part of the linker or the peptide covalently connected to the metal chelator is shown in the dashed circle). In some embodiments, a conjugate comprises a metal chelator of FIG. 4A. In some embodiments, a conjugate comprises a metal chelator of FIG. 5A. In some embodiments, a conjugate comprises a metal chelator of FIG. 6A. In some embodiments, a conjugate comprises a metal chelator of FIG. 7A. Exemplary metal chelators are further described in WO2012/174136; US20130183235A1; US20120219495A1; Ramogidaand et al., EJNMMI radiopharm. chem. 4, 21 (2019); Thiele et al., Cancer Biotherapy and Radiopharmaceuticals 2018; Li et al., Bioconjugate Chem. 2019, 30, 5, 1539-1553; and Baranyai et al., Eur. J. Inorg. Chem. 36-56 (2020), each of which is incorporated by reference in its entirety.

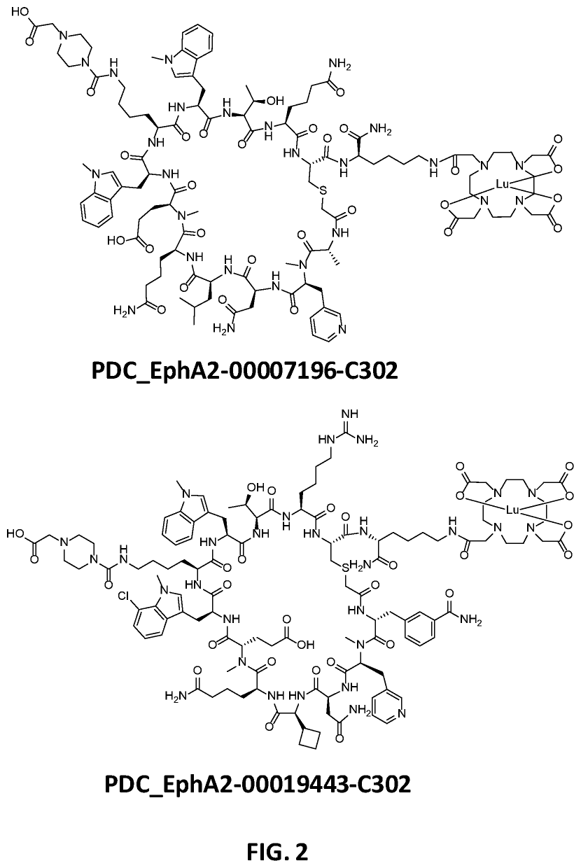

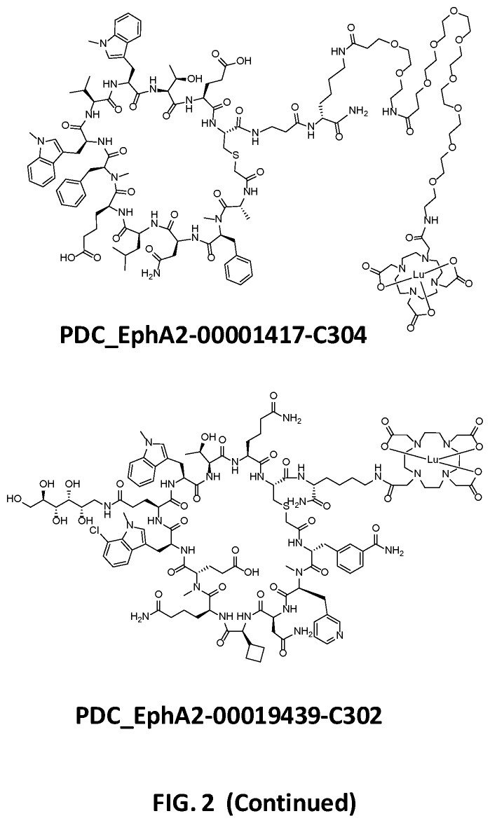

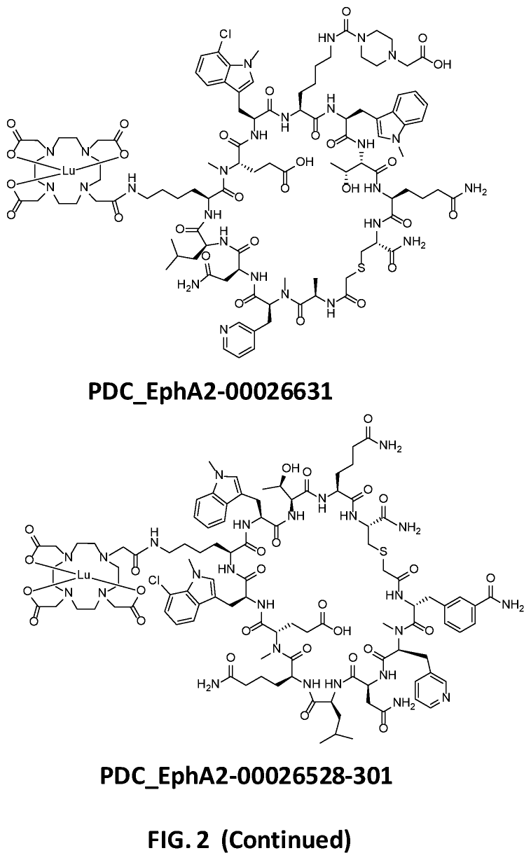

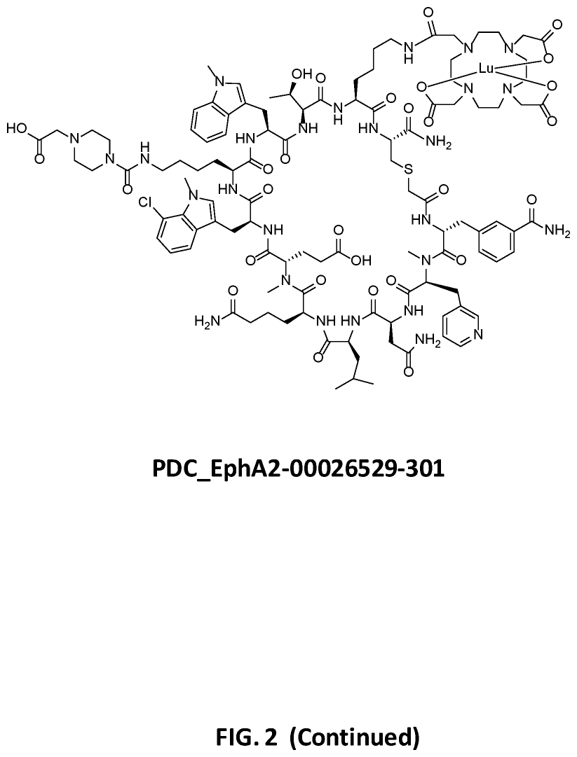

It is understood that the structures of conjugates in FIG. 2 are shown for illustration purposes. A person skilled in the art would appreciate that the bonding between the metal or radionuclide (La3+, ¹⁷⁷Lu or ²²⁵Ac) and the metal chelator in the conjugates is for illustration purpose.

A metal chelator such as DOTA can interact with a radionuclide (e.g., ¹⁷⁷Lu or ²²⁵Ac) via one or more functional groups and/or atoms. For example, a metal chelator can interact with a radionuclide via nitrogen and/or oxygen atoms. As another example, a metal chelator can interact with a radionuclide via carbonyl, carboxylic acid, amino, and/or amide groups of the metal chelator. In some embodiments, the interaction of a metal chelator and a radionuclide of the conjugates disclosed herein can be illustrated as

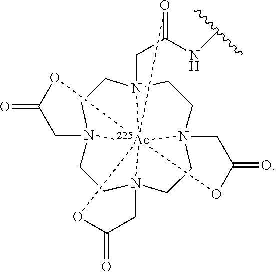

In some embodiments, the interaction of a metal chelator and a radionuclide of the conjugates disclosed herein can be illustrated as

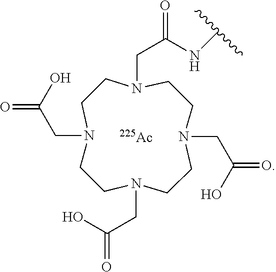

In some embodiments, the interaction of a metal chelator and a radionuclide of the conjugates disclosed herein can be illustrated as

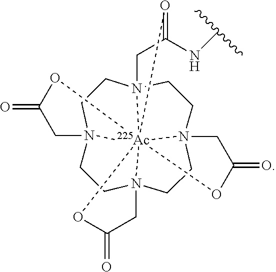

In some embodiments, the interaction of a metal chelator and a radionuclide of the conjugates disclosed herein can be illustrated as

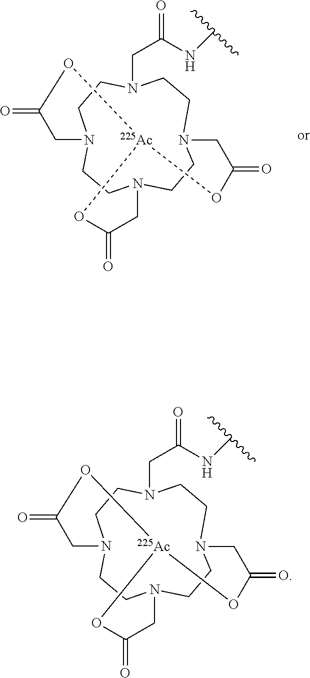

In some embodiments, the interaction of a metal chelator and a radionuclide of the conjugates disclosed herein can be illustrated as

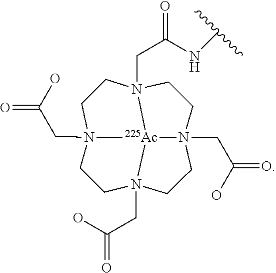

In some embodiments, the interaction of a metal chelator and a radionuclide of the conjugates disclosed herein can be illustrated as

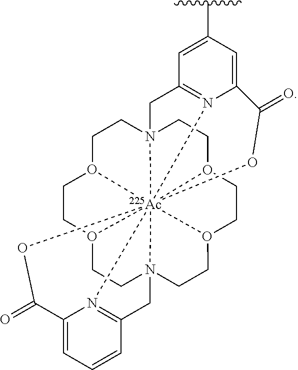

In some embodiments, the radionuclide exists in a positive oxidation state e.g., ²²⁵Ac³⁺, ¹⁷⁷Lu³⁺. In some embodiments, for example in certain aqueous conditions, the radionuclide exists in a salt form, e.g., as ²²⁵Ac³⁺, ¹⁷⁷Lu³⁺. In some embodiments, for example in certain acidic aqueous conditions, the radionuclide exists in a salt form, e.g., as ²²⁵Ac³⁺, ¹⁷⁷Lu³⁺. In some embodiments, the conjugate is in a salt form. In some embodiments, one or more of the carboxylic acid groups of the conjugate may exist as carboxylate anions. In some embodiments, one or more of the carboxylate anions of the conjugate may coordinate to the radionuclide. A person of ordinary skill would appreciate that the dissociation of an acid can depend on the pH value of the environment and its pK value. Accordingly, in some embodiments, a conjugate described herein can exist in a completely ionized, partially ionized or non-ionized form. Radionuclide

In one aspect, disclosed herein are radiopharmaceutical conjugates comprising a radionuclide. In some embodiments, the radionuclide is chelated or bound to a metal chelator. In some embodiments, the radionuclide is covalently bound to the conjugate. Generally, the type of radionuclide used in a therapeutic radiopharmaceutical can be tailored to the specific type of cancer, the type of targeting moiety (e.g., binding peptide), etc. Radionuclides that undergo a-decay produce particles composed of two neutrons and two protons, and radionuclides that undergo B-decay emit energetic electrons from their nuclei. Some radionuclides can also undergo electron capture and emit auger electrons. In some embodiments, the conjugate comprises an alpha particle-emitting radionuclide. Alpha radiation can cause direct, irreparable double-strand dna breaks compared with gamma and beta radiation, which can cause single-stranded breaks via indirect dna damage. The range of these particles in tissue and the half-life of the radionuclide can also be considered in designing the radiopharmaceutical conjugate. Table 7 below illustrates some properties of exemplary radionuclides.

TABLE 7
Exemplary radionuclides

| | | Exemplary | |
|---|---|---|---|
| | | application-- | |
| | | Therapeutics (Tx) | Chelator or |
| Radionuclide | Emission/Decay | or Diagnostics (Dx) | Covalent |
| Ac-225 | Alpha | Tx | Chelator |
| As-70 | Positron | Dx | Covalent |
| As-71 | Positron | Dx | Covalent |
| As-72 | Positron | Dx | Covalent |
| As-73 | Electron Capture | Dx | Covalent |
| As-74 | Positron | Dx | Covalent |
| As-76 | Beta | Tx | Covalent |
| As-77 | Beta | Tx | Covalent |
| At-211 | Alpha | Tx | Covalent |
| Bi-209 | Alpha | Tx | Chelator |
| Bi-212 | Alpha | Tx | Chelator |
| Bi-213 | Alpha | Tx | Chelator |
| Br-76 | Positron | Dx | Covalent |
| Ce-134 | Electron Capture | Dx | Chelator |
| Cu-61 | Positron | Dx | Chelator |
| Cu-62 | Positron | Dx | Chelator |
| Cu-64 | Positron | Dx | Chelator |
| Cu-67 | Beta | Tx | Chelator |
| Fr-223 | Alpha | Tx | Chelator |
| Ga-68 | Positron | Dx | Chelator |
| Gd-148 | Alpha | Tx | Chelator |
| Ho-166 | Beta/gamma | Tx/Dx | Chelator |
| I-123 | Electron Capture | Dx | Covalent |
| I-124 | Positron | Dx | Covalent |
| I-125 | Electron Capture | Dx | Covalent |
| I-131 | Beta | Tx | Covalent |
| In-111 | Beta/Electron | Tx/Dx | Chelator |
| | Capture | | |
| Lu-177 | Beta | Tx | Chelator |
| Pb-212 | Alpha | Tx | Chelator |
| Pm-153 | Beta | Tx | Chelator |
| Po-213 | Alpha | Tx | Chelator |
| Ra-223 | Alpha | Tx | Chelator |
| Ra-224 | Alpha | Tx | Chelator |
| Rh-105 | Beta | Tx | Chelator |
| Sm-153 | Beta | Tx | Chelator |
| Tb-149 | Alpha | Tx | Chelator |
| Tb-152 | Positron | Dx | Chelator |
| Tb-161 | Beta | Tx/Dx | Chelator |
| Tc-99m | Gamma/Isomeric | Dx | Chelator |
| | Transition | | |
| Th-227 | Alpha | Tx | Chelator |
| Th-229 | Alpha | Tx | Chelator |
| Tm-167 | Beta/Electron | Tx | Chelator |
| | Capture | | |
| Y-90 | Beta | Tx | Chelator |
| Yb-175 | Beta | Tx | Chelator |
| Zr-89 | Positron | Dx | Chelator |

In some embodiments, the radiopharmaceutical conjugate described herein comprises a radionuclide selected from Table 7.

In some embodiments, the radiopharmaceutical conjugate described herein comprises one or more independent radionuclides. In some embodiments, the radiopharmaceutical conjugate comprises two radionuclides. In some embodiments, each of the one or more radionuclides is bound to the metal chelator of the radiopharmaceutical conjugate. In some embodiments, two radionuclides of the radiopharmaceutical conjugate are bound to the same metal chelator. In some embodiments, two radionuclides of the radiopharmaceutical conjugate are bound to two independent metal chelators. In some embodiments, each of the one or more radionuclides is an alpha particle-emitting radionuclide.

In some embodiments, the radiopharmaceutical conjugate described herein comprises an alpha particle-emitting radionuclide. In some embodiments, the radiopharmaceutical conjugate comprises an alpha-particle emitting radionuclide bound to the metal chelator. In some embodiments, the alpha particle-emitting radionuclide is actinium-225 (225Ac), radium-223 (²²³Ra), radium-224 (²²⁴Ra), bismuth-209 (²⁰⁹Bi), bismuth-213 (²¹³Bi), gadolinium-148 (¹⁴⁸Gd), terbium-149 (¹⁴⁹Tb), polonium-213 (213Po), francium-223 (²²³Fr), thorium-227 (²²⁷Th), thorium-229 (²²⁹Th), or lead-212 (²¹²Bb). In some embodiments, the alpha particle-emitting radionuclide is selected from ²²⁵Ac, ²²³Ra, ²⁰⁹Bi, ²¹³Bi, ¹⁴⁸Gd, ¹⁴⁹Tb, ²¹³Po, ²²³Fr, ²²⁷Th, ²²⁹Th, and ²¹²Pb. In some embodiments, the alpha particle-emitting radionuclide is ²²⁵Ac. In some embodiments, the alpha particle-emitting radionuclide is 213Bi. In some embodiments, the alpha particle-emitting radionuclide is ²¹²Bi. In some embodiments, the alpha particle-emitting radionuclide is ²¹²Pb. In some embodiments, the alpha particle-emitting radionuclide is ²²⁴Ra. In some embodiments, the alpha particle-emitting radionuclide is ²²³Ra. In some embodiments, the alpha particle-emitting radionuclide is ²²⁷Th. In some embodiments, the alpha particle-emitting radionuclide is ¹⁴⁹Tb. In some embodiments, the conjugate comprises ²²⁵Ac. In some embodiments, the conjugate comprises two ²²⁵Ac radionuclides. In some embodiments, the radionuclide is no-carrier added (i.e., non-carrier-added or n.c.a.) ¹⁷⁷Lu. In some embodiments, the radionuclide is no-carrier added (i.e., non-carrier-added or n.c.a.) ²²⁵Ac. In some embodiments, the radionuclide is ¹⁷⁷Lu free of long-lived radioactive contaminants and byproducts. In some embodiments, the conjugate comprises two ¹⁷⁷Lu radionuclides. In some embodiments, the radionuclide is a non-carrier-added radionuclide. In some embodiments, the radionuclide is a pseudo-radiometal. In some embodiments, the pseudo-radiometal is aluminum-[18F]fluoride ([18F]AIF) complex.

In some embodiments, the radiopharmaceutical conjugate described herein comprises a radionuclide selected from ⁶²Cu, ⁶⁴Cu, ⁶⁷Cu, ⁹⁰Y, ¹⁰⁹Pd, ¹¹¹Ag, ¹³⁴Ce, ¹⁴⁹Pm, ¹⁵³Sm, ¹⁶⁶Ho, ^{99m}Tc, ⁶⁷Ga, ⁶⁸Ga, ¹¹¹In, ⁹⁰Y, ¹⁷⁷Lu, ¹⁸⁶Re, ¹⁸⁸Re, ¹⁹⁷Au, ¹⁹⁸Au, ¹⁹⁹Au, ¹⁰⁵Rh, ¹⁶⁵Ho, ¹⁶¹Tb, ¹⁴⁹Pm, ¹⁵³Pm, ⁴⁴Sc, ⁴⁷Sc, ²¹³Po, ²¹²Pb, ²⁰⁹Bi, ²¹²Bi, ²¹³Bi, ²²⁵Ac, ^{117m}Sn, ⁶⁷Ga, ¹⁴⁹Tb, ¹⁵²Tb, ¹⁶⁷Tm, ¹⁷⁵Yb, ²²³Ra, ²²³Fr, ²²⁷Th, ²²⁹Th, ²⁰¹Tl, ¹⁴⁸Gd, ¹⁶⁰Gd, ¹⁴⁸Nd, ⁸⁹Sr, and ⁸⁹Zr. In some embodiments, the radionuclide is selected from ⁶²Cu, ⁶⁴Cu, ⁶⁷Cu, ⁶⁸Ga, ⁸⁹Zr, ⁹⁰Y, ⁹⁹mTc, ¹⁰⁵Rh, ¹¹¹In, ¹³⁴Ce, ¹⁴⁸Gd, ¹⁴⁹Tb, ¹⁵²Tb, ¹⁵³Pm, ¹⁶⁷Tm, ¹⁷⁵Yb, ¹⁷⁷Lu, ²⁰⁹Bi, ²¹²Pb, ²¹³Po, ²¹³Bi, ²²³Ra, ²²³Fr, ²²⁷Th, ²²⁵Ac, and ²²⁹Th. In some embodiments, the radionuclide is ²²⁵Ac. In some embodiments, the radionuclide is a decay daughter of ²²⁵Ac such as ²²¹Fr, ²¹⁷At, ²¹³Bi, ²¹³Po, ²⁰⁹Tl, ²⁰⁹Pb, or ²⁰⁹Bi. In some embodiments, the radiopharmaceutical conjugate comprises two ²²⁵Ac radionuclides. In some embodiments, the radionuclide is ¹⁷⁷Lu. In some embodiments, the radiopharmaceutical conjugate comprises two ¹⁷⁷Lu radionuclides.

In some embodiments, the radiopharmaceutical conjugate described herein comprises a beta particle-emitting radionuclide. In some embodiments, the radiopharmaceutical conjugate comprises a beta particle-emitting radionuclide bound to the metal chelator. In some embodiments, the beta particle-emitting radionuclide is copper-67, rhodium-105, ytterbium-175, thulium-167, promethium-153, yttrium-90, samarium-153, or lutetium-177. In some embodiments, the beta particle emitting radionuclide is copper-67, yttrium-90, samarium-153, or lutetium-177. In some embodiments, the beta particle emitting radionuclide is lutetium-177.

In some embodiments, the radiopharmaceutical conjugate described herein comprises a gamma particle-emitting radionuclide. In some embodiments, the radiopharmaceutical conjugate comprises a gamma particle-emitting radionuclide bound to the metal chelator. In some embodiments, the gamma particle-emitting radionuclide is indium-111 or tin-117m.

In some embodiments, the radiopharmaceutical conjugate described herein comprises a positron particle-emitting radionuclide. In some embodiments, the radiopharmaceutical conjugate comprises a positron particle-emitting radionuclide bound to the metal chelator. In some embodiments, the positron-emitting radionuclide is gallium-68, copper-61, copper-62, copper-64, zirconium-89, or terbium-152. In some embodiments, the radionuclide is zirconium-89. In some embodiments, the radionuclide is gallium-68.

In some embodiments, a conjugate described herein comprises a radionuclide suitable for imaging or diagnostic purposes. In some embodiments, the radionuclide suitable for imaging is selected from ⁶²Cu, ⁶⁴Cu, ⁸⁹Zr, ¹³⁴Ce, ¹⁵²Tb, ⁶⁸Ga, ¹¹¹In, and ^{99m}Tc. In some embodiments, the radionuclide is suitable PET imaging. In some embodiments, the radionuclide suitable for PET imaging is selected from ⁶²Cu, ⁶⁴Cu, ⁸⁹Zr, ¹³⁴Ce, ¹⁵²Tb, and ⁶⁸Ga. In some embodiments, the radionuclide is suitable for SPECT imaging. In some embodiments, the radionuclide suitable for SPECT imaging is selected from ¹¹¹In and ^{99m}Tc.

In some embodiments, radiopharmaceutical conjugates described herein do not contain any hot radionuclide, i.e., a cold conjugate. For example, in some cases, a radionuclide can be replaced with a surrogate (e.g., ²²⁵Ac replaced with lanthanum) for testing and experimental purposes. In some embodiments, hot lutetium (Lu-177) is replaced with a cold lutetium (Lu-175).

In some embodiments, a radiopharmaceutical conjugate described herein comprises a covalently bound radionuclide and optionally a linker. In some embodiments, the linker can comprise a residualizing agent or a non-residualizing agent. A radionuclide can be attached to a peptide or a linker through a residualizing agent or a non-residualizing agent. In some embodiments, the radionuclide is covalently attached to the peptide or the linker through a residualizing agent. In some embodiments, the residualizing agent is SGMIB or SIPC. In some embodiments, the radionuclide is covalently attached to the peptide or the linker through a residualizing agent. In some embodiments, the non-residualizing agent is N-succinimidyl-4-iodobenzoate (PIB). In some embodiments, a radionuclide is covalently bound to the residualizing agent or the non-residualizing agent. In some embodiments, the radionuclide is covalently bound to the residualizing agent. Procedures and methods for synthesis of covalently bound residualizing agents are described in U.S. Pat. No. 9,839,704 which is herein incorporated by reference in its entirety.

In some embodiments, the residualizing agent is a tetrapeptide IMP-R4. IMP-R4 can be represented as MCC-Lys(MCC)-Lys(Z)-d-Tyr-d-Lys(Z)-OH (SEQ ID NO: 415), where MCC is 4-(N-maleimidomethyl)-cyclohexane-1-carbonyl and Z is 1-((4-thiocarbonylamino)benzyl)-DTPA. In some embodiments, the radionuclide ¹³¹I is linked to linker or peptide via 131I-IMP-R. In some embodiments, the residualizing agent is a tetrapeptide IMP-R3. In some embodiments, the residualizing agent is a tetrapeptide IMP-R5. In some embodiments, the residualizing agent is a tetrapeptide IMP-R6. In some embodiments, the residualizing agent is a tetrapeptide IMP-R7. In some embodiments, the residualizing agent is a tetrapeptide IMP-R8. Exemplary residualizing and non-residualizing agents are further illustrated in Stein R, et al. Improved iodine radiolabels for monoclonal antibody therapy, Cancer Res. 2003; 63:111-118; Reist C J, et al. Radioiodination of internalizing monoclonal antibodies using N-succinimidyl-5-iodo-3-pyridinecarboxylate, Cancer Res. 1996; 56:4970-4977; Ali S A, et al. Improving the tumor retention of radioiodinated antibody: aryl carbohydrate adducts. Cancer Res. 1990; 50 (suppl): 783s-788s; and Serengulam V. Govindan, et al., Clinical-Scale Radiolabeling of a Humanized Anticarcinoembryonic Antigen Monoclonal Antibody, hMN-14, with Residualizing 131I for Use in Radioimmunotherapy, Journal of Nuclear Medicine January 2005, 46 (1) 153-159.

In some embodiments of a radiopharmaceutical conjugate of the present disclosure (e.g., conjugates of Formula (III-1-RI) or Formula (III-2-RI)), the conjugate comprises a covalently bound radionuclide, designated as R*, wherein R* is connected to the rest of the conjugate via a structure of

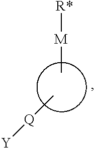

wherein

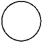

is a cycloalkyl, heterocycloalkyl, aryl, or heteroaryl ring, each of which is optionally substituted;
Y is a charged group;
M and Q are chemical linking moieties; and
R* is a covalently bound radioisotope (e.g., ¹⁸F, ¹²³I, ¹²⁴I, ¹²⁵I, ¹³¹I, and ²¹¹At).

In some embodiments,

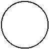

is phenyl.

In some embodiments, M and Q is each independently bond, C₁₋₂₀ alkylene, C₁₋₂₀ alkenylene, C₁₋₂₀ alkynylene, or C₁₋₂₀ heteroalkylene, wherein the alkylene, alkenylene, alkynylene, and heteroalkylene are optionally substituted with C₁₋₈ alkyl or C₃₋₇ cycloalkyl. In some embodiments, M is a bond. In some embodiments, Q is a bond or C₁ alkylene.

In some embodiments, Y is a guanidino group.

In some embodiments,

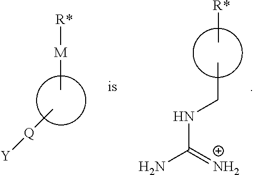

In some embodiments,

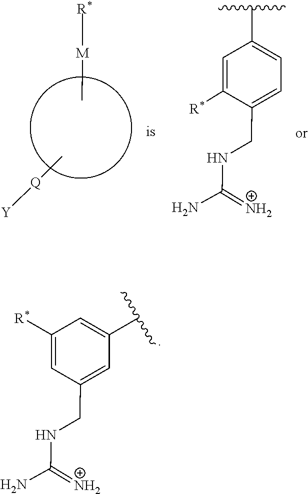

In some embodiments,

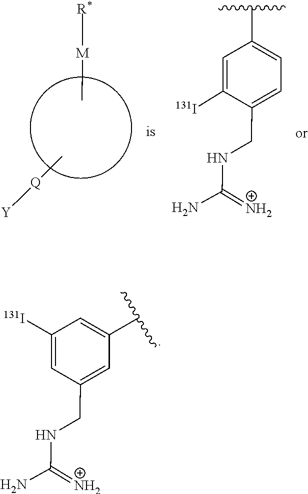

In some embodiments, R* is connected through the rest of the conjugate via a linker of the following structures

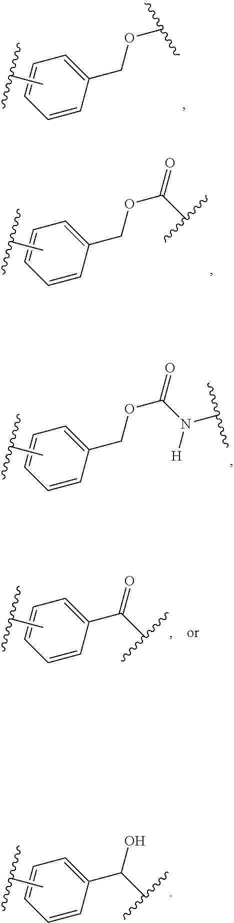

In some embodiments, a linker of the present disclosure, or one of L, L¹, L², L³, L⁴, or L⁵, comprises a structure selected from the group consisting of

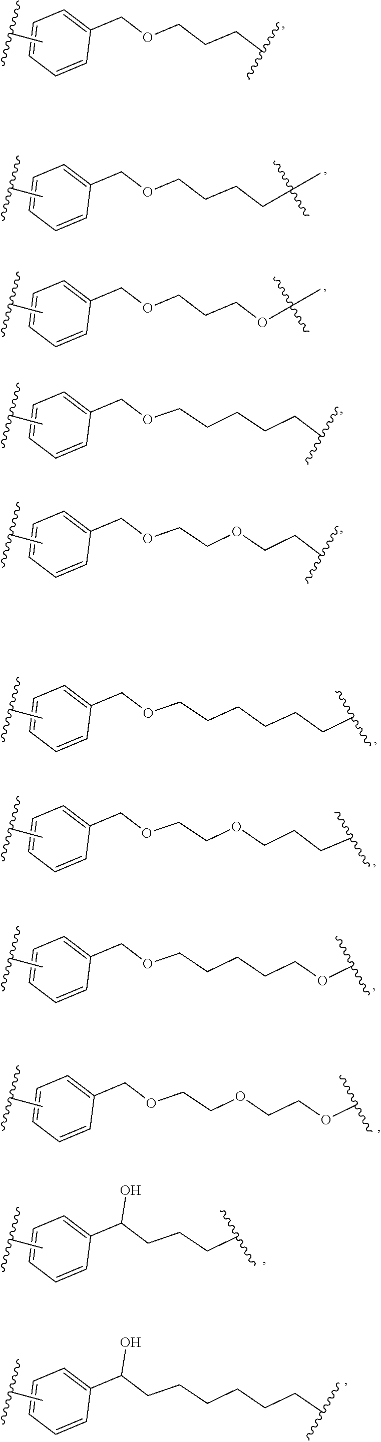

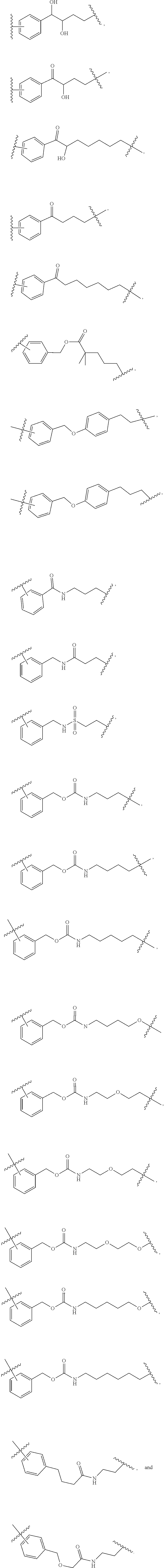

In some embodiments, the covalent radionuclide R* is connected to the phenyl.

In some embodiments, R* is selected from a radioisotope in Table 7. In some embodiments, the radioisotope is selected from fluorine-18 (¹⁸F), iodine-131 (¹³¹I), iosine-123 (¹²³I), iodine-124 (¹²⁴I), iodine-125 (¹²⁵I), or astatine-211 (²¹¹At). In some embodiments, R* is iodine-131 (¹³¹I) or astatine-211 (²¹¹At). In some embodiments, the radioisotope is ¹³¹I. In some embodiments, the radioisotope is ¹²⁴I. In some embodiments, the radioisotope is ¹²⁵I. In some embodiments, the radioisotope is ²¹¹At.

In some embodiments, a conjugate comprises a structure represented by Formula (Va), Formula (Vb), Formula (Vc), Formula (Vd), or Formula (Ve):

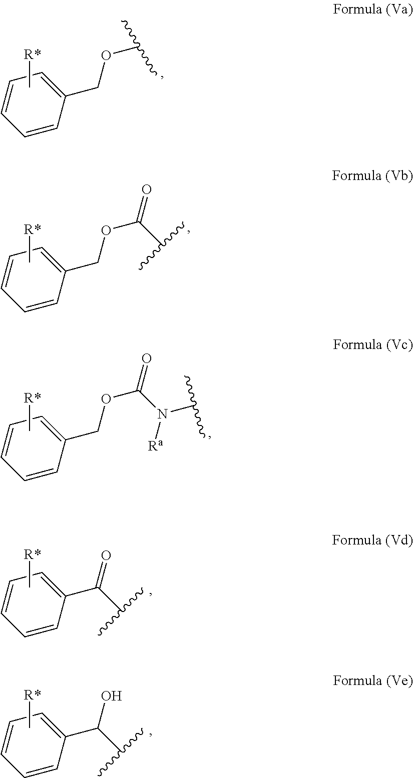

wherein,
   R* is a covalently bound radioisotope (e.g., such as fluorine-18 (¹⁸F), iodine-131 (¹³¹I), iodine-123 (¹²³I), iodine-124 (124I), iodine-125 (125I), or astatine-211 (²¹¹At)); and
   R^{a} is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₆heterocycloalkyl, aryl, or heteroaryl, wherein each of the alkyl, heteroalkyl, alkenyl, alkynyl, cycloalkyl, heterocycloalkyl, aryl, or heteroaryl is optionally substituted (e.g., optionally substituted with one or more R as described herein).

As illustrated above, Formula (Va), Formula (Vb), Formula (Vc), Formula (Vd), and Formula (Ve) can comprise all or a part of a linker and the radioisotope R*.

In some embodiments of Formula (Va), Formula (Vb), Formula (Vc), Formula (Vd), or Formula (Ve), the alkyl, haloalkyl, hydroxyalkyl, aminoalkyl, heteroalkyl, alkenyl, alkynyl, cycloalkyl, heterocycloalkyl, aryl, or heteroaryl are independently optionally substituted by one or more halogen, amino, —OH, —NO₂, oxo, —CN, C₁₋₃ alkoxyl, C₁₋₃ alkyl and C₁₋₃ haloalkyl. In some embodiments, R^{a} is hydrogen. In some embodiments, R^{a} is C₁-C₄alkyl. In some embodiments, R^{a} is C₁-C₄cycloalkyl. In some embodiments, R* is ¹³¹I.

In some embodiments, a structure represented in Formula (Va), Formula (Vb), Formula (Vc), Formula (Vd), and Formula (Ve) is selected from the following:

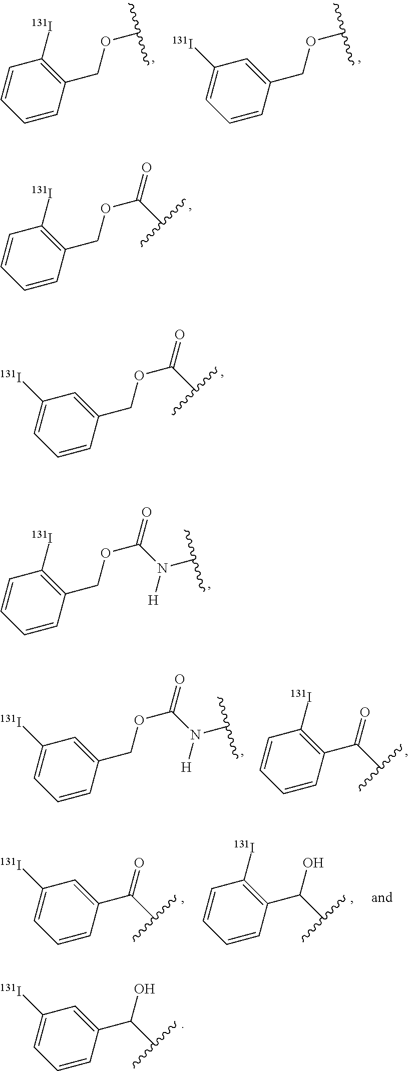

In some embodiments, a conjugate comprises a linker structure selected from:

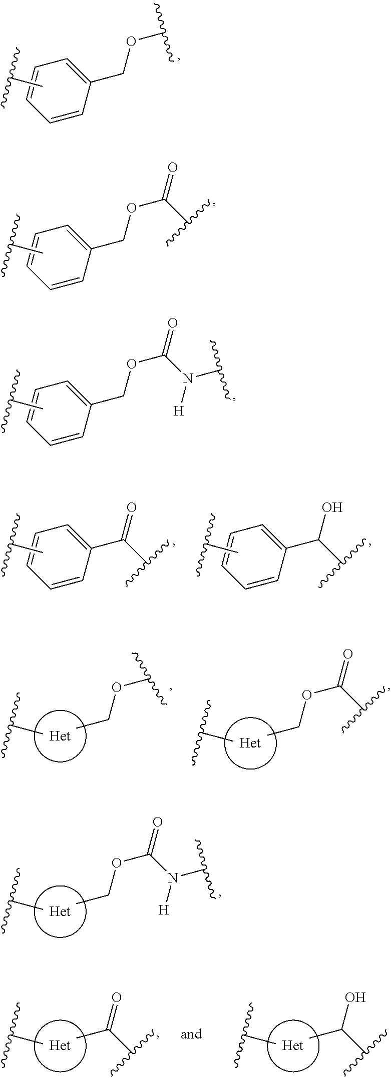

wherein Het is a 5-6 membered heteroaryl ring containing 1-3 heteroatoms independently selected from N, S, and O. In some embodiments, Het is pyridinyl or pyrimidinyl.

In some embodiments, a conjugate comprises a linker structure selected from:

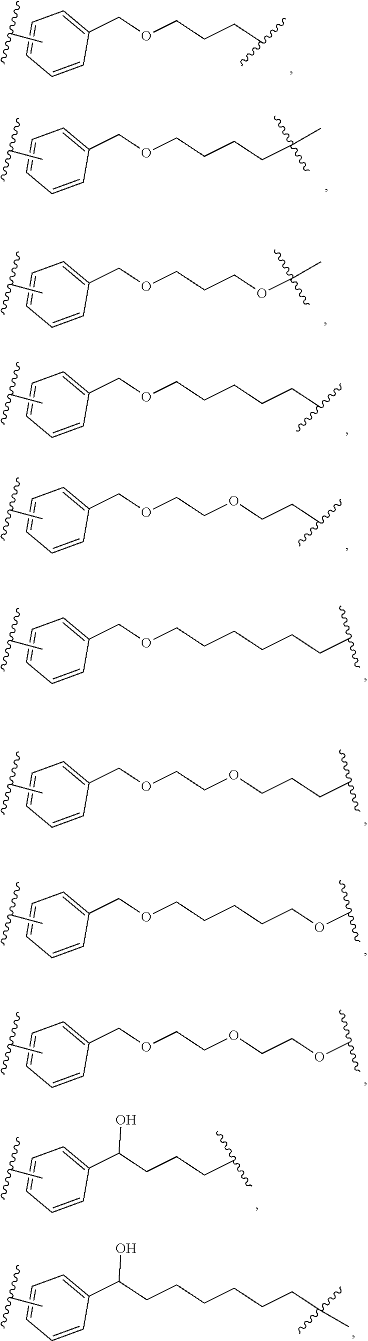

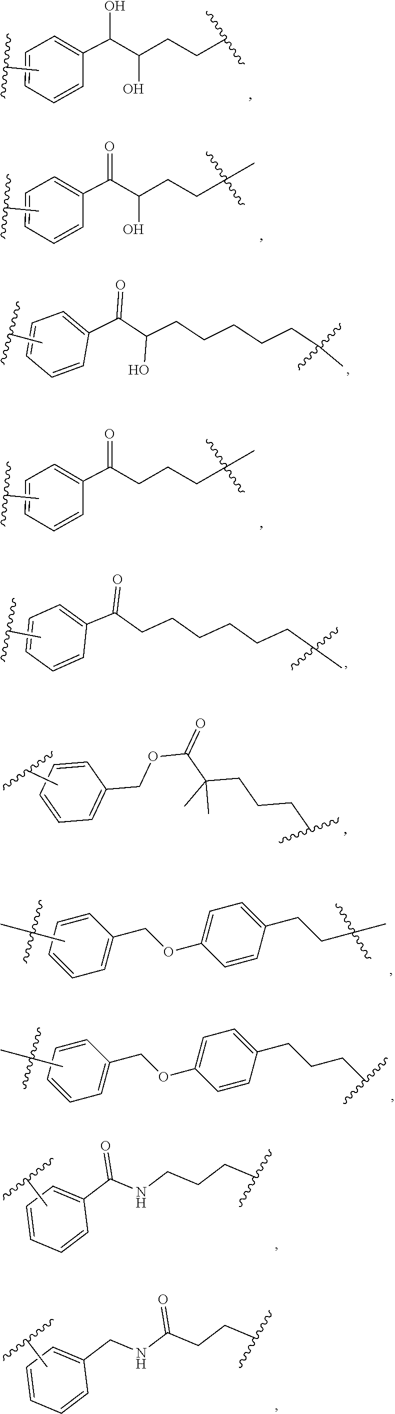

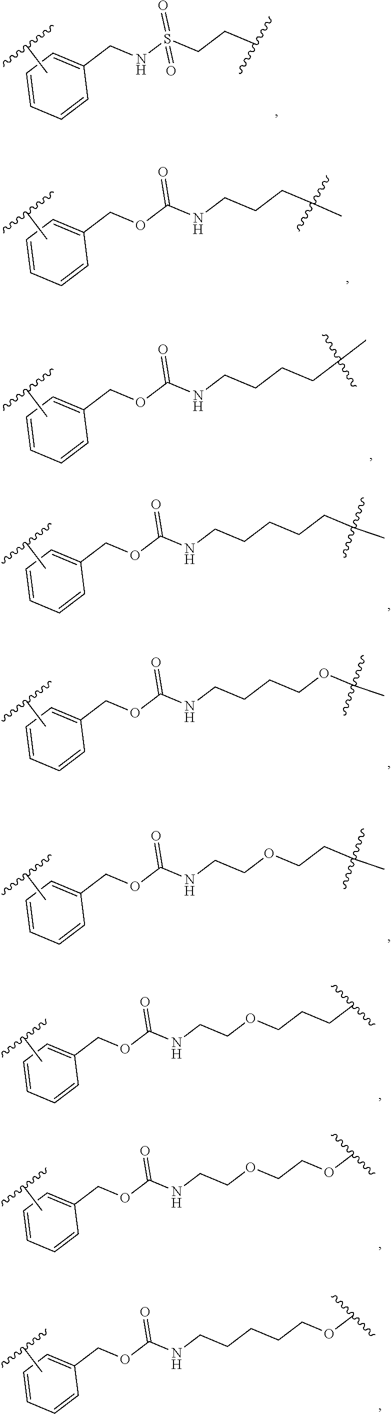

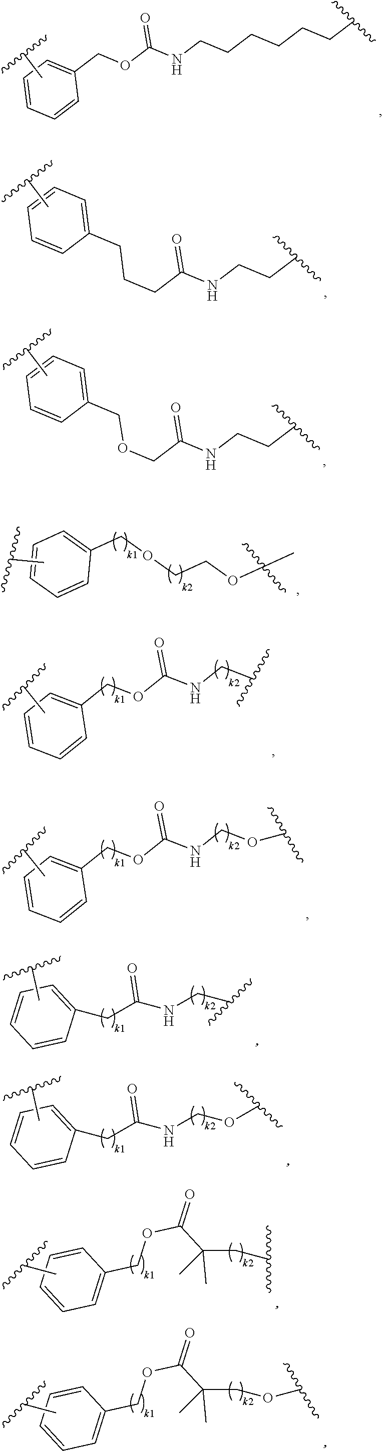

wherein each k1 and k2 is independently 0 or an integer selected from 1 to 10. In some embodiments, R* is attached to phenyl.

In some embodiments, a conjugate described herein comprises one or more independent radionuclides. In some embodiments, the conjugate comprises two radionuclides. In some embodiments, each of the one or more radionuclides is an alpha particle-emitting radionuclide. In some embodiments, each of the one or more radionuclides is a beta particle-emitting radionuclide.

In some embodiments, a radiolabeled conjugate described herein comprises an alpha particle-emitting radionuclide. In some embodiments, the alpha particle-emitting radionuclide is astatine-211 (²¹¹At).

In some embodiments, the conjugate comprises a covalently bound beta particle-emitting radionuclide. In some embodiments, the beta particle emitting radionuclide is iodine-131 (¹³¹I).

In some embodiments, the conjugate comprises a covalently bound β+ positron-emitting radionuclide. In some embodiments, the β+ positron emitting radionuclide is fluorine-18 (¹⁸F).

In some embodiments, the conjugate comprises a gamma particle emitting radionuclide. In some embodiments, the gamma particle emitting radionuclide is iodine-123.

In some embodiments, provided herein are conjugates having the structures of the radiolabeled conjugates described herein, except that the covalent radionuclide is replaced with a surrogate (e.g., ¹³¹I replaced with iodine), i.e., a cold conjugates. In some embodiments, a covalent radionuclide of the radiolabeled conjugates described herein can be replaced with a surrogate (e.g., ¹³¹I replaced with iodine) for testing and experimental purposes.

In some embodiments, radiopharmaceutical conjugates comprising covalently bound radionuclides described herein can be synthesized from a boronic acid, boronate, or stannane precursor. Boronic acid, boronate, and stannane precursors can be formed according to the following general reaction:

### Reaction 1:

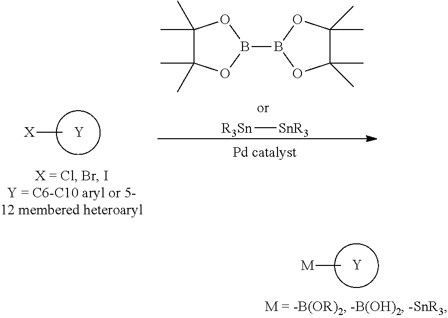

where R is a variable chemical moiety, for example alkyl or hydrogen. A halogen on ring Y, for example chloro, bromo, or iodo, can undergo a palladium mediated coupling reaction with a boronic acid, boronate, or stannane compound to form an intermediate compound wherein group M has replaced halogen X.

A covalent radionuclide, for example R*, such as fluorine-18 (¹⁸F), iodine-131 (¹³¹I), iodine-123 (¹²³I), iodine-124 (124I), iodine-125 (125I), or astatine-211 (²¹¹At), or any other suitable radioisotope of Table 7 can be formed, for example, from the intermediate compound according to the following general reactions:

### Reaction 2:

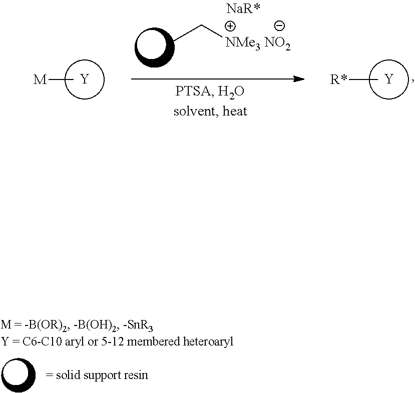

### Reaction 3:

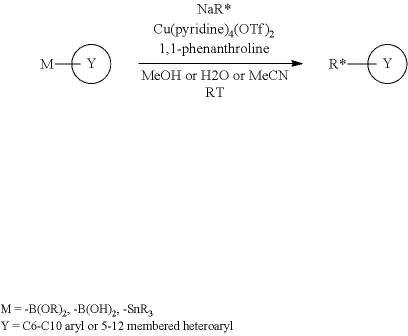

### Reaction 4:

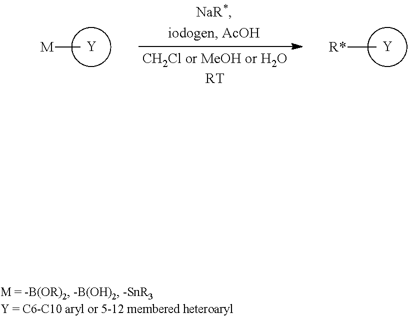

where R is a variable chemical moiety, for example alkyl or hydrogen.

In some embodiments, radiolabeled conjugates described herein can be synthesized from a chloro, bromo, or iodo precursor according to the following general reaction:

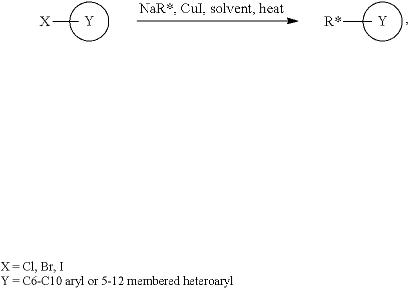

where R* is a radioisotope, for example, fluorine-18 (¹⁸F), iodine-131 (¹³¹I), iodine-123 (¹²³I), iodine-124 (124I), iodine-125 (125I), astatine-211 (²¹¹At), or a radioisotope of Table 7 marked “Covalent”.

The radiolabeling reactions depicted above are used as example procedures in the synthesis of radiolabeled conjugates described herein. Additional reactions, including nucleophilic substitution, electrophilic substitution, isotopic exchanges, bromine-radioiodine exchange, radioiododestannylation, radioiododeboronation, and transition metal mediated halogen exchange are contemplated and procedures can be found in Berdal et al., “Investigation on the reactivity of nucleophilic radiohalogens with arylboronic acids in water: access to an efficient single-step method for the radioiodination and astatination of antibodies” Chemical Science 2021, 12, 1458, and Dubost et al., “Recent Advances in Synthetic Methods for Radioiodination” J. Org. Chem, 2020, 85, 13, 8300-8310, both of which are incorporated by reference herein in their entirety.

Accordingly, in one aspect, described herein is a method of synthesizing a radiopharmaceutical conjugate or salt or solvate or pharmaceutical composition thereof. In some embodiments, the method comprises replacing a halogen on a precursor conjugate with a radioisotope, e.g., a fluorine-18 (¹⁸F), iodine-131 (¹³¹I), iodine-123 (¹²³I), iodine-124 (124I), iodine-125 (125I), astatine-211 (²¹¹At), or a suitable radioisotope of Table 7. In some embodiments, the method comprises a transition metal (e.g., palladium) mediated coupling reaction with a boronic acid, boronate, or stannane compound to form an intermediate compound. In some embodiments, the method further comprises exchanging the boronic acid, boronate, or stannane group with the radioisotope, e.g., as illustrated above. In some embodiments, the method comprises nucleophilic substitution, electrophilic substitution, isotopic exchanges, bromine-radioiodine exchange, radioiododestannylation, radioiododeboronation, and/or transition metal mediated halogen exchange reactions.

In some embodiments, a radiopharmaceutical conjugate disclosed herein comprises a pseudo-radiometal, for example, an aluminum-18F complex. In some embodiments, the aluminum-18F complex is bound to a metal chelator.

### Isomers/Stereoisomers

In some embodiments, the compounds described herein exist as geometric isomers. In some embodiments, the compounds described herein possess one or more double bonds. The compounds presented herein include cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers

A “tautomer” refers to a molecule wherein a proton shift from one atom of a molecule to another atom of the same molecule is possible. The conjugates presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

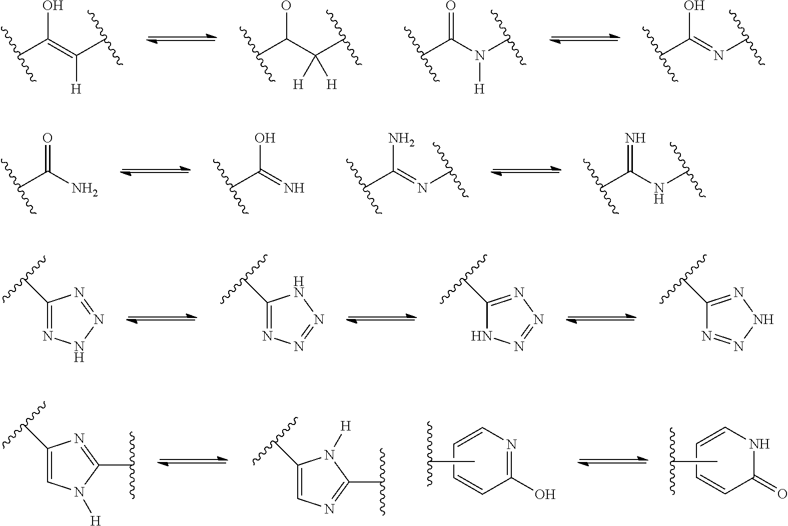

In some instances, the conjugates disclosed herein exist in tautomeric forms. The structures of said conjugates are illustrated in the one tautomeric form for clarity. The alternative tautomeric forms are expressly included in this disclosure.

### Labeled Compounds

In some embodiments, the conjugates described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled conjugates as pharmaceutical compositions. For example, the conjugates described herein may be artificially enriched in one or more particular isotopes. In some embodiments, the conjugates described herein may be artificially enriched in one or more isotopes that are not predominantly found in nature. In some embodiments, the conjugates described herein may be artificially enriched in one or more isotopes selected from deuterium (²H), tritium (³H), and/or carbon-14 (¹⁴C). All isotopic variations of the conjugates of the present disclosure are encompassed within the scope of the present disclosure. Examples of isotopes that can be incorporated into conjugates described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Conjugates described herein, and the pharmaceutically acceptable salts, solvates, or stereoisomers thereof which contain the aforementioned isotopes and/or other isotopes of other atoms are within the scope of this disclosure. Certain isotopically-labeled conjugates, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are notable for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled conjugate or a pharmaceutically acceptable salt, solvate, or stereoisomer thereof is prepared by any suitable method.

In some embodiments, the conjugates described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions. As used herein, a “pharmaceutically acceptable salt” refers to any salt of a compound that is useful for therapeutic purposes of a subject.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral acid, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. This disclosure provides for methods of treating diseases by administering such solvates. This disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Accordingly, one aspect of the present disclosure pertains to hydrates and solvates of compounds of the present disclosure and/or their pharmaceutical acceptable salts, as described herein, that can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like.

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

In one aspect, described herein is a method of making a conjugate that comprises a cyclic peptide, a metal chelator, optionally a linker, and optionally a radionuclide such as 177Lu or 225Ac. In some embodiments, the conjugate is prepared by one or more of the following steps: (a) synthesizing the peptide sequence by solid phase peptide synthesis; (b) cyclizing the peptide by forming an intramolecular non-peptide bond; (c) coupling the metal chelator to the peptide; (d) and optionally labeling the conjugate with a radionuclide. In some embodiments, steps (a), (b), (c) and (d) are performed in the recited order. In some embodiments, synthesizing the peptide comprises synthesizing the peptide sequence in a protected form and performing a de-protecting reaction. In some embodiments, cyclizing the peptide comprises forming a non-peptide bond between the N-terminus and the C-terminus of the peptide. In some embodiments, cyclizing the peptide comprises forming a non-peptide bond between the N-terminus and a cysteine or homocysteine of the peptide. In some embodiments, cyclizing the peptide comprises forming a ring closing group selected from —C(═O)—CH₂—, —C(═O)—CH₂—S—, —S—, —CH═CH—, —NH—, -maleimide-S—, —C(═O)—CH₂—NH—, and —C(═O)—CH₂—O—. In some embodiments, cyclizing the peptide comprises forming a ring closing group of Table 4B. In some embodiments, cyclizing the peptide comprises reacting a pair of functional groups or amino acids described in Table 4C. In some embodiments, solid phase peptide synthesis can be replaced with other suitable peptide synthesis methods known in the art.

In another aspect, described herein is a method of making a conjugate that comprises a cyclic peptide, optionally a linker, and optionally a covalent radionuclide such as ¹³¹I or ²¹¹At. In some embodiments, the conjugate is prepared by one or more of the following steps: (a) synthesizing the peptide sequence by solid phase peptide synthesis; (b) cyclizing the peptide by forming an intramolecular non-peptide bond; (c) optionally coupling the linker to the peptide; (d) and labeling the conjugate with a radionuclide. In some embodiments, steps (a), (b), (c) and (d) are performed in the recited order. In some embodiments, synthesizing the peptide comprises synthesizing the peptide sequence in a protected form and performing a de-protecting reaction. In some embodiments, cyclizing the peptide comprises forming a non-peptide bond between the N-terminus and the C-terminus of the peptide. In some embodiments, cyclizing the peptide comprises forming a non-peptide bond between the N-terminus and a cysteine or homocysteine of the peptide. In some embodiments, cyclizing the peptide comprises forming a ring closing group selected from —C(═O)—CH₂—, —C(═O)—CH₂—S—, —S—, —CH═CH—, —NH—, -maleimide-S—, —C(═O)—CH₂—NH—, and —C(═O)—CH₂—O—. In some embodiments, cyclizing the peptide comprises forming a ring closing group of Table 4B. In some embodiments, cyclizing the peptide comprises reacting a pair of functional groups or amino acids described in Table 4C. In some embodiments, solid phase peptide synthesis can be replaced with other suitable peptide synthesis methods known in the art.

### III. PHARMACEUTICAL COMPOSITIONS

The radiopharmaceutical conjugate described herein, including e.g., pharmaceutically acceptable salt or solvate thereof, can be administered per se as a pure chemical or as a component of a pharmaceutically acceptable formulation. In some embodiments, a conjugate described herein is combined with a pharmaceutically suitable or acceptable carrier selected on the basis of a chosen route of administration and standard pharmaceutical practice as described, for example, in Remington: The Science and Practice of Pharmacy (Gennaro, 21^{st} Ed. Mack Pub. Co., Easton, PA (2005)). Provided herein is a pharmaceutical composition comprising at least one conjugate described herein, or a stereoisomer, pharmaceutically acceptable salt, amide, ester, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

In one aspect, the disclosure provides a pharmaceutical composition comprising a herein described conjugate, or a pharmaceutically acceptable salt or solvate thereof, and a pharmaceutically acceptable excipient or carrier. In certain embodiments, the conjugate as described is substantially pure, in that it contains less than about 10%, less than about 5%, or less than about 1%, or less than about 0.1%, of other organic small molecules, such as unreacted intermediates or synthesis by-products that are created, for example, in one or more of the steps of a synthesis method.

Pharmaceutical compositions can include pharmaceutically acceptable carriers, diluents or excipients. Exemplary pharmaceutically acceptable carriers include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary components (e.g., preservatives, antibacterial, antiviral and antifungal agents) can also be incorporated into the compositions. Pharmaceutical compositions can be formulated to be compatible with a particular local or systemic route of administration. Thus, pharmaceutical compositions include carriers, diluents, or excipients suitable for administration by particular routes.

The conjugates and pharmaceutical compositions of the current disclosure can be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. The term parenteral as used herein includes e.g., subcutaneous, intravenous, intramuscular, intrasternal, intraperitoneal, and infusion techniques. The term parenteral also includes injections, into the eye or ocular, intravitreal, intrabuccal, transdermal, intranasal, into the brain, including intracranial and intradural, into the joints, including ankles, knees, hips, shoulders, elbows, wrists, and the like, and in suppository form. In certain embodiments, the conjugates and/or formulations are administered orally. In certain embodiments, the conjugates and/or formulations are administered by systemic administration. In certain embodiments, the conjugates and/or formulations are administered parenterally. In certain embodiments, the conjugates and/or formulations are administered locally at a targeted site.

In some embodiments, conjugates, or pharmaceutically acceptable salts or solvates thereof, and pharmaceutical compositions described herein are administered via parenteral injection as liquid solution, which can include other chemical components, such as carriers, stabilizers, diluents, dispersing agents, suspending agents, thickening agents, preservatives, or excipients. Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing or dispersing agents. Pharmaceutical formulations for parenteral administration include aqueous solutions of the active conjugates in water soluble form. Solutions or suspensions used for parenteral, intradermal, or subcutaneous application can include: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid, gentisic acid, or sodium bisulfite; chelating agents such as ethylenediaminetetraacetic acid; buffers such as acetates, citrates or phosphates; surfactants such as polysorbate 80; and agents for the adjustment of tonicity such as sodium chloride or dextrose. pH can be adjusted with acids or bases, such as hydrochloric acid or sodium hydroxide. In some embodiments, the pharmaceutical composition comprises a reductant. The presence of a reductant can help minimize potential radiolysis. In some embodiments, the reductant is ascorbic acid, gentisic acid, sodium thiosulfate, citric acid, tartaric acid, or a combination thereof.

Pharmaceutical compositions comprising the conjugates or pharmaceutically acceptable salts or solvates thereof described herein can be prepared according to standard techniques and further comprise a pharmaceutically acceptable carrier. In some embodiments, normal saline can be employed as the pharmaceutically acceptable carrier. Other suitable carriers include, e.g., water, buffered water, 0.9% isotonic saline, 0.4% saline, 0.3% glycine, and the like, including glycoproteins for enhanced stability, such as albumin, lipoprotein, globulin, etc. These compositions can be sterilized by conventional sterilization techniques. The resulting aqueous solutions may be packaged for use or filtered under aseptic conditions and lyophilized. In some embodiments, the lyophilized preparation is combined with a sterile aqueous solution prior to administration. The compositions can contain pharmaceutically acceptable auxiliary substances as appropriate to approximate physiological conditions, such as pH adjusting and buffering agents, tonicity adjusting agents and the like, for example, sodium acetate, sodium lactate, sodium chloride, potassium chloride, calcium chloride, sodium lactate, sorbitan monolaurate, triethanolamine oleate, etc. Pharmaceutical compositions can be selected according to their physical characteristic, including, but not limited to fluid volumes, viscosities and other parameters in accordance with the particular mode of administration selected. The amount of conjugates administered can depend upon the particular targeting moiety used, the disease state being treated, the therapeutic agent being delivered, and the judgment of the clinician.

The concentration of the conjugates or pharmaceutically acceptable salts or solvates thereof described herein in the pharmaceutical formulations can vary. In some embodiments, the conjugate is present in the pharmaceutical composition from about 0.05% to about 1% by weight, about 1% to about 2% by weight, about 2% to about 5% by weight, about 5% to about 10% by weight, about 10% to about 30% by weight, about 30% to about 50% by weight, about 50% to about 75% by weight, or about 75% to about 99% by weight.

Pharmaceutical compositions are administered in a manner appropriate to the disease to be treated. An appropriate dose and a suitable duration and frequency of administration will be determined by such factors as the condition of the subject, the type and severity of the subject's disease, the particular form of the active ingredient, and the method of administration. In some embodiments, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the subject.

The amount of conjugates or pharmaceutically acceptable salts or solvates thereof and/or pharmaceutical compositions administered can be sufficient to deliver a therapeutically effective dose of the particular subject. In some embodiments, conjugate dosages can be between about 0.1 μg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of the conjugate or a pharmaceutically acceptable salt or solvate thereof described herein for methods of treating a disease as described herein is about 0.001 mg/kg to about 1 mg/kg body weight of the subject per dose. In some embodiments, the dose of conjugate or a pharmaceutically acceptable salt or solvate thereof described herein for the described methods is about 0.001 mg to about 1000 mg per dose for the subject being treated. In some embodiments, a conjugate or a pharmaceutically acceptable salt or solvate thereof described herein is administered to a subject at a dosage of from about 0.01 mg to about 500 mg, from about 0.01 mg to about 100 mg, or from about 0.01 mg to about 50 mg. In some embodiments, a conjugate or a pharmaceutically acceptable salt or solvate thereof described herein is administered to a subject at a dosage of about 0.01 picomole to about 1 mole, or about 0.1 picomole to about 0.1 mole. In some embodiments, a conjugate or a pharmaceutically acceptable salt or solvate thereof described herein is administered to a subject at a dosage of about 0.01 Gbq to about 1000 Gbq, or about 0.5 Gbq to about 100 Gbq. In some embodiments, the dose is administered once a day, 1 to 3 times a week, 1 to 4 times a month, or 1 to 12 times a year.

The pharmaceutical formulations can be packaged in unit dosage form for ease of administration and uniformity of dosage. A unit dosage form can refer to physically discrete units suited as unitary dosages for the subject to be treated; each unit containing a predetermined quantity of active compound calculated to produce the desired therapeutic effect in association with the pharmaceutical carrier or excipient.

### IV. METHOD OF TREATMENT

In one aspect, the disclosure provides methods of treating a disease or condition in a subject in need thereof. In some embodiments, the disease or disorder is characterized by overexpression of EphA2 in diseased tissue. In some embodiments, the methods comprise administering a conjugate or a pharmaceutically acceptable salt or solvate thereof described herein, or a pharmaceutical composition comprising the same to the subject in need thereof. In some embodiments, provided herein is a method of providing a therapeutic and/or prophylactic benefit to a subject in need thereof comprising administering a compound or pharmaceutical composition described herein.

In some embodiments, the methods comprise administering to a subject a therapeutically effective amount of a conjugate or a pharmaceutically acceptable salt or solvate thereof. In some embodiments, the conjugate or pharmaceutically acceptable salt or solvate thereof is administered in a pharmaceutical composition. In some embodiments, the subject has cancer. In some embodiments, the cancer is a solid tumor or hematological cancer.

In one aspect, provided herein is a method of treating cancer by administering a herein described conjugate or a pharmaceutically acceptable salt or solvate thereof to a subject in need thereof. According to a further aspect of the disclosure, there is provided a drug conjugate as defined herein, for use in preventing, suppressing or treating a disease or disorder characterized by overexpression of EphA2 in diseased tissue (such as a tumor). In one embodiment, the EphA2 is mammalian EphA2. In a further embodiment, the mammalian EphA2 is human EphA2.

In one aspect, provided herein is a method of preventing, suppressing or treating a disease or disorder characterized by overexpression of EphA2 in diseased tissue (such as a tumour), which comprises administering to a patient in need thereof a conjugate described herein (e.g., including a peptide, a metal chelator and a radionuclide). In some embodiments, the disease or disorder characterized by overexpression of EphA2 in diseased tissue is a cancer.

Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, a subject or population of subjects to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a subject or population of subjects to be treated with a pharmaceutical composition of the present disclosure have a hematological cancer. In some embodiments, the subject has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a subject or population of subjects to be treated having the cancer selected from the group consisting of ovarian cancer, lung cancer and melanoma.

In some embodiments, provided herein are methods and compositions for treating a disease or condition. Exemplary disease or condition includes refractory or recurrent malignancies whose growth may be inhibited using the methods of treatment of the present disclosure. In some embodiments, the disease or condition is a cancer. In some embodiments, the cancer is breast cancer, head and neck squamous cell carcinoma, non-small cell lung cancer, hepatocellular cancer, bladder cancer, colorectal cancer, gastric adenocarcinoma, ovarian cancer, melanoma, or advanced cancer. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is pancreatic cancer. In some embodiments, a cancer to be treated by the methods of the present disclosure is non-small cell lung cancer, ovarian cancer, or bladder cancer. In some embodiments, a cancer to be treated by the methods of the present disclosure is non-small cell lung cancer. In some embodiments, a cancer to be treated by the methods of the present disclosure is bladder cancer. In some embodiments, a cancer to be treated by the methods of the present disclosure is ovarian cancer.

Further examples of cancers (and their benign counterparts) which may be treated include, but are not limited to tumors of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); hematological malignancies (i.e. leukemias, lymphomas) and premalignant hematological disorders and disorders of borderline malignancy including hematological malignancies and related conditions of lymphoid lineage (for example acute lymphocytic leukemia (ALL), chronic lymphocytic leukemia, B-cell lymphomas such as diffuse large B-cell lymphoma, follicular lymphoma, Burkitt's lymphoma, mantle cell lymphoma, T-cell lymphomas and leukemias, natural killer cell lymphomas, Hodgkin's lymphomas, hairy cell leukemia, monoclonal gammopathy of uncertain significance, plasmacytoma, multiple myeloma, and post-transplant lymphoproliferative disorders), and hematological malignancies and related conditions of myeloid lineage (for example acute myelogenous leukemia, chronic myelogenous leukemia, chronic myelomonocytic leukemia, hypereosinophilic syndrome, myeloproliferative disorders such as polycythemia vera, essential thrombocythemia and primary myelofibrosis, myeloproliferative syndrome, myelodysplastic syndrome, and promyelocytic leukemia); tumors of mesenchymal origin, for example sarcomas of soft tissue, bone or cartilage such as osteosarcomas, fibrosarcoma's, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumors, benign and malignant histiocytomas, and dermatofibrosarcoma protuberans; tumors of the central or peripheral nervous system (for example astrocytoma's, gliomas and glioblastomas, meningiomas, ependymomas, pineal tumors and schwannomas); endocrine tumors (for example pituitary tumors, adrenal tumors, islet cell tumors, parathyroid tumors, carcinoid tumors and medullary carcinoma of the thyroid); ocular and adnexal tumors (for example retinoblastoma); germ cell and trophoblastic tumors (for example teratomas, seminomas, dysgerminomas, hydatidiform moles and choriocarcinomas); and pediatric and embryonal tumors (for example medulloblastoma, neuroblastoma, Wilms tumor, and primitive neuroectodermal tumors); or syndromes, congenital or otherwise, which leave the patient susceptible to malignancy (for example Xeroderma Pigmentosum).

In some embodiments, the cancer is selected from glioblastoma, prostate cancer, lung cancer, breast cancer, gastric cancer, ovarian cancer, bladder cancer, colon cancer, esophageal cancer, multiple myeloma and fibrosarcoma. In some embodiments, the cancer is selected from: breast cancer, lung cancer, gastric cancer, pancreatic cancer, prostate cancer, liver cancer, glioblastoma and angiogenesis. In some embodiments, the cancer is selected from: prostate cancer, lung cancer (such as non-small cell lung carcinomas (NSCLC)), breast cancer (such as triple negative breast cancer), gastric cancer, ovarian cancer, esophageal cancer, multiple myeloma and fibrosarcoma. In some embodiments, the cancer is prostate cancer. In some embodiments, the conjugate is useful for preventing, suppressing or treating solid tumors such as fibrosarcoma's and breast, and non-small cell lung carcinomas. In some embodiments, the cancer is selected from lung cancer, such as non-small cell lung carcinomas (NSCLC). In some embodiments, the cancer is breast cancer. In some embodiments, the breast cancer is triple negative breast cancer. In some embodiments, the breast cancer is Herceptin resistant breast cancer. In some embodiments, the subject has failed to respond to Herceptin. In some embodiments, the cancer is gastric cancer. In some embodiments, the cancer is ovarian cancer. In some embodiments, the cancer is esophageal cancer. In some embodiments, the cancer is multiple myeloma. In some embodiments, the cancer is fibrosarcoma.

In some embodiments, provided herein are methods for killing a cell comprising contacting the cell with a conjugate or a pharmaceutically acceptable salt or solvate thereof. In some embodiments, the cell expresses EphA2. In some embodiments, the cell over-expresses EphA2. In some embodiments, the conjugate or pharmaceutically acceptable salt or solvate thereof binds to a structure on the cell, wherein the structure is an EphA2. In some embodiments, the conjugate or pharmaceutically acceptable salt or solvate thereof releases a number of alpha particles by natural radioactive decay. In some embodiments, the conjugate or pharmaceutically acceptable salt or solvate thereof releases a number of beta particles, gamma rays, and/or Auger electrons by natural radioactive decay. The conjugate described herein can kill a cell by radiation. In some embodiments, the conjugate kills the cell directly by radiation. In some embodiments, the radiation creates, in the cell, oxidized bases, abasic sites, single-stranded breaks, double-stranded breaks, DNA crosslink, chromosomal rearrangement, or a combination thereof. In some embodiments, the conjugate kills the cell by inducing double-stranded DNA breaks. In some embodiments, the released alpha particles are sufficient to kill the cell. In some embodiments, the released alpha particles are sufficient to stop cell growth. In some embodiments, the conjugate kills the cell indirectly via the production of reactive oxygen species (ROS) such as free hydroxyl radicals. In some embodiments, the conjugate kills the cell indirectly by releasing tumor antigens from one or more different cells, which can have vaccine effect. In some embodiments, the conjugate kills the cell by abscopal effect. In some embodiments, the cell is a cancer cell. In some embodiments, the method comprises killing a cell with an alpha-particle emitting radionuclide.

After contacting a cell, the described conjugate can be internalized by the cell. The internalization can be mediated by cell receptors, cell membrane endocytosis, etc. In some embodiments, the described conjugate is internalized by a cell through EphA2. In some embodiments, rapid internalization rate into cancer cells accompanied by a slow externalization rate can offer therapeutic benefit.

In one aspect, the disclosed conjugate or a pharmaceutically acceptable salt or solvate thereof is configured to treat cancer by ablating tumor cells. In some embodiments, the conjugate or a pharmaceutically acceptable salt or solvate thereof does not modulate the biology of the tumor cell and/or the surrounding stroma. In some embodiments, the conjugate or a pharmaceutically acceptable salt or solvate thereof does not modulate immune cells. In some embodiments, the ablating of tumor cells can lead to a downstream immunological cascade.

In addition to the methods of treatment described above, the conjugates and compositions described herein can be used to image, and/or as part of a treatment for diseases. Conjugates for imaging applications, e.g., single-photon emission computed tomography (SPECT) and positron emission tomography (PET), can comprise a radionuclide suitable for use as imaging isotopes such as the isotopes in Table 7 labeled “Dx”. Accordingly, the conjugate can be administered as a companion diagnostic.

In one aspect, described herein is a method of treatments that comprises administering a first conjugate and a second conjugate. The first conjugate can be used as companion diagnostics and the second conjugate can be used for therapeutics. In some embodiments, the first conjugate and the second conjugate have the same structure except for the radionuclide. In some embodiments, the first conjugate comprises a gamma particle emitting radionuclide. In some embodiments, the first conjugate comprises a radionuclide of Table 7 labeled “Dx”. In some embodiments, the first conjugate comprises a radionuclide selected from Lu-177, In-111, Ga-68, Cu-64, and Zr-89. In some embodiments, the first conjugate comprises a covalent radionuclide selected from ¹⁸F, ⁷⁴As, ⁷⁶Br, ¹²³I, ¹²⁴I, and ¹²⁵I. In some embodiments, the second conjugate comprises an alpha or beta-particle emitting radionuclide. In some embodiments, the second conjugate comprises a radionuclide of Table 7 labeled “Tx”. In some embodiments, the second conjugate comprises Ac-225. In some embodiments, the second conjugate comprises a covalent radionuclide selected from ¹³¹I and ²¹¹At. In some embodiments, the method comprises administering (i) a first conjugate comprising a radionuclide configured for companion diagnostic (such as PET imaging) and (ii) a second conjugate comprising a radionuclide selected from an alpha or beta-particle emitter, wherein the first and the second conjugate have the same structure except for the radionuclide.

In one aspect, described herein is a method of diagnosing or imaging a cancer in a subject in need thereof, comprising administering to the subject a conjugate or a pharmaceutical composition described herein.

In some embodiments, the subject is 1 to 100 years old. In some embodiments, the subject is 5 to 10, 5 to 15, 5 to 18, 5 to 25, 5 to 35, 5 to 45, 5 to 55, 5 to 65, 5 to 75, 10 to 15, 10 to 18, 10 to 25, 10 to 35, 10 to 45, 10 to 55, 10 to 65, 10 to 75, 15 to 18, 15 to 25, 15 to 35, 15 to 45, 15 to 55, 15 to 65, 15 to 75, 18 to 25, 18 to 35, 18 to 45, 18 to 55, 18 to 65, 18 to 75, 25 to 35, 25 to 45, 25 to 55, 25 to 65, 25 to 75, 35 to 45, 35 to 55, 35 to 65, 35 to 75, 45 to 55, 45 to 65, 45 to 75, 55 to 65, 55 to 75, or 65 to 75 years old. In some embodiments, the subject is at least 5, 10, 15, 18, 25, 35, 45, 55, or 65 years old. In some embodiments, the subject is at most 10, 15, 18, 25, 35, 45, 55, 65, or 75 years old.

### Combination Therapy

In some embodiments, a conjugate described herein can be administered alone or in combination with one or more additional therapeutic agents. For example, the combination therapy can include a composition comprising a conjugate described herein co-formulated with, and/or co-administered with, one or more additional therapeutic agents, e.g., one or more anti-cancer agents, e.g., cytotoxic or cytostatic agents, immune checkpoint inhibitors, hormone treatment, vaccines, and/or immunotherapies. In some embodiments, the conjugate is administered in combination with other therapeutic treatment modalities, including surgery, cryosurgery, and/or chemotherapy. Such combination therapies may advantageously utilize lower dosages of the administered therapeutic agents, thus avoiding possible toxicities or complications associated with the various monotherapies.

When administered in combination, two (or more) different treatments can be delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In some embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the herein-described conjugate is used in combination with a chemotherapeutic agent, e.g., a DNA damaging chemotherapeutic agent, a platinum based agent, a topoisomerase inhibitor, a taxane, an antimetabolite, a vinca alkaloid, or an anthracycline. In some embodiments, the herein-described conjugate is used in combination with a radiation sensitizer, which makes tumor cells more sensitive to radiation therapy. In some embodiments, the herein-described conjugate is used in combination with a DNA damage repair inhibitor (or DNA damage response (DDR) inhibitor). In some embodiments, the DNA damage repair inhibitor or DDR inhibitor is a poly (ADP-ribose) polymerase (PARP) inhibitor. In some embodiments, the herein-described conjugate is used in combination with an immune checkpoint inhibitor. In some embodiments, the immune checkpoint inhibitor is a PD-L1 inhibitor, a PD-1 inhibitor, or a CTLA-4 inhibitor. In some embodiments, the herein-described conjugate is used in combination with a chemotherapeutic agent, a PARP inhibitor, and/or an immune checkpoint inhibitor. In some embodiments, the herein-described conjugate is used in combination with a chemotherapeutic agent, a PARP inhibitor, and an immune checkpoint inhibitor.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### A: Synthesis of the Conjugates

Unless otherwise stated in the present specification, the following abbreviations are used according to the following meanings:
Alloc allyloxycarbonyl
aq. aqueous
Biotin-OSu Biotin N-hydroxysuccinimide ester (CAS 35013-72-0)
Boc tert-butyloxycarbonyl
CIAcOH chloroacetic acid
ClAcOSu N-succinimidyl 2-chloroacetate (CAS 27243-15-8)
DCM dichloromethane (CAS 75-09-2)
DIC N,N′-diisopropylcarbodiimide (CAS 693-13-0)
DIPEA, DIEA N,N-diisopropylethylamine (CAS 7087-68-5)
DMF N,N-dimethylformamide (CAS 68-12-2)
DODT 2,2′-(ethylenedioxy) diethanethiol (CAS 14970-87-7)
EDCI-HCl N-(3-dimethylaminopropyl)-N′-ethylcarbodiimide hydrochloride (CAS 25952-53-8)
eq equivalent
Et ethyl
Et3N, TEA triethylamine (CAS 121-44-8)
Fmoc 9-fluorenylmethoxycarbonyl
hr hour
HATU 1-[bis(dimethylamino)methylene]-1H-1,2,3-triazolo[4,5-b]pyridinium 3-oxide hexafluorophosphate (CAS 148893-10-1)
HOSu N-hydroxysuccinimide (CAS 6066-82-6)
iPrOH/IPA isopropanol
M molar
min minutes
NHS N-hydroxysuccinimide (CAS 6066-82-6)
NMP N-methylpyrrolidone (CAS 872-50-4)
Pd(PPh3)4 tetrakis(triphenylphosphine) palladium (0) (CAS: 14221-01-3)
Ph phenyl
rpm rotations per minute
rt room temperature
SPPS solid phase peptide synthesis
Su succinimidyl
SulfoCy5 sulfo Cyanine5
tert tertiary
TFA trifluoroacetic acid (CAS 76-05-1)
TIS triisopropylsilane (CAS 6485-79-6)
TR retention time
Trt trityl.

### Example A1. Analytical Conditions

Solid phase peptide synthesis (SPPS) was performed in a standard manual reaction vessel under nitrogen. Rink Amide-MBHA resin was purchased from Sunresin New Materials Co. (China). Fmoc protected amino acids were purchased from GL Biochem (China). HBTU and HATU were purchased from Highfine Biotech Co. (China). Piperidine was purchased from Damao Chemical Reagent Factory (China). The peptides and their derivatives were purified on a Gilson GX-281 preparative HPLC system using reverse-phase C18 columns (Gemini, 5 μm, 110 Å+luna, 10 μm, 100 Å) at 30 °C. HPLC solvents consisted of H₂O containing 0.075% trifluoroacetic acid (mobile phase A) and CH₃CN (mobile phase B).

High performance liquid chromatography (HPLC) analyses were performed on an Agilent 1260 series equipped with a binary pump G7112A, micro vacuum degasser, standard autosampler ALS G7129A, thermostated column compartment TCC G7116A, variable wavelength detector VWD G7114A, and data were analyzed by OpenLab CDS 2.2 network workstation software from Agilent Technologies. HPLC solvents consisted of H₂O containing 0.1% trifluoroacetic acid (mobile phase A) and CH₃CN containing 0.075% trifluoroacetic acid (mobile phase B). Conditions: a Phenomenex Gemini-NX C-18 (5 μm, 110 Å, 4.6×150 mm) column was used with a flow rate of 1.0 mL/min.

LC-MS analyses were carried out on an Agilent 1200 series coupled to an Agilent MSD G6125C, equipped with a binary pump G7112A, micro vacuum degasser, standard autosampler ALS G7129A, thermostated column compartment TCC G7116A, variable wavelength detector VWD G7114A, and data were analyzed by OpenLab CDS 2.3 standalone workstation software from Agilent Technologies. HPLC solvents consisted of H₂O containing 0.1% trifluoroacetic acid (mobile phase A) and CH₃CN containing 0.075% trifluoroacetic acid (mobile phase B). Conditions: a Waters Xbridge C18 (3.5 μm, 0.1×30 mm) column was used with a flow rate of 1.2 mL/min, with UV detection at 220 nm.

### Example A2: Synthesis of Peptidyl-Resin 1

To the swollen Fmoc-MBHA Resin (0.3 mmol, 0.331 mmol/g, 1.00 equiv) was removed via 20 min agitation with 20% piperidine in DMF followed by filtration and washing. Then the resin was added Fmoc-D-Lys (Boc)-OH (0.282 g, 0.6 mmol), HBTU (0.228 g, 0.6 mmol) and DIEA (0.3 mL, 1.8 mmol) in dry DMF. The mixture was agitated for 30 min under nitrogen. After the reaction solution was removed through filtration, the resin was washed three times with DMF (30 mL). The Fmoc protecting group was removed via two times (5 min and 10 min) agitation with 20% piperidine in DMF followed by filtration and washing.

Subsequent amino acids were coupled using Fmoc-protected amino acid (2.0 equiv), HBTU (2.0 equiv) and DIEA (6.0 equiv) in dry DMF, shaking for 30 min. Pre-activation of any amino acid was not performed prior to coupling. Between amino acid couplings, the Fmoc protecting group was removed via two times (5 min and 10 min) agitation with 20% piperidine in DMF followed by filtration and washing. Success of Fmoc removal steps and amino acid couplings were monitored qualitatively using a ninhydrin test. (See the Table-A2-1).

TABLE A2-1
Procedure of SPPS of linear peptide
sequence: ClAc-da-Me3Py-N-L-Hgn-MeE-W1Me-KCOpipzaa-W1Me-T-HomoArg-C-Dlys-NH2 (SEQ ID NO: 146)

| Resin | | 30 | | | | | | | | | |
|---|---|---|---|---|---|---|---|---|---|---|---|
| Swell | DCM | min | resin | MBHA | loading | 0.331 mmol/g | | | | | |
| Reaction (DMF) | | | Coupling | | | | | Deprotection | | | |
| | Amino | | Coupling | | reaction | detection | testing | | reaction | detection | testing |
| # | Acid | equiv | reagent | equiv | time | reagent | result | reagent | time | reagent | result |
| 1 | Fmoc-D- | 2.0 | HBTU/ | 2.0/ | 1 hr | ninhydrin | − | 20% | 5 min + | ninhydrin | + |
| | Lys(Boc)- | | DIEA | 4.0 | | | | piperidine/ | 10 min | | |
| | OH | | | | | | | DMF | | | |
| 2 | Fmoc-Cys | 2.0 | HBTU/ | 2.0/ | 1 hr | ninhydrin | − | 20% | 5 min + | ninhydrin | + |
| | (Trt)-OH | | DIEA | 4.0 | | | | piperidine/ | 10 min | | |
| | | | | | | | | DMF | | | |
| 3 | Fmoc- | 2.0 | HBTU/ | 2.0/ | 1 hr | ninhydrin | − | 20% | 5 min + | ninhydrin | + |
| | HomoArg(Pbf)- | | DIEA | 4.0 | | | | piperidine/ | 10 min | | |
| | OH | | | | | | | DMF | | | |
| 4 | Fmoc-Thr | 2.0 | HBTU/ | 2.0/ | 1 hr | ninhydrin | − | 20% | 5 min + | ninhydrin | + |
| | (tBu)-OH | | DIEA | 4.0 | | | | piperidine/ | 10 min | | |
| | | | | | | | | DMF | | | |
| 5 | Fmoc-Trp | 2.0 | HBTU/ | 2.0/ | 1 hr | ninhydrin | − | 20% | 5 min + | ninhydrin | + |
| | (1-Me)- | | DIEA | 4.0 | | | | piperidine/ | 10 min | | |
| | OH | | | | | | | DMF | | | |
| 6 | Dde-Lys | 2.0 | HBTU/ | 2.0/ | 1 hr | ninhydrin | − | 20% | 5 min + | ninhydrin | + |
| | (Fmoc)- | | DIEA | 4.0 | | | | piperidine/ | 10 min | | |
| | OH | | | | | | | DMF | | | |
| 7 | Bis(4- | 2.0 | DIEA | 4.0 | 1 hr | ninhydrin | − | | | | |
| | nitrophenyl) | | | | | | | | | | |
| | Carbonate | | | | | | | | | | |
| 8 | tert-butyl | 2.0 | DIEA | 4.0 | 1 hr | | | 5% NH2 | 20 min + | ninhydrin | + |
| | 1- | | | | | | | NH2/D | 20 min | | |
| | piperazinyl | | | | | | | MF | | | |
| | acetate | | | | | | | | | | |
| 9 | Fmoc-Trp | 2.0 | HBTU/ | 2.0/ | 1 hr | ninhydrin | − | 20% | 5 min + | ninhydrin | + |
| | (1-Me)- | | DIEA | 4.0 | | | | piperidine/ | 10 min | | |
| | OH | | | | | | | DMF | | | |
| 10 | Fmoc-N- | 2.0 | HBTU/ | 2.0/ | 1 hr | ninhydrin | − | 20% | 5 min + | chloranil | + |
| | Me-Glu | | DIEA | 4.0 | | | | piperidine/ | 10 min | | |
| | (OtBu)- | | | | | | | DMF | | | |
| | OH | | | | | | | | | | |
| 11 | Fmoc-Hgn | 2.0 | PyAop/ | 2.0/ | 2 hr | chloranil | − | 20% | 5 min + | ninhydrin | + |
| | (Trt)-OH | | DIEA | 4.0 | | | | piperidine/ | 10 min | | |
| | | | | | | | | DMF | | | |
| 12 | Fmoc-Dap | 2.0 | HBTU/ | 2.0/ | 1 hr | ninhydrin | − | 20% | 5 min + | ninhydrin | + |
| | (Boc)-OH | | DIEA | 4.0 | | | | piperidine/ | 10 min | | |
| | | | | | | | | DMF | | | |
| 13 | Fmoc-Gln | 2.0 | HBTU/ | 2.0/ | 1 hr | ninhydrin | − | 20% | 5 min + | ninhydrin | + |
| | (Trt)-OH | | DIEA | 4.0 | | | | piperidine/ | 10 min | | |
| | | | | | | | | DMF | | | |
| 14 | Fmoc- | 2.0 | HBTU/ | 2.0/ | 1 hr | ninhydrin | − | 20% | 5 min + | chloranil | + |
| | Me3Py- | | DIEA | 4.0 | | | | piperidine/ | 10 min | | |
| | OH | | | | | | | DMF | | | |
| 15 | Fmoc-D- | 2.0 | PyAop/ | 2.0/ | 2 hr | chloranil | − | 20% | 5 min + | ninhydrin | + |
| | Ala-OH | | DIEA | 4.0 | | | | piperidine/ | 10 min | | |
| | | | | | | | | DMF | | | |
| 16 | Cl-Ac | 2.0 | HOBT/ | 2.0/ | 3 hr | ninhydrin | − | | | | |
| | | | DIC | 4.0 | | | | | | | |

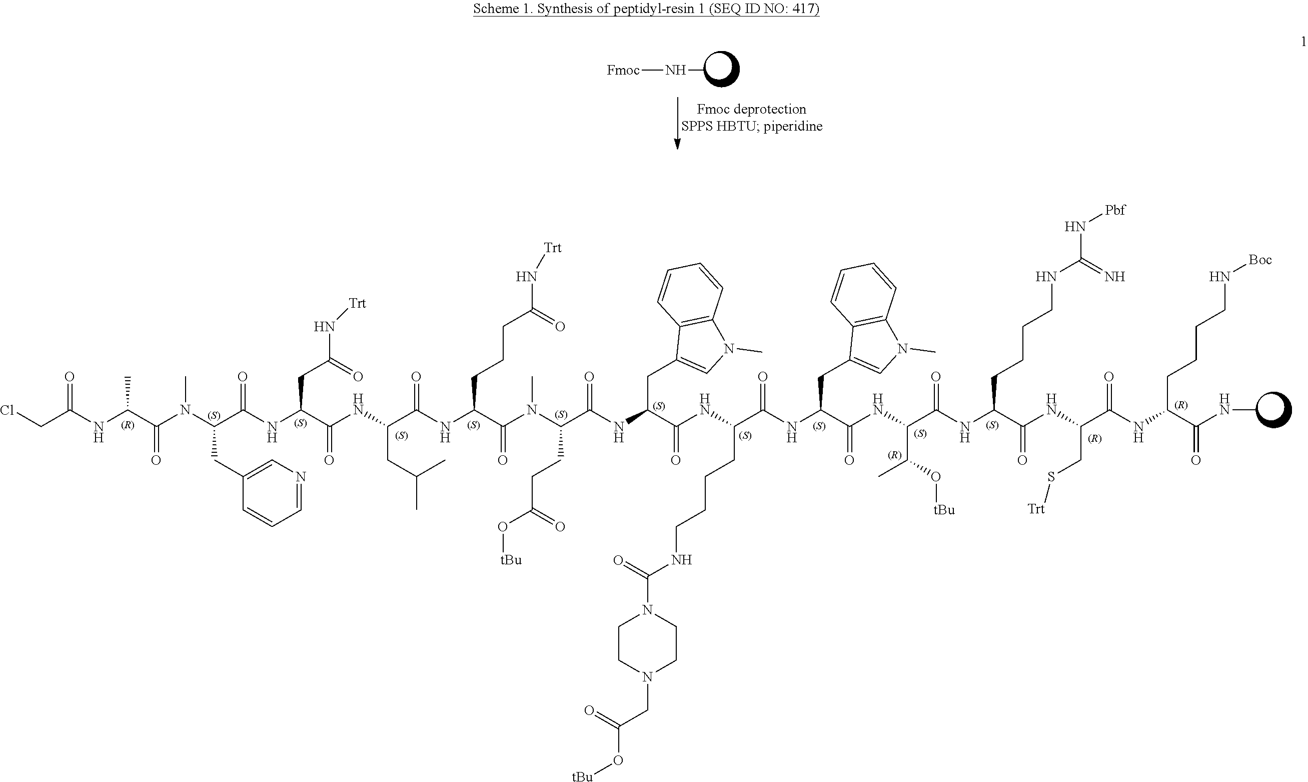

### Example A3: Synthesis of PDC_EphA2-00008010-C302

After the resin was washed three times with MeOH and dried under vacuum, a cocktail of trifluoroacetic acid/H2O/triisopropylsilane (95:2.5:2.5) was added. The resulting mixture was stirred for 2 h at room temperature, resin was filtered. To the collected TFA mixture solution, cold MTBE was added. The precipitated crude linear peptide-2 was collected through filtration and dried under vacuum.

Cyclization of peptide-2: To a solution of crude Peptide-2 (500 mg) in water (250 mL) and MeCN (250 mL) were added Et₃N while PH was adjusted to 8.0. The resulting mixture was stirred at room temperature for 1 hour. The mixture was purified by preparative HPLC to afford Peptide-3 (PDC_EphA2-00008010-C002) (130 mg, 26% yield, >90%) as a white solid.

To a solution of Peptide-3 (PDC_EphA2-00008010-C002) (70 mg) in DMF (5 mL) were added DOTA-OSu (2.0 eq), DIEA (3.0 eq). The resulting mixture was stirred at room temperature for 1 hour, the crude was purified by preparative HPLC to afford PDC_EphA2-00008010-C202 (43.0 mg, 62.8% yield, 97.53% purity) as a white solid.

Lu³⁺ complexation: To a solution of PDC_EphA2-00008010-C202 (33.0 mg, 13.8 μmol) in H₂O (5.0 mL) and MeCN (1.0 mL) was added LuCl₃ (5.2 mg, 18.0 μmol) and Na₂CO₃ (0.146 mg, 1.38 μmol). The resulting mixture was stirred at 70 °C for 1 h. The crude product was purified by preparative HPLC to afford PDC_EphA2-00008010-C302 (20.3 mg, 61.5% yield, 99.43% purity) as a white solid.

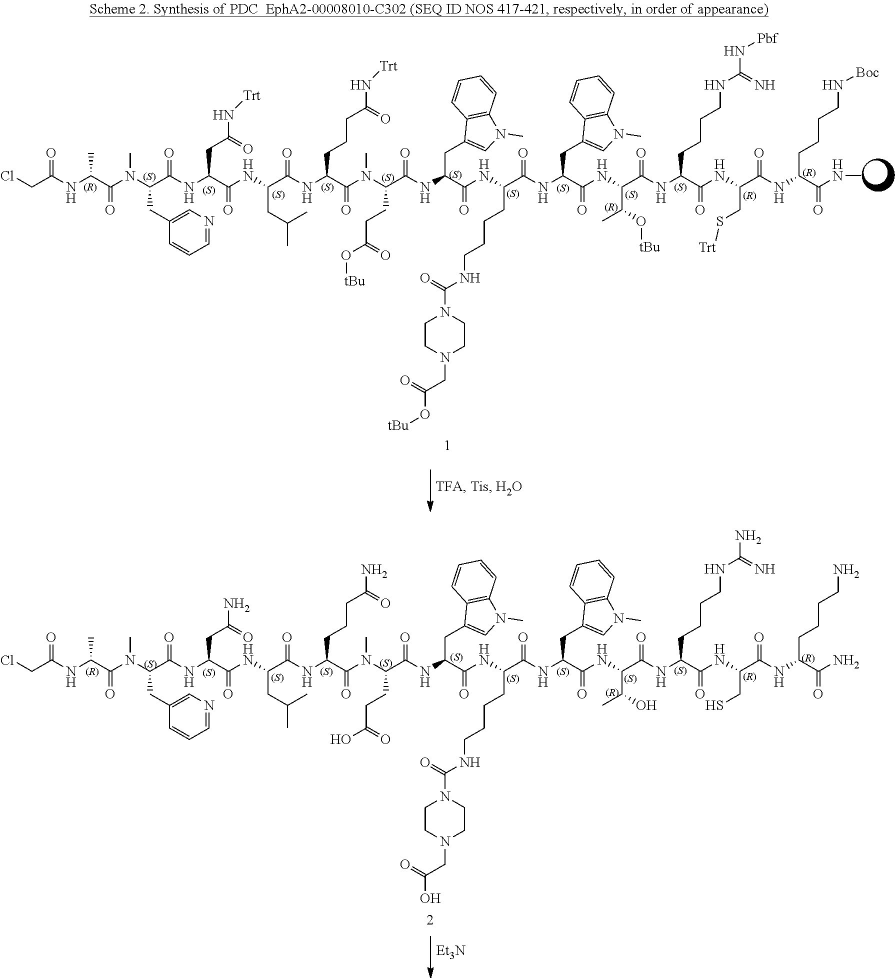

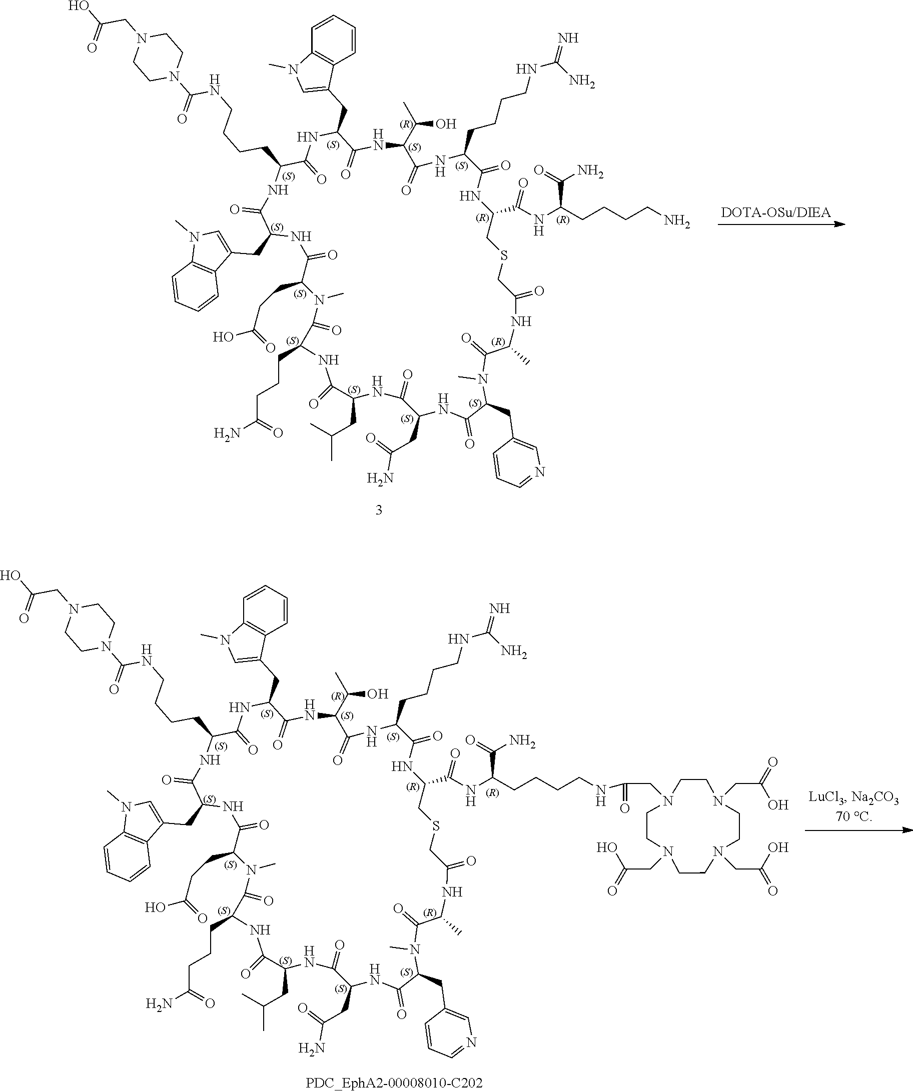

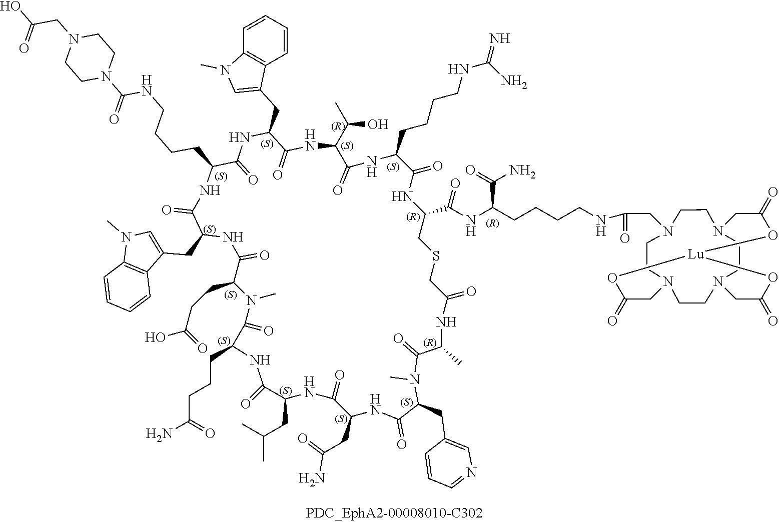

### Example A4: Synthesis of PDC_EphA2-00007196-C312

PDC_EphA2-00007196-C312 was synthesized according to the schemes below (SEQ ID NOS 200, 418 and 293, respectively, in order of appearance).

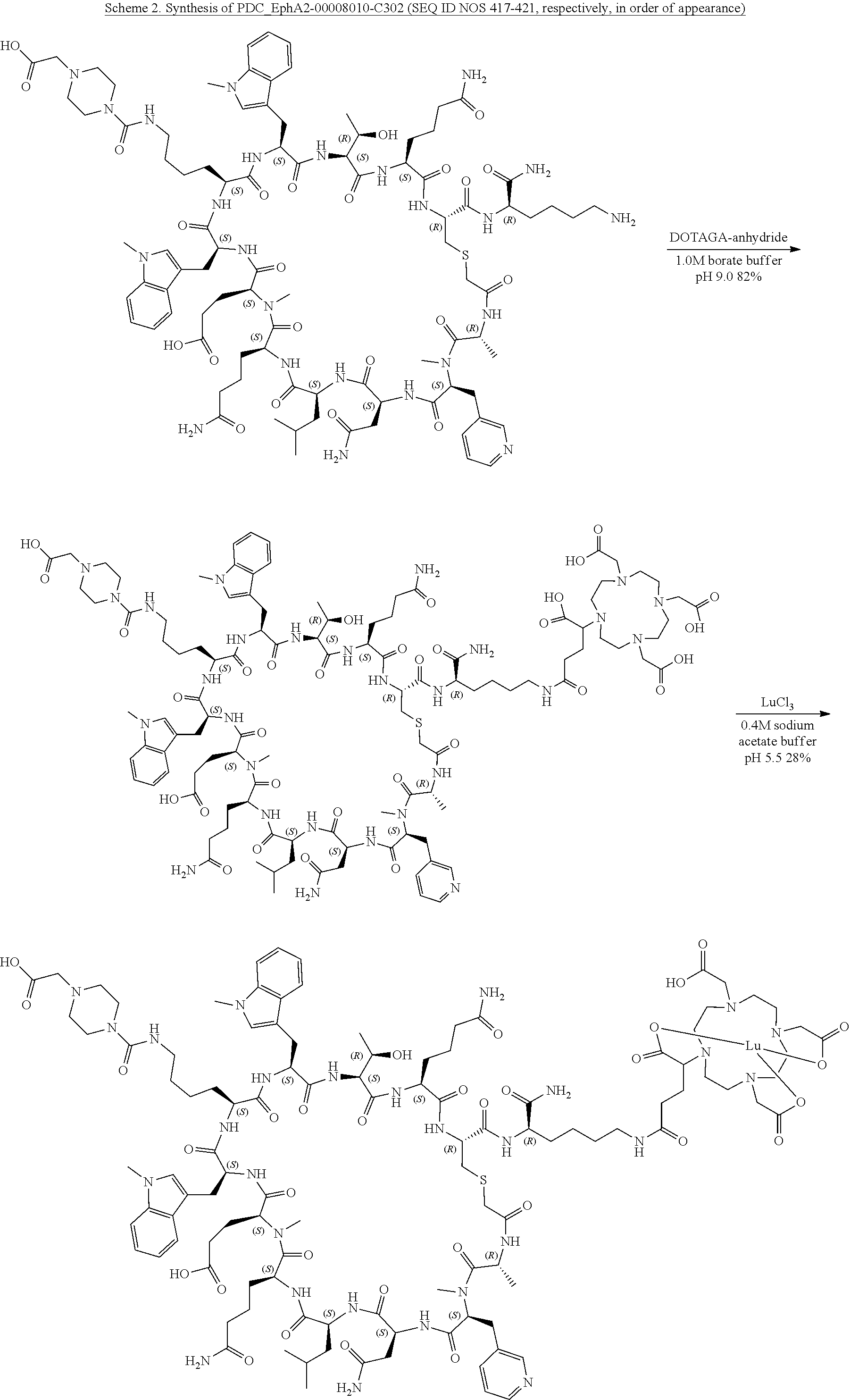

### Step 1 (SEQ ID NO 418).

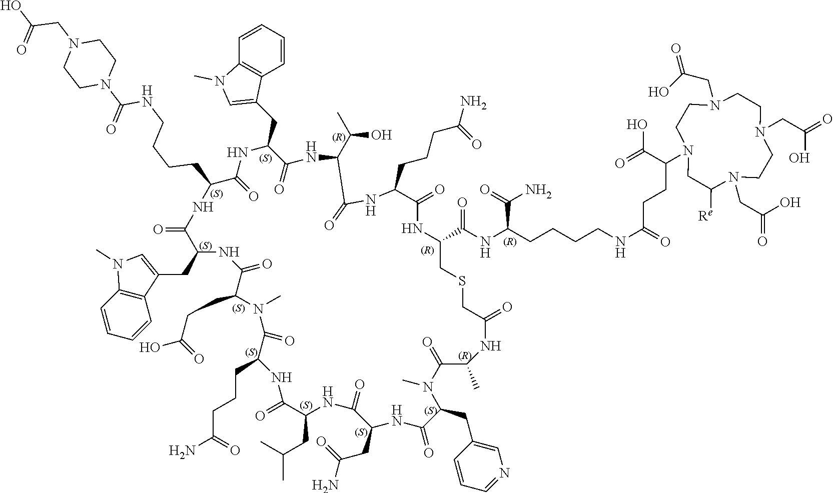

The starting material peptide-PDC_EphA2-00007196-C002 (11 mg, 4.99 μmol) was dissolved in 1.5 mL of 20× (1.0 M) borate buffer at pH 9.0, and the DOTAGA anhydride was added as a solid (4.44 mg, 2 eqmol, 9.98 μmol). After 3 h some starting material was still present (LC-MS monitoring), and additional 2 eqmol of chelator were added to enable complete conversion. The reaction was injected directly onto a prep HPLC column for purification (0 to 50% acetonitrile and water, using 0.1% TFA as modifier). Yield 10 mg, 82%. Ions found by LCMS: [M+H]⁺=2434.2, [(M+2H)/2]+=1217.6, [(M+3H)/3]+=812.1.

### Step 2 (SEQ ID NO: 293).

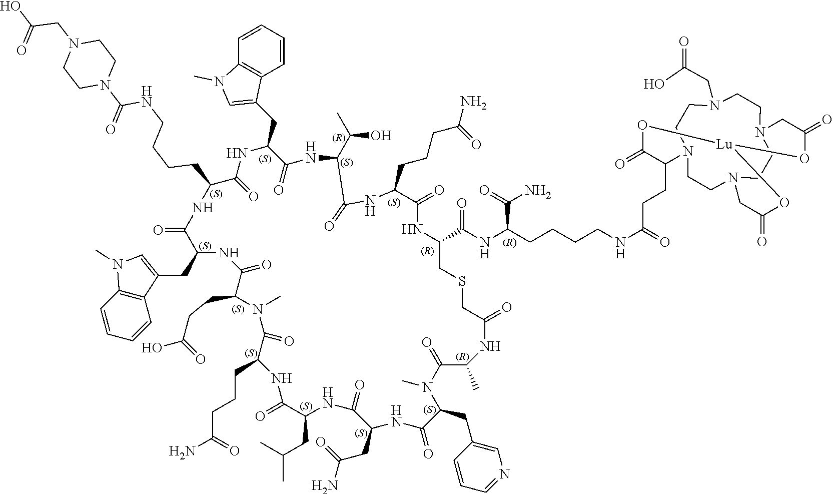

The product from step 1 (6 mg, 2.46 μmol) was dissolved in 500 μL of 0.4 M pH 5.5 sodium acetate buffer, and a water solution of Lutetium trichloride (104 μL of a 10 mg/mL solution, 3.7 μmol, 1.5 eqmol) was added to the solution. Upon full complexation (LC-MS monitoring), the reaction was injected directly onto a prep HPLC column for purification (0 to 50% acetonitrile and water, using 0.1% TFA as modifier). Yield 1.8 mg, 28%. Ions found by LCMS: [M+H]⁺=2609.1, [(M+2H)/2]+=1304.6, [(M+3H)/3]+=869.7.

### Example A5: Synthesis of PDC_EphA2-00007196-C322 (SEQ ID NO: 296)

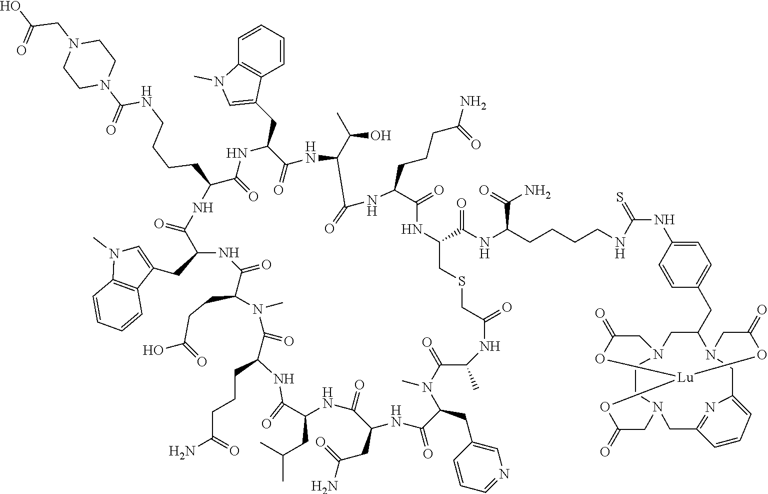

The title compound was synthesized in a similar manner to Example A4. Ions found by LCMS: [M+H]+=2678.1, [(M+2H)/2]+=1339.1, [(M+3H)/3]+=892.7, [(M+4H)/4]+=669.5. Example A6: Synthesis of PDC_EphA2-00007196-C332

The title compound was synthesized according to the schemes below (SEQ ID NOS 200, 419, 295, respectively, in order of appearance).

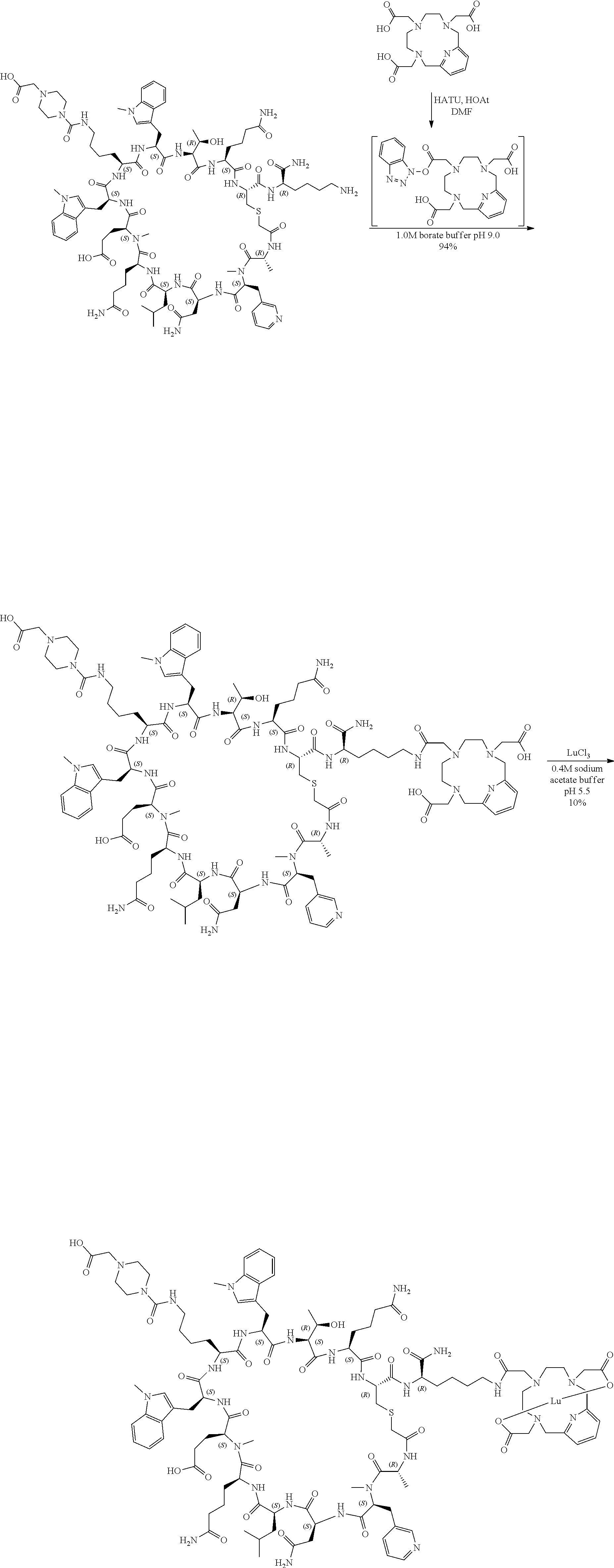

### Step 1 (SEQ ID NO: 419).

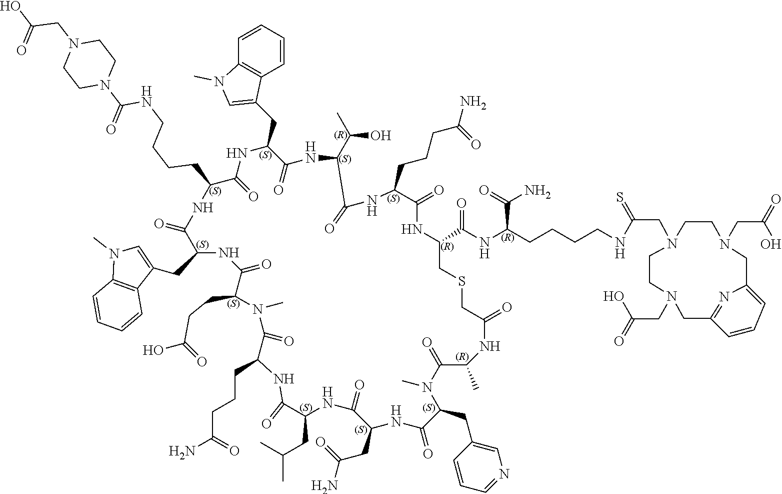

To a solution of PCTA (10.8 mg, 28.5 μmol) in DMF (85 μL) it was added 0.6 M HOAt in DMF (42.5 μL, 25 μmol), HATU (9.5 mg, 25 μmol) and finally DIPEA (65.3 μL, 375 μmol). This reaction was mixed efficiently, and the resulting solution was allowed to react for 10 minutes. The final total volume of this mixture was approximated to 200 μL. In a separate vessel, the starting material peptide-PDC_EphA2-00007196-C002 (11 mg, 4.99 μmol) was dissolved in 1.5 mL of 20× (1.0 M) borate buffer at pH 9.0, and half of the above freshly prepared solution was added, while the remaining half was added after 1 h. Full conversion was observed by LC-MS, and the reaction was injected directly onto a prep HPLC column for purification (0 to 50% acetonitrile and water, using 0.1% TFA as modifier). Yield 11 mg, 94%. Ions found by LCMS: [M+H]⁺=2338.1, [(M+2H)/2]+=1169.6, [(M+3H)/3]+=780.0.

### Step 2 (SEQ ID NO: 295)

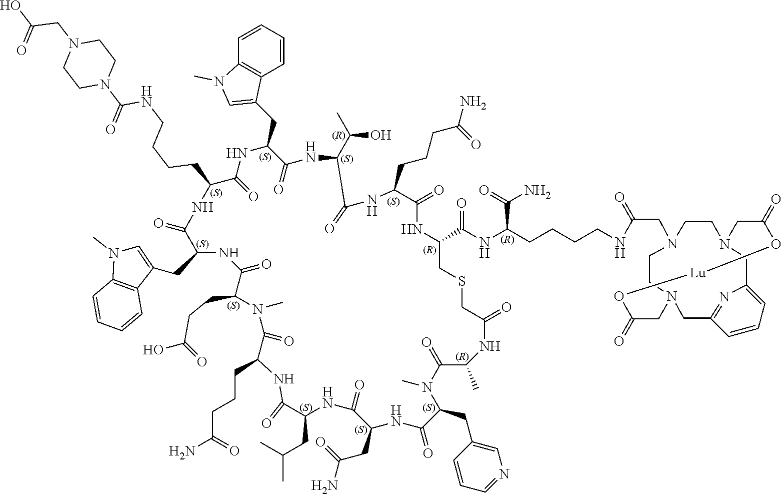

The product from step 1 (6 mg, 2.56 μmol) was dissolved in 500 μL of 0.4 M pH 5.5 sodium acetate buffer, and a water solution of Lutetium trichloride (216 μL of a 10 mg/mL solution, 7.68 μmol, 3.0 eqmol) was added to the solution. Upon full complexation (LC-MS monitoring), the reaction was injected directly onto a prep HPLC column for purification (0 to 50% acetonitrile and water, using 0.1% TFA as modifier). Yield 0.6 mg, 10%. Ions found by LCMS: [M+H]⁺=2513.1, [(M+2H)/2]+=1256.5, [(M+3H)/3]+=837.7.

### Example A7: Synthesis of PDC_EphA2-00007196-C342 (SEQ ID NO: 294)

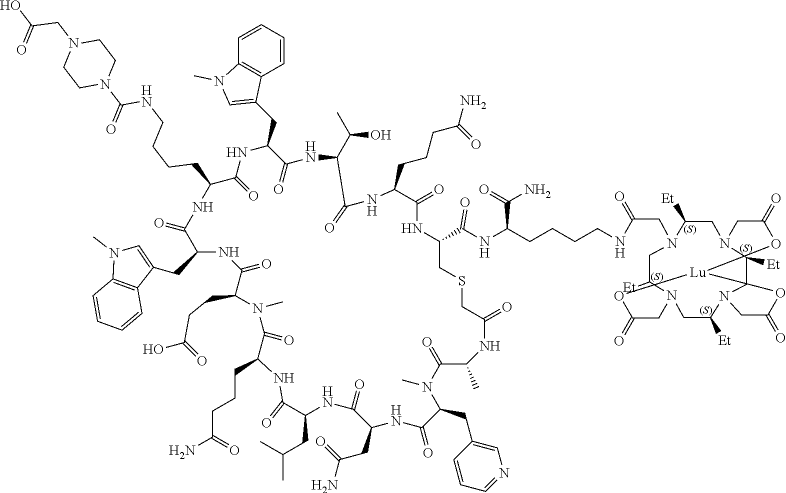

The target compound was synthesized in a similar manner to Example A6. The 2(S),2′(S),2″(S),2′(S)-tetra-Et-DOTA substrate was synthesized according to reference Nat Commun 9, 857 (2018). Ions found by LCMS: [M+H]⁺=2474.3, [(M+2H)/2]+=1324.6, [(M+3H)/3]+=883.1.

### Example A8: Synthesis of PDC_EphA2-00007196-C305

The title compound was synthesized according to the schemes below (SEQ ID NO: 200, 420, 297 and 297, respectively, in order of appearance).

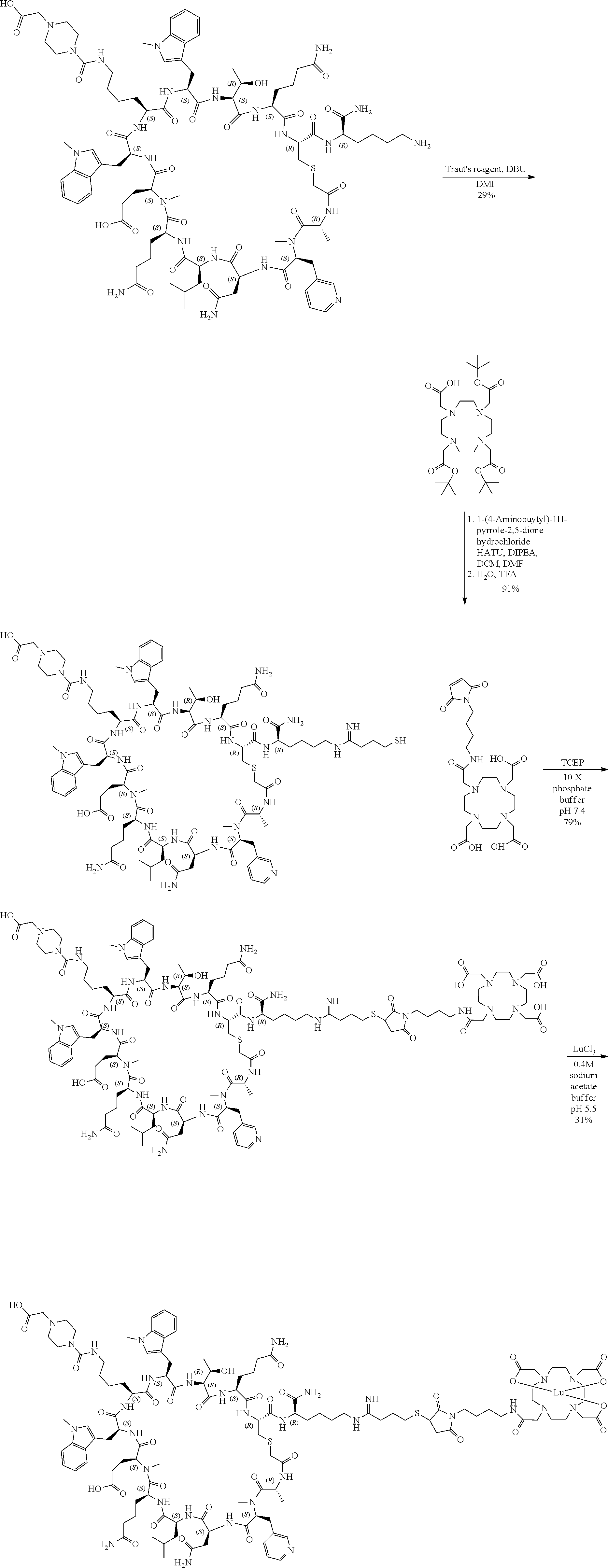

### Step 1 (SEQ ID NO: 420)

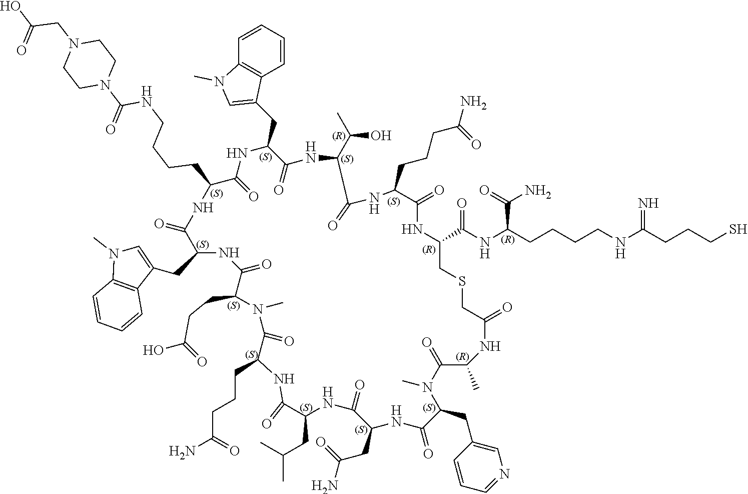

To a stirring solution of the starting material peptide-PDC_EphA2-00007196-C002 (11 mg, 4.99 umol) in DMF (1 mL) it was added DBU (14.9 μL, 99.8 μmol) and last Traut's reagent (7.6 mg, 54.9 umol). The reaction was stirred overnight, and then it was injected directly onto a prep HPLC column for purification (0 to 50% acetonitrile and water, using 0.1% TFA as modifier). Yield 3 mg, 29%. Ions found by LCMS: [M+H]⁺=2077.0, [(M+2H)/2]⁺=1039.0, [(M+3H)/3]⁺=693.0.

### Step 2

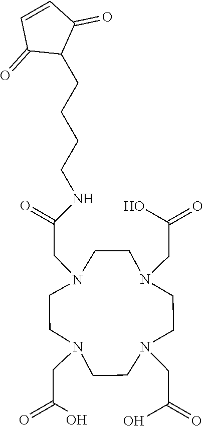 [SMILES: O=C(O)CN1CCN(CC(=O)O)CCN(CC(=O)NCCCCC2C(=O)C=CC2=O)CCN(CC(=O)O)CC1]

To a stirring solution of DOTA tri (tert-butyl) ester (40 mg, 69.8 μmol) in DMF (1.0 mL) it was added 1-(4-Aminobutyl)-1H-pyrrole-2,5-dione hydrochloride (15 mg, 73.3 μmol), HATU (29.2 mg, 76.8 umol) and last DIPEA (36.5 μL, 210 μmol). Upon completion (LC-MS monitoring), the reaction was injected directly onto a prep HPLC column for purification (5 to 100% acetonitrile and water, using 0.1% TFA as modifier). Evaporation of the relevant fractions afforded the desired intermediate. Yield 46 mg, 91%. Ions found by LCMS: [M+H]+=723.4, [M+H+Na]+=755.4. This material was treated with 4 mL of TFA:water=95:5. Upon completion (LC-MS monitoring), all the volatiles were evaporated and the residue was washed with MTBE (3 aliquots of 2 mL). This solid was used without further purification. Yield 35.5 mg, quantitative. Ions found by LCMS: [M+H]+=555.1.

### Step 3 (SEQ ID NO: 297)

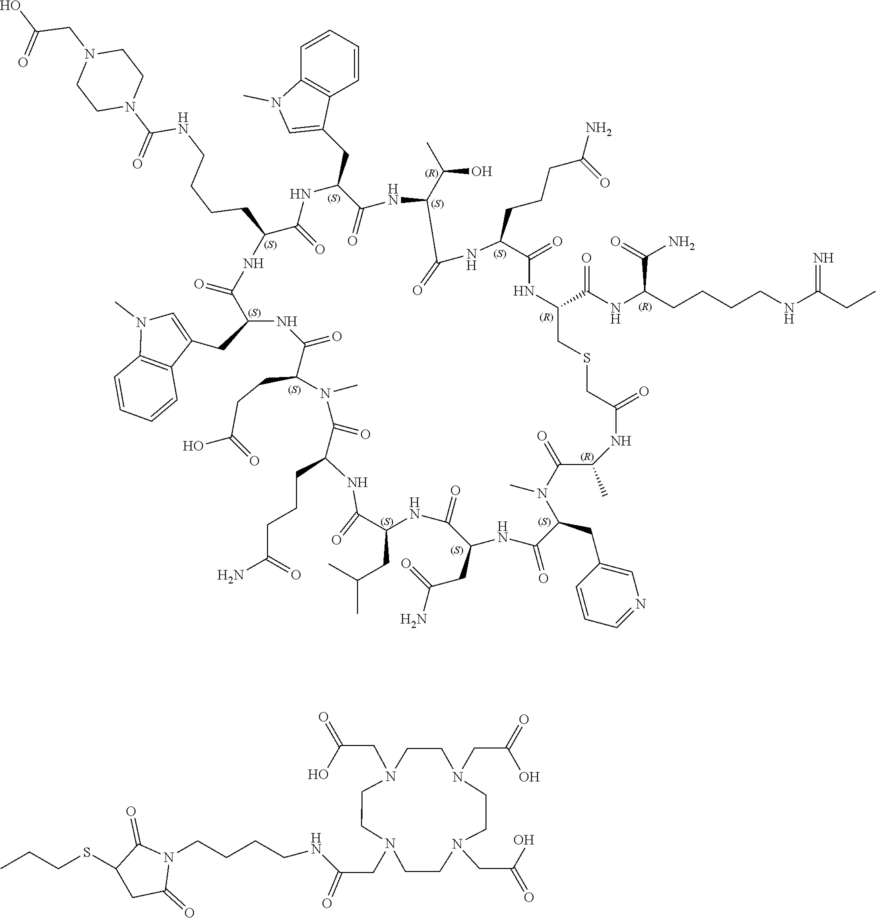

A solution of the product from step 1 (3 mg, 1.44 μmol) in 10× phosphate buffer at pH 7.4 (1 mL) was treated with a 100 mM solution at pH 7.4 of TCEP (10 μL) for 30 minutes. Subsequently, the product from step 2 (1.2 mg, 2.17 μmol) was added. Upon completion (LC-MS monitoring), the reaction was injected directly onto a prep HPLC column for purification (0 to 50% acetonitrile and water, using 0.1% TFA as modifier). Yield 3.0 mg, 79%. Ions found by LCMS: [M+H]⁺=2631.3, [(M+2H)/2]+=1316.1, [(M+3H)/3]+=877.8, [(M+4H)/4]+=658.6, [(M+5H)/5]+=527.1.

### Step 4 (SEQ ID NO: 297)

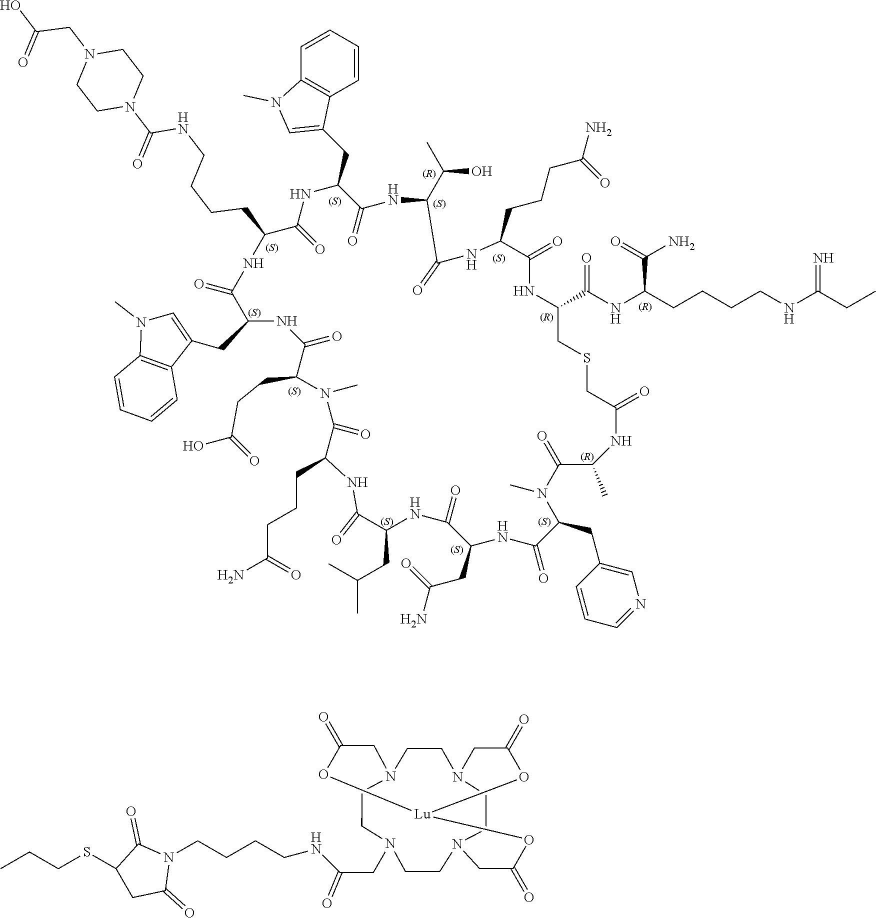

The product from step 3 (3 mg, 1.14 μmol) was dissolved in 250 μL of 0.4 M pH 5.5 sodium acetate buffer, and a water solution of Lutetium trichloride (96 μL of a 10 mg/mL solution, 3.42 μmol, 3.0 eqmol) was added to the solution. Upon full complexation (LC-MS monitoring), the reaction was injected directly onto a prep HPLC column for purification (0 to 50% acetonitrile and water, using 0.1% TFA as modifier). Yield 1.0 mg, 31%. Ions found by LCMS: [M+H]⁺=2806.2, [(M+2H)/2]+=1403.1, [(M+3H)/3]+=935.4, [(M+4H)/4]+=701.6, [(M+5H)/5]+=561.2.

### Example A9: Synthesis of Peptidyl-Resin 13

To the swollen Rink Amide-MBHA Resin (0.30 mmol, 0.32 mmol/g, 1.00 equiv) was removed via 20 min agitation with 20% piperidine in DMF followed by filtration and washing. Then the resin was added Dde-D-Lys (Fmoc)-OH (0.48 g, 0.9 mmol) HBTU (0.32 g, 0.85 mmol) and DIEA (0.23 g, 1.8 mmol) in dry DMF. The mixture was agitated for 30 min under nitrogen. After the reaction solution was removed through filtration, the resin was washed three times with DMF (10 mL). The Fmoc protecting group was removed via 30 min agitation with 20% piperidine in DMF followed by filtration and washing.

Subsequent amino acids were coupled using Fmoc-protected amino acid (3.00 equiv), HBTU (2.85 equiv) and DIEA (6.00 equiv) in dry DMF, shaking for 30 min. Pre-activation of any amino acid was not performed prior to coupling. Between amino acid couplings, the Fmoc protecting group was removed via 30 min agitation with 20% piperidine in DMF followed by filtration and washing. Success of Fmoc removal steps and amino acid couplings were monitored qualitatively using a ninhydrin test.

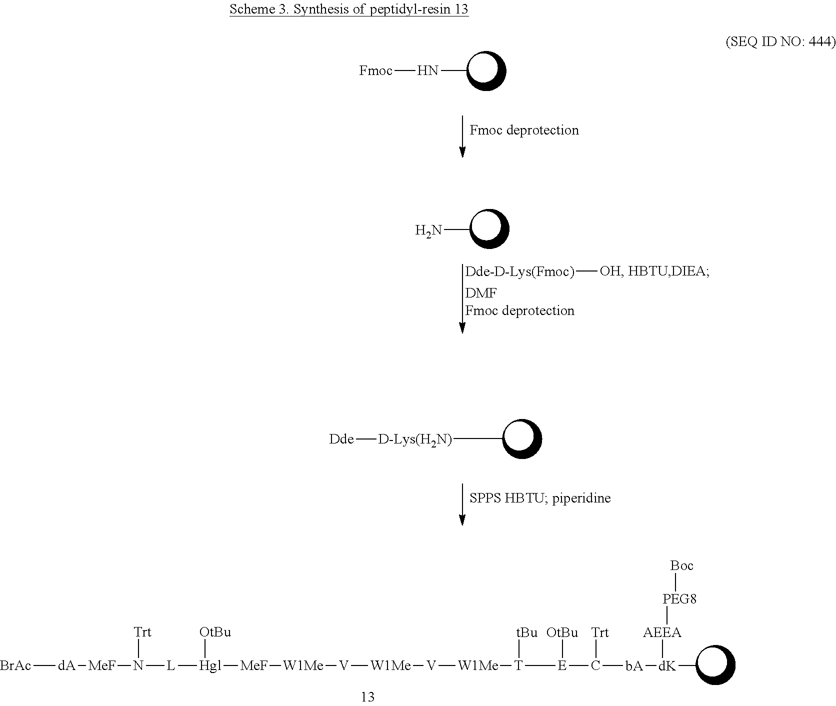

### Example A10: Synthesis of PDC_EphA2-00001417-C306

After the peptidyl-resin 13 was washed three times with MeOH and dried under vacuum, a cocktail of trifluoroacetic acid/H2O/triisopropylsilane/3-mercaptopropionic acid (90:2.5:2.5:5.0) was added. The resulting mixture was stirred for 2 h at room temperature. Cold isopropyl ether was added. The precipitated crude linear peptide-14 was collected through filtration and dried under vacuum.

Cyclization of peptide-14: To a solution of crude 14 (1.0 g) in water (350 mL) and MeCN (150 mL) were added Cs₂CO₃ (3.0 eq). The resulting mixture was stirred at room temperature for 0.5 hour. The pH of the solution was then adjusted to 5.0 using 1.0 N HCl. After lyophilization, the crude was purified by preparative HPLC to afford peptide-15 (200 mg) as a white solid.

To a solution of peptide-15 (200 mg) in DMF (3 mL) were added DOTA-OSu (1.5 eq), DIEA (3 eq). The resulting mixture was stirred at room temperature for 0.5 hour, the crude was purified by preparative HPLC to afford EphA2-00001417-C206 (121 mg, 14.3% yield, 99.34% purity) as a white solid.

Lu³⁺ complexation: To a solution of EphA2-00001417-C206 (100 mg, 99.34% purity, 35.0 μmol) in H₂O (5 mL) and MeCN (1 mL) was added LuCl₃ (12.6 mg, 45.0 μmol) and Na₂CO₃ (0.4 mg, 3.5 umol). The resulting mixture was stirred at 70 °C for 1 h. After filtration, the crude product was purified by preparative HPLC to afford EphA2-00001417-C306 (67.3 mg, 63.6% yield, 98.56% purity) as a white solid.

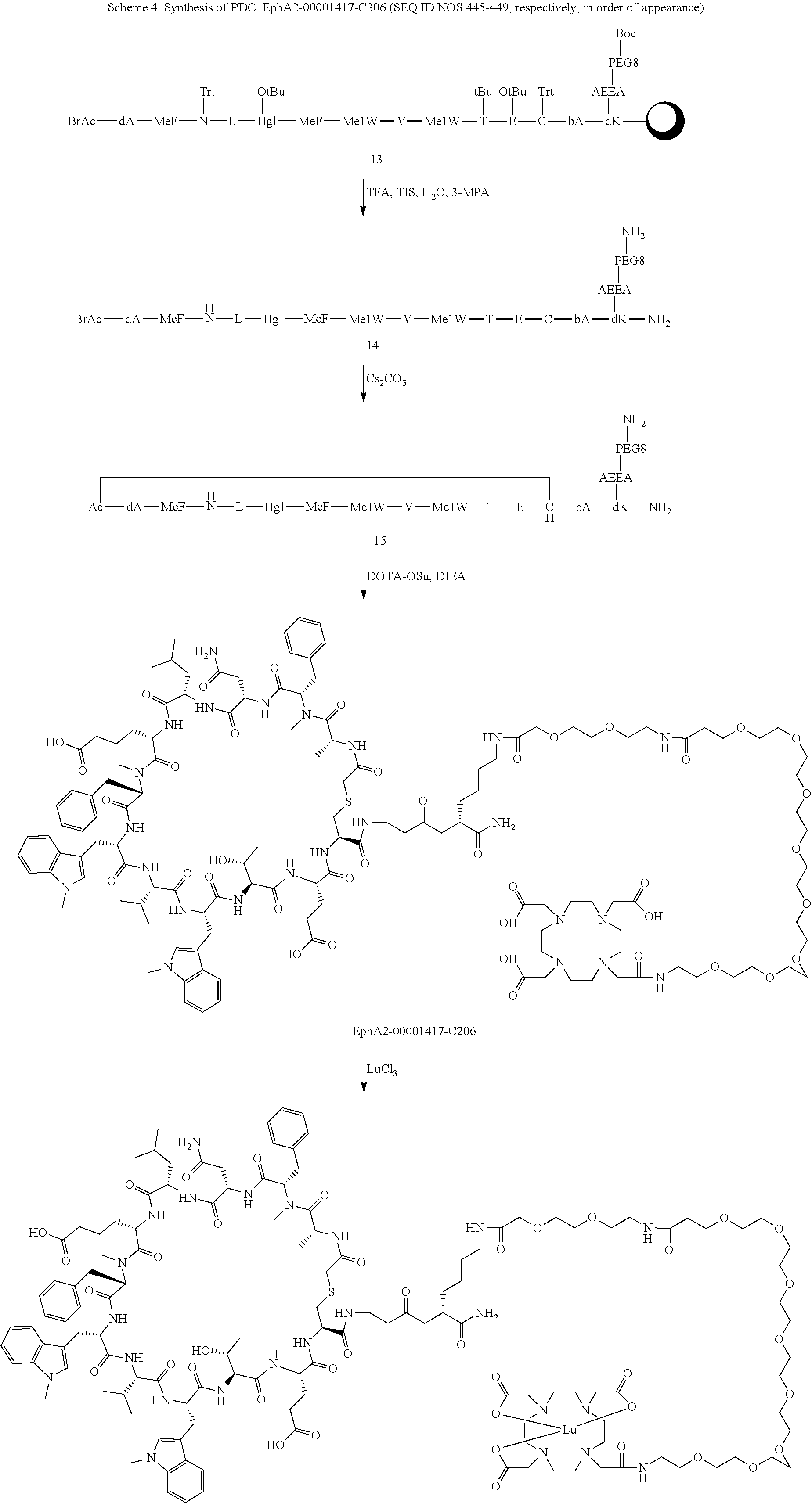

### EphA2-00001417-C306

### Example A11: Synthesis of 225-Actinium Chelated Conjugates

A peptide of the present disclosure is synthesized according to Example A2. The peptide is cyclized and coupled with a metal chelator (e.g., DOTA or other chelator described herein) according to example A3, optionally through a linker, thereby producing a conjugate comprising a cyclic peptide and a metal chelator.

General procedure for ²²⁵Ac-labeling [²²⁵Ac]Ac(NO₃)₃ in 1 mM HCl (100 μCi) is added to a mixture of a DOTA-cyclic peptide construct (4 nmol) in NaOAc buffer (0.4 M, pH 5.5-6.5, total volume 150 μL) in a 1.8 mL Eppendorf tube. The resulting mixture is heated at 80-100 °C in a thermal mixer at a shaking speed of 500 rpm for 15-30 min. Radiochemical purity is determined by iTLC.

### Example A12: Synthesis of ¹⁷⁷Lutetium Chelated Conjugates

A peptide of the present disclosure is synthesized according to Example A2. The peptide is cyclized and coupled with a metal chelator (e.g., DOTA or other chelator described herein) according to example A3, optionally through a linker, thereby producing a conjugate comprising a cyclic peptide and a metal chelator.

General procedure for ¹⁷⁷Lu-labeling [¹⁷⁷Lu]LuCl₃ in 0.04 M HCl (2.5 mCi) is added to a mixture of a DOTA-cyclic peptide construct (1.5 nmol) in NaOAc buffer (5 mg/mL Gentisic Acid, 0.4 M, pH 4.8-5.2, total volume 120 μL) in a 1.8 mL Eppendorf tube. The resulting mixture is heated at 80 °C in a thermal mixer at a shaking speed of 500 rpm for 30 min. If necessary, the mixture is purified using a C8 column. Radiochemical purity is determined by radio-RP-HPLC and iTLC.

### Example A13: Exemplary Radiolabeling Methods

Exemplary methods of labeling with a covalently bound radionuclide such as ¹³¹I are illustrated below.

### Reaction A13-1:

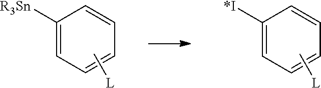

R₃=Bu₃, Me₃, ((CH₂)₂ (CF₂)₅CF₃)₃ or other alkyl groups, Rs may also be different e.g. SnR₃=Sn(Bu₂)(CH₂)₅-imidazole-Et PF₆. I*=¹²³⁴I, ¹²⁴I, ¹²⁵I, ¹³¹I; L=linker to binder (e.g., a peptide). L attached on various positions on aromatic ring.

### Reaction A13-2:

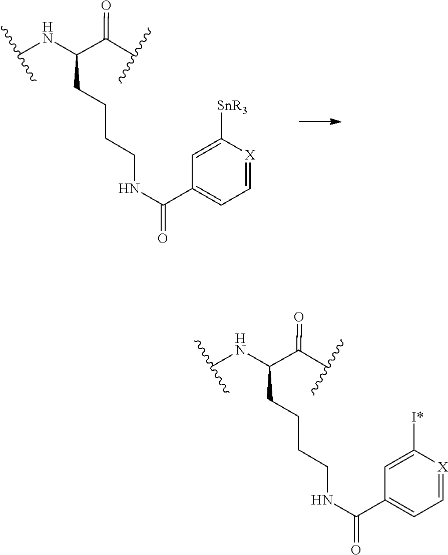

I*=¹²³⁴I, ¹²⁴I, ¹²⁵I, ¹³¹I; X=C—H, C—OH or N; Lys can be exchanged with other amino acids or amine containing groups.

### Reaction A13-3:

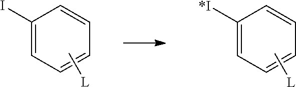

I*=¹²³⁴I, ¹²⁴I, ¹²⁵I, ¹³¹I; L=linker to binder (e.g. a peptide). L is attached on various positions on aromatic ring.

### Reaction A13-4:

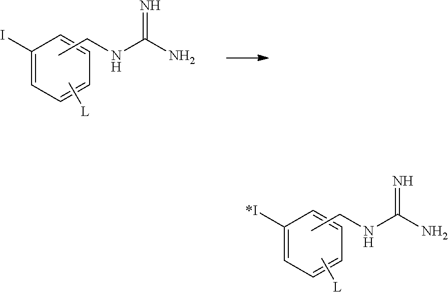

I*=¹²³⁴I, ¹²⁴I, ¹²⁵I, ¹³¹I; L=linker to binder; L and CH2NHCNHNH2 attached on various positions on the aromatic ring.

### Reaction A13-5:

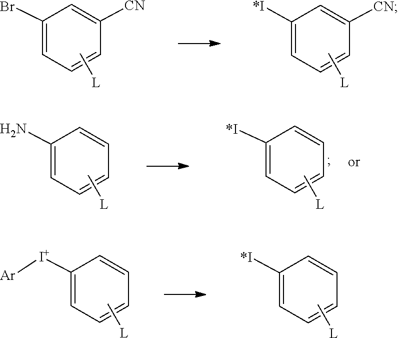

I*=¹²³⁴I, ¹²⁴I, ¹²⁵I, ¹³¹I; L=linker to binder; Ar=any aryl or hetero aryl group. L is attached at various positions on the aromatic ring.

### Reaction A13-6:

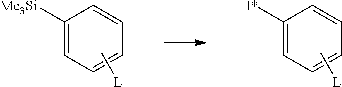

I*=¹²³⁴I, ¹²⁴I, ¹²⁵I, ¹³¹I; L=linker to binder. L is attached at various positions on the aromatic ring. SiMe3 can also be replaced with other Si-alkyl groups.

### Reaction A13-7:

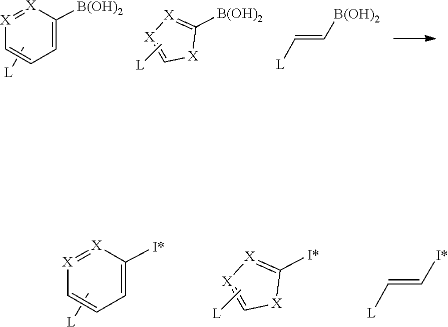

Each X is independently C, N, O, S. L=linker to binder. B(OH)² can also be replaced by B(OR)₂ where Rs are independent alkyl groups or form a ring. I*=¹²³⁴I, ¹²⁴I, ¹²⁵I, ¹³¹I.

### Reaction A13-8:

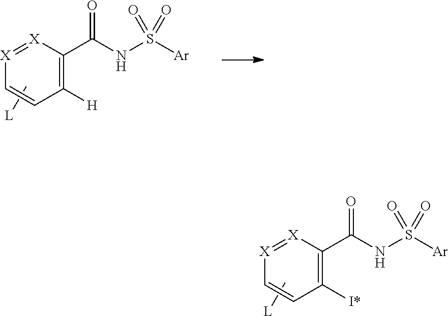

I*=¹²³⁴I, ¹²⁴I, ¹²⁵I, ¹³¹I; L=linker to binder; Ar=any aryl or hetero aryl group. Each X is independently C, N, O, S. L is attached at various positions on the aromatic ring.

### Reaction A13-9:

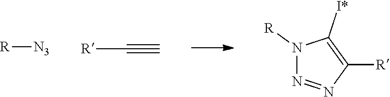

R or R′ may include a linker to binder. I*=¹²³⁴I, ¹²⁴I, ¹²⁵I, ¹³¹I.

### Reaction A13-10:

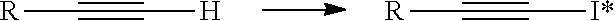

R is an alkyl, aryl or heteroaryl group and may include a linker to the binder; I*=¹²³⁴I, ¹²⁴I, ¹²⁵I, ¹³¹I.

### B: Synthesis of Peptides and Conjugates

### Example B1. Analytical Methods, Materials, and Instrumentation

Unless otherwise noted, purity and low-resolution mass spectral data of this section were measured using a Shimadzu LC/MS system or Waters ACQUITY LC/MS system (ESI). Methods are specified below.

General analytical Methods
Method A-A (HPLC-MS): Shimadzu LC/MS system, Kinetex EVO C18 2.6 μm, 2.1ID×150 mm, 100 Å (with a guard cartridge 2.1 mmID); 60 °C; 0.5 mL/min; (A) H2O+0.025% TFA/(B) MeCN+0.025% TFA; Gradient: from 5 to 45% B in 7.2 min. Electrospray mass spectra (+), PDA-UV chromatogram TIC, 225 nm.
Method A-B (HPLC-MS): Shimadzu LC/MS system, Kinetex EVO C18 2.6 μm, 2.1ID×150 mm, 100 Å (with a guard cartridge 2.1 mmID); 60 °C; 0.5 mL/min; (A) H2O+0.025% TFA/(B) MeCN+0.025% TFA; Gradient: from 20 to 60% B in 7.2 min. Electrospray mass spectra (+), PDA-UV chromatogram TIC, 225 nm.
Method A-C(HPLC-MS): Shimadzu LC/MS system, Kinetex EVO C18 2.6 μm, 2.1ID×150 mm, 100 Å (with a guard cartridge 2.1 mmID); 60 °C; 0.5 mL/min; (A) H2O+0.025% TFA/(B) MeCN+0.025% TFA; Gradient: from 40 to 80% B in 7.2 min. Electrospray mass spectra (+), PDA-UV chromatogram TIC, 225 nm.

### General Preparative HPLC Purification Procedure

The crude peptides were purified by preparative reverse phase C18-HPLC, using columns of different sizes and with varying flow rates, depending on the amount of crude peptide to be purified. Usually, 0.1% TFA in H₂O (A) and Acetonitrile (B) were employed as eluents. Product-containing fractions were collected and lyophilized to obtain the purified product.
Method P-A: XBridge C18 5 μm 50×150 mm; (20 mL/min-20 mL/min)/1 min, (20 mL/min-120 mL/min)/1 min, 120 mL/min for the rest.; (A) H2O+0.1% TFA/(B) MeCN+0.1% TFA; varying gradients; Detection: DAD-UV chromatogram TIC, 220 nm
Method P-B: XBridge C18 5 μm 50×150 mm; (20 mL/min-20 mL/min)/1 min, (20 mL/min-120 mL/min)/2 min, 120 mL/min for the rest.; (A) H2O+0.1% TFA/(B) MeCN+0.1% TFA; varying gradients; Detection: DAD-UV chromatogram TIC, 220 nm
Method P-C: XBridge C18 5 μm 50×150 mm; (120 mL/min-120 mL/min)/5 min, (20 mL/min-20 mL/min)/1 min, (20 mL/min-120 mL/min)/1 min, 120 mL/min for the rest.;
(A) H2O+0.1% TFA/(B) MeCN+0.1% TFA; varying gradients; Detection: DAD-UV chromatogram TIC, 220 nm
Method P-D: XBridge C18 5 μm 50×150 mm; 120 ml/min; (A) H2O+0.1% TFA/(B) MeCN+0.1% TFA; varying gradients; Detection: DAD-UV chromatogram TIC, 220 nm
Method P-E: XBridge C18 5 μm 50×250 mm; (118 mL/min-18 mL/min)/0.1 min, (18 mL/min-18 mL/min)/4.9 min, (18 mL/min-118 mL/min)/2 min, 118 mL/min for the rest.; (A) H2O+0.1% TFA/(B) MeCN+0.1% TFA; varying gradients; Detection: DAD-UV chromatogram TIC, 220 nm

### General Scheme A

Macrocyclic peptides Scheme A-I in the present disclosure can be synthesized by the general method outlined in scheme A.

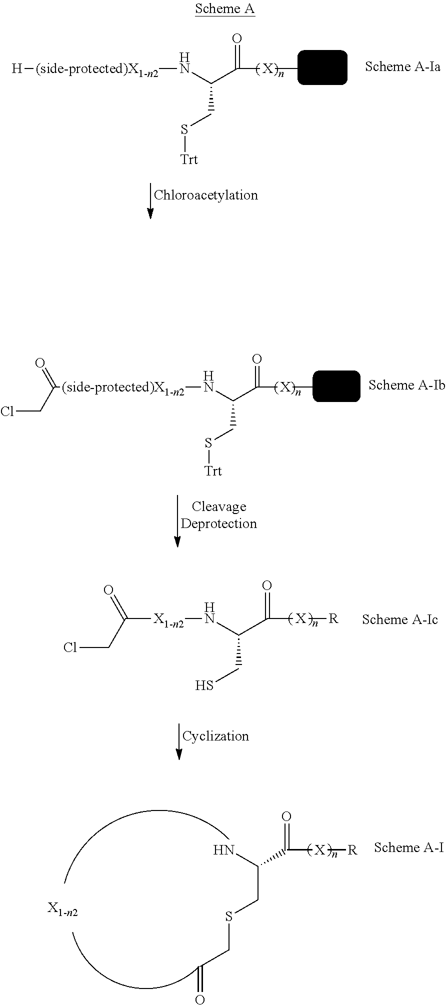

### Step 1: Solid Phase Peptide Synthesis (SPPS) Method A:

The peptide synthesis in this disclosure was performed on the Liberty BLUE HT 12TM (CEM. Inc.) according to the manufacturer's instruction. In fact, Fmoc-Sieber amide Resin was suspended in the solvent (e.g., DMF or DCM) and then loaded onto the peptide synthesizer. After the Fmoc removal of Fmoc-Sieber amide Resin, the coupling steps and Fmoc removal steps were repeatedly continued until the desired linear polypeptide of Scheme A-Ia was obtained. AA coupling and Fmoc deprotection conditions for method A were listed in the table below.

### General Method for AA Coupling

TABLE B1-1

| AA | Reagents | coupling conditions |
|---|---|---|
| Fmoc-Cys(Trt)-OH | AA/DIC/Oxyma | 50 °C, 1-2x 15-20 min |
| Other than | Pure ® in DMF | 90 °C, 1-2x 3-10 min |
| listed above | | or 75 °C, 2x 20-30 min |

The AAs listed below were introduced by employing DIC/Oxyma or HATU/DIEA.

TABLE B1-2

| AA | HATU | | DIEA |
|---|---|---|---|
| Fmoc-Me3Py—OH 4.2 eq. | 4-5 | eq. | 8-12 eq. |
| Fmoc-W1Me7Cl—OH 2.5-4.2 eq. | 2.5-5 | eq. | 5-10 eq. |
| Fmoc-df3CON—OH 4.2-5.3 eq. | 4-5 | eq. | 8-10 eq. |

### General Method for Fmoc Deprotection

TABLE B1-3

| AA | Reagents | deprotection conditions |
|---|---|---|
| AA next | pyrrolidine/DMF (1/9) | 25 °C, 1-2x 1-5 min |
| to N—Me AA | | |
| Other than | or piperidine/ | 90 °C, 1 min or |
| listed above | DMF (1/4) | 75 °C, 3 min |

### Step 1: Solid Phase Peptide Synthesis (SPPS) Method B:

The peptide synthesis in this disclosure was performed on the Liberty BLUE HT 12TM (CEM. Inc.) according to the manufacturer's instruction. In fact, Fmoc-Sieber amide Resin was suspended in the solvent (e.g., DMF or DCM) and then loaded onto the peptide synthesizer. After the Fmoc removal of Fmoc-Sieber amide Resin, the coupling steps and Fmoc removal steps were repeatedly continued until the desired linear polypeptide of Scheme A-Ia was obtained. AA coupling and Fmoc deprotection conditions for method B were listed in the table below.

### General Method for AA Coupling

TABLE B2-1

| AA | Reagents | coupling conditions |
|---|---|---|
| Fmoc-Cys(Trt)-OH | AA/DIC/Oxyma | 50 °C, 1-2x 15-20 min |
| Other than | Pure ® in DMF | 105 °C, 1-2x 2 min |
| listed above | | or 90 °C, 1-2x 3-10 min |
| | | or 75 °C, 2x 20-30 min |

### General Method for Fmoc Deprotection

TABLE B2-2

| AA | Reagents | deprotection conditions |
|---|---|---|
| AA next | pyrrolidine/DMF (1/9) | 25 °C, 1-2x 1-5 min |
| to N—Me AA | | |
| Other than | 83 mM Oxyma pure in | 110 °C, 1-1.5 min |
| listed above | pyrrolidine/DMF (4/96) | |

### Step 2: The Introduction of Chloroacetyl Group:

The obtained resin in the step-1 on the resin was transferred in a syringe with a flit. The resin was shaken in one of the reagents listed below at room temperature for 0.5-2 h. The solution was then drained through the frit. The resin was washed successively a few times with DMF, CH2Cl2, and Et2O to afford the linear peptides of Scheme A-Ib on the resin.

Chloroacetylation Conditions were Listed in the Table Below.

TABLE B3-1

| | |
|---|---|
| ClAc-1 | resin/ClAcOH in DMF(0.1-0.2M)/HATU |
| | in DMF(0.1-0.5M)/DIPEA in DMF (0.2-1M) |
| | (1 eq/4.2-5 eq/4-5 eq/4.2-10 eq) |
| ClAc-2 | ClAcOSu NMP/DCM (0.1-0.2M) prepared by |
| | ClAcOH/DIC/HOSu (4-10 eq/4-10 eq/4-10 |
| | eq to 1 eq of resin) |
| ClAc-3 | ClAcOSu in DMF/DCM (0.1-0.25M) prepared |
| | by ClAcOH/DIC/HOSu (4-10 eq/4-10 eq/4-10 |
| | eq to 1 eq of resin) |
| ClAc-4 | ClAcOSu in DMF (4-10 eq to 1 eq of resin) |

Step 3: Cleavage from Resin and Global Deprotection of Side Chain Protecting Groups (PGs)

The intermediate Scheme A-Ib was shaken at rt for 20-90 min with the cleavage cocktail solution listed below. The resin was filtered and washed with the cleavage solution. The resin was then filtered, and the combined filtrates were poured into cold diethyl ether. The resulting suspension was centrifuged (9000 rpm, 1 min at 0 °C) and the supernatant was decanted out. The precipitate was suspended in cold ether, vortexed briefly, and then centrifuged. This process was repeated when appropriate. The crude containing peptide Scheme A-Ic was dried under reduced pressure.

Cleavage Conditions were Listed in the Table Below.

TABLE B4

| | |
|---|---|
| Cocktail-A | TFA/TIS/DODT/H₂O = 92.5/2.5/2.5/2.5 |
| Cocktail-B | TFA/TIS/DODT/H₂O = 90/2.5/5.0/2.5 |

### Step 4: Peptide Cyclization

The crude containing polypeptide Scheme A-Ic was dissolved in one of solvents listed below, and subsequently added TEA (5-15 eq). The mixture was then shaken at room temperature for 1 h to ca. 16 h until completion of the reaction. The resulting reaction mixture was concentrated by Genevac E-2 Elite or lyophilization. The resulting residue containing the desired polypeptide was purified by preparative reverse phase C18-HPLC to afford Scheme A-I polypeptides.

Peptide cyclization conditions were listed in the table below.

TABLE B5

| | Solvent | Reagent | Time |
|---|---|---|---|
| Cy-1 | DMSO/H₂O (95/5) | TEA (5-15 eq) | 1-16 h |
| Cy-2 | MeCN/H₂O (1/1) | | |
| Cy-3 | DMSO/H₂O (9/1) | | |
| Cy-4 | DMSO | | |
| Cy-5 | DMSO/H₂O/IPA (90/5/5) | | |
| Cy-6 | DMSO/MeCN/H2O (1/1/1) | | |

### General Scheme B

Macrocyclic peptides Scheme B-I in the present disclosure can be synthesized on the pre-loaded resin in the analogous manner to the scheme A. The general synthetic scheme is described in Scheme B shown below.

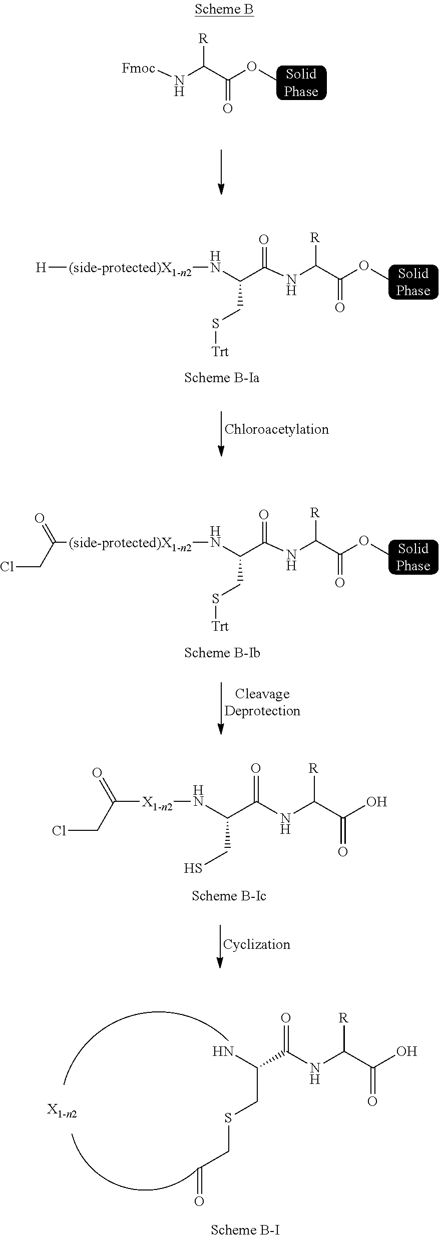

### General Scheme C

Macrocyclic peptides Scheme C-I in the present disclosure can be synthesized by following the steps outlined in scheme C shown below.

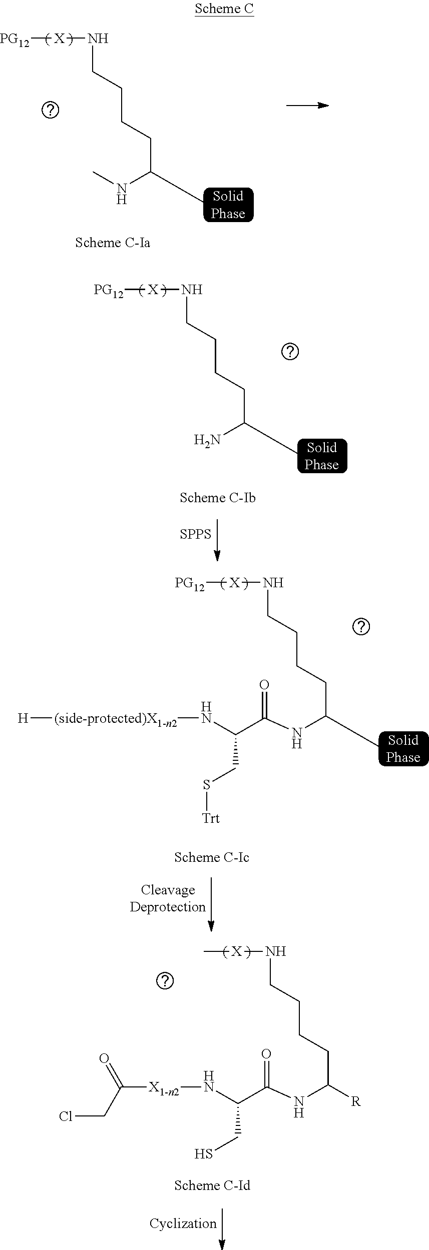

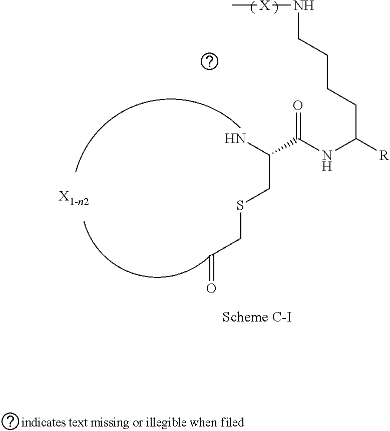

### Step 1: Peptide Synthesis

Intermediates Scheme C-Ia on-resin were synthesized in the analogous manner to the step 1 of the scheme A.

### Step 2: Deprotection of Alloc Protecting Group

The polypeptides Scheme C-Ia on-resin were suspended in DCM. The resin was shaken with Pd(PPh3)4 (0.2-0.25 eq) and PhSiH3 (10-15 eq) at room temperature for 1 h. The resin was washed with DMF. Then the resin was washed by DCM followed by DMF to provide the intermediate polypeptides Scheme C-Ib on the resin.

### Step 3-5:

Polypeptides of formula Scheme C-I were synthesized from the intermediates Scheme C-Ib on-resin in the analogous manner to scheme A.

### Example B2: PDC_EphA2-00007196-C002 (SEQ ID No: 200)

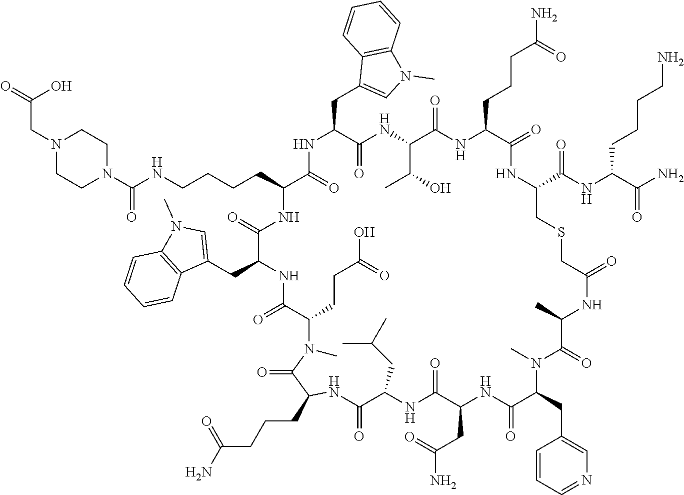 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(N)=O)NC1=O]

The peptide sequence was synthesized on Fmoc-Sieber amide Resin (0.375 mmol) following general peptide synthesis method A. The obtained peptide on the resin was subjected to the general method ClAc-1. The polypeptide on the resin was treated with Cleavage Cocktail-A (18 mL) to furnish the linear peptide. The crude containing the linear peptide (0.375 mmol as theoretical based on the resin used) was subjected to the peptide cyclization condition-3. The resulting residue was purified by preparative reversed-phase HPLC (Method P-A). Pure fractions were combined and lyophilized to afford the title macrocyclic peptide. HPLC-MS (Method A-A): 5.16 min, ESI-MS m/z: 988.9 [M+2H]2+, AUC (UV 225 nm): 94%.

### Example B3: PDC_EphA2-00008093-C002 (SEQ ID No: 199)

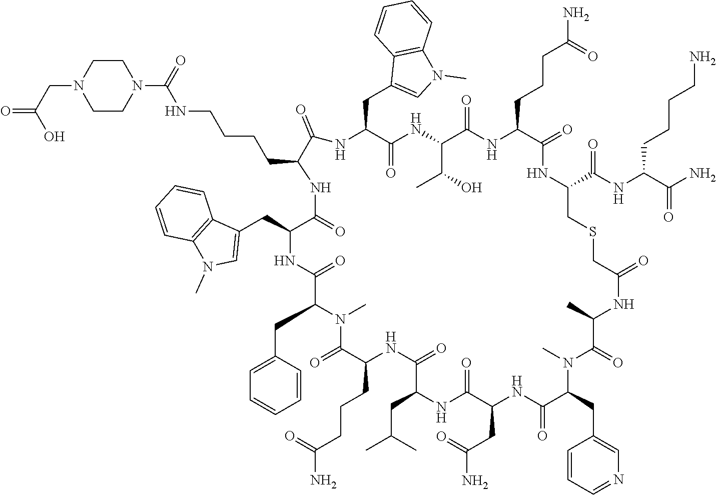 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(N)=O)NC1=O]

The peptide sequence was synthesized on Fmoc-Sieber amide Resin (0.375 mmol) following general peptide synthesis method A. The obtained peptide on the resin was subjected to the general method ClAc-1. The polypeptide on the resin was treated with Cleavage Cocktail-A (18 mL) to furnish the linear peptide. The crude containing the linear peptide (0.375 mmol as theoretical based on the resin used) was subjected to the peptide cyclization condition-3. The resulting residue was purified by preparative reversed-phase HPLC (Method P-A). Pure fractions were combined and lyophilized to afford the title macrocyclic peptide. HPLC-MS (Method A-B): 3.51 min, ESI-MS m/z: 998.0 [M+2H]2+, AUC (UV 225 nm): 97%.

### Example B4: PDC_EphA2-00019437-C002 (SEQ ID No: 204)

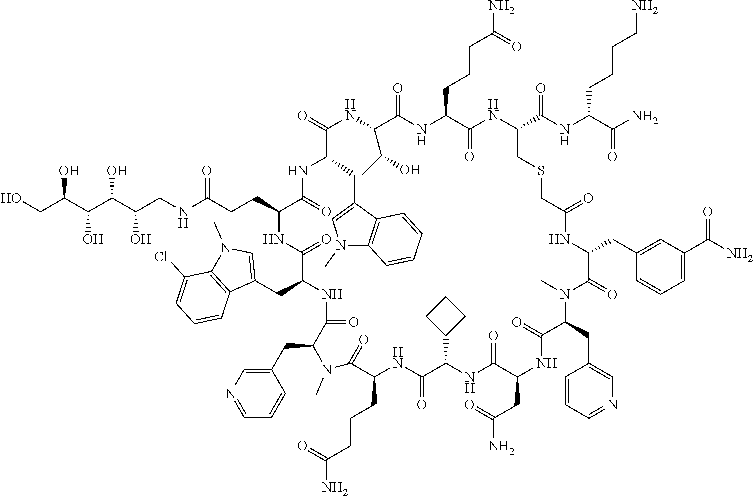 [SMILES: C[C@@H](O)[C@@H]1NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3c(Cl)cccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(=O)N[C@H](CCCCN)C(N)=O)NC(=O)[C@H](CCCC(N)=O)NC1=O]

The peptide sequence was synthesized on Fmoc-Sieber amide Resin (0.57 mmol/g, 0.25 mmol) following general peptide synthesis method A. The obtained peptide on the resin was subjected to the general method ClAc-2. The polypeptide on the resin was treated with Cleavage Cocktail-A (15 mL) to furnish the linear peptide. The crude containing the linear peptide (0.25 mmol as theoretical based on the resin used) was subjected to the peptide cyclization condition-3. The resulting residue was purified by preparative reversed-phase HPLC (Method P-A). Pure fractions were combined and lyophilized to afford the title macrocyclic peptide. HPLC-MS (Method A-A): 5.07 min, ESI-MS m/z: 1071.6 [M+2H]2+, AUC (UV 225 nm): 98%.

### Example B5: PDC_EphA2-00019440-C002 (SEQ ID No: 208)

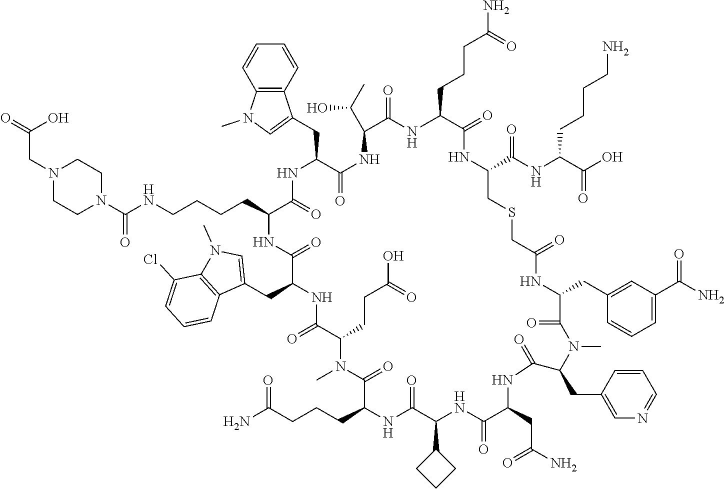 [SMILES: C[C@@H](O)[C@@H]1NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@H](CCC(=O)O)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(=O)N[C@H](CCCCN)C(=O)O)NC(=O)[C@H](CCCC(N)=O)NC1=O]

The peptide sequence was synthesized on Fmoc-Sieber amide Resin (0.25 mmol) following general peptide synthesis method A. The obtained peptide on the resin was subjected to the general method ClAc-3. The polypeptide on the resin was treated with Cleavage Cocktail-B (15 mL) to furnish the linear peptide. The crude containing the linear peptide (0.25 mmol as theoretical based on the resin used) was subjected to the peptide cyclization condition-3. The resulting residue was purified by preparative reversed-phase HPLC (Method P-A). Pure fractions were combined and lyophilized to afford the title macrocyclic peptide. HPLC-MS (Method A-A): 5.44 min, ESI-MS m/z: 1065.1 [M+2H]2+, AUC (UV 225 nm): 99%.

### Example B6

The following Examples were prepared by following similar methods described in the examples above. The amino acid sequence for each peptide was shown in Table B6 and Table B6-1. The term “Term” means the functional group at C-terminus of the peptide.

TABLE B6

| SEQ | | | | | | | | | | | | |
|---|---|---|---|---|---|---|---|---|---|---|---|---|
| ID | Sequence | | | | | | | | | | | |
| No. | 1 | 2 | 3 | 4 | 5 | 6 | 7 | 8 | 9 | 10 | 11 | 12 |
| 1 | da | MeF | N | L | Hgl | MeF | W1Me | V | W1Me | T | E | C |
| 2 | da | Me3Py | N | L | Hgn | MeF | W1Me | KCOpipzaa | W1Me | T | Hgn | C |
| 3 | da | Me3Py | N | Cbg | Hgn | MeF | W1Me | KCOpipzaa | W1Me | T | Hgn | C |
| 4 | da | Me3Py | N | Cbg | Hgn | N | W1Me | KCOpipzaa | W1Me | T | Hgn | C |
| 5 | da | Me3Py | N | Cbg | Hgn | MeN | W1Me | KCOpipzaa | W1Me | T | Hgn | C |
| 6 | da | Me3Py | N | Cbg | Hgn | MeF | W1Me7N | KCOpipzaa | W1Me | T | Hgn | C |
| 7 | da | Me3Py | N | Cbg | Hgn | MeF | W1Me | KCOpipzaa | F23dMe | T | Hgn | C |
| 8 | da | Me3Py | N | Cbg | Hgn | MeF | W1Me | KCOpipzaa | W1Me | T | N | C |
| 9 | da | Me3Py | N | Chg | Hgn | MeF | W1Me | KCOpipzaa | W1Me | T | Hgn | C |
| 10 | da | Me3Py | N | L | Hgl | MeE | W1Me | KCOpipzaa | W1Me | T | Hgn | C |
| 11 | da | Me3Py | N | Cbg | Hgl | MeE | W1Me | KCOpipzaa | W1Me | T | Hgn | C |
| 12 | da | Me3Py | N | L | Hgn | MeE | W1Me | KCOpipzaa | W1Me | T | Hgn | C |
| 13 | da | Me3Py | N | Cbg | Hgn | MeE | W1Me | KCOpipzaa | W1Me | T | Hgn | C |
| 14 | da | Me3Py | N | Cbg | Hgn | N | W1Me | V | W1Me | T | Hgn | C |
| 15 | da | Me3Py | N | Cbg | Hgn | MeN | W1Me | V | W1Me | T | Hgn | C |
| 16 | da | Me3Py | N | Cbg | Hgn | Me3Py | W1Me | V | W1Me | T | Hgn | C |
| 17 | da | MeF | N | Cbg | Hgn | MeN | W1Me | V | W1Me | T | Hgn | C |
| 18 | da | MeF | N | Cbg | Hgn | MeN | W1Me | Qglucamine | W1Me | T | Hgn | C |
| 19 | da | MeF | N | Cbg | Hgn | Me3Py | W1Me | Qglucamine | W1Me | T | Hgn | C |
| 20 | da | Me3Py | N | Cbg | Hgn | Me3Py | W1Me | Qglucamine | W1Me | T | Hgn | C |
| 21 | da | MeF | N | Cbg | Hgn | MeF | W1Me | Qglucamine | W1Me | T | Hgn | C |
| 22 | dahp | Me3Py | N | L | Hgl | MeF | W1Me | KCOpipzaa | W1Me | T | Hgn | C |
| 23 | da | Me3Py | N | L | Hgl | MeF4C | W1Me | KCOpipzaa | W1Me | T | Hgn | C |
| 24 | da | Me3Py | N | L | Hgl | MeF | W1Me | KCOpipzaa | W1Me | T | Nmm | C |
| 25 | da | Me3Py | N | L | Hgl | MeF | W1Me | KCOpipzaa | W1Me | T | Ndm | C |
| 26 | dahp | Me3Py | N | L | Hgn | MeE | W1Me | KCOpipzaa | W1Me | T | Hgn | C |
| 27 | da | Me3Py | N | L | Nmm | MeE | W1Me | KCOpipzaa | W1Me | T | Hgn | C |
| 28 | da | Me3Py | N | L | Ndm | MeE | W1Me | KCOpipzaa | W1Me | T | Hgn | C |
| 29 | da | Me3Py | N | L | Hgn | MeE | W1Me | KCOpipzaa | W1Me | T | Nmm | C |
| 30 | da | Me3Py | N | L | Hgn | MeE | W1Me | KCOpipzaa | W1Me | T | Ndm | C |
| 31 | dahp | Me3Py | N | Chg | Hgn | MeF | W1Me | KCOpipzaa | W1Me | T | Hgn | C |
| 32 | da | Me3Py | N | Chg | Nmm | MeF | W1Me | KCOpipzaa | W1Me | T | Hgn | C |
| 33 | da | Me3Py | N | Chg | Ndm | MeF | W1Me | KCOpipzaa | W1Me | T | Hgn | C |
| 34 | da | Me3Py | N | Chg | Hgn | MeF4C | W1Me | KCOpipzaa | W1Me | T | Hgn | C |
| 35 | da | Me3Py | N | Chg | Hgn | MeF | W1Me | KCOpipzaa | W1Me | T | Nmm | C |
| 36 | da | Me3Py | N | Chg | Hgn | MeF | W1Me | V | W1Me | T | Ndm | C |
| 37 | df3CON | MeF | N | Cbg | Hgn | MeN | W1Me7Cl | V | W1Me | T | Hgn | C |
| 38 | df3CON | MeF | N | L | Hgn | MeN | W1Me7Cl | V | W1Me | T | Hgn | C |
| 39 | df3CON | MeF | N | L | Hgn | MeN | W1Me | V | W1Me | T | Hgn | C |
| 40 | df3CON | Me3Py | N | Cbg | Hgn | MeN | W1Me7Cl | V | W1Me | T | Hgn | C |
| 41 | df3CON | MeF | N | Cbg | Hgn | MeN | W1Me7Cl | KCOpipzaa | W1Me | T | Hgn | C |
| 42 | df3CON | MeF | N | Cbg | Hgn | MeN | W1Me7Cl | Hcit | W1Me | T | Hgn | C |
| 43 | df3CON | MeF | N | Cbg | Hgn | MeE | W1Me7Cl | Hcit | W1Me | T | Har | C |
| 44 | df3CON | Me3Py | N | Cbg | Hgn | MeN | W1Me7Cl | Hcit | W1Me | T | R | C |
| 45 | df3CON | Me3Py | N | Cbg | Hgn | MeN | W1Me7Cl | Hcit | W1Me | T | Har | C |
| 46 | df3CON | Me3Py | N | Cbg | Hgn | MeN | W1Me7Cl | KCOpipzaa | W1Me | T | Har | C |
| 47 | df3CON | MeF | N | Cbg | Hgn | MeF | W1Me7Cl | Qglucamine | W1Me | T | Hgn | C |
| 48 | df3CON | MeF | N | Cbg | Hgn | MeF | W1Me7Cl | KCOpipzaa | W1Me | T | Hgn | C |
| 49 | df3CON | Me3Py | N | Cbg | Hgn | MeF | W1Me7Cl | Qglucamine | W1Me | T | Hgn | C |
| 50 | df3CON | Me3Py | N | Cbg | Hgn | MeF | W1Me7Cl | KCOpipzaa | W1Me | T | Hgn | C |
| 51 | df3CON | MeF | N | Cbg | Hgn | Me3Py | W1Me7Cl | Qglucamine | W1Me | T | Hgn | C |
| 52 | df3CON | MeF | N | Cbg | Hgn | Me3Py | W1Me7Cl | KCOpipzaa | W1Me | T | Hgn | C |
| 53 | df3CON | Me3Py | N | Cbg | Hgn | Me3Py | W1Me7Cl | Qglucamine | W1Me | T | Hgn | C |
| 54 | df3CON | Me3Py | N | Cbg | Hgn | Me3Py | W1Me7Cl | KCOpipzaa | W1Me | T | Hgn | C |
| 55 | df3CON | Me3Py | N | Cbg | Hgn | MeE | W1Me7Cl | Qglucamine | W1Me | T | Hgn | C |
| 56 | df3CON | Me3Py | N | Cbg | Hgn | MeE | W1Me7Cl | KCOpipzaa | W1Me | T | Hgn | C |
| 57 | df3CON | Me3Py | N | Cbg | KCOpipzaa | MeF | W1Me7Cl | KCOpipzaa | W1Me | T | Hgn | C |
| 58 | df3CON | Me3Py | N | Cbg | KCOpipzaa | MeF | W1Me7Cl | Qglucamine | W1Me | T | Hgn | C |
| 59 | df3CON | Me3Py | N | L | N | Me3Py | W1Me7Cl | Qglucamine | W1Me | T | N | C |
| 60 | df3CON | Me3Py | N | L | N | MeN | W1Me7Cl | Qglucamine | W1Me | T | N | C |
| 61 | df3CON | Me3Py | N | L | N | MeN | W1Me7Cl | Cit | W1Me | T | N | C |
| 62 | da | Me3Py | N | L | K | MeE | W1Me7Cl | KCOpipzaa | W1Me | T | Hgn | C |
| 63 | da | Me3Py | N | L | Hgn | MeE | W1Me7Cl | K | W1Me | T | Hgn | C |
| 64 | da | Me3Py | N | L | Hgn | MeE | W1Me7Cl | KCOpipzaa | W1Me | T | K | C |
| 65 | df3CON | Me3Py | N | L | K | MeN | W1Me7Cl | Qglucamine | W1Me | T | Hgn | C |
| 66 | df3CON | Me3Py | N | L | Hgn | MeN | W1Me7Cl | K | W1Me | T | Hgn | C |
| 67 | df3CON | Me3Py | N | L | Hgn | MeN | W1Me7Cl | Qglucamine | W1Me | T | K | C |
| 68 | df3CON | Me3Py | N | L | K | MeE | W1Me7Cl | KCOpipzaa | W1Me | T | Hgn | C |
| 69 | df3CON | Me3Py | N | L | Hgn | MeE | W1Me7Cl | K | W1Me | T | Hgn | C |
| 70 | df3CON | Me3Py | N | L | Hgn | MeE | W1Me7Cl | KCOpipzaa | W1Me | T | K | C |
| 71 | da | Me3Py | N | Cbg | Hgn | MeN | W1Me | V | W1Me | T | Hgn | C |
| 72 | da | Me3Py | N | Cbg | Hgn | Me3Py | W1Me | V | W1Me | T | Hgn | C |
| 73 | da | MeF | N | Cbg | Hgn | MeN | W1Me | V | W1Me | T | Hgn | C |
| 74 | da | MeF | N | L | Hgl | MeF | W1Me | V | W1Me | T | E | C |
| 75 | da | Me3Py | N | L | Hgl | MeF | W1Me | V | W1Me | T | E | C |
| 76 | KCOpipzaa | Me3Py | N | L | Hgl | MeF | W1Me | V | W1Me | T | E | C |
| 77 | KCOpipzaa | Me3Py | N | L | KCOpipzaa | MeF | W1Me | V | W1Me | T | E | C |
| 78 | KCOpipzaa | Me3Py | N | L | KCOpipzaa | MeF | W1Me | V | W1Me | T | Hgn | C |
| 79 | da | Me3Py | N | L | Hgl | MeF | W1Me | KCOpipzaa | W1Me | T | E | C |
| 80 | da | Me3Py | N | L | KCOpipzaa | MeF | W1Me | KCOpipzaa | W1Me | T | E | C |
| 81 | da | Me3Py | N | L | KCOpipzaa | MeF | W1Me | KCOpipzaa | W1Me | T | E | C |
| 82 | da | Me3Py | N | L | KCOpipzaa | MeF | W1Me | KCOpipzaa | W1Me | T | E | C |
| 83 | da | Me3Py | N | L | Hgl | MeF | W1Me | KCOpipzaa | W1Me | T | Hgn | C |
| 84 | da | Me3Py | N | L | KCOpipzaa | MeF | W1Me | KCOpipzaa | W1Me | T | Hgn | C |
| 85 | da | Me3Py | N | L | Hgn | MeF | W1Me | KCOpipzaa | W1Me | T | Hgn | C |
| 86 | da | Me3Py | N | L | Hgl | MeE | W1Me | KCOpipzaa | W1Me | T | Hgn | C |
| 87 | da | Me3Py | N | L | KCOpipzaa | MeE | W1Me | KCOpipzaa | W1Me | T | Hgn | C |
| 88 | da | Me3Py | N | L | Hgn | MeE | W1Me | KCOpipzaa | W1Me | T | Hgn | C |
| 89 | A | Me3Py | N | L | Hgl | MeF | W1Me | KCOpipzaa | W1Me | T | E | C |
| 90 | da | Me3Py | N | L | Hgn | MeF | W1Me | KCOpipzaa | W1Me | T | Hgn | C |
| 91 | da | Me3Py | N | L | Hgn | MeE | W1Me | KCOpipzaa | W1Me | T | Hgn | C |
| 92 | da | Me3Py | N | Cbg | Hgn | MeN | W1Me | V | W1Me | T | Hgn | C |
| 93 | da | Me3Py | N | Cbg | Hgn | Me3Py | W1Me | V | W1Me | T | Hgn | C |
| 94 | da | MeF | N | Cbg | Hgn | MeN | W1Me | V | W1Me | T | Hgn | C |
| 95 | df3CON | Me3Py | N | Cbg | Hgn | Me3Py | W1Me7Cl | Qglucamine | W1Me | T | Hgn | C |
| 96 | df3CON | Me3Py | N | Cbg | Hgn | MeN | W1Me7Cl | Qglucamine | W1Me | T | Hgn | C |
| 97 | df3CON | Me3Py | N | Cbg | Qglucamine | MeN | W1Me7Cl | Qglucamine | W1Me | T | Hgn | C |
| 98 | df3CON | Me3Py | N | Cbg | Hgn | MeN | W1Me | Qglucamine | W1Me | T | Hgn | C |
| 99 | df3CON | Me3Py | N | Cbg | Hgn | MeE | 7W1MeC1 | KCOpipzaa | W1Me | T | Hgn | C |
| 100 | df3CON | MeF3CN | N | Cbg | Hgn | MeE | W1Me7Cl | KCOpipzaa | W1Me | T | Hgn | C |
| 101 | df3CON | MeF3H | N | Cbg | Hgn | MeE | W1Me7Cl | KCOpipzaa | W1Me | T | Hgn | C |
| 102 | df3CON | MeF3CN | N | Cbg | Hgn | Me3Py | W1Me7Cl | Qglucamine | W1Me | T | Hgn | C |
| 103 | df3CON | MeF3CN | N | Cbg | Hgn | MeN | W1Me7Cl | Qglucamine | W1Me | T | Hgn | C |
| 104 | da | MeF | N | Cbg | Hgn | MeN | W1Me | Qglucamine | W1Me | T | Hgn | C |
| 105 | da | MeF | N | Cbg | Hgn | Me3Py | W1Me | Qglucamine | W1Me | T | Hgn | C |
| 106 | da | Me3Py | N | Cbg | Hgn | Me3Py | W1Me | Qglucamine | W1Me | T | Hgn | C |
| 107 | df3CON | Me3Py | N | L | N | Me3Py | W1Me7Cl | Qglucamine | W1Me | T | N | C |
| 108 | df3CON | Me3Py | N | L | N | MeN | W1Me7Cl | Qglucamine | W1Me | T | N | C |
| 109 | df3CON | Me3Py | N | L | N | MeN | W1Me7Cl | Cit | W1Me | T | N | C |
| 110 | da | Me3Py | N | L | Hgn | MeE | W1Me | KCOpipzaa | W1Me | T | Hgn | C |
| 111 | da | MeF | N | Cbg | Hgn | MeN | W1Me | V | W1Me | T | Hgn | C |
| 112 | df3CON | Me3Py | N | Cbg | Hgn | Me3Py | W1Me7Cl | Qglucamine | W1Me | T | Hgn | C |
| 113 | df3CON | MeF3H | N | Cbg | Hgn | MeE | W1Me7Cl | KCOpipzaa | W1Me | T | Hgn | C |
| 114 | da | MeF | N | L | Hgl | MeF | W1Me | V | W1Me | T | E | C |
| 115 | da | Me3Py | N | L | Hgn | MeE | W1Me | KCOpipzaa | W1Me | T | Hgn | C |
| 116 | da | Me3Py | N | L | Hgn | MeF | W1Me | KCOpipzaa | W1Me | T | Hgn | C |
| 117 | da | Me3Py | N | L | Hgl | MeE | W1Me | KCOpipzaa | W1Me | T | Hgn | C |
| 118 | da | Me3Py | N | Cbg | Hgn | MeN | W1Me | V | W1Me | T | Hgn | C |
| 119 | da | Me3Py | N | Cbg | Hgn | Me3Py | W1Me | V | W1Me | T | Hgn | C |
| 120 | da | MeF | N | Cbg | Hgn | MeN | W1Me | V | W1Me | T | Hgn | C |
| 121 | da | Me3Py | N | L | Hgn | MeF | W1Me | KCOpipzaa | W1Me | T | Hgn | C |
| 122 | da | Me3Py | N | L | Hgn | MeE | W1Me | KCOpipzaa | W1Me | T | Hgn | C |
| 123 | F | H | N | L | S | MeF | W1Me | Hcit | W1Me | C | | |
| 124 | df3CON | Me3Py | N | L | S | MeF | W1Me7Cl | Hcit | W1Me | C | | |
| 125 | df3CON | Me3Py | N | L | S | Me3Py | W1Me7Cl | Hcit | W1Me | C | | |
| 126 | df3CON | Me3Py | N | Cbg | S | Me3Py | W1Me7Cl | Hcit | W1Me | C | | |
| 127 | df3CON | Me3Py | N | Cbg | Qglucamine | Me3Py | W1Me7Cl | KCOpipzaa | W1Me | C | | |
| 128 | df3CON | Me3Py | N | Cbg | Qglucamine | Me3Py | W1Me7Cl | KCOpipzaa | W1Me | C | | |
| 129 | df3CON | Me4Py2 | N | Cbg | Qglucamine | Me3Py | W1Me7Cl | KCOpipzaa | W1Me | C | | |
| | | NH2 | | | | | | | | | | |
| 130 | df3CON | Me3Py | N | Cbg | Qglucamine | MeE | W1Me7Cl | KCOpipzaa | W1Me | C | | |
| 131 | df3CON | Me3Py | N | Cbg | Qglucamine | MeE | W1Me7Cl | Hcit | W1Me | C | | |
| 132 | df3CON | MeF3H | N | Cbg | Hgn | MeE | W1Me7Cl | KCOpipzaa | W1Me | C | | |
| 133 | df3CON | Me3Py | N | Cbg | Hgn | MeE | W1Me7Cl | KCOpipzaa | W1Me | CdMe | | |
| 134 | df3CON | Me3Py | N | Cbg | Hgn | MeE | W1Me7Cl | KCOpipzaa | W1Me | C3SMe | | |
| 135 | df3CON | Me3Py | N | Cbg | Hgn | MeE | W1Me7Cl | KCOpipzaa | W1Me | C3RMe | | |
| 136 | F | H | N | L | S | MeF | W1Me | Hcit | W1Me | C | | |
| 137 | F | H | N | L | S | MeF | W1Me | Hcit | W1Me | C | | |
| 138 | F | H | N | L | S | MeF | W1Me | Hcit | W1Me | C | | |
| 139 | da | MeF | N | Cba | T | N | W1Me | Hcit | W1Me | C | | |
| 140 | F | 4Py | N | L | E | MeF | W1Me | 3Py6NH2 | W1Me | C | | |
| 141 | F | H | N | L | S | MeF | W1Me | Hcit | W1Me | C | | |
| 142 | df3CON | Me3Py | N | L | S | MeF | W1Me7Cl | Hcit | W1Me | C | | |
| 143 | df3CON | Me3Py | N | L | S | Me3Py | W1Me7Cl | Hcit | W1Me | C | | |
| 144 | df3CON | Me3Py | N | Cbg | S | Me3Py | W1Me7Cl | Hcit | W1Me | C | | |
| 145 | df3CON | Me3Py | N | Cbg | Qglucamine | Me3Py | W1Me7Cl | KCOpipzaa | W1Me | C | | |
| 146 | df3CON | Me3Py | N | Cbg | Qglucamine | Me3Py | W1Me7Cl | Qglucamine | W1Me | C | | |
| 147 | df3CON | Me3Py | N | Cbg | Hgn | MeE | W1Me7Cl | KCOpipzaa | W1Me | C | | |
| 148 | df3CON | Me3Py | N | Cbg | Qglucamine | MeE | W1Me7Cl | Hcit | W1Me | C | | |
| 149 | df3CON | MeF3H | N | Cbg | Hgn | MeE | W1Me7Cl | KCOpipzaa | W1Me | C | | |
| 150 | F | 4Py2NH2 | L | Hcit | W1Me | MeF | W1Me | C | | | | |
| 151 | F | 4Py2NH2 | L | MeA | W1Me | MeF | W1Me | C | | | | |
| 152 | F | 4Py 2NH2 | L | MeHcit | W1Me | MeF | W1Me | C | | | | |
| 153 | F | 4Py2NH2 | L | Hcit | W1Me | MeF | W1Me | C | | | | |
| 154 | F | 4Py2NH2 | L | Hcit | W1Me | MeF | W1Me | C | | | | |
| 155 | F | 4Py2NH2 | L | Hcit | W1Me | MeF | W1Me | C | | | | |
| 156 | F | 4Py | L | G | W1Me | MeF | W1Me | C | | | | |
| 157 | F | 4Py2NH2 | L | Hcit | W1Me | MeF | W1Me | C | | | | |
| 158 | da | 4Py2NH2 | MeF | W1Me | MeF | W1Me | C | | | | | |
| 159 | df3CON | Me3Py | N | Cbg | Hgn | MeE | W1Me7N | KCOpipzaa | W1Me | T | Hgn | C |
| 160 | da | Me3Py | N | Cbg | Hgn | MeE | W1Me7N | KCOpipzaa | W1Me | T | Hgn | C |
| 161 | da | Me3Py | N | Cbg | K | MeE | W1Me7C1 | KCOpipzaa | W1Me | T | Hgn | C |
| 162 | df3CON | Me3Py | N | Cbg | K | MeE | W1Me7C1 | KCOpipzaa | W1Me | T | Hgn | C |
| 163 | da | Me3Py | N | Cbg | K | MeE | W1Me7C1 | KCOpipzaa | W1Me | T | Hgn | C |
| 164 | df3CON | Me3Py | N | Cbg | K | MeE | W1Me | T | Hgn | C | | |
| 165 | da | Me3Py | N | Cbg | Hgn | MeE | W1Me7C1 | KCOpipzaa | W1Me | T | K | C |
| 166 | da | Me3Py | N | Cbg | K | MeE | W1Me7C1 | KCOpipzaa | W1Me | T | Hgn | C |
| 167 | da | Me3Py | N | L | Hgn | MeE | W1Me | KCOpipzaa | W1Me | T | E | C |
| 168 | da | Me3Py | N | L | Hgn | MeE | W1Me | KCOpipzaa | W1Me | T | hArg | C |
| 169 | da | Me3Py | N | L | Hgn | MeE | W1Me | KCOpipzaa | W1Me | T | hCit | C |
| 170 | da | Me3Py | N | L | Hgn | MeN | W1Me | KCOpipzaa | W1Me | T | hArg | C |
| 171 | df3CON | Me3Py | N | Cbg | Hgn | MeE | W1Me7Cl | KCOpipzaa | W1Me | T | hArg | C |

TABLE B6-1
Example peptides in Table B6-1 include the indicated SEQ ID No: corresponding
to Table B6 and, optionally, an additional linker structure. The term
“Term” means the functional group at C-terminus of the peptide.

| | | | | | SEQ ID NO: |
|---|---|---|---|---|---|
| | | C-term | | | (including |
| Example No. | SEQ ID No: | extension | Linker | Term | linker) |
| PDC_EphA2-00001417 | 1 | | | —NH₂ | |
| PDC_EphA2-00009998 | 2 | | | —NH₂ | |
| PDC_EphA2-00009999 | 3 | | | —NH₂ | |
| PDC_EphA2-00010000 | 4 | | | —NH₂ | |
| PDC_EphA2-00010001 | 5 | | | —NH₂ | |
| PDC_EphA2-00010002 | 6 | | | —NH₂ | |
| PDC_EphA2-00010003 | 7 | | | —NH₂ | |
| PDC_EphA2-00010004 | 8 | | | —NH₂ | |
| PDC_EphA2-00010005 | 9 | | | —NH₂ | |
| PDC_EphA2-00010006 | 10 | | | —NH₂ | |
| PDC_EphA2-00010007 | 11 | | | —NH₂ | |
| PDC_EphA2-00010008 | 12 | | | —NH₂ | |
| PDC_EphA2-00010009 | 13 | | | —NH₂ | |
| PDC_EphA2-00010010 | 14 | | | —NH₂ | |
| PDC_EphA2-00010011 | 15 | | | —NH₂ | |
| PDC_EphA2-00010012 | 16 | | | —NH₂ | |
| PDC_EphA2-00013693 | 17 | | | —NH₂ | |
| PDC_EphA2-00013703 | 18 | | | —NH₂ | |
| PDC_EphA2-00013704 | 19 | | | —NH₂ | |
| PDC_EphA2-00013705 | 20 | | | —NH₂ | |
| PDC_EphA2-00013917 | 21 | | | —NH₂ | |
| PDC_EphA2-00018526 | 22 | | | —NH₂ | |
| PDC_EphA2-00018527 | 23 | | | —NH₂ | |
| PDC_EphA2-00018528 | 24 | | | —NH₂ | |
| PDC_EphA2-00018529 | 25 | | | —NH₂ | |
| PDC_EphA2-00018530 | 26 | | | —NH₂ | |
| PDC_EphA2-00018531 | 27 | | | —NH₂ | |
| PDC_EphA2-00018532 | 28 | | | —NH₂ | |
| PDC_EphA2-00018533 | 29 | | | —NH₂ | |
| PDC_EphA2-00018534 | 30 | | | —NH₂ | |
| PDC_EphA2-00018535 | 31 | | | —NH₂ | |
| PDC_EphA2-00018536 | 32 | | | —NH₂ | |
| PDC_EphA2-00018537 | 33 | | | —NH₂ | |
| PDC_EphA2-00018538 | 34 | | | —NH₂ | |
| PDC_EphA2-00018539 | 35 | | | —NH₂ | |
| PDC_EphA2-00018540 | 36 | | | —NH₂ | |
| PDC_EphA2-00019421 | 37 | | | —NH₂ | |
| PDC_EphA2-00019422 | 38 | | | —NH₂ | |
| PDC_EphA2-00019423 | 39 | | | —NH₂ | |
| PDC_EphA2-00019424 | 40 | | | —NH₂ | |
| PDC_EphA2-00019425 | 41 | | | —NH₂ | |
| PDC_EphA2-00019426 | 42 | | | —NH₂ | |
| PDC_EphA2-00019427 | 43 | | | —NH₂ | |
| PDC_EphA2-00019428 | 44 | | | —NH₂ | |
| PDC_EphA2-00019429 | 45 | | | —NH₂ | |
| PDC_EphA2-00019430 | 46 | | | —NH₂ | |
| PDC_EphA2-00019431 | 47 | | | —NH₂ | |
| PDC_EphA2-00019432 | 48 | | | —NH₂ | |
| PDC_EphA2-00019433 | 49 | | | —NH₂ | |
| PDC_EphA2-00019434 | 50 | | | —NH₂ | |
| PDC_EphA2-00019435 | 51 | | | —NH₂ | |
| PDC_EphA2-00019436 | 52 | | | —NH₂ | |
| PDC_EphA2-00019437 | 53 | | | —NH₂ | |
| PDC_EphA2-00019438 | 54 | | | —NH₂ | |
| PDC_EphA2-00019439 | 55 | | | —NH₂ | |
| PDC_EphA2-00019440 | 56 | | | —NH₂ | |
| PDC_EphA2-00019441 | 57 | | | —NH₂ | |
| PDC_EphA2-00019442 | 58 | | | —NH₂ | |
| PDC_EphA2-00026424 | 59 | | | —NH₂ | |
| PDC_EphA2-00026425 | 60 | | | —NH₂ | |
| PDC_EphA2-00026426 | 61 | | | —NH₂ | |
| PDC_EphA2-00026521 | 62 | | | —NH₂ | |
| PDC_EphA2-00026522 | 63 | | | —NH₂ | |
| PDC_EphA2-00026523 | 64 | | | —NH₂ | |
| PDC_EphA2-00026524 | 65 | | | —NH₂ | |
| PDC_EphA2-00026525 | 66 | | | —NH₂ | |
| PDC_EphA2-00026526 | 67 | | | —NH₂ | |
| PDC_EphA2-00026527 | 68 | | | —NH₂ | |
| PDC_EphA2-00026528 | 69 | | | —NH₂ | |
| PDC_EphA2-00026529 | 70 | | | —NH₂ | |
| PDC_EphA2-00010011-C003 | 71 | | bA dk | —NH₂ | 180 |
| PDC_EphA2-00010012-C003 | 72 | | bA dk | —NH₂ | 181 |
| PDC_EphA2-00013693-C003 | 73 | | bA dk | —NH₂ | 182 |
| PDC_EphA2-00008082 | 74 | | bA dkAc | —NH₂ | 183 |
| PDC_EphA2-00008083 | 75 | | bA dkAc | —NH₂ | 184 |
| PDC_EphA2-00008084 | 76 | | bA dkAc | —NH₂ | 185 |
| PDC_EphA2-00008085 | 77 | | bA dkAc | —NH₂ | 186 |
| PDC_EphA2-00008086 | 78 | | bA dkAc | —NH₂ | 187 |
| PDC_EphA2-00008087 | 79 | | bA dkAc | —NH₂ | 188 |
| PDC_EphA2-00008088 | 80 | | bA dkAc | —NH₂ | 189 |
| PDC_EphA2-00008089 | 81 | | KAc | —NH₂ | 190 |
| PDC_EphA2-00008090 | 82 | | dkAc | —NH₂ | 191 |
| PDC_EphA2-00008091 | 83 | | bA dkAc | —NH₂ | 192 |
| PDC_EphA2-00008092 | 84 | | bA dkAc | —NH₂ | 193 |
| PDC_EphA2-00008093 | 85 | | bA dkAc | —NH₂ | 194 |
| PDC_EphA2-00008094 | 86 | | bA dkAc | —NH₂ | 195 |
| PDC_EphA2-00008095 | 87 | | bA dkAc | —NH₂ | 196 |
| PDC_EphA2-00007196 | 88 | | bA dkAc | —NH₂ | 197 |
| PDC_EphA2-00008097 | 89 | | bA dkAc | —NH₂ | 198 |
| PDC_EphA2-00008093-C002 | 90 | | dk | —NH₂ | 199 |
| PDC_EphA2-00007196-C002 | 91 | | dk | —NH₂ | 200 |
| PDC_EphA2-00010011-C002 | 92 | | dk | —NH₂ | 201 |
| PDC_EphA2-00010012-C002 | 93 | | dk | —NH₂ | 202 |
| PDC_EphA2-00013693-C002 | 94 | | dk | —NH₂ | 203 |
| PDC_EphA2-00019437-C002 | 95 | | dk | —NH₂ | 204 |
| PDC_EphA2-00022593-C002 | 96 | | dk | —NH₂ | 205 |
| PDC_EphA2-00022594-C002 | 97 | | dk | —NH₂ | 206 |
| PDC_EphA2-00022595-C002 | 98 | | dk | —NH₂ | 207 |
| PDC_EphA2-00019440-C002 | 99 | | dk | —NH₂ | 208 |
| PDC_EphA2-00022612-C002 | 100 | | dk | —NH₂ | 209 |
| PDC_EphA2-00022613-C002 | 101 | | dk | —NH₂ | 210 |
| PDC_EphA2-00022614-C002 | 102 | | dk | —NH₂ | 211 |
| PDC_EphA2-00022615-C002 | 103 | | dk | —NH₂ | 212 |
| PDC_EphA2-00013703-C002 | 104 | | dk | —NH₂ | 213 |
| PDC_EphA2-00013704-C002 | 105 | | dk | —NH₂ | 214 |
| PDC_EphA2-00013705-C002 | 106 | | dk | —NH₂ | 215 |
| PDC_EphA2-00026424-C002 | 107 | | dk | —NH₂ | 216 |
| PDC_EphA2-00026425-C002 | 108 | | dk | —NH₂ | 217 |
| PDC_EphA2-00026426-C002 | 109 | | dk | —NH₂ | 218 |
| PDC_EphA2-00026624 | 110 | | dk | —OH | 219 |
| PDC_EphA2-00026625 | 111 | | dk | —OH | 220 |
| PDC_EphA2-00026626 | 112 | | dk | —OH | 221 |
| PDC_EphA2-00026627 | 113 | | dk | —OH | 222 |
| PDC_EphA2-00001417-C004 | 114 | | bA | —NH₂ | 223 |
| | | | dk(PEG8c- | | |
| | | | PEG2c) | | |
| PDC_EphA2-00007196-C004 | 115 | | bA | —NH₂ | 224 |
| | | | dk(PEG8c- | | |
| | | | PEG2c) | | |
| PDC_EphA2-00008093-C004 | 116 | | bA | —NH₂ | 225 |
| | | | dk(PEG8c- | | |
| | | | PEG2c) | | |
| PDC_EphA2-00008094-C004 | 117 | | bA | —NH₂ | 226 |
| | | | dk(PEG8c- | | |
| | | | PEG2c) | | |
| PDC_EphA2-00010011-C004 | 118 | | bA | —NH₂ | 227 |
| | | | dk(PEG8c- | | |
| | | | PEG2c) | | |
| PDC_EphA2-00010012-C004 | 119 | | bA | —NH₂ | 228 |
| | | | dk(PEG8c- | | |
| | | | PEG2c) | | |
| PDC_EphA2-00013693-C004 | 120 | | bA | —NH₂ | 229 |
| | | | dk(PEG8c- | | |
| | | | PEG2c) | | |
| PDC_EphA2-00008093-C010 | 121 | | dk(bA- | —NH₂ | 230 |
| | | | MeG- | | |
| | | | MeG- | | |
| | | | MeG- | | |
| | | | MeG- | | |
| | | | MeG- | | |
| | | | MeG- | | |
| | | | MeG- | | |
| | | | MeG- | | |
| | | | MeG- | | |
| | | | MeG) | | |
| | | | (SEQ ID | | |
| | | | NO: 279) | | |
| PDC_EphA2-00007196-C010 | 122 | | dk(bA- | —NH₂ | 231 |
| | | | MeG- | | |
| | | | MeG- | | |
| | | | MeG- | | |
| | | | MeG- | | |
| | | | MeG- | | |
| | | | MeG- | | |
| | | | MeG- | | |
| | | | MeG- | | |
| | | | MeG- | | |
| | | | MeG) | | |
| | | | (SEQ ID | | |
| | | | NO: 279) | | |
| PDC_EphA2-00022417 | 123 | | | —NH₂ | |
| PDC_EphA2-00026600 | 124 | | | —NH₂ | |
| PDC_EphA2-00026601 | 125 | | | —NH₂ | |
| PDC_EphA2-00026602 | 126 | | | —NH₂ | |
| PDC_EphA2-00026603 | 127 | | | —NH₂ | |
| PDC_EphA2-00026604 | 128 | | | —NH₂ | |
| PDC_EphA2-00026605 | 129 | | | —NH₂ | |
| PDC_EphA2-00026606 | 130 | | | —NH₂ | |
| PDC_EphA2-00026607 | 131 | | | —NH₂ | |
| PDC_EphA2-00026608 | 132 | | | —NH₂ | |
| PDC_EphA2-00026609 | 133 | | | —NH₂ | |
| PDC_EphA2-00026610 | 134 | | | —NH₂ | |
| PDC_EphA2-00026611 | 135 | | | —NH₂ | |
| PDC_EphA2-00022606 | 136 | | G | —NH₂ | 232 |
| PDC_EphA2-00022607 | 137 | | KAc | —NH₂ | 233 |
| PDC_EphA2-00022608 | 138 | | dkAc | —NH₂ | 234 |
| PDC_EphA2-00021407-L092 | 139 | | G | —NH₂ | 235 |
| PDC_EphA2-00022057-L092 | 140 | | G | —NH₂ | 236 |
| PDC_EphA2-00022417-C002 | 141 | | dk | —NH₂ | 237 |
| PDC_EphA2-00026600-C001 | 142 | | K | —NH₂ | 238 |
| PDC_EphA2-00026601-C001 | 143 | | K | —NH₂ | 239 |
| PDC_EphA2-00026602-C001 | 144 | | K | —NH₂ | 240 |
| PDC_EphA2-00026603-C001 | 145 | | K | —NH₂ | 241 |
| PDC_EphA2-00026604-C001 | 146 | | K | —NH₂ | 242 |
| PDC_EphA2-00026606-C001 | 147 | | K | —NH₂ | 243 |
| PDC_EphA2-00026607-C001 | 148 | | K | —NH₂ | 244 |
| PDC_EphA2-00026608-C001 | 149 | | K | —NH₂ | 245 |
| PDC_EphA2-00020708 | 150 | | | —NH₂ | |
| PDC_EphA2-00026479 | 151 | | | —NH₂ | |
| PDC_EphA2-00026481 | 152 | | | —NH₂ | |
| PDC_EphA2-00022601 | 153 | | G | —NH₂ | 246 |
| PDC_EphA2-00022602 | 154 | | KAc | —NH₂ | 247 |
| PDC_EphA2-00022603 | 155 | | dkAc | —NH₂ | 248 |
| PDC_EphA2-00020295-L092 | 156 | | G | —NH₂ | 249 |
| PDC_EphA2-00020708-C002 | 157 | | dk | —NH₂ | 250 |
| PDC_EphA2-00019498-L092 | 158 | | G | —NH₂ | 251 |
| PDC_EphA2-00027094-C002 | 159 | | dk | —NH₂ | 252 |
| PDC_EphA2-00027095-C002 | 160 | | dk | —NH₂ | 253 |
| PDC_EphA2-00027090 | 161 | de | | —OH | 254 |
| PDC_EphA2-00027091 | 162 | de | | —OH | 255 |
| PDC_EphA2-00027092 | 163 | de | | —NH₂ | 256 |
| PDC_EphA2-00027106 | 162 | de | | —NH₂ | 257 |
| PDC_EphA2-00007196-C008 | 88 | | dk df | —NH₂ | 258 |
| PDC_EphA2-00007196-C009 | 88 | | dk(G-df) | —NH₂ | 259 |
| PDC_EphA2-00027131 | 165 | | tma | —NH₂ | 260 |
| PDC_EphA2-00027129 | 165 | | df | —NH₂ | 261 |
| PDC_EphA2-00027128 | 165 | | de | —NH₂ | 262 |
| PDC_EphA2-00027130 | 166 | de | tma | —NH₂ | 263 |
| PDC_EphA2-00027127 | 166 | de | df | —NH₂ | 264 |
| PDC_EphA2-00027126 | 166 | de | de | —NH₂ | 265 |
| PDC_EphA2-00008010-C002 | 168 | | dk | —NH₂ | 267 |
| PDC_EphA2-00007199-C002 | 167 | | dk | —NH₂ | 267 |
| PDC_EphA2-00007180-C002 | 169 | | dk | —NH₂ | 268 |
| PDC_EphA2-00007198-C002 | 170 | | dk | —NH₂ | 269 |
| PDC_EphA2-00019443-C002 | 171 | | dk | —NH₂ | 270 |
| PDC_EphA2-00019444-C002 | 171 | | dk | —OH | 271 |

### Example B7: PDC_EphA2-00026626 (SEQ ID No: 221)

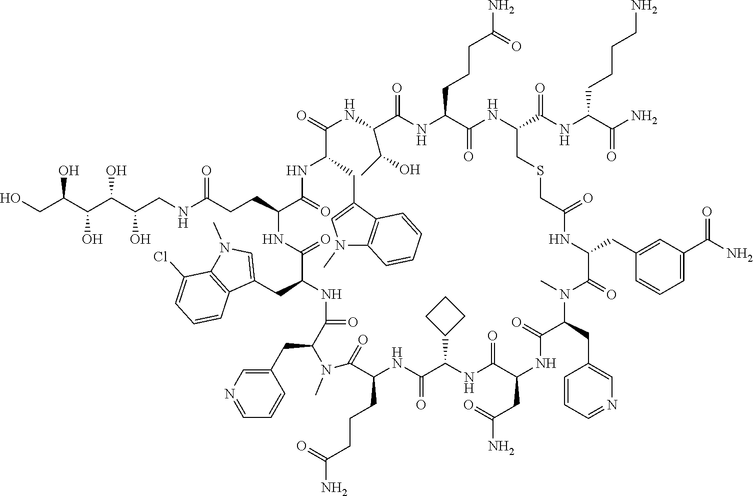 [SMILES: C[C@@H](O)[C@@H]1NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3c(Cl)cccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(=O)N[C@H](CCCCN)C(N)=O)NC(=O)[C@H](CCCC(N)=O)NC1=O]

The peptide sequence was synthesized on Fmoc-dk (Boc)-Alko Resin (0.7 mmol/g, 0.250 mmol) following general peptide synthesis method A. The obtained peptide on the resin was subjected to the general method ClAc-4. The polypeptide on the resin was treated with Cleavage Cocktail-A (15 mL) to furnish the linear peptide. The crude containing the linear peptide (0.250 mmol as theoretical based on the resin used) was subjected to the peptide cyclization condition-2. The resulting residue was purified by preparative reversed-phase HPLC (Method P-A). Pure fractions were combined and lyophilized to afford the title macrocyclic peptide. HPLC-MS (Method A-A): 5.32 min, ESI-MS m/z: 1071.8 [M+2H]2+, AUC (UV 225 nm): 93%.

The following Examples were prepared by following similar methods described in the examples above. The amino acid sequence for each peptide was shown in Table B6 and B6-1.

TABLE B7

| Example | | |
|---|---|---|
| No. | Structure | Analytics |
| PDC_EphA2- 00001417 (SEQ ID NO: 1) | 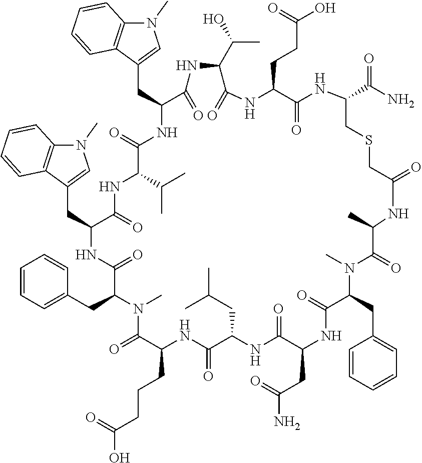 [SMILES: CC(C)C[C@@H]1NC(=O)[C@H](CC(N)=O)NC(=O)[C@H](Cc2ccccc2)N(C)C(=O)[C@@H](C)NC(=O)CSC[C@@H](C(N)=O)NC(=O)[C@H](CCC(=O)O)NC(=O)[C@H]([C@@H](C)O)NC(=O)[C@H](Cc2cn(C)c3ccccc23)NC(=O)[C@H](C(C)C)NC(=O)[C@H](Cc2cn(C)c3ccccc23)NC(=O)[C@H](Cc2ccccc2)N(C)C(=O)[C@H](CCCC(=O)O)NC1=O] | HPLC-MS (Method A-C): 3.56 min ESI-MS m/z: 827.7 [M + 2H]2+ |
| PDC_EphA2- 00008082 (SEQ ID NO: 183) | 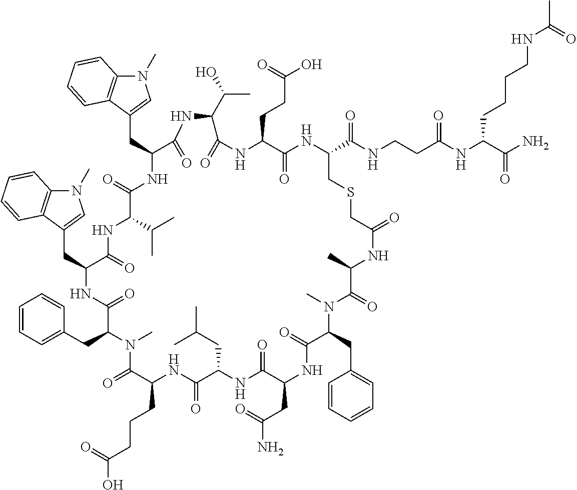 [SMILES: CC(=O)NCCCC[C@@H](NC(=O)CCNC(=O)[C@@H]1CSCC(=O)N[C@H](C)C(=O)N(C)[C@@H](Cc2ccccc2)C(=O)N[C@@H](CC(N)=O)C(=O)N[C@@H](CC(C)C)C(=O)N[C@@H](CCCC(=O)O)C(=O)N(C)[C@@H](Cc2ccccc2)C(=O)N[C@@H](Cc2cn(C)c3ccccc23)C(=O)N[C@@H](C(C)C)C(=O)N[C@@H](Cc2cn(C)c3ccccc23)C(=O)N[C@@H]([C@@H](C)O)C(=O)N[C@@H](CCC(=O)O)C(=O)N1)C(N)=O] | HPLC-MS (Method A-E): 3.14 min ESI-MS m/z: 948.4 [M + 2H]2+ |
| PDC_EphA2- 00008083 (SEQ ID NO: 184) | 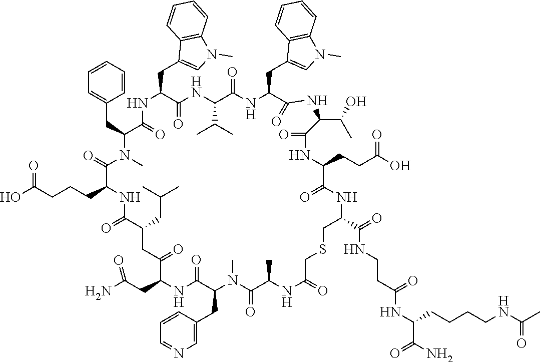 | HPLC-MS (Method A-B): 4.98 min ESI-MS m/z: 949.2 [M + 2H]2+ |
| PDC_EphA2- 00008084 (SEQ ID NO: 185) | 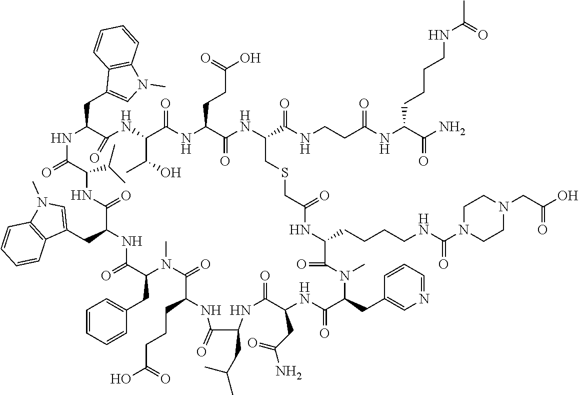 | HPLC-MS (Method A-B): 4.64 min ESI-MS m/z: 1062.8 [M + 2H]2+ |
| PDC_EphA2- 00008085 (SEQ ID NO: 186) | 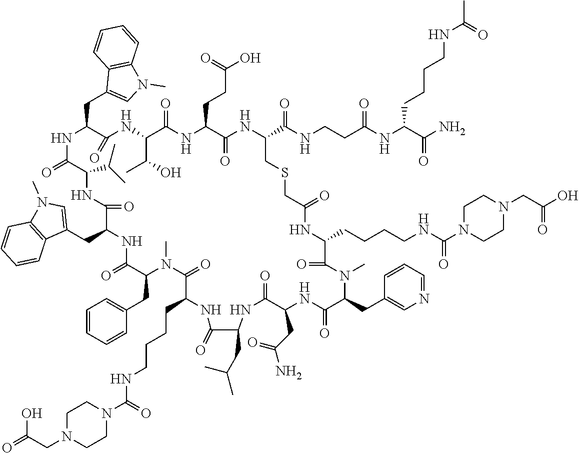 | HPLC-MS (Method A-B): 4.16 min ESI-MS m/z: 1140.3 [M + 2H]2+ |
| PDC_EphA2- 00008086 (SEQ ID NO: 187) | 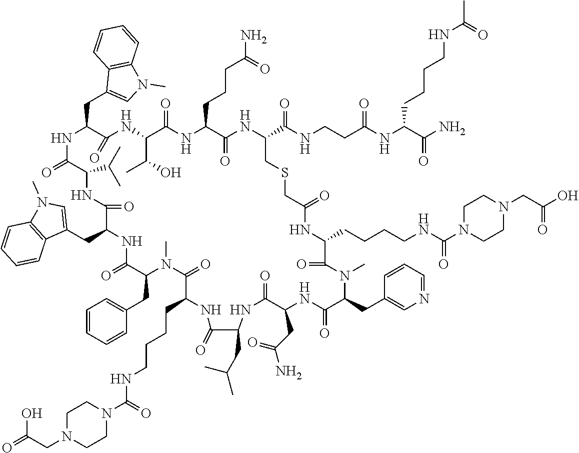 [SMILES: CC(=O)NCCCC[C@@H](NC(=O)CCNC(=O)[C@@H]1CSCC(=O)N[C@H](CCCCNC(=O)N2CCN(CC(=O)O)CC2)C(=O)N(C)[C@@H](Cc2cccnc2)C(=O)N[C@@H](CC(N)=O)C(=O)N[C@@H](CC(C)C)C(=O)N[C@@H](CCCCNC(=O)N2CCN(CC(=O)O)CC2)C(=O)N(C)[C@@H](Cc2ccccc2)C(=O)N[C@@H](Cc2cn(C)c3ccccc23)C(=O)N[C@@H](C(C)C)C(=O)N[C@@H](Cc2cn(C)c3ccccc23)C(=O)N[C@@H]([C@@H](C)O)C(=O)N[C@@H](CCCC(N)=O)C(=O)N1)C(N)=O] | HPLC-MS (Method A-B): 4.43 min ESI-MS m/z: 1146.7 [M + 2H]2+ |
| PDC_EphA2- 00008087 (SEQ ID NO: 188) | 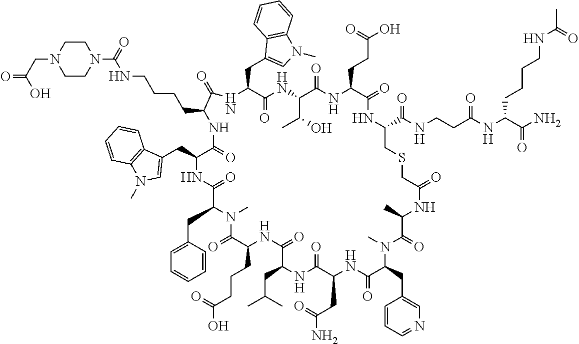 [SMILES: CC(=O)NCCCC[C@@H](NC(=O)CCNC(=O)[C@@H]1CSCC(=O)N[C@H](C)C(=O)N(C)[C@@H](Cc2cccnc2)C(=O)N[C@@H](CC(N)=O)C(=O)N[C@@H](CC(C)C)C(=O)N[C@@H](CCCC(=O)O)C(=O)N(C)[C@@H](Cc2ccccc2)C(=O)N[C@@H](Cc2cn(C)c3ccccc23)C(=O)N[C@@H](CCCCNC(=O)N2CCN(CC(=O)O)CC2)C(=O)N[C@@H](Cc2cn(C)c3ccccc23)C(=O)N[C@@H]([C@@H](C)O)C(=O)N[C@@H](CCC(=O)O)C(=O)N1)C(N)=O] | HPLC-MS (Method A-B): 4.33 min ESI-MS m/z: 1048.6 [M + 2H]2+ |
| PDC_EphA2- 00008088 (SEQ ID NO: 189) | 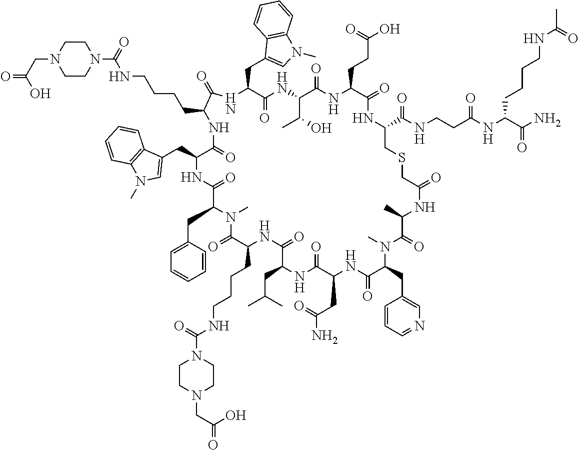 | HPLC-MS (Method A-B): 3.87 min ESI-MS m/z: 751.2 [M + 3H]3+ |
| PDC_EphA2- 00008089 (SEQ ID NO: 190) | 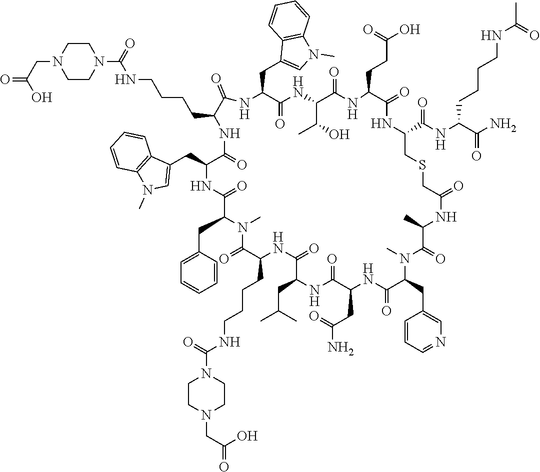 | HPLC-MS (Method A-B): 3.45 min ESI-MS m/z: 757.5 [M + 3H]3+ |
| PDC_EphA2- 00008090 (SEQ ID NO: 191) | 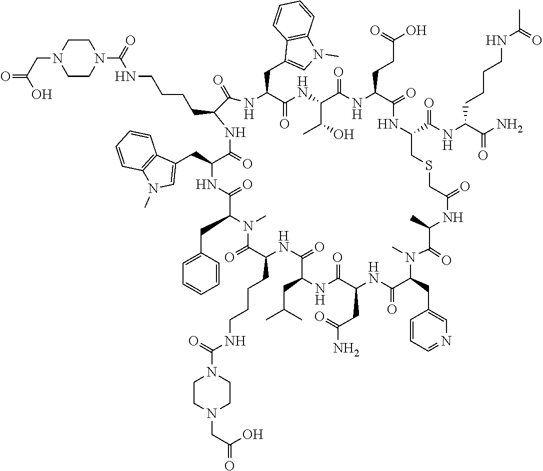 | HPLC-MS (Method A-B): 3.91 min ESI-MS m/z: 727.5 [M + 3H]3+ |
| PDC_EphA2- 00008091 (SEQ ID NO: 192) | 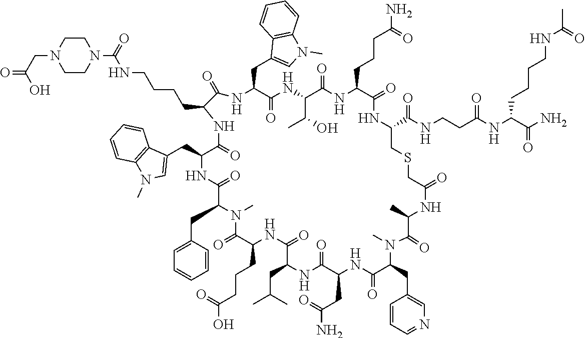 | HPLC-MS (Method A-B): 3.91 min ESI-MS m/z: 704.0 [M + 3H]3+ |
| PDC_EphA2- 00008092 (SEQ ID NO: 193) | 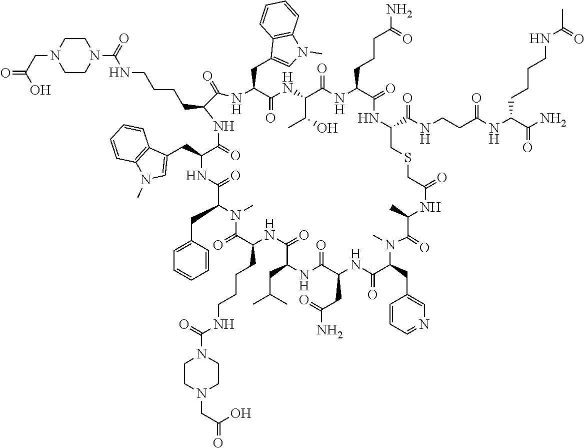 | HPLC-MS (Method A-B): 3.42 min ESI-MS m/z: 755.6 [M + 3H]3+ |
| PDC_EphA2- 00008093 (SEQ ID NO: 194) | 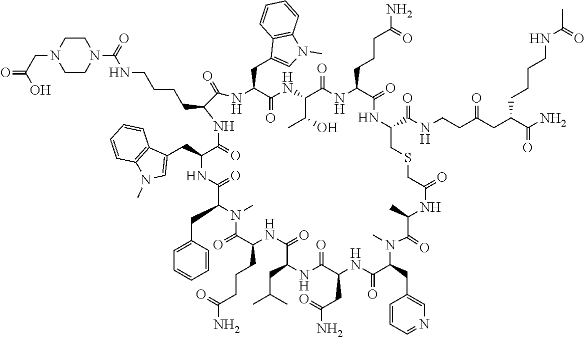 | HPLC-MS (Method A-B): 3.72 min ESI-MS m/z: 703.6 [M + 3H]3+ |
| PDC_EphA2- 00008094 (SEQ ID NO: 195) | 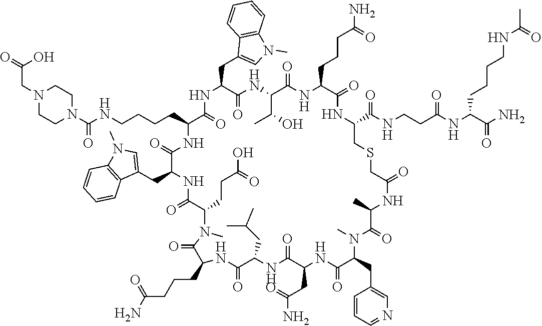 | HPLC-MS (Method A-B): 3.11 min ESI-MS m/z: 697.9 [M + 3H]3+ |
| PDC_EphA2- 00008095 (SEQ ID NO: 196) | 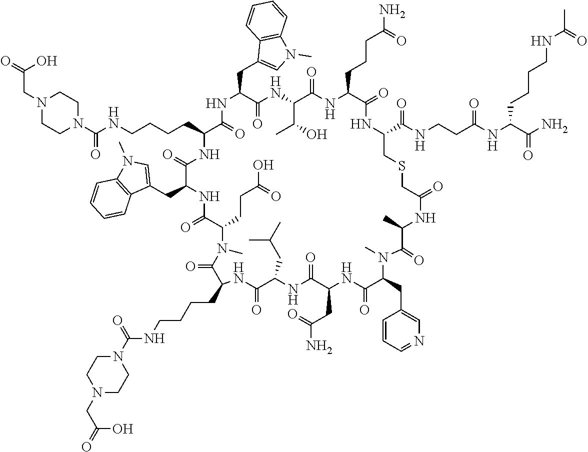 | HPLC-MS (Method A-A): 5.43 min ESI-MS m/z: 749.6 [M + 3H]3+ |
| PDC_EphA2- 00007196 (SEQ ID NO: 197) | 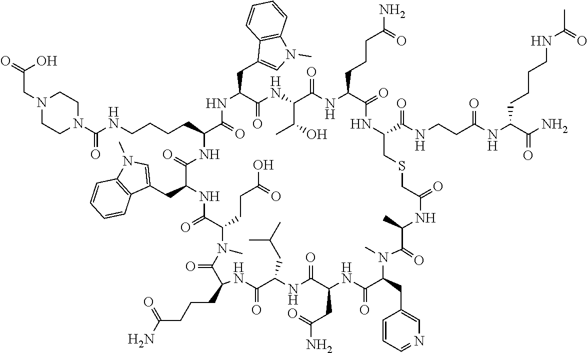 | HPLC-MS (Method A-A): 5.66 min ESI-MS m/z: 697.6 [M + 3H]3+ |
| PDC_EphA2- 00008097 (SEQ ID NO: 198) | 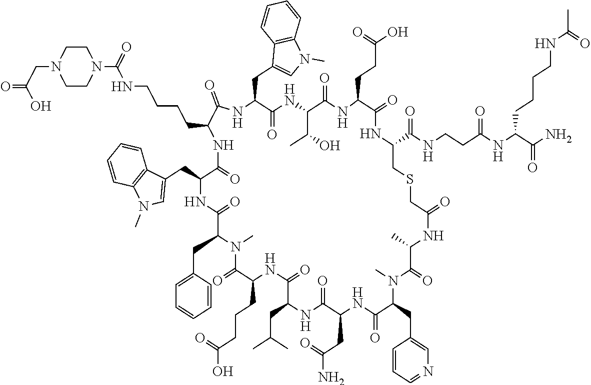 | HPLC-MS (Method A-A): 3.96 min ESI-MS m/z: 699.7 [M + 3H]3+ |
| PDC_EphA2- 00009998 (SEQ ID NO: 2) | 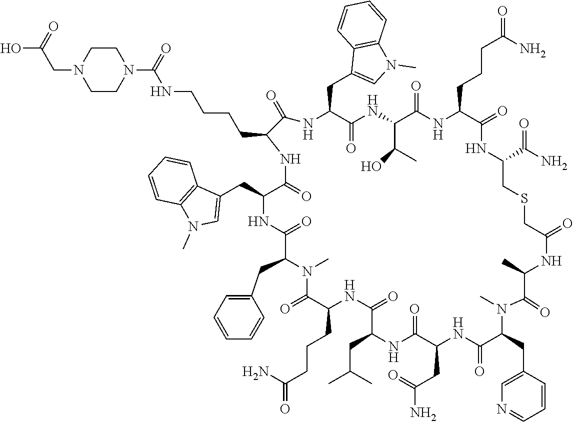 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(N)=O)NC1=O] | HPLC-MS (Method A-B): 3.82 min ESI-MS m/z: 934.0 [M + 2H]2+ |
| PDC_EphA2- 00009999 (SEQ ID NO: 3) | 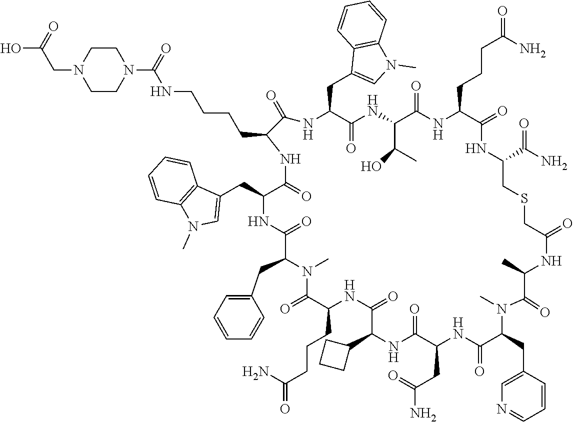 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C1=O] | HPLC-MS (Method A-B): 3.72 min ESI-MS m/z: 933.0 [M + 2H]2+ |
| PDC_EphA2- 00010000 (SEQ ID NO: 4) | 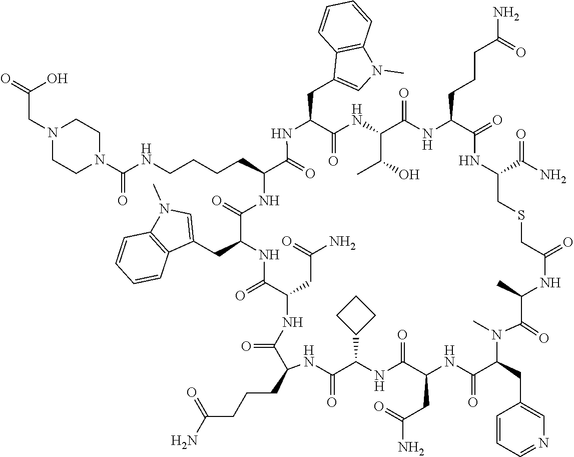 | HPLC-MS (Method A-A): 5.02 min ESI-MS m/z: 909.7 [M + 2H]2+ |
| PDC_EphA2- 00010001 (SEQ ID NO: 5) | 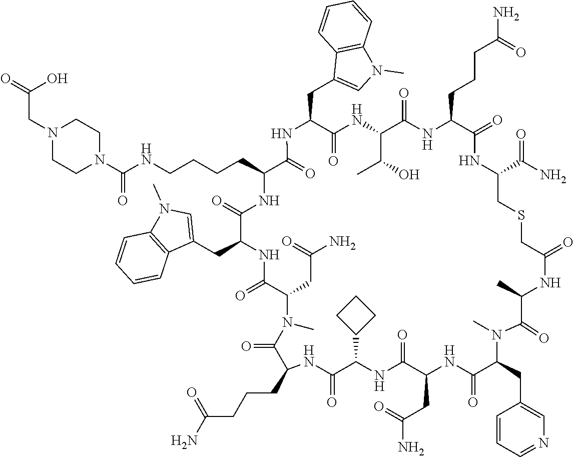 | HPLC-MS (Method A-A): 5.20 min ESI-MS m/z: 916.7 [M + 2H]2+ |
| PDC_EphA2- 00010002 (SEQ ID NO: 6) | 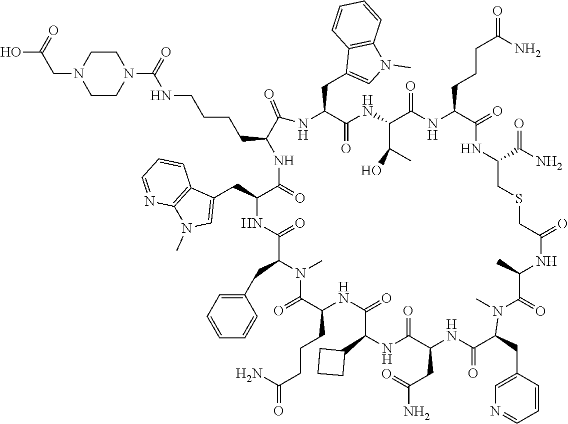 | HPLC-MS (Method A-A): 5.16 min ESI-MS m/z: 933.8 [M + 2H]2+ |
| PDC_EphA2- 00010003 (SEQ ID NO: 7) | 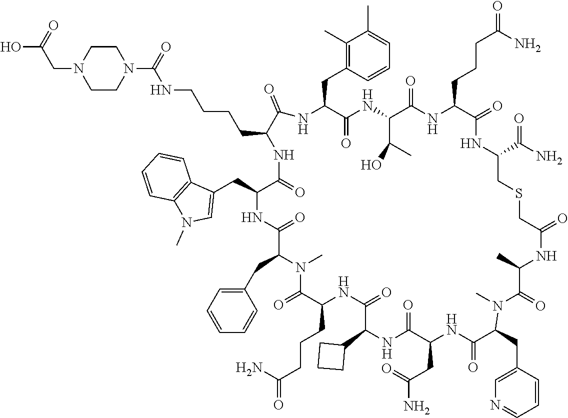 [SMILES: Cc1cccc(C[C@@H]2NC(=O)[C@H](CCCCNC(=O)N3CCN(CC(=O)O)CC3)NC(=O)[C@H](Cc3cn(C)c4ccccc34)NC(=O)[C@H](Cc3ccccc3)N(C)C(=O)[C@H](CCCC(N)=O)NC(=O)[C@H](C3CCC3)NC(=O)[C@H](CC(N)=O)NC(=O)[C@H](Cc3cccnc3)N(C)C(=O)[C@@H](C)NC(=O)CSC[C@@H](C(N)=O)NC(=O)[C@H](CCCC(N)=O)NC(=O)[C@H]([C@@H](C)O)NC2=O)c1C] | HPLC-MS (Method A-B): 3.75 min ESI-MS m/z: 920.5 [M + 2H]2+ |
| PDC_EphA2- 00010004 (SEQ ID NO: 8) | 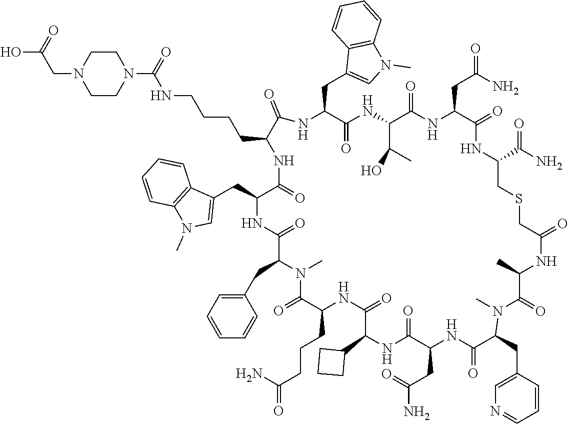 [SMILES: C[C@H]1NC(=O)CSC[C@@H](C(N)=O)NC(=O)[C@H](CC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C1=O] | HPLC-MS (Method A-B): 3.71 min ESI-MS m/z: 919.0 [M + 2H]2+ |
| PDC_EphA2- 00010005 (SEQ ID NO: 9) | 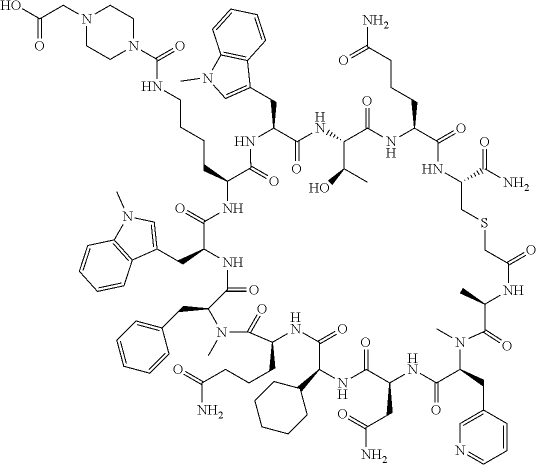 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(N)=O)NC(=O)[C@H](C2CCCCC2)NC(=O)[C@H](CC(N)=O)NC(=O)[C@H](Cc2cccnc2)N(C)C1=O] | HPLC-MS (Method A-B): 4.09 min ESI-MS m/z: 947.0 [M + 2H]2+ |
| PDC_EphA2- 00010006 (SEQ ID NO: 10) | 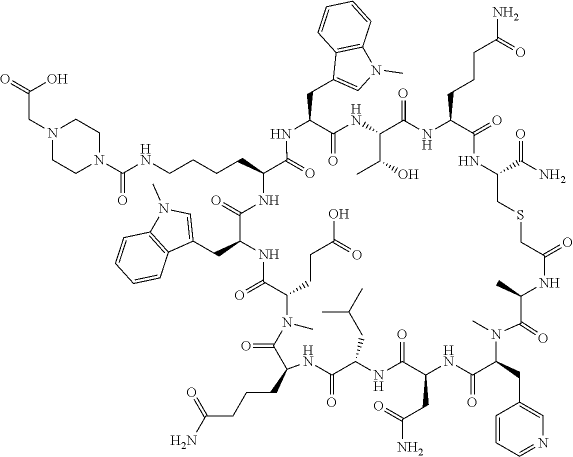 | HPLC-MS (Method A-B): 3.08 min ESI-MS m/z: 925.4 [M + 2H]2+ |
| PDC_EphA2- 00010007 (SEQ ID NO: 11) | 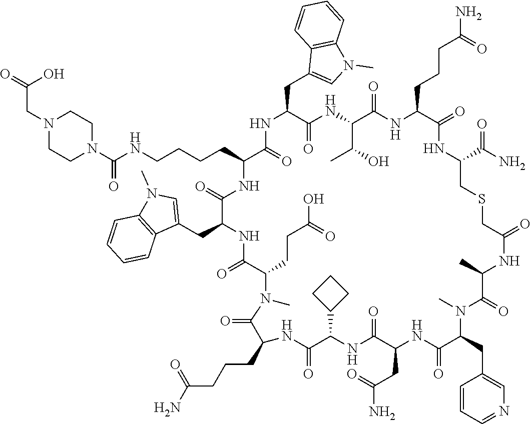 | HPLC-MS (Method A-B): 2.94 min ESI-MS m/z: 924.4 [M + 2H]2+ |
| PDC_EphA2- 00010008 (SEQ ID NO: 12) | 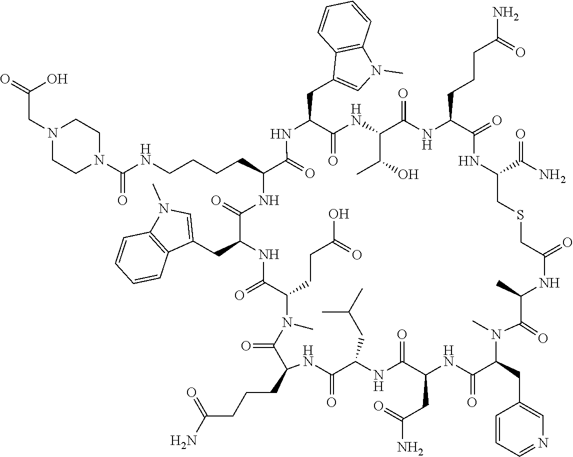 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(N)=O)NC1=O] | HPLC-MS (Method A-B): 2.95 min ESI-MS m/z: 924.9 [M + 2H]2+ |
| PDC_EphA2- 00010009 (SEQ ID NO: 13) | 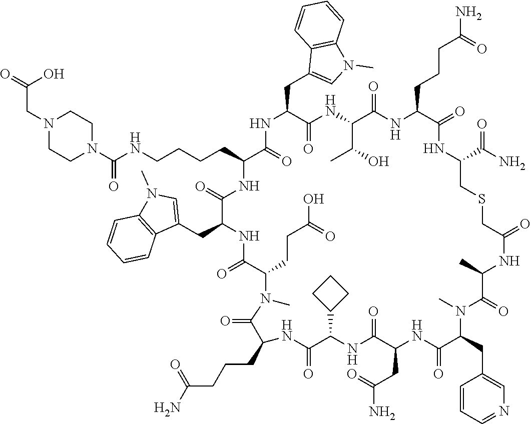 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(N)=O)NC(=O)[C@H](C2CCC2)NC(=O)[C@H](CC(N)=O)NC(=O)[C@H](Cc2cccnc2)N(C)C1=O] | HPLC-MS (Method A-A): 5.54 min ESI-MS m/z: 923.9 [M + 2H]2+ |
| PDC_EphA2- 00010010 (SEQ ID NO: 14) | 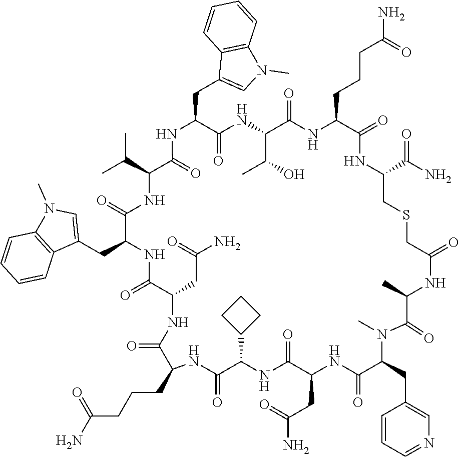 | HPLC-MS (Method A-A): 3.28 min ESI-MS m/z: 810.1 [M + 2H]2+ |
| PDC_EphA2- 00010011 (SEQ ID NO: 15) | 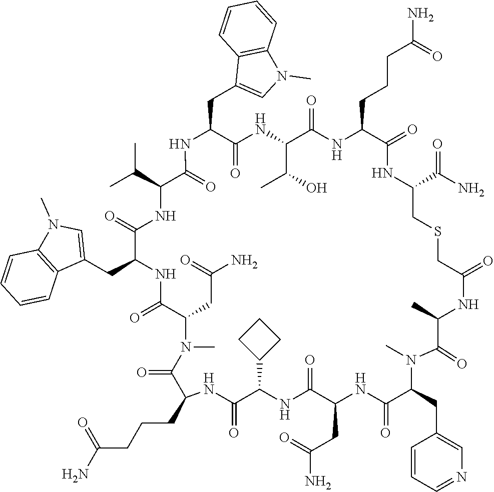 | HPLC-MS (Method A-B): 3.24 min ESI-MS m/z: 817.2 [M + 2H]2+ |
| PDC_EphA2- 00010012 (SEQ ID NO: 16) | 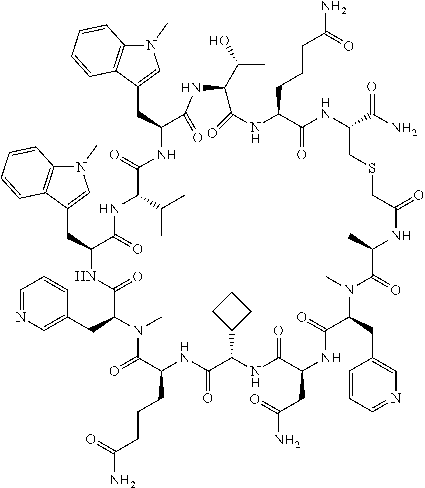 [SMILES: CC(C)[C@@H]1NC(=O)[C@H](Cc2cn(C)c3ccccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-A): 5.56 min ESI-MS m/z: 834.1 [M + 2H]2+ |
| PDC_EphA2- 00013693 (SEQ ID NO: 17) | 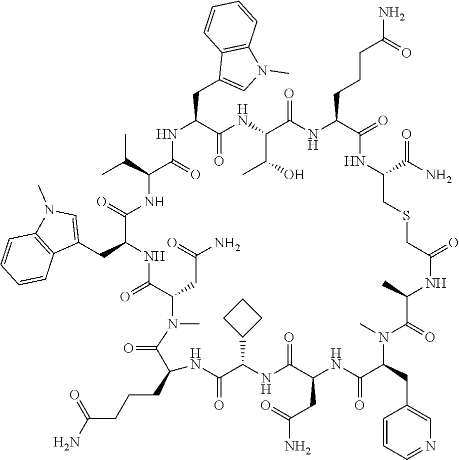 [SMILES: CC(C)[C@@H]1NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-B): 5.34 min ESI-MS m/z: 816.4 [M + 2H]2+ |
| PDC_EphA2- 00013703 (SEQ ID NO: 18) | 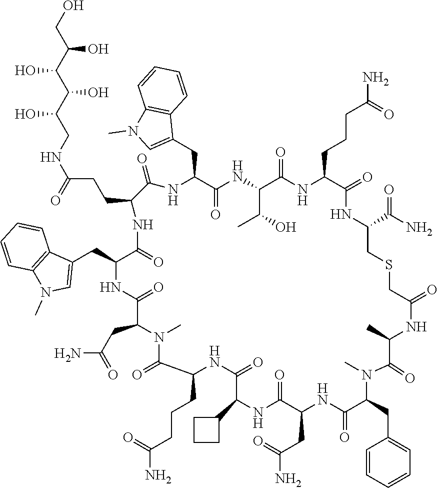 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C1=O] | HPLC-MS (Method A-B): 4.66 min ESI-MS m/z: 912.7 [M + 2H]2+ |
| PDC_EphA2- 00013704 (SEQ ID NO: 19) | 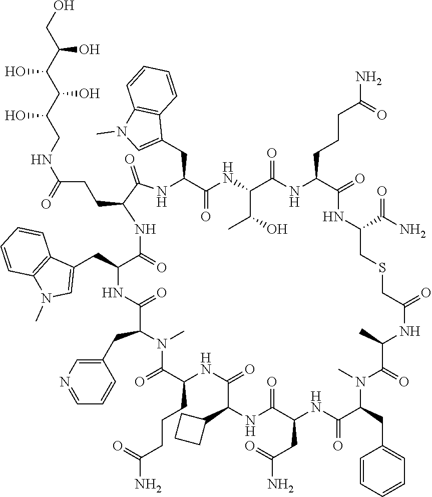 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3ccccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2ccccc2)N(C)C1=O] | HPLC-MS (Method A-B): 4.08 min ESI-MS m/z: 929.9 [M + 2H]2+ |
| PDC_EphA2- 00013705 (SEQ ID NO: 20) | 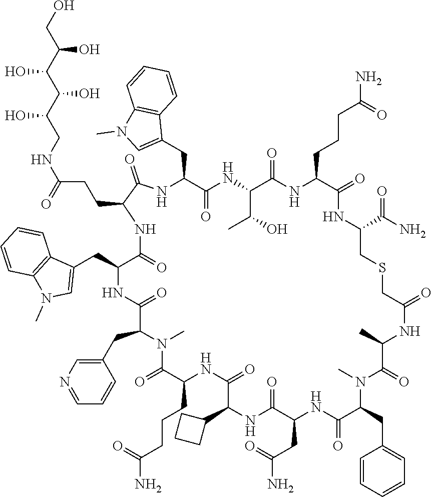 | HPLC-MS (Method A-A): 5.04 min ESI-MS m/z: 930.4 [M + 2H]2+ |
| PDC_EphA2- 00013917 (SEQ ID NO: 21) | 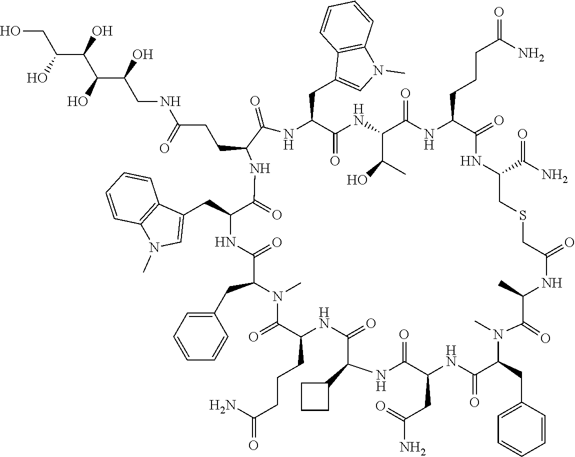 [SMILES: C[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3ccccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2ccccc2)N(C)C1=O] | HPLC-MS (Method A-B): 5.46 min ESI-MS m/z: 929.3 [M + 2H]2+ |
| PDC_EphA2- 00018526 (SEQ ID NO: 22) | 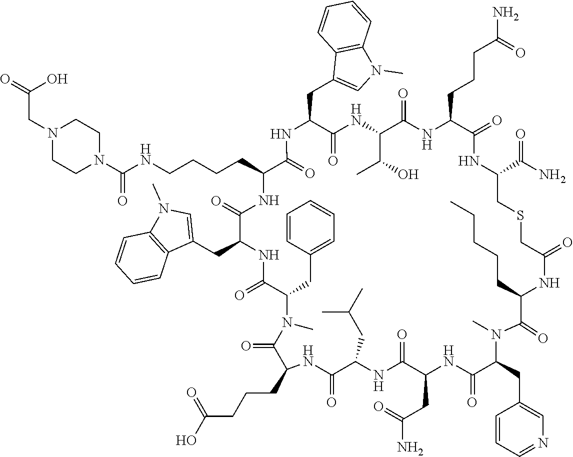 | HPLC-MS (Method A-B): 4.39 min ESI-MS m/z: 962.8 [M + 2H]2+ |
| PDC_EphA2- 00018527 (SEQ ID NO: 23) | 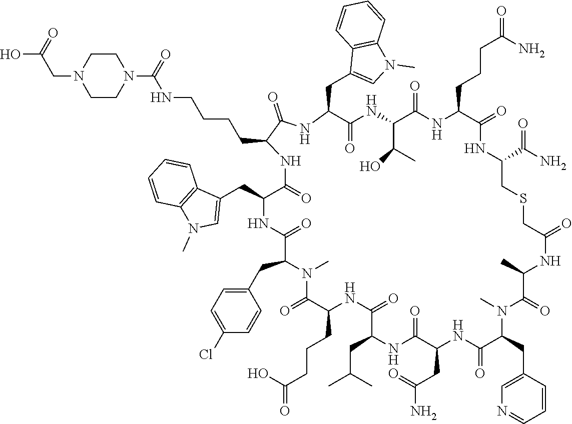 | HPLC-MS (Method A-B): 4.22 min ESI-MS m/z: 952.0 [M + 2H]2+ |
| PDC_EphA2- 00018528 (SEQ ID NO: 24) | 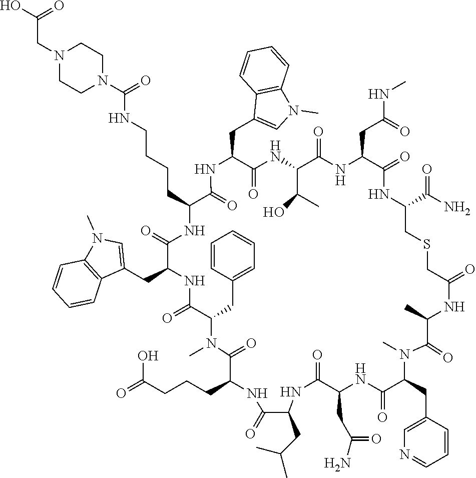 | HPLC-MS (Method A-B): 3.98 min ESI-MS m/z: 927.8 [M + 2H]2+ |
| PDC_EphA2- 00018529 (SEQ ID NO: 25) | 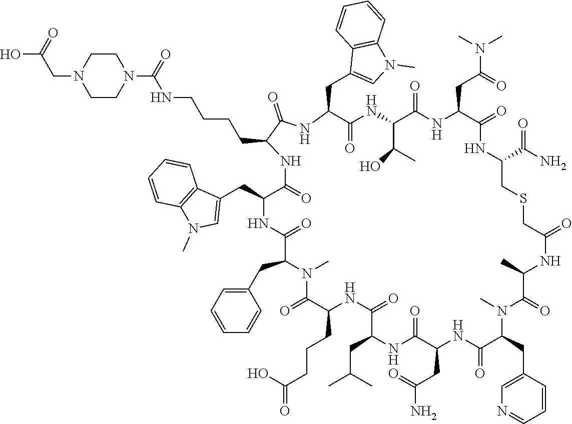 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CC(=O)N(C)C)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](Cc2ccccc2)N(C)C(=O)[C@H](CCCC(=O)O)NC1=O] | HPLC-MS (Method A-B): 4.13 min ESI-MS m/z: 934.4 [M + 2H]2+ |
| PDC_EphA2- 00018530 (SEQ ID NO: 26) | 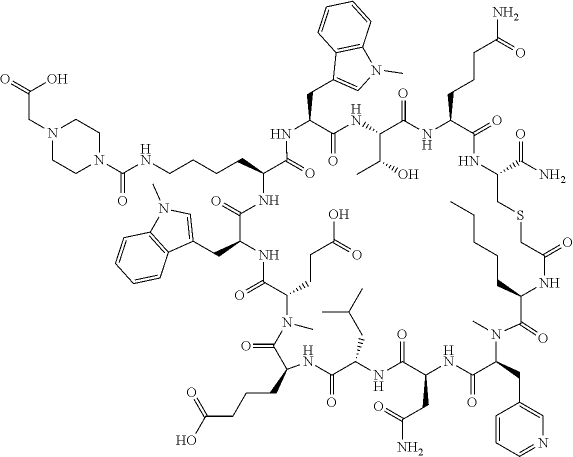 [SMILES: CCCCC[C@H]1NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(=O)O)NC(=O)[C@H](CC(C)C)NC(=O)[C@H](CC(N)=O)NC(=O)[C@H](Cc2cccnc2)N(C)C1=O] | HPLC-MS (Method A-B): 3.65 min ESI-MS m/z: 952.9 [M + 2H]2+ |
| PDC_EphA2- 00018531 (SEQ ID NO: 27) | 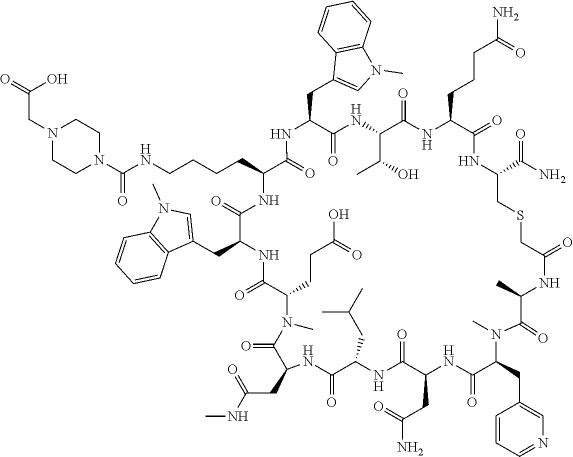 [SMILES: CNC(=O)C[C@@H]1NC(=O)[C@H](CC(C)C)NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C1=O] | HPLC-MS (Method A-B): 3.06 min ESI-MS m/z: 917.8 [M + 2H]2+ |
| PDC_EphA2- 00018532 (SEQ ID NO: 28) | 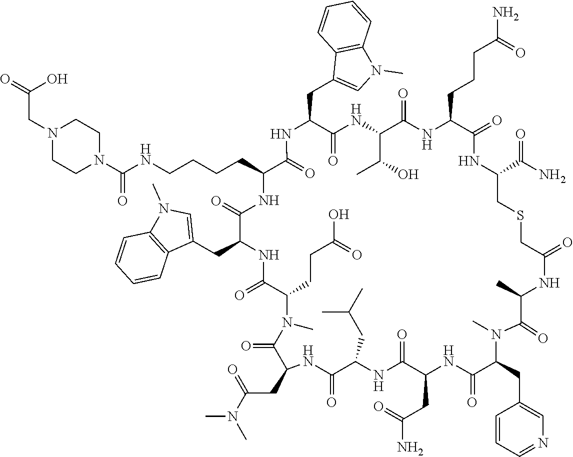 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CC(=O)N(C)C)NC1=O] | HPLC-MS (Method A-B): 3.30 min ESI-MS m/z: 924.9 [M + 2H]2+ |
| PDC_EphA2- 00018533 (SEQ ID NO: 29) | 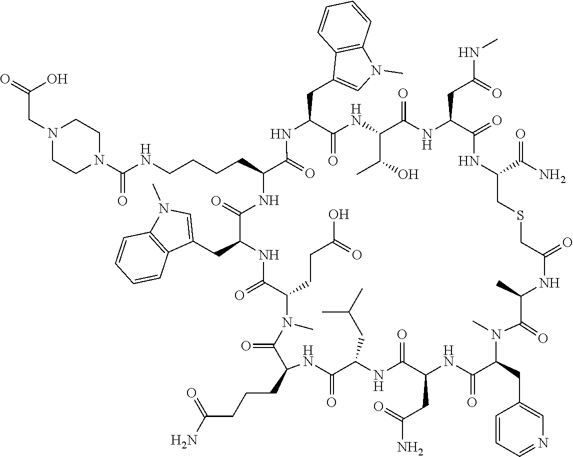 [SMILES: CNC(=O)C[C@@H]1NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(N)=O)NC(=O)[C@H](CC(C)C)NC(=O)[C@H](CC(N)=O)NC(=O)[C@H](Cc2cccnc2)N(C)C(=O)[C@@H](C)NC(=O)CSC[C@@H](C(N)=O)NC1=O] | HPLC-MS (Method A-B): 3.09 min ESI-MS m/z: 917.8 [M + 2H]2+ |
| PDC_EphA2- 00018534 (SEQ ID NO: 30) | 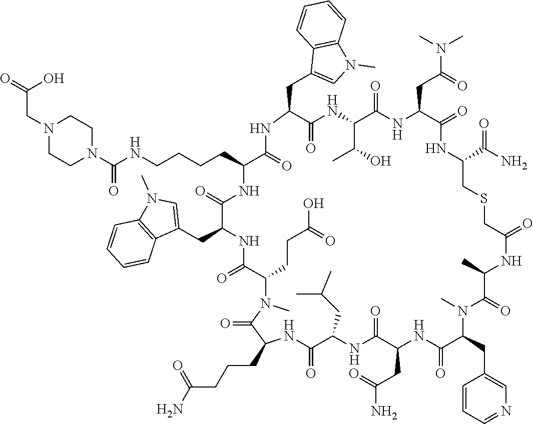 | HPLC-MS (Method A-B): 3.10 min ESI-MS m/z: 925.3 [M + 2H]2+ |
| PDC_EphA2- 00018535 (SEQ ID NO: 31) | 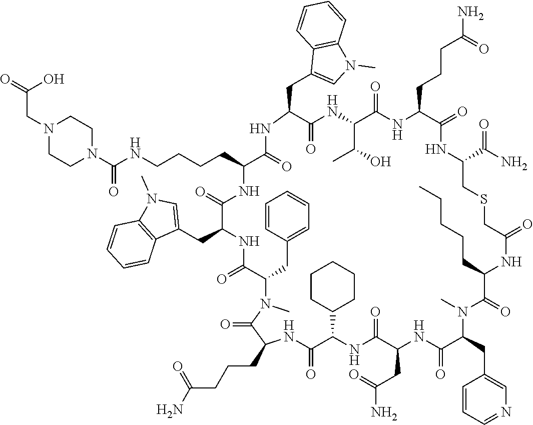 | HPLC-MS (Method A-B): 4.51 min ESI-MS m/z: 975.3 [M + 2H]2+ |
| PDC_EphA2- 00018536 (SEQ ID NO: 32) | 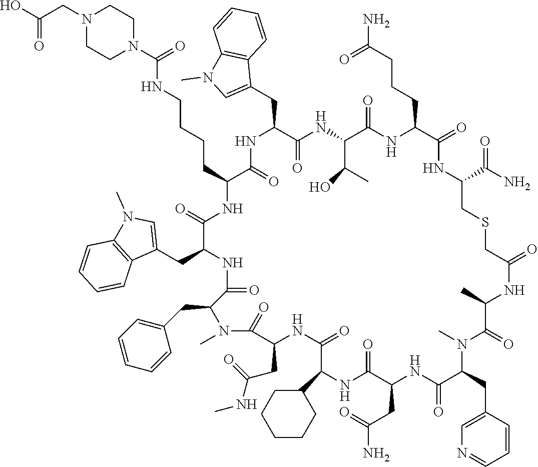 | HPLC-MS (Method A-B): 4.14 min ESI-MS m/z: 940.2 [M + 2H]2+ |
| PDC_EphA2- 00018537 (SEQ ID NO: 33) | 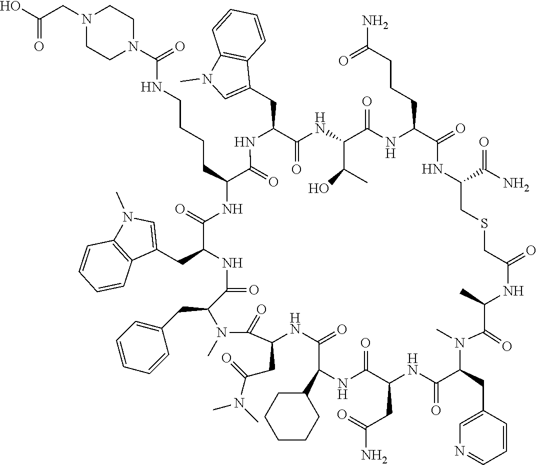 | HPLC-MS (Method A-B): 4.36 min ESI-MS m/z: 947.3 [M + 2H]2+ |
| PDC_EphA2- 00018538 (SEQ ID NO: 34) | 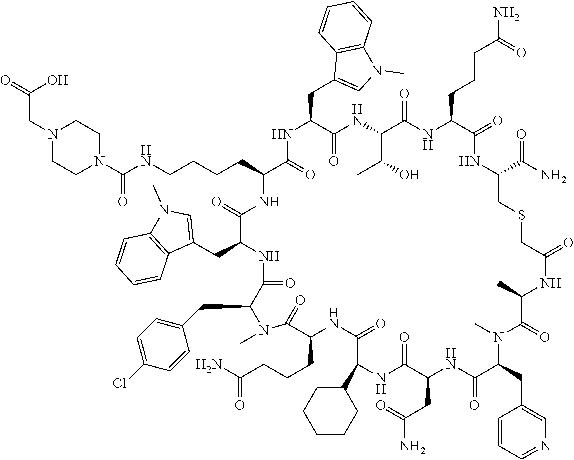 | HPLC-MS (Method A-B): 4.25 min ESI-MS m/z: 964.5 [M + 2H]2+ |
| PDC_EphA2- 00018539 (SEQ ID NO: 35) | 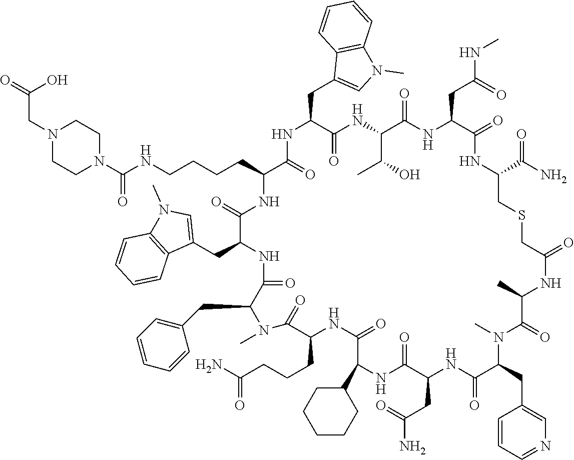 | HPLC-MS (Method A-B): 4.04 min ESI-MS m/z: 940.2 [M + 2H]2+ |
| PDC_EphA2- 00018540 (SEQ ID NO: 36) | 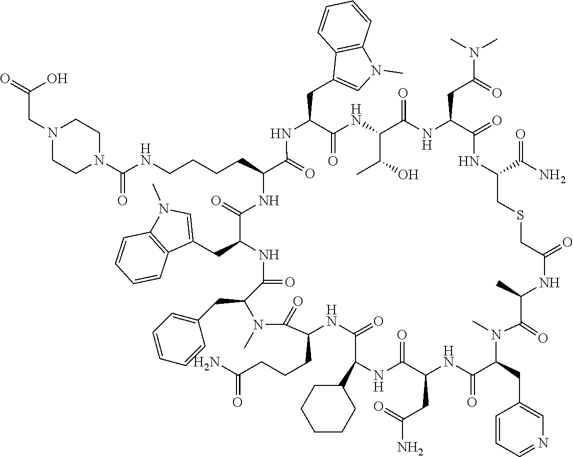 | HPLC-MS (Method A-B): 4.14 min ESI-MS m/z: 947.3 [M + 2H]2+ |
| PDC_EphA2- 00019421 (SEQ ID NO: 37) | 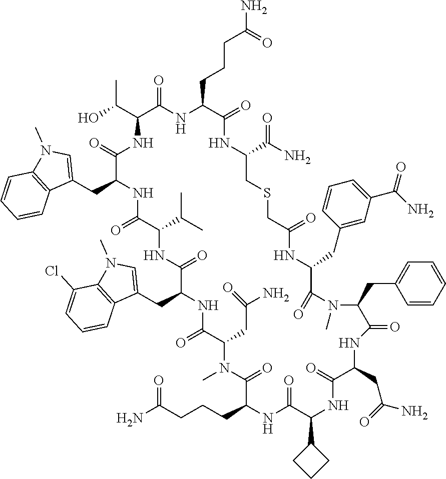 [SMILES: CC(C)[C@@H]1NC(=O)[C@H](Cc2cn(C)c3c(Cl)cccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-B): 5.91 min ESI-MS m/z: 893.0 [M + 2H]2+ |
| PDC_EphA2- 00019422 (SEQ ID NO: 38) | 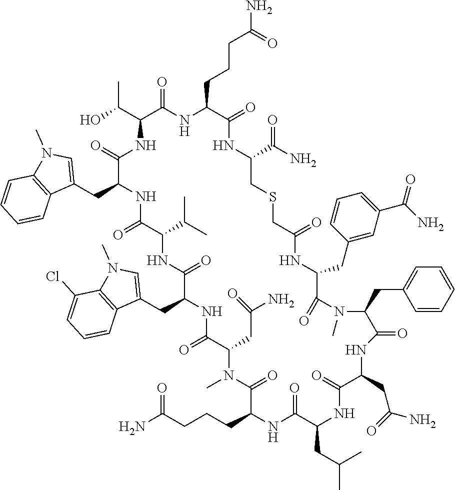 [SMILES: CC(C)C[C@@H]1NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](C(C)C)NC(=O)[C@H](Cc2cn(C)c3c(Cl)cccc23)NC(=O)[C@H](CC(N)=O)N(C)C(=O)[C@H](CCCC(N)=O)NC1=O] | HPLC-MS (Method A-B): 6.00 min ESI-MS m/z: 894.1 [M + 2H]2+ |
| PDC_EphA2- 00019423 (SEQ ID NO: 39) | 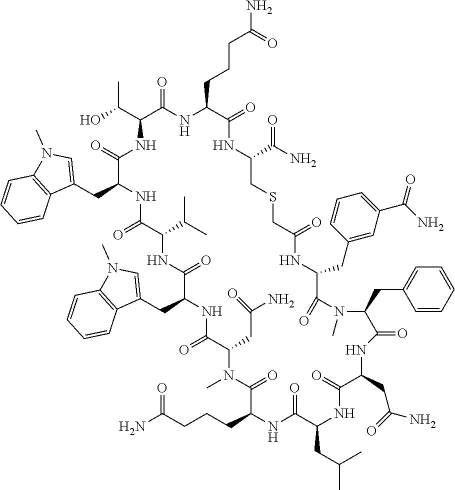 [SMILES: CC(C)C[C@@H]1NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](C(C)C)NC(=O)[C@H](Cc2cn(C)c3ccccc23)NC(=O)[C@H](CC(N)=O)N(C)C(=O)[C@H](CCCC(N)=O)NC1=O] | HPLC-MS (Method A-B): 5.46 min ESI-MS m/z: 876.9 [M + 2H]2+ |
| PDC_EphA2- 00019424 (SEQ ID NO: 40) | 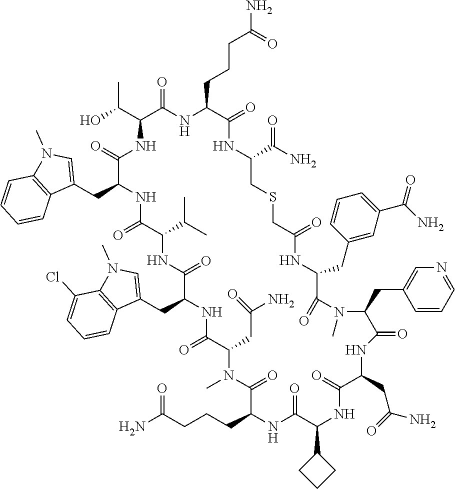 | HPLC-MS (Method A-B): 3.93 min ESI-MS m/z: 893.6 [M + 2H]2+ |
| PDC_EphA2- 00019425 (SEQ ID NO: 41) | 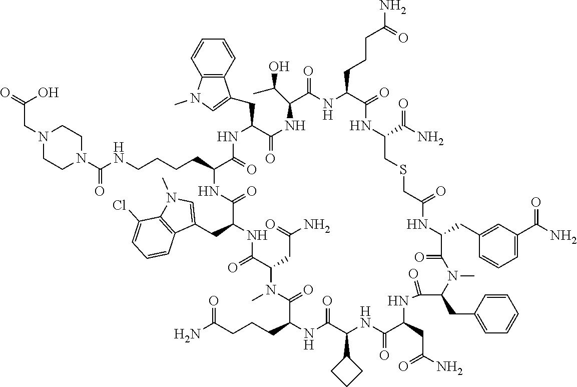 | HPLC-MS (Method A-B): 4.96 min ESI-MS m/z: 992.8 [M + 2H]2+ |
| PDC_EphA2- 00019426 (SEQ ID NO: 42) | 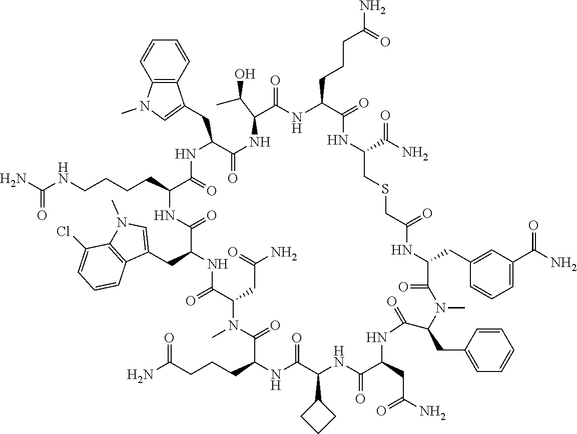 [SMILES: C[C@@H](O)[C@@H]1NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCC(N)=O)NC1=O] | HPLC-MS (Method A-B): 5.52 min ESI-MS m/z: 929.1 [M + 2H]2+ |
| PDC_EphA2- 00019427 (SEQ ID NO: 43) | 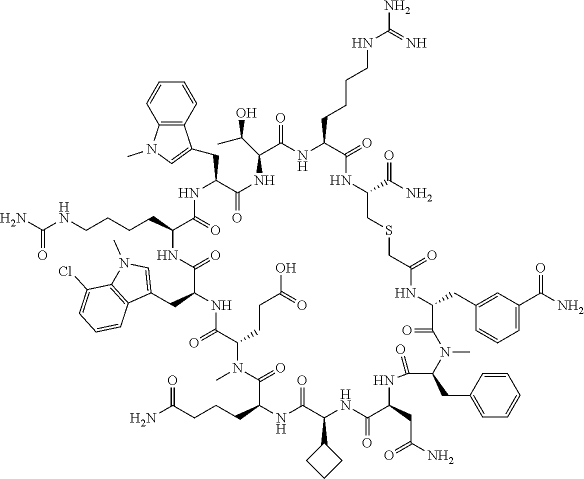 [SMILES: C[C@@H](O)[C@@H]1NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@H](CCC(=O)O)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCCNC(=N)N)NC1=O] | HPLC-MS (Method A-B): 4.89 min ESI-MS m/z: 950.6 [M + 2H]2+ |
| PDC_EphA2- 00019428 (SEQ ID NO: 44) | 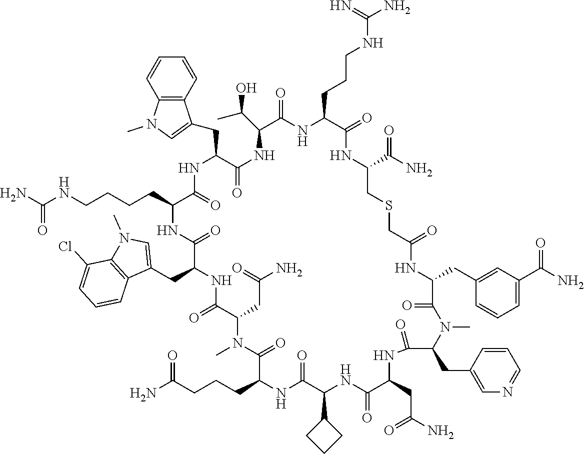 [SMILES: C[C@@H](O)[C@@H]1NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCNC(=N)N)NC1=O] | HPLC-MS (Method A-B): 3.17 min ESI-MS m/z: 936.6 [M + 2H]2+ |
| PDC_EphA2- 00019429 (SEQ ID NO: 45) | 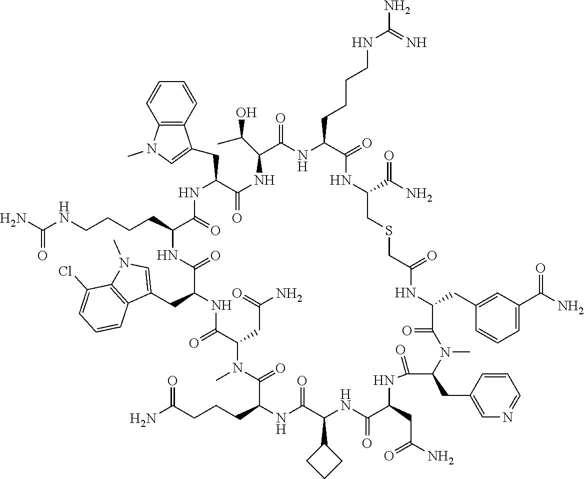 | HPLC-MS (Method A-B): 3.18 min ESI-MS m/z: 943.7 [M + 2H]2+ |
| PDC_EphA2- 00019430 (SEQ ID NO: 46) | 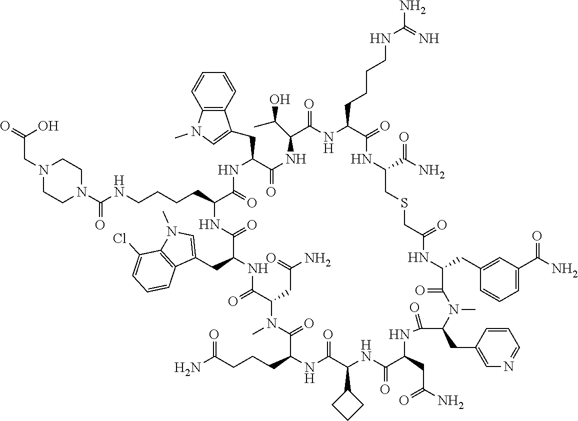 | HPLC-MS (Method A-B): 2.83 min ESI-MS m/z: 1007.2 [M + 2H]2+ |
| PDC_EphA2- 00019431 (SEQ ID NO: 47) | 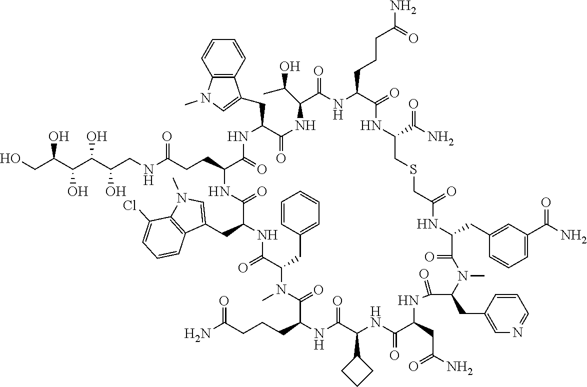 [SMILES: C[C@@H](O)[C@@H]1NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3c(Cl)cccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCC(N)=O)NC1=O] | HPLC-MS (Method A-B): 6.04 min ESI-MS m/z: 1006.1 [M + 2H]2+ |
| PDC_EphA2- 00019432 (SEQ ID NO: 48) | 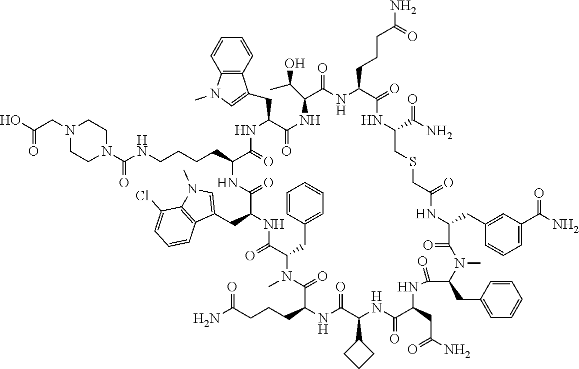 [SMILES: C[C@@H](O)[C@@H]1NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](Cc2cccc(C(N)=O)c2)NC(=O)CSC[C@@H](C(N)=O)NC(=O)[C@H](CCCC(N)=O)NC1=O] | HPLC-MS (Method A-B): 5.74 min ESI-MS m/z: 1009.1 [M + 2H]2+ |
| PDC_EphA2- 00019433 (SEQ ID NO: 49) | 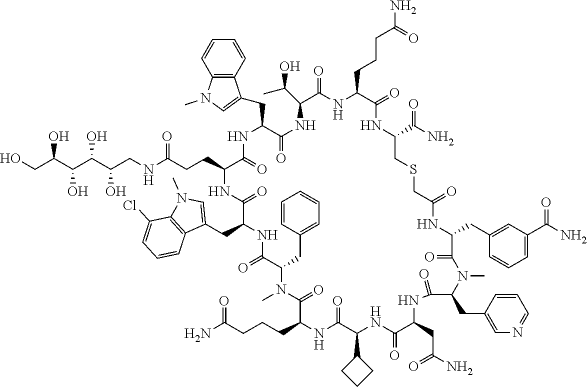 [SMILES: C[C@@H](O)[C@@H]1NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3c(Cl)cccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCC(N)=O)NC1=O] | HPLC-MS (Method A-B): 4.17 min ESI-MS m/z: 1006.6 [M + 2H]2+ |
| PDC_EphA2- 00019434 (SEQ ID NO: 50) | 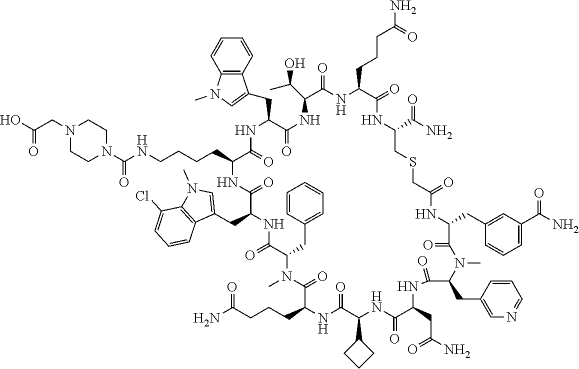 [SMILES: C[C@@H](O)[C@@H]1NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@H](Cc2ccccc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCC(N)=O)NC1=O] | HPLC-MS (Method A-B): 4.10 min ESI-MS m/z: 1009.6 [M + 2H]2+ |
| PDC_EphA2- 00019435 (SEQ ID NO: 51) | 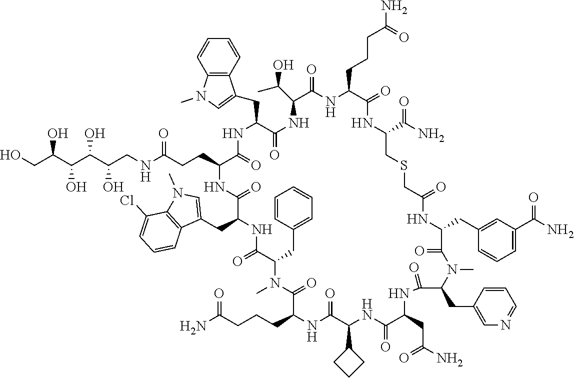 | HPLC-MS (Method A-B): 4.76 min ESI-MS m/z: 1006.7 [M + 2H]2+ |
| PDC_EphA2- 00019436 (SEQ ID NO: 52) | 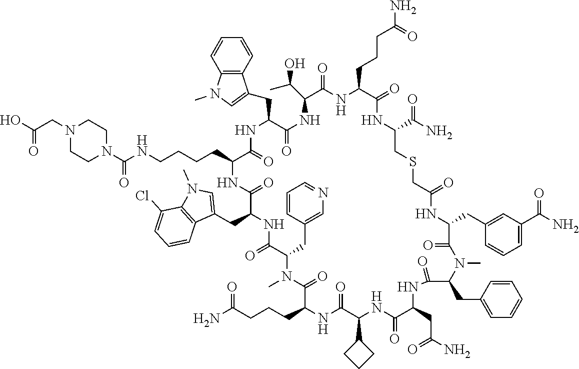 | HPLC-MS (Method A-B): 4.38 min ESI-MS m/z: 1010.0 [M + 2H]2+ |
| PDC_EphA2- 00019437 (SEQ ID NO: 53) | 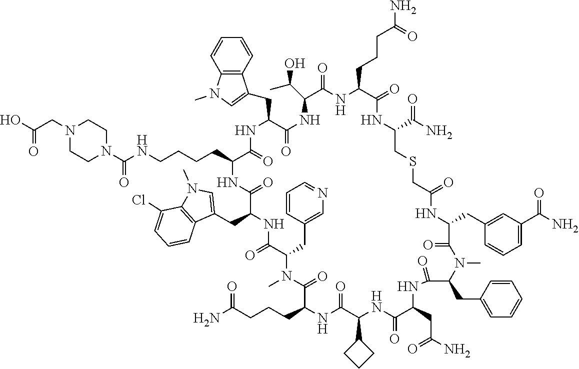 | HPLC-MS (Method A-B): 2.96 min ESI-MS m/z: 1007.1 [M + 2H]2+ |
| PDC_EphA2- 00019438 (SEQ ID NO: 54) | 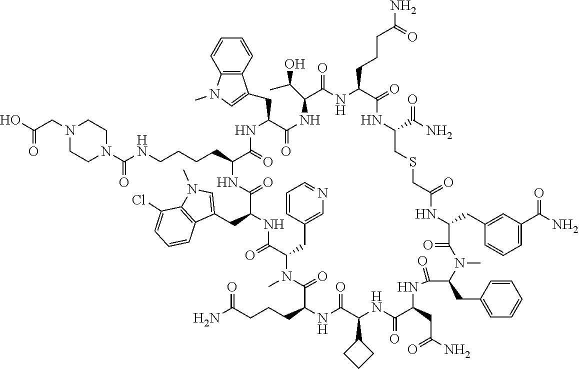 | HPLC-MS (Method A-B): 2.91 min ESI-MS m/z: 1010.1 [M + 2H]2+ |
| PDC_EphA2- 00019439 (SEQ ID NO: 55) | 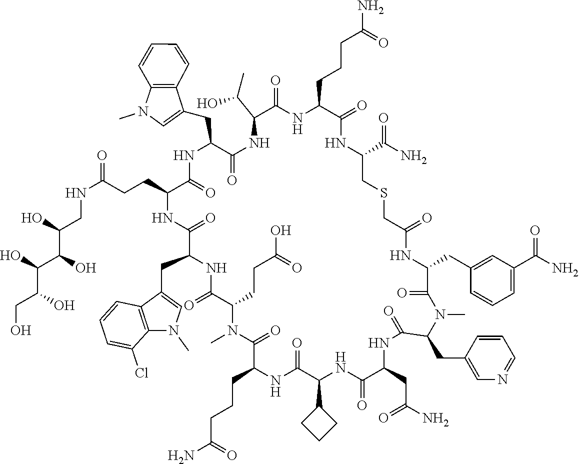 | HPLC-MS (Method A-B): 3.30 min ESI-MS m/z: 998.0 [M + 2H]2+ |
| PDC_EphA2- 00019440 (SEQ ID NO: 56) | 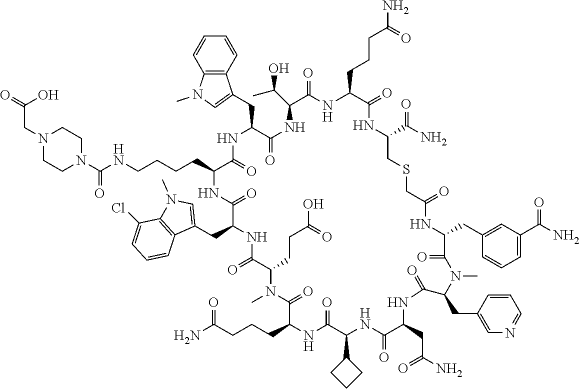 | HPLC-MS (Method A-B): 3.26 min ESI-MS m/z: 1001.0 [M + 2H]2+ |
| PDC_EphA2- 00019441 (SEQ ID NO: 57) | 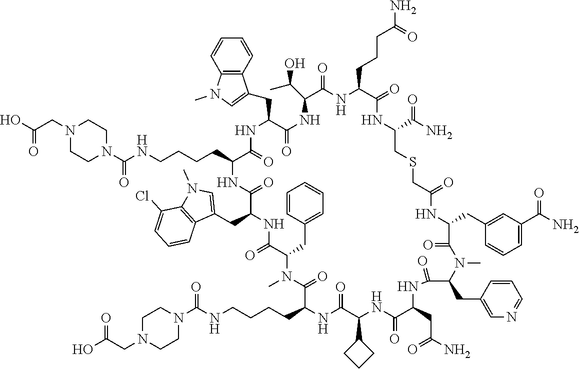 | HPLC-MS (Method A-B): 3.70 min ESI-MS m/z: 1001.0 [M + 3H]3+ |
| PDC_EphA2- 00019442 (SEQ ID NO: 58) | 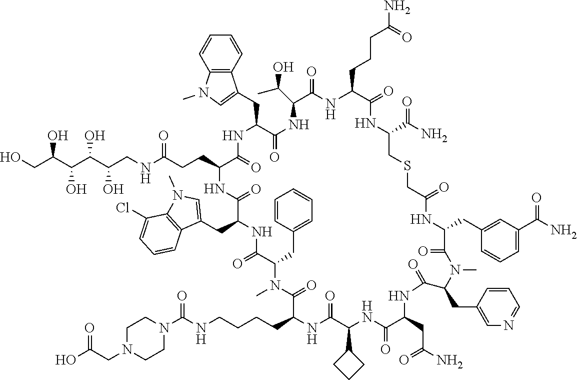 | HPLC-MS (Method A-B): 3.86 min ESI-MS m/z: 1084.7 [M + 2H]2+ |
| PDC_EphA2- 00026424 (SEQ ID NO: 59) | 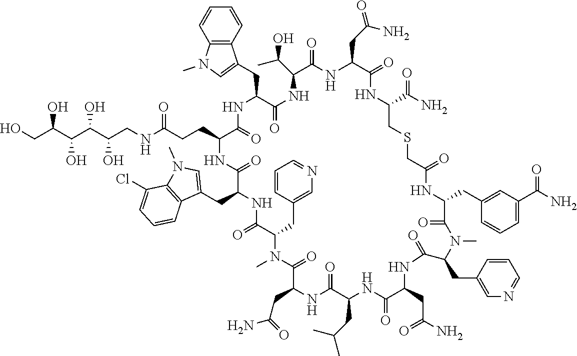 | HPLC-MS (Method A-B): 2.86 min ESI-MS m/z: 980.4 [M + 2H]2+ |
| PDC_EphA2- 00026425 (SEQ ID NO: 60) | 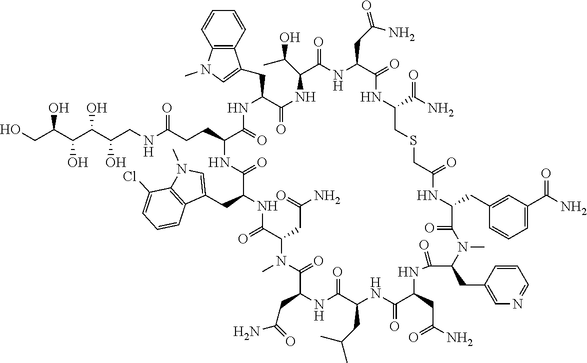 | HPLC-MS (Method A-B): 3.25 min ESI-MS m/z: 963.4 [M + 2H]2+ |
| PDC_EphA2- 00026426 (SEQ ID NO: 61) | 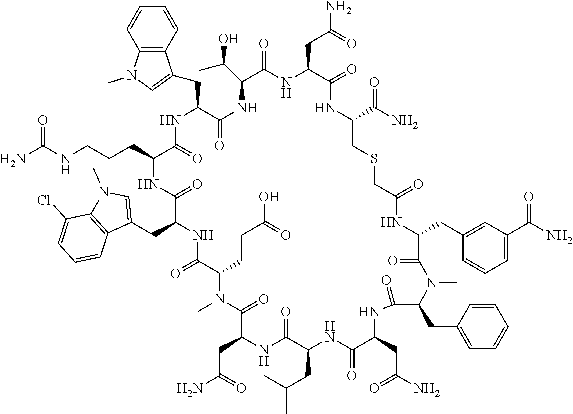 | HPLC-MS (Method A-B): 3.47 min ESI-MS m/z: 896.0 [M + 2H]2+ |
| PDC_EphA2- 00010011-C002 (SEQ ID NO: 201) | 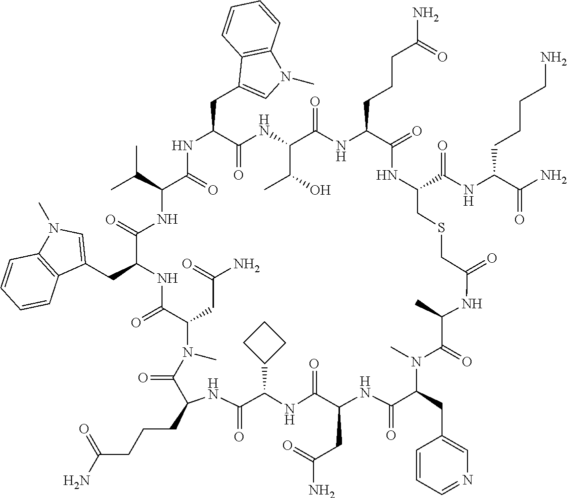 [SMILES: CC(C)[C@@H]1NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](C)NC(=O)CSC[C@@H](C(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-A): 5.50 min ESI-MS m/z: 880.9 [M + 2H]2+ |
| PDC_EphA2- 00010012-C002 (SEQ ID NO: 202) | 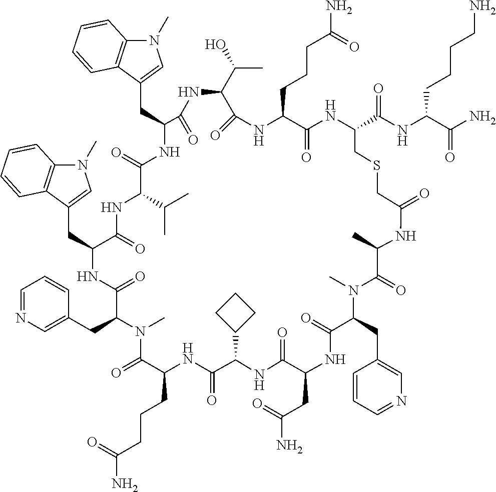 [SMILES: CC(C)[C@@H]1NC(=O)[C@H](Cc2cn(C)c3ccccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](C)NC(=O)CSC[C@@H](C(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-A): 5.24 min ESI-MS m/z: 898.0 [M + 2H]2+ |
| PDC_EphA2- 00013693-C002 (SEQ ID NO: 203) | 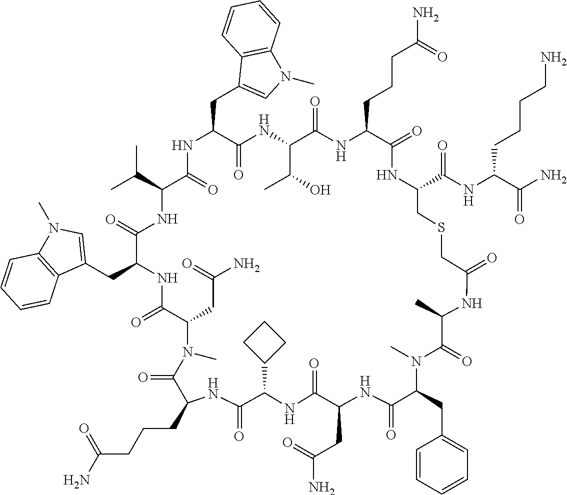 [SMILES: CC(C)[C@@H]1NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](C)NC(=O)CSC[C@@H](C(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-B): 4.34 min ESI-MS m/z: 880.3 [M + 2H]2+ |
| PDC_EphA2- 00022593-C002 (SEQ ID NO: 205) | 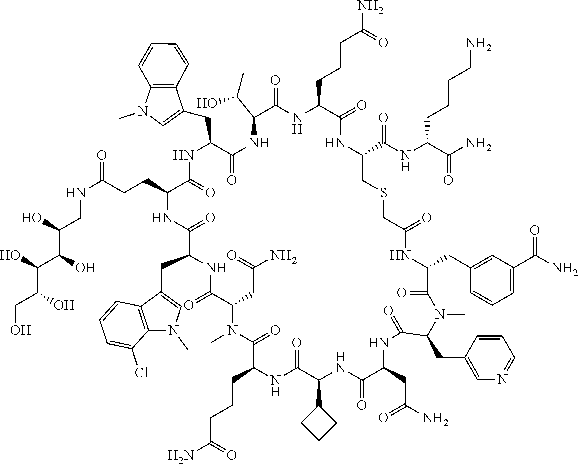 | HPLC-MS (Method A-A): 5.34 min ESI-MS m/z: 703.5 [M + 3H]3+ |
| PDC_EphA2- 00022594-C002 (SEQ ID NO: 206) | 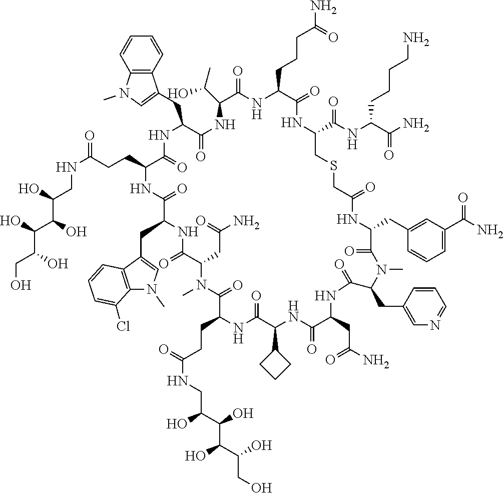 | HPLC-MS (Method A-A): 5.19 min ESI-MS m/z: 753.6 [M + 3H]3+ |
| PDC_EphA2- 00022595-C002 (SEQ ID NO: 207) | 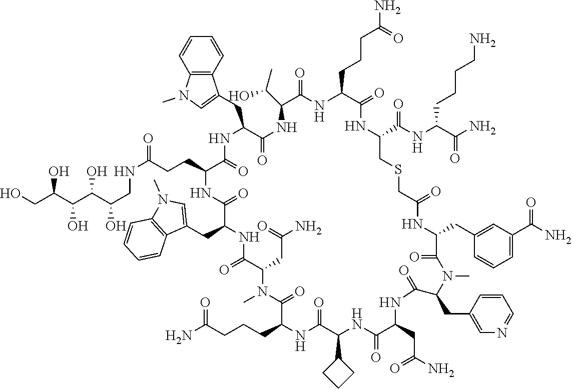 | HPLC-MS (Method A-A): 4.84 min ESI-MS m/z: 1037.2 [M + 2H]2+ |
| PDC_EphA2- 00022612-C002 (SEQ ID NO: 209) | 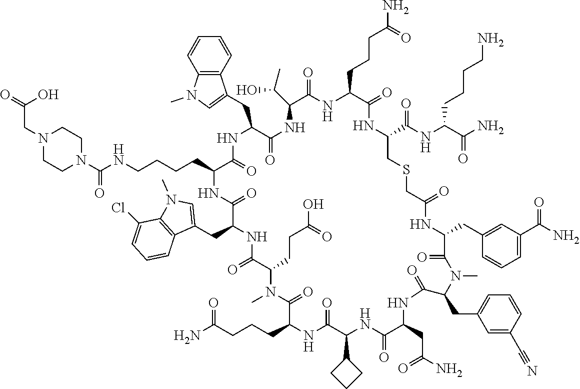 | HPLC-MS (Method A-B): 4.14 min ESI-MS m/z: 718.6 [M + 3H]3+ |
| PDC_EphA2- 00022613-C002 (SEQ ID NO: 210) | 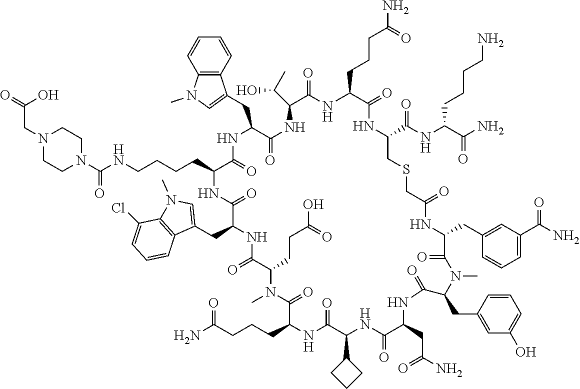 | HPLC-MS (Method A-B): 3.90 min ESI-MS m/z: 1072.2 [M + 2H]2+ |
| PDC_EphA2- 00022614-C002 (SEQ ID NO: 211) | 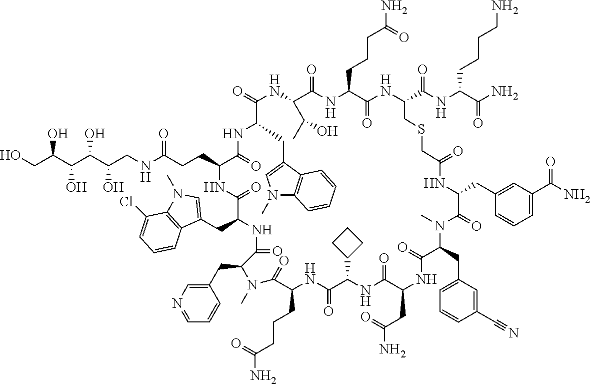 | HPLC-MS (Method A-B): 3.82 min ESI-MS m/z: 722.9 [M + 3H]3+ |
| PDC_EphA2- 00022615-C002 (SEQ ID NO: 212) | 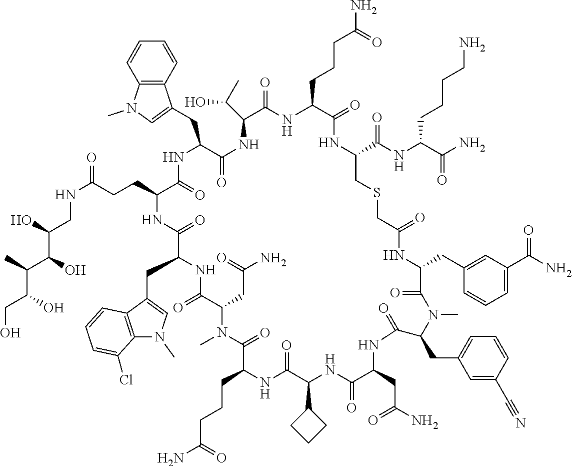 | HPLC-MS (Method A-B): 4.44 min ESI-MS m/z: 1066.2 [M + 2H]2+ |
| PDC_EphA2- 00013703-C002 (SEQ ID NO: 213) | 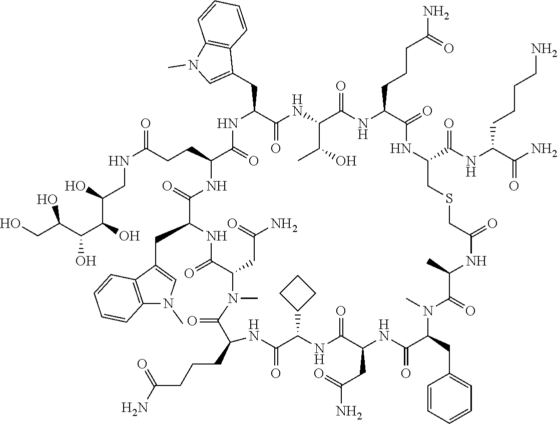 [SMILES: C[C@H]1NC(=O)CSC[C@@H](C(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C1=O] | HPLC-MS (Method A-B): 3.88 min ESI-MS m/z: 976.9 [M + 2H]2+ |
| PDC_EphA2- 00013704-C002 (SEQ ID NO: 214) | 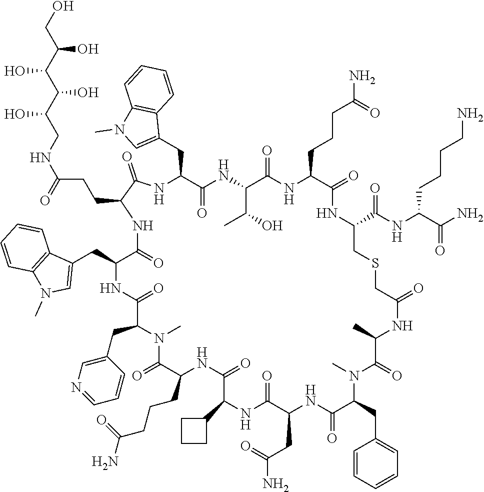 [SMILES: C[C@H]1NC(=O)CSC[C@@H](C(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3ccccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2ccccc2)N(C)C1=O] | HPLC-MS (Method A-B): 3.39 min ESI-MS m/z: 993.9 [M + 2H]2+ |
| PDC_EphA2- 00013705-C002 (SEQ ID NO: 215) | 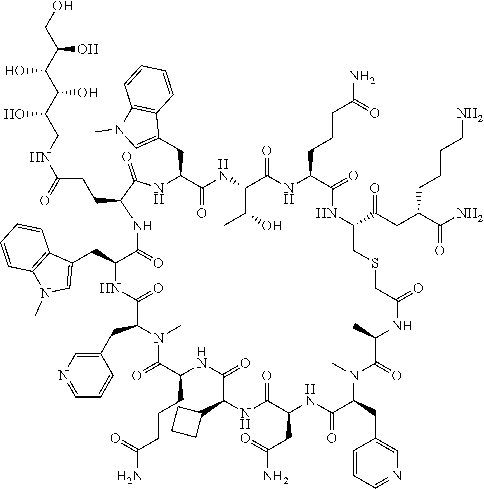 | HPLC-MS (Method A-A): 4.45 min ESI-MS m/z: 994.8 [M + 2H]2+ |
| PDC_EphA2- 00026424-C002 (SEQ ID NO: 216) | 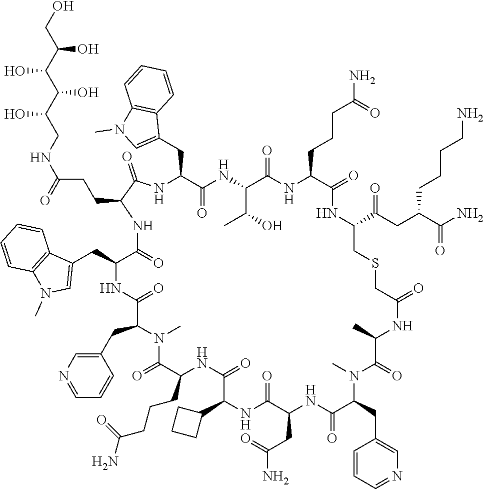 | HPLC-MS (Method A-A): 5.14 min ESI-MS m/z: 696.8 [M + 3H]3+ |
| PDC_EphA2- 00026425-C002 (SEQ ID NO: 217) | 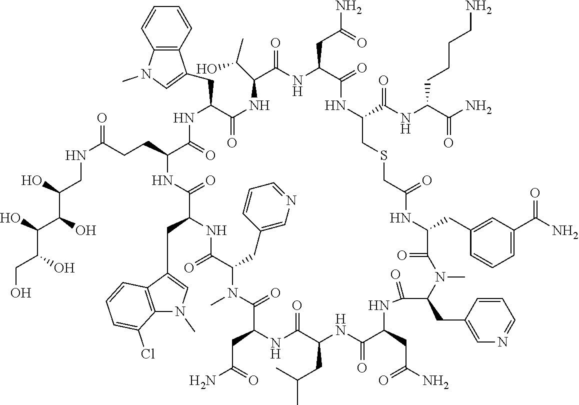 | HPLC-MS (Method A-B): 3.01 min ESI-MS m/z: 1027.2 [M + 2H]2+ |
| PDC_EphA2- 00026426-C002 (SEQ ID NO: 218) | 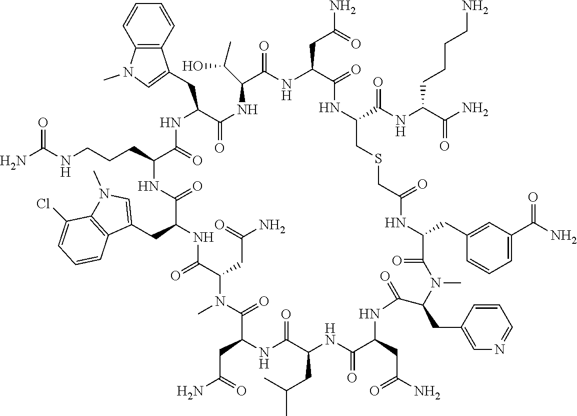 | HPLC-MS (Method A-B): 3.19 min ESI-MS m/z: 959.7 [M + 2H]2+ |
| PDC_EphA2- 00010011-C003 (SEQ ID NO: 180) | 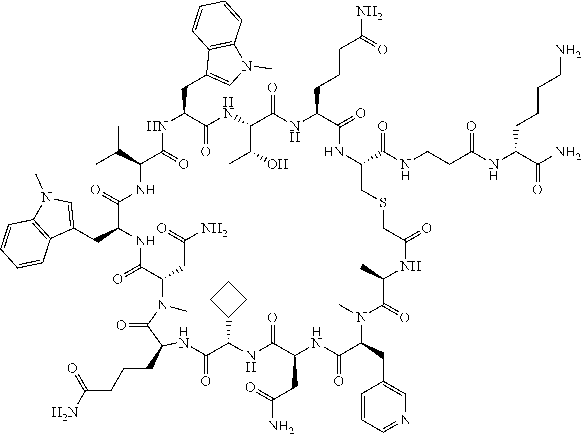 [SMILES: CC(C)[C@@H]1NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](C)NC(=O)CSC[C@@H](C(=O)NCCC(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-B): 3.10 min ESI-MS m/z: 916.5 [M + 2H]2+ |
| PDC_EphA2- 00010012-C003 (SEQ ID NO: 181) | 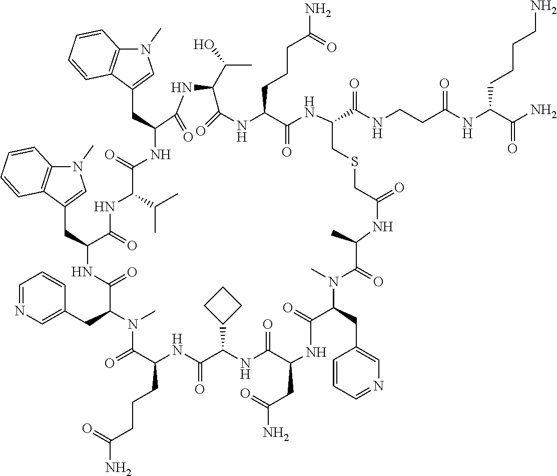 | HPLC-MS (Method A-A): 5.17 min ESI-MS m/z: 622.6 [M + 3H]3+ |
| PDC_EphA2- 00013693-C003 (SEQ ID NO: 182) | 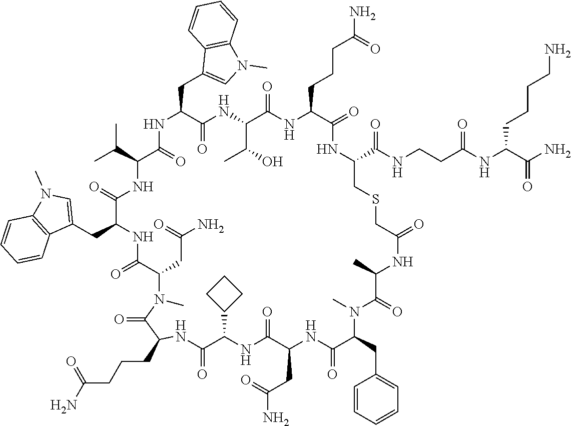 [SMILES: CC(C)[C@@H]1NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](C)NC(=O)CSC[C@@H](C(=O)NCCC(=O)N[C@H](CCCCN)C(N)=O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A-B): 4.27 min ESI-MS m/z: 915.9 [M + 2H]2+ |
| PDC_EphA2- 00026521 (SEQ ID NO: 62) | 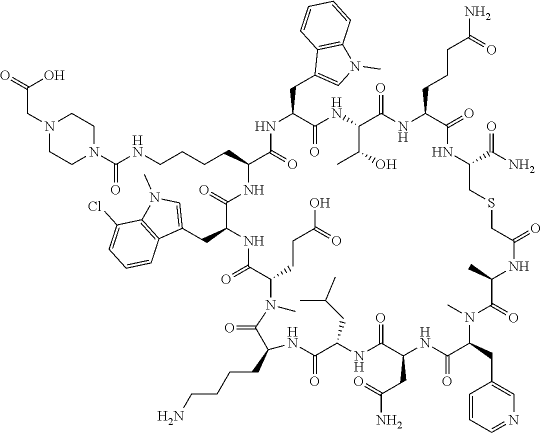 | HPLC-MS (Method A-A): 5.80 min ESI-MS m/z: 623.8 [M + 3H]3+ |
| PDC_EphA2- 00026522 (SEQ ID NO: 63) | 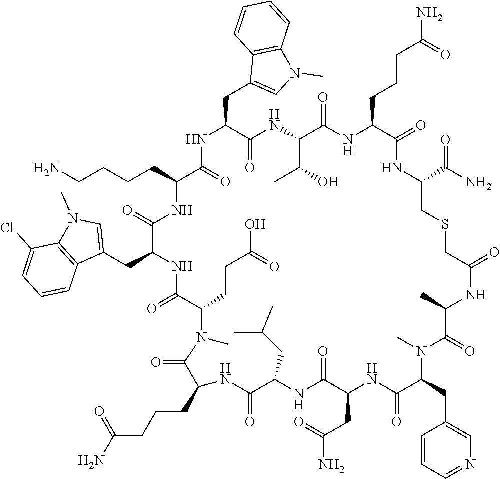 | HPLC-MS (Method A-A): 6.04 min ESI-MS m/z: 857.1 [M + 2H]2+ |
| PDC_EphA2- 00026523 (SEQ ID NO: 64) | 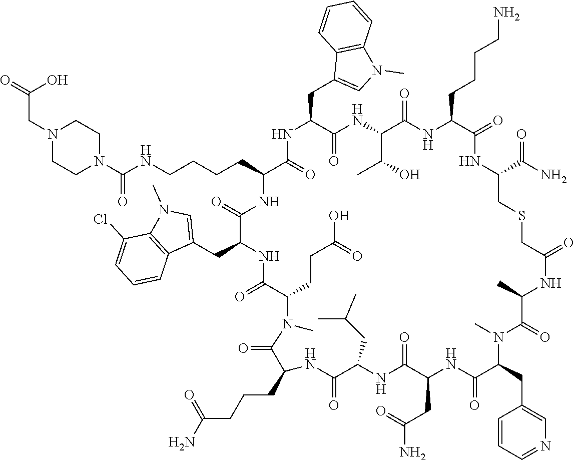 | HPLC-MS (Method A-A): 5.83 min ESI-MS m/z: 623.8 [M + 3H]3+ |
| PDC_EphA2- 00026524 (SEQ ID NO: 65) | 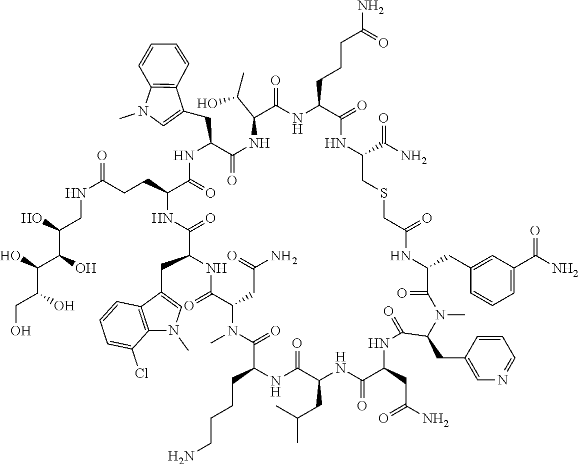 | HPLC-MS (Method A-A): 5.83 min ESI-MS m/z: 984.2 [M + 2H]2+ |
| PDC_EphA2- 00026525 (SEQ ID NO: 66) | 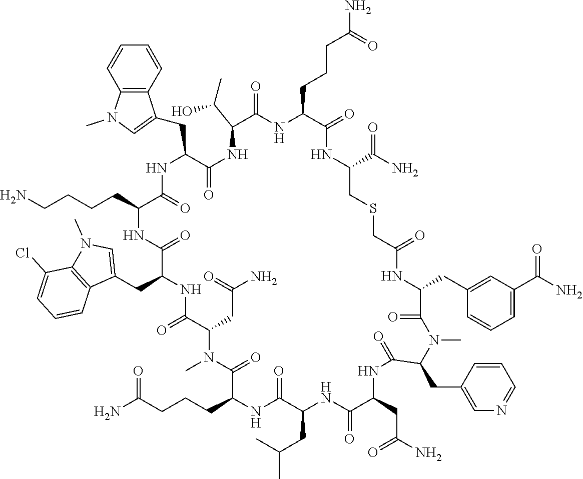 [SMILES: CC(C)C[C@@H]1NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCN)NC(=O)[C@H](Cc2cn(C)c3c(Cl)cccc23)NC(=O)[C@H](CC(N)=O)N(C)C(=O)[C@H](CCCC(N)=O)NC1=O] | HPLC-MS (Method A-B): 3.28 min ESI-MS m/z: 909.1 [M + 2H]2+ |
| PDC_EphA2- 00026526 (SEQ ID NO: 67) | 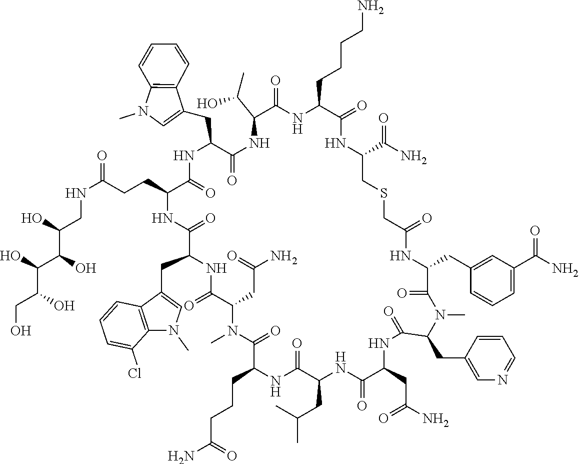 | HPLC-MS (Method A-A): 5.84 min ESI-MS m/z: 984.2 [M + 2H]2+ |
| PDC_EphA2- 00026527 (SEQ ID NO: 68) | 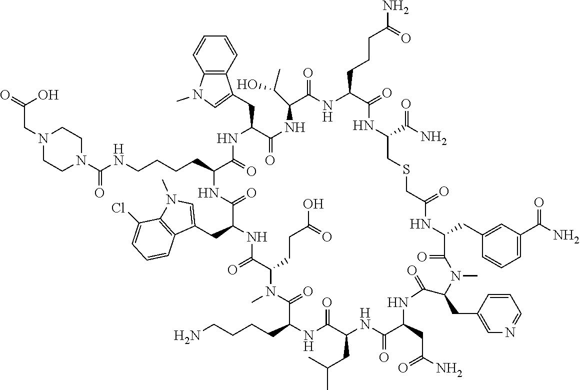 [SMILES: CC(C)C[C@@H]1NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@H](CCC(=O)O)N(C)C(=O)[C@H](CCCCN)NC1=O] | HPLC-MS (Method A-B): 3.13 min ESI-MS m/z: 994.6 [M + 2H]2+ |
| PDC_EphA2- 00026528 (SEQ ID NO: 69) | 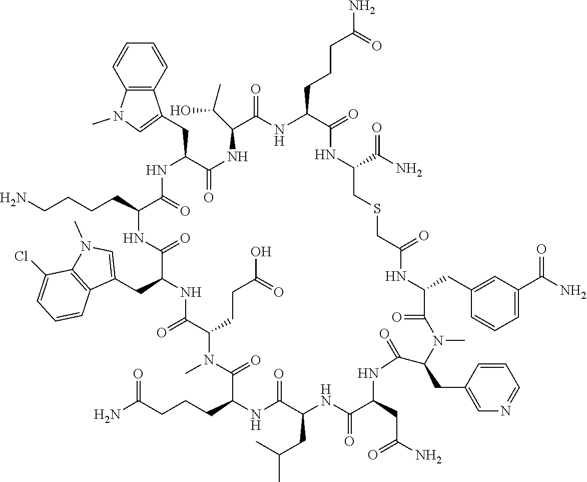 [SMILES: CC(C)C[C@@H]1NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCN)NC(=O)[C@H](Cc2cn(C)c3c(Cl)cccc23)NC(=O)[C@H](CCC(=O)O)N(C)C(=O)[C@H](CCCC(N)=O)NC1=O] | HPLC-MS (Method A-B): 3.40 min ESI-MS m/z: 916.5 [M + 2H]2+ |
| PDC_EphA2- 00026529 (SEQ ID NO: 70) | 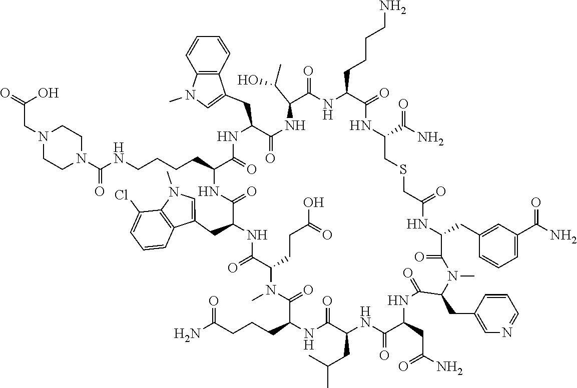 | HPLC-MS (Method A-B): 3.17 min ESI-MS m/z: 663.5 [M + 3H]3+ |
| PDC_EphA2- 00027094-C002 (SEQ ID NO: 252) | 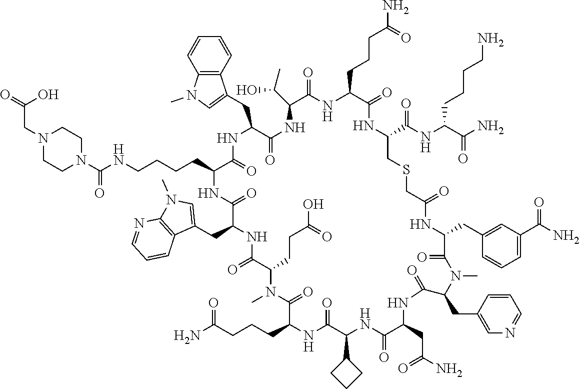 | HPLC-MS (Method A-A): 4.31 min ESI-MS m/z: 699.0 [M + 3H]3+ |
| PDC_EphA2- 00027095-C002 (SEQ ID NO: 253) | 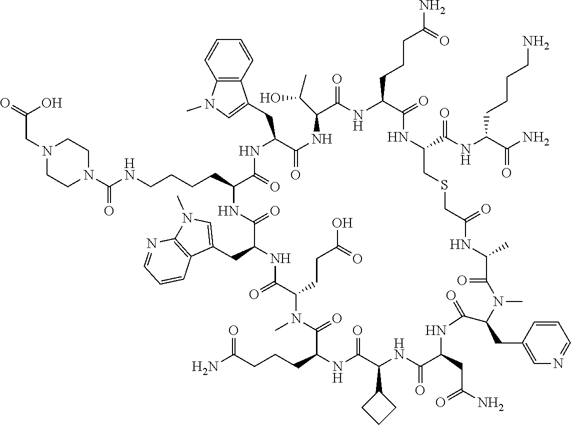 | HPLC-MS (Method A-A): 4.18 min ESI-MS m/z: 659.3 [M + 3H]3+ |
| PDC_EphA2- 00027092 (SEQ ID NO: 256) | 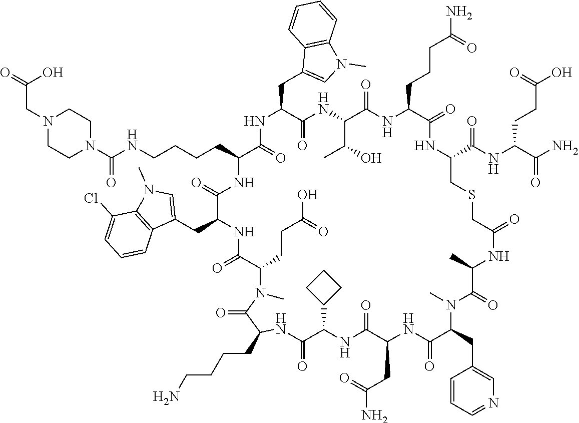 | HPLC-MS (Method A-A): 5.59 min ESI-MS m/z: 666.1 [M + 3H]3+ |

### Example B8

The following Examples were prepared by following similar methods described in the examples above. The amino acid sequence for each peptide was shown in Table B6.

TABLE B8

| Example No. | Structure | Analytics |
|---|---|---|
| PDC_EphA2- 00026624 (SEQ ID NO: 219) | 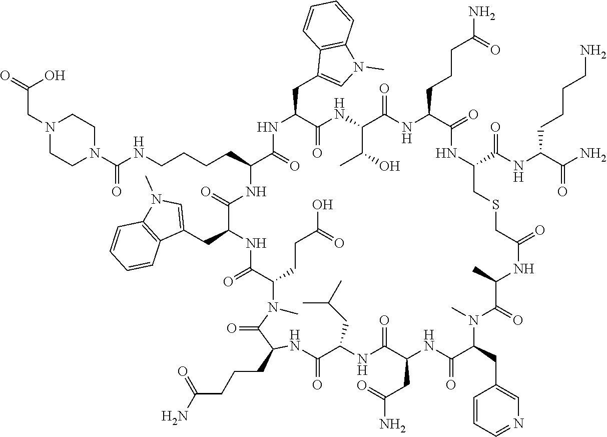 | HPLC-MS (Method A-A): 5.23 min ESI-MS m/z: 660.0 [M + 3H]3+ |
| PDC_EphA2- 00026625 (SEQ ID NO: 220) | 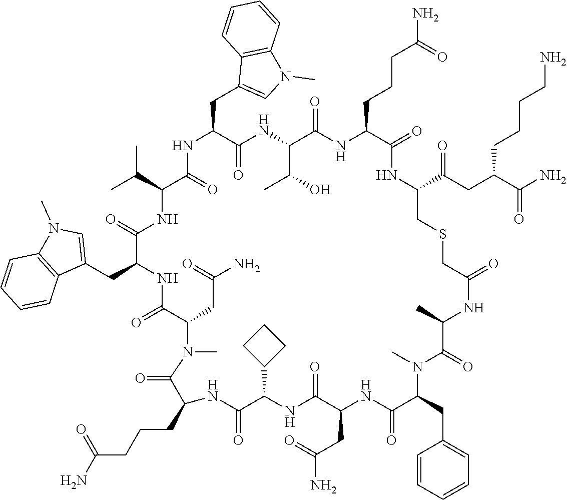 | HPLC-MS (Method A-B): 4.56 min ESI-MS m/z: 880.9 [M + 2H]2+ |
| PDC_EphA2- 00026627 (SEQ ID NO: 222) | 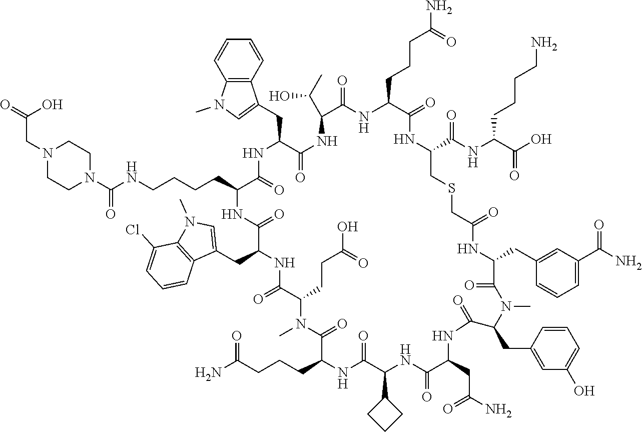 | HPLC-MS (Method A-B): 3.96 min ESI-MS m/z: 1072.8 [M + 2H]2+ |
| PDC_EphA2- 00027090 (SEQ ID NO: 254) | 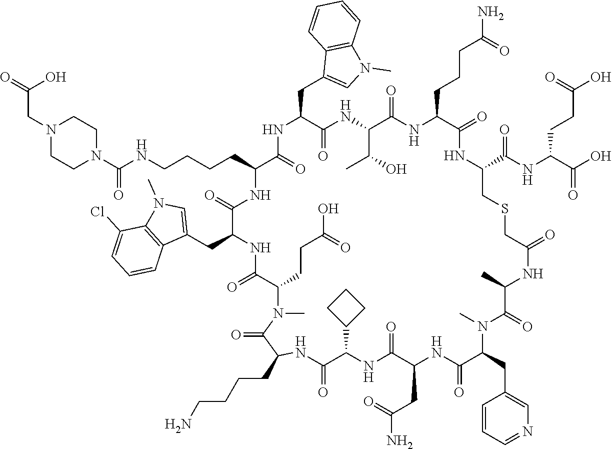 | HPLC-MS (Method A-A): 5.65 min ESI-MS m/z: 666.5 [M + 3H]3+ |
| PDC_EphA2- 00027091 (SEQ ID NO: 255) | 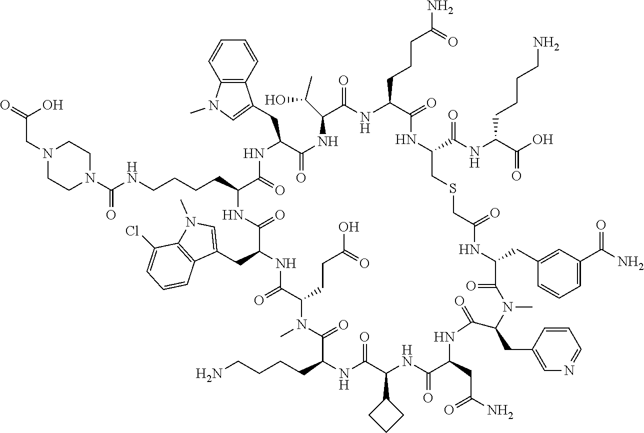 | HPLC-MS (Method A-A): 5.71 min ESI-MS m/z: 706.2 [M + 3H]3+ |

### Example B9: PDC_EphA2-00007196-C004 (SEQ ID No: 115 and 224)

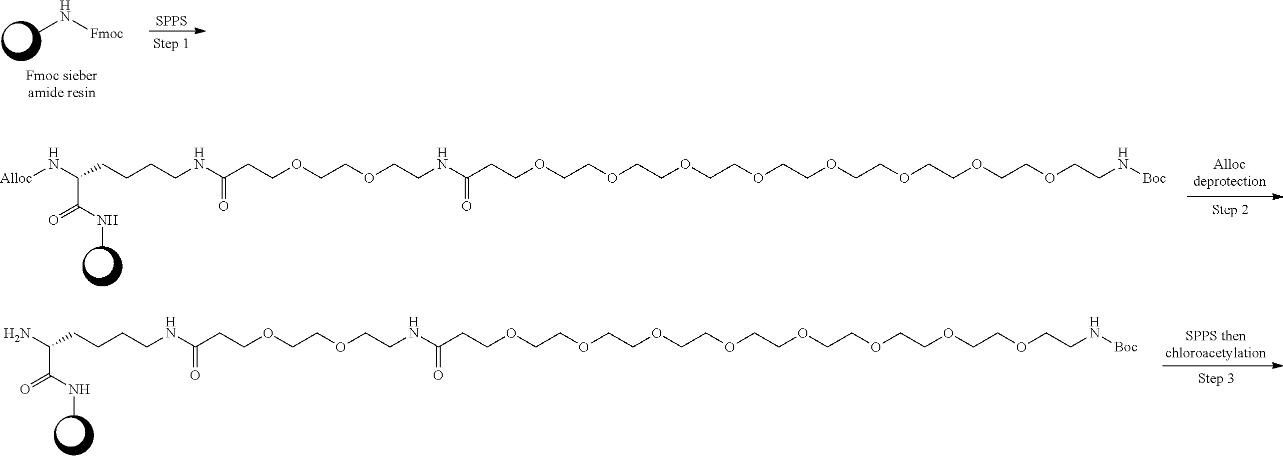

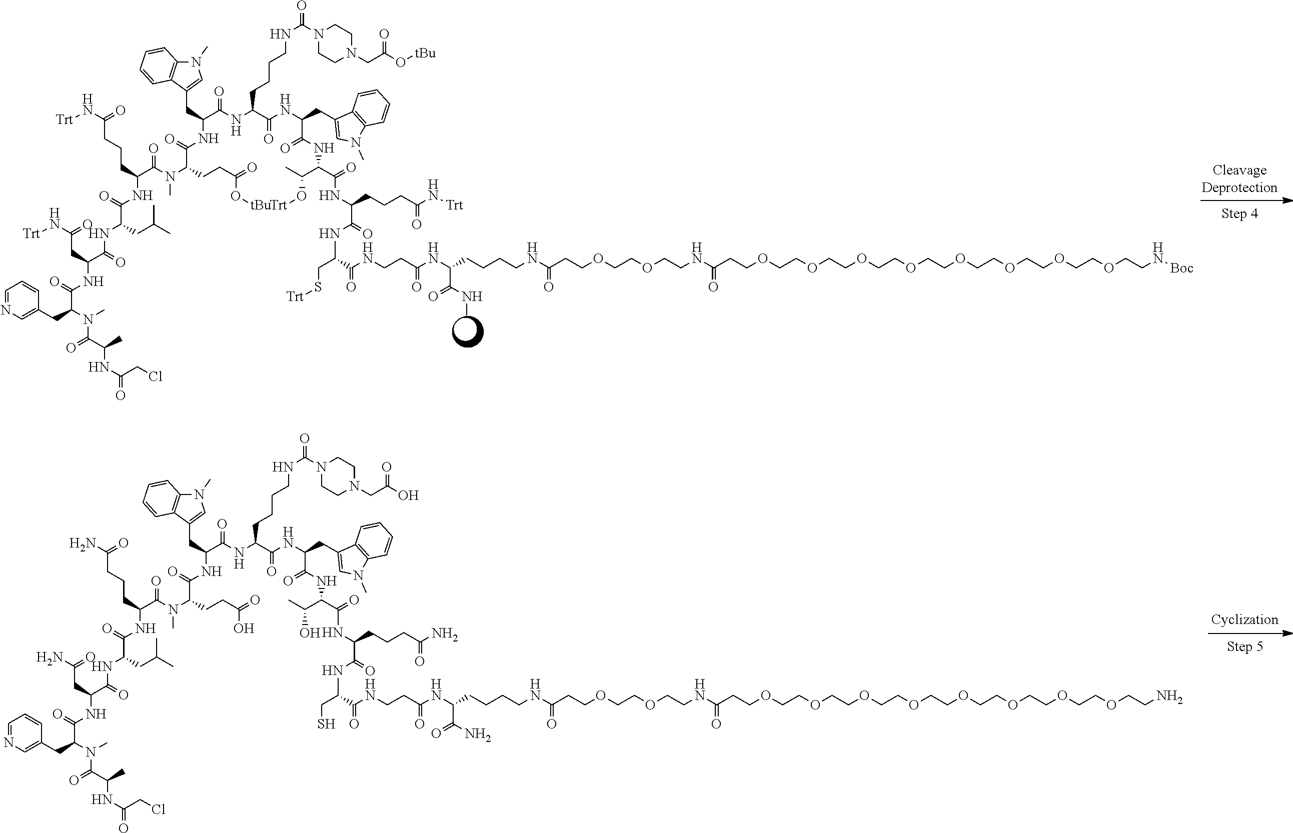

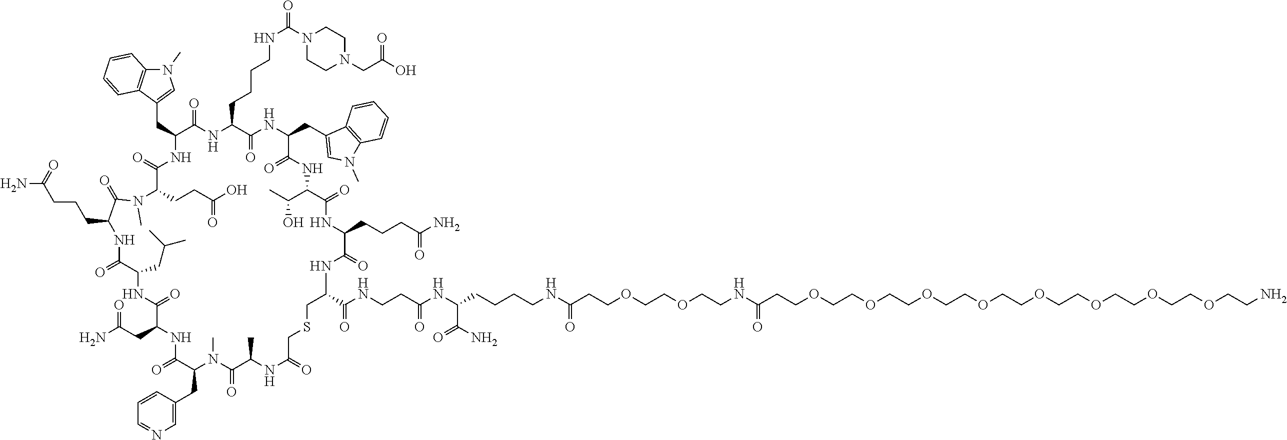 [SMILES: CC(C)C[C@@H]1NC(=O)[C@H](CC(N)=O)NC(=O)[C@H](Cc2cccnc2)N(C)C(=O)[C@@H](C)NC(=O)CSC[C@@H](C(=O)NCCC(=O)N[C@H](CCCCNC(=O)CCOCCOCCNC(=O)CCOCCOCCOCCOCCOCCOCCOCCOCCN)C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3ccccc23)NC(=O)[C@H](CCC(=O)O)N(C)C(=O)[C@H](CCCC(N)=O)NC1=O]

Intermediates on-resin were synthesized in the analogous manner to the step 1 of the scheme A. The intermediate peptide on-resin was synthesized on Fmoc-Sieber amide Resin (0.25 mmol) in the analogous manner to the step 1 of the scheme A. The resin was shaken with Pd(PPh3)4 (0.2 eq) and PhSiH3 (10 eq) at room temperature for 1 h. The peptide sequence was then synthesized on the intermediate following general peptide synthesis method A. The obtained linear peptide on the resin was subjected to the general method ClAc-3. Polypeptide on the resin was treated with Cleavage Cocktail-A (25 mL) to furnish the linear peptide. The crude containing the linear peptide (0.375 mmol as theoretical based on the resin used) was subjected to the peptide cyclization condition-2. The resulting residue was purified by preparative reversed-phase HPLC (Method P-C). Pure fractions were combined and lyophilized to afford the title macrocyclic peptide. HPLC-MS (Method A-A): 5.64 min, ESI-MS m/z: 877.6 [M+3H]3+, AUC (UV 225 nm): 92%.

### Example B10: PDC_EphA2-00007196-C010 (SEQ ID NOS 279, 279, 231, 231 and 231, Respectively, in Order of Appearance)

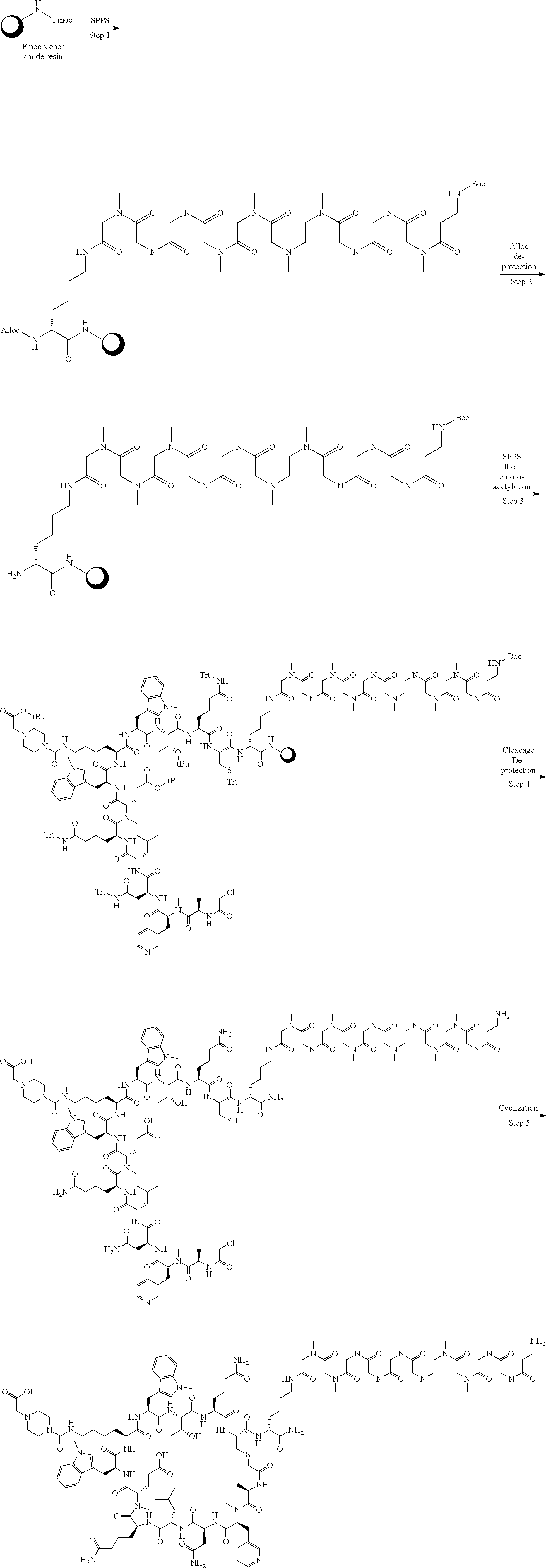

Intermediates on-resin were synthesized in the analogous manner to the step 1 of the scheme A. The intermediate peptide on-resin was synthesized on Fmoc-Sieber amide Resin (0.25 mmol) in the analogous manner to the step 1 of the scheme A. The resin was shaken with Pd(PPh3)4 (0.2 eq) and PhSiH3 (10 eq) at room temperature for 1 h. The peptide sequence was then synthesized on the intermediate following general peptide synthesis method A. The obtained linear peptide on the resin was subjected to the general method ClAc-3. Polypeptide on the resin was treated with Cleavage Cocktail-A (18 mL) to furnish the linear peptide. The crude containing the linear peptide (0.375 mmol as theoretical based on the resin used) was subjected to the peptide cyclization condition-2. The resulting residue was purified by preparative reversed-phase HPLC (Method P-C). Pure fractions were combined and lyophilized to afford the title macrocyclic peptide. HPLC-MS (Method A-A): 5.58 min, ESI-MS m/z: 920.3 [M+3H]3+, AUC (UV 225 nm): 96%.

### Example B11

The following Examples were prepared by following similar methods described in the examples above. The amino acid sequence for each peptide was shown in Table B6 and B6-1.

TABLE B9

| Example | | |
|---|---|---|
| No. | Structure | Analytics |
| PDC_EphA2- 00001417- C004 (SEQ ID NO: 223) | 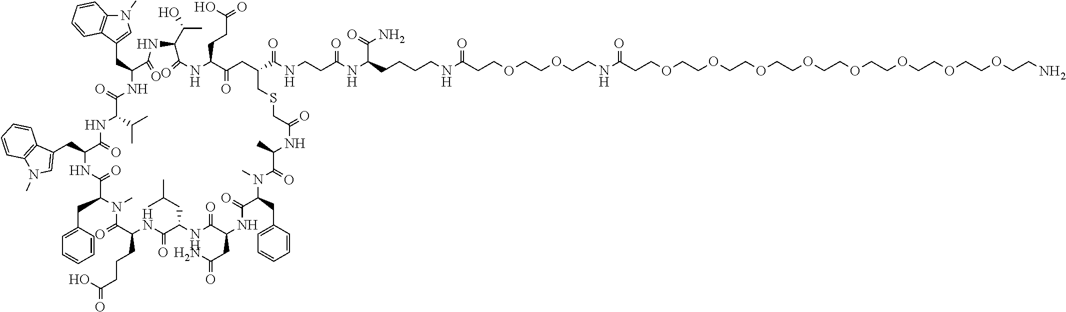 | HPLC-MS (Method A- B): 5.90 min ESI-MS m/z: 812.9 [M + 3H]3+ |
| PDC_EphA2- 00008093- C004 (SEQ ID NO: 225) | 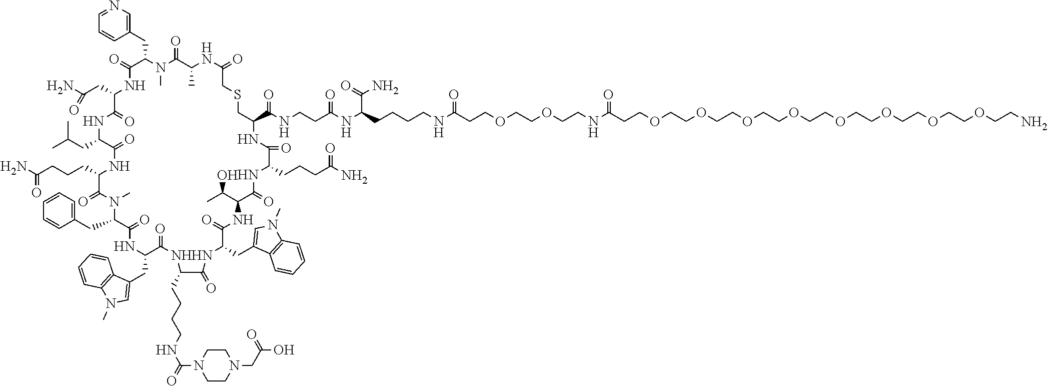 | HPLC-MS (Method A- B): 3.39 min ESI-MS m/z: 883.6 [M + 3H]3+ |
| PDC_EphA2- 00008094- C004 (SEQ ID NO: 226) | 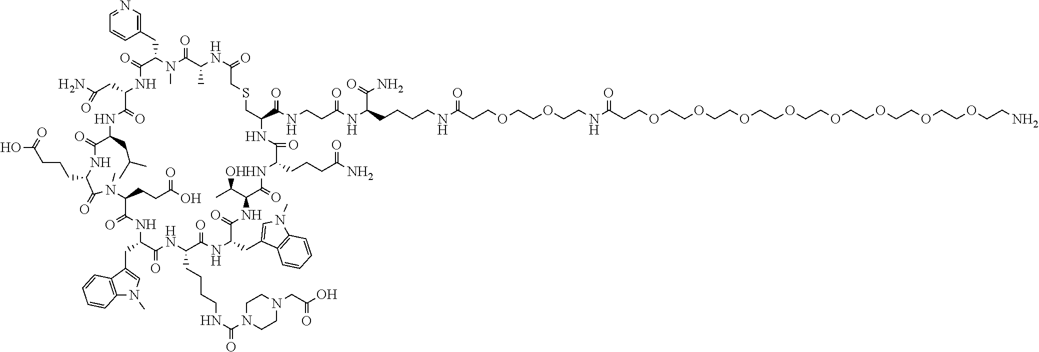 | HPLC-MS (Method A- A): 5.56 min ESI-MS m/z: 878.0 [M + 3H]3+ |
| PDC_EphA2- 00010011- C004 (SEQ ID NO: 227) | 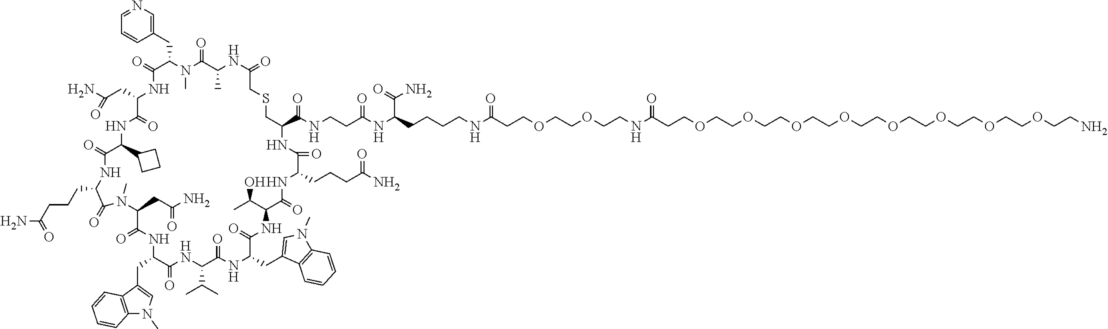 | HPLC-MS (Method A- B): 3.04 min ESI-MS m/z: 805.7 [M + 3H]3+ |
| PDC_EphA2- 00010012- C004 (SEQ ID NO: 228) | 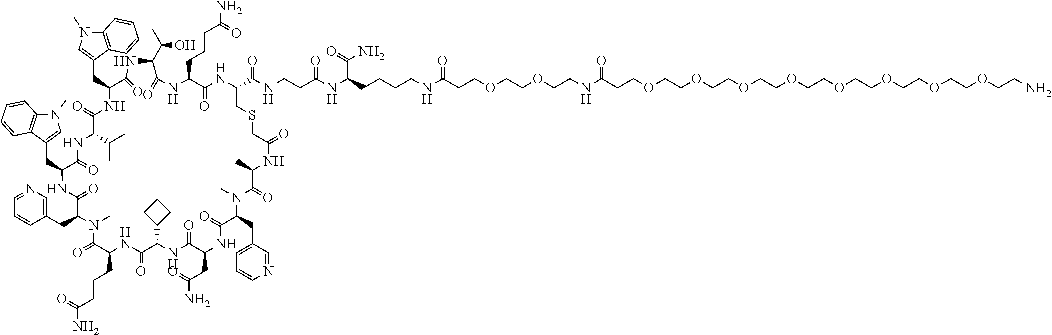 | HPLC-MS (Method A- B): 2.79 min ESI-MS m/z: 816.9 [M + 3H]3+ |
| PDC_EphA2- 00013693- C004 (SEQ ID NO: 229) | 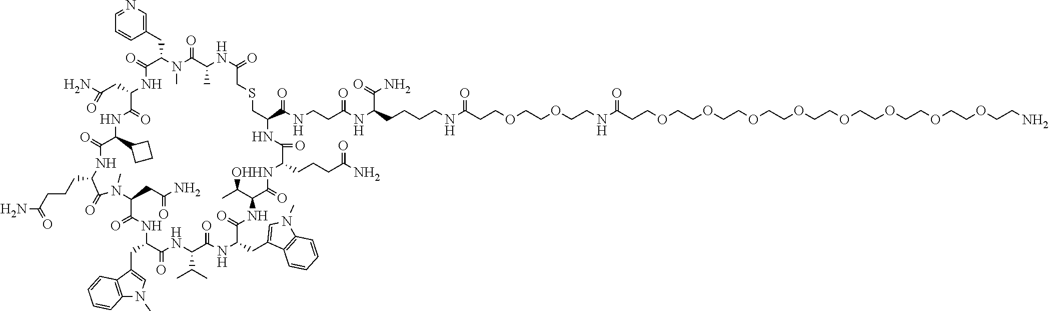 | HPLC-MS (Method A- B): 4.47 min ESI-MS m/z: 805.1 [M + 3H]3+ |
| PDC_EphA2- 00008093- C010 (SEQ ID NO: 230) | 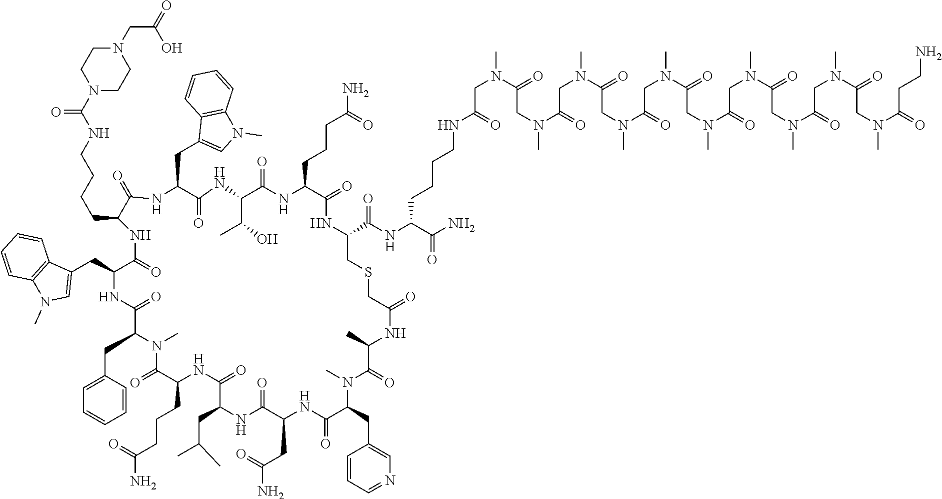 | HPLC-MS (Method A- B): 3.12 min ESI-MS m/z: 926.3 [M + 3H]3+ |

### Example B12: PDC_EphA2-00026603-C001 (SEQ ID No: 241)

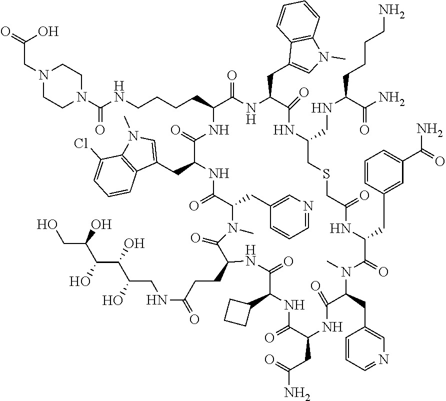 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](CN[C@@H](CCCCN)C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1Cc1cccnc1]

The peptide sequence was synthesized on Fmoc-Sieber amide Resin (0.250 mmol) following general peptide synthesis method A. The obtained peptide on the resin was subjected to the general method ClAc-3. The polypeptide on the resin was treated with Cleavage Cocktail-A (10 mL) to furnish the linear peptide. The crude containing the linear peptide (0.250 mmol as theoretical based on the resin used) was subjected to the peptide cyclization condition-2. The resulting residue was purified by preparative reversed-phase HPLC (Method P-A). Pure fractions were combined and lyophilized to afford the title macrocyclic peptide. HPLC-MS (Method A-A): 5.13 min, ESI-MS m/z: 685.5 [M+3H]3+, AUC (UV 225 nm): 98%.

### Example B13

The following Examples were prepared by following similar methods described in the examples above. The amino acid sequence for each peptide was shown in Table B6 and B6-1.

TABLE B10

| Example | | |
|---|---|---|
| No. | Structure | Analytics |
| PDC_EphA2- 00026600- C001 (SEQ ID NO: 238) | 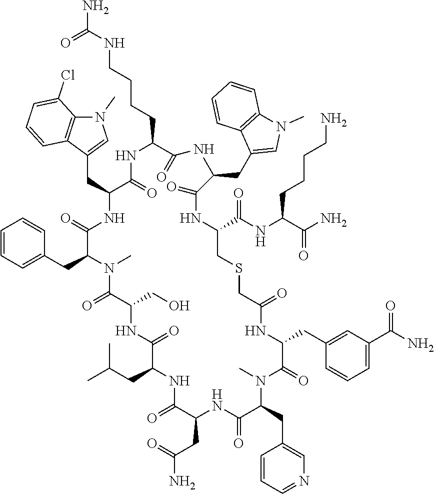 | HPLC-MS (Method A- B): 3.84 min ESI-MS m/z: 862.4 [M + 2H]2+ |
| PDC_EphA2- 00026601- C001 (SEQ ID NO: 239) | 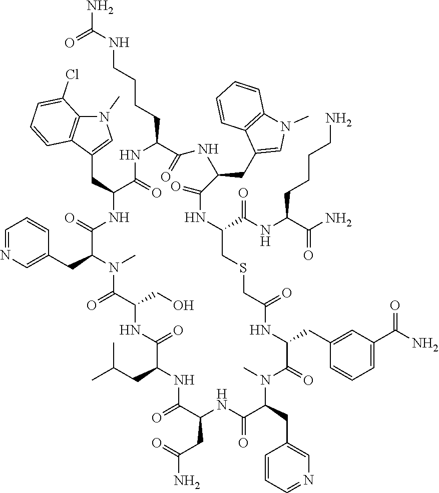 | HPLC-MS (Method A- A): 5.27 min ESI-MS m/z: 863.0 [M + 2H]2+ |
| PDC_EphA2- 00026602- C001 (SEQ ID NO: 240) | 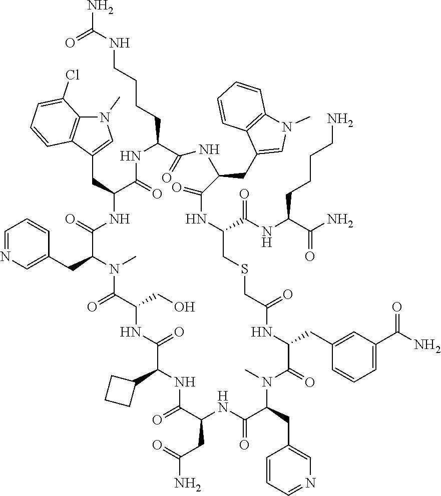 [SMILES: CN1C(=O)[C@H](CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(=O)N[C@@H](CCCCN)C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@@H]1Cc1cccnc1] | HPLC-MS (Method A- A): 5.54 min ESI-MS m/z: 861.5 [M + 2H]2+ |
| PDC_EphA2- 00026604- C001 (SEQ ID NO: 242) | 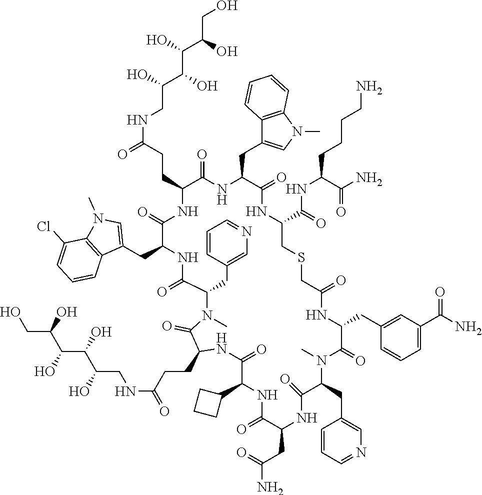 | HPLC-MS (Method A- A): 5.19 min ESI-MS m/z: 683.5 [M + 3H]3+ |
| PDC_EphA2- 00026606- C001 (SEQ ID NO: 243) | 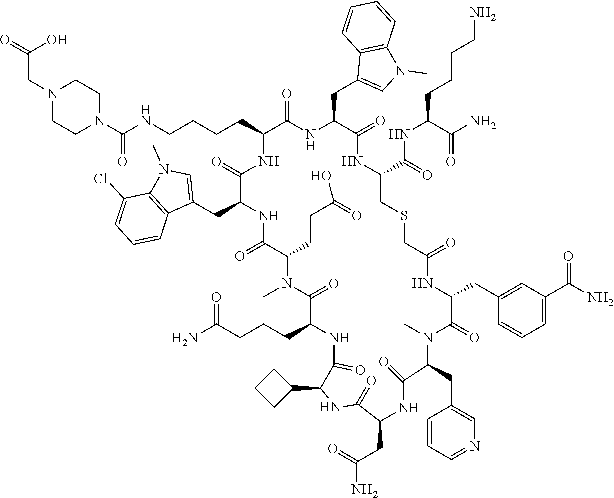 | HPLC-MS (Method A- A): 5.59 min ESI-MS m/z: 629.1 [M + 3H]3+ |
| PDC_EphA2- 00026607- C001 (SEQ ID NO: 244) | 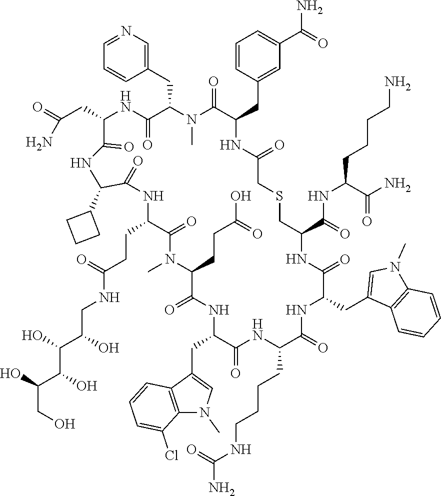 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(=O)N[C@@H](CCCCN)C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@@H]1CCC(=O)O] | HPLC-MS (Method A- A): 5.66 min ESI-MS m/z: 954.6 [M + 2H]2+ |
| PDC_EphA2- 00026608- C001 (SEQ ID NO: 245) | 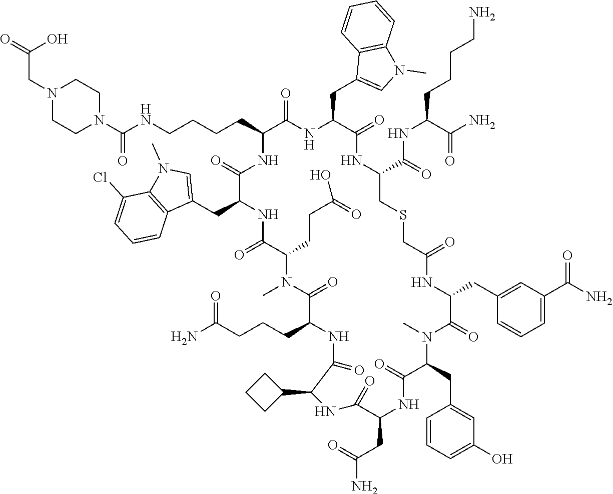 | HPLC-MS (Method A- B): 4.00 min ESI-MS m/z: 950.7 [M + 2H]2+ |

### Example B14

The following Examples were prepared by following similar methods described in the examples above. The amino acid sequence for each peptide was shown in Table B6 and B6-1.

TABLE B11

| Example | | |
|---|---|---|
| No. | Structure | Analytics |
| PDC_EphA2- 00020708 (SEQ ID NO: 150) | 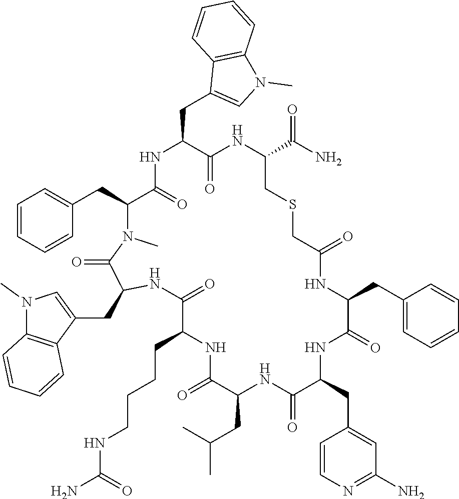 [SMILES: CC(C)C[C@@H]1NC(=O)[C@H](Cc2ccnc(N)c2)NC(=O)[C@H](Cc2ccccc2)NC(=O)CSC[C@@H](C(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)[C@H](CCCCNC(N)=O)NC1=O] | HPLC-MS (Method A-B): 5.05 min ESI-MS m/z: 659.1 [M + 2H]2+ |
| PDC_EphA2- 00022601 (SEQ ID NO: 246) | 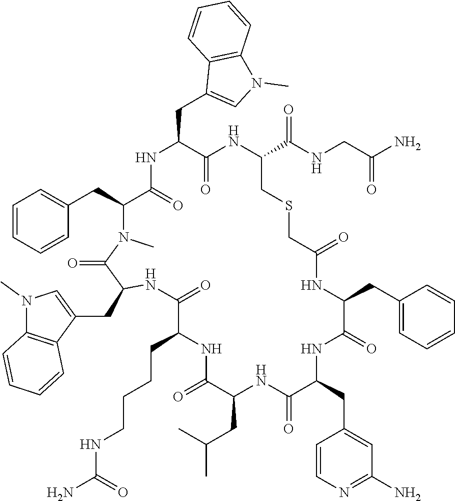 [SMILES: CC(C)C[C@@H]1NC(=O)[C@H](Cc2ccnc(N)c2)NC(=O)[C@H](Cc2ccccc2)NC(=O)CSC[C@@H](C(=O)NCC(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)[C@H](CCCCNC(N)=O)NC1=O] | HPLC-MS (Method A-B): 4.85 min ESI-MS m/z: 687.7 [M + 2H]2+ |
| PDC_EphA2- 00022602 (SEQ ID NO: 247) | 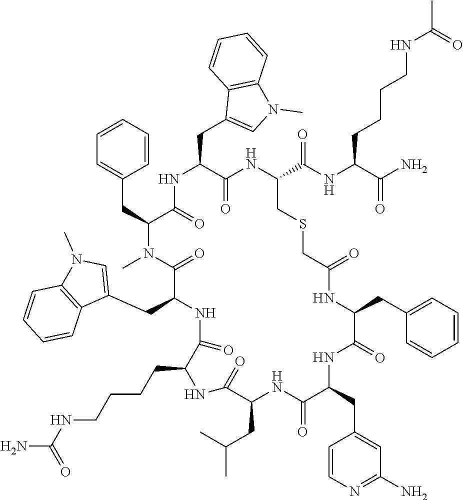 [SMILES: CC(=O)NCCCC[C@H](NC(=O)[C@@H]1CSCC(=O)N[C@@H](Cc2ccccc2)C(=O)N[C@@H](Cc2ccnc(N)c2)C(=O)N[C@@H](CC(C)C)C(=O)N[C@@H](CCCCNC(N)=O)C(=O)N[C@@H](Cc2cn(C)c3ccccc23)C(=O)N(C)[C@@H](Cc2ccccc2)C(=O)N[C@@H](Cc2cn(C)c3ccccc23)C(=O)N1)C(N)=O] | HPLC-MS (Method A-B): 4.85 min ESI-MS m/z: 744.2 [M + 2H]2+ |
| PDC_EphA2- 00022603 (SEQ ID NO: 248) | 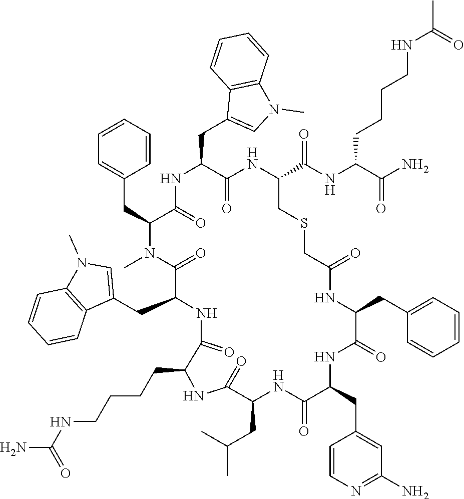 [SMILES: CC(=O)NCCCC[C@@H](NC(=O)[C@@H]1CSCC(=O)N[C@@H](Cc2ccccc2)C(=O)N[C@@H](Cc2ccnc(N)c2)C(=O)N[C@@H](CC(C)C)C(=O)N[C@@H](CCCCNC(N)=O)C(=O)N[C@@H](Cc2cn(C)c3ccccc23)C(=O)N(C)[C@@H](Cc2ccccc2)C(=O)N[C@@H](Cc2cn(C)c3ccccc23)C(=O)N1)C(N)=O] | HPLC-MS (Method A-B): 4.84 min ESI-MS m/z: 744.2 [M + 2H]2+ |
| PDC_EphA2- 00019498- L092 (SEQ ID NO: 251) | 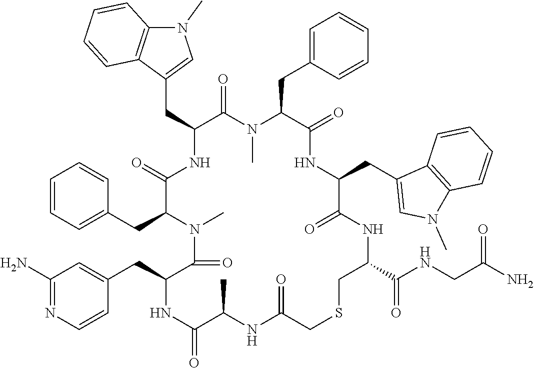 [SMILES: C[C@H]1NC(=O)CSC[C@@H](C(=O)NCC(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)[C@H](Cc2ccccc2)N(C)C(=O)[C@H](Cc2ccnc(N)c2)NC1=O] | HPLC-MS (Method A-B): 5.46 min ESI-MS m/z: 588.1 [M + 2H]2+ |
| PDC_EphA2- 00020295- L092 (SEQ ID NO: 249) | 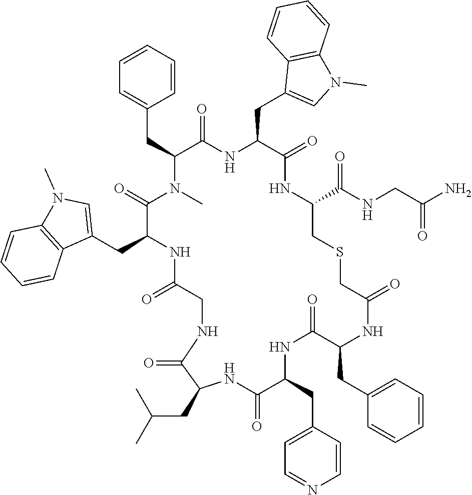 [SMILES: CC(C)C[C@@H]1NC(=O)[C@H](Cc2ccncc2)NC(=O)[C@H](Cc2ccccc2)NC(=O)CSC[C@@H](C(=O)NCC(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)CNC1=O] | HPLC-MS (Method A-B): 5.31 min ESI-MS m/z: 623.1 [M + 2H]2+ |
| PDC_EphA2- 00026479 (SEQ ID NO: 151) | 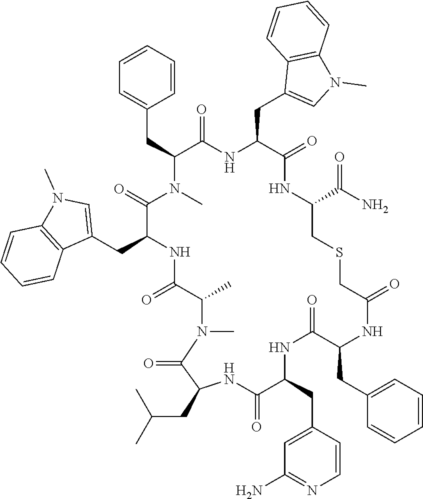 [SMILES: CC(C)C[C@@H]1NC(=O)[C@H](Cc2ccnc(N)c2)NC(=O)[C@H](Cc2ccccc2)NC(=O)CSC[C@@H](C(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)[C@H](C)N(C)C1=O] | HPLC-MS (Method A-B): 6.04 min ESI-MS m/z: 616.2 [M + 2H]2+ |
| PDC_EphA2- 00026481 (SEQ ID NO: 152) | 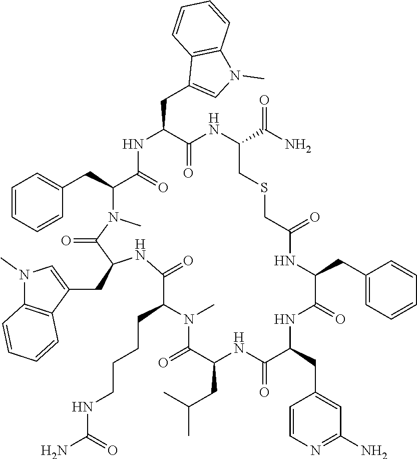 [SMILES: CC(C)C[C@@H]1NC(=O)[C@H](Cc2ccnc(N)c2)NC(=O)[C@H](Cc2ccccc2)NC(=O)CSC[C@@H](C(N)=O)NC(=O)[C@H](Cc2cn(C)c3ccccc23)NC(=O)[C@H](Cc2ccccc2)N(C)C(=O)[C@H](Cc2cn(C)c3ccccc23)NC(=O)[C@H](CCCCNC(N)=O)N(C)C1=O] | HPLC-MS (Method A-B): 5.32 min ESI-MS m/z: 666.2 [M + 2H]2+ |
| PDC_EphA2- 00021407- L092 (SEQ ID NO: 235) | 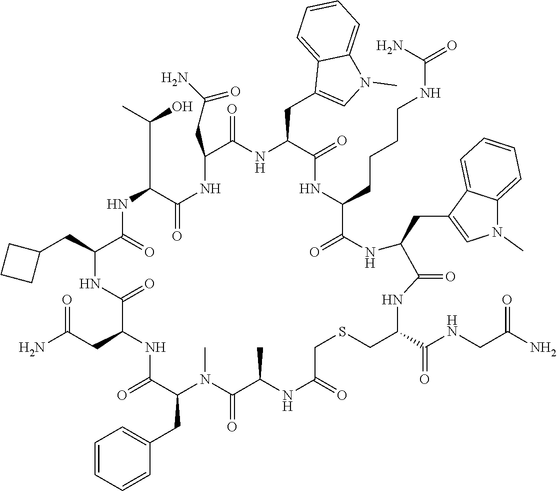 [SMILES: C[C@H]1NC(=O)CSC[C@@H](C(=O)NCC(N)=O)NC(=O)[C@H](Cc2cn(C)c3ccccc23)NC(=O)[C@H](CCCCNC(N)=O)NC(=O)[C@H](Cc2cn(C)c3ccccc23)NC(=O)[C@H](CC(N)=O)NC(=O)[C@H]([C@@H](C)O)NC(=O)[C@H](CC2CCC2)NC(=O)[C@H](CC(N)=O)NC(=O)[C@H](Cc2ccccc2)N(C)C1=O] | |
| PDC_EphA2- 00022057- L092 (SEQ ID NO: 236) | 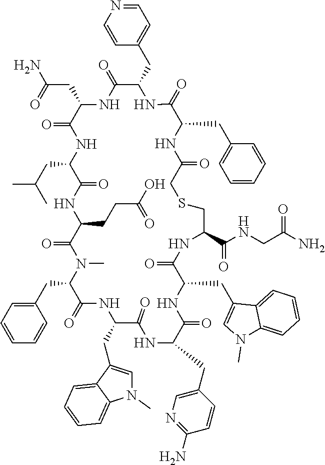 [SMILES: CC(C)C[C@@H]1NC(=O)[C@H](CC(N)=O)NC(=O)[C@H](Cc2ccncc2)NC(=O)[C@H](Cc2ccccc2)NC(=O)CSC[C@@H](C(=O)NCC(N)=O)NC(=O)[C@H](Cc2cn(C)c3ccccc23)NC(=O)[C@H](Cc2ccc(N)nc2)NC(=O)[C@H](Cc2cn(C)c3ccccc23)NC(=O)[C@H](Cc2ccccc2)N(C)C(=O)[C@H](CCC(=O)O)NC1=O] | |
| PDC_EphA2- 00022417 (SEQ ID NO: 123) | 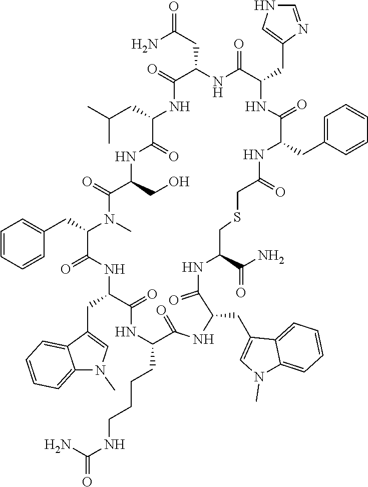 [SMILES: CC(C)C[C@@H]1NC(=O)[C@H](CC(N)=O)NC(=O)[C@H](Cc2c[nH]cn2)NC(=O)[C@H](Cc2ccccc2)NC(=O)CSC[C@@H](C(N)=O)NC(=O)[C@H](Cc2cn(C)c3ccccc23)NC(=O)[C@H](CCCCNC(N)=O)NC(=O)[C@H](Cc2cn(C)c3ccccc23)NC(=O)[C@H](Cc2ccccc2)N(C)C(=O)[C@H](CO)NC1=O] | |
| PDC_EphA2- 00022606 (SEQ ID NO: 232) | 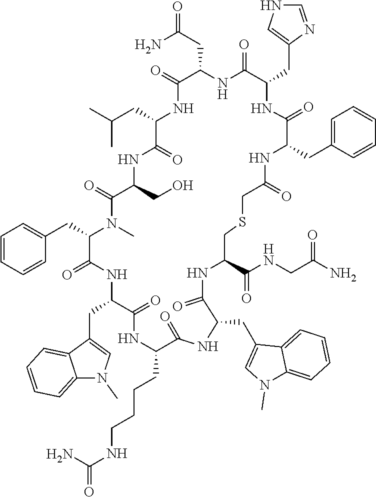 [SMILES: CC(C)C[C@@H]1NC(=O)[C@H](CC(N)=O)NC(=O)[C@H](Cc2c[nH]cn2)NC(=O)[C@H](Cc2ccccc2)NC(=O)CSC[C@@H](C(=O)NCC(N)=O)NC(=O)[C@H](Cc2cn(C)c3ccccc23)NC(=O)[C@H](CCCCNC(N)=O)NC(=O)[C@H](Cc2cn(C)c3ccccc23)NC(=O)[C@H](Cc2ccccc2)N(C)C(=O)[C@H](CO)NC1=O] | |
| PDC_EphA2- 00022607 (SEQ ID NO: 233) | 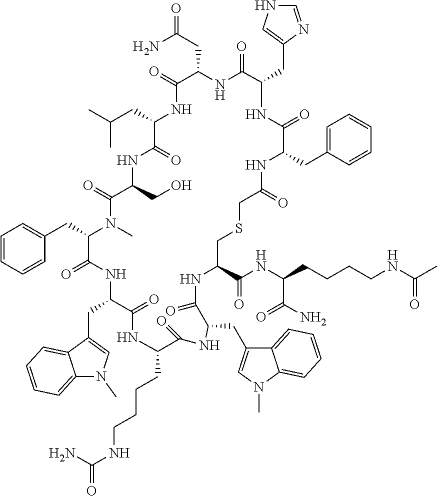 [SMILES: CC(=O)NCCCC[C@H](NC(=O)[C@@H]1CSCC(=O)N[C@@H](Cc2ccccc2)C(=O)N[C@@H](Cc2c[nH]cn2)C(=O)N[C@@H](CC(N)=O)C(=O)N[C@@H](CC(C)C)C(=O)N[C@@H](CO)C(=O)N(C)[C@@H](Cc2ccccc2)C(=O)N[C@@H](Cc2cn(C)c3ccccc23)C(=O)N[C@@H](CCCCNC(N)=O)C(=O)N[C@@H](Cc2cn(C)c3ccccc23)C(=O)N1)C(N)=O] | |
| PDC_EphA2- 00022608 (SEQ ID NO: 234) | 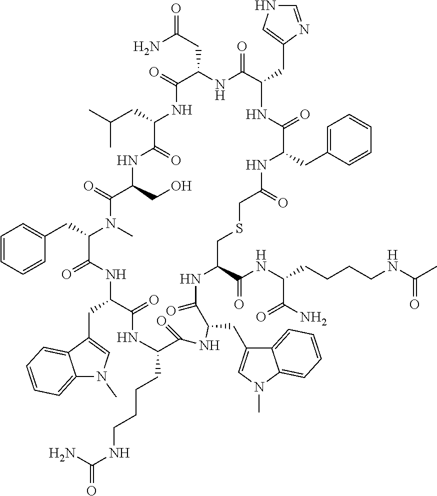 [SMILES: CC(=O)NCCCC[C@@H](NC(=O)[C@@H]1CSCC(=O)N[C@@H](Cc2ccccc2)C(=O)N[C@@H](Cc2c[nH]cn2)C(=O)N[C@@H](CC(N)=O)C(=O)N[C@@H](CC(C)C)C(=O)N[C@@H](CO)C(=O)N(C)[C@@H](Cc2ccccc2)C(=O)N[C@@H](Cc2cn(C)c3ccccc23)C(=O)N[C@@H](CCCCNC(N)=O)C(=O)N[C@@H](Cc2cn(C)c3ccccc23)C(=O)N1)C(N)=O] | |
| PDC_EphA2- 00026600 (SEQ ID NO: 124) | 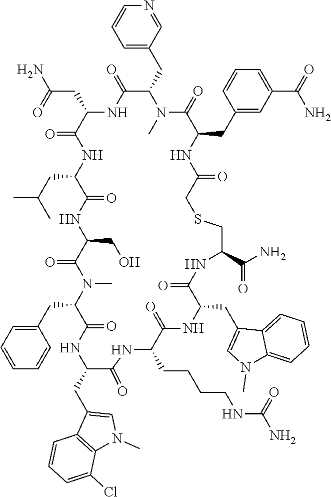 [SMILES: CC(C)C[C@@H]1NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@H](Cc2ccccc2)N(C)C(=O)[C@H](CO)NC1=O] | |
| PDC_EphA2- 00026601 (SEQ ID NO: 125) | 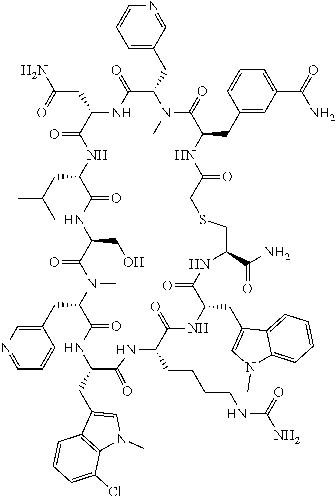 [SMILES: CC(C)C[C@@H]1NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@H](Cc2cccnc2)N(C)C(=O)[C@H](CO)NC1=O] | |
| PDC_EphA2- 00026602 (SEQ ID NO: 126 | 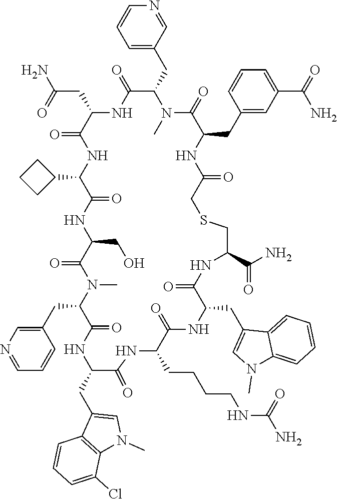 [SMILES: CN1C(=O)[C@H](CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@@H]1Cc1cccnc1] | |
| PDC_EphA2- 00026603 (SEQ ID NO: 127) | 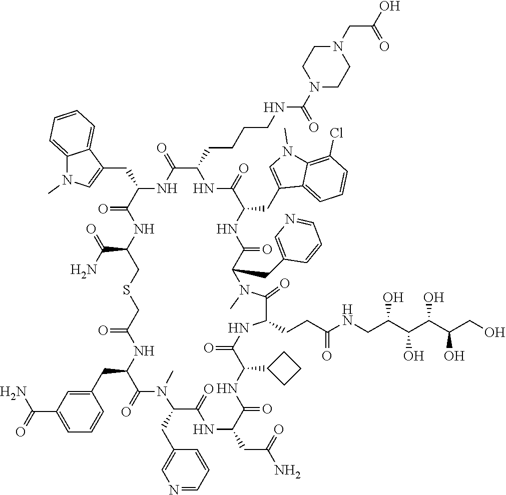 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1Cc1cccnc1] | |
| PDC_EphA2- 00026604 (SEQ ID NO: 128) | 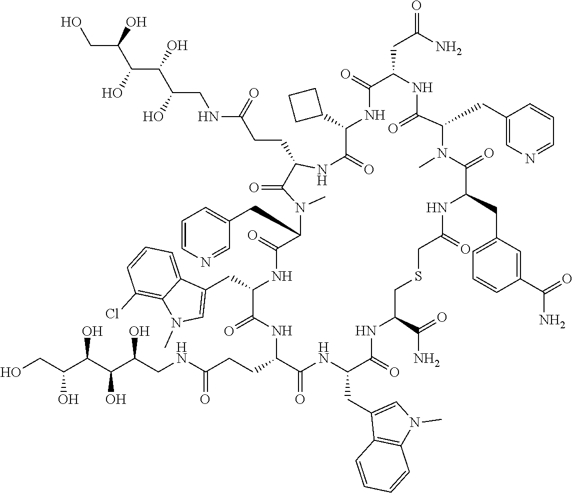 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3c(Cl)cccc23)NC(=O)[C@@H]1Cc1cccnc1] | |
| PDC_EphA2- 00026605 (SEQ ID NO: 129) | 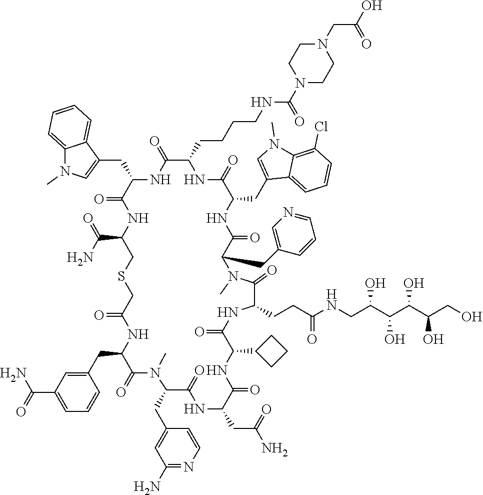 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2ccnc(N)c2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1Cc1cccnc1] | |
| PDC_EphA2- 00026606 (SEQ ID NO: 130) | 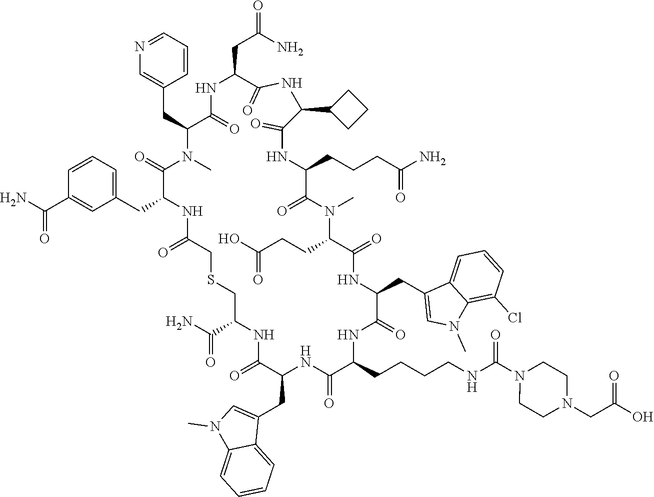 [SMILES: CN1C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1CCC(=O)O] | |
| PDC_EphA2- 00026607 (SEQ ID NO: 131) | 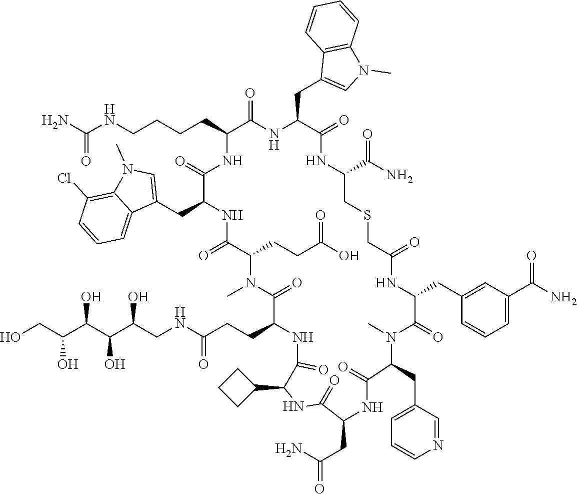 [SMILES: CN1C(=O)[C@H](CCC(=O)NC[C@H](O)[C@@H](O)[C@H](O)[C@H](O)CO)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(N)=O)NC(=O)[C@H](Cc2cn(C)c3c(Cl)cccc23)NC(=O)[C@@H]1CCC(=O)O] | |
| PDC_EphA2- 00026608 (SEQ ID NO: 132) | 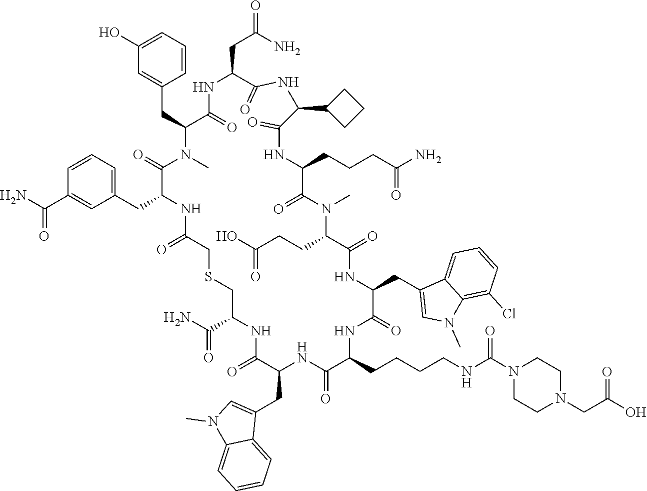 [SMILES: CN1C(=O)[C@H](CCCC(N)=O)NC(=O)[C@H](C2CCC2)NC(=O)[C@H](CC(N)=O)NC(=O)[C@H](Cc2cccc(O)c2)N(C)C(=O)[C@@H](Cc2cccc(C(N)=O)c2)NC(=O)CSC[C@@H](C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1CCC(=O)O] | |
| PDC_EphA2- 00026609 (SEQ ID NO: 133) | 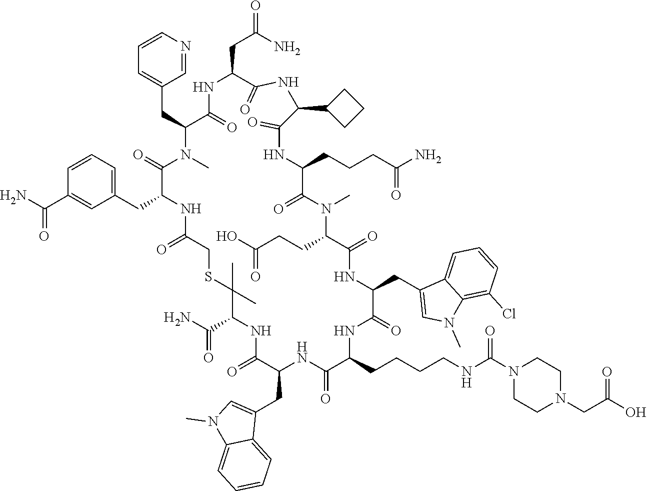 [SMILES: CN1C(=O)[C@H](CCCC(N)=O)NC(=O)[C@H](C2CCC2)NC(=O)[C@H](CC(N)=O)NC(=O)[C@H](Cc2cccnc2)N(C)C(=O)[C@@H](Cc2cccc(C(N)=O)c2)NC(=O)CSC(C)(C)[C@@H](C(N)=O)NC(=O)[C@H](Cc2cn(C)c3ccccc23)NC(=O)[C@H](CCCCNC(=O)N2CCN(CC(=O)O)CC2)NC(=O)[C@H](Cc2cn(C)c3c(Cl)cccc23)NC(=O)[C@@H]1CCC(=O)O] | |
| PDC_EphA2- 00026610 (SEQ ID NO: 134) | 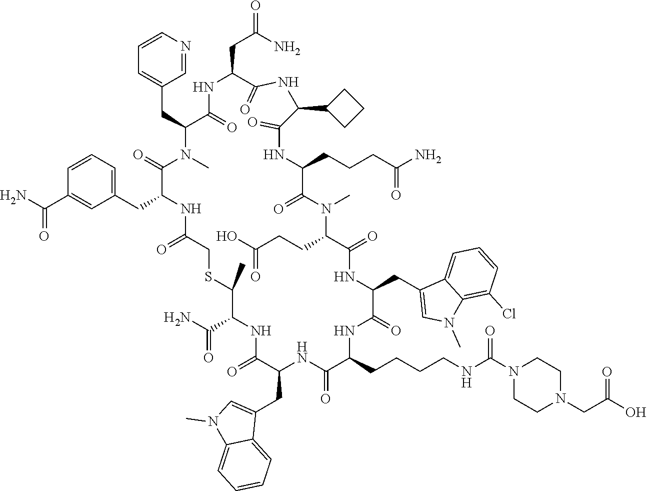 [SMILES: C[C@@H]1SCC(=O)N[C@H](Cc2cccc(C(N)=O)c2)C(=O)N(C)[C@@H](Cc2cccnc2)C(=O)N[C@@H](CC(N)=O)C(=O)N[C@@H](C2CCC2)C(=O)N[C@@H](CCCC(N)=O)C(=O)N(C)[C@@H](CCC(=O)O)C(=O)N[C@@H](Cc2cn(C)c3c(Cl)cccc23)C(=O)N[C@@H](CCCCNC(=O)N2CCN(CC(=O)O)CC2)C(=O)N[C@@H](Cc2cn(C)c3ccccc23)C(=O)N[C@@H]1C(N)=O] | |
| PDC_EphA2- 00026611 (SEQ ID NO: 135) | 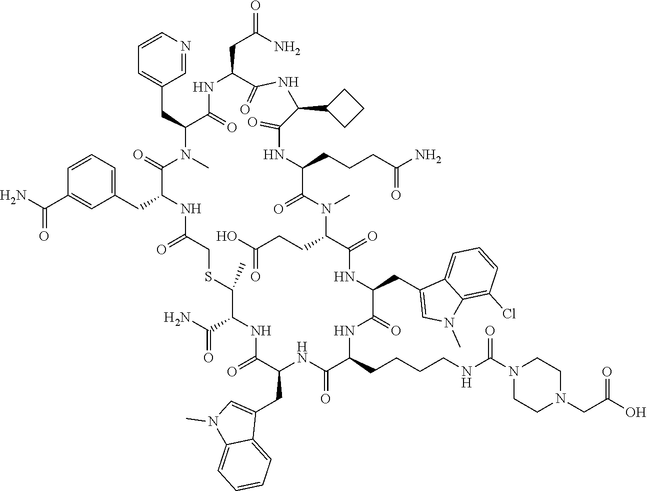 [SMILES: C[C@H]1SCC(=O)N[C@H](Cc2cccc(C(N)=O)c2)C(=O)N(C)[C@@H](Cc2cccnc2)C(=O)N[C@@H](CC(N)=O)C(=O)N[C@@H](C2CCC2)C(=O)N[C@@H](CCCC(N)=O)C(=O)N(C)[C@@H](CCC(=O)O)C(=O)N[C@@H](Cc2cn(C)c3c(Cl)cccc23)C(=O)N[C@@H](CCCCNC(=O)N2CCN(CC(=O)O)CC2)C(=O)N[C@@H](Cc2cn(C)c3ccccc23)C(=O)N[C@@H]1C(N)=O] | |

### Example B15. Interaction Analysis by SPR was Performed Using Biacore (Cytiva)

SPR assay was performed using Biacore T200 (Cytiva).

The HBS-P+ buffer (10 mM HEPES (pH7.4), 150 mM NaCl, 0.05% (v/v) Surfactant P20) containing 1% DMSO was used as running buffer. Recombinant Human EphA2 Fc Protein (Fc tag, Elabscience) was captured by Human Antibody Capture kit (Cytiva). Peptide samples in DMSO were diluted with running buffer, and prepared five serial dilutions. Using these serial dilutions, kinetics of peptides against EphA2 was measured at a flow rate of 30 mL/min at 25 °C. The method adopted for sample measurement was a single-cycle kinetics method. The analysis was conducted using the evaluation software 3.0 provided with Biacore T200. Kinetics fitting was done on the difference data obtained by subtracting the baseline data from sample measurement data. KD values were calculated based on the association rate constant (ka) and dissociation rate constant (kd).

TABLE B12

| | SPR |
|---|---|
| Peptide ID | Kd (nM) |
| PDC_EphA2-00001417 | B |
| PDC_EphA2-00008082 | A |
| PDC_EphA2-00008083 | A |
| PDC_EphA2-00008084 | B |
| PDC_EphA2-00008085 | B |
| PDC_EphA2-00008086 | A |
| PDC_EphA2-00008087 | A |
| PDC_EphA2-00008088 | A |
| PDC_EphA2-00008089 | A |
| PDC_EphA2-00008090 | A |
| PDC_EphA2-00008091 | A |
| PDC_EphA2-00008092 | A |
| PDC_EphA2-00008093 | A |
| PDC_EphA2-00008094 | A |
| PDC_EphA2-00008095 | A |
| PDC_EphA2-00007196 | A |
| PDC_EphA2-00008097 | B |
| PDC_EphA2-00009998 | A |
| PDC_EphA2-00009999 | A |
| PDC_EphA2-00010000 | A |
| PDC_EphA2-00010001 | A |
| PDC_EphA2-00010002 | A |
| PDC_EphA2-00010003 | A |
| PDC_EphA2-00010004 | A |
| PDC_EphA2-00010005 | A |
| PDC_EphA2-00010006 | A |
| PDC_EphA2-00010007 | A |
| PDC_EphA2-00010008 | A |
| PDC_EphA2-00010009 | A |
| PDC_EphA2-00010010 | A |
| PDC_EphA2-00010011 | A |
| PDC_EphA2-00010012 | A |
| PDC_EphA2-00013693 | A |
| PDC_EphA2-00013703 | A |
| PDC_EphA2-00013704 | A |
| PDC_EphA2-00013705 | A |
| PDC_EphA2-00018526 | A |
| PDC_EphA2-00018527 | A |
| PDC_EphA2-00018528 | A |
| PDC_EphA2-00018529 | A |
| PDC_EphA2-00018530 | A |
| PDC_EphA2-00018531 | A |
| PDC_EphA2-00018532 | A |
| PDC_EphA2-00018533 | A |
| PDC_EphA2-00018534 | A |
| PDC_EphA2-00018535 | A |
| PDC_EphA2-00018536 | A |
| PDC_EphA2-00018537 | A |
| PDC_EphA2-00018538 | A |
| PDC_EphA2-00018539 | A |
| PDC_EphA2-00018540 | A |
| PDC_EphA2-00019421 | A |
| PDC_EphA2-00019422 | A |
| PDC_EphA2-00019423 | A |
| PDC_EphA2-00019424 | A |
| PDC_EphA2-00019425 | A |
| PDC_EphA2-00019426 | A |
| PDC_EphA2-00019427 | A |
| PDC_EphA2-00019428 | A |
| PDC_EphA2-00019429 | A |
| PDC_EphA2-00019430 | A |
| PDC_EphA2-00019431 | A |
| PDC_EphA2-00019432 | A |
| PDC_EphA2-00019433 | A |
| PDC_EphA2-00019434 | A |
| PDC_EphA2-00019435 | A |
| PDC_EphA2-00019436 | A |
| PDC_EphA2-00019437 | A |
| PDC_EphA2-00019438 | A |
| PDC_EphA2-00019439 | A |
| PDC_EphA2-00019440 | A |
| PDC_EphA2-00019441 | A |
| PDC_EphA2-00019442 | A |
| PDC_EphA2-00026424 | A |
| PDC_EphA2-00026425 | A |
| PDC_EphA2-00026426 | A |
| PDC_EphA2-00022417 | C |
| PDC_EphA2-00022606 | C |
| PDC_EphA2-00022607 | C |
| PDC_EphA2-00022608 | C |
| PDC_EphA2-00021407-L092 | D |
| PDC_EphA2-00022057-L092 | C |
| PDC_EphA2-00026600 | B |
| PDC_EphA2-00026601 | B |
| PDC_EphA2-00026602 | A |
| PDC_EphA2-00026603 | A |
| PDC_EphA2-00026604 | A |
| PDC_EphA2-00026605 | B |
| PDC_EphA2-00026606 | A |
| PDC_EphA2-00026607 | A |
| PDC_EphA2-00026608 | A |
| PDC_EphA2-00026609 | B |
| PDC_EphA2-00026610 | B |
| PDC_EphA2-00026611 | A |
| PDC_EphA2-00020708 | D |
| PDC_EphA2-00022601 | D |
| PDC_EphA2-00022602 | D |
| PDC_EphA2-00022603 | D |
| PDC_EphA2-00020295-L092 | D |
| PDC_EphA2-00026479 | D |
| PDC_EphA2-00026481 | D |
| PDC_EphA2-00013917 | A |

SPR (Kd (nM)): 0 < A ≤ 1; 1 < B ≤ 10; 10 < C ≤ 100; 100 < D ≤ 400

### Example B16: Peptide Conjugates

Peptide conjugates (Scheme D-II) of the present disclosure can be synthesized by following the step outlined in scheme D shown below.

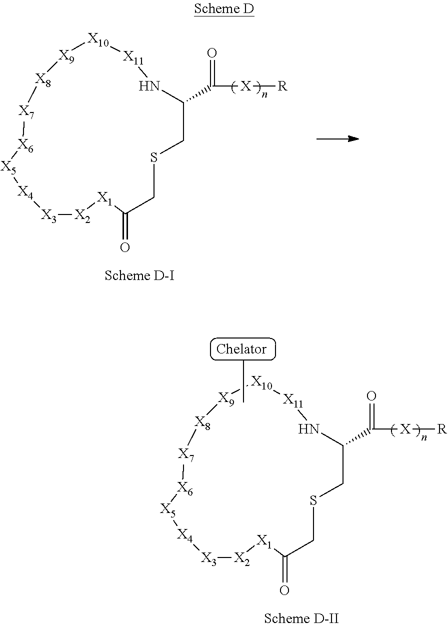

To a solution of DOTA-NHS ester (6 eq) in H₂O at 0 °C was added peptide I (1 eq) and DIPEA (9 eq) in DMF at rt. The reaction mixture was then shaken at room temperature until completion. The resulting mixture was concentrated and purified by preparative reverse phase C18-HPLC to provide the desired peptide conjugates.

Peptide conjugates (Scheme E-I) of the present disclosure can be synthesized by following the step outlined in scheme E shown below.

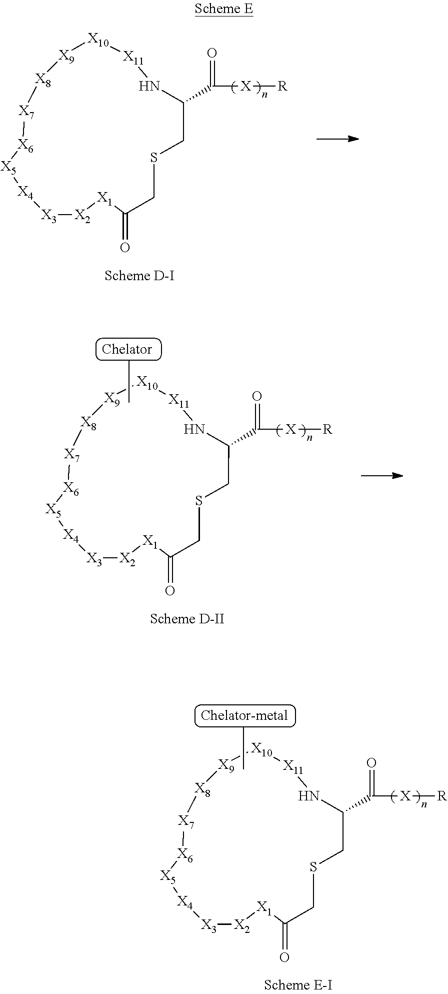

To a solution of DOTA-NHS ester (6 eq) in H₂O at 0 °C was added peptide I (1 eq) and DIPEA (9 eq) in DMF at rt. The reaction mixture was then shaken at room temperature until completion. The resulting mixture was diluted by 50 mM ammonium acetate buffer (pH5). To a solution of polypeptide IV was added 50 mM lutetium (III) chloride hexahydrate (11 eq) in 50 mM ammonium acetate buffer (pH5). The mixture was stirred at 90 °C for 1 h, and then concentrated. The resulting mixture was purified by preparative reverse phase C18-HPLC to provide the desired peptide conjugates Scheme E-I.

### General Scheme F

Macrocyclic peptides Scheme G-I in the present disclosure can be synthesized in the analogous manner to the scheme A.

Synthesis may be completed using commercially available products as is for all raw materials, building blocks, reagents, acids, bases, solid-phase resins, and solvents used in chemical synthesis in the following examples, or by a person having ordinary skill in the art using organic chemistry techniques. Note that commercial products were used for amino acids containing protecting groups unless otherwise specified.

### General Scheme G

Macrocyclic peptides Scheme G-II in the present disclosure can be synthesized in the analogous manner to the scheme D. The general synthetic scheme is described in Scheme G shown below.

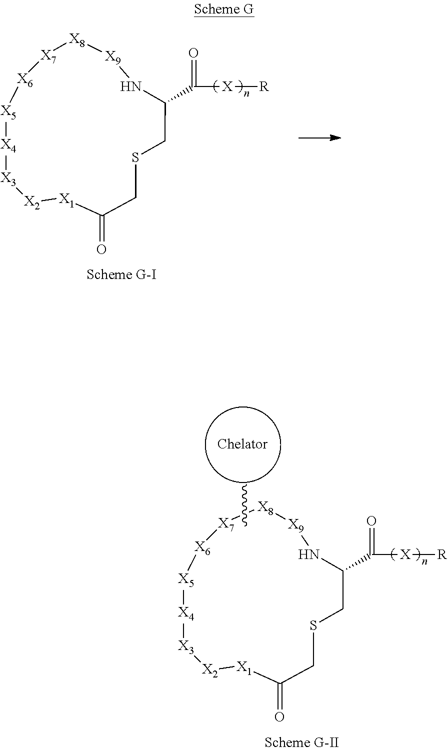

### General Scheme H

Peptide conjugates Scheme H-I in the present disclosure can be synthesized in the analogous manner to the scheme E. The general synthetic scheme is described in Scheme H shown below.

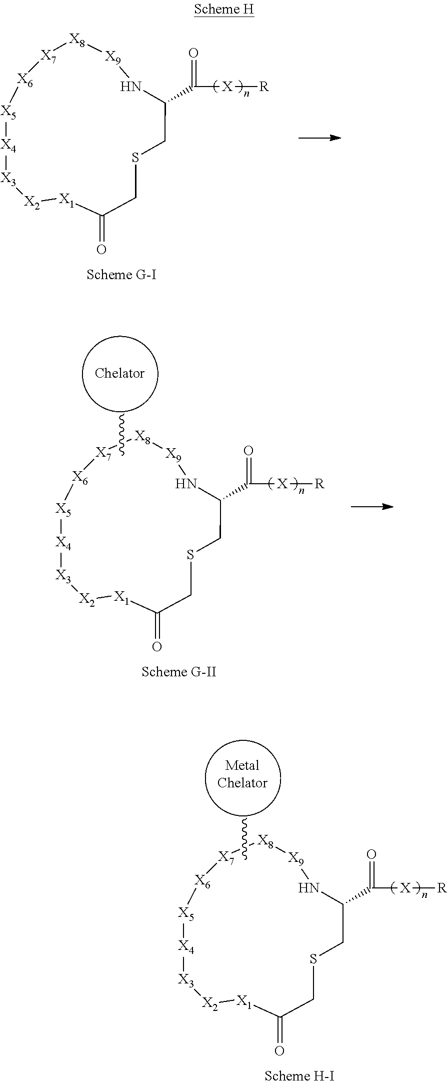

### Example B17: PDC_EphA2-00007196-C202 (SEQ ID NOS 200 and 375, Respectively, in Order of Appearance)

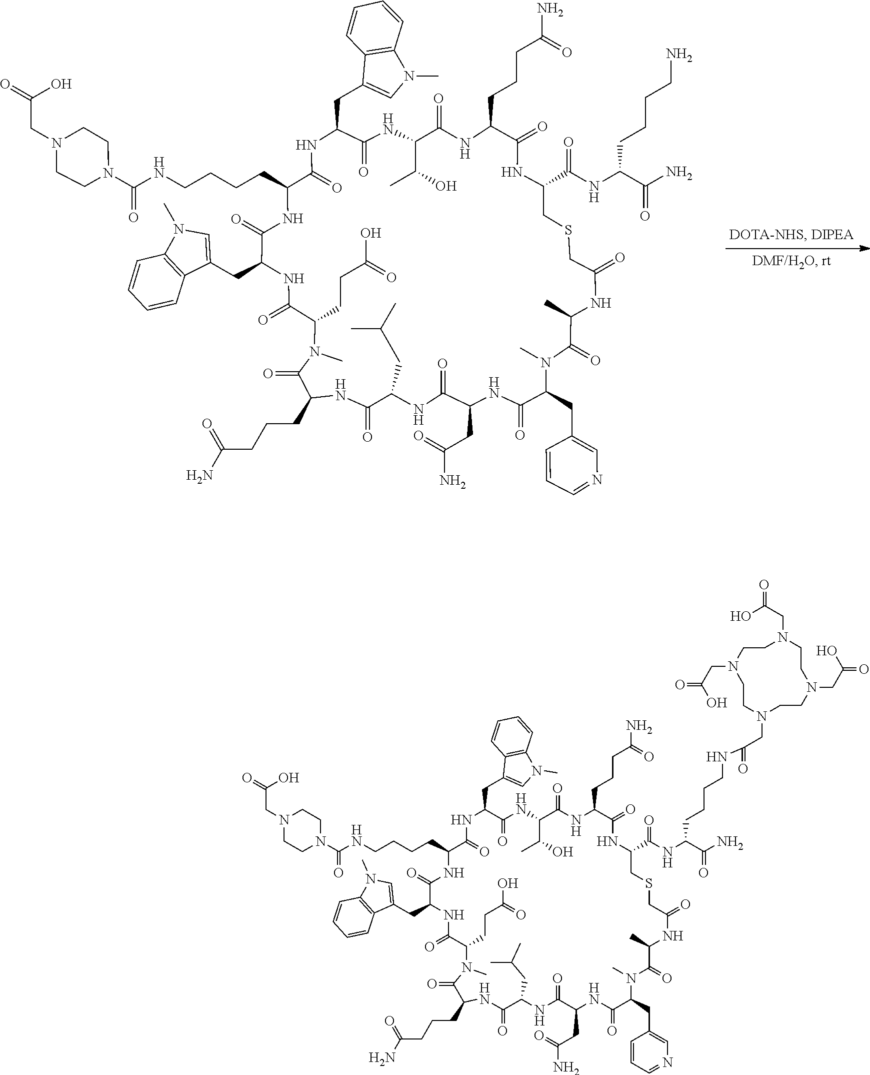

To a solution of Example-B2 (PDC_EphA2-00007196-C002) (30 mg, 0.0130 mmol) in DMF (647 μL) at 0 °C were added DIPEA (20.34 μL, 0.116 mmol) and 0.12 M aq. DOTA-NHS ester (647 μL, 0.078 mmol). The mixture was stirred at rt for 0.5 h. Then to the mixture was added DIPEA (20.34 μL, 0.116 mmol) and stirred at rt for 0.5 h. The resulting residue was purified by preparative reverse phase C18-HPLC (Method P-A). Pure fractions were combined and lyophilized to afford the title compound. HPLC-MS (Method A-A): 5.64 min, ESI-MS m/z: 788.5 [M+3H]3+, UV-area (TIC): 98%.

### Example B18

The following Examples were prepared by following similar methods described in the examples above. The amino acid sequence for each peptide was shown in Table B6 and Table B6-1. The term “Term” means the functional group at C-terminus of the peptide.

TABLE B13

| Example No. | Structure | Analytics |
|---|---|---|
| PDC_EphA2- 00008093- C202 (SEQ ID NO: 373) | 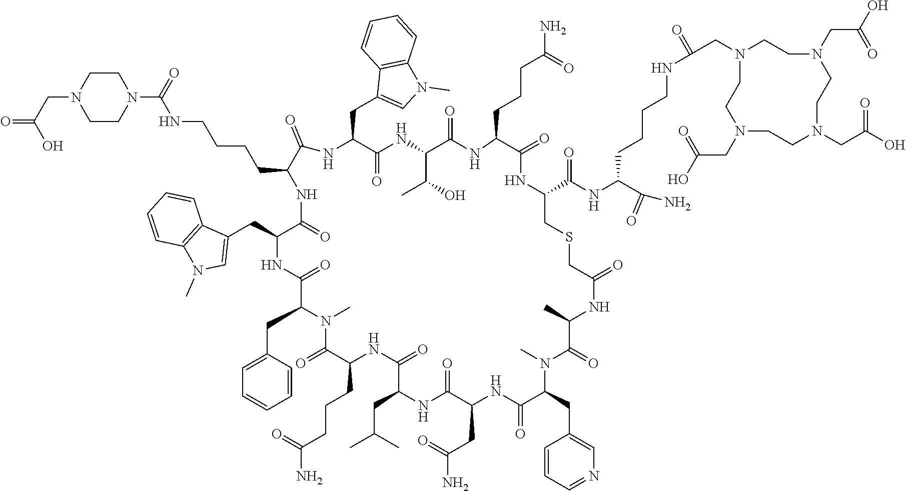 | HPLC-MS (Method A- B): 3.21 min ESI-MS m/z: 794.4 [M + 3H]3+ |
| PDC_EphA2- 00010011- C202 (SEQ ID NO: 377) | 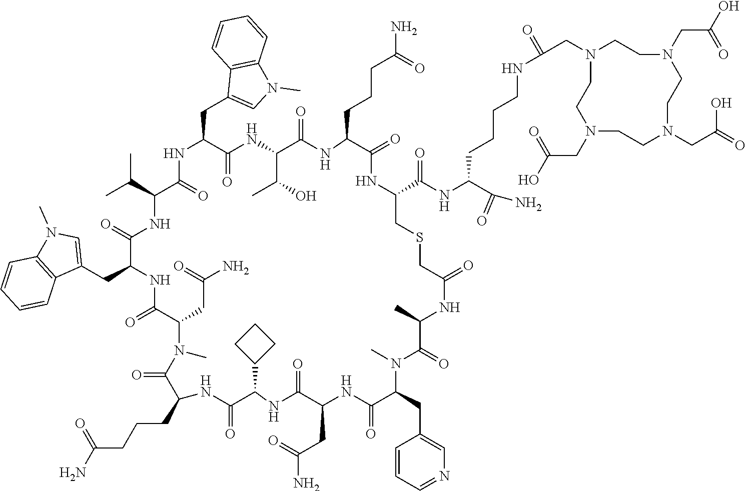 | HPLC-MS (Method A- B): 2.64 min ESI-MS m/z: 740.5 [M + 3H]3+ |
| PDC_EphA2- 00010012- C202 (SEQ ID NO: 378) | 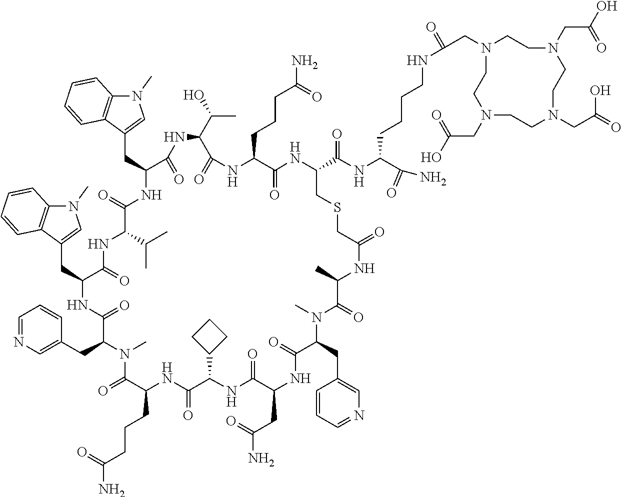 | HPLC-MS (Method A- A): 5.31 min ESI-MS m/z: 751.5 [M + 3H]3+ |
| PDC_EphA2- 00013693- C202 (SEQ ID NO: 379) | 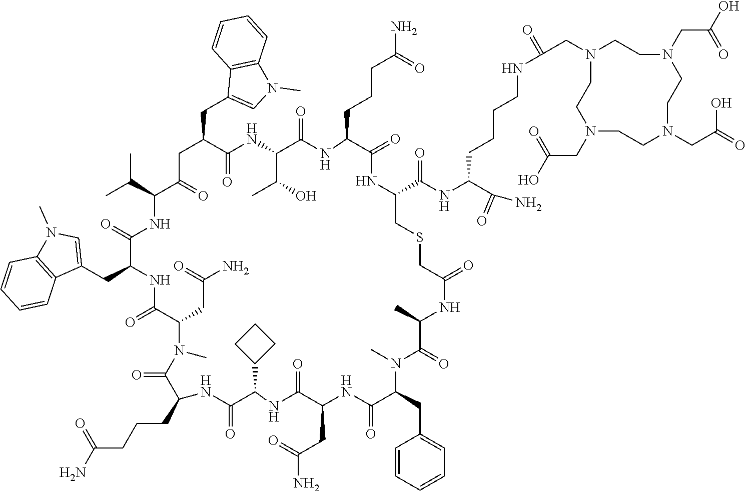 | HPLC-MS (Method A- B): 4.17 min ESI-MS m/z: 740.1 [M + 3H]3+ |
| PDC_EphA2- 00019437- C203 (SEQ ID NO: 389) | 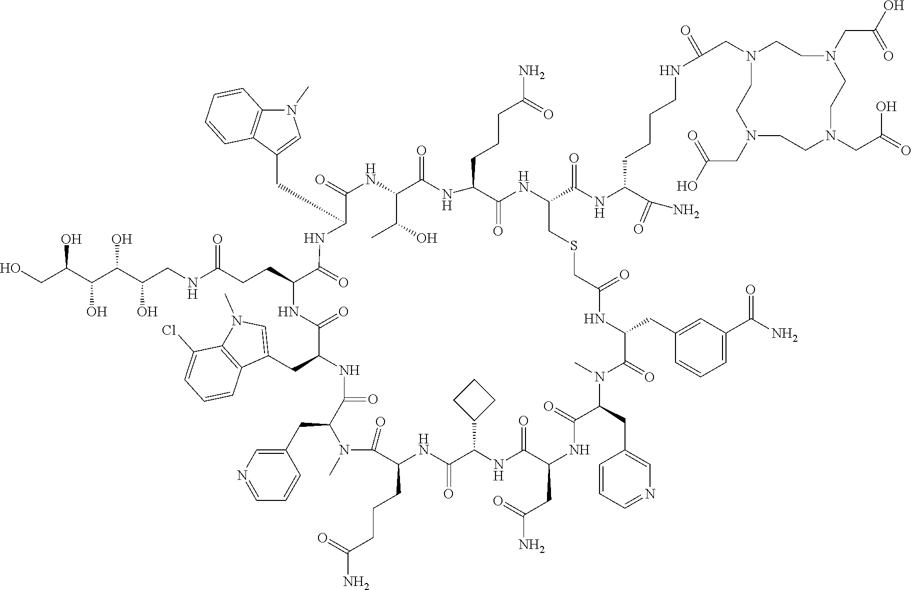 | HPLC-MS (Method A- A): 5.38 min ESI-MS m/z: 843.4 [M + 3H]3+ |
| PDC_EphA2- 00022593- C203 (SEQ ID NO: 390) | 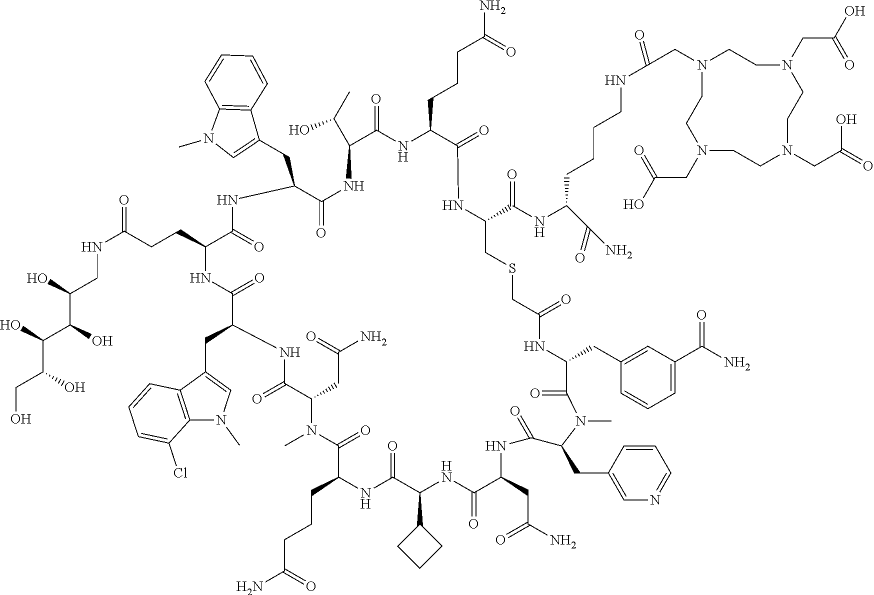 | HPLC-MS (Method A- A): 5.62 min ESI-MS m/z: 832.0 [M + 3H]3+ |
| PDC_EphA2- 00022594- C203 (SEQ ID NO: 391) | 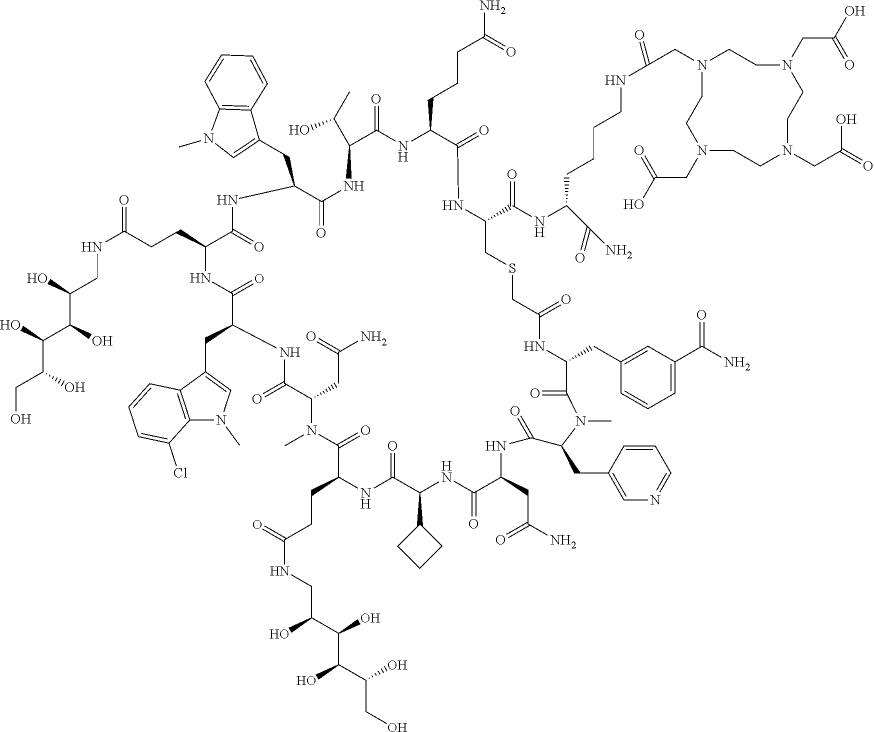 | HPLC-MS (Method A- A): 5.48 min ESI-MS m/z: 882.0 [M + 3H]3+ |
| PDC_EphA2- 00022595- C203 (SEQ ID NO: 392) | 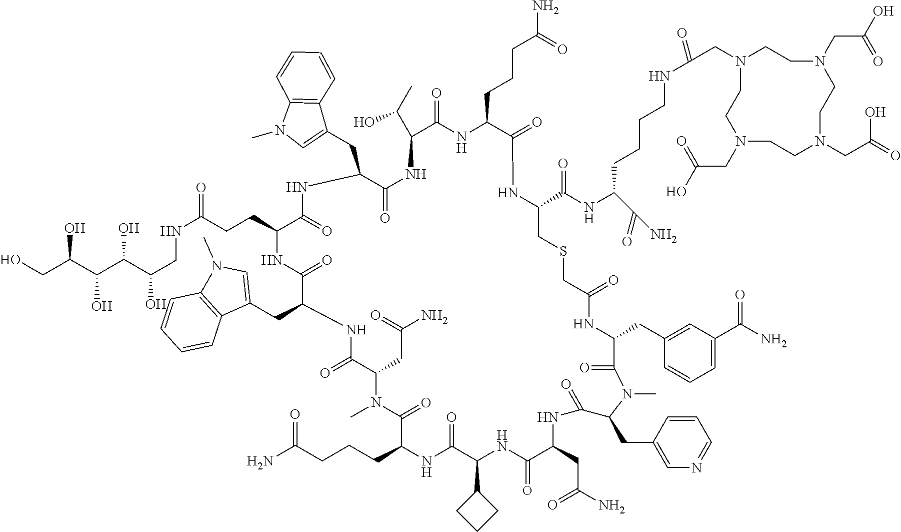 | HPLC-MS (Method A- A): 5.14 min ESI-MS m/z: 820.6 [M + 3H]3+ |
| PDC_EphA2- 00019440- C203 (SEQ ID NO: 393) | 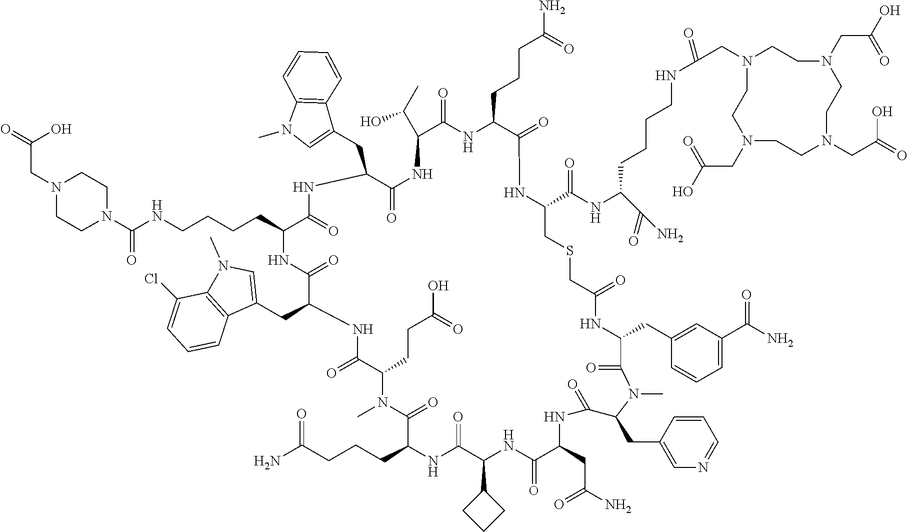 | HPLC-MS (Method A- A): 5.72 min ESI-MS m/z: 839.1 [M + 3H]3+ |
| PDC_EphA2- 00022612- C203 (SEQ ID NO: 394) | 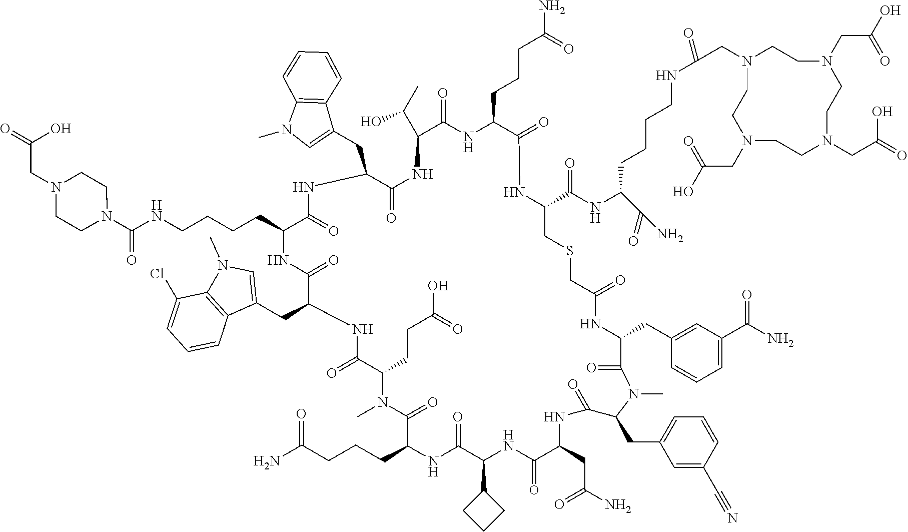 | HPLC-MS (Method A- B): 4.15 min ESI-MS m/z: 847.3 [M + 3H]3+ |
| PDC_EphA2- 00022613- C203 (SEQ ID NO: 395) | 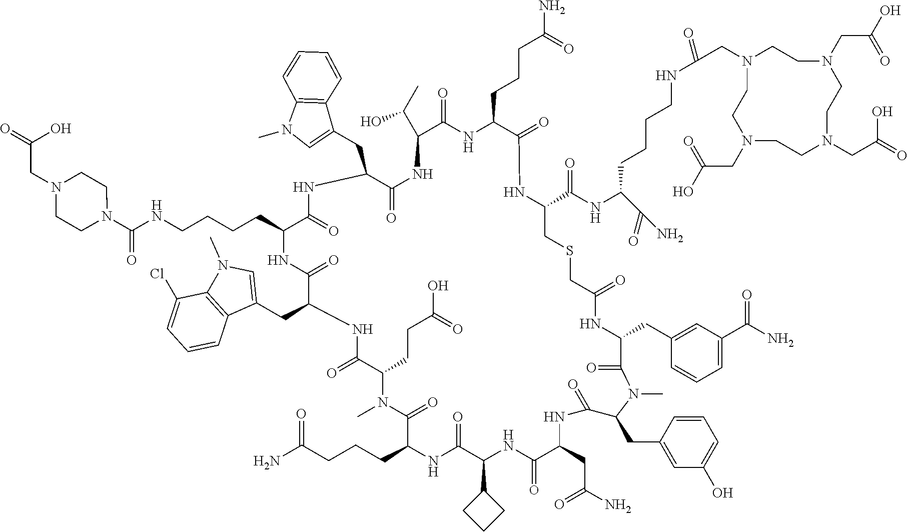 | HPLC-MS (Method A- B): 3.78 min ESI-MS m/z: 844.3 [M + 3H]3+ |
| PDC_EphA2- 00022614- C203 (SEQ ID NO: 396) | 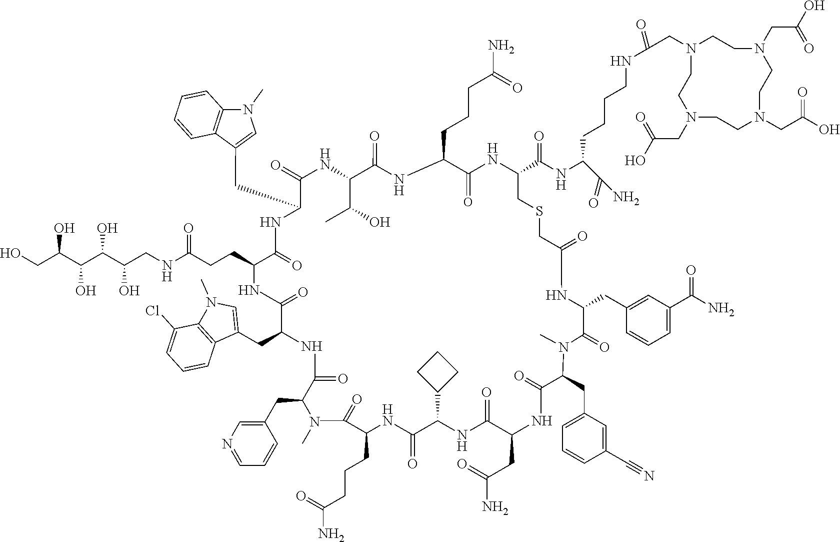 | HPLC-MS (Method A- B): 4.12 min ESI-MS m/z: 1276.4 [M + 2H]2+ |
| PDC_EphA2- 00022615- C203 (SEQ ID NO: 397) | 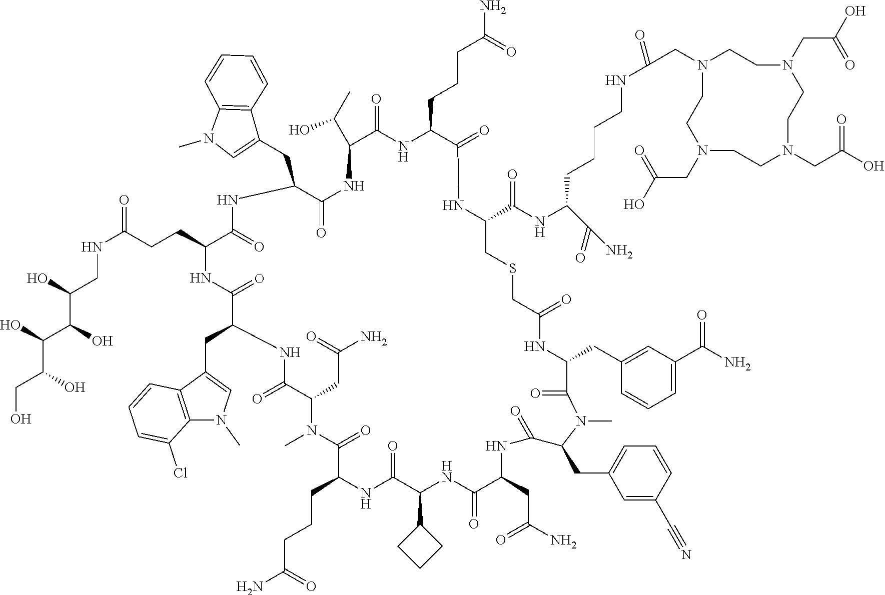 | HPLC-MS (Method A- B): 4.38 min ESI-MS m/z: 1259.4 [M + 2H]2+ |
| PDC_EphA2- 00013703- C203 (SEQ ID NO: 386) | 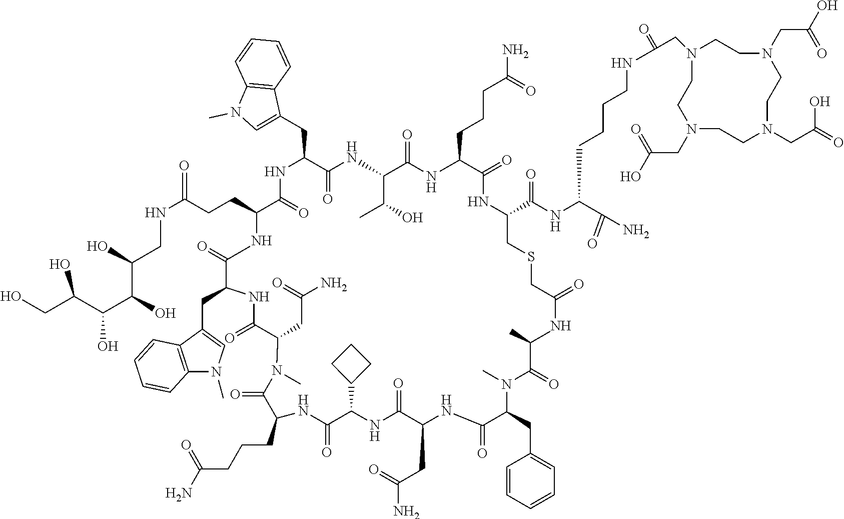 | HPLC-MS (Method A- B): 3.96 min ESI-MS m/z: 1170.2 [M + 2H]2+ |
| PDC_EphA2- 00013704- C203 (SEQ ID NO: 387) | 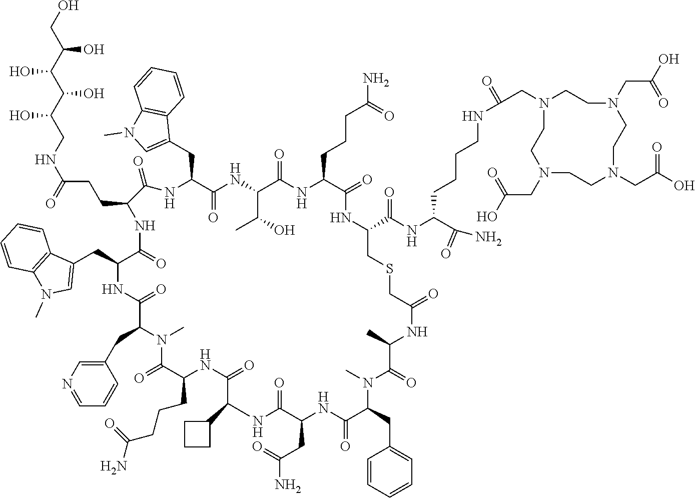 | HPLC-MS (Method A- B): 3.57 min ESI-MS m/z: 791.8 [M + 3H]3+ |
| PDC_EphA2- 00013705- C203 (SEQ ID NO: 388) | 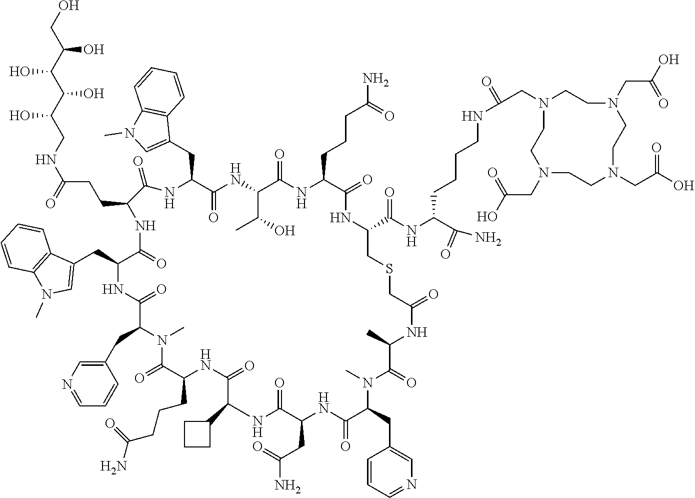 [SMILES: C[C@H]1NC(=O)CSC[C@@H](C(=O)N[C@H](CCCCNC(=O)CN2CCN(CC(=O)O)CCN(CC(=O)O)CCN(CC(=O)O)CC2)C(N)=O)NC(=O)[C@H](CCCC(N)=O)NC(=O)[C@H]([C@@H](C)O)NC(=O)[C@H](Cc2cn(C)c3ccccc23)NC(=O)[C@H](CCC(=O)NC[C@H](O)[C@@H](O)[C@H](O)[C@H](O)CO)NC(=O)[C@H](Cc2cn(C)c3ccccc23)NC(=O)[C@H](Cc2cccnc2)N(C)C(=O)[C@H](CCCC(N)=O)NC(=O)[C@H](C2CCC2)NC(=O)[C@H](CC(N)=O)NC(=O)[C@H](Cc2cccnc2)N(C)C1=O] | HPLC-MS (Method A- A): 4.64 min ESI-MS m/z: 1187.6 [M + 2H]2+ |
| PDC_EphA2- 00026424- C203 (SEQ ID NO: 398) | 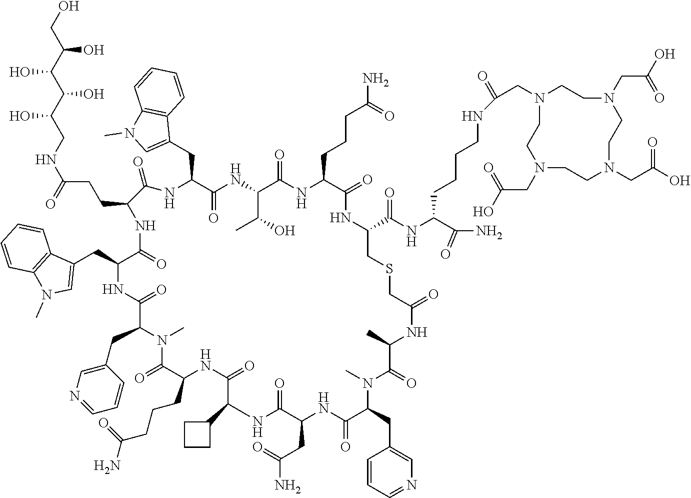 | HPLC-MS (Method A- A): 5.54 min ESI-MS m/z: 825.2 [M + 3H]3+ |
| PDC_EphA2- 00026425- C203 (SEQ ID NO: 399) | 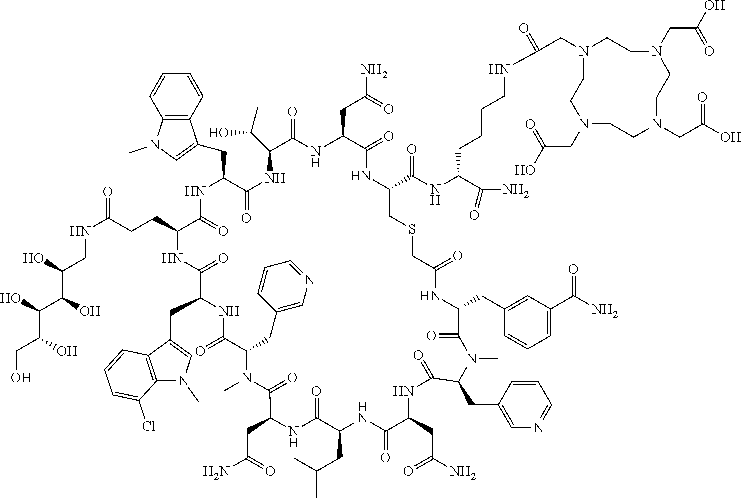 | HPLC-MS (Method A- A): 5.79 min ESI-MS m/z: 1220.4 [M + 2H]2+ |
| PDC_EphA2- 00026426- C203 (SEQ ID NO: 400) | 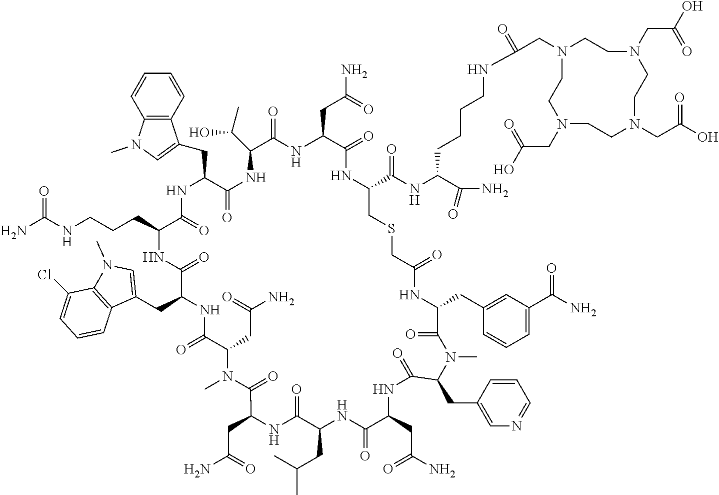 | HPLC-MS (Method A- B): 3.27 min ESI-MS m/z: 768.9 [M + 3H]3+ |
| PDC_EphA2- 00010011- C202 (SEQ ID NO: 377) | 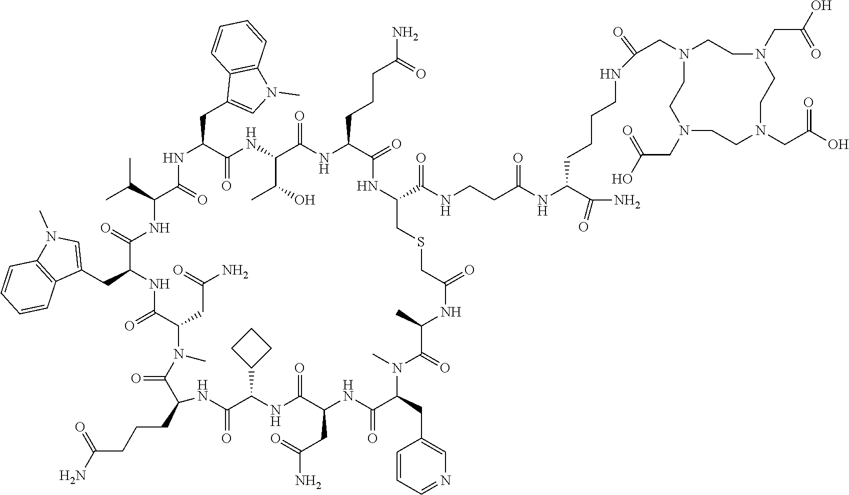 | HPLC-MS (Method A- B): 2.64 min ESI-MS m/z: 740.5 [M + 3H]3+ |
| PDC_EphA2- 00010012- C202 (SEQ ID NO: 378) | 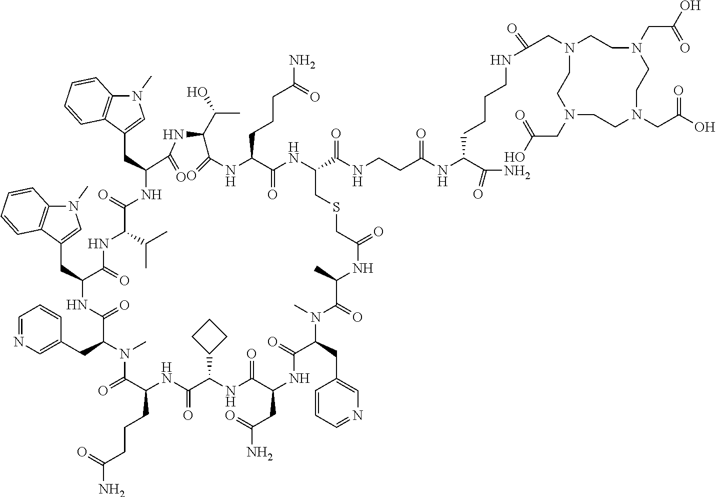 | HPLC-MS (Method A- A): 5.31 min ESI-MS m/z: 751.5 [M + 3H]3+ |
| PDC_EphA2- 00013693- C202 (SEQ ID NO: 379) | 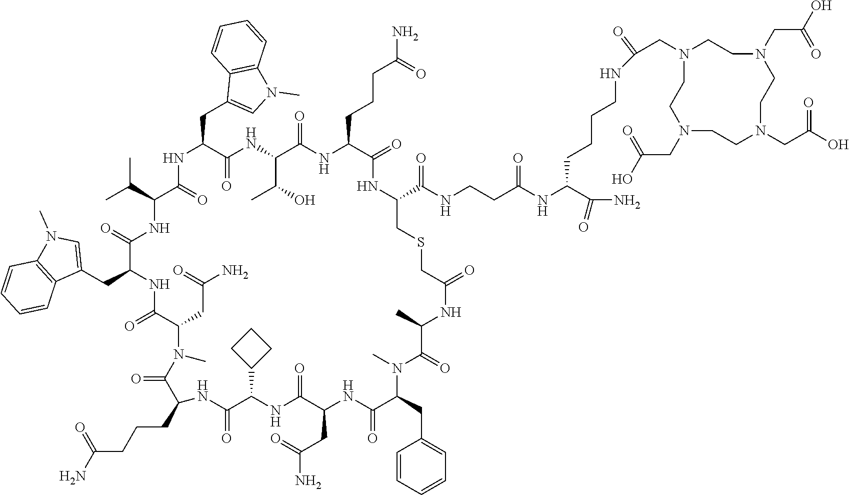 | HPLC-MS (Method A- B): 4.17 min ESI-MS m/z: 740.1 [M + 3H]3+ |
| PDC_EphA2- 00026628 (SEQ ID NO: 401) | 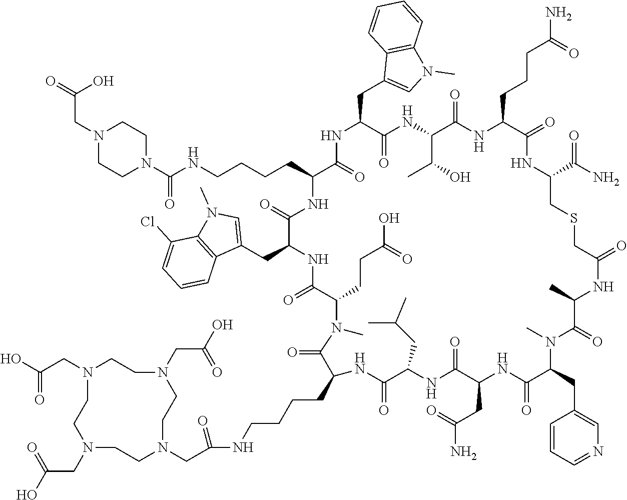 | HPLC-MS (Method A- B): 3.14 min ESI-MS m/z: 752.6 [M + 3H]3+ |
| PDC_EphA2- 00026629 (SEQ ID NO: 402) | 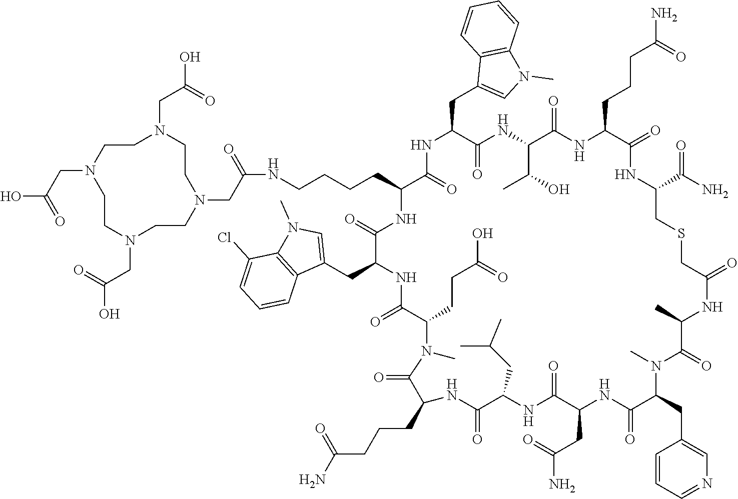 | HPLC-MS (Method A- B): 3.31 min ESI-MS m/z: 700.5 [M + 3H]3+ |
| PDC_EphA2- 00026630 (SEQ ID NO: 403) | 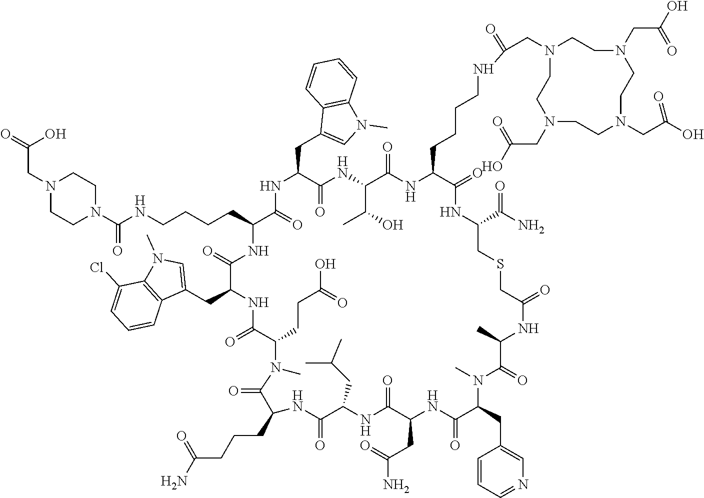 | HPLC-MS (Method A- B): 3.16 min ESI-MS m/z: 752.5 [M + 3H]3+ |
| PDC_EphA2- 00026708 (SEQ ID NO: 404) | 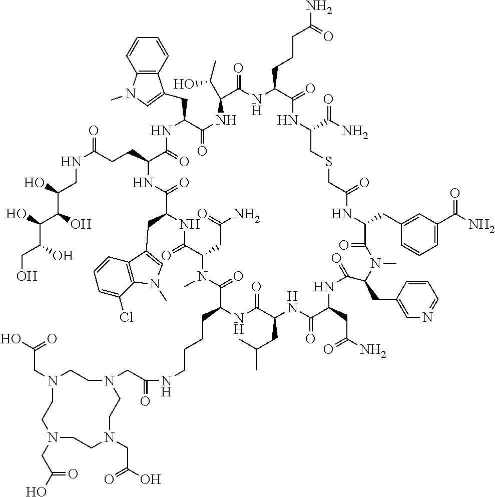 | HPLC-MS (Method A- B): 3.14 min ESI-MS m/z: 785.3 [M + 3H]3+ |
| PDC_EphA2- 00026709 (SEQ ID NO: 405) | 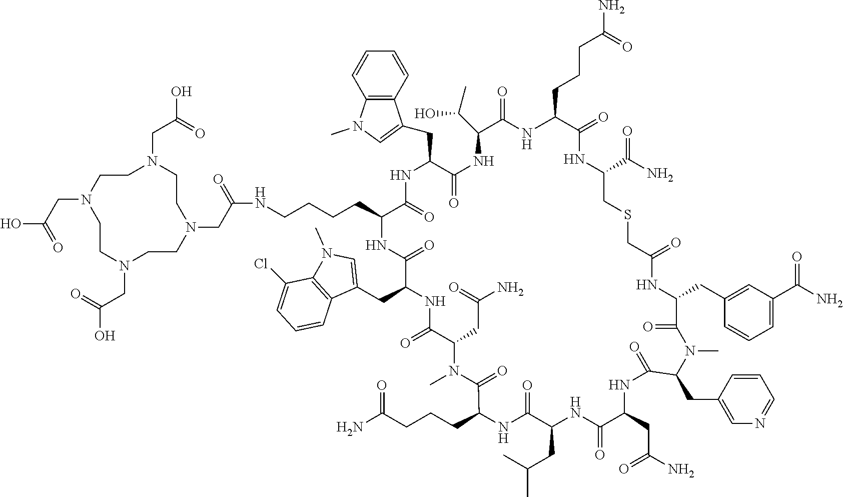 | HPLC-MS (Method A- A): 5.83 min ESI-MS m/z: 735.2 [M + 3H]3+ |
| PDC_EphA2- 00026710 (SEQ ID NO: 406) | 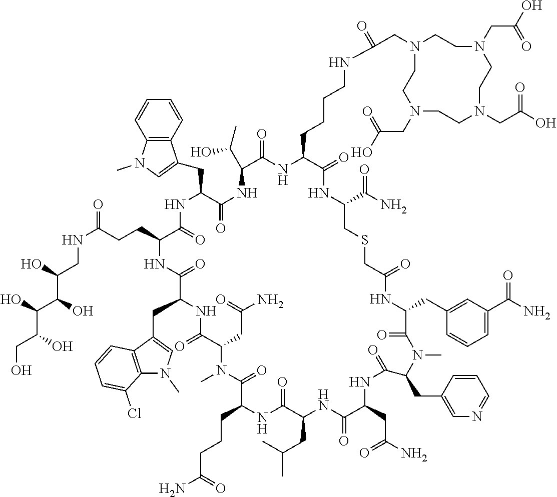 | HPLC-MS (Method A- A): 5.72 min ESI-MS m/z: 785.3 [M + 3H]3+ |
| PDC_EphA2- 00026711 (SEQ ID NO: 407) | 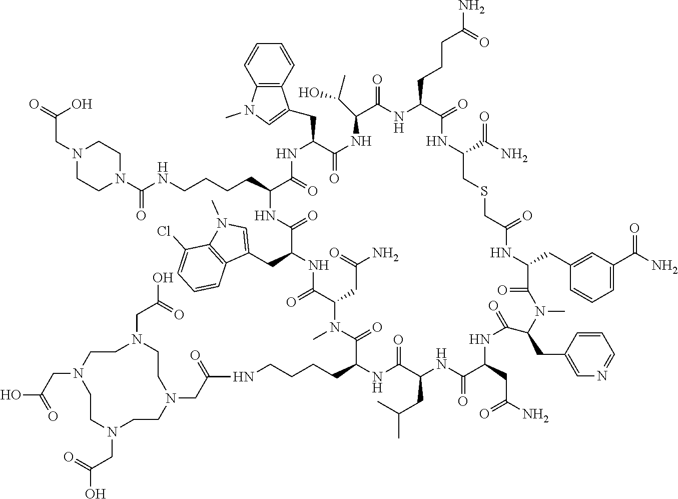 | HPLC-MS (Method A- B): 3.19 min ESI-MS m/z: 792.3 [M + 3H]3+ |
| PDC_EphA2- 00026712 (SEQ ID NO: 408) | 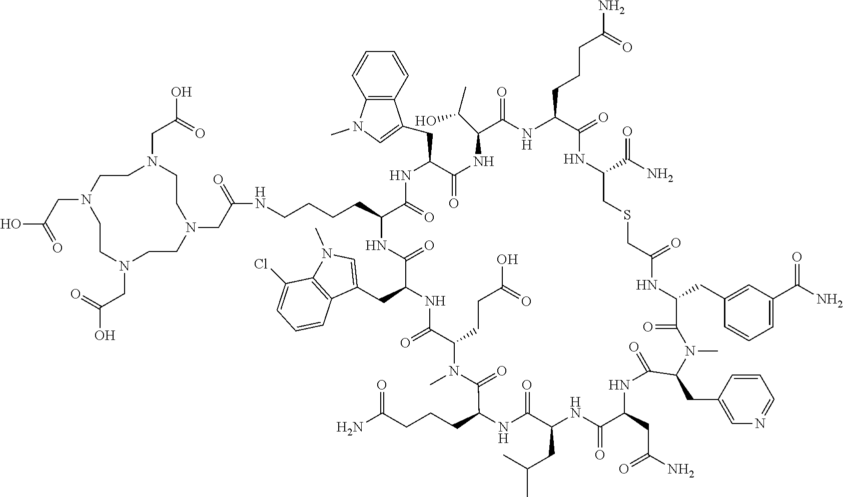 | HPLC-MS (Method A- B): 3.35 min ESI-MS m/z: 740.2 [M + 3H]3+ |
| PDC_EphA2- 00026713 (SEQ ID NO: 409) | 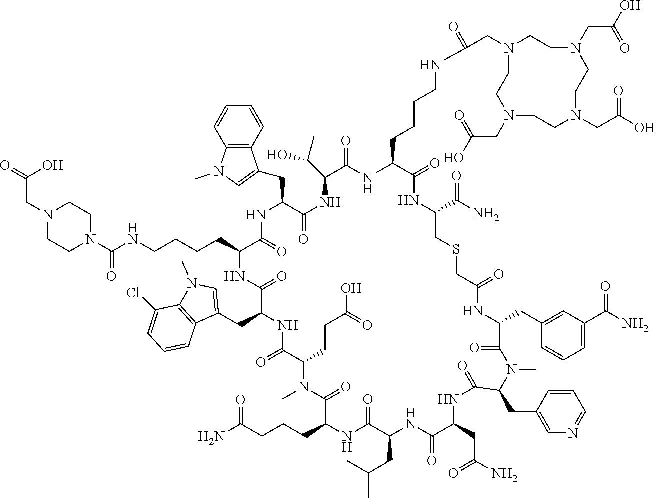 | HPLC-MS (Method A- A): 5.79 min ESI-MS m/z: 792.4 [M + 3H]3+ |
| PDC_EphA2- 00026768 (SEQ ID NO: 410) | 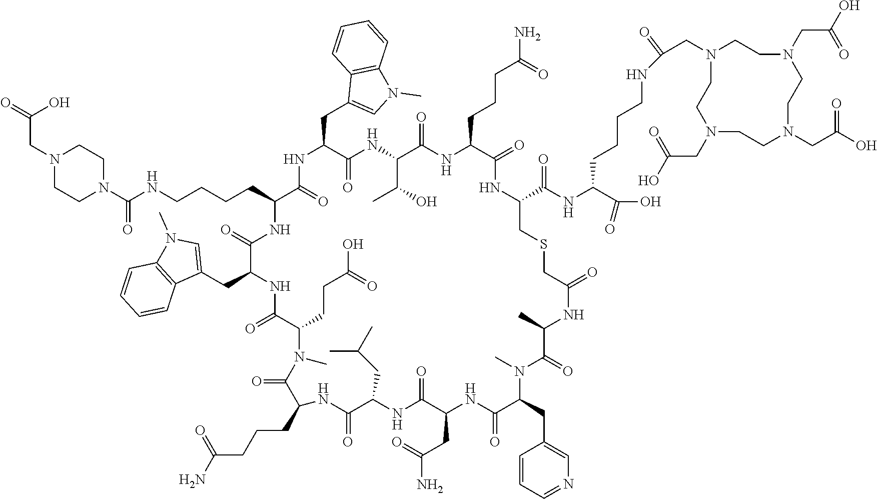 | HPLC-MS (Method A- A): 5.06 min ESI-MS m/z: 789.2 [M + 3H]3+ |
| PDC_EphA2- 00026769 (SEQ ID NO: 411) | 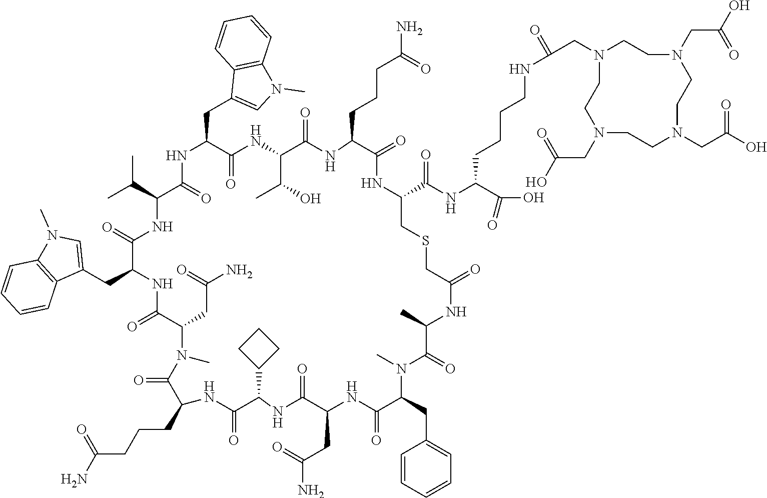 [SMILES: CC(C)[C@@H]1NC(=O)[C@H](Cc2cn(C)c3ccccc23)NC(=O)[C@H](CC(N)=O)N(C)C(=O)[C@H](CCCC(N)=O)NC(=O)[C@H](C2CCC2)NC(=O)[C@H](CC(N)=O)NC(=O)[C@H](Cc2ccccc2)N(C)C(=O)[C@@H](C)NC(=O)CSC[C@@H](C(=O)N[C@H](CCCCNC(=O)CN2CCN(CC(=O)O)CCN(CC(=O)O)CCN(CC(=O)O)CC2)C(=O)O)NC(=O)[C@H](CCCC(N)=O)NC(=O)[C@H]([C@@H](C)O)NC(=O)[C@H](Cc2cn(C)c3ccccc23)NC1=O] | HPLC-MS (Method A- B): 4.42 min ESI-MS m/z: 1074.0 [M + 2H]2+ |
| PDC_EphA2- 00026770 (SEQ ID NO: 412) | 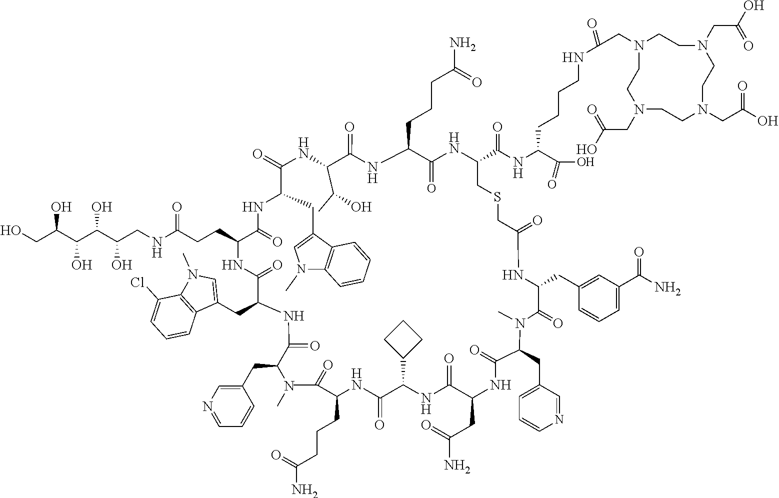 | HPLC-MS (Method A- A): 5.12 min ESI-MS m/z: 844.0 [M + 3H]3+ |
| PDC_EphA2- 00026771 (SEQ ID NO: 413) | 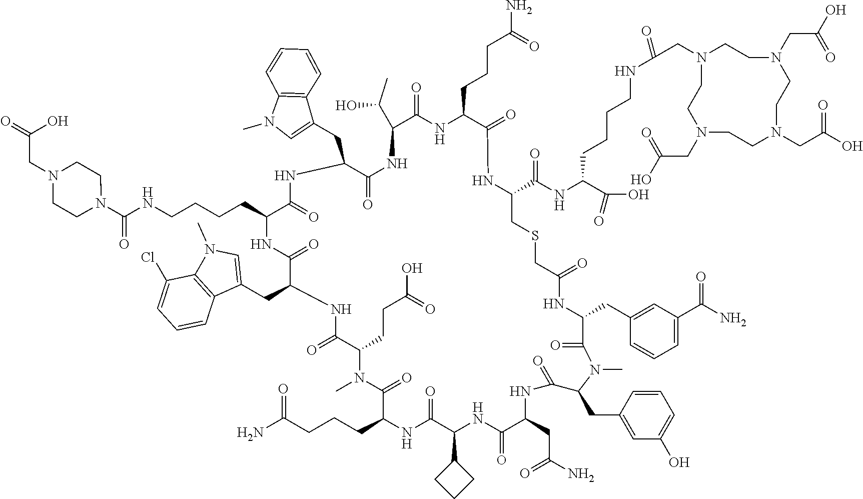 | HPLC-MS (Method A- B): 4.03 min ESI-MS m/z: 1266.0 [M + 2H]2+ |
| PDC_EphA2- 00001417- C204 (SEQ ID NO: 424) | 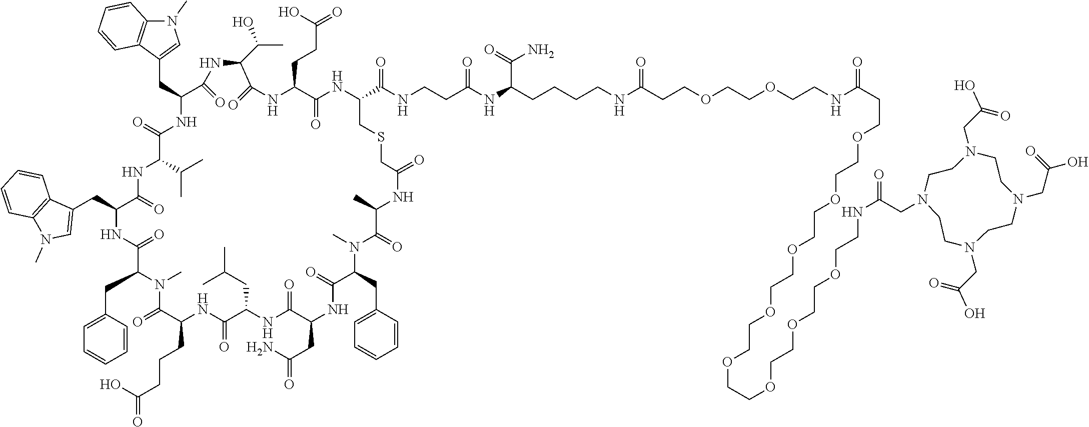 | HPLC-MS (Method A- B): 5.40 min ESI-MS m/z: 941.8 [M + 3H]3+ |
| PDC_EphA2- 00007196- C204 (SEQ ID NO: 370) | 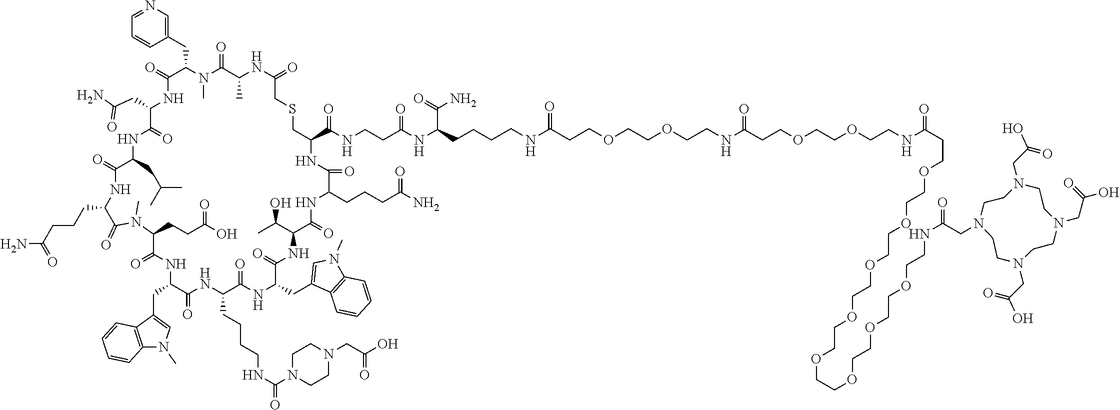 | HPLC-MS (Method A- A): 5.45 min ESI-MS m/z: 1006.3 [M + 3H]3+ |
| PDC_EphA2- 00008093- C204 (SEQ ID NO: 371) | 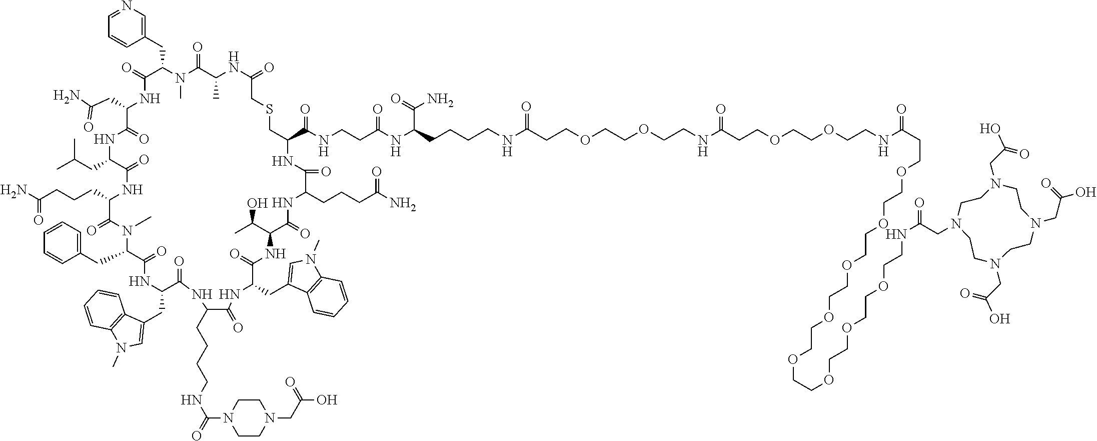 | HPLC-MS (Method A- B): 3.33 min ESI-MS m/z: 1012.4 [M + 3H]3+ |
| PDC_EphA2- 00008094- C204 (SEQ ID NO: 372) | 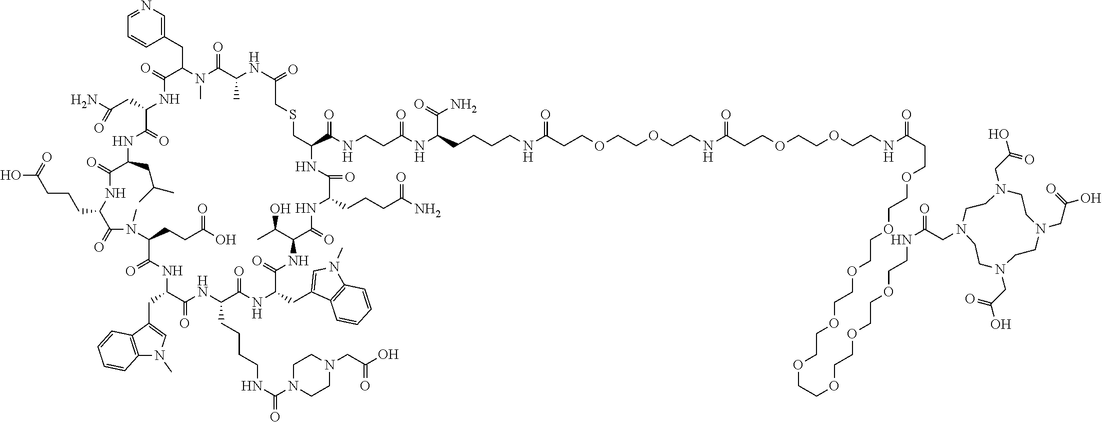 | HPLC-MS (Method A- A): 5.34 min ESI-MS m/z: 1007.0 [M + 3H]3+ |
| PDC_EphA2- 00010011- C204 (SEQ ID NO: 380) | 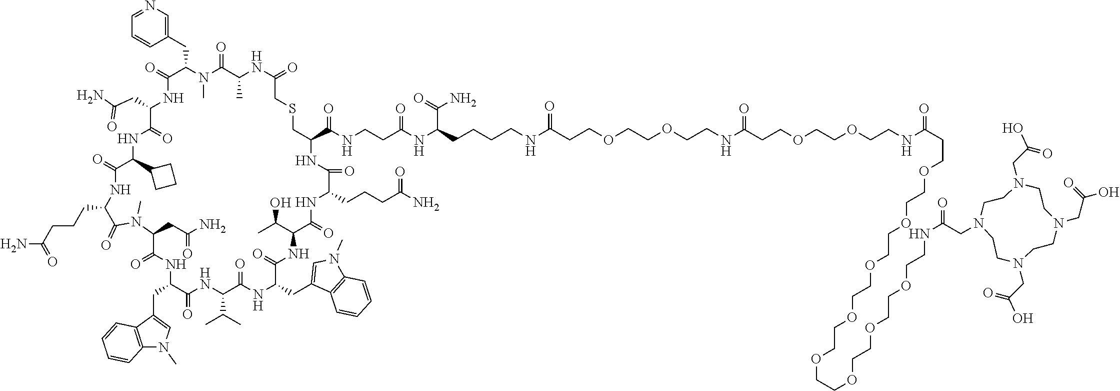 | HPLC-MS (Method A- B): 2.88 min ESI-MS m/z: 934.7 [M + 3H]3+ |
| PDC_EphA2- 00010012- C204 (SEQ ID NO: 381) | 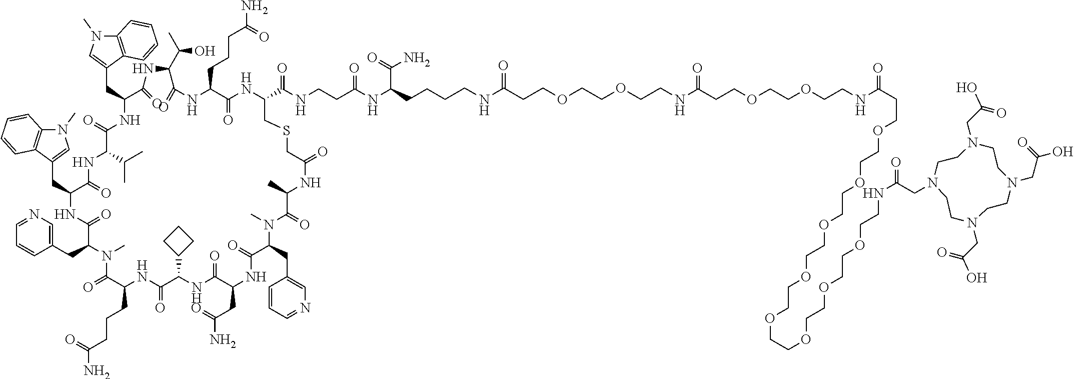 | HPLC-MS (Method A- A): 5.52 min ESI-MS m/z: 945.7 [M + 3H]3+ |
| PDC_EphA2- 00013693- C204 (SEQ ID NO: 382) | 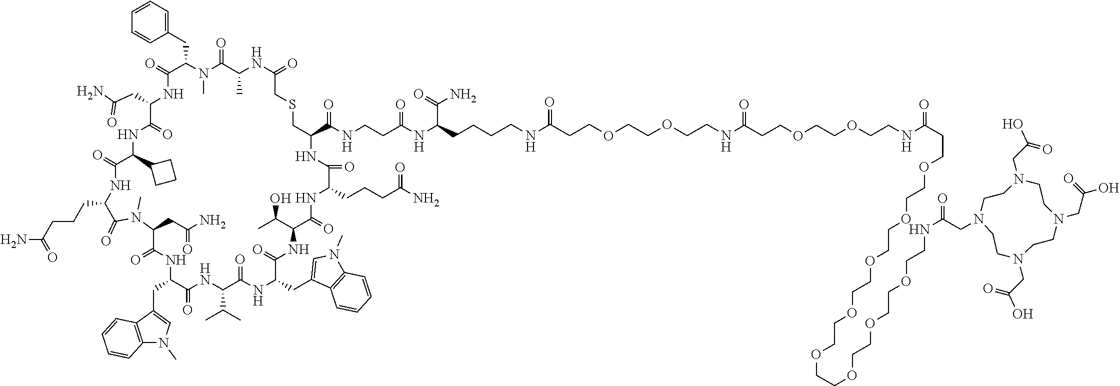 | HPLC-MS (Method A- B): 4.24 min ESI-MS m/z: 934.3 [M + 3H]3+ |
| PDC_EphA2- 00008093- C210 (SEQ ID NO: 374) | 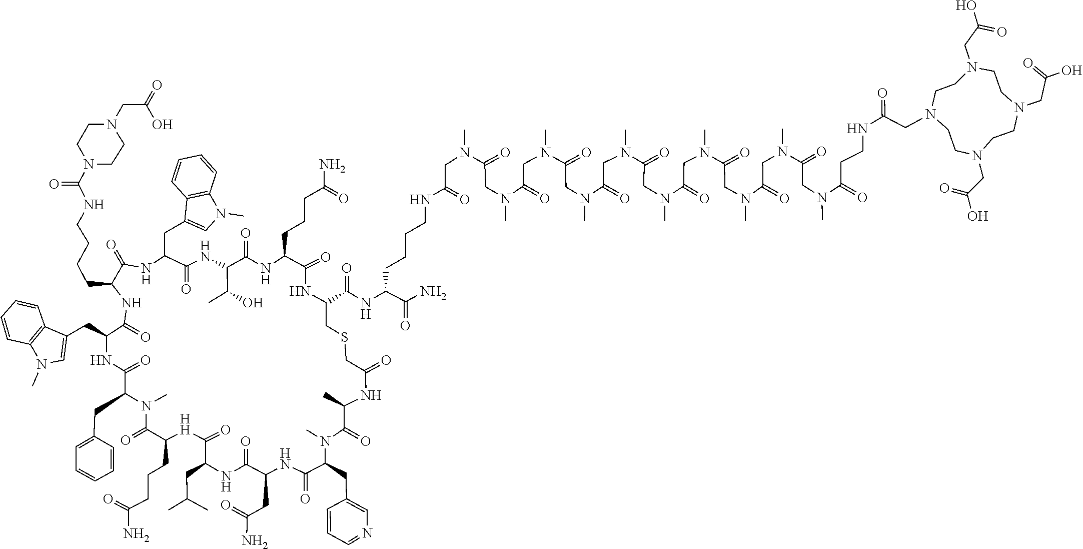 | HPLC-MS (Method A- B): 3.11 min ESI-MS m/z: 1055.0 [M + 3H]3+ |
| PDC_EphA2- 00007196- C210 (SEQ ID NO: 376) | 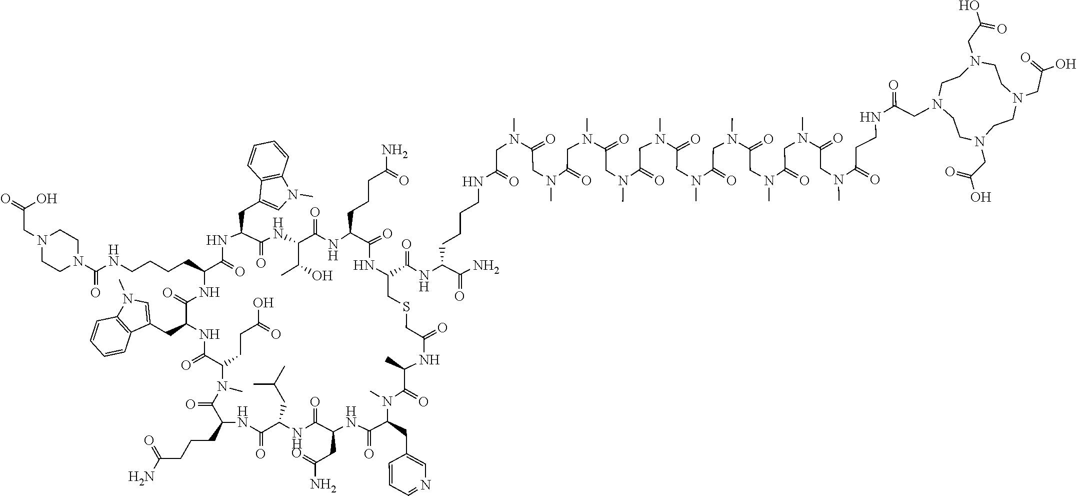 | HPLC-MS (Method A- A): 5.22 min ESI-MS m/z: 1049.1 [M + 3H]3+ |

### Example B19: PDC_EphA2-00007196-C302 (SEQ ID NOS 200 and 292)

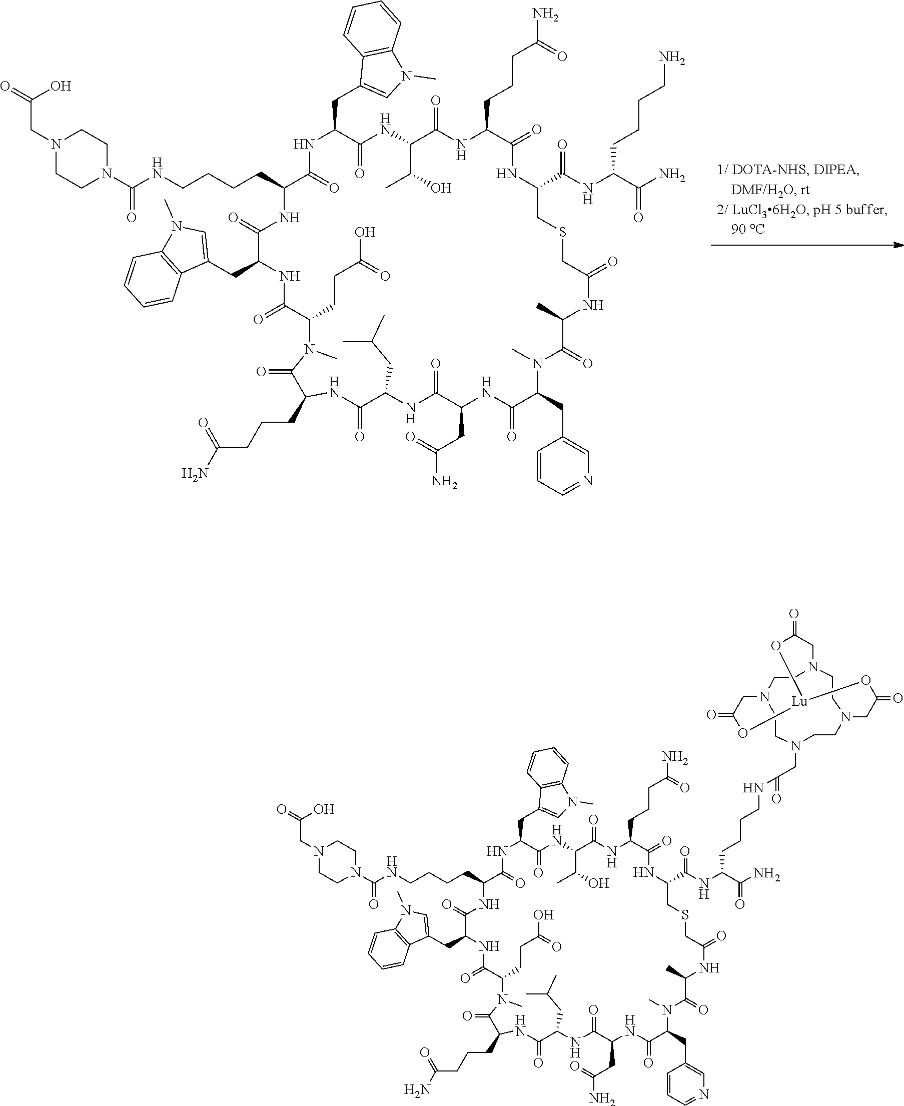

To a solution of Example-B2 (PDC_EphA2-00007196-C002) (60.0 mg, 0.026 mmol) in DMF (1294 μL) at 0 °C were added DIPEA (40.7 μL, 0.233 mmol) and 0.12 M aq. DOTA-NHS ester (1294 μL, 0.155 mmol). The reaction mixture was stirred at rt for 0.5 h. Then to the mixture was added DIPEA (40.7 μL, 0.233 mmol) and stirred at rt for 0.5 h. To the mixture was added a solution of LuCl3 6H2O (5.69 mL, 0.285 mmol) in 50 mM NH4OAc buffer (pH5) at rt. The mixture was stirred at 90 °C for 1 h. The resulting mixture was purified by preparative reverse phase C18-HPLC (Method P-A). Pure fractions were combined and lyophilized to afford the title compound. HPLC-MS (Method A-A): 5.79 min, ESI-MS m/z: 845.8 [M+3H]3+, UV-area (TIC): 98%.

### Example B20

The following Examples were prepared by following similar methods described in the examples above. The amino acid sequence for each peptide was shown in Table B6 and Table B6-1. The term “Term” means the functional group at C-terminus of the peptide.

TABLE B14

| Example No. | Structure | Analytics |
|---|---|---|
| PDC_EphA2- 00008093- C302 (SEQ ID NO: 288) | 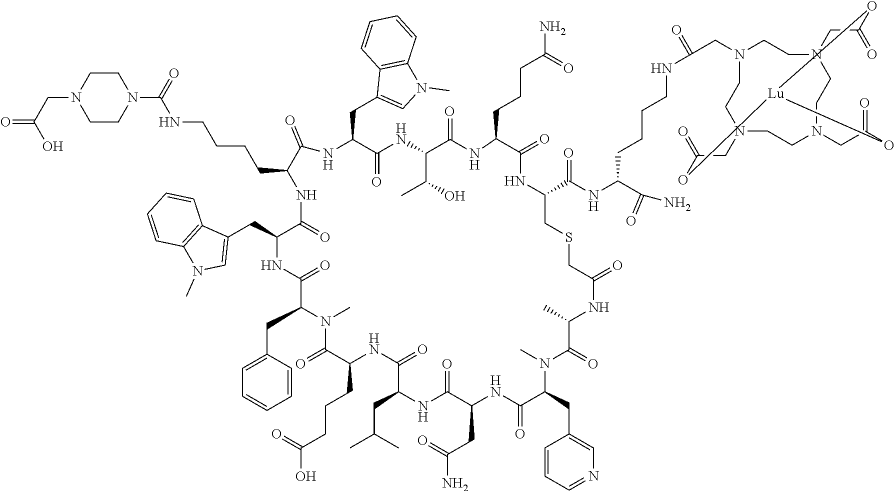 | HPLC-MS (Method A-B): 3.42 min ESI-MS m/z: 851.7 [M + 3H]3+ |
| PDC_EphA2- 00010011- C302 (SEQ ID NO: 243) | 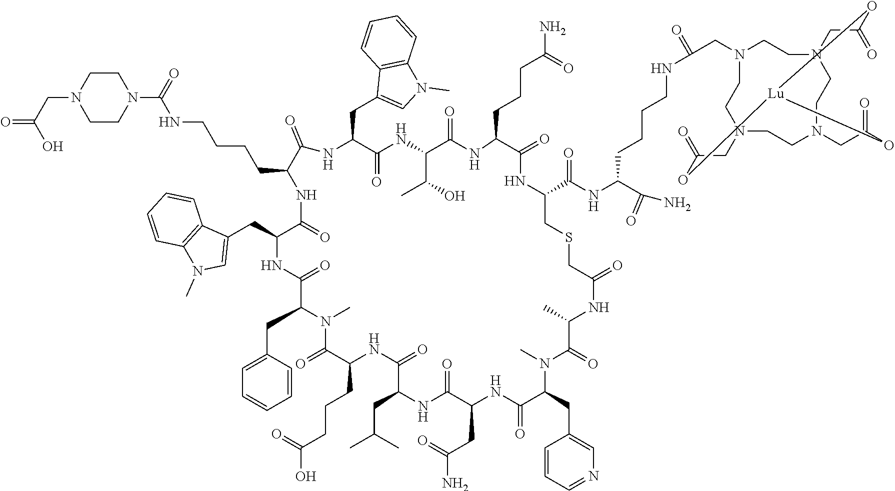 | HPLC-MS (Method A-B): 3.17 min ESI-MS m/z: 773.7 [M + 3H]3+ |
| PDC_EphA2- 00010012- C302 (SEQ ID NO: 245) | 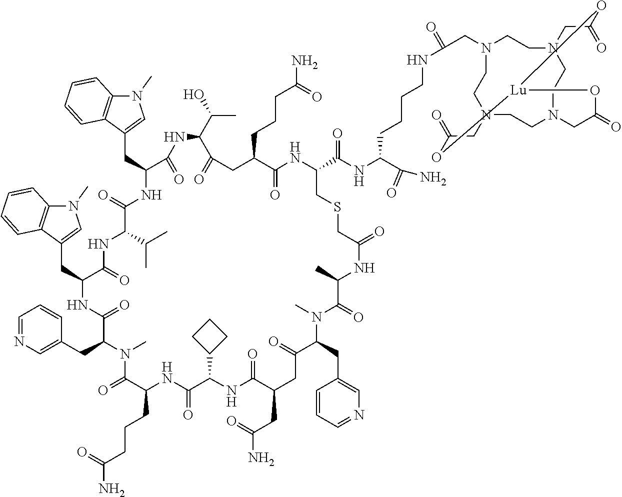 | HPLC-MS (Method A-A): 5.28 min ESI-MS m/z: 785.4 [M + 3H]3+ |
| PDC_EphA2- 00013693- C302 (SEQ ID NO: 317) | 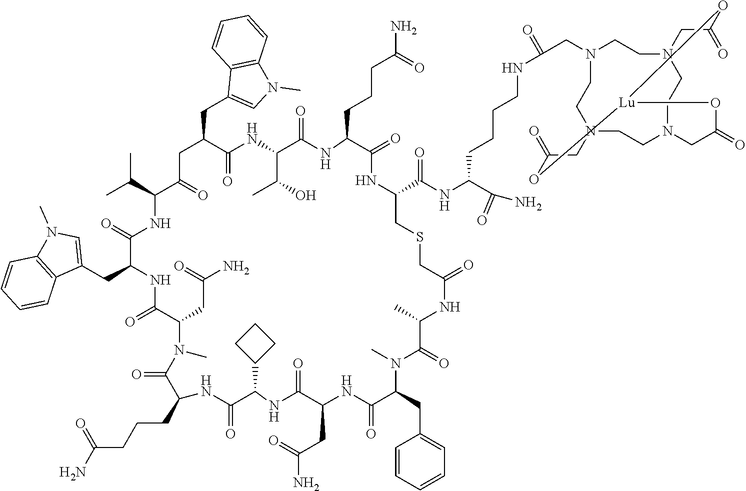 | HPLC-MS (Method A-B): 4.69 min ESI-MS m/z: 773.4 [M + 3H]3+ |
| PDC_EphA2- 00019437- C302 (SEQ ID NO: 323) | 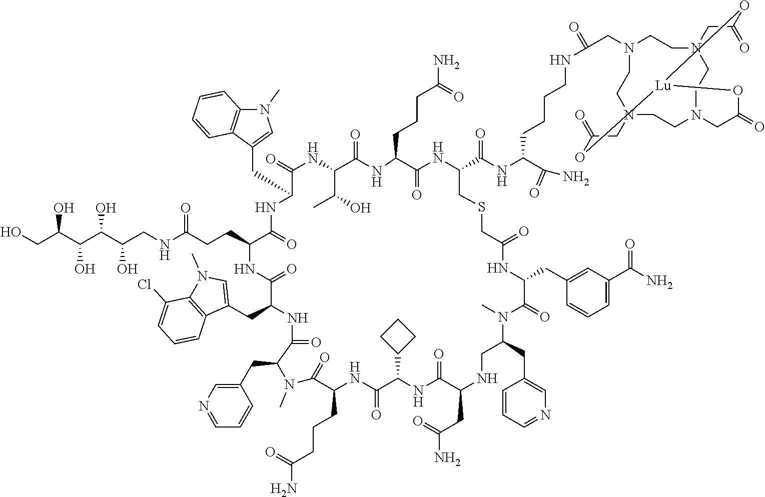 | HPLC-MS (Method A-A): 5.52 min ESI-MS m/z: 900.7 [M + 3H]3+ |
| PDC_EphA2- 00022593- C302 (SEQ ID NO: 325) | 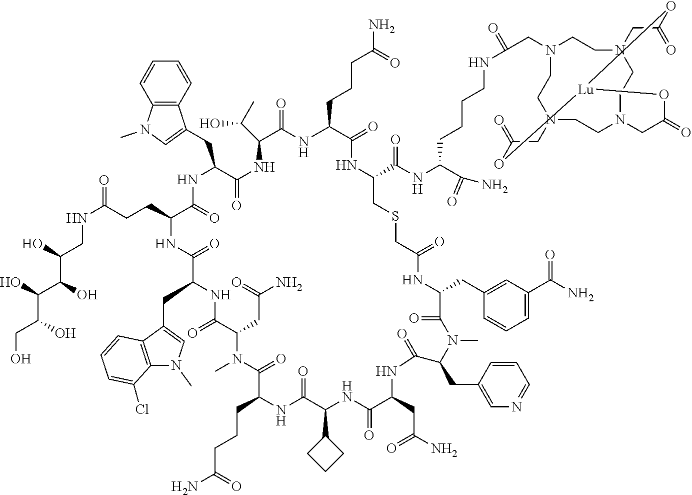 | HPLC-MS (Method A-B): 3.13 min ESI-MS m/z: 1333.3 [M + 2H]2+ |
| PDC_EphA2- 00022594- C302 (SEQ ID NO: 326) | 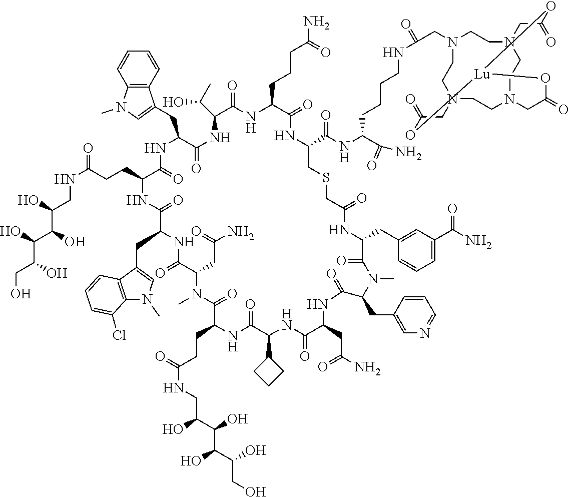 | HPLC-MS (Method A-A): 5.68 min ESI-MS m/z: 939.3 [M + 3H]3+ |
| PDC_EphA2- 00022595- C302 (SEQ ID NO: 347) | 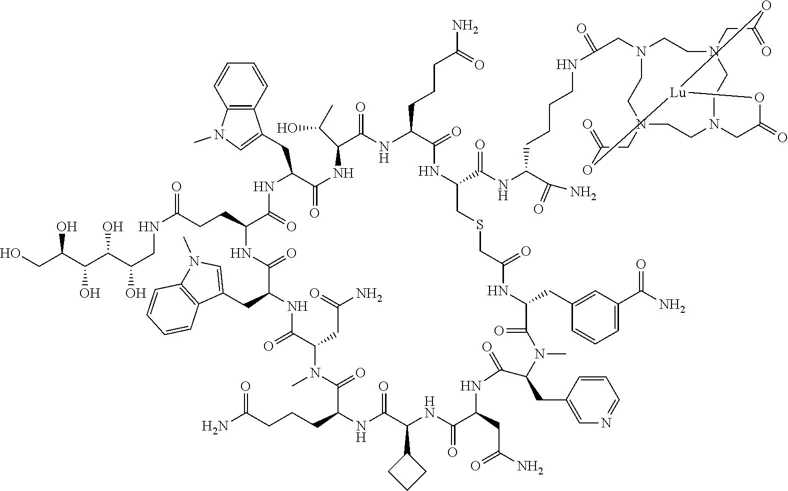 | HPLC-MS (Method A-A): 5.34 min ESI-MS m/z: 877.9 [M + 3H]3+ |
| PDC_EphA2- 00019440- C302 (SEQ ID NO: 329) | 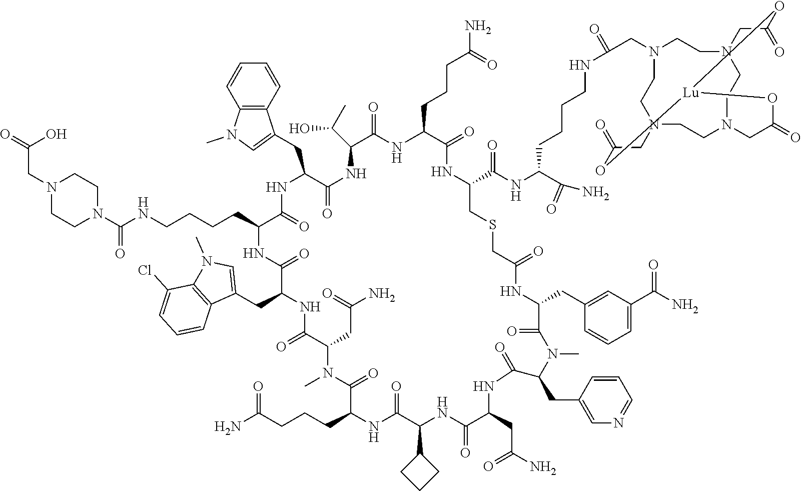 | HPLC-MS (Method A-A): 5.91 min ESI-MS m/z: 896.3 [M + 3H]3+ |
| PDC_EphA2- 00022612- C302 (SEQ ID NO: 348) | 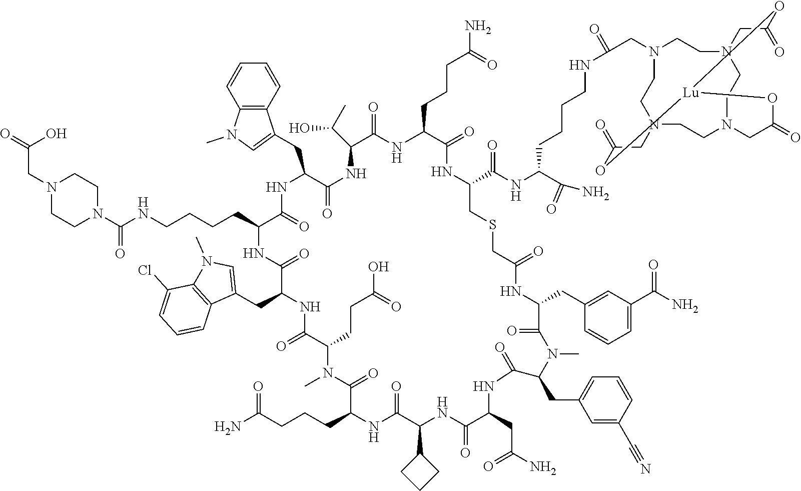 | HPLC-MS (Method A-B): 4.65 min ESI-MS m/z: 1355.9 [M + 2H]2+ |
| PDC_EphA2- 00022613- C302 (SEQ ID NO: 332) | 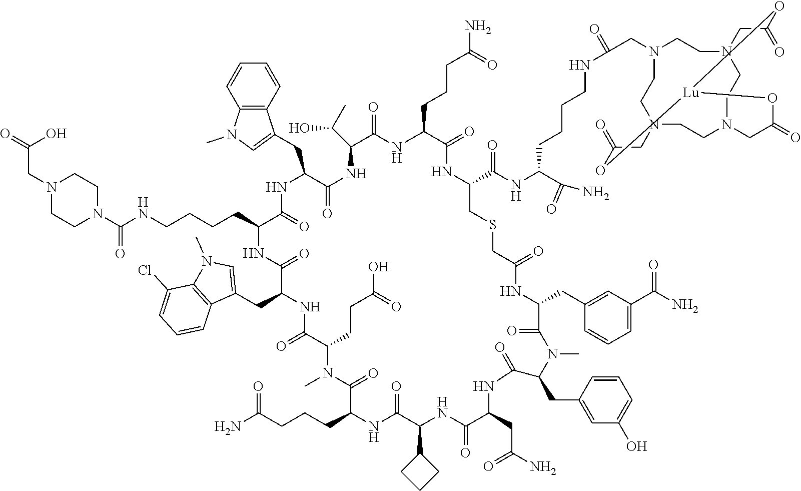 | HPLC-MS (Method A-B): 4.25 min ESI-MS m/z: 1351.4 [M + 2H]2+ |
| PDC_EphA2- 00022614- C302 (SEQ ID NO: 349) | 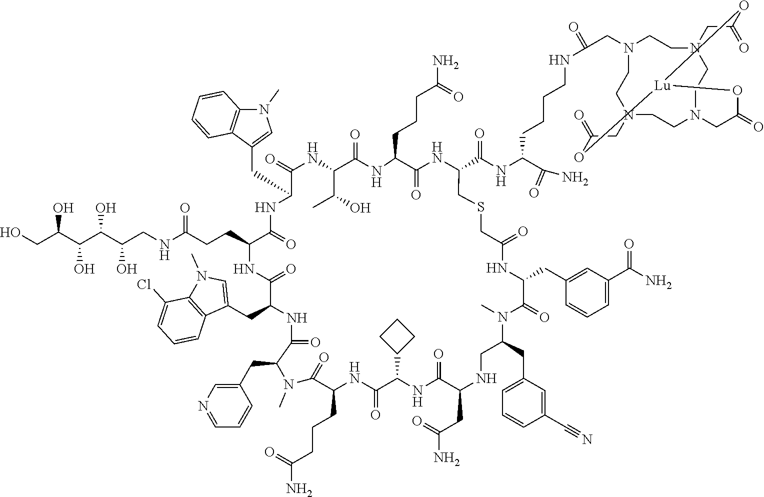 | HPLC-MS (Method A-B): 4.15 min ESI-MS m/z: 908.9 [M + 3H]3+ |
| PDC_EphA2- 00022615- C302 (SEQ ID NO: 350) | 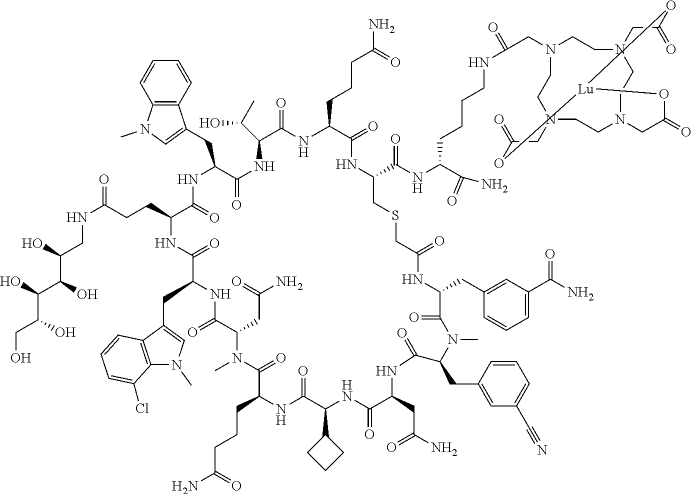 | HPLC-MS (Method A-B): 4.55 min ESI-MS m/z: 897.5 [M + 3H]3+ |
| PDC_EphA2- 00013703- C302 (SEQ ID NO: 318) | 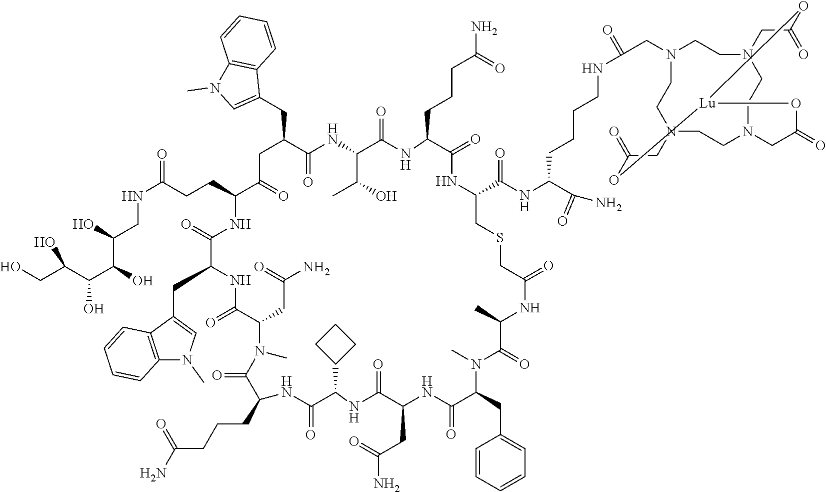 | HPLC-MS (Method A-B): 4.34 min ESI-MS m/z: 1256.2 [M + 2H]2+ |
| PDC_EphA2- 00013704- C302 (SEQ ID NO: 320) | 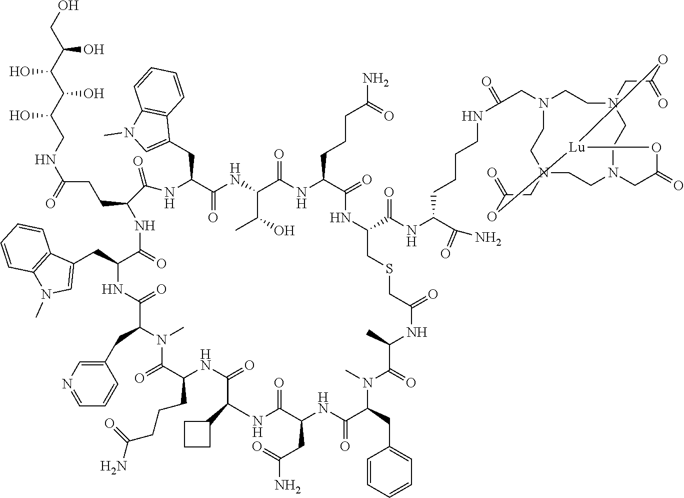 | HPLC-MS (Method A-B): 3.84 min ESI-MS m/z: 1273.2 [M + 2H]2+ |
| PDC_EphA2- 00013705- C302 (SEQ ID NO: 322) | 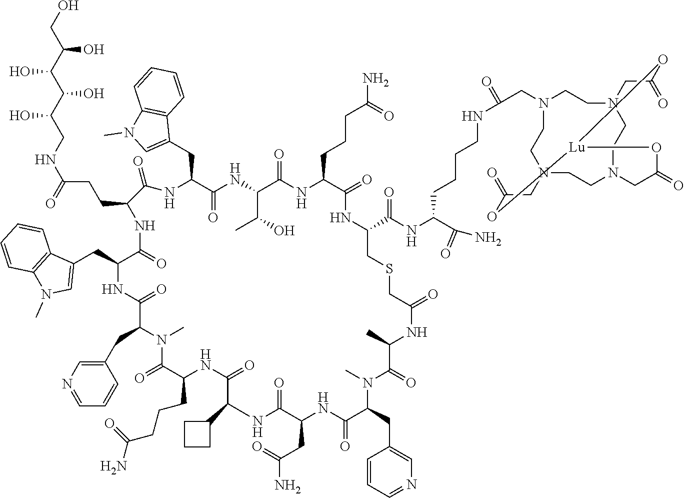 | HPLC-MS (Method A-A): 4.77 min ESI-MS m/z: 1273.6 [M + 2H]2+ |
| PDC_EphA2- 00026424- C302 (SEQ ID NO: 351) | 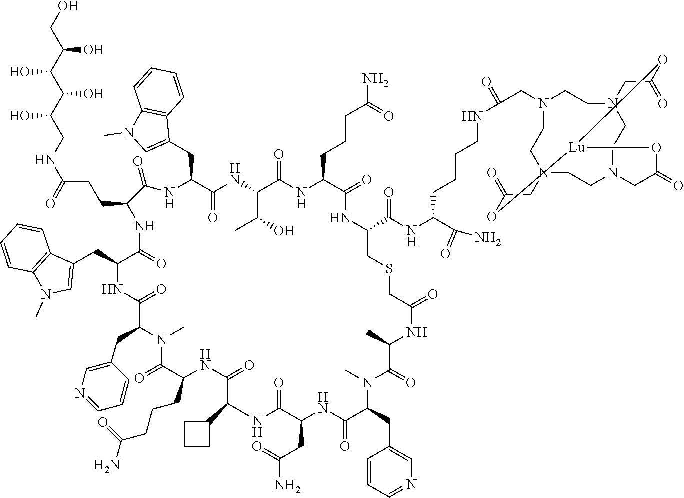 | HPLC-MS (Method A-A): 5.31 min ESI-MS m/z: 883.0 [M + 3H]3+ |
| PDC_EphA2- 00026425- C302 (SEQ ID NO: 352) | 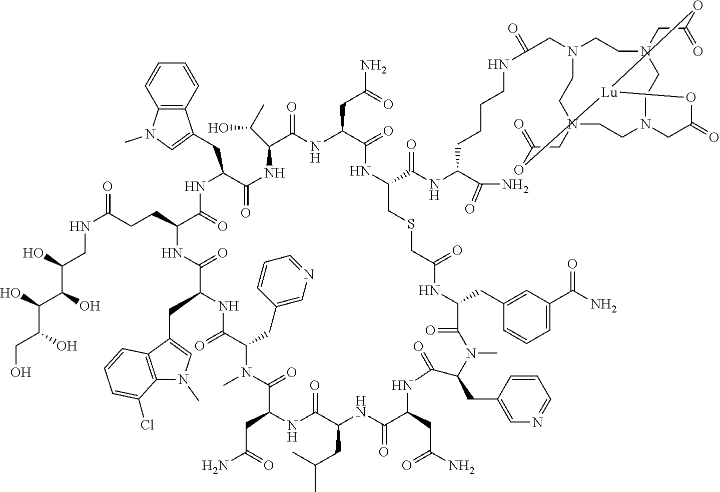 | HPLC-MS (Method A-B): 3.26 min ESI-MS m/z: 871.2 [M + 3H]3+ |
| PDC_EphA2- 00026426- C302 (SEQ ID NO: 353) | 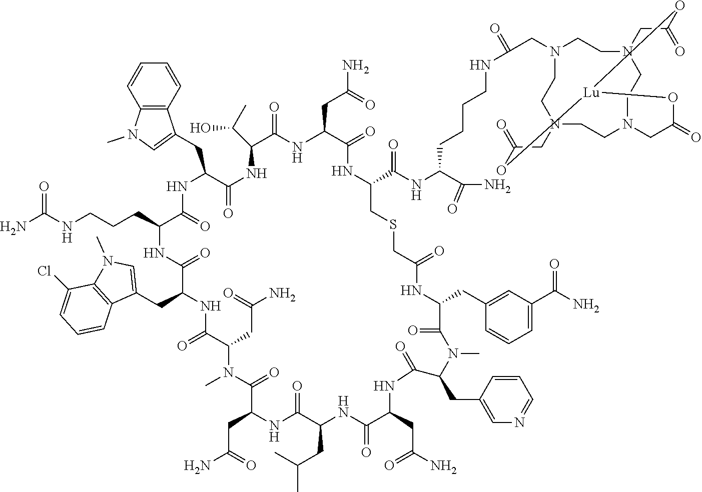 | HPLC-MS (Method A-B): 3.46 min ESI-MS m/z: 826.2 [M + 3H]3+ |
| PDC_EphA2- 00010011- C303 (SEQ ID NO: 354) | 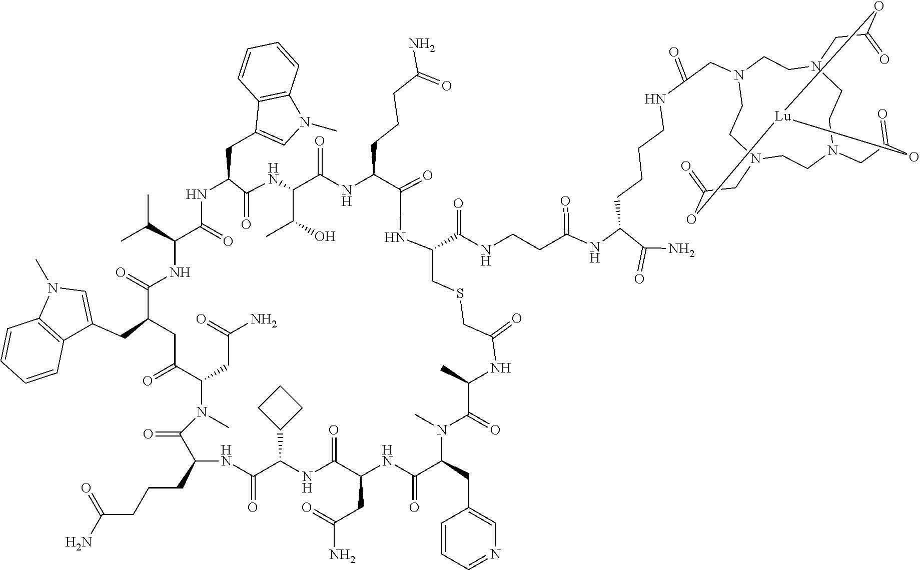 | HPLC-MS (Method A-B): 2.86 min ESI-MS m/z: 797.7 [M + 3H]3+ |
| PDC_EphA2- 00010012- C303 (SEQ ID NO: 355) | 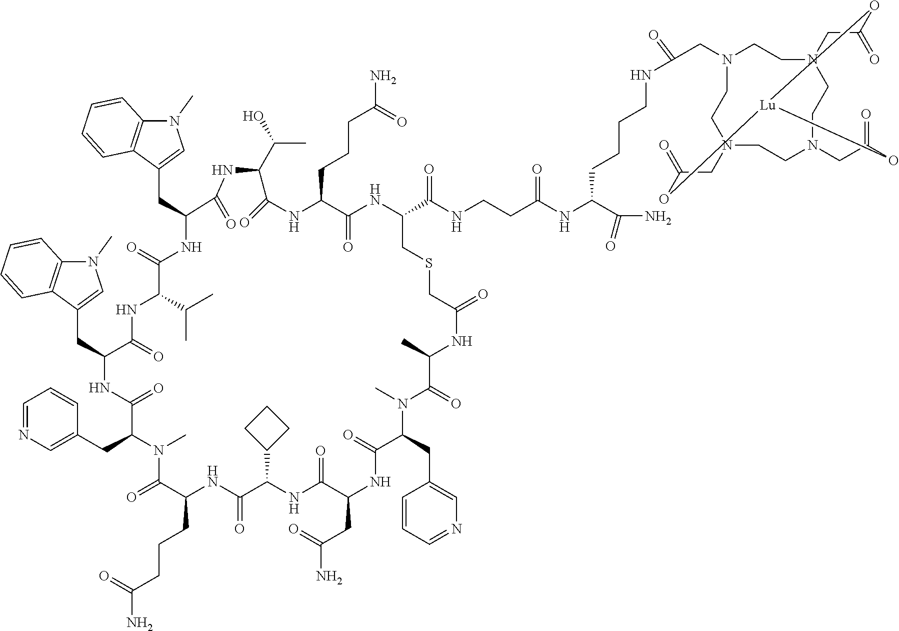 | HPLC-MS (Method A-A): 5.47 min ESI-MS m/z: 808.8 [M + 3H]3+ |
| PDC_EphA2- 00013693- C303 (SEQ ID NO: 315) | 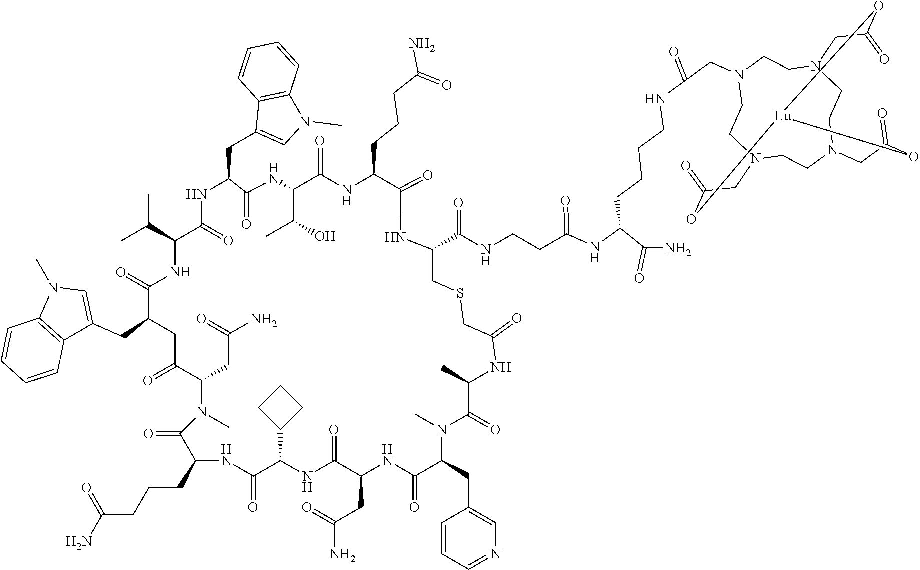 | HPLC-MS (Method A-B): 4.55 min ESI-MS m/z: 797.4 [M + 3H]3+ |
| PDC_EphA2- 00026631 (SEQ ID NO: 334) | 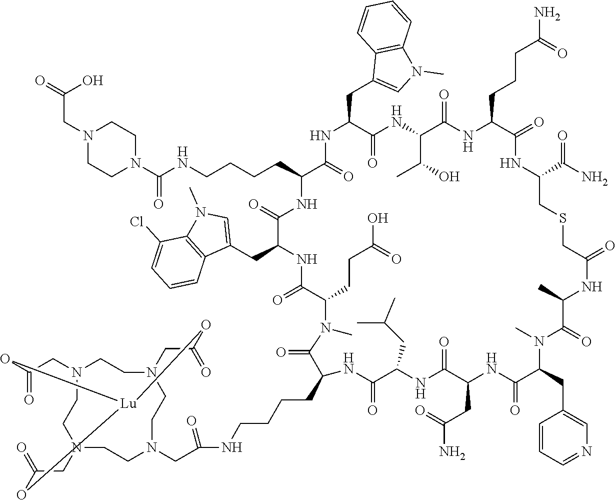 | HPLC-MS (Method A-B): 3.32 min ESI-MS m/z: 809.8 [M + 3H]3+ |
| PDC_EphA2- 00026632 (SEQ ID NO: 337) | 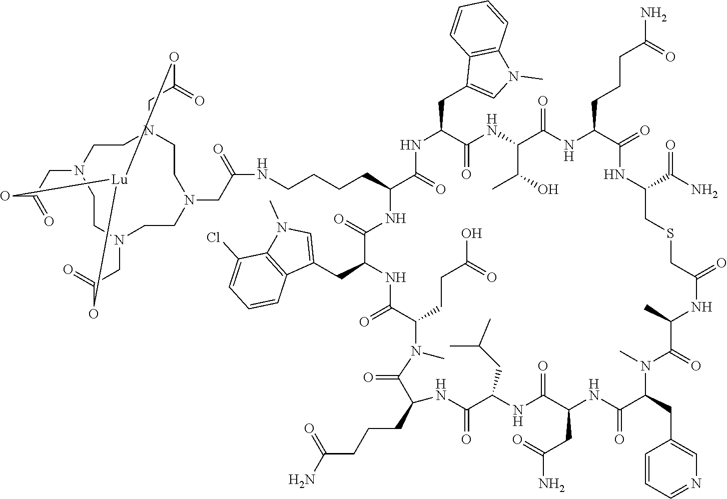 | HPLC-MS (Method A-B): 3.54 min ESI-MS m/z: 757.8 [M + 3H]3+ |
| PDC_EphA2- 00026633 (SEQ ID NO: 340) | 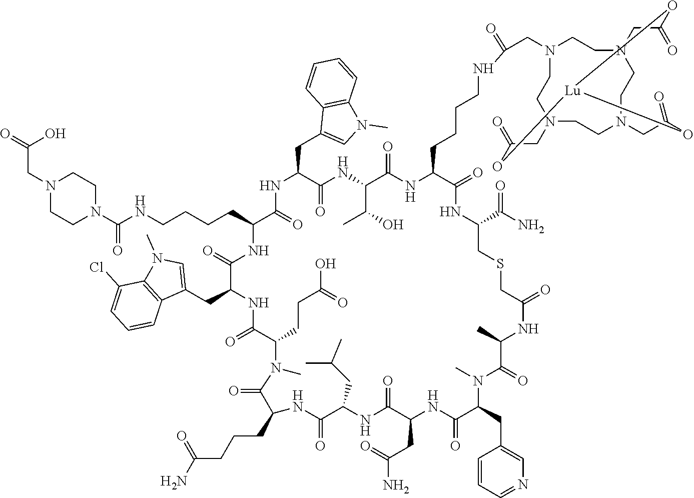 | HPLC-MS (Method A-B): 3.34 min ESI-MS m/z: 1214.3 [M + 2H]2+ |
| PDC_EphA2- 00026714 (SEQ ID NO: 335) | 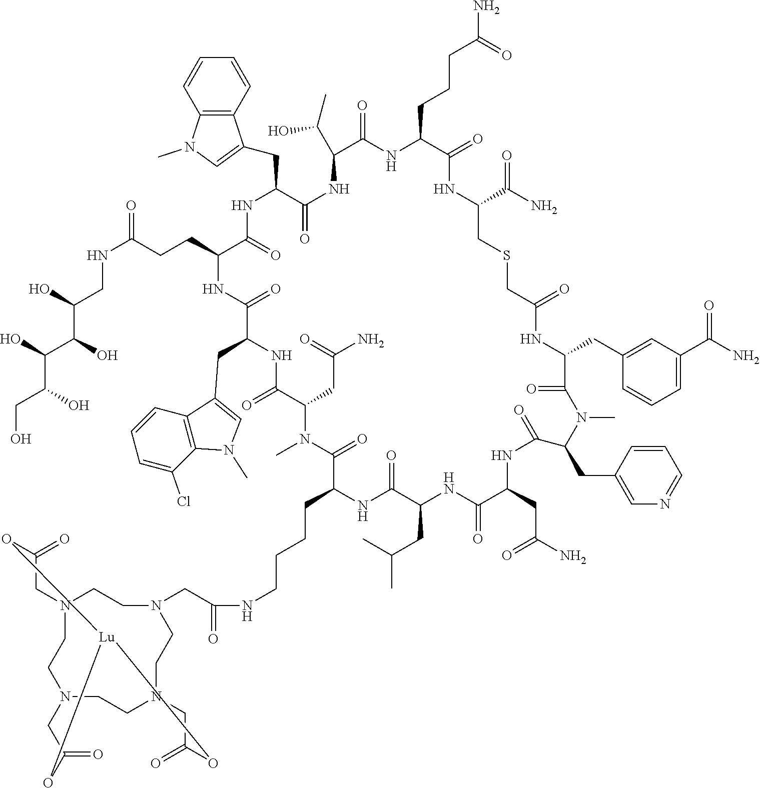 | HPLC-MS (Method A-B): 3.32 min ESI-MS m/z: 842.6 [M + 3H]3+ |
| PDC_EphA2- 00026715 (SEQ ID NO: 338) | 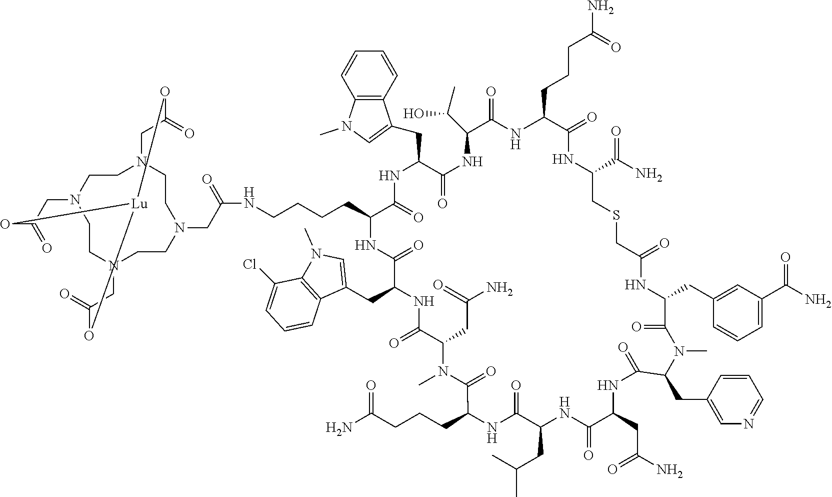 | HPLC-MS (Method A-B): 3.43 min ESI-MS m/z: 792.6 [M + 3H]3+ |
| PDC_EphA2- 00026716 (SEQ ID NO: 341) | 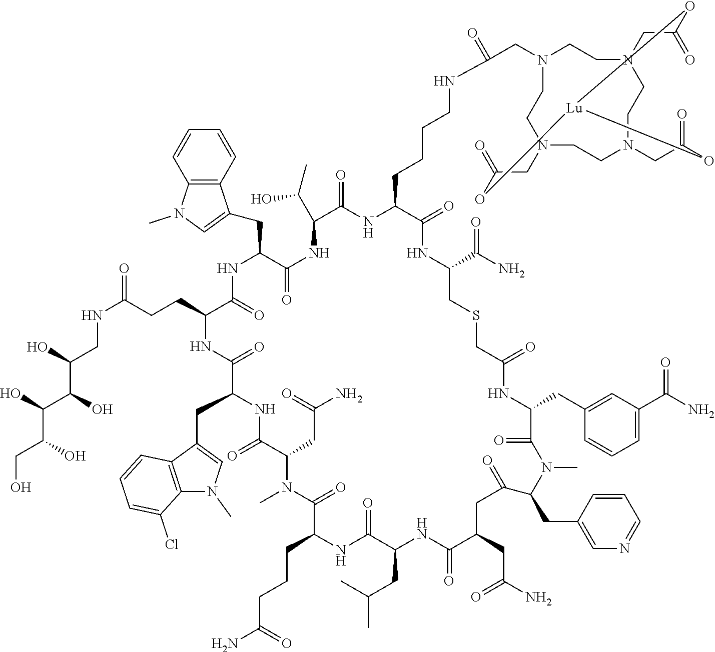 | HPLC-MS (Method A-B): 3.28 min ESI-MS m/z: 842.6 [M + 3H]3+ |
| PDC_EphA2- 00026717 (SEQ ID NO: 336) | 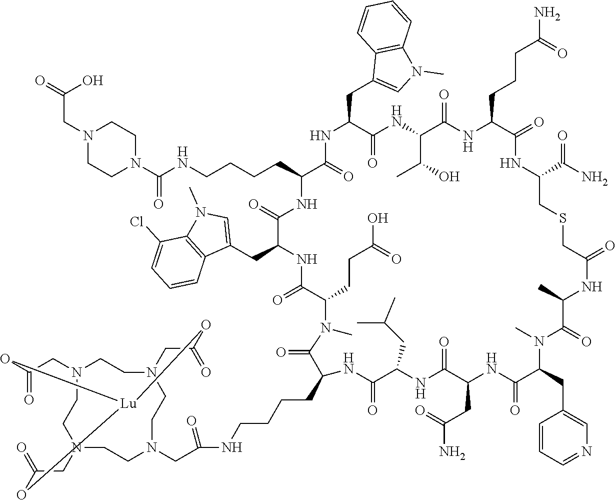 | HPLC-MS (Method A-B): 3.36 min ESI-MS m/z: 849.6 [M + 3H]3+ |
| PDC_EphA2- 00026718 (SEQ ID NO: 339) | 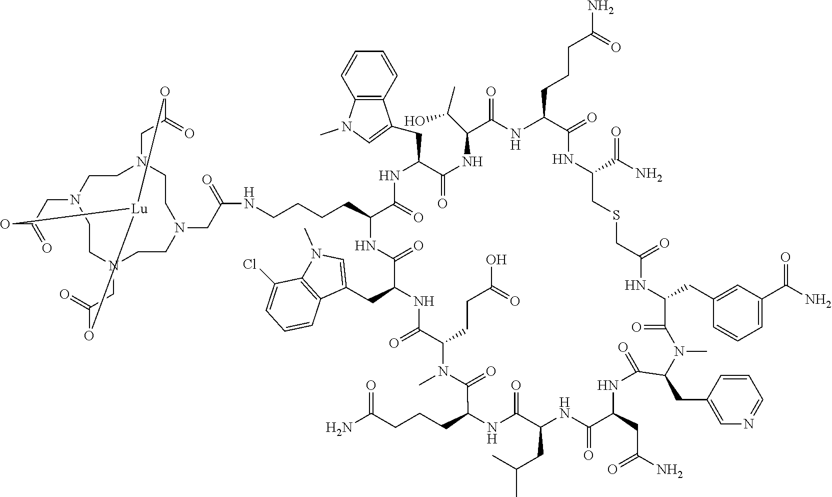 | HPLC-MS (Method A-B): 3.57 min ESI-MS m/z: 797.6 [M + 3H]3+ |
| PDC_EphA2- 00026719 (SEQ ID NO: 342) | 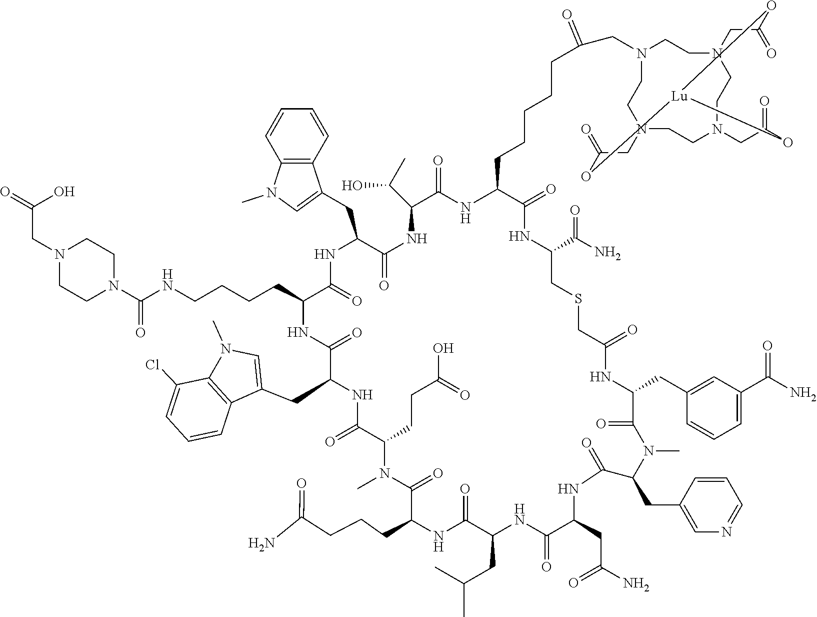 | HPLC-MS (Method A-B): 3.35 min ESI-MS m/z: 849.6 [M + 3H]3+ |
| PDC_EphA2- 00026772 (SEQ ID NO: 298) | 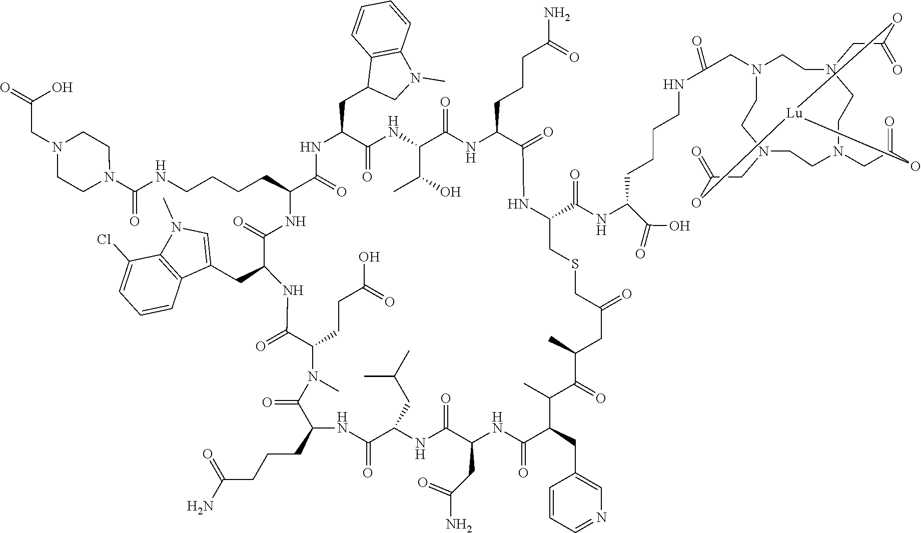 | HPLC-MS (Method A-B): 2.82 min ESI-MS m/z: 1268.6 [M + 2H]2+ |
| PDC_EphA2- 00026773 (SEQ ID NO: 316) | 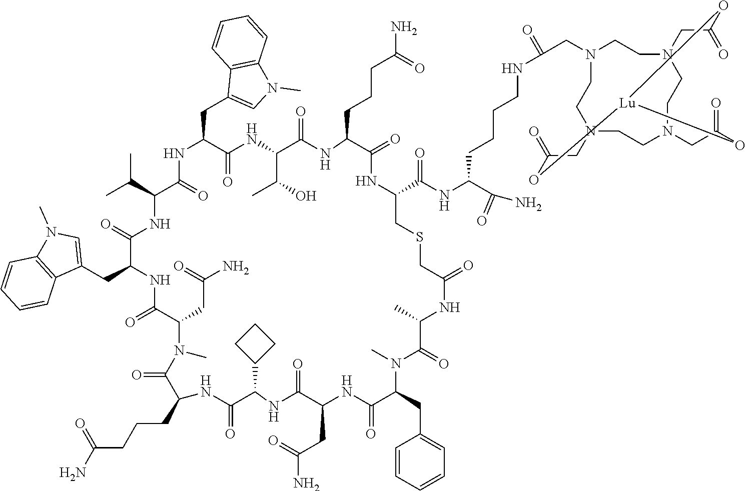 | HPLC-MS (Method A-B): 4.78 min ESI-MS m/z: 1160.0 [M + 2H]2+ |
| PDC_EphA2- 00026774 (SEQ ID NO: 324) | 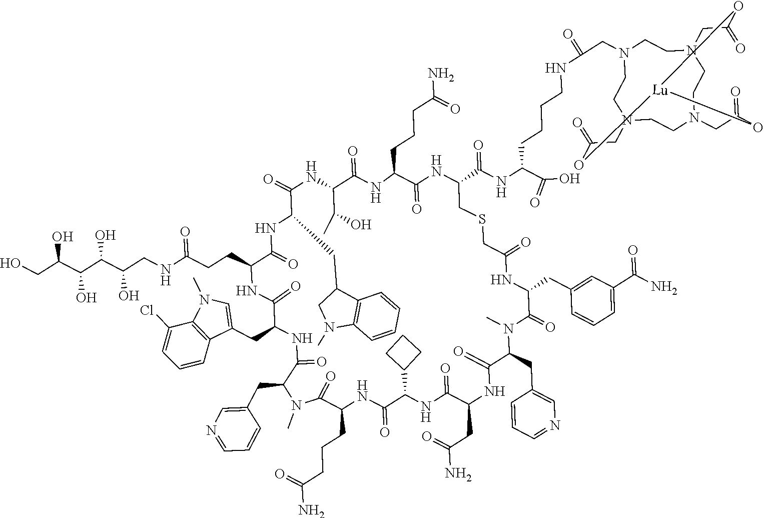 | HPLC-MS (Method A-B): 2.88 min ESI-MS m/z: 900.9 [M + 3H]3+ |
| PDC_EphA2- 00026775 (SEQ ID NO: 333) | 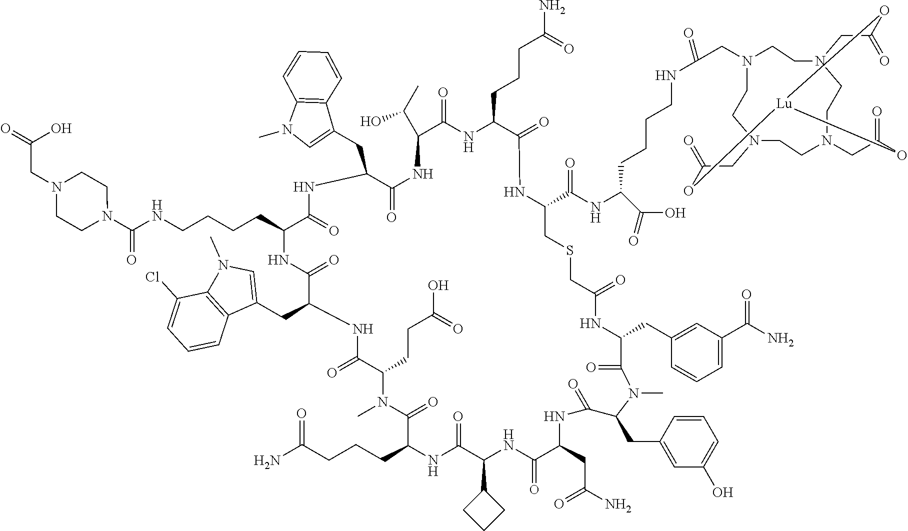 | HPLC-MS (Method A-B): 4.28 min ESI-MS m/z: 1351.9 [M + 2H]2+ |
| PDC_EphA2- 00001417- C304 (SEQ ID NO: 283) | 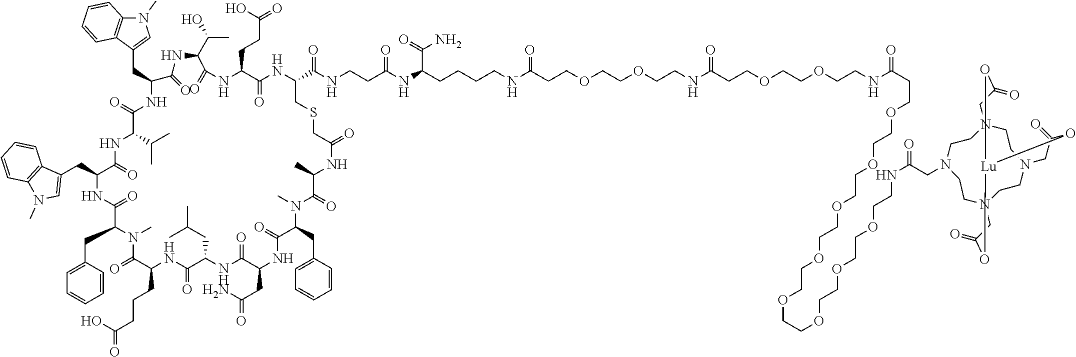 | HPLC-MS (Method A-B): 6.22 min ESI-MS m/z: 999.0 [M + 3H]3+ |
| PDC_EphA2- 00007196- C304 (SEQ ID NO: 290) | 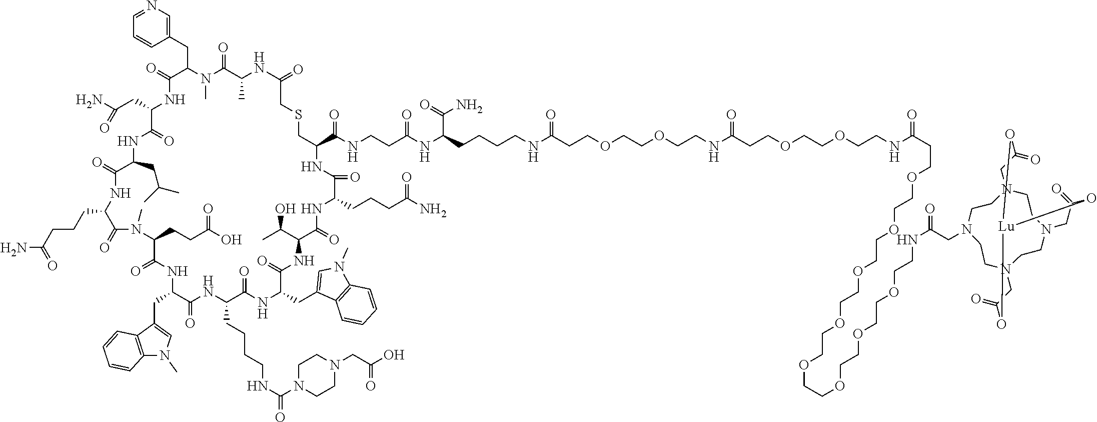 | HPLC-MS (Method A-B): 2.94 min ESI-MS m/z: 1063.6 [M + 3H]3+ |
| PDC_EphA2- 00008093- C304 (SEQ ID NO: 287) | 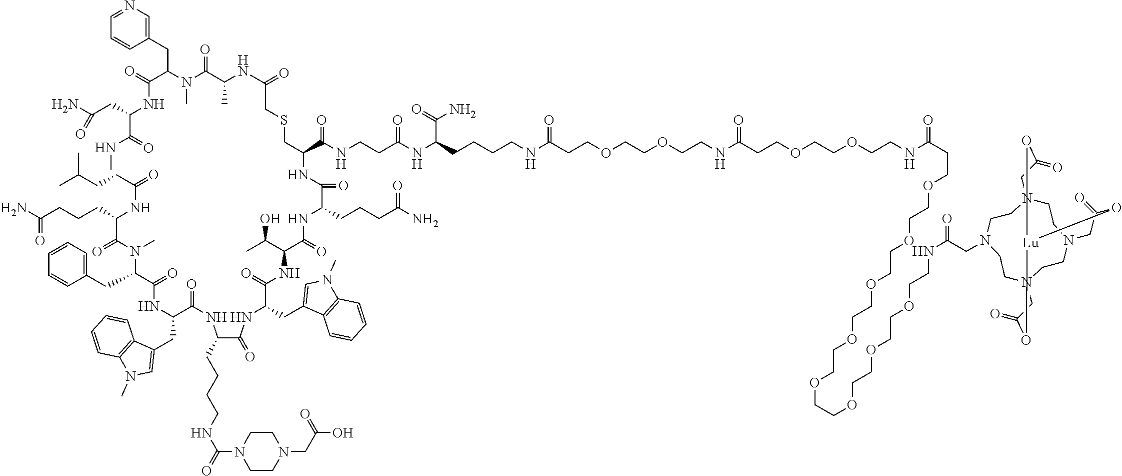 | HPLC-MS (Method A-B): 3.54 min ESI-MS m/z: 1069.7 [M + 3H]3+ |
| PDC_EphA2- 00008094- C304 (SEQ ID NO: 289) | 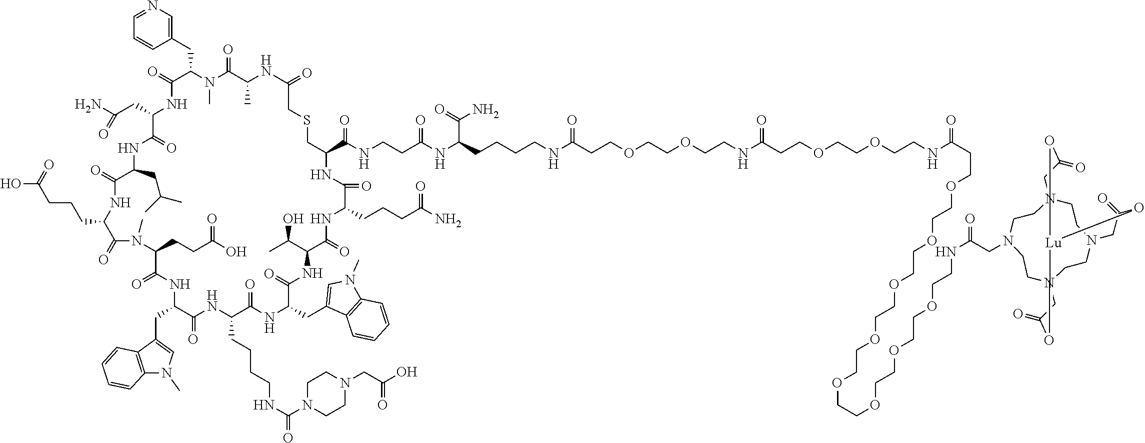 | HPLC-MS (Method A-B): 3.05 min ESI-MS m/z: 1064.0 [M + 3H]3+ |
| PDC_EphA2- 00010011- C304 (SEQ ID NO: 344) | 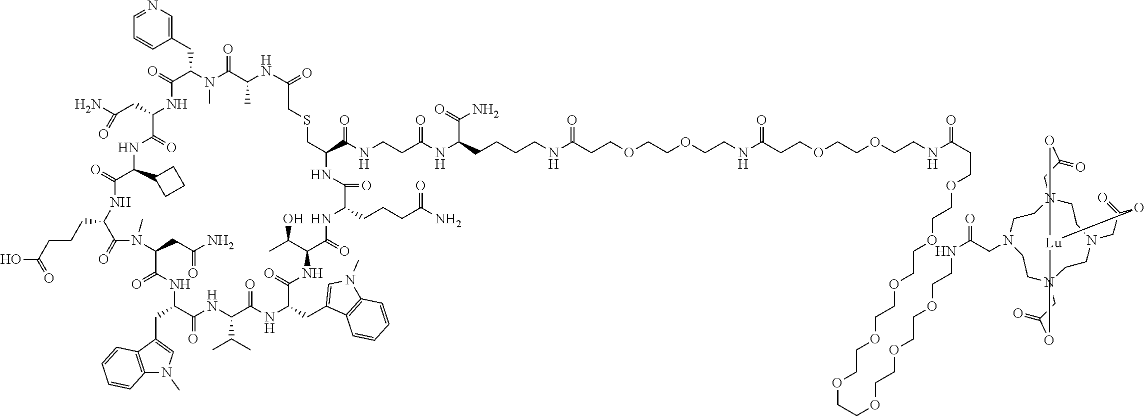 | HPLC-MS (Method A-B): 3.11 min ESI-MS m/z: 992.0 [M + 3H]3+ |
| PDC_EphA2- 00010012- C304 (SEQ ID NO: 346) | 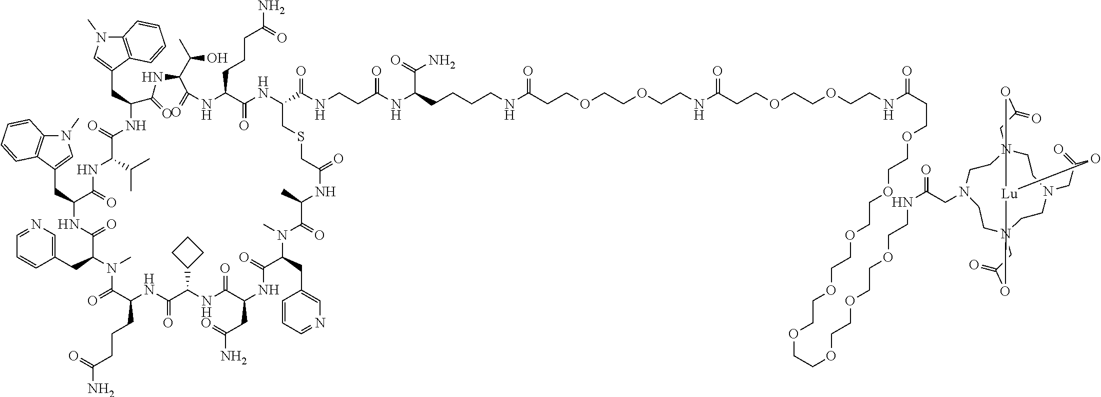 | HPLC-MS (Method A-B): 2.86 min ESI-MS m/z: 1003.3 [M + 3H]3+ |
| PDC_EphA2- 00013693- C304 (SEQ ID NO: 314) | 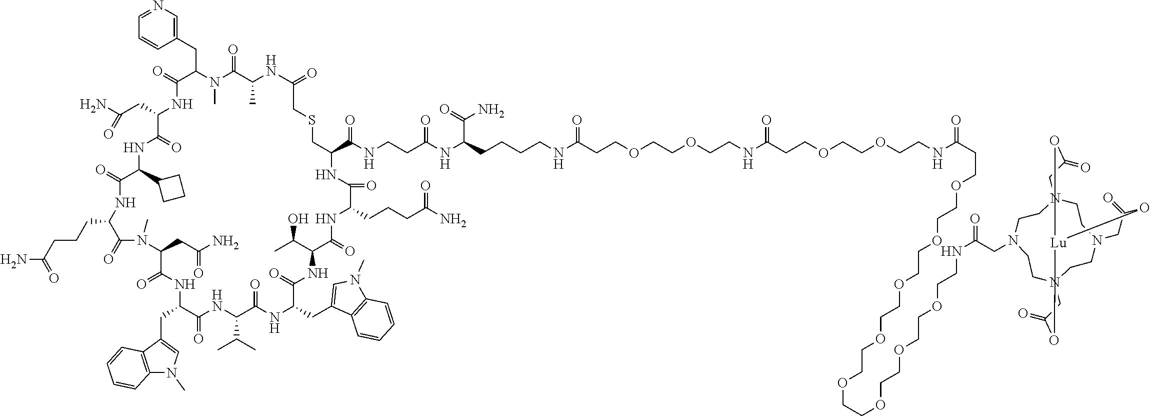 | HPLC-MS (Method A-B): 4.59 min ESI-MS m/z: 991.6 [M + 3H]3+ |
| PDC_EphA2- 00008093- C310 (SEQ ID NO: 286) | 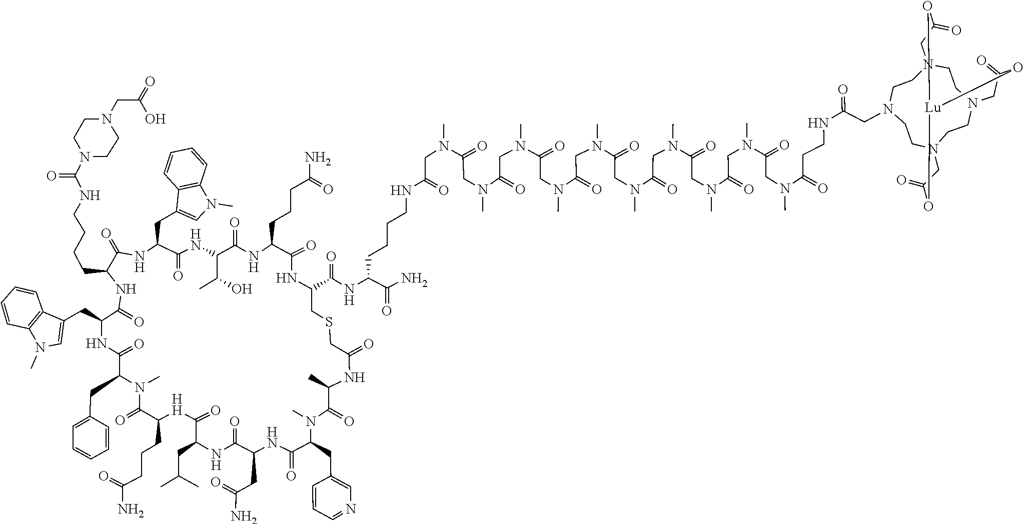 | HPLC-MS (Method A-B): 3.33 min ESI-MS m/z: 1112.4 [M + 3H]3+ |
| PDC_EphA2- 00007196- C310 (SEQ ID NO: 291) | 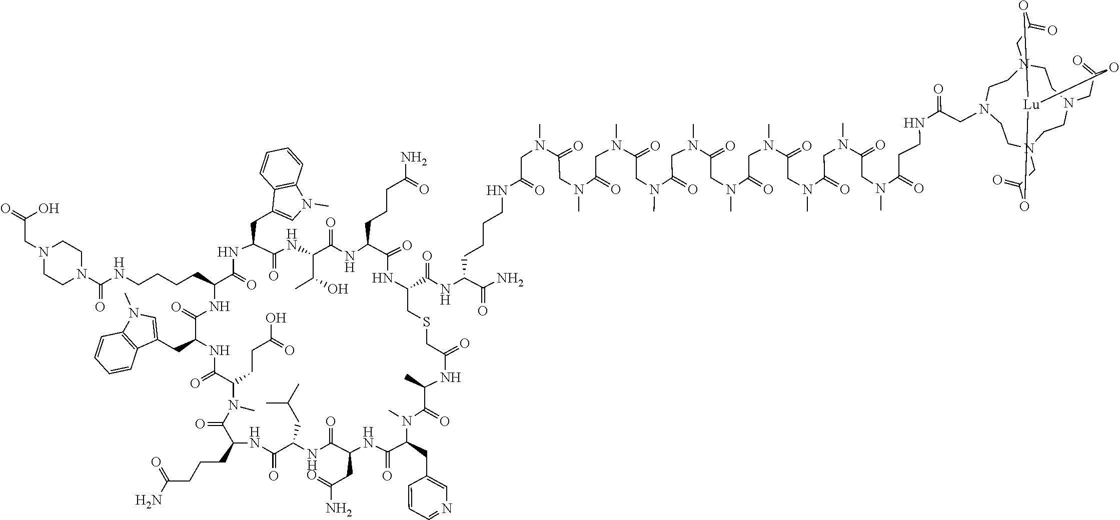 | HPLC-MS (Method A-A): 5.40 min ESI-MS m/z: 1106.3 [M+3H]3+ |
| PDC_EphA2- 00001417- C306 (SEQ ID NO: 282) | 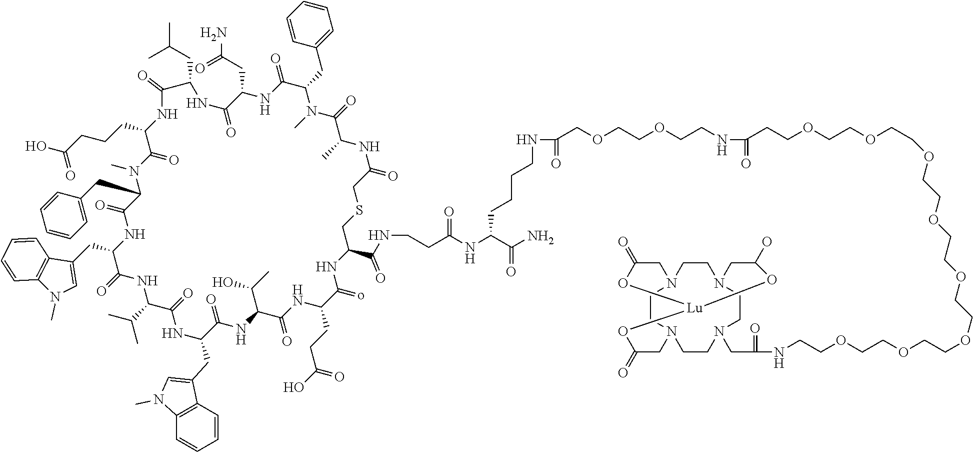 | |
| PDC_EphA2- 00019443- C302 (SEQ ID NO: 330) | 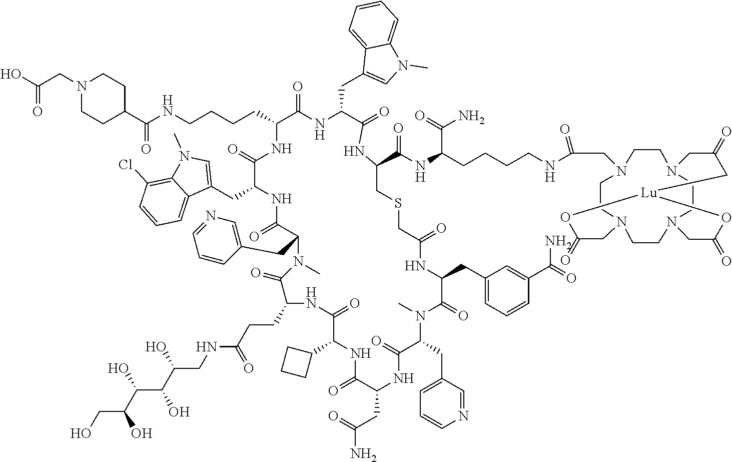 | |
| PDC_EphA2- 00008010- C302 (SEQ ID NO: 300) | 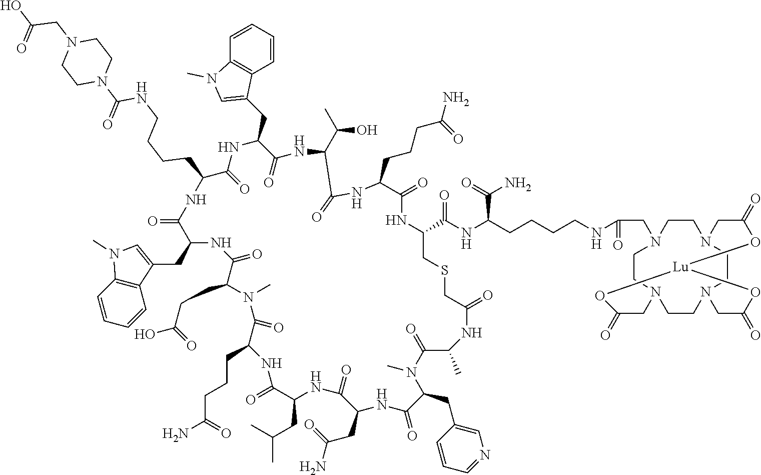 | |
| PDC_EphA2- 00026603- C301 (SEQ ID NO: 425) | 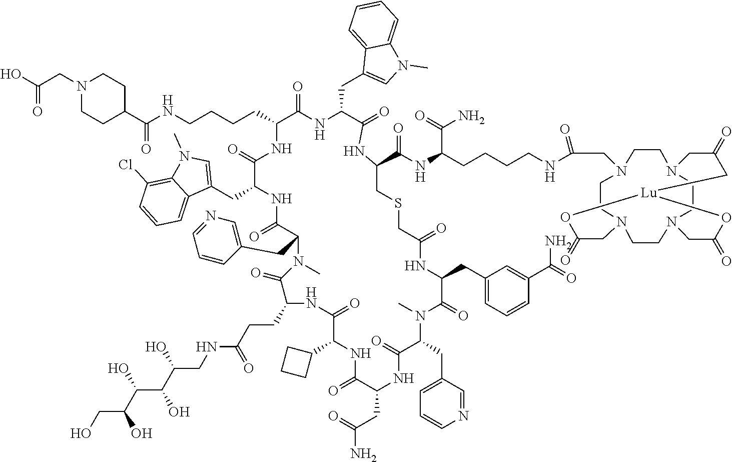 | |
| PDC_EphA2- 00007196- C306 (SEQ ID NO: 304) | 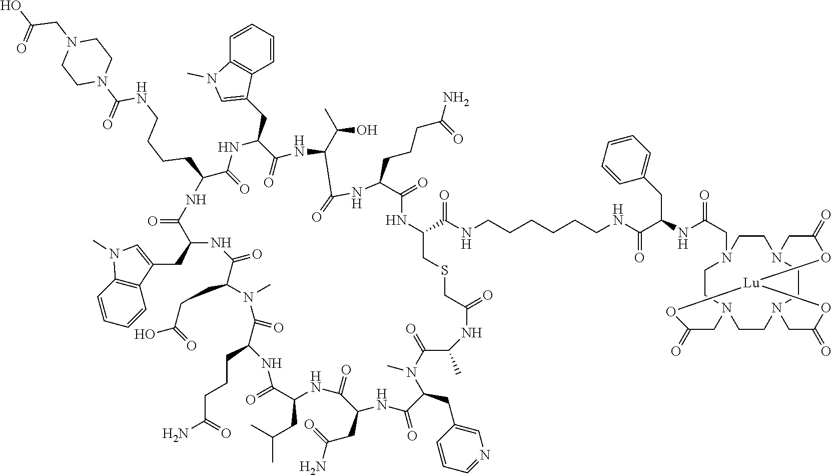 | |
| PDC_EphA2- 00007196- C307 (SEQ ID NO: 305) | 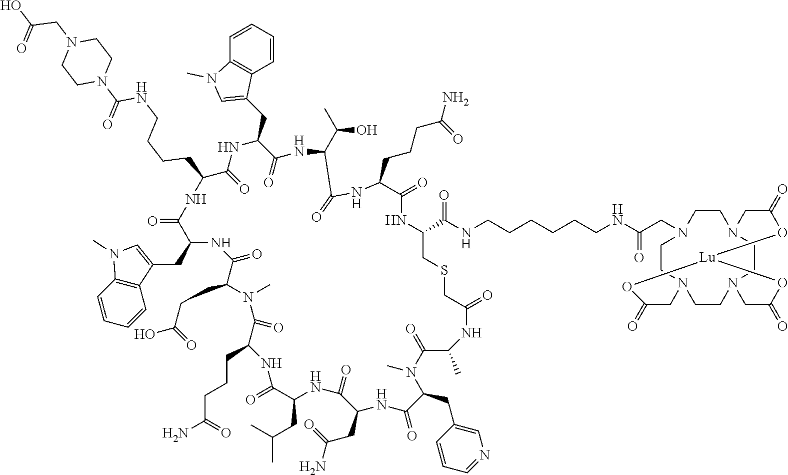 | |
| PDC_EphA2- 00007196- C308 (SEQ ID NO: 306) | 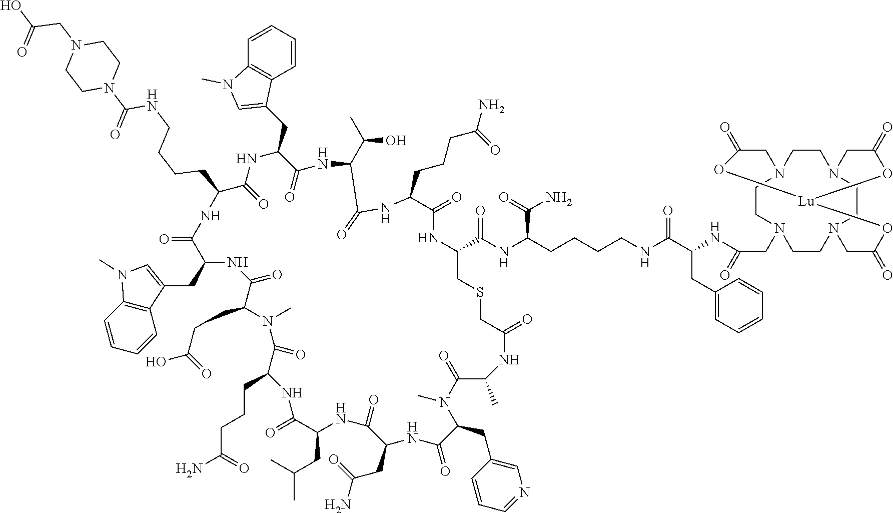 | |
| PDC_EphA2- 00007196- C309 (SEQ ID NO: 307) | 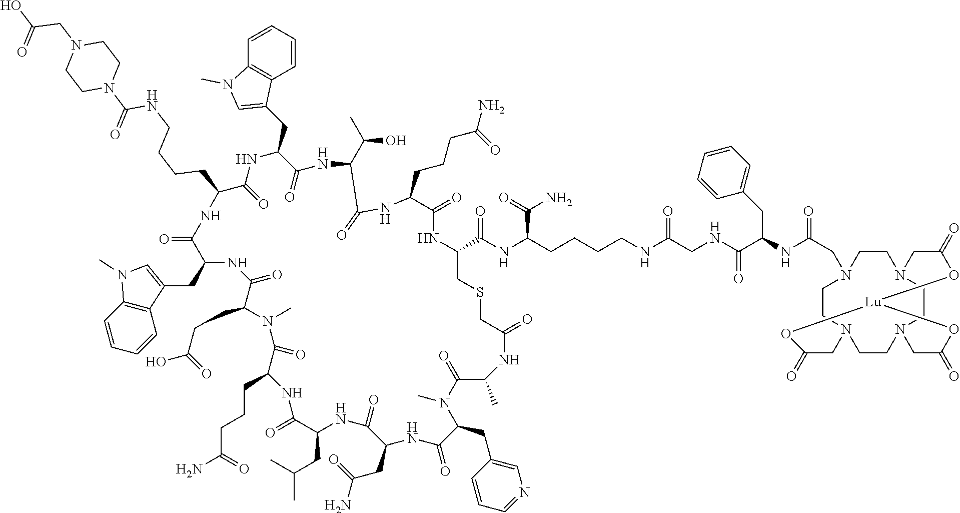 | |
| PDC_EphA2- 00027131 (SEQ ID NO: 308) | 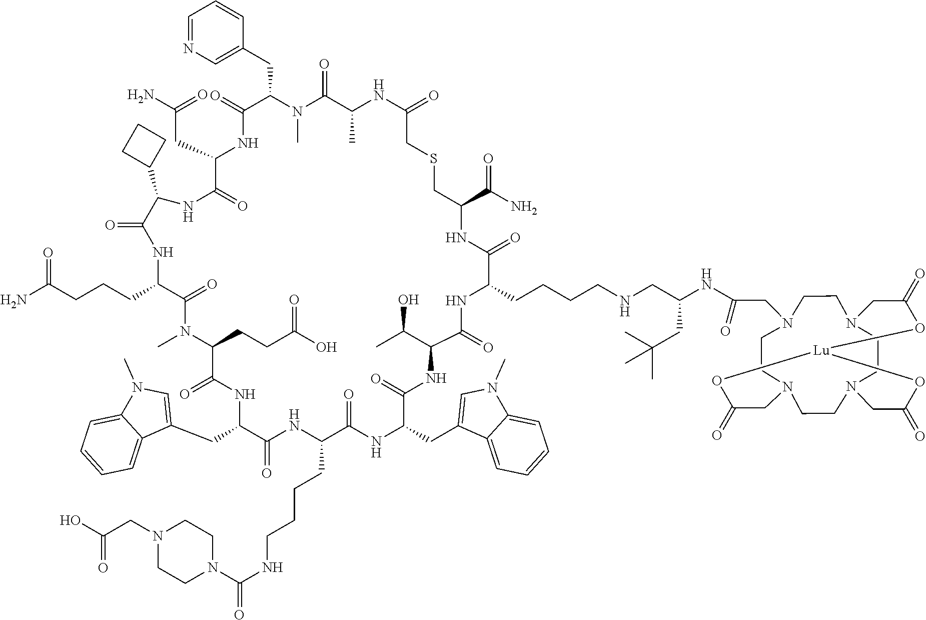 | |
| PDC_EphA2- 00027129 (SEQ ID NO: 309) | 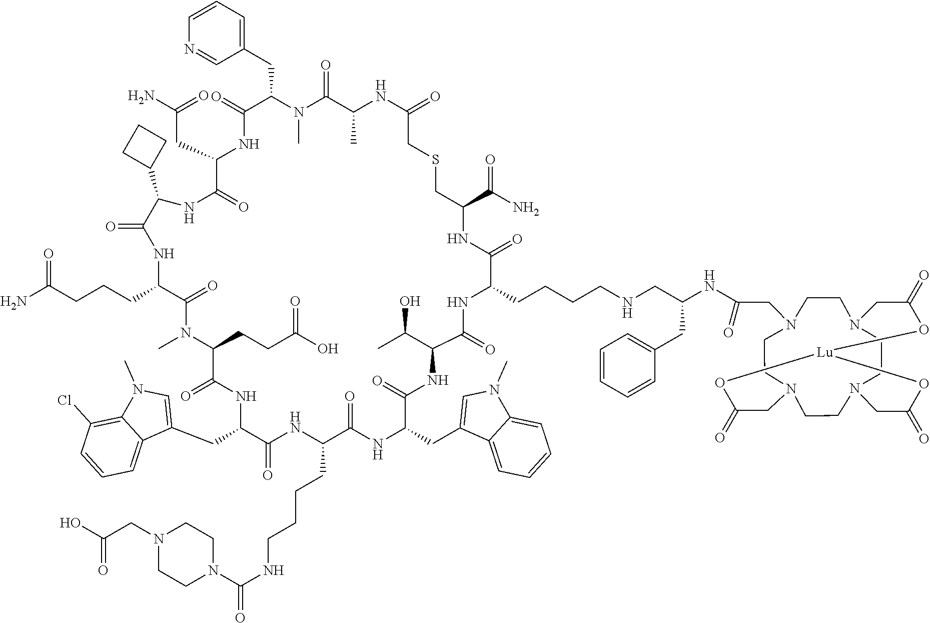 | |
| PDC_EphA2- 00027128 (SEQ ID NO: 310) | 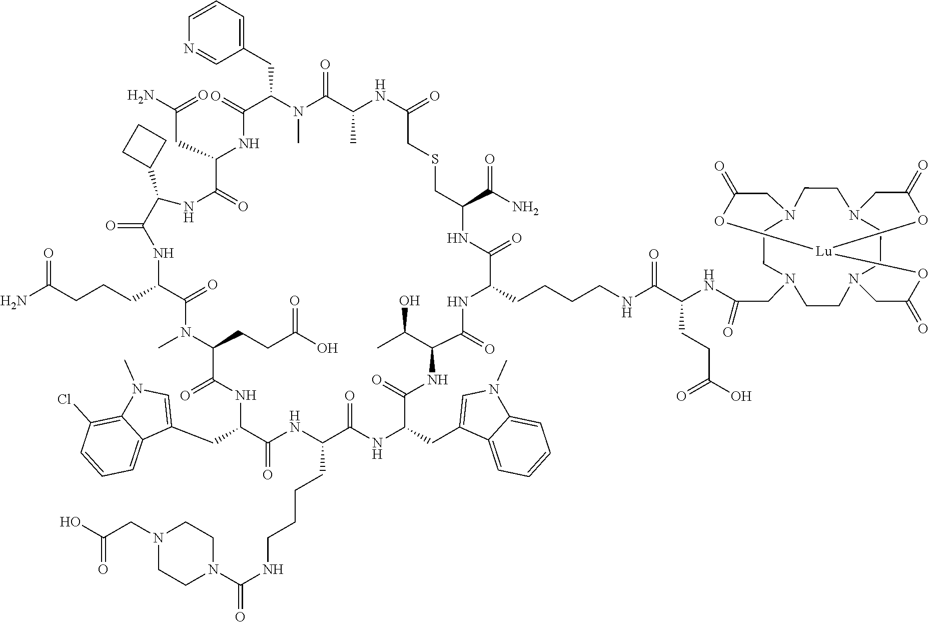 | |
| PDC_EphA2- 00027130 (SEQ ID NO: 311) | 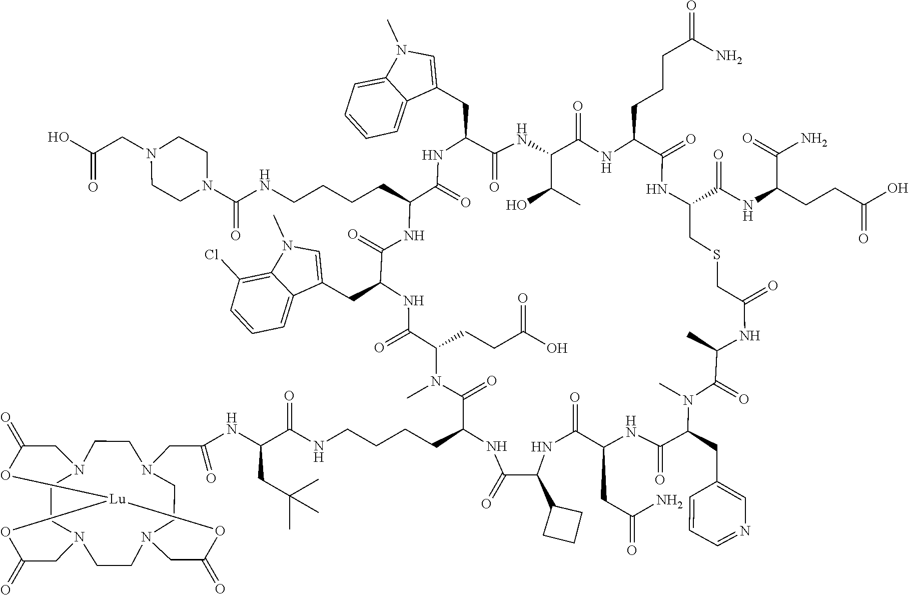 | |
| PDC_EphA2- 00027127 (SEQ ID NO: 312) | 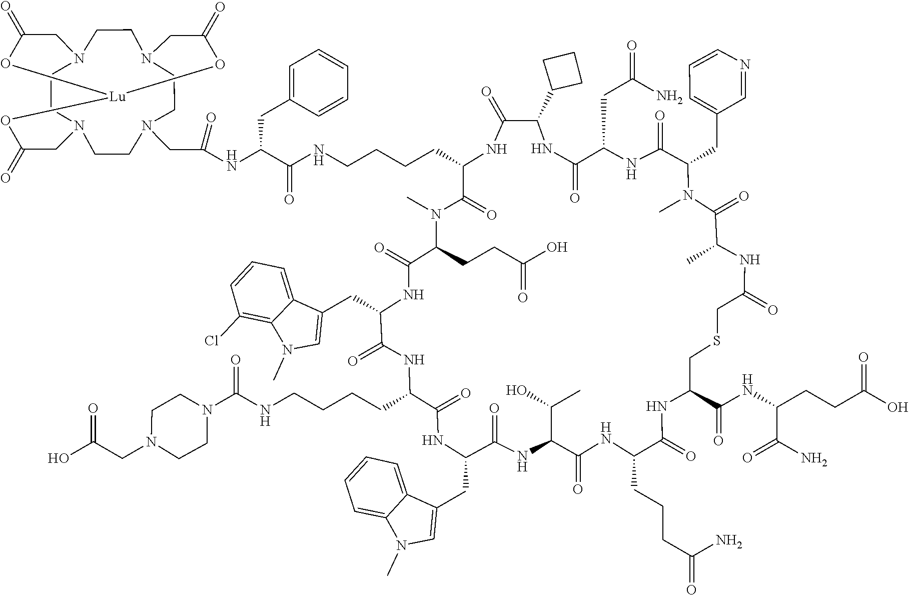 | |
| PDC_EphA2- 00027126 (SEQ ID NO: 313) | 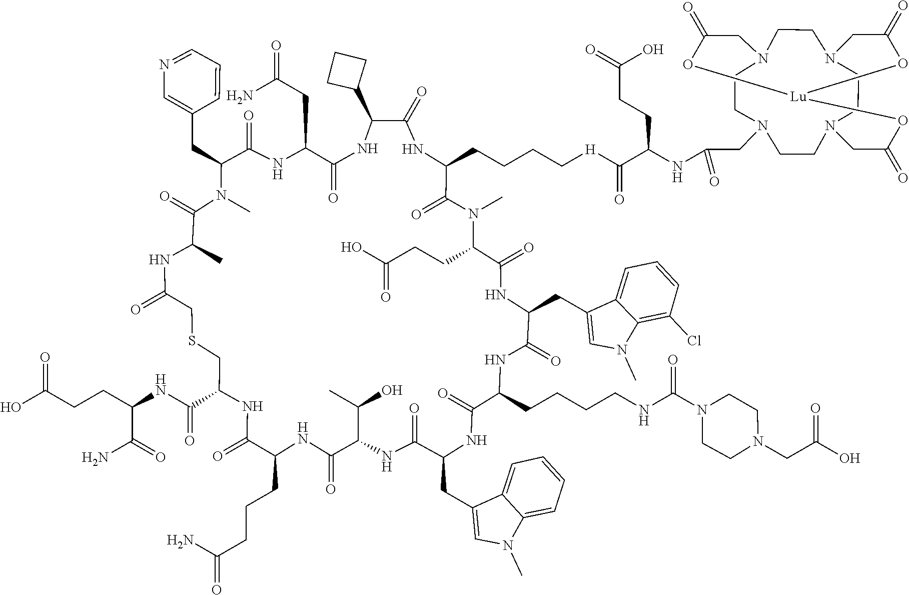 | |
| PDC_EphA2- 00027104 (SEQ ID NO: 356) | 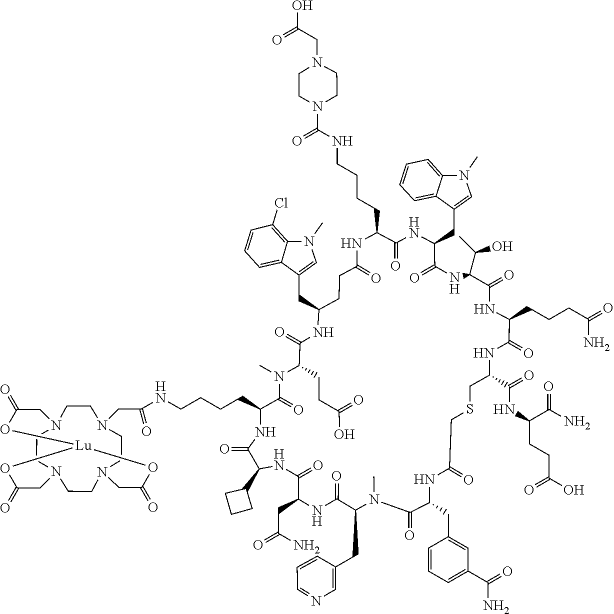 | |
| PDC_EphA2- 00027106 (SEQ ID NO: 357) | 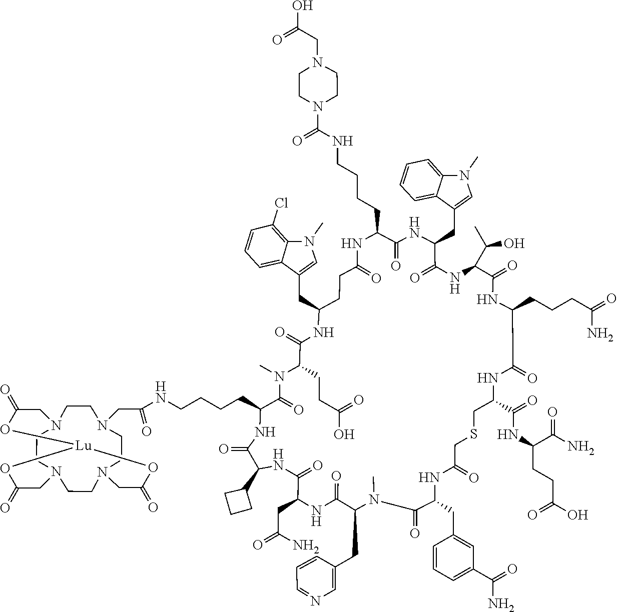 | |
| PDC_EphA2- 00027103 (SEQ ID NO: 358) | 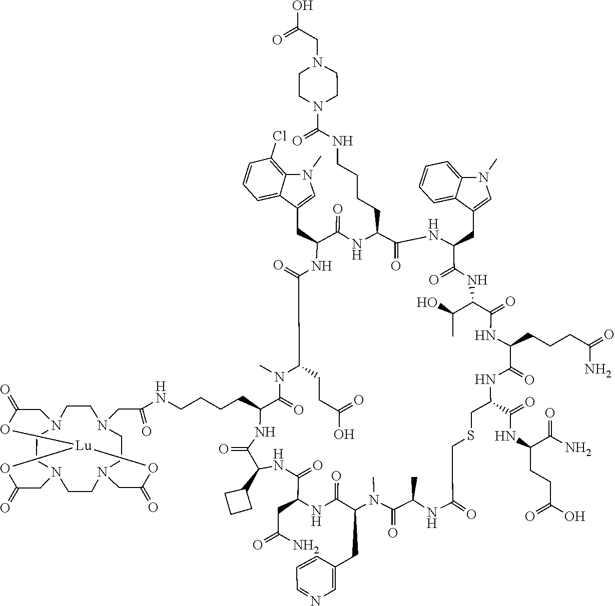 | |
| PDC_EphA2- 00027105 (SEQ ID NO: 359) | 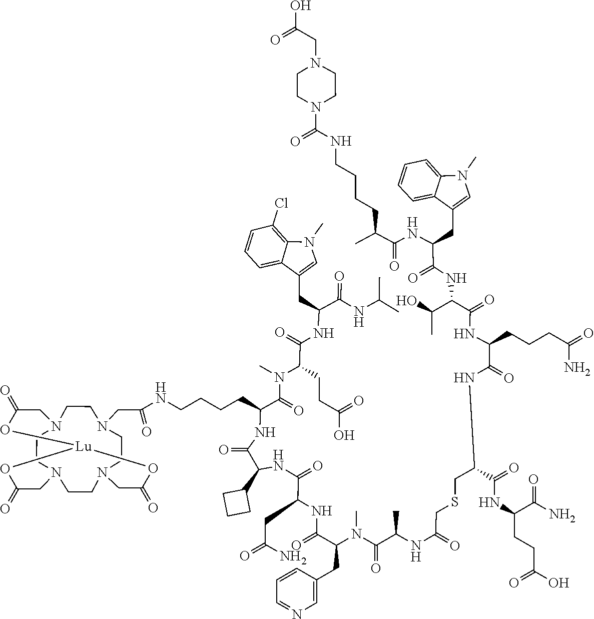 | |

### Example B20: PDC_EphA2-00026603-C201 (SEQ ID NOS 241 and 365, Respectively, in Order of Appearance)

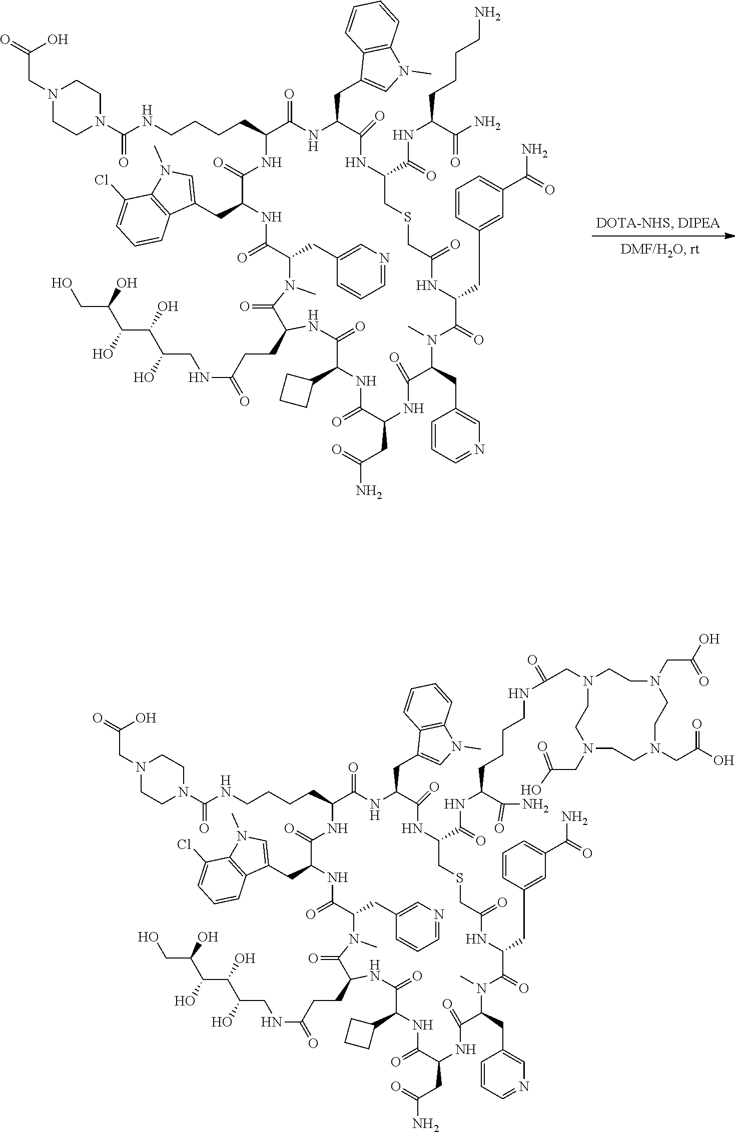

To a solution of PDC_EphA2-00026603-C001 (30 mg, 0.0130 mmol) in DMF (598 μL) at 0 °C were added DIPEA (18.79 μL, 0.108 mmol) and 0.12 M aq. DOTA-NHS ester (598 μL, 0.072 mmol). The mixture was stirred at rt for 0.5 h. Then to the mixture was added DIPEA (18.79 μL, 0.108 mmol) and stirred at rt for 0.5 h. The resulting residue was purified by preparative reverse phase C18-HPLC (Method P-A). Pure fractions were combined and lyophilized to afford the title compound. HPLC-MS (Method A-A): 5.20 min, ESI-MS m/z: 814.3 [M+3H]3+, UV-area (TIC): 97% Example B21

The following Examples were prepared by following similar methods described in the examples above. The amino acid sequence for each peptide was shown in Table B6 and Table B-61. The term “Term” means the functional group at C-terminus of the peptide.

TABLE B15

| Example | | |
|---|---|---|
| No. | Structure | Analytics |
| PDC_EphA2- 00026600- C301 (SEQ ID NO: 426) | 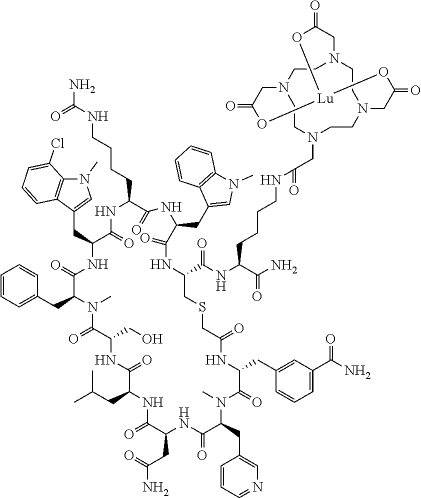 | HPLC-MS (Method A-B): 4.14 min ESI-MS m/z: 1141.7 [M + 2H]2+ |
| PDC_EphA2- 00026601- C301 (SEQ ID NO: 427) | 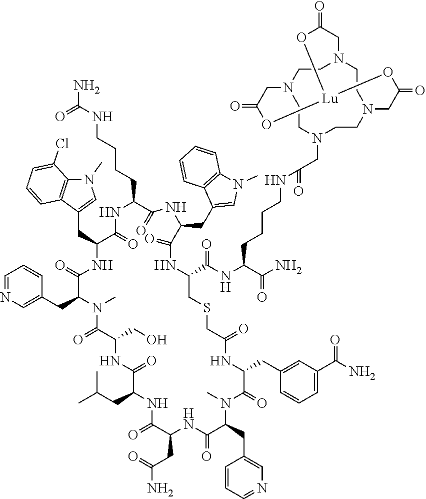 | HPLC-MS (Method A-B): 2.85 min ESI-MS m/z: 1142.2 [M + 2H]2+ |
| PDC_EphA2- 00026602- C301 (SEQ ID NO: 428) | 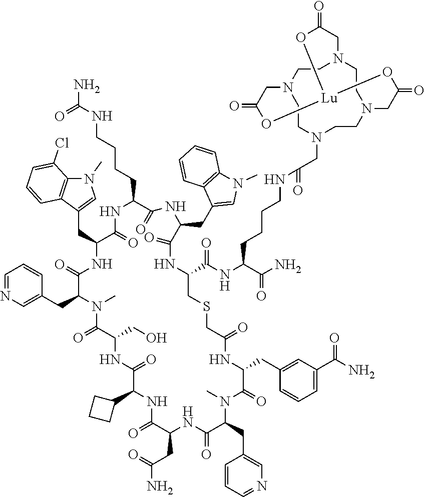 | HPLC-MS (Method A-A): 5.80 min ESI-MS m/z: 760.8 [M + 3H]3+ |
| PDC_EphA2- 00026604- C301 (SEQ ID NO: 429) | 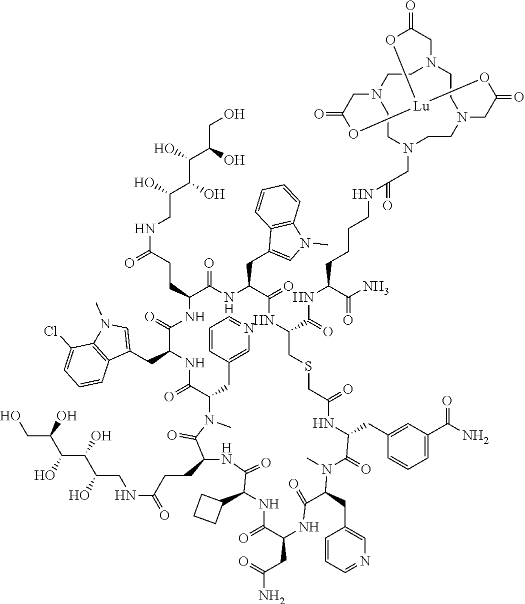 | HPLC-MS (Method A- A): 5.40 min ESI-MS m/z: 1303.8 [M + 2H]2+ |
| PDC_EphA2- 00026606- C301 (SEQ ID NO: 430) | 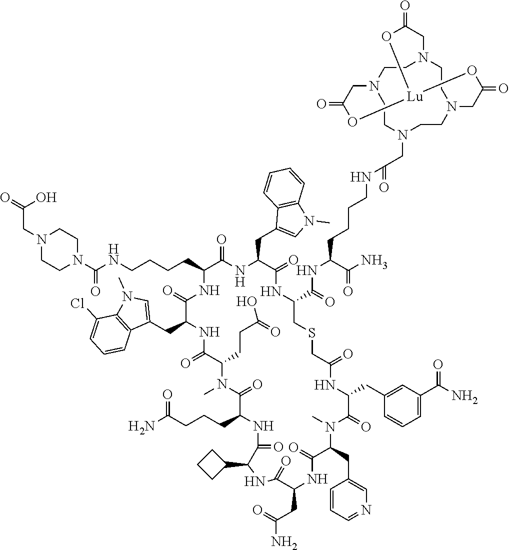 | HPLC-MS (Method A-B): 3.23 min ESI-MS m/z: 815.2 [M + 3H]3+ |
| PDC_EphA2- 00026607- C301 (SEQ ID NO: 431) | 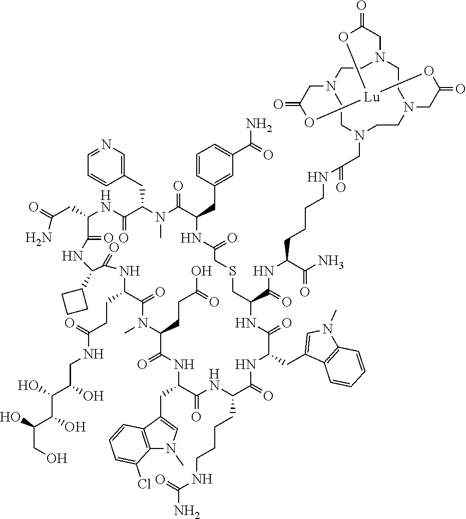 | HPLC-MS (Method A-B): 3.31 min ESI-MS m/z: 822.9 [M + 3H]3+ |
| PDC_EphA2- 00026608- C301 (SEQ ID NO: 432) | 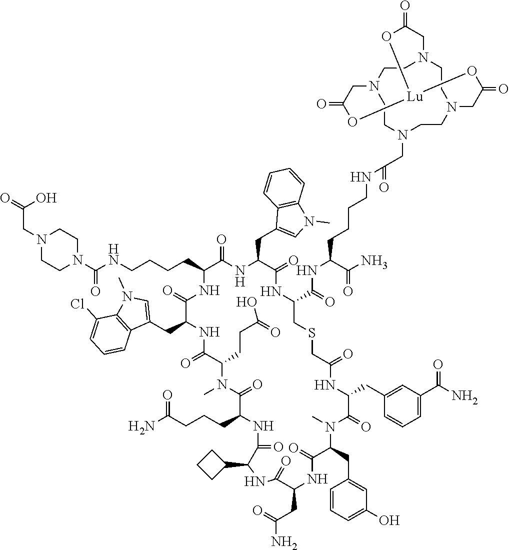 | HPLC-MS (Method A-B): 4.24 min ESI-MS m/z: 820.2 [M + 3H]3+ |

### Example B22: Interaction Analysis of Conjugates by SPR was Performed Using Biacore (Cytiva)

SPR assay was performed using Biacore T200 (Cytiva) according to Example B15. The result is shown in Table B16.

TABLE B16

| | SPR |
|---|---|
| Peptide ID | Kd (nM) |
| PDC_EphA2-00007196-C304 | A |
| PDC_EphA2-00008093-C304 | A |
| PDC_EphA2-00008094-C304 | A |
| PDC_EphA2-00008093-C302 | A |
| PDC_EphA2-00008093-C310 | A |
| PDC_EphA2-00007196-C302 | A |
| PDC_EphA2-00007196-C310 | A |
| PDC_EphA2-00013703-C302 | A |
| PDC_EphA2-00013704-C302 | A |
| PDC_EphA2-00013705-C302 | A |
| PDC_EphA2-00019437-C302 | A |
| PDC_EphA2-00022593-C302 | A |
| PDC_EphA2-00022594-C302 | A |
| PDC_EphA2-00022595-C302 | A |
| PDC_EphA2-00019440-C302 | A |
| PDC_EphA2-00022612-C302 | A |
| PDC_EphA2-00022613-C302 | A |
| PDC_EphA2-00022614-C302 | A |
| PDC_EphA2-00022615-C302 | A |
| PDC_EphA2-00026424-C302 | A |
| PDC_EphA2-00026425-C302 | A |
| PDC_EphA2-00026426-C302 | A |
| PDC_EphA2-00026631 | A |
| PDC_EphA2-00026632 | A |
| PDC_EphA2-00026633 | A |
| PDC_EphA2-00026714 | A |
| PDC_EphA2-00026715 | A |
| PDC_EphA2-00026716 | A |
| PDC_EphA2-00026717 | A |
| PDC_EphA2-00026718 | A |
| PDC_EphA2-00026719 | A |
| PDC_EphA2-00026772 | A |
| PDC_EphA2-00026773 | A |
| PDC_EphA2-00026774 | A |
| PDC_EphA2-00026775 | A |
| PDC_EphA2-00026600-C301 | A |
| PDC_EphA2-00026601-C301 | A |
| PDC_EphA2-00026602-C301 | A |
| PDC_EphA2-00026603-C301 | A |
| PDC_EphA2-00026604-C301 | A |
| PDC_EphA2-00026606-C301 | A |
| PDC_EphA2-00026607-C301 | A |
| PDC_EphA2-00026608-C301 | A |
| PDC_EphA2-00001417-C306 | B |
| PDC_EphA2-00001417-C304 | B |
| PDC_EphA2-00008010-C302 | A |
| PDC_EphA2-00019443-C302 | A |
| PDC_EphA2-00027131 | A |
| PDC_EphA2-00027129 | A |
| PDC_EphA2-00027128 | A |
| PDC_EphA2-00027130 | A |
| PDC_EphA2-00027127 | A |
| PDC_EphA2-00027126 | A |
| PDC_EphA2-00027104 | A |
| PDC_EphA2-00027106 | A |
| PDC_EphA2-00027103 | A |
| PDC_EphA2-00027105 | A |

SPR (Kd (nM)): 0 < A ≤ 1; 1 < B ≤ 10; 10 < C ≤ 100; 100 < D ≤ 400

### Example B23: Peptide Conjugates with Covalently Bound Radionuclides

Peptide conjugates of the present disclosure can be synthesized by following the step outlined in scheme I shown below according to Reaction A13-4.

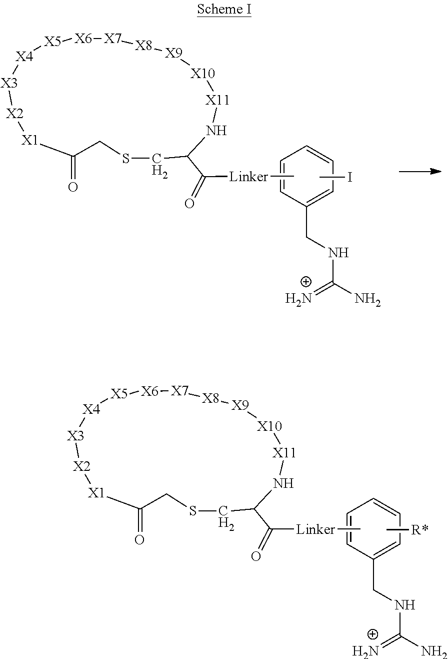

Covalently bound radionuclides can similarly be installed from alternative reactive intermediates according to Reaction A13-1, A13-2, A13-3, A13-5, A13-6, A13-7, A13-8, A13-9, and A13-10.

Peptide conjugates of the present disclosure can be synthesized by following the step outlined in scheme J shown below.

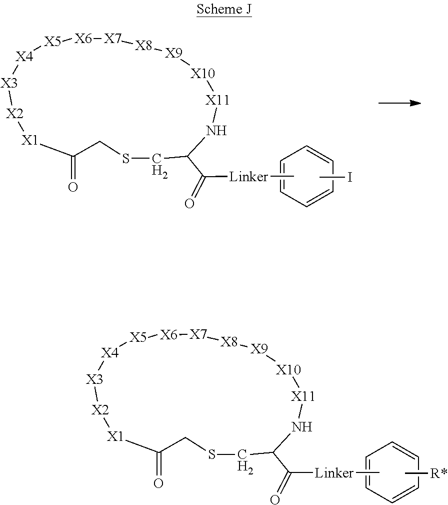

### C: Biological Assays.

### Example C1. Plasma Stability

The pooled frozen CD-1 (ICR) mouse plasma was thawed in a water bath at 37 °C prior to experiment. Plasma was centrifuged at 4000 rpm for 5 min and the clots were removed if any. pH was adjusted to 7.4±0.1 if required.

Plasma (98 μL) and test compounds (2 μL, 100 μM, in DMSO) or reference compound propantheline bromide (2 μL, 100 μM in 40% v/v MeOH/H2O) were added to the individual wells of a 96-well microtiter plate in duplicate. The plate was incubated at 37 °C. During the incubation, aliquots were withdrawn at 0, 10, 30, 60, 120, and 300 minutes. 100 μL 4% H₃PO₄ and 800 μL of stop solution (200 ng/mL tolbutamide and 200 ng/mL labetalol in 100% CH₃CN) were added to precipitate unwanted proteins. After mixing thoroughly, the quenched aliquots were centrifuged at 4,000 rpm for 20 min. Aliquots (100 μL) of supernatant were transferred to a new plate. The samples were shaken at 800 rpm for 10 min before performing LC-MS/MS analysis.

The analytes were detected by a multiple reaction monitoring method using a SCIEX Triple Quad 6500+ system equipped with an ACQUITY UPLC HSS T3 column (100 Å, 1.8 μm, 2.1 mm×30 mm). Mobile phase A: water with 0.1% formic acid; Mobile phase B: CH₃CN with 0.1% formic acid.

The % remaining of test compound after incubation in plasma was calculated using the following equation:

% Remaining=100×(PAR(t)/PAR(t0)), where PAR is the peak area ratio of analyte versus internal standard (IS)

TABLE C1
Plasma Stability

| Compound | Time Point | % Remaining | | T_{1/2} (min) | |
|---|---|---|---|---|---|
| ID | (min) | Mouse | Human | Mouse | Human |
| PDC_EphA2- | 0 | 100.0 | 100.0 | >289 | >289 |
| 00007196- | 120 | 99.4 | 96.3 | | |
| C302 | | | | | |
| PDC_EphA2- | 0 | 100.0 | 100.0 | >289 | >289 |
| 00008093- | 120 | 101.2 | 101.9 | | |
| C302 | | | | | |
| PDC_EphA2- | 0 | 100.0 | 100.0 | >289 | >289 |
| 00008093- | 120 | 106.8 | 99.0 | | |
| C310 | | | | | |
| PDC_EphA2- | 0 | 100 | 100 | >289.1 | >289 |
| 00001417- | 120 | 91 | 99 | | |
| C306 | | | | | |

### Example C2. In Vivo Pharmacokinetic Studies in Female CD-1 (ICR) Mice

The pharmacokinetics of peptide are determined using male CD-1 (ICR) mice purchased from Vital River Laboratory Animal Co., Ltd., Beijing, China. All animal studies are conducted in accordance with the highest standards of care as outlined in the NIH Guide for Care and Use of Laboratory. Following injection of the mice (10 mg/kg, 3 mice per test compound) with aliquots of the peptides in 10 mM PBS (2 mg/mL, pH 7.4) via the tail vein, blood samples are collected into pre-chilled tubes containing Heparin-Na (3 μL, 1,000 I.U./mL) at 5, 30, 60, and 240 min.

General sample processing procedure: An aliquot of 12 μL diluted blood sample (10× dilution factor for 30, 60, and 240 min blood samples; 20× dilution factor for 5 min blood samples), calibration standard, dilution quality control, single blank or double blank samples are added to the individual wells of a low binding 96-well plate. Each sample (except the double blank) is quenched with 120 μL MeOH. The resulting mixtures are mixed for 10 min at 800 rpm and centrifuged at 3,220 g (4000 rpm) for 15 min at 4 °C. Supernatant aliquots (50 μL) are transferred to a clean low binding 96-well plate and centrifuged at 3,220 g (4000 rpm) for 5 min at 4 °C, then the samples are directly injected for LC-MS/MS analysis.

The analytes are detected by a multiple reaction monitoring method using a SCIEX Triple Quad 6500+ system equipped with an ACQUITY UPLC HSS T3 column (100 Å, 1.8 μm, 2.1 mm×30 mm). Mobile phase A: water/CH₃CN (95/5, v/v) with 0.1% formic acid and 2 mM ammonium formate; Mobile phase B: CH₃CN/water (95/5, v/v) with 0.1% formic acid and 2 mM ammonium formate. Or Mobile phase A: water with 0.1% formic acid; Mobile phase B: CH₃CN with 0.1% formic acid. Column temperature: 60 °C

The plasma concentration-time data is subjected to IV-noncompartmental pharmacokinetics analysis using Phoenix WinNonlin (version 6.3, Pharsight Corp., Mountain View, CA, USA). The linear/log trapezoidal rule is applied in obtaining the PK parameters.

### Example C3. In Vitro Determination of Bound and Unbound Fraction of EphA2 Binding Constructs to Human Serum Albumin (HSA)

### HSA-HPLC Method (Measurement of Drug Protein Binding by Immobilized Human Serum Albumin-HPLC).

A 13-min HPLC (Thermo Vanquish Horizon with Diode Array Detector) gradient method is used to determine the HSA (Human Serum Albumin) binding of novel compounds using a chemically bonded protein stationary phase (ChiralPAK HSA HPLC column, 50×4 mm). The HSA binding values are derived from the gradient retention times that are converted to the logarithm of the equilibrium constant using data from a calibration set of molecules. The % bound to plasma values for the calibrator compounds are converted to the linear free energy values using the following equation: Log K=log[% PPB/(101-% PPB)]. The logarithmic value of the gradient retention times from the experiment are plotted against the linearized values of the % bound to plasma. The slope and the intercept are used to convert the retention times to linear free energy values (Log K), from which the estimated % protein binding is calculated using the following equation: % Binding=[(101·10^{Log K})/(1+10^{Log K})]. Aqueous mobile phase (mobile phase A) is 50 mM ammonium acetate solution, pH 7.4 and the organic mobile phase (mobile phase B) is 2-propanol. The flow rate is set at 0.35 mL/min and injection volume was 5 μL, with samples prepared at 0.5 mg/mL concentration in 50:50 mobile phase. The initial LC conditions are set at 0% B and ramped to 50% B over 8.5 min, then held at 50% B for 1.5 min before going back to initial conditions and re-equilibrating the column for 2.5 min. Chromatograms are recorded at 280 nm by a diode array UV absorption detector.

### Example C4. Protocol-FACS Analysis for Biotinylated Compound Cell Binding

Cells were split, counted, and resuspended in cold PBS. Cells were stained with Zombie Violet dye (1:1000 in PBS) at dark for 15 min. Cells were washed two times with cold PBS by centrifugation (@1,500 RPM for 3 min). A total of 50K cells per 90 μL cell culture media were seeded in each well of a 96 wells of U bottom plate. 10 μL of serial diluted compounds were added to 90 μL of cells in each well. For biotinylated compounds in direct binding experiments: 3-fold of serial diluted biotinylated compounds are prepared with DMSO in a separate plate prior to adding to cells. Cells were incubated with compounds on ice for 1 hour for binding. Cells were then washed 3 times with cold PBS by centrifugation (@1,500 RPM for 3 min). Compounds bound cells were then stained with Streptavidin-AF647 (1:2000 dilution in PBS) on ice for 30 minutes. Cells were washed 3 times with cold PBS by centrifugation (@1,500 RPM for 3 minutes). Cells were resuspended in 100 μL of cold PBS and proceeded to APC signal acquisition on CytoFlex.

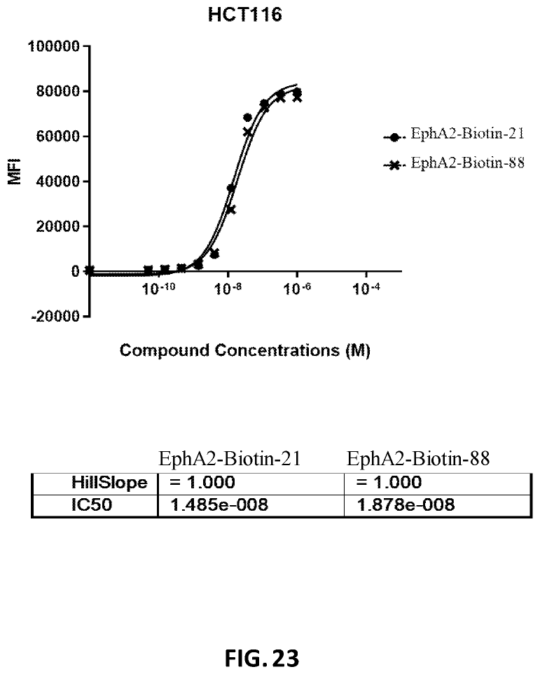

Cell binding of biotinylated compounds EphA2-Biotin-21 and EphA2-Biotin-88 were tested in HCT116 cells and binding EC50 were calculated by GraphPad Prism 9.1, see FIG. 23.

EphA2-Biotin-21 has a structure below (both disclosed as SEQ ID NO: 422), wherein the da at residue position 1 is connected with C at residue position 12, and the linker-biotin (hA-dk(acea-PEG8-Biotin)) is attached to the C at residue position 12.

| 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|
| da | MeF | NL | | Hgl | MeF | W1Me | V | W1Me | T | E | C | bA | dk(aeea- | —NH2 |
| | | | | | | | | | | | | | PEG8- | |
| | | | | | | | | | | | | | Biotin) | |

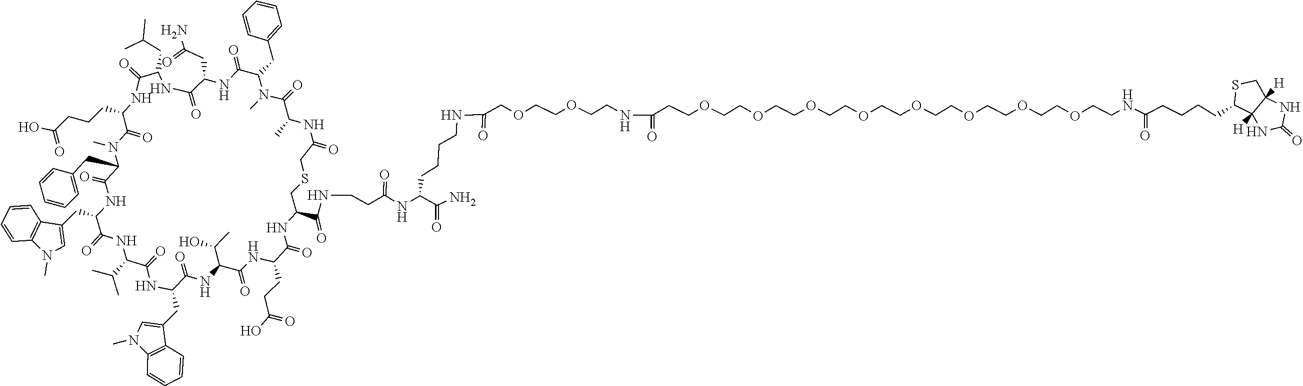

EphA2-Biotin-88 has a structure below (both disclosed as SEQ ID NO: 423), wherein the da at residue position 1 is connected with C at residue position 12, and the linker-biotin (G-PEG10-K(Biotin)) is attached to the C at residue position 12.

| 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|
| da | Me3Py | N | L | Hgn | MeE | W1Me | KCOpipzaa | W1Me | T | Hgn | C | G | PEG10 | K(Biotin) |

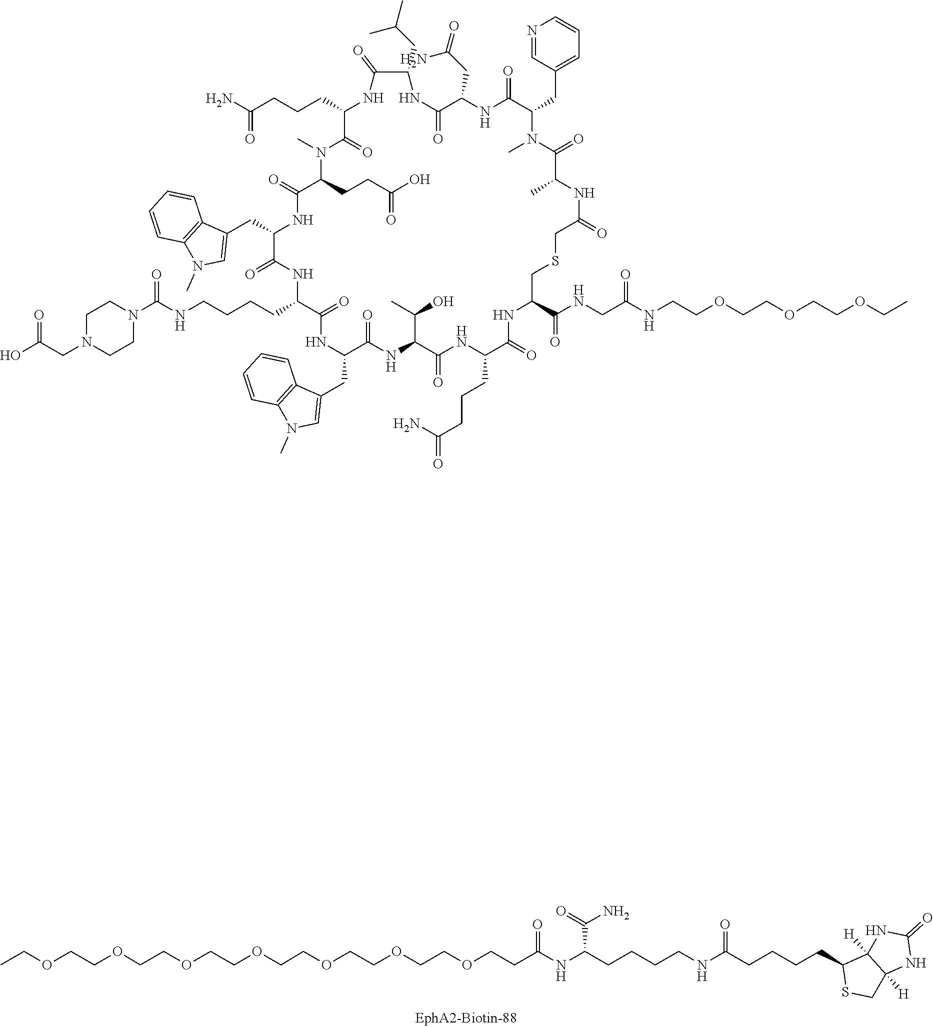

### Example C5. Protocol-FACS Analysis for Non-Biotinylated Compound Cell Competition Binding

Cells were split, counted, and resuspended in cold PBS. Cells were stained with Zombie Violet dye (1:1000 in PBS) at dark for 15 min. Cells were washed two times with cold PBS by centrifugation (@1,500 RPM for 3 min). A total of 50K cells per 90 μL cell culture media were seeded in each well of a 96 wells of U bottom plate. 10 μL of serial diluted compounds were added to 90 μL of cells in each well. For non-biotinylated compounds in competition binding experiments: 3-fold of serial diluted non-biotinylated compounds are prepared with 10 nM of biotinylated compounds in a separate plate prior to adding to cells. Cells were incubated with compounds on ice for 1 hour for binding. Cells were then washed 3 times with cold PBS by centrifugation (@1,500 RPM for 3 min). Compound bound cells were then stained with Streptavidin-AF647 (1:2000 dilution in PBS) on ice for 30 minutes. Cells were washed 3 times with cold PBS by centrifugation (@1,500 RPM for 3 min). Cells were resuspended in 100 μL of cold PBS and proceeded to APC signal acquisition on CytoFlex (Beckman Coulter Life Sciences, Indianapolis, IN).

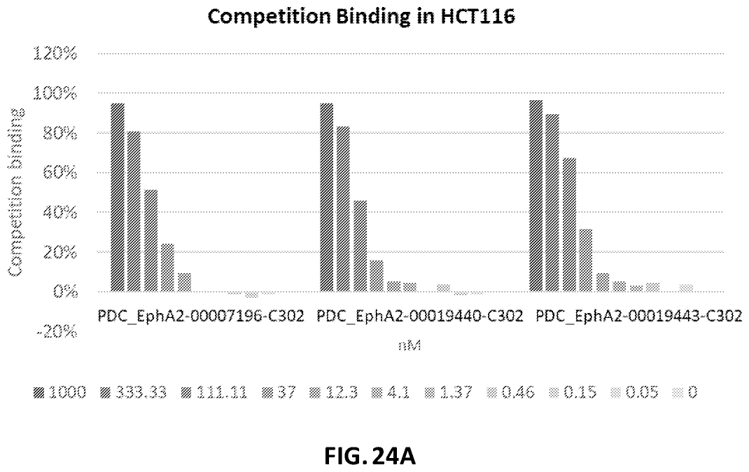

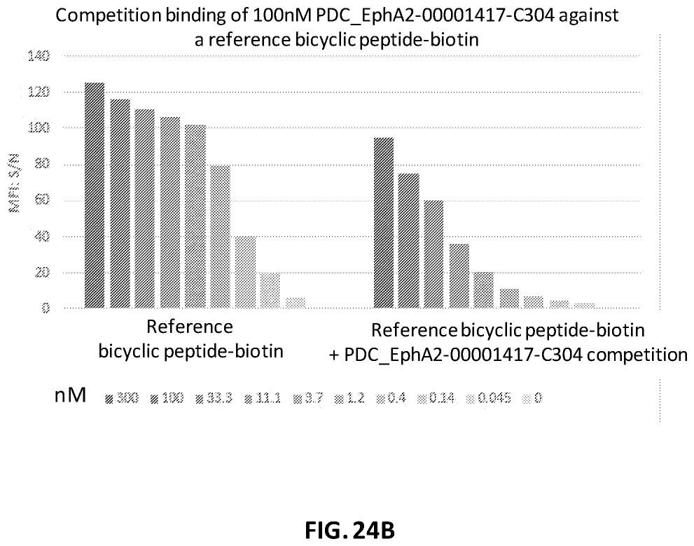

Competition cell binding for PDC_EphA2-00007196-C302, PDC_EphA2-00019440-C302, and PDC_EphA2-00019443-C302 tested against 50 nM of EphA2-Biotin-88 in HCT116 cells, see FIG. 24A.

Competition cell binding for PDC_EphA2-00001417-C304 with the biotinylated form of a reference bicyclic peptide in H1299 cells is illustrated in FIG. 24B.

### Example C6. Protocol-FACS Analysis for Compound Cell Internalization

Cells were dissociated with Accutase and stained with Zombie Violet in PBS (1:1000) at RT in the dark for 15 min. Cells were washed 3 times of cold PBS and resuspended at 2×10⁶ cells/mL in FACS buffer and 90 μL of cells were seeded into each well of a 96 wells plate. 10 μL of serial diluted compounds were added to 90 μL of cells in each well and incubated on ice for 1 hour. For each compound concentration, 4 groups of samples were prepared as following: 4 °C—No Quench; 4 °C-Quench; 37 °C—No Quench; and 37 °C-Quench. Cells were washed 3 times with cold PBS (@1,500 RPM for 3 min). Cells were then incubated with Streptavidin-AF488 (1:1000 dilution in FACS buffer) on ice for 30 min. Cells were washed 3 times with cold PBS (@1,500 RPM for 3 min), resuspended in 100 μL PBS and incubated at 4 °C or 37 °C for 1-3 hours. Cells were washed 3 times with cold PBS (@1,500 RPM for 3 min). No quench groups were incubated in 50 μL of cold FACS buffer and quench groups were incubated with 50 μL of 30 μg/ml of anti-Alexa488 in FACS buffer at 4 °C for 30 min. Cells were washed two times, resuspended in 100 μL of cold PBS and proceeded to signal acquisition on CytoFlex (Beckman Coulter Life Sciences, Indianapolis, IN).

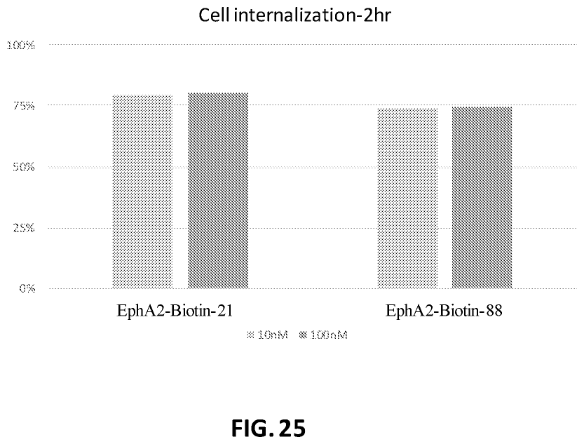

Internalization rate of biotinylated compound: EphA2-Biotin-21 and EphA2-Biotin-88 were measured in PC3 cells at 10 nM and 100 nM at 2 hour time point (see FIG. 25).

### Example C7. Surface Plasmon Resonance (SPR)

For analysis of peptide binding, a Biacore 8K instrument was used utilizing a SA chip. Biotin-Avi-His-EphA2 (SinoBiologicals, 13926-H27H-B) was immobilized on the chip using streptavidin-biotin chemistry at 25 °C in HBS-P+ buffer (10 mM HEPES, 150 mM NaCl, 0.05% Tween-20, pH 7.4) to a level of 1000-2000 RU (dependent on the analyte molecular weight). In case where the peptide had a biotin, Fc-EphA2 (Creative Biomarts, custom ordered) was immobilized on the chip using NHS EDC coupling chemistry at 25 °C in HBS-P+ buffer (10 mM HEPES, 150 mM NaCl, 0.05% Tween-20, pH 7.4) to a level of 1000-2000 RU (dependent on the analyte molecular weight). A dilution series of peptides was prepared in this buffer with a final DMSO concentration of 0.1% with a top peptide concentration between 10-100 nM and 9 further 2-fold dilutions. The SPR analysis was run at 25 °C at a flow rate of 30 μL/min in HBS-P+ with 0.1% DMSO running buffer and with a 120 second association and 1,200 second dissociation using single cycle kinetics methodology. All data was analyzed using Biacore Insight Evaluation Software version 4. Data was fitted using a Langmuir 1:1 binding model.

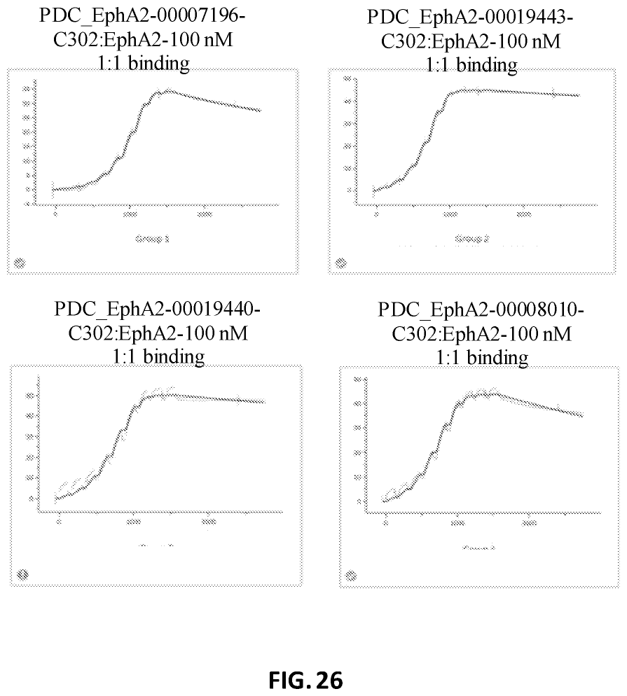

The results of the SPR study for PDC_EphA2-00007196-C302, PDC_EphA2-00019443-C302, PDC_EphA2-00019440-C302, and PDC_EphA2-00008010-C302 are illustrated in FIG. 26 and the Table C2 below.

TABLE C2
SPR Peptide Binding

| Compound | kₐ (M⁻¹s⁻¹) | k_{d} (s⁻¹) | K_{D} (M⁻¹) |
|---|---|---|---|
| PDC_EphA2- | 5.10e+5 | 2.06e−4 | 3.74e−10 |
| 00007196-C302 | | | |
| PDC_EphA2- | 1.65e+6 | 4.46e−5 | 2.70e−11 |
| 00019443-C302 | | | |
| PDC_EphA2- | 7.60e+5 | 6.21e−5 | 8.18e−11 |
| 00019440-C302 | | | |
| PDC_EphA2- | 9.19e+5 | 1.94e−4 | 2.11e−10 |
| 00008010-C302 | | | |

### Example C8. Biodistribution Study

Male athymic nude mice (Crown Bioscience) are inoculated with PC3 prostate cancer cells on their right shoulders. Animals (n=20) with tumor volume 200-390 mm³ are enrolled in the imaging study and categorized into three groups (n=4/group). Per group allocation, each subject is dosed intravenously (IV) with one 177Lu-radiolabeled peptide (100-120 μL, ~1000 μCi) and imaged using longitudinal multi-animal whole body single-photon emission computed tomography/computed tomography (SPECT/CT). Following the final imaging time point at 48 h post-radiotracer administration, all mice are euthanized, and tissues resected for ex vivo radioanalysis using gamma counter.

The examples and embodiments described herein are for illustrative purposes only and various modifications or changes suggested to persons skilled in the art are to be included within the spirit and purview of this application and scope of the appended claims.

What is claimed is:

1. A radiopharmaceutical conjugate comprising:
(a) a cyclic peptide that has avidity for ephrin type-A receptor 2 (EphA2), wherein the peptide comprises an amino acid sequence of Formula (I), or a pharmaceutically acceptable salt thereof,
X1-X2-X3-X4-X5-X6-X7-X8-X9-X10-X11-X12 Formula (I)
wherein,
X1 is an amino acid;
X2 is an amino acid comprising an aromatic ring, an N-methylated amino acid thereof, or a variant thereof;
X3 is a hydrophilic amino acid (e.g. N, Q, Cit, K or a variant thereof), glycine (G), Alanine (A) or a variant thereof (e.g., da, 2-Aminoisobutyric acid (Aib));
X4 is a hydrophobic amino acid (e.g., leucine (L)), a hydrophilic amino acid (e.g., citrulline (Cit)), or a variant thereof;
X5 is a hydrophilic amino acid, or a variant thereof;
X6 is a hydrophilic amino acid, an amino acid comprising an aromatic ring, or an N-methylated amino acid thereof;
X7 is an amino acid comprising an aromatic ring (e.g., W, F, or a variant thereof);
X8 is a hydrophobic amino acid, a hydrophilic amino acid, an N-methylated amino acid, or a variant thereof;
X9 is an amino acid comprising an aromatic ring (e.g., W or a variant thereof);
X10 is absent or a hydrophilic amino acid (e.g., Threonine (T) or a variant thereof);
X11 is absent or a hydrophilic amino acid; and
X12 is cysteine (C) or a variant thereof; and
(b)(i) a metal chelator configured to bind with a radionuclide, wherein the metal chelator is conjugated to the peptide, or (ii) a radionuclide covalently bound to the cyclic peptide.

2. A radiopharmaceutical conjugate comprising:
(a) a cyclic peptide that has avidity for ephrin type-A receptor 2 (EphA2), wherein the peptide comprises an amino acid sequence including deletion, substitution, and/or addition of one or several (e.g., 1-6) amino acids in the amino acid sequence of SEQ ID NO:1:
(SEQ ID NO: 1)
da-MeF-N-L-Hgl-MeF-W1Me-V-W1Me-T-E-C
or a pharmaceutically acceptable salt thereof,
wherein the cyclic peptide consists of 10 or 12 amino acid residues; and
(b)(i) a metal chelator configured to bind with a radionuclide, wherein the metal chelator is conjugated to the peptide, or (ii) a radionuclide covalently bound to the cyclic peptide.

3. The radiopharmaceutical conjugate of claim 1 or 2, comprising a metal chelator configured to bind with a radionuclide, wherein the metal chelator is conjugated to the peptide.

4. The radiopharmaceutical conjugate of claim 2, wherein 1-5 amino acids selected from the group consisting of 3^{rd} N, 4^{th} L, 6^{th} MeF, 10^{th} T and 11^{th} E of SEQ ID NO: 1 is/are deleted, optionally without additional addition and/or substitution.

5. The radiopharmaceutical conjugate of claim 2 or 4, wherein one to several (e.g., 1, 2, 3, 4 or 5) amino acids are added.

6. The radiopharmaceutical conjugate of any one of claim 2, 4, or 5, wherein one or more amino acid residues selected from the 2nd MeF, 6th MeF, 8th V and 11th E are substituted.

7. The radiopharmaceutical conjugate of any one of claim 1 or 4 to 6, wherein 1-2 amino acids selected from the group consisting of 10^{th} T and 11^{th} E of SEQ ID NO: 1 is/are deleted, optionally without additional addition and/or substitution.

8. The radiopharmaceutical conjugate of any one of claim 2 or 4 to 6, wherein the 8^{th} V is substituted.

9. The radiopharmaceutical conjugate of any one of claim 2 or 4 to 6, wherein the 11^{th} E is substituted.

10. The radiopharmaceutical conjugate of any one of claims 1 to 9, wherein the metal chelator is conjugated to the N-terminus of the peptide.

11. The radiopharmaceutical conjugate of any one of claims 1 to 10, further comprising a radionuclide bound to the metal chelator.

12. The radiopharmaceutical conjugate of claim 11, wherein the radionuclide is an alpha particle-emitting radionuclide.

13. The radiopharmaceutical conjugate of claim 12, wherein the alpha particle-emitting radionuclide is selected from Ac-225, Bi-213, Bi-209, Tb-149, Ra-223, Th-227, Fr-223, Gd-148, Th-229, Pb-212, and Po-213.

14. The radiopharmaceutical conjugate of claim 12, wherein the alpha particle-emitting radionuclide is Ac-225.

15. The radiopharmaceutical conjugate of claim 11, wherein the radionuclide is a beta particle-emitting radionuclide (e.g., Cu-67, Lu-177, Y-90, Rh-105, Yb-175, Tm-167, Pm-153, Sm-153, or In-111).

16. The radiopharmaceutical conjugate of claim 15, wherein the beta particle-emitting radionuclide is Lu-177.

17. The radiopharmaceutical conjugate of claim 11, wherein the radionuclide is a positron-emitting radionuclide (e.g., Ga-68, Cu-62, Cu-64, Zr-89, or Tb-152).

18. The radiopharmaceutical conjugate of claim 17, wherein the positron-emitting radionuclide is Ga-68 or Cu-64.

19. The radiopharmaceutical conjugate of any one of claims 1 to 18, wherein the metal chelator comprises DOTA, DOTA-GA, pBn-DOTA, pBn-SCN-DOTA, NH2-DOTA, NH2-DOTA-GA, p-NCS-Bn-DOTA-GA, p-NH2-Bn-oxo-DO3A, p-SCN-Bn-oxo-DO3A, NOTA, NODA-GA, NH2-NODA-GA, p-NCS-Bn-NODA-GA, p-NH2-Bn-NOTA, p-SCN-Bn-NOTA, NCS-MP-NODA, NH2-MPAA-NODA, PCTA, p-NH2-Bn-PCTA, p-SCN-Bn-PCTA, p-SCN-Bn-HEHA, H2-MACROPA-NCS, H1-MACROPA, H2-MACROPA-NH2, H4-OCTAPA, tetra-(S, S, S, S)-Me-DOTA, tetra-(S, S, S, S)-Et-DOTA, tetra-(S, S, S, S)-iBu-DOTA, or maleimide-nBu-DOTA.

20. The radiopharmaceutical conjugate of claim 19, wherein the metal chelator has a structure of

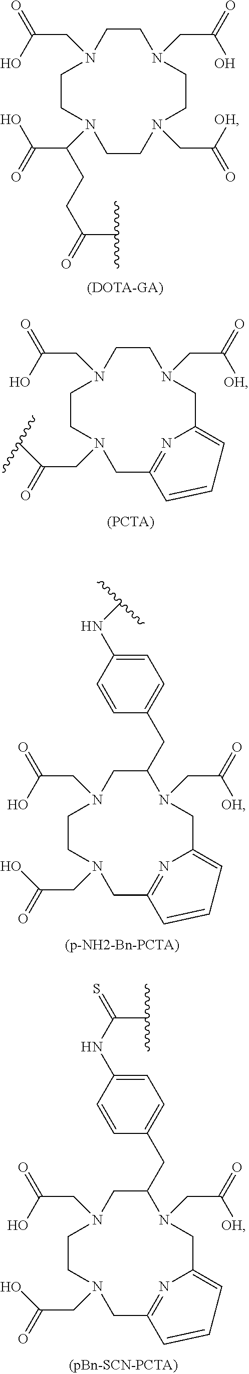 [SMILES: *C(=O)CCC(C(=O)O)N1CCN(CC(=O)O)CCN(CC(=O)O)CCN(CC(=O)O)CC1 ; *C(=O)CN1CCN(CC(=O)O)CCN(CC(=O)O)Cc2cccc(n2)C1 ; *Nc1ccc(CC2CN(CC(=O)O)CCN(CC(=O)O)Cc3cccc(n3)CN2CC(=O)O)cc1 ; *C(=S)Nc1ccc(CC2CN(CC(=O)O)CCN(CC(=O)O)Cc3cccc(n3)CN2CC(=O)O)cc1]

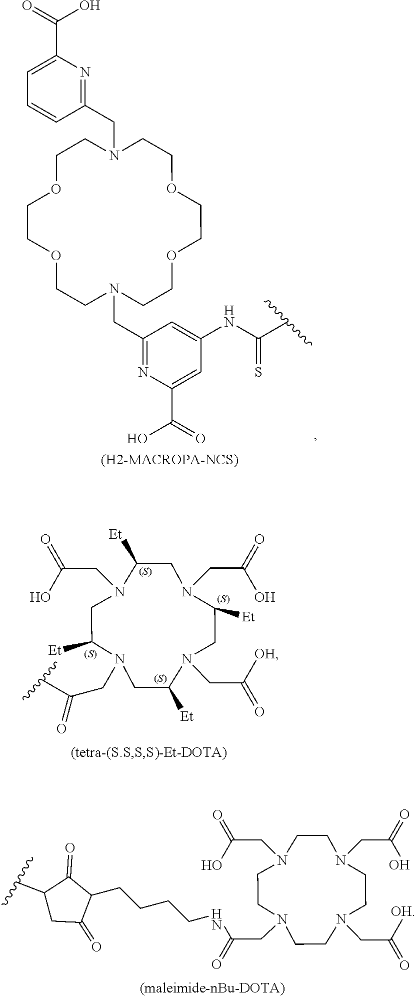

21. The radiopharmaceutical conjugate of claim 19, wherein the metal chelator has a structure of

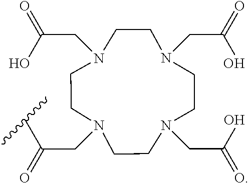 [SMILES: *C(=O)CN1CCN(CC(=O)O)CCN(CC(=O)O)CCN(CC(=O)O)CC1]

22. The radiopharmaceutical conjugate of any one of claims 1 to 21, further comprising a linker that covalently connects the peptide with the metal chelator.

23. The radiopharmaceutical conjugate of claim 22, wherein the conjugate has a structure of: 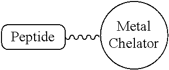
wherein [symbol]
represents the linker.

24. The radiopharmaceutical conjugate of claim 22 or 23, wherein the linker is attached to the peptide via a non-terminal amino acid residue of the peptide.

25. The radiopharmaceutical conjugate of claim 24, wherein the linker is attached to the 5^{th} amino acid residue or X5.

26. The radiopharmaceutical conjugate of claim 24, wherein the linker is attached to the 8^{th} amino acid residue or X8.

27. The radiopharmaceutical conjugate of claim 24, wherein the linker is attached to the 11^{th} amino acid residue or X11.

28. The radiopharmaceutical conjugate of any one of claims 24 to 27, wherein linker is attached to a lysine of the peptide.

29. The radiopharmaceutical conjugate of claim 22 or 23, wherein the linker is attached to the peptide via the N terminus of the peptide.

30. The radiopharmaceutical conjugate of claim 22 or 23, wherein the linker is attached to the peptide via the C terminus of the peptide.

31. The radiopharmaceutical conjugate of any one of claims 22 to 30, wherein the linker is a bond.

32. The radiopharmaceutical conjugate of any one of claims 22 to 30, wherein the linker comprises 3 to 30 intervening atoms between the metal chelator and the peptide.

33. The radiopharmaceutical conjugate of any one of claims 22 to 30, wherein the linker comprises 6 to 18 intervening atoms between the metal chelator and the peptide.

34. The radiopharmaceutical conjugate of claim 32 or 33, wherein the intervening atoms comprise 1 to 6 nitrogen and 0 to 4 oxygen.

35. The radiopharmaceutical conjugate of any one of claims 22 to 30 or 32 to 34, wherein the linker comprises one or more amino acid residues.

36. The radiopharmaceutical conjugate of claim 35, wherein the linker comprises an amino acid residue selected from a lysine residue, an alanine residue, a glycine residue, a d-phenylalanine and a phenylalanine residue.

37. The radiopharmaceutical conjugate of any one of claims 22 to 30 or 32 to 36, wherein the linker comprises one or more structures selected from AEEA, AEEP, AEEEP, and AEEEEP.

38. The radiopharmaceutical conjugate of any one of claims 22 to 30, wherein the linker has a structure of Formula (II-1) 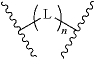
wherein each L is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, —(C₁-C₃₀ alkylene)-O—, —O—(C₁-C₃₀ alkylene)-, —(C₁-C₃₀ alkylene)-NR^{L}—, —NR^{L}—(C₁-C₃₀ alkylene)-, —(C₁-C₃₀ alkylene)-N(R^{L})₂—, or —N(R^{L})₂—(C₁-C₃₀ alkylene)-; and
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
n is 1 to 20.

39. The radiopharmaceutical conjugate of claim 38, wherein the linker comprises a structure of Formula (II-1a), 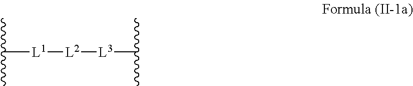
wherein each of L¹ and L³ is independently —O—, —NR^{L}—, —N(R^{L})₂—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—; and
L² is absent, substituted or unsubstituted C₁-C₃₀ alkylene, or substituted or unsubstituted C₁-C₃₀ heteroalkylene.

40. The radiopharmaceutical conjugate of claim 39, wherein L¹ is —NH—.

41. The radiopharmaceutical conjugate of claim 39 or 40, wherein L² is substituted or unsubstituted C₁-C₃₀ alkylene, or substituted or unsubstituted C₁-C₃₀ heteroalkylene.

42. The radiopharmaceutical conjugate of claim 39 or 40, wherein L² is substituted or unsubstituted C₁-C₁₈ alkylene, or substituted or unsubstituted C₁-C₁₈ heteroalkylene.

43. The radiopharmaceutical conjugate of any one of claims 39 to 42, wherein L² is optionally substituted with one or more substituents selected from —OH, —SH, oxo, amino, C₁-C₆ alkyl, C₁-C₆ hydroxyalkyl, C₁-C₆ haloalkyl, C₁-C₆ aminoalkyl, —C(═O)OR^{L}, —OC(═O)R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}; and the C₁-C₆ alkyl is further optionally substituted with one or more substituents chosen from —OH, —SH, oxo, amino, C₆-C₁₀ aryl, 6- to 10-membered heteroaryl, —C(═O)OR^{L}, —OC(═O)R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}.

44. The radiopharmaceutical conjugate of any one of claims 39 to 43, wherein L³ is —NH—.

45. The radiopharmaceutical conjugate of claim 39, wherein the linker has a structure of

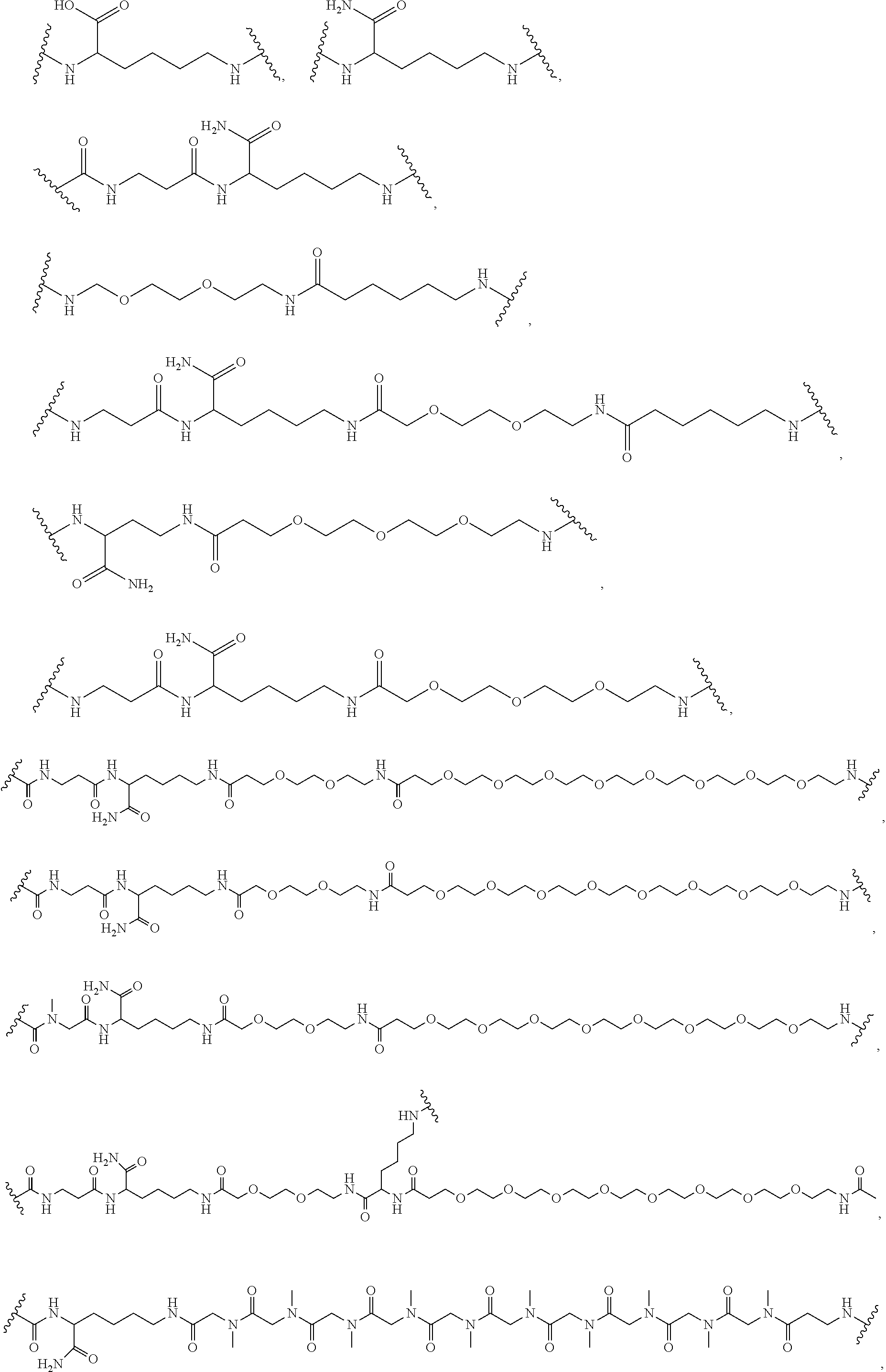

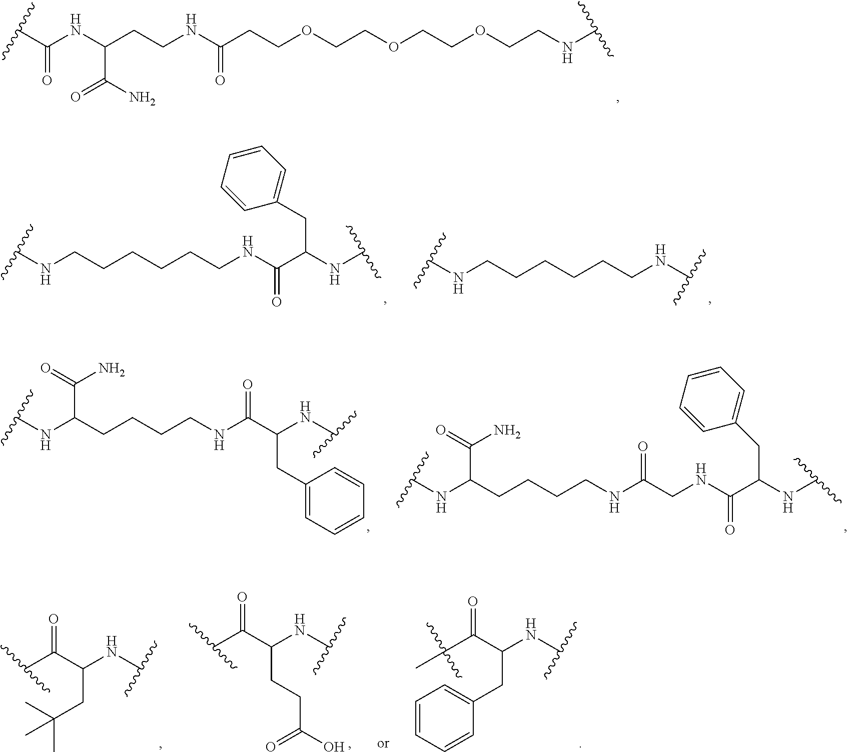

46. The radiopharmaceutical conjugate of claim 39, wherein the linker has a structure of

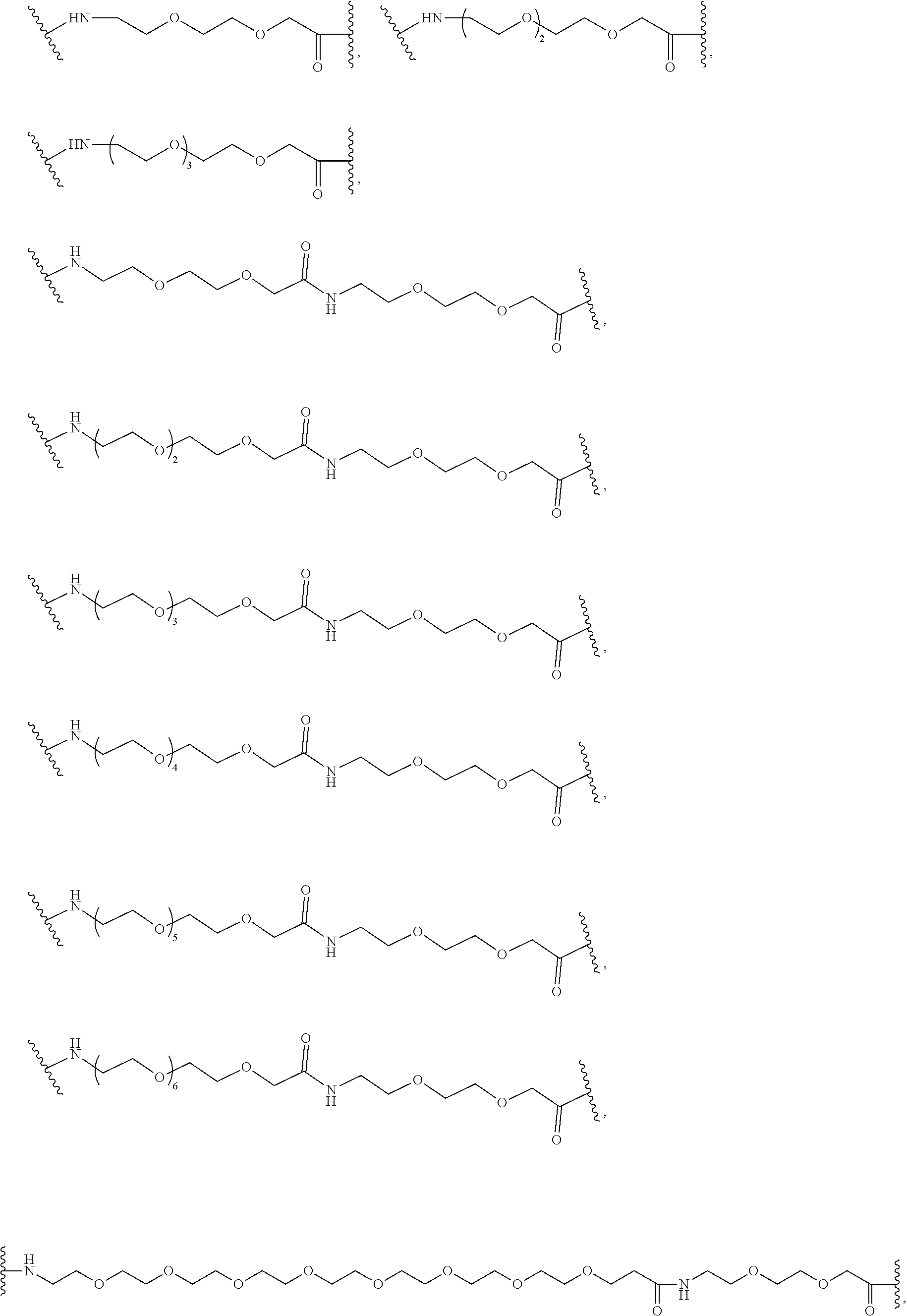

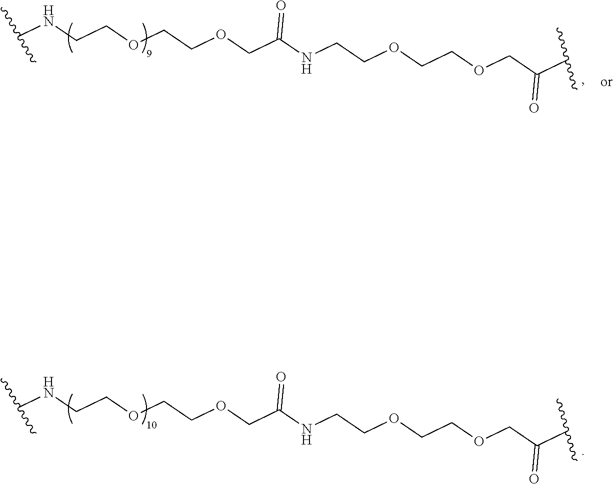

47. The radiopharmaceutical conjugate of any one of claims 1 to 46, wherein the peptide or the pharmaceutically accepted salt thereof has a cyclic structure, wherein the first amino acid (or X1) is covalently linked to the last amino acid (or X12).

48. The radiopharmaceutical conjugate of any one of claims 1 to 46, wherein the peptide or the pharmaceutically accepted salt thereof has a cyclic structure having an amino acid in the first residue X1 and a cysteine residue or a variant thereof, and wherein the amino acid in X1 and the cysteine residue or a variant thereof form a covalent bond.

49. The radiopharmaceutical conjugate of claim 48, wherein the peptide consists of an amino acid sequence selected from SEQ ID NOs: 1-122, 159-163, and 165-171, and the peptide has a cyclic structure having a cysteine residue or a variant thereof at 12th residue, and wherein the amino acid X1 and the cysteine residue or a variant thereof at 12th residue are connected via a covalent bond (e.g., by reacting a chloroacetyl group in the amino acid of X1 with the cysteine residue or a variant thereof).

50. The radiopharmaceutical conjugate of claim 48, wherein the peptide consists of an amino acid sequence selected from SEQ ID NOs: 123-149 and 164, and the peptide has a cyclic structure having a cysteine residue or a variant thereof at 10th residue, and wherein the amino acid X1 and the cysteine residue or a variant thereof at 10th residue are connected via a covalent bond.

51. The radiopharmaceutical conjugate of any one of claim 1 or 10 to 50, wherein X3 is a hydrophilic amino acid.

52. The radiopharmaceutical conjugate of claim 51, wherein X3 is an amino acid comprising an electrically charged side chain (e.g., K or a variant thereof), an amino acid comprising a polar uncharged side chain (e.g., Q, Cit, N, or a variant thereof), G, A, or variant thereof.

53. The radiopharmaceutical conjugate of any one of claim 1 or 10 to 52, wherein X4 is a hydrophobic amino acid.

54. The radiopharmaceutical conjugate of claim 53, wherein X4 is an amino acid comprising a hydrophobic side chain (e.g., L), an amino acid comprising a polar uncharged side chain (e.g., Cit or a variant thereof).

55. The radiopharmaceutical conjugate of any one of claim 1 or 10 to 54, wherein X5 is a hydrophilic amino acid.

56. The radiopharmaceutical conjugate of claim 55, wherein X5 is an amino acid comprising an electrically charged side chain (e.g., E, Hgl, D, or a variant thereof), or an amino acid comprising a polar uncharged side chain (e.g., Q, Cit, Hgn, N, or a variant thereof).

57. The radiopharmaceutical conjugate of any one of claim 1 or 10 to 56, wherein X6 is a hydrophilic amino acid.

58. The radiopharmaceutical conjugate of claim 57, wherein X6 is an amino acid comprising an electrically charged side chain (e.g., E, Hgl, D, or a variant thereof), or an amino acid comprising a polar uncharged side chain (e.g., Q, Cit, Hgn, N, or variant).

59. The radiopharmaceutical conjugate of any one of claim 1 or 10 to 58, wherein X11 is a hydrophilic amino acid.

60. The radiopharmaceutical conjugate of claim 59, wherein X11 is an amino acid comprising an electrically charged side chain (e.g., E, Hgl, D, R, hArg, K or a variant thereof), or an amino acid comprising a polar uncharged side chain (e.g., Q, Cit, Hgn, N, or a variant thereof).

61. The radiopharmaceutical conjugate of claim 59, wherein X11 is arginine (R), asparagine (N), aspartate (D), glutamine (Q), lysine (K), or an unnatural hydrophilic amino acid.

62. The radiopharmaceutical conjugate of any one of claims 1 to 61, wherein the peptide has an amino acid sequence of Formula (I), or a pharmaceutically acceptable salt thereof,
X1-X2-X3-X4-X5-X6-X7-X8-X9-X10-X11-X12 Formula (I)
wherein,
X1 is an amino acid;
X2 is F, or a variant thereof that replaces the unsubstituted phenyl ring of F with
(i) a phenyl ring substituted by 1 or 2 substituents each independently selected from —OH, —CN, and —C₁₋₃ alkyl, or
(ii) a 6-membered heteroaryl ring optionally substituted by 1 or 2 substituents each independently selected from —OH, —CN, and —C₁₋₃ alkyl,
wherein the F or the variant thereof is optionally N-methylated;
X3 is a hydrophilic amino acid (e.g. N, Q, Cit, K or a variant thereof), G, Aib, Hgn, Ala, or a variant thereof (e.g., da);
X4 is a hydrophobic amino acid (e.g., an amino acid having 4 or more carbon atoms in a side chain comprising a linear, branched, or cyclic carbon chain), and wherein X4 is optionally N-methylated (e.g., Cit or a variant thereof);
X5 is an amino acid (e.g., a hydrophilic amino acid; Dab, Dap, R, E or a variant thereof; or an amino acid with a functional side chain);
X6 is an N-methylated amino acid thereof;
X7 is a W, Y, or a variant thereof (e.g., an amino acid having either a 6-membered aryl or heteroaryl, or a 9- or 10-membered bi-cyclic aryl or heteroaryl linked to the alpha-carbon through a carbon (e.g., a methylene group), wherein the 6-, 9-, and 10-membered heteroaryl has one heteroatom (e.g., N), and wherein the 6-, 9-, and 10-membered aryl or heteroaryl is optionally substituted by 1 or 2 substituents independently selected from —CH₃, -ethyl, —Cl, and —F);
X8 is an amino acid with —H on the alpha-amino group;
X9 is W or Y or a variant thereof; (e.g., W or a variant thereof);
X10 is absent, or a polar amino acid (e.g., T or a variant thereof);
X11 is absent, or an amino acid (e.g., a hydrophilic amino acid; Dab, Dap, R, E or a variant thereof; or an amino acid with a functional side chain); and
X12 is C or a variant thereof.

63. The radiopharmaceutical conjugate of claim 62, wherein X8 is KCOpipzaa, N, Cit, Qglucamine, hCit, K, KAc, Aib, Alb, DapAc, OrnAc, A, T, alT, Norleucine, Norvaline, Hgl, E, Hgn, Q, I, or L.

64. The radiopharmaceutical conjugate of claim 62 or 63, wherein X11 is absent, arginine (R), asparagine (N), aspartate (D), glutamine (Q), lysine (K), or an unnatural hydrophilic amino acid.

65. The radiopharmaceutical conjugate of any one of claims 1 to 61, wherein the peptide has an amino acid sequence of Formula (Ia), or a pharmaceutically acceptable salt thereof,
X1-X2-X3-X4-X5-X6-X7-X8-X9-X12 Formula (Ia)
wherein,
X1 is an amino acid (e.g., D-amino acid);
X2 is an amino acid comprising an aromatic ring, an N-methylated amino acid thereof, or a variant thereof;
X3 is a hydrophilic amino acid (e.g., N, Q, Cit, K or a variant thereof), G, A, or a variant thereof (e.g., da, Aib);
X4 is a hydrophobic amino acid, or a hydrophilic amino acid (e.g., Cit or a variant thereof);
X5 is a hydrophilic amino acid (e.g., Dab, Dap, R, E, Q, D, K), or a variant thereof;
X6 is a hydrophilic amino acid, an amino acid comprising an aromatic ring (e.g., W, or F, or a variant thereof), or an N-methylated amino acid thereof;
X7 is an amino acid comprising an aromatic ring (e.g., W, F, or a variant thereof);
X8 is a hydrophobic amino acid, a hydrophilic amino acid, or an N-methylated amino acid;
X9 is an amino acid comprising an aromatic ring (e.g., W, F or a variant thereof); and
X12 is C or a variant thereof.

66. The radiopharmaceutical conjugate of any one of claims 1 to 61, wherein the peptide has an amino acid sequence according to Formula (I), or a pharmaceutically acceptable salt thereof,
X1-X2-X3-X4-X5-X6-X7-X8-X9-X10-X11-X12 Formula (I)
wherein,
X1 is an amino acid (e.g., D-amino acid);
X2 is an amino acid comprising an aromatic ring, an N-methylated amino acid thereof, or a variant thereof;
X3 is a hydrophilic amino acid (e.g., N, Q, Cit, K or a variant thereof), G, A, or a variant thereof (e.g., da, Aib);
X4 is a hydrophobic amino acid, or a hydrophilic amino acid (e.g., Cit or a variant thereof);
X5 is a hydrophilic amino acid (e.g., Dab, Dap, R, E, Q, D, K), or a variant thereof;
X6 is a hydrophilic amino acid, an amino acid comprising an aromatic ring (e.g., W, or F, or a variant thereof), or an N-methylated amino acid thereof;
X7 is an amino acid comprising an aromatic ring (e.g., W, F, or a variant thereof);
X8 is a hydrophobic amino acid, a hydrophilic amino acid, or an N-methylated amino acid;
X9 is an amino acid comprising an aromatic ring (e.g., W, F or a variant thereof);
X10 is a hydrophilic amino acid (e.g., T, S, N, Q, K, Cit, or a variant thereof);
X11 is a hydrophilic amino acid; and
X12 is C or a variant thereof.

67. The radiopharmaceutical conjugate of claim 66, wherein X8 is KCOpipzaa, N, Cit, Qglucamine, hCit, K, KAc, Aib, Alb, DapAc, OrnAc, A, T, alT, Norleucine, Norvaline, Hgl, E, Hgn, Q, I, or L.

68. The radiopharmaceutical conjugate of claim 66 or 67, wherein X11 is arginine (R), asparagine (N), aspartate (D), glutamine (Q), lysine (K), or an unnatural hydrophilic amino acid.

69. The radiopharmaceutical conjugate of any one of claims 1 to 61, wherein
X1 is an amino acid (e.g., D-amino acid);
X2 is F, Y, W, a variant thereof (e.g., Hgn), or an N-methylated amino acid thereof;
X3 is N, Q, Cit, G, Aib, K, A, or a variant thereof;
X4 is G, A, Cit, L, or a variant thereof (e.g., G substituted with straight or branched C₁₋₅ alkyl, G substituted with C₃₋₇ cycloalkyl, or A substituted with C₃₋₇ cycloalkyl);
X5 is a hydrophilic L-amino acid, wherein the L-amino acid comprises a functional group selected from —NH₂, —C(O)OH, —NHC(NH)NH₂, —NHC(O)NH₂, —C(O)NH₂, and —NHC(O)CH₃;
X6 is a hydrophilic amino acid, F, Y, W, N-methylated amino acid thereof, or a variant thereof, wherein the hydrophilic amino acid comprises a functional group selected from —C(O)OH, —C(O)NH₂, and —NHC(O)CH₃;
X7 is F, W, or a variant thereof;
X8 is G substituted with one or two straight or branched C₁₋₅ alkyl, G substituted with C₃₋₇ cycloalkyl, A substituted with C₃₋₇ cycloalkyl, or a hydrophilic L-amino acid wherein the hydrophilic L-amino acid comprises-NH₂, one or more —OH, —C(O)OH, —NHC(NH)NH₂, —NHC(O)NH₂, —C(O)NH₂, or —NHC(O)CH₃; or the hydrophilic amino acid comprises a zwitterion;
X9 is F, W, or a variant thereof;
X10 is absent, Q, S, K, Cit, N, T, or a variant thereof (e.g., Q, S, K, Cit, N, or T optionally substituted with straight or branched C₁₋₅ alkyl) or an L-amino acid comprising-NHC(NH)NH₂, —NHC(O)NH₂, —C(O)NH₂, or —NHC(O)CH₃;
X11 is absent, E, Q, R, Cit, K, D, or N, or a variant thereof; and
X12 is C or a variant thereof.

70. The radiopharmaceutical conjugate of claim 69, wherein X8 is KCOpipzaa, N, Cit, Qglucamine, hCit, K, KAc, Aib, Alb, DapAc, OrnAc, A, T, alT, Norleucine, Norvaline, Hgl, E, Hgn, Q, I, or L.

71. The radiopharmaceutical conjugate of claim 69 or 70, wherein X11 is absent, arginine (R), asparagine (N), aspartate (D), glutamine (Q), lysine (K), or an unnatural hydrophilic amino acid.

72. The radiopharmaceutical conjugate of any one of claim 1 or 10 to 71, wherein a variant of an amino acid is selected from amino acids having one, two or three substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, —S(═O)₂NH₂, —S(═O)₂NH(C₁-C₃alkyl), —S(═O)₂N(C₁-C₃alkyl)₂, C₁-C₆ alkyl, C₁-C₆ heteroalkyl, C₁-C₆ alkoxy, C₆-C₁₀ aryl, C₃-C₆ cycloalkyl, 6-10 membered heterocycloalkyl, and 6-10 membered heteroaryl.

73. The radiopharmaceutical conjugate of claim 72, wherein the variant is selected from amino acids having one or two substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂-C₁-C₃alkyl, —C(═O)NH₂, —C(═O)NH(C₁-C₃alkyl), —C(═O)N(C₁-C₃alkyl)₂, and C₁-C₆ alkyl.

74. The radiopharmaceutical conjugate of any one of claim 1 or 10 to 71, wherein a variant of an amino acid is selected from amino acids that have the similar hydrophilicity or hydrophobicity compared to the amino acid.

75. The radiopharmaceutical conjugate of any one of claim 1 or 10 to 71, wherein a variant of an amino acid is selected from amino acids that have the same functional group as the amino acid, and wherein the variant has a different length of a side chain compared to the amino acid.

76. The radiopharmaceutical conjugate of any one of claim 1 or 10 to 75, wherein the variant has a molecular weight that does not vary for more than 14, 28, 30, 45 or 60 g/mol compared to the amino acid.

77. The radiopharmaceutical conjugate of any one of claims 1 to 71, wherein the peptide has an amino acid sequence according to Formula (I), or a pharmaceutically acceptable salt thereof,
X1-X2-X3-X4-X5-X6-X7-X8-X9-X10-X11-X12 Formula (I)
wherein,
X1 is da, df3CON, dkCOpipzaa, dahp, dDab-NH₂-Ph3-SO₂F, dDap-NH₂-Ph3-SO₂F, dDap-NH₂-Ph4-SO₂F, dCit, Aib, G, Norvaline, Norleucine, d4PyCON, or dhAla;
X2 is MeF, Me3Py, MeF3CON, MeF3F, Me4Py, or MeY (Me);
X3 is absent, N, Q, Cit, G, Aib, Hgn, hCit, norCit, LysAc, OrnAc, Ala, or da;
X4 is L, Cbg, Chg, Cba, Cha, Ahx, Dahp, Cit, I, V, Norleucine, or Norvaline;
X5 is Hgl, Hgn, Dab, Dap, DabAc, DapAc, R, hArg, E, or D;
X6 is absent, MeF, MeE, Me3Py, Me4Py, MeF4F, MeF4F, MeF4C, or MeY;
X7 is W1Me, W1Me7Cl, W1Me7N, W, F, 7-AzaTrp, W7Me, W1Et, W1Me7Br, W1Me7OMe, or W1Me6O7Cl;
X8 is V, KCOpipzaa, N, Cit, Qglucamine, hCit, K, KAc, Aib, Alb, DapAc, OrnAc, A, T, alT, Norleucine, Norvaline, Hgl, E, Hgn, Q, I, or L;
X9 is W1Me, W1Me7Cl, W1Me7N, F23dMe, W1Et, W7Me, W, F, or 7-AzaTrp;
X10 is absent, T, Q, S, Hgn, Alpha-methylserine, hSer, hThr, N, OrnAc, LysAc, Cit, or hCit;
X11 is absent, E, Hgn, R, hArg, Cit, hCit, Hgl, Orn, D, N, Q, DapAc, OrnAc, DabAc, or norCit; and
X12 is C, hCys, CdMe, C3RMe, C3SMe, Selenocysteine, dc, or Penicillamine.

78. The radiopharmaceutical conjugate of claim 77, wherein X8 is KCOpipzaa, N, Cit, Qglucamine, hCit, K, KAc, Aib, Alb, DapAc, OrnAc, A, T, alT, Norleucine, Norvaline, Hgl, E, Hgn, Q, I, or L.

79. The radiopharmaceutical conjugate of claim 77 or 78, wherein X11 is absent, Hgn, R, hArg, Cit, hCit, Hgl, Orn, D, N, Q, DapAc, OrnAc, DabAc, or norCit.

80. The radiopharmaceutical conjugate of any one of claims 62 to 79, wherein
X7 is W1Me or a variant thereof; and
X9 is W1Me or a variant thereof.

81. The radiopharmaceutical conjugate of any one of claims 62 to 80, wherein
X7 is W1Me, W1MeCl, W1MeBr, Nal1, Nal2, W1Et, 3Bzf, 3Bzt, F23dC, W1Me7N, or F23dMe;
X8 is V, KCOpipzaa, N, Cit, hCit, KAc, DapAc, OrnAc, A, T, alT, Aib, Alb, Qglucamine, Hgl, Q, E, Hgn, or K; and
X9 is W1Me, Nal1, W1Et, Nal21N, 3Bzf, 3Bzt, Nal18N, F23dMe, or F23dC.

82. The radiopharmaceutical conjugate of claim 81, wherein X8 is KCOpipzaa, N, Cit, hCit, KAc, DapAc, OrnAc, A, T, alT, Aib, Alb, Qglucamine, Hgl, Q, E, Hgn, or K.

83. A radiopharmaceutical conjugate comprising:
(a) a cyclic peptide that has avidity for ephrin type-A receptor 2 (EphA2), wherein the peptide consists of a sequence of Formula (I),
X1-X2-X3-X4-X5-X6-X7-X8-X9-X10-X11-X12 Formula (I)
or a pharmaceutically acceptable salt thereof,
wherein
each of X1, X2, X3, X4, X5, X6, and X8 is independently an amino acid;
X7 is W1Me or a variant thereof;
X9 is W1Me or a variant thereof;
each of X10 and X11 is independently absent or an amino acid; and
X12 is cysteine (C) or a variant thereof;
(b) a metal chelator configured to bind with a radionuclide; and
(c) optionally, a linker that connects the peptide with the metal chelator.

84. The radiopharmaceutical conjugate of claim 83, wherein X8 is KCOpipzaa, N, Cit, Qglucamine, hCit, K, KAc, Aib, Alb, DapAc, OrnAc, A, T, alT, Norleucine, Norvaline, Hgl, E, Hgn, Q, I, or L.

85. The radiopharmaceutical conjugate of claim 83 or 84, wherein X11 is absent, arginine (R), asparagine (N), aspartate (D), glutamine (Q), lysine (K), or an unnatural hydrophilic amino acid.

86. A radiopharmaceutical conjugate comprising:
(a) a cyclic peptide that has avidity for ephrin type-A receptor 2 (EphA2), wherein the peptide has an amino acid sequence of Formula (I),
X1-X2-X3-X4-X5-X6-X7-X8-X9-X10-X11-X12 Formula (I)
wherein,
X1 is any D- or L-amino acid;
X2 has a structure of 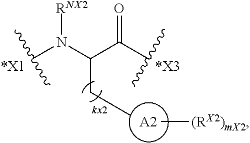
wherein
ring A2 is phenyl or a 6-membered heteroaryl (e.g., heteroaryl having 1 or 2 N);
R^{X2} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
kx2 is 0, 1, 2, or 3;
mx2 is 0, 1, 2, 3 or 4;
R^{NX2} is H, C₁-C₆alkyl, or C₁-C₆haloalkyl;
X1 indicates the point of attachment to X1; and,
X3 indicates the point of attachment to X3; 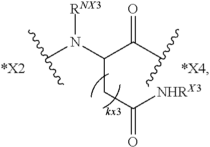
X3 has a structure of wherein
kx3 is 0, 1, 2, or 3;
R^{NX3} is H, C₁-C₆alkyl, or C₁-C₆haloalkyl;
R^{X3} is H, C₁-C₆alkyl, C₁-C₆haloalkyl, C₁-C₆hydroxyalkyl, C₁-C₆aminoalkyl, or C₁-C₆heteroalkyl;
X2 indicates the point of attachment to X2; and,
X4 indicates the point of attachment to X4;
X4 is a hydrophobic amino acid (e.g., amino acid having 4 or more carbon atoms in a side chain comprising a linear, branched, or cyclic carbon chain), and wherein X4 is optionally N-alkylated by a C₁₋₃ alkyl group;
X5 is a hydrophilic L-amino acid, such as an amino acid having a structure of 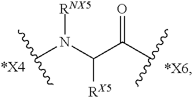
wherein:
R^{NX5} is H, —CN, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA},
R^{X5} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═NR^{b})NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl; wherein the alkyl, haloalkyl, hydroxyalkyl, aminoalkyl, or heteroalkyl is optionally and independently substituted with one or more R^{XA};
provided that at least one of R^{NX5} and R^{X5} comprises a moiety selected from —OH, —NH₂, and —NH— (e.g., —NH—C(═NH)—NH₂, —CO—NH₂, —NH₂, —COOH, —C(OH)—C₀₋₆ alkyl, —NH—CO—C₁₋₆ alkyl);
*X4 indicates the point of attachment to X4; and,
*X6 indicates the point of attachment to X6;
X6 is 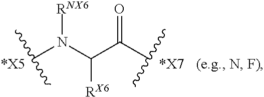
wherein
R^{NX6} is H, C₁-C₆alkyl, or C₁-C₆haloalkyl;
R^{X6} is —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(—NR^{b})NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl or heteroaryl; wherein the alkyl, haloalkyl, hydroxyalkyl, aminoalkyl, heteroalkyl, cycloalkyl, heterocycloalkyl, aryl or heteroaryl is optionally and independently substituted with one or more R^{XA},
*X5 indicates the point of attachment to X5; and,
*X7 indicates the point of attachment to X7;
X7 has a structure of 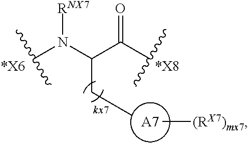
wherein
R^{NX7} is H, C₁-C₆alkyl, or C₁-C₆haloalkyl;
ring A7 is an aryl or heteroaryl;
R^{X7} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂-halogen, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
kx7 is 0, 1, 2, or 3;
mx7 is 0, 1, 2, 3, 4 or 5;
*X6 indicates the point of attachment to X6; and,
*X8 indicates the point of attachment to X8;
X8 is an L-amino acid comprising an —H on the alpha-amino group;
X9 has a structure of 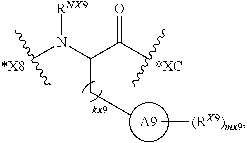
wherein
R^{NX9} is H, C₁-C₆alkyl, or C₁-C₆haloalkyl;
ring A9 is an aryl or heteroaryl;
R^{X9} is each independently halogen, —CN, —NO₂, —OH, —OR^{a}, —OC(═O)R^{a}, —OC(═O)OR^{b}, —OC(═O)NR^{c}R^{d}, —SH, SF₅, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein the alkyl, haloalkyl, hydroxyalkyl, aminoalkyl, heteroalkyl, alkenyl, alkynyl, cycloalkyl, or heterocycloalkyl is optionally and independently substituted with one or more R^{XA};
kx9 is 0, 1, 2, or 3;
mx9 is 0, 1, 2, 3, 4, or 5;
*X8 indicates the point of attachment to X8; and,
*XC indicates the point of attachment to (i) X10 or (i) when X10 and X11 are absent,
X12;
X10 is absent or an L-amino acid;
X11 is absent or an L-amino acid; provided that when X10 is absent, then X11 is also absent; and
X12 is an L-amino acid having a reactive thiol group, such as Cys and Cys variants;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R and R^{XA} is independently halogen, —CN, —OH, —OC₁-C₆alkyl, SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NR^{b}C(═NR^{b})NR^{c}R^{d}, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl;
(b) a metal chelator configured to bind with a radionuclide; and
(c) optionally, a linker that connects the peptide with the metal chelator.

87. The radiopharmaceutical conjugate of claim 86, wherein ring A7 is a 6-membered aryl or heteroaryl, or a 9- or 10-membered bicyclic aryl or heteroaryl, wherein the 6-, 9- or 10-membered heteroaryl has one heteroatom selected from N, O, and S.

88. The radiopharmaceutical conjugate of claim 86 or 87, wherein R^{NX7} is H.

89. The radiopharmaceutical conjugate of any one of claims 86 to 88, wherein each R^{X7} is independently selected from —CH₃, -ethyl, —Cl, and —F, and mx7 is 0, 1, or 2.

90. The radiopharmaceutical conjugate of claim 86, wherein X7 is W1Me, Nal1, Nal2, W1Et, Nal21N, 3Bzf, 3Bzt, Nal15N, Nal14N, Nal24N, Nal28N, F23dMe, F23dC, W1Me7N, or W1Me7Cl.

91. The radiopharmaceutical conjugate of claim 90, wherein X7 is W1Me, F23dMe or W1Me7Cl.

92. The radiopharmaceutical conjugate of any one of claims 86 to 91, wherein X9 is 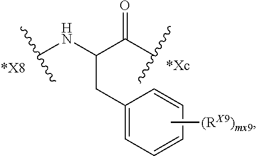
each R^{X9} is independently selected from —OH, CN, NH₂, C₁-C₃alkyl, —C1, —F, —Br, —CONH₂, and —SO₂F.

93. The radiopharmaceutical conjugate of any one of claims 86 to 92, wherein

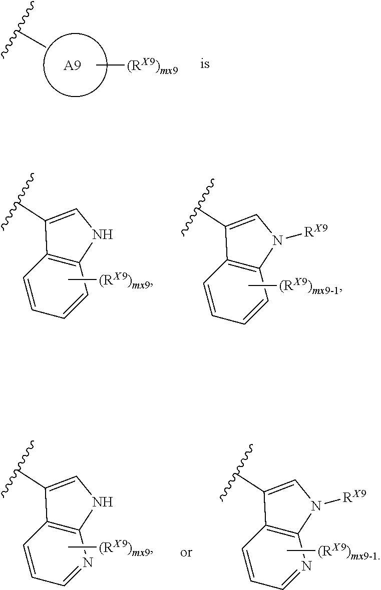

94. The radiopharmaceutical conjugate of any one of claims 86 to 93, wherein R^{X9} is each independently halogen, —CN, —NO₂, —OH, —OR^{a}, —OC(═O) R^{a}, —SH, —SR^{a}, —S(═O) R^{a}, —S(═O)₂R^{a}, —S(═O)₂NR^{c}R^{d}, —NR^{c}R^{d}, —NR^{b}C(═O)R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl.

95. The radiopharmaceutical conjugate of any one of claims 86 to 91, wherein X9 is W1Me, W, Nal1, W1Et, Nal21N, 3Bzf, 3Bzt, Nal14N, Nal18N, F23dMe, F23dC, or W1Et.

96. The radiopharmaceutical conjugate of claim 95, wherein X9 is W1Me or F23dMe.

97. The radiopharmaceutical conjugate of any one of claims 86 to 96, wherein ring A2 is a 6-membered heteroaryl containing 1 or 2 N.

98. The radiopharmaceutical conjugate of any one of claims 86 to 97, wherein R^{X5} is C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl.

99. The radiopharmaceutical conjugate of any one of claims 83 to 86, wherein
X7 is W1Me, W1MeCl, W1MeBr, Nal1, Nal2, W1Et, 3Bzf, 3Bzt, F23dC, W1Me7N, or F23dMe;
X8 is V, KCOpipzaa, Hse, N, Cit, hCit, KAc, DapAc, OrnAc, T, alT, Aib, Alb, Qglucamine, Hgl, E, Hgn, MeF, 3Py6NH2, W1Me, A, Q, or K; and
X9 is W1Me, Nal1, W1Et, Nal21N, 3Bzf, 3Bzt, Nal18N, F23dMe, or F23dC.

100. The radiopharmaceutical conjugate of claim 99, wherein X8 is KCOpipzaa, N, Cit, hCit, KAc, DapAc, OrnAc, A, T, alT, Aib, Alb, Qglucamine, Hgl, Q, E, Hgn, or K.

101. The radiopharmaceutical conjugate of claim 81 or 99, wherein
X7 is W1Me;
X8 is V; and
X9 is W1Me.

102. The radiopharmaceutical conjugate of claim 81 or 99, wherein
X7 is W1Me;
X8 is KCOpipzaa, N, Cit, hCit, KAc, DapAc, OrnAc, A, T, alT, Aib, Alb, Qglucamine, Hgl, Q, E, Hgn, or K; and
X9 is W1Me.

103. The radiopharmaceutical conjugate of any one of claims 1 to 61, wherein the peptide has an amino acid sequence according to Formula (I), or a pharmaceutically acceptable salt thereof,
X1-X2-X3-X4-X5-X6-X7-X8-X9-X10-X11-X12 Formula (I)
wherein,
X1 is any amino acid
X2 is an amino acid having an aromatic ring or a variant thereof
X3 is N,
X4 is a hydrophobic amino acid or a variant thereof;
X5 is a hydrophilic amino acid or a variant thereof;
X6 is a hydrophilic amino acid or amino acid having aromatic ring;
X7 is W or a variant thereof;
X8 is V or hydrophilic amino acid or a variant thereof,
X9 is W or a variant thereof;
X10 is T or a variant thereof;
X11 is a hydrophilic amino acid;
X12 is C or a variant thereof (such as C).

104. The radiopharmaceutical conjugate of claim 103, wherein X8 is KCOpipzaa, N, Cit, Qglucamine, hCit, K, KAc, Aib, Alb, DapAc, OrnAc, A, T, alT, Norleucine, Norvaline, Hgl, E, Hgn, Q, I, or L.

105. The radiopharmaceutical conjugate of claim 103 or 104, wherein X11 is arginine (R), asparagine (N), aspartate (D), glutamine (Q), lysine (K), or an unnatural hydrophilic amino acid.

106. The radiopharmaceutical conjugate of any one of claims 1 to 61, wherein the peptide has an amino acid sequence according to Formula (Ia), or a pharmaceutically acceptable salt thereof,
X1-X2-X3-X4-X5-X6-X7-X8-X9-X12 Formula (Ia)
wherein,
X1 is any amino acid;
X2 is an amino acid having an aromatic ring or a variant thereof;
X3 is N or a variant thereof;
X4 is a hydrophobic amino or a variant thereof,
X5 is a hydrophilic amino acid or a variant thereof;
X6 is a hydrophilic amino acid or amino acid having aromatic ring;
X7 is W or a variant thereof;
X8 is a hydrophilic amino acid or a variant thereof,
X9 is W or a variant thereof; and
X12 is C or a variant thereof.

107. The radiopharmaceutical conjugate of any one of claims 1 to 106, wherein the peptide has a monocyclic structure.

108. The radiopharmaceutical conjugate of claim 107, wherein the amino acid X1 and the cysteine or a variant thereof are bound.

109. The radiopharmaceutical conjugate of claim 107, wherein the peptide has a structure of Formula (I-1), 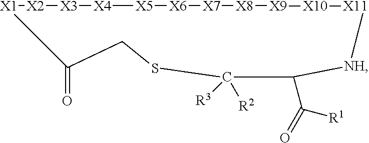
wherein
R¹ is selected from the group consisting of NH₂ and OH;
R² is selected from the group consisting of H or C₁₋₃ alkyl;
R³ is selected from the group consisting of H or C₁₋₃ alkyl;
wherein X1 to X11 have the definitions described in Formula (I), and wherein the attachment point to the radionuclide or the linker is not shown.

110. The radiopharmaceutical conjugate of claim 109, or a pharmaceutically acceptable salt thereof, wherein the peptide of Formula (I-1) has a structure of Formula (I-2),

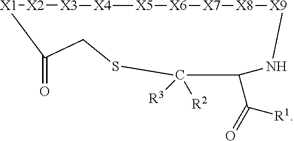

111. The radiopharmaceutical conjugate of any one of claims 22 to 110, wherein the conjugate having a structure of Formula (III-1) 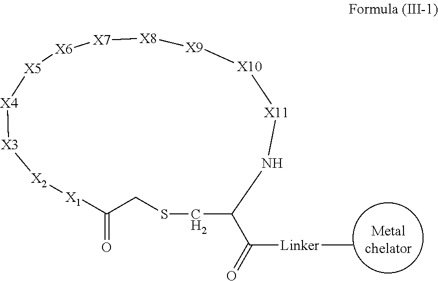
wherein X1 to X11 have the definitions described in Formula (I), and
wherein -Linker- represents the linker connecting the peptide and the metal chelator.

112. The radiopharmaceutical conjugate of any one of claims 22 to 110, wherein the conjugate having a structure of Formula (III-2), 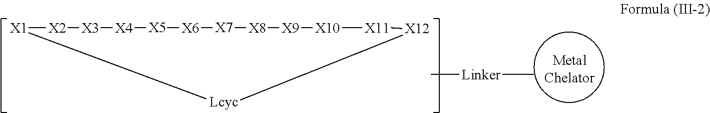
wherein
Lcyc is a ring closing group that covalently connects X1 with X12;
-Linker- represents the linker that connects the peptide and the metal chelator; and
wherein X1 to X12 have the definitions described in Formula (I).

113. The radiopharmaceutical conjugate of any one of claims 1 to 112, wherein the peptide or the salt thereof comprises an amino acid sequence that is at least 90% identical to a sequence selected from SEQ ID NOs: 1-171.

114. The radiopharmaceutical conjugate of any one of claims 1 to 112, wherein the peptide or the salt thereof consists of an amino acid sequence selected from SEQ ID NOs: 1-171.

115. The radiopharmaceutical conjugate of any one of claims 1 to 114, wherein the radiopharmaceutical conjugate is not SEQ ID NO: 282.

116. The radiopharmaceutical conjugate of any one of claims 1 to 115, wherein the peptide has a binding affinity to a human EphA2 of at most 100 nM as determined by Kd in surface plasmon resonance (SPR) analysis.

117. The radiopharmaceutical conjugate of claim 116, wherein the peptide has a binding affinity to a human EphA2 of at most 1 nM as determined by Kd in surface plasmon resonance (SPR) analysis.

118. The radiopharmaceutical conjugate of any one of claims 1 to 117, wherein the conjugate has a binding affinity to a human EphA2 of at most 100 nM as determined by Kd in surface plasmon resonance (SPR) analysis.

119. The radiopharmaceutical conjugate of claim 118, wherein the conjugate has a binding affinity to a human EphA2 of at most 1 nM as determined by Kd in surface plasmon resonance (SPR) analysis.

120. The radiopharmaceutical conjugate of any one of claims 1 to 119, wherein the conjugate has a plasma half-life (T_{1/2}) of at least 50, 100, 150, 200, 250, 300, 350, 400, 450, or 500 minutes as determined in vitro in human plasma at 37 °C.

121. The radiopharmaceutical conjugate of any one of claims 1 to 120, wherein an uptake ratio between a tumor uptake and kidney uptake toward the radiopharmaceutical conjugate is at least 1.0, 1.1, 1.2, 1.3, 1.4, 1.5, 1.6, 1.7, 1.8 or 2.0 in a human prostate xenograft mouse model.

122. The radiopharmaceutical conjugate of any one of claims 1 to 121, wherein the peptide binds to a ligand-binding domain (LBD) domain of the EphA2.

123. The radiopharmaceutical conjugate of any one of claims 1 to 122, wherein the peptide interacts with a human EphA2 at one or more amino acid residues selected from Asp53, Met55, Asn57, Met59, Met66, Thr101, Arg103, Phe156, Glu157, Arg159, Val161, Val189, and Ala190.

124. The radiopharmaceutical conjugate of any one of claims 1 to 123, wherein the peptide interacts with a human EphA2 at Asp53 and Glu157.

125. The radiopharmaceutical conjugate of any one of claim 1 or 10 to 124, wherein the peptide is a peptide of Formula (I) and wherein, when the peptide is bound to the human EphA2, amino acid residue X7 is located less than 10 Å from the Phe156 of the human EphA2.

126. The radiopharmaceutical conjugate of claim 125, wherein amino acid residue X7 is located less than 6 Å from the Phe156.

127. The radiopharmaceutical conjugate of any one of claim 1 or 10 to 126, wherein the peptide is a peptide of Formula (I) and wherein, when the peptide is bound to the human EphA2, amino acid residue X9 is located less than 10 Å from the Phe156 of the human EphA2.

128. The radiopharmaceutical conjugate of claim 127, wherein amino acid residue X9 is located less than 6 Å from the Phe156.

129. The radiopharmaceutical conjugate of any one of claim 1 or 10 to 128, wherein the peptide is a peptide of Formula (I) and wherein, when the peptide is bound to the human EphA2, amino acid residue X8 is located less than 10 Å from the Phe156 of the human EphA2.

130. The radiopharmaceutical conjugate of any one of claims 123 to 128, wherein the human EphA2 comprises a sequence of SEQ ID NO: 276 or SEQ ID NO: 277.

131. The radiopharmaceutical conjugate of claim 1, 2 or 83, wherein the conjugate is a compound of Tables 1, 2A, 2B, 2B-Lu, 2B-Lu-177, 2B-Ac-225, or 2C.

132. A radiopharmaceutical conjugate, comprising:
(a) a peptide that has avidity for ephrin type-A receptor 2 (EphA2), wherein the peptide competes for binding to a human EphA2 with a peptide that has an amino acid sequence including deletion, substitution, and/or addition of one or several amino acids in the amino acid of SEQ ID NO:1:
(SEQ ID NO: 1)
da-MeF-N-L-Hgl-MeF-W1Me-V-W1Me-T-E-C
or a pharmaceutically acceptable salt thereof; and
(b) a metal chelator configured to bind with a radionuclide, wherein the metal chelator is conjugated to the peptide.

133. The radiopharmaceutical conjugate of claim 2 or 132, wherein the 8th V is substituted.

134. The radiopharmaceutical conjugate of any one of claim 2, 132 or 133, wherein the 11th E is substituted.

135. A radiopharmaceutical conjugate, comprising:
(a) a peptide that has avidity for ephrin type-A receptor 2 (EphA2), wherein the peptide competes for binding to a human EphA2 with a peptide that has a structure of Formula (I), or a pharmaceutically acceptable salt thereof,
X1-X2-X3-X4-X5-X6-X7-X8-X9-X10-X11-X12 Formula (I)
wherein,
X1 is an amino acid;
X2 is an amino acid comprising an aromatic ring, an N-methylated amino acid thereof, or a variant thereof;
X3 is a hydrophilic amino acid (e.g. N, Q, Cit, K or a variant thereof), glycine (G), Alanine (A) or a variant thereof (e.g., da, 2-Aminoisobutyric acid (Aib));
X4 is a hydrophobic amino acid (e.g., leucine (L)), a hydrophilic amino acid (e.g., citrulline (Cit)), or a variant thereof;
X5 is a hydrophilic amino acid, or a variant thereof;
X6 is a hydrophilic amino acid, an amino acid comprising an aromatic ring, or an N-methylated amino acid thereof;
X7 is an amino acid comprising an aromatic ring (e.g., W, F, or a variant thereof);
X8 is a hydrophobic amino acid, a hydrophilic amino acid, an N-methylated amino acid, or a variant thereof;
X9 is an amino acid comprising an aromatic ring (e.g., W or a variant thereof);
X10 is absent or a hydrophilic amino acid (e.g., Threonine (T) or a variant thereof);
X11 is absent or a hydrophilic amino acid; and
X12 is cysteine (C) or a variant thereof; and
(b) a metal chelator configured to bind with a radionuclide, wherein the metal chelator is conjugated to the peptide.

136. A radiopharmaceutical conjugate comprising:
(a) a cyclic peptide that has avidity for ephrin type-A receptor 2 (EphA2), wherein the peptide consists of a sequence of Formula (I),
X1-X2-X3-X4-X5-X6-X7-X8-X9-X10-X11-X12 Formula (I)
or a pharmaceutically acceptable salt thereof,
wherein
each of X1, X2, X3, X4, X5, X6, and X8 is independently an amino acid;
X7 is W1Me or a variant thereof;
X9 is W1Me or a variant thereof;
each of X10 and X11 is independently absent or an amino acid; and
X12 is cysteine (C) or a variant thereof;
(b) a metal chelator configured to bind with a radionuclide; and
(c) a linker that connects the peptide with the metal chelator.

137. The radiopharmaceutical conjugate of any one of claim 1, 135 or 136, wherein X8 is KCOpipzaa, N, Cit, Qglucamine, hCit, K, KAc, Aib, Alb, DapAc, OrnAc, A, T, alT, Norleucine, Norvaline, Hgl, E, Hgn, Q, I, or L.

138. The radiopharmaceutical conjugate of any one of claim 1 or 135-137, wherein X11 is absent, arginine (R), asparagine (N), aspartate (D), glutamine (Q), lysine (K), or an unnatural hydrophilic amino acid.

139. The radiopharmaceutical conjugate of any one of claims 1 to 138, wherein the peptide competes for binding to a human EphA2 at one or more amino acid residues selected from Asp53, Met55, Asn57, Met59, Met66, Thr101, Arg103, Phe156, Glu157, Arg159, Val161, Val189, and Ala190.

140. The radiopharmaceutical conjugate of claim 139, wherein the peptide competes for binding to human EphA2 at one or more amino acid residues selected from Asp53, Phe156, and Glu157.

141. The radiopharmaceutical conjugate of any one of claims 132 to 140, wherein the human EphA2 comprises a sequence of SEQ ID NO: 276 or SEQ ID NO: 277.

142. A radiopharmaceutical conjugate, wherein the conjugate is a salt of a conjugate of any one of the preceding claims.

143. A pharmaceutical composition comprising a radiopharmaceutical conjugate of any one of claims 1 to 142, and a pharmaceutically acceptable excipient or carrier.

144. A radiolabeled human EphA2 protein, wherein the EphA2 protein is bound to a radiopharmaceutical conjugate of any one of claims 1 to 142.

145. A method of treating a disease or disorder characterized by overexpression of EphA2, comprising administering to the subject a radiopharmaceutical conjugate of any one of claims 1 to 142, or a pharmaceutical composition of claim 143.

146. The method of claim 145, wherein the disease or disorder is cancer.

147. A method of diagnosing or imaging a cancer in a subject in need thereof, comprising administering to the subject a radiopharmaceutical conjugate of any one of claims 1 to 142, or a pharmaceutical composition of claim 143.

148. A method of treating a cancer in a subject in need thereof, comprising administering to the subject a radiopharmaceutical conjugate of any one of claims 1 to 142, or a pharmaceutical composition of claim 143.

149. The method of claim 148, wherein the cancer is selected from glioblastoma, prostate cancer, lung cancer, breast cancer, gastric cancer, ovarian cancer, bladder cancer, colon cancer, esophageal cancer, multiple myeloma and fibrosarcoma.

150. The method of claim 148, wherein the cancer is non-small cell lung carcinomas (NSCLC).

151. The method of claim 148, wherein the cancer is triple negative breast cancer.

152. The method of claim 148 or 149, wherein the method comprises administering (i) a first radiopharmaceutical conjugate comprising a radionuclide configured for companion diagnostic (such as PET imaging) and (ii) a second radiopharmaceutical conjugate comprising a radionuclide selected from an alpha or beta-particle emitter, wherein the first and the second conjugate have the same structure except for the radionuclide.

153. The method of claim 152, wherein the radionuclide of the first conjugate is selected from Lu-177, In-111, Ga-68, Cu-64, and Zr-89.

154. A kit for use in a method of diagnosing disease or disorder characterized by an over or decreased level of expression of EphA2, wherein the kit comprising a radiopharmaceutical conjugate of any one of claims 1 to 142, or a pharmaceutical composition of claim 143.


SEQUENCE LISTING (449 sequences)

SEQ ID NO: 1 - protein, 12 aa, synthetic construct
  SITE 1: note = D-2-aminopropanoic acid
  SITE 2: note = 2-(methylamino)-3-phenylpropanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 12: note = Amidated Cysteine
  SITE 1..12: note = Optionally cyclic
AFNLXFWVWTEC

SEQ ID NO: 2 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQFWKWTQC

SEQ ID NO: 3 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQFWKWTQC

SEQ ID NO: 4 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWKWTQC

SEQ ID NO: 5 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWKWTQC

SEQ ID NO: 6 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-{1-methyl-1H-pyrrolo[2,3-b]pyridin-3-yl}p ropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQFWKWTQC

SEQ ID NO: 7 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(2,3-dimethylphenyl)propanoic acid
  SITE 11: note = 2-amino-5-carbamoylpentanoic acid
  SITE 12: note = Amidated Cysteine
  SITE 1..12: note = Optionally cyclic
AANGQFWKFTQC

SEQ ID NO: 8 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 12: note = Amidated Cysteine
  SITE 1..12: note = Optionally cyclic
AANGQFWKWTNC

SEQ ID NO: 9 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQFWKWTNC

SEQ ID NO: 10 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLXEWKWTQC

SEQ ID NO: 11 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = (S)-2-aminohexanedioic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGXEWKWTQC

SEQ ID NO: 12 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (2S)-2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 13 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQEWKWTQC

SEQ ID NO: 14 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWVWTQC

SEQ ID NO: 15 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWVWTQC

SEQ ID NO: 16 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWVWTQC

SEQ ID NO: 17 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWVWTQC

SEQ ID NO: 18 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWXWTQC

SEQ ID NO: 19 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQAWXWTQC

SEQ ID NO: 20 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWXWTQC

SEQ ID NO: 21 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQFWXWTQC

SEQ ID NO: 22 - protein, 12 aa, synthetic construct
  SITE 1: note = 2-aminoheptanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLXFWKWTQC

SEQ ID NO: 23 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 3-(4-chlorophenyl)-2-(methylamino)propanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLXXWKWTQC

SEQ ID NO: 24 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(methylcarbamoyl)propanoic acid
  SITE 12: note = Amidated Cysteine
  SITE 1..12: note = Optionally cyclic
AANLXFWKWTNC

SEQ ID NO: 25 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(dimethylcarbamoyl)propanoic acid
  SITE 12: note = Amidated Cysteine
  SITE 1..12: note = Optionally cyclic
AANLXFWKWTNC

SEQ ID NO: 26 - protein, 12 aa, synthetic construct
  SITE 1: note = 2-aminoheptanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLQEWKWTQC

SEQ ID NO: 27 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-3-(methylcarbamoyl)propanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLNEWKWTQC

SEQ ID NO: 28 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-3-(dimethylcarbamoyl)propanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLNEWKWTQC

SEQ ID NO: 29 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(methylcarbamoyl)propanoic acid
  SITE 12: note = Amidated Cysteine
  SITE 1..12: note = Optionally cyclic
AANLQEWKWTNC

SEQ ID NO: 30 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(dimethylcarbamoyl)propanoic acid
  SITE 12: note = Amidated Cysteine
  SITE 1..12: note = Optionally cyclic
AANLQEWKWTNC

SEQ ID NO: 31 - protein, 12 aa, synthetic construct
  SITE 1: note = 2-aminoheptanoic acid
  SITE 2: note = 2-(methylamino)-3-(pyridin-3-yl)propanoic acid
  SITE 4: note = 2-amino-2-cyclohexylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQFWKWTQC

SEQ ID NO: 32 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-3-(methylcarbamoyl)propanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGNFWKWTQC

SEQ ID NO: 33 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-3-(dimethylcarbamoyl)propanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGNFWKWTQC

SEQ ID NO: 34 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-5-carbamoylpentanoic acid
  SITE 6: note = 3-(4-chlorophenyl)-2-(methylamino)propanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQXWKWTQC

SEQ ID NO: 35 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(methylcarbamoyl)propanoic acid
  SITE 12: note = Amidated Cysteine
  SITE 1..12: note = Optionally cyclic
AANGQFWKWTNC

SEQ ID NO: 36 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclohexylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-3-(dimethylcarbamoyl)propanoic acid
  SITE 12: note = Amidated Cysteine
  SITE 1..12: note = Optionally cyclic
AANGQFWKWTNC

SEQ ID NO: 37 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQNWVWTQC

SEQ ID NO: 38 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNLQNWVWTQC

SEQ ID NO: 39 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNLQNWVWTQC

SEQ ID NO: 40 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQNWVWTQC

SEQ ID NO: 41 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQNWKWTQC

SEQ ID NO: 42 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQNWXWTQC

SEQ ID NO: 43 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 12: note = Amidated Cysteine
  SITE 1..12: note = Optionally cyclic
XFNGQEWXWTRC

SEQ ID NO: 44 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 12: note = Amidated Cysteine
  SITE 1..12: note = Optionally cyclic
XANGQNWXWTRC

SEQ ID NO: 45 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 12: note = Amidated Cysteine
  SITE 1..12: note = Optionally cyclic
XANGQNWXWTRC

SEQ ID NO: 46 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 12: note = Amidated Cysteine
  SITE 1..12: note = Optionally cyclic
XANGQNWKWTRC

SEQ ID NO: 47 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQFWXWTQC

SEQ ID NO: 48 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQFWKWTQC

SEQ ID NO: 49 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQFWXWTQC

SEQ ID NO: 50 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQFWKWTQC

SEQ ID NO: 51 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQAWXWTQC

SEQ ID NO: 52 - protein, 12 aa, synthetic construct
  SITE 1: note = 2-amino-3-(3-carbamoylphenyl)propanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQAWKWTQC

SEQ ID NO: 53 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQAWXWTQC

SEQ ID NO: 54 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQAWKWTQC

SEQ ID NO: 55 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQEWXWTQC

SEQ ID NO: 56 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANNGEWKWTQC

SEQ ID NO: 57 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGKFWKWTQC

SEQ ID NO: 58 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGKFWXWTQC

SEQ ID NO: 59 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNAWXWTNC

SEQ ID NO: 60 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNNWXWTNC

SEQ ID NO: 61 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-5-(carbamoylamino)pentanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNNWXWTNC

SEQ ID NO: 62 - protein, 12 aa, synthetic construct
  SITE 1: note = D-2-aminopropanoic acid
  SITE 2: note = 2-(methylamino)-3-(pyridin-3-yl)propanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLKEWKWTQC

SEQ ID NO: 63 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 64 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 12: note = Amidated Cysteine
  SITE 1..12: note = Optionally cyclic
AANLQEWKWTKC

SEQ ID NO: 65 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLKNWXWTQC

SEQ ID NO: 66 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLQNWKWTQC

SEQ ID NO: 67 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLQNWXWTKC

SEQ ID NO: 68 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLKEWKWTQC

SEQ ID NO: 69 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANLQEWKWTQC

SEQ ID NO: 70 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLQEWKWTKC

SEQ ID NO: 71 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWVWTQC

SEQ ID NO: 72 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWVWTQC

SEQ ID NO: 73 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWVWTQC

SEQ ID NO: 74 - protein, 12 aa, synthetic construct
  SITE 1: note = D-2-aminopropanoic acid
  SITE 2: note = 2-(methylamino)-3-phenylpropanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 12: note = Amidated Cysteine
  SITE 1..12: note = Optionally cyclic
AFNLXFWVWTEC

SEQ ID NO: 75 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 12: note = Amidated Cysteine
  SITE 1..12: note = Optionally cyclic
AANLXFWVWTEC

SEQ ID NO: 76 - protein, 12 aa, synthetic construct
  SITE 1: note = (2R)-2-amino-6-{[4-(carboxymethyl)piperazine-1-carb onyl]amino}hexanoic acid
  SITE 2: note = (2S)-2-(methylamino)-3-(pyridin-3-yl)propanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 12: note = Amidated Cysteine
  SITE 1..12: note = Optionally cyclic
KANLXFWVWTEC

SEQ ID NO: 77 - protein, 12 aa, synthetic construct
  SITE 1: note = (2R)-2-amino-6-{[4-(carboxymethyl)piperazine-1-carb onyl]amino}hexanoic acid
  SITE 2: note = (2S)-2-(methylamino)-3-(pyridin-3-yl)propanoic acid
  SITE 5: note = (2S)-2-amino-6-{[4-(carboxymethyl)piperazine-1-carb onyl]amino}hexanoic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 12: note = Amidated Cysteine
  SITE 1..12: note = Optionally cyclic
KANLKFWVWTEC

SEQ ID NO: 78 - protein, 12 aa, synthetic construct
  SITE 1: note = (2R)-2-amino-6-{[4-(carboxymethyl)piperazine-1-carb onyl]amino}hexanoic acid
  SITE 2: note = (2S)-2-(methylamino)-3-(pyridin-3-yl)propanoic acid
  SITE 5: note = (2S)-2-amino-6-{[4-(carboxymethyl)piperazine-1-carb onyl]amino}hexanoic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 12: note = (2S)-2-amino-5-carbamoylpentanoic acid
  SITE 12: note = Amidated Cysteine
  SITE 1..12: note = Optionally cyclic
  SITE 11: note = 2-amino-5-carbamoylpentanoic acid
KANLKFWVWTQC

SEQ ID NO: 79 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 12: note = Amidated Cysteine
  SITE 1..12: note = Optionally cyclic
AANLXFWKWTEC

SEQ ID NO: 80 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 12: note = Amidated Cysteine
  SITE 1..12: note = Optionally cyclic
AANLKFWKWTEC

SEQ ID NO: 81 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 12: note = Amidated Cysteine
  SITE 1..12: note = Optionally cyclic
AANLKFWKWTEC

SEQ ID NO: 82 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 12: note = Amidated Cysteine
  SITE 1..12: note = Optionally cyclic
AANLKFWKWTEC

SEQ ID NO: 83 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLXFWKWTQC

SEQ ID NO: 84 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLKFWKWTQC

SEQ ID NO: 85 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQFWKWTQC

SEQ ID NO: 86 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLXEWKWTQC

SEQ ID NO: 87 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLKEWKWTQC

SEQ ID NO: 88 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 89 - protein, 12 aa, synthetic construct
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 12: note = Amidated Cysteine
  SITE 1..12: note = Optionally cyclic
AANLXFWKWTEC

SEQ ID NO: 90 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQFWKWTQC

SEQ ID NO: 91 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 92 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWVWTQC

SEQ ID NO: 93 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWVWTQC

SEQ ID NO: 94 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWVWTQC

SEQ ID NO: 95 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQAWXWTQC

SEQ ID NO: 96 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = (2S)-3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQNWXWTQC

SEQ ID NO: 97 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = (2S)-2-amino-4-{[(2S,3R,4R,5R)-2,3,4,5,6 pentahydroxyhexyl] carbamoyl}butanoic acid
  SITE 6: note = (2S)-3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGXNWXWTQC

SEQ ID NO: 98 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQNWXWTQC

SEQ ID NO: 99 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQEWKWTQC

SEQ ID NO: 100 - protein, 12 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQEWKWTQC

SEQ ID NO: 101 - protein, 12 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQEWKWTQC

SEQ ID NO: 102 - protein, 12 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQAWXWTQC

SEQ ID NO: 103 - protein, 12 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQNWXWTQC

SEQ ID NO: 104 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWXWTQC

SEQ ID NO: 105 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQAWXWTQC

SEQ ID NO: 106 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWXWTQC

SEQ ID NO: 107 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNAWXWTNC

SEQ ID NO: 108 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNNWXWTNC

SEQ ID NO: 109 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-5-(carbamoylamino)pentanoic acid
  SITE 9: note = 2-amino-3-(1-methyl-1H-indol-3-yl)propanoic acid
  SITE 12: note = Amidated Cysteine
  SITE 1..12: note = Optionally cyclic
XANLNNWXWTNC

SEQ ID NO: 110 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 111 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = (2S)-3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWVWTQC

SEQ ID NO: 112 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XANGQAWXWTQC

SEQ ID NO: 113 - protein, 12 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = (2S)-2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
XFNGQEWKWTQC

SEQ ID NO: 114 - protein, 12 aa, synthetic construct
  SITE 1: note = D-2-aminopropanoic acid
  SITE 2: note = 2-(methylamino)-3-phenylpropanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 12: note = Amidated Cysteine
  SITE 1..12: note = Optionally cyclic
AFNLXFWVWTEC

SEQ ID NO: 115 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 116 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQFWKWTQC

SEQ ID NO: 117 - protein, 12 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLXEWKWTQC

SEQ ID NO: 118 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = (2S)-3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQNWVWTQC

SEQ ID NO: 119 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = (2S)-2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANGQAWVWTQC

SEQ ID NO: 120 - protein, 12 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = (2S)-3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AFNGQNWVWTQC

SEQ ID NO: 121 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQFWKWTQC

SEQ ID NO: 122 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 12: note = Amidated Cysteine
  SITE 1..12: note = Optionally cyclic
AANLQEWKWTQC

SEQ ID NO: 123 - protein, 10 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
FHNLSFWXWC

SEQ ID NO: 124 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANLSFWXWC

SEQ ID NO: 125 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANLSAWXWC

SEQ ID NO: 126 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGSAWXWC

SEQ ID NO: 127 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXAWKWC

SEQ ID NO: 128 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXAWXWC

SEQ ID NO: 129 - protein, 10 aa, synthetic construct
  SITE 1: note = 2-amino-3-(3-carbamoylphenyl)propanoic acid
  SITE 2: note = 2-amino-3-(6-aminopyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXAWKWC

SEQ ID NO: 130 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGQEWKWC

SEQ ID NO: 131 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXEWXWC

SEQ ID NO: 132 - protein, 10 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XFNGQEWKWC

SEQ ID NO: 133 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated (R)-2-amino-3-mercapto-3-methylbutanoic acid
  SITE 1..10: note = Optionally cyclic
XANGQEWKWC

SEQ ID NO: 134 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated (2R,3S)-2-amino-3-mercaptobutanoic acid
  SITE 1..10: note = Optionally cyclic
XANGQEWKWC

SEQ ID NO: 135 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated (2R,3R)-2-amino-3-mercaptobutanoic acid
  SITE 1..10: note = Optionally cyclic
XANGQEWKWC

SEQ ID NO: 136 - protein, 11 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Glycine
FHNLSFWXWCG

SEQ ID NO: 137 - protein, 10 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
FHNLSFWXWC

SEQ ID NO: 138 - protein, 10 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
FHNLSFWXWC

SEQ ID NO: 139 - protein, 10 aa, synthetic construct
  SITE 1: note = D-2-aminopropanoic acid
  SITE 2: note = 2-(methylamino)-3-phenylpropanoic acid
  SITE 4: note = 2-amino-3-cyclobutylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 10: note = Amidated Cysteine
AFNATNWXWC

SEQ ID NO: 140 - protein, 10 aa, synthetic construct
  SITE 2: note = 2-amino-3-(pyridin-4-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-3-(6-aminopyridin-3-yl)propanoic acid
  SITE 9: note = 2-amino-3-(1-methyl-1H-indol-3-yl)propanoic acid
  SITE 1..10: note = Optionally cyclic
  SITE 10: note = Amidated Cysteine
FANLEFWXWC

SEQ ID NO: 141 - protein, 10 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
FHNLSFWXWC

SEQ ID NO: 142 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANLSFWXWC

SEQ ID NO: 143 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANLSAWXWC

SEQ ID NO: 144 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANLSAWXWC

SEQ ID NO: 145 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXAWKWC

SEQ ID NO: 146 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXAWXWC

SEQ ID NO: 147 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGQEWKWC

SEQ ID NO: 148 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 10: note = Amidated Cysteine
  SITE 1..10: note = Optionally cyclic
XANGXGWXWC

SEQ ID NO: 149 - protein, 10 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 10: note = Amidated Cysteine
XFNGQEWKWC

SEQ ID NO: 150 - protein, 8 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 8: note = Amidated Cysteine
  SITE 1..8: note = Optionally cyclic
XNLSXXXC

SEQ ID NO: 151 - protein, 8 aa, synthetic construct
  SITE 2: note = 4Py2NH2
  SITE 4: note = MeA
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 8: note = Amidated Cysteine
XXLAXFXC

SEQ ID NO: 152 - protein, 8 aa, synthetic construct
  SITE 2: note = 4Py2NH2
  SITE 4: note = MeHcit
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 8: note = Amidated Cysteine
XXLAXFXC

SEQ ID NO: 153 - protein, 8 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
XXLAXFXC

SEQ ID NO: 154 - protein, 8 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
XXLAXFXC

SEQ ID NO: 155 - protein, 8 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
XXLAXFXC

SEQ ID NO: 156 - protein, 8 aa, synthetic construct
  SITE 2: note = 2-amino-3-(pyridin-4-yl)propanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
XXLGXFXC

SEQ ID NO: 157 - protein, 8 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
XXLGXFXC

SEQ ID NO: 158 - protein, 7 aa, synthetic construct
  SITE 1: note = D-2-aminopropanoic acid
  SITE 2: note = 4Py2NH2
  SITE 3: note = 2-(methylamino)-3-phenylpropanoic acid
  SITE 4: note = 2-amino-3-(1-methyl-1H-indol-3-yl)propanoic acid
  SITE 5: note = 2-(methylamino)-3-phenylpropanoic acid
  SITE 6: note = 2-amino-3-(1-methyl-1H-indol-3-yl)propanoic acid
  SITE 1..7: note = Optionally cyclic
AXFWFWC

SEQ ID NO: 159 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-{1-methyl-1H-pyrrolo[2,3-b]pyridin-3-yl}p ropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
XANGQEWKWTQC

SEQ ID NO: 160 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-{1-methyl-1H-pyrrolo[2,3-b]pyridin-3-yl}p ropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
AANGQEWKWTQC

SEQ ID NO: 161 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
AANGKEWKWTQC

SEQ ID NO: 162 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
XANGKEWKWTQC

SEQ ID NO: 163 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
AANGKEWKWTQC

SEQ ID NO: 164 - protein, 10 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(1-methyl-1H-indol-3-yl)propanoic acid
  SITE 9: note = 2-amino-5-carbamoylpentanoic acid
  SITE 1..10: note = Optionally cyclic
XANGKEWTQC

SEQ ID NO: 165 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
AANGQEWKWTKC

SEQ ID NO: 166 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
AANGKEWKWTQC

SEQ ID NO: 167 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
AANLQEWKWTEC

SEQ ID NO: 168 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
AANLQEWKWTRC

SEQ ID NO: 169 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = D-2-amino-6-(carbamoylamino)hexanoic acid
  SITE 1..12: note = Optionally cyclic
AANLQEWKWTXC

SEQ ID NO: 170 - protein, 12 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
AANLQNWKWTRC

SEQ ID NO: 171 - protein, 12 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
XANGQEWKWTRC

SEQ ID NO: 172-179 - skipped (8 entries)

SEQ ID NO: 180 - protein, 14 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine
AANGQNWVWTQCAK

SEQ ID NO: 181 - protein, 14 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine
AANGQAWVWTQCAK

SEQ ID NO: 182 - protein, 14 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine
AFNGQNWVWTQCAK

SEQ ID NO: 183 - protein, 14 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
AFNLXFWVWTECAK

SEQ ID NO: 184 - protein, 14 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
AANLQFWVWTECAK

SEQ ID NO: 185 - protein, 14 aa, synthetic construct
  SITE 1: note = 2-amino-6-{[4-(carboxymethyl)piperazine-1-carbonyl] amino}hexanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
KANLQFWVWTECAK

SEQ ID NO: 186 - protein, 14 aa, synthetic construct
  SITE 1: note = 2-amino-6-{[4-(carboxymethyl)piperazine-1-carbonyl] amino}hexanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
KANLKFWVWTECAK

SEQ ID NO: 187 - protein, 14 aa, synthetic construct
  SITE 1: note = 2-amino-6-{[4-(carboxymethyl)piperazine-1-carbonyl] amino}hexanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine
KANLKFWVWTQCAK

SEQ ID NO: 188 - protein, 14 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
AANLXFWKWTECAK

SEQ ID NO: 189 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
AANLKFWKWTECAK

SEQ ID NO: 190 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated Lysine(Ac)
AANLKFWKWTECK

SEQ ID NO: 191 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated D-Lysine(Ac)
AANLKFWKWTECK

SEQ ID NO: 192 - protein, 14 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLXFWKWTQCAK

SEQ ID NO: 193 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLKFWKWTQCAK

SEQ ID NO: 194 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLQFWKWTQCAK

SEQ ID NO: 195 - protein, 14 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLXEWKWTQCAK

SEQ ID NO: 196 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-6-{[4-(carboxymethyl)piperazine-1-carbonyl] amino}hexanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLKEWKWTQCAK

SEQ ID NO: 197 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(Ac)
AANLQEWKWTQCAK

SEQ ID NO: 198 - protein, 14 aa, synthetic construct
  SITE 2: note = 2-(methylamino)-3-(pyridin-3-yl)propanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(Ac)
AANLXFWKWTECAK

SEQ ID NO: 199 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANLQFWKWTQCK

SEQ ID NO: 200 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANLQEWKWTQCK

SEQ ID NO: 201 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANGQNWVWTQCK

SEQ ID NO: 202 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANGQAWVWTQCK

SEQ ID NO: 203 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AFNGQNWVWTQCK

SEQ ID NO: 204 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGQAWXWTQCK

SEQ ID NO: 205 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGQNWXWTQCK

SEQ ID NO: 206 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 7: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGXNWXWTQCK

SEQ ID NO: 207 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGQNWXWTQCK

SEQ ID NO: 208 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGQEWKWTQCK

SEQ ID NO: 209 - protein, 13 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XFNGQEWKWTQCK

SEQ ID NO: 210 - protein, 13 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XFNGQEWKWTQCK

SEQ ID NO: 211 - protein, 13 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XFNGQAWXWTQCK

SEQ ID NO: 212 - protein, 13 aa, synthetic construct
  SITE 1: note = 2-amino-3-(3-carbamoylphenyl)propanoic acid
  SITE 2: note = 3-(3-cyanophenyl)-2-(methylamino)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XFNGQNWXWTQCK

SEQ ID NO: 213 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AFNGQNWXWTQCK

SEQ ID NO: 214 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AFNGQAWXWTQCK

SEQ ID NO: 215 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANGQAWXWTQCK

SEQ ID NO: 216 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 1..12: note = Optionally cyclic
  SITE 13: note = Amidated D-Lysine
XANLNAWXWTNCK

SEQ ID NO: 217 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 1..12: note = Optionally cyclic
  SITE 13: note = Amidated D-Lysine
XANLNNWXWTNCK

SEQ ID NO: 218 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 3-carbamoyl-2-(methylamino)propanoic acid
  SITE 7: note = 2-amino-3-(7-chloro-1-methyl-1H-indol-3-yl)propanoi c acid
  SITE 8: note = 2-amino-5-(carbamoylamino)pentanoic acid
  SITE 9: note = 2-amino-3-(1-methyl-1H-indol-3-yl)propanoic acid
  SITE 1..12: note = Optionally cyclic
  SITE 13: note = Amidated D-Lysine
XANNLNWXWTNCK

SEQ ID NO: 219 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Lysine
AANLQEWKWTQCK

SEQ ID NO: 220 - protein, 13 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Lysine
AFNGQNWVWTQCK

SEQ ID NO: 221 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Lysine
XANGQAWXWTQCK

SEQ ID NO: 222 - protein, 13 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Lysine
XFNGQEWKWTQCK

SEQ ID NO: 223 - protein, 14 aa, synthetic construct
  SITE 1: note = D-2-aminopropanoic acid
  SITE 2: note = 2-(methylamino)-3-phenylpropanoic acid
  SITE 5: note = (S)-2-aminohexanedioic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AFNLXFXVXTECAK

SEQ ID NO: 224 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AXNLXEXXXTXCAK

SEQ ID NO: 225 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AXNLXFXXXTXCAK

SEQ ID NO: 226 - protein, 14 aa, synthetic construct
  SITE 1: note = D-2-aminopropanoic acid
  SITE 2: note = 2-(methylamino)-3-(pyridin-3-yl)propanoic acid
  SITE 5: note = (S)-2-aminohexanedioic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AXNLXEXXXTXCAK

SEQ ID NO: 227 - protein, 14 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)-3-(pyridin-3-yl)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AXNXXXXVXTXCAK

SEQ ID NO: 228 - protein, 14 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(1-amino-3,6,9,12,15,18,21,24-octa oxaheptacosan-27-oic acidc-PEG2c)
AXNXXXXVXTXCAK

SEQ ID NO: 229 - protein, 14 aa, synthetic construct
  SITE 1: note = D-2-aminopropanoic acid
  SITE 2: note = 2-(methylamino)-3-phenylpropanoic acid
  SITE 4: note = 2-amino-2-cyclobutylacetic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = 3-aminopropanoic acid
  SITE 14: note = Amidated D-Lysine(PEG8c-PEG2c)
AFNXXXXVXTXCAK

SEQ ID NO: 230 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to bA-MeG-MeG-MeG-MeG-MeG-MeG-MeG-MeG-MeG-MeG via the side chain
AXNLXFXXXTXCK

SEQ ID NO: 231 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine conjugated to bA-MeG-MeG-MeG-MeG-MeG-MeG-MeG-MeG-MeG-MeG via the side chain
AXNLXEXXXTXCK

SEQ ID NO: 232 - protein, 11 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
FHNLSFXXXCG

SEQ ID NO: 233 - protein, 11 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Lysine(Acetyl)
FHNLSFWXWCK

SEQ ID NO: 234 - protein, 11 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated D-Lysine(Acetyl)
FHNLSFWXWCK

SEQ ID NO: 235 - protein, 11 aa, synthetic construct
  SITE 1: note = D-2-aminopropanoic acid
  SITE 2: note = 2-(methylamino)-3-phenylpropanoic acid
  SITE 4: note = 2-amino-3-cyclobutylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Glycine
AFNATNWXWCG

SEQ ID NO: 236 - protein, 11 aa, synthetic construct
  SITE 2: note = 2-amino-3-(pyridin-4-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-3-(6-aminopyridin-3-yl)propanoic acid
  SITE 9: note = 2-amino-3-(1-methyl-1H-indol-3-yl)propanoic acid
  SITE 1..10: note = Optionally cyclic
  SITE 11: note = Amidated Glycine
FANLEFWXWCG

SEQ ID NO: 237 - protein, 11 aa, synthetic construct
  SITE 6: note = 2-(methylamino)-3-phenylpropanoic acid
  SITE 7: note = 2-amino-3-(1-methyl-1H-indol-3-yl)propanoic acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated D-Lysine
FHNLSFWXWCK

SEQ ID NO: 238 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-phenylpropanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANLSFWXWCK

SEQ ID NO: 239 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANLSAWXWCK

SEQ ID NO: 240 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANGSAWXWCK

SEQ ID NO: 241 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANGXAWKWCK

SEQ ID NO: 242 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)-3-(pyridin-3-yl)propanoic acid
  SITE 7: note = 2-amino-3-(7-chloro-1-methyl-1H-indol-3-yl)propanoi c acid
  SITE 8: note = 2-amino-4-{[(2S,3R,4R,5R)-2,3,4,5,6 pentahydroxyhexyl]carbamoyl}butanoic acid
  SITE 9: note = 2-amino-3-(1-methyl-1H-indol-3-yl)propanoic acid
  SITE 1..10: note = Optionally cyclic
  SITE 11: note = Amidated Lysine
XANGXAWXWCK

SEQ ID NO: 243 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANGQEWXWCK

SEQ ID NO: 244 - protein, 11 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-4-{[(2S,3R,4R,5R)-2,3,4,5,6 pentahydroxyhexyl]carbamoyl}butanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-(carbamoylamino)hexanoic acid
  SITE 9: note = 2-amino-3-(1-methyl-1H-indol-3-yl)propanoic acid
  SITE 1..10: note = Optionally cyclic
  SITE 11: note = Amidated Lysine
XANGXEWXWCK

SEQ ID NO: 245 - protein, 11 aa, synthetic construct
  SITE 1: note = 2-amino-3-(3-carbamoylphenyl)propanoic acid
  SITE 2: note = 3-(3-hydroxyphenyl)-2-(methylamino)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..10: note = Optionally cyclic
  SITE 11: note = Amidated Lysine
XFNGQEWKWCK

SEQ ID NO: 246 - protein, 9 aa, synthetic construct
  SITE 2: note = 2-amino-3-(2-aminopyridin-4-yl)propanoic acid
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated Glycine
FXLXWFWCG

SEQ ID NO: 247 - protein, 9 aa, synthetic construct
  SITE 2: note = 2-amino-6-(carbamoylamino)hexanoic acid
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated Lysine(Acetyl)
FXLXWFWCK

SEQ ID NO: 248 - protein, 9 aa, synthetic construct
  SITE 2: note = 2-amino-6-(carbamoylamino)hexanoic acid
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated D-Lysine(Acetyl)
FXLXWFWCK

SEQ ID NO: 249 - protein, 9 aa, synthetic construct
  SITE 2: note = 2-amino-3-(pyridin-4-yl)propanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated Glycine
FALGWFWCG

SEQ ID NO: 250 - protein, 9 aa, synthetic construct
  SITE 2: note = 4Py2NH2
  SITE 4: note = 2-amino-6-(carbamoylamino)hexanoic acid
  SITE 5: note = 2-amino-3-(1-methyl-1H-indol-3-yl)propanoic acid
  SITE 6: note = 2-(methylamino)-3-phenylpropanoic acid
  SITE 7: note = 2-amino-3-(1-methyl-1H-indol-3-yl)propanoic acid
  SITE 1..8: note = Optionally cyclic
  SITE 9: note = Amidated D-Lysine
FXLXWFWCK

SEQ ID NO: 251 - protein, 8 aa, synthetic construct
  SITE 1: note = D-2-aminopropanoic acid
  SITE 2: note = 2-amino-3-(2-aminopyridin-4-yl)propanoic acid
  SITE 3: note = 2-(methylamino)-3-phenylpropanoic acid
  SITE 4: note = 2-amino-3-(1-methyl-1H-indol-3-yl)propanoic acid
  SITE 5: note = 2-(methylamino)-3-phenylpropanoic acid
  SITE 6: note = 2-amino-3-(1-methyl-1H-indol-3-yl)propanoic acid
  SITE 1..7: note = Optionally cyclic
  SITE 8: note = Amidated Glycine
AXFXFXCG

SEQ ID NO: 252 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-{1-methyl-1H-pyrrolo[2,3-b]pyridin-3-yl}p ropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
XANGQEWKWTQCK

SEQ ID NO: 253 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-{1-methyl-1H-pyrrolo[2,3-b]pyridin-3-yl}p ropanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Lysine
AANGQEWKWTQCK

SEQ ID NO: 254 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = D-Glutamate
AANGKEWKWTQCE

SEQ ID NO: 255 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Glutamate
XANGKEWKWTQCE

SEQ ID NO: 256 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Glutamate
AANGKEWKWTQCE

SEQ ID NO: 257 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = (S) - 2-amino-2-cyclobutylacetic acid
  SITE 6: note = 2-(methylamino)pentanedioic acid
  SITE 7: note = 2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = Amidated D-Glutamate
  SITE 8: note = (2S)-2-amino-6-{[4-(carboxymethyl)piperazine-1-carb onyl]amino}hexanoic acid
  SITE 11: note = (2S)-2-amino-5-carbamoylpentanoic acid
XANGKEWKWTQCE

SEQ ID NO: 258 - protein, 14 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = dk
  SITE 14: note = df
AANLQEWKWTQCKF

SEQ ID NO: 259 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = D-Lys conjugated to Glycine(D-Phenylalanine) via the side chain
AANLQEWKWTQCK

SEQ ID NO: 260 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 12: note = Amidated Cysteine
  SITE 11: note = Lysine(2-amino-4,4-dimethylpentanoic acid)
AXNGQEWKWTKC

SEQ ID NO: 261 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 11: note = Lysine(D-Phenylalanine)
  SITE 12: note = Amidated Cysteine
AXNGQEWKWTKC

SEQ ID NO: 262 - protein, 12 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 11: note = Lysine(D-Glutamate)
  SITE 12: note = Amidated Cysteine
AXNGQEWKWTKC

SEQ ID NO: 263 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Glutamate
  SITE 5: note = Lysine(2-amino-4,4-dimethylpentanoic acid)
AXNGKEWKWTQCE

SEQ ID NO: 264 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Glutamate
  SITE 5: note = Lysine(D-Phenylalanine)
AXNGKEWKWTQCE

SEQ ID NO: 265 - protein, 13 aa, synthetic construct
  SITE 1: note = D-2-aminopropanoic acid
  SITE 2: note = 2-(methylamino)-3-(pyridin-3-yl)propanoic acid
  SITE 4: note = 2-amino-2-cyclobutylacetic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = 2-amino-5-carbamoylpentanoic acid
  SITE 1..12: note = Optionally cyclic
  SITE 13: note = Amidated D-Glutamate
  SITE 5: note = Lysine(D-Glutamate)
AXNGKEWKWTQCE

SEQ ID NO: 266 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 1..12: note = Optionally cyclic
  SITE 13: note = Amidated D-Lysine
AXNLXEXXXTECK

SEQ ID NO: 267 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine
AANLQEWKWTRCK

SEQ ID NO: 268 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 2-(methylamino)pentanedioic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = D-2-amino-6-(carbamoylamino)hexanoic acid
  SITE 1..12: note = Optionally cyclic
  SITE 13: note = Amidated D-Lysine
AANLQEWKWTRCK

SEQ ID NO: 269 - protein, 13 aa, synthetic construct
  SITE 1: note = D-2-aminopropanoic acid
  SITE 2: note = 2-(methylamino)-3-(pyridin-3-yl)propanoic acid
  SITE 5: note = 2-amino-5-carbamoylpentanoic acid
  SITE 6: note = 3-carbamoyl-2-(methylamino)propanoic acid
  SITE 7: note = 2-amino-3-(1-methyl-1H-indol-3-yl)propanoic acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine
AANLQEWKWTXCK

SEQ ID NO: 270 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine
AANLQNWKWTRCK

SEQ ID NO: 271 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = homo-Arginine
  SITE 1..12: note = Optionally cyclic
  SITE 13: note = D-Lysine
XANGQEWKWTRCK

SEQ ID NO: 272 - protein, 10 aa, synthetic construct
  SITE 2..4: note = 2-amino-3-(1-methyl-1H-indol-3-yl)propanoic acid
  SITE 6: note = 2-amino-3-(6-aminopyridin-3-yl)propanoic acid
  SITE 7: note = 2-(methylamino)-3-phenylpropanoic acid
  SITE 1..9: note = Optionally cyclic
  SITE 10: note = Amidated Cysteine
FWWWSXFECG

SEQ ID NO: 273 - protein, 13 aa, synthetic construct
  SITE 1: note = 2-amino-3-(3-carbamoylphenyl)propanoic acid
  SITE 2: note = 2-(methylamino)-3-(pyridin-3-yl)propanoic acid
  SITE 4: note = 2-amino-2-cyclobutylacetic acid
  SITE 5: note = 2-amino-5-carbamoylpentanoic acid
  SITE 6: note = 2-(methylamino)pentanedioic acid
  SITE 7: note = 2-amino-3-(7-chloro-1-methyl-1H-indol-3-yl)propanoi c acid
  SITE 8: note = 2-amino-6-{[4-(carboxymethyl)piperazine-1-carbonyl] amino}hexanoic acid
  SITE 9: note = 2-amino-3-(1-methyl-1H-indol-3-yl)propanoic acid
  SITE 11: note = homo-Arginine
  SITE 1..12: note = Optionally cyclic
  SITE 13: note = D-Lysine
XANGQEWKWTRCK

SEQ ID NO: 274 - protein, 13 aa, synthetic construct
KYNDRLPLYISNP

SEQ ID NO: 275 - protein, 30 aa, synthetic construct
  VARIANT 6..30: note = GGGGS repeats may be deleted
GGGGSGGGGSGGGGSGGGGSGGGGSGGGGS

SEQ ID NO: 276 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 277 - protein, 497 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKVTPRGAGLALAGPTAGDRLVT

SEQ ID NO: 278 - protein, 5 aa, synthetic construct
EAAAK

SEQ ID NO: 279 - protein, 11 aa, synthetic construct
  SITE 1: note = 3-aminopropanoic acid
  SITE 2..11: note = Methyl-Glycine
AGGGGGGGGGG

SEQ ID NO: 280 - protein, 14 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = Sarcosine
  SITE 14: note = Amidated D-Lysine(2,2',2''-(10-(1-carboxy-9,37-diox o-2,5,12,15,18,21,24,27,30,33-decaoxa-8,36-diazaoctatriaco ntan-38-yl)-1,4,7,10-tetraazacyclododecane-1,4,7-triyl)tri acetic acid(Lutetium))
AFNLXFWVWTECGK

SEQ ID NO: 281 - protein, 13 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = Amidated 2,4-Diaminobutyric acid-2,2',2''-(10-(14-c arboxy-2-oxo-6,9,12-trioxa-3-azatetradecyl)-1,4,7,10-tetra azacyclododecane-1,4,7-triyl)triacetic acid(Lutetium)
AFNLXFXVXTECX

SEQ ID NO: 282 - protein, 14 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(2,2',2''-(10-(1-carboxy-9,37-diox o-2,5,12,15,18,21,24,27,30,33-decaoxa-8,36-diazaoctatriaco ntan-38-yl)-1,4,7,10-tetraazacyclododecane-1,4,7-triyl)tri acetic acid(Lutetium))
AXNLXFXVXTECGK

SEQ ID NO: 283 - protein, 14 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(2,2',2''-(10-(39-carboxy-2,30-dio xo-6,9,12,15,18,21,24,27,34,37-decaoxa-3,31-diazanonatriac ontyl)-1,4,7,10-tetraazacyclododecane-1,4,7-triyl)triaceti c acid(Lutetium))
AFNLXFWVWTECAK

SEQ ID NO: 284 - protein, 13 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AFNLXFWVWTECK

SEQ ID NO: 285 - protein, 14 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = 3-aminopropanoic acid
  SITE 14: note = Amidated D-Lysine(PEG2-D-Lysine(2,2',2'',2'''-(1,4, 7,10-tetraazacyclododecane-1,4,7,10-tetrayl)tetraacetic acid(Lutetium))-1-amino-3,6,9,12,15,18,21,24-octaoxaheptac osan-27-oic acid-Ac)
XXNLXFXVXTXCGK

SEQ ID NO: 286 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid[Lutetium]-bA-MeG-MeG-MeG-MeG-MeG-MeG-MeG-MeG-MeG-MeG)
AXNLQFWKWTECK

SEQ ID NO: 287 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(39-carboxy-2,30-dio xo-6,9,12,15,18,21,24,27,34,37-decaoxa-3,31-diazanonatriac ontyl)-1,4,7,10-tetraazacyclododecane-1,4,7-triyl)triaceti c acid(Lutetium))
AXNLQFWKWTQCAK

SEQ ID NO: 288 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AXNLXFXXXTXCGK

SEQ ID NO: 289 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (S)-2-aminohexanedioic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(39-carboxy-2,30-dio xo-6,9,12,15,18,21,24,27,34,37-decaoxa-3,31-diazanonatriac ontyl)-1,4,7,10-tetraazacyclododecane-1,4,7-triyl)triaceti c acid(Lutetium))
AXNLQFWKWTQCGK

SEQ ID NO: 290 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(39-carboxy-2,30-dio xo-6,9,12,15,18,21,24,27,34,37-decaoxa-3,31-diazanonatriac ontyl)-1,4,7,10-tetraazacyclododecane-1,4,7-triyl)triaceti c acid(Lutetium))
AXNLQEWKWTQCAK

SEQ ID NO: 291 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid[Lutetium]-bA-MeG-MeG-MeG-MeG-MeG-MeG-MeG-MeG-MeG-MeG)
AXNLQEWKWTQCK

SEQ ID NO: 292 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AXNLQEWKWTQCK

SEQ ID NO: 293 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 1..12: note = Amidated D-Lysine(2-(4,7,10-tris(carboxymethyl)-1,4 ,7,10-tetraazacyclododecan-1-yl)pentanedioic acid(Lutetium))
AXNLQEWKWTQCK

SEQ ID NO: 294 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2''-((2S,5S,8S,11S)-10-(2-(l 2-azaneyl)-2-oxoethyl)-2,5,8,11-tetraethyl-1,4,7,10-tetraa zacyclododecane-1,4,7-triyl)triacetic acid(Lutetium))
AXNLQEWKWTQCK

SEQ ID NO: 295 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(3,6,9,15-Tetraazabicyclo[9.3.1]pe ntadeca-1(15),11,13-triene-3,6,9-triacetic acid(Lutetium))
AXNLQEWKWTQCK

SEQ ID NO: 296 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(3,6,9,15-Tetraazabicyclo[9.3.1] pentadeca-1(15),11,13-triene-4-S-(4-isothiocyanatobenzyl)- 3,6,9-triacetic acid(Lutetium))
AXNLQEWKWTQCK

SEQ ID NO: 297 - protein, 13 aa, synthetic construct
  SITE 1: note = (2R)-2-aminopropanoic acid
  SITE 3: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine[2,2',2''-(10-(2-((4-(3-((4-iminob utyl)thio)-2,5-dioxopyrrolidin-1-yl)butyl)amino)-2-oxoethy l)-1,4,7,10-tetraazacyclododecane-1,4,7-triyl)triacetic acid[Lutetium))
XXNLXFXVXTECK

SEQ ID NO: 298 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Lysine(2,2',2'',2'''-(1,4,7,10-tetraazacyclododec ane-1,4,7,10-tetrayl)tetraacetic acid(Lutetium))
AXNLQEWKWTQCK

SEQ ID NO: 299 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated MeD-Lysine(2,2',2'',2'''-(1,4,7,10-tetraaz acyclododecane-1,4,7,10-tetrayl)tetraacetic acid(Lutetium))
AXNLQEWKWTQCK

SEQ ID NO: 300 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AXNLQEWKWTRCK

SEQ ID NO: 301 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AXNLQNWKWTRCK

SEQ ID NO: 302 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AXNLQEWKWTECK

SEQ ID NO: 303 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-(carbamoylamino)hexanoic acid
  SITE 12: note = Optionally cyclic
  SITE 13: note = D-Lysine(2,2',2'',2'''-(1,4,7,10-tetraazacyclododec ane-1,4,7,10-tetrayl)tetraacetic acid(Lutetium))
AXNLQEWKWTECK

SEQ ID NO: 304 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 12: note = Cysteine-hexamethylenediamine-D-phenylalanine-2,2', 2'',2'''-(1,4,7,10-tetraazacyclododecane-1,4,7,10-tetrayl) tetraacetic acid(Lutetium)
  SITE 1..12: note = Optionally cyclic
AXNLQEWKWTQC

SEQ ID NO: 305 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 12: note = Cysteine-hexamethylenediamine-2,2',2'',2'''-(1,4,7, 10-tetraazacyclododecane-1,4,7,10-tetrayl)tetraacetic acid(Lutetium)
  SITE 1..12: note = Optionally cyclic
AXNLQEWKWTQC

SEQ ID NO: 306 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 13: note = Amidated D-Lysine(D-phenylalanine[2,2',2'',2'''-(1, 4,7,10-tetraazacyclododecane-1,4,7,10-tetrayl)tetraacetic acid(Lutetium))]
  SITE 1..12: note = Optionally cyclic
AXNLQEWKWTQCK

SEQ ID NO: 307 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 13: note = Amidated D-Lysine(Glycine-D-Phenylalanine-2,2',2'', 2'''-(1,4,7,10-tetraazacyclododecane-1,4,7,10-tetrayl)tetr aacetic acid(Lutetium))]
  SITE 1..12: note = Optionally cyclic
AXNLQEWKWTQCK

SEQ ID NO: 308 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (S)-2-amino-3-(7-chloro-1-methyl-1H-indol-3-yl)prop anoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = Lysine(2-amino-4,4-dimethylpentanoic acid[2,2',2'', 2'''-(1,4,7,10-tetraazacyclododecane-1,4,7,10-tetrayl)tetr aacetic acid(Lutetium))]
  SITE 12: note = Amidated Cysteine
  SITE 1..12: note = Optionally cyclic
AXNGQEWKWTKC

SEQ ID NO: 309 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (S)-2-amino-3-(7-chloro-1-methyl-1H-indol-3-yl)prop anoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = Lysine(D-Phenylalanine[2,2',2'',2'''-(1,4,7,10-tetr aazacyclododecane-1,4,7,10-tetrayl)tetraacetic acid(Lutetium))]]
  SITE 12: note = Amidated Cysteine
  SITE 1..12: note = Optionally cyclic
AXNGQEWKWTKC

SEQ ID NO: 310 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = Lysine(D-Glutamate[2,2',2'',2'''-(1,4,7,10-tetraaza cyclododecane-1,4,7,10-tetrayl)tetraacetic acid(Lutetium))]]
  SITE 12: note = Amidated Cysteine
  SITE 1..12: note = Optionally cyclic
AXNGQEWKWTKC

SEQ ID NO: 311 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = Lysine(2-amino-4,4-dimethylpentanoic acid[2,2',2'', 2'''-(1,4,7,10-tetraazacyclododecane-1,4,7,10-tetrayl)tetr aacetic acid(Lutetium))]]
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Glutamate
AXNGKEWKWTQCE

SEQ ID NO: 312 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = Lysine(D-Phenylalanine[2,2',2'',2'''-(1,4,7,10-tetr aazacyclododecane-1,4,7,10-tetrayl)tetraacetic acid(Lutetium)]])
  SITE 6: note = (2S)-2-(methylamino)pentanedioic acid
  SITE 7: note = (S)-2-amino-3-(7-chloro-1-methyl-1H-indol-3-yl)prop anoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Glutamate
AXNGKEWKWTQCE

SEQ ID NO: 313 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = Lysine(D-glutamate[2,2',2'',2'''-(1,4,7,10-tetraaza cyclododecane-1,4,7,10-tetrayl)tetraacetic acid(Lutetium))]]
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Glutamate
AXNGKEWKWTQCE

SEQ ID NO: 314 - protein, 14 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(1-carboxy-9,37-diox o-2,5,12,15,18,21,24,27,30,33-decaoxa-8,36-diazaoctatriaco ntan-38-yl)-1,4,7,10-tetraazacyclododecane-1,4,7-triyl)tri acetic acid(Lutetium))
AFNGQNWVWTQCAK

SEQ ID NO: 315 - protein, 14 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2'',2'''-(1,4,7,10-tetraazac yclododecane-1,4,7,10-tetrayl)tetraacetic acid(Lutetium)
AFNGQNWVWTQCAK

SEQ ID NO: 316 - protein, 13 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Lysine(2,2',2'',2'''-(1,4,7,10-tetraazacyclododec ane-1,4,7,10-tetrayl)tetraacetic acid(Lutetium))
AFNGQNWVWTQCK

SEQ ID NO: 317 - protein, 13 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AFNGQNWVWTQCK

SEQ ID NO: 318 - protein, 13 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AFNGQNWXWTQCK

SEQ ID NO: 319 - protein, 14 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(1-carboxy-9,37-diox o-2,5,12,15,18,21,24,27,30,33-decaoxa-8,36-diazaoctatriaco ntan-38-yl)-1,4,7,10-tetraazacyclododecane-1,4,7-triyl)tri acetic acid(Lutetium))
AFNGQXWXWTQCAK

SEQ ID NO: 320 - protein, 13 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AFNGQXWXWTQCK

SEQ ID NO: 321 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(1-carboxy-9,37-diox o-2,5,12,15,18,21,24,27,30,33-decaoxa-8,36-diazaoctatriaco ntan-38-yl)-1,4,7,10-tetraazacyclododecane-1,4,7-triyl)tri acetic acid(Lutetium))
AANGQAWXWTQCAK

SEQ ID NO: 322 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AANGQAWXWTQCK

SEQ ID NO: 323 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XANGQAWXWTQCK

SEQ ID NO: 324 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Lysine(2,2',2'',2'''-(1,4,7,10-tetraazacyclododec ane-1,4,7,10-tetrayl)tetraacetic acid(Lutetium))
XANGQAWXWTQCK

SEQ ID NO: 325 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XAFGQNWXWTQCK

SEQ ID NO: 326 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-4-{[(2S,3R,4R,5R)-2,3,4,5,6 pentahydroxyhexyl] carbamoyl}butanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XANGXNWXWTQCK

SEQ ID NO: 327 - protein, 14 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(1-carboxy-9,37-diox o-2,5,12,15,18,21,24,27,30,33-decaoxa-8,36-diazaoctatriaco ntan-38-yl)-1,4,7,10-tetraazacyclododecane-1,4,7-triyl)tri acetic acid(Lutetium))
XANQGEWXWTQCAK

SEQ ID NO: 328 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XANQGEWKWTQCK

SEQ ID NO: 329 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XANGQEWKWTQCK

SEQ ID NO: 330 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XANGQEWKWTRCK

SEQ ID NO: 331 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = D-Lysine(2,2',2'',2'''-(1,4,7,10-tetraazacyclododec ane-1,4,7,10-tetrayl)tetraacetic acid(Lutetium))
XANGQEWKWTRCK

SEQ ID NO: 332 - protein, 13 aa, synthetic construct
  SITE 1: note = (2R)-2-amino-3-(3-carbamoylphenyl)propanoic acid
  SITE 2: note = (2S)-3-(3-hydroxyphenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XXNGQEWKWTQCK

SEQ ID NO: 333 - protein, 13 aa, synthetic construct
  SITE 1: note = (2R)-2-amino-3-(3-carbamoylphenyl)propanoic acid
  SITE 2: note = (2S)-3-(3-hydroxyphenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Lysine(2,2',2'',2'''-(1,4,7,10-tetraazacyclododec ane-1,4,7,10-tetrayl)tetraacetic acid(Lutetium))
XXNGQEWKWTQCK

SEQ ID NO: 334 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 5: note = Lysine(2,2',2'',2'''-(1,4,7,10-tetraazacyclododecan e-1,4,7,10-tetrayl)tetraacetic acid(Lutetium))
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 12: note = Amidated Cysteine
  SITE 1..12: note = Optionally cyclic
AXNLKEWKWTQC

SEQ ID NO: 335 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = Lysine(2,2',2'',2'''-(1,4,7,10-tetraazacyclododecan e-1,4,7,10-tetrayl)tetraacetic acid(Lutetium))
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
XXNLKNWXWTQC

SEQ ID NO: 336 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = Lysine(2,2',2'',2'''-(1,4,7,10-tetraazacyclododecan e-1,4,7,10-tetrayl)tetraacetic acid(Lutetium))
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
XANLKEWKWTQC

SEQ ID NO: 337 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = Lysine(2,2',2'',2'''-(1,4,7,10-tetraazacyclododecan e-1,4,7,10-tetrayl)tetraacetic acid(Lutetium))
  SITE 9: note = (2S)-2-amino-3-(1-methyl-1H-indol-3-yl)propanoic acid
  SITE 11: note = (2S)-2-amino-5-carbamoylpentanoic acid
  SITE 12: note = Amidated Cysteine
  SITE 1..12: note = Optionally cyclic
AANLQEWKWTQC

SEQ ID NO: 338 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = Lysine(2,2',2'',2'''-(1,4,7,10-tetraazacyclododecan e-1,4,7,10-tetrayl)tetraacetic acid(Lutetium))
  SITE 9: note = (2S)-2-amino-3-(1-methyl-1H-indol-3-yl)propanoic acid
  SITE 11: note = (2S)-2-amino-5-carbamoylpentanoic acid
  SITE 12: note = Amidated Cysteine
  SITE 1..12: note = Optionally cyclic
XANLQNWKWTQC

SEQ ID NO: 339 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = Lysine(2,2',2'',2'''-(1,4,7,10-tetraazacyclododecan e-1,4,7,10-tetrayl)tetraacetic acid(Lutetium))
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
XANLQEWKWTQC

SEQ ID NO: 340 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = Lysine(2,2',2'',2'''-(1,4,7,10-tetraazacyclododecan e-1,4,7,10-tetrayl)tetraacetic acid(Lutetium))
  SITE 1..12: note = Optionally cyclic
  SITE 12: note = Amidated Cysteine
AANLQEWKWTKC

SEQ ID NO: 341 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = Lysine(2,2',2'',2'''-(1,4,7,10-tetraazacyclododecan e-1,4,7,10-tetrayl)tetraacetic acid(Lutetium))
  SITE 1..12: note = Optionally cyclic
  SITE 12: note = Amidated Cysteine
XANLQNWXWTKC

SEQ ID NO: 342 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = Lysine(2,2',2'',2'''-(1,4,7,10-tetraazacyclododecan e-1,4,7,10-tetrayl)tetraacetic acid(Lutetium))
  SITE 1..12: note = Optionally cyclic
  SITE 12: note = Amidated Cysteine
XANLQEWKWTKC

SEQ ID NO: 343 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AANGQNWVWTQCK

SEQ ID NO: 344 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(1-carboxy-9,37-diox o-2,5,12,15,18,21,24,27,30,33-decaoxa-8,36-diazaoctatriaco ntan-38-yl)-1,4,7,10-tetraazacyclododecane-1,4,7-triyl)tri acetic acid(Lutetium))
AANGQNWVWTQCAK

SEQ ID NO: 345 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
AANGQAWVWTQCK

SEQ ID NO: 346 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(1-carboxy-9,37-diox o-2,5,12,15,18,21,24,27,30,33-decaoxa-8,36-diazaoctatriaco ntan-38-yl)-1,4,7,10-tetraazacyclododecane-1,4,7-triyl)tri acetic acid(Lutetium))
AANGQAWVWTQCAK

SEQ ID NO: 347 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XANGQNWXWTQCK

SEQ ID NO: 348 - protein, 13 aa, synthetic construct
  SITE 1: note = (2R)-2-amino-3-(3-carbamoylphenyl)propanoic acid
  SITE 2: note = (2S)-3-(3-cyanophenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XXNGQEWKWTQCK

SEQ ID NO: 349 - protein, 13 aa, synthetic construct
  SITE 1: note = (2R)-2-amino-3-(3-carbamoylphenyl)propanoic acid
  SITE 2: note = (2S)-3-(3-cyanophenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XXNGQAWXWTQCK

SEQ ID NO: 350 - protein, 13 aa, synthetic construct
  SITE 1: note = (2R)-2-amino-3-(3-carbamoylphenyl)propanoic acid
  SITE 2: note = (2S)-3-(3-cyanophenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XXNGQNWXWTQCK

SEQ ID NO: 351 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XANLNAWVWTNCK

SEQ ID NO: 352 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XANLNNWVWTNCK

SEQ ID NO: 353 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = Citrulline
  SITE 9: note = (2S)-2-amino-3-(1-methyl-1H-indol-3-yl)propanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid(Lutetium))
XANLNNWXWTNCK

SEQ ID NO: 354 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2'',2'''-(1,4,7,10-tetraazac yclododecane-1,4,7,10-tetrayl)tetraacetic acid-(Lutetium))
XANGQNWVWTQCAK

SEQ ID NO: 355 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2'',2'''-(1,4,7,10-tetraazac yclododecane-1,4,7,10-tetrayl)tetraacetic acid(Lutetium))
AANGQAWVWTQCAK

SEQ ID NO: 356 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = Lysine(2,2',2'',2'''-(1,4,7,10-tetraazacyclododecan e-1,4,7,10-tetrayl)tetraacetic acid(Lutetium))
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Glutamate
XANGKEWKWTQCE

SEQ ID NO: 357 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = Lysine(2,2',2'',2'''-(1,4,7,10-tetraazacyclododecan e-1,4,7,10-tetrayl)tetraacetic acid(Lutetium))
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Glutamate
XANGKEWKWTQCE

SEQ ID NO: 358 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = Lysine(2,2',2'',2'''-(1,4,7,10-tetraazacyclododecan e-1,4,7,10-tetrayl)tetraacetic acid(Lutetium))
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Glutamate
XANGKEWKWTQCE

SEQ ID NO: 359 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = Lysine(2,2',2'',2'''-(1,4,7,10-tetraazacyclododecan e-1,4,7,10-tetrayl)tetraacetic acid(Lutetium))
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Glutamate
AANGKEWKWTQCE

SEQ ID NO: 360 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = Lysine(2,2',2'',2'''-(1,4,7,10-tetraazacyclododecan e-1,4,7,10-tetrayl)tetraacetic acid(Lutetium))
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
XANGKEWKWTQC

SEQ ID NO: 361 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = Lysine(2,2',2'',2'''-(1,4,7,10-tetraazacyclododecan e-1,4,7,10-tetrayl)tetraacetic acid(Lutetium)
  SITE 1..12: note = Optionally cyclic
  SITE 12: note = Amidated Cysteine
XANLQEWKWTKC

SEQ ID NO: 362 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 6: note = (2S)-2-(methylamino)-3-phenylpropanoic acid
  SITE 7: note = (2S)-2-amino-3-(7-chloro-1-methyl-1H-indol-3-yl)pro panoic acid
  SITE 8: note = (2S)-2-amino-6-(carbamoylamino)hexanoic acid
  SITE 9: note = (2S)-2-amino-3-(1-methyl-1H-indol-3-yl)propanoic acid
  SITE 1..10: note = Optionally cyclic
  SITE 11: note = Amidated Lysine(2,2',2'',2'''-(1,4,7,10-tetraazacyc lododecane-1,4,7,10-tetrayl)tetraacetic acid)
XANLSFWXWCK

SEQ ID NO: 363 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-6-(carbamoylamino)hexanoic acid
  SITE 9: note = (2S)-2-amino-3-(1-methyl-1H-indol-3-yl)propanoic acid
  SITE 1..10: note = Optionally cyclic
  SITE 11: note = Amidated Lysine(2,2',2'',2'''-(1,4,7,10-tetraazacyc lododecane-1,4,7,10-tetrayl)tetraacetic acid)
XANLSAWXWCK

SEQ ID NO: 364 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-6-(carbamoylamino)hexanoic acid
  SITE 9: note = (2S)-2-amino-3-(1-methyl-1H-indol-3-yl)propanoic acid
  SITE 1..10: note = Optionally cyclic
  SITE 11: note = Amidated Lysine(2,2',2'',2'''-(1,4,7,10-tetraazacyc lododecane-1,4,7,10-tetrayl)tetraacetic acid)
XANGSAWXWCK

SEQ ID NO: 365 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-4-{[(2S,3R,4R,5R)-2,3,4,5,6 pentahydroxyhexyl] carbamoyl}butanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 1..10: note = Optionally cyclic
  SITE 11: note = Amidated Lysine(2,2',2'',2'''-(1,4,7,10-tetraazacyc lododecane-1,4,7,10-tetrayl)tetraacetic acid)
XANGXAWKWCK

SEQ ID NO: 366 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-4-{[(2S,3R,4R,5R)-2,3,4,5,6 pentahydroxyhexyl] carbamoyl}butanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 1..10: note = Optionally cyclic
  SITE 11: note = Amidated Lysine(2,2',2'',2'''-(1,4,7,10-tetraazacyc lododecane-1,4,7,10-tetrayl)tetraacetic acid)
XANGXAWXWCK

SEQ ID NO: 367 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 1..10: note = Optionally cyclic
  SITE 11: note = Amidated Lysine(2,2',2'',2'''-(1,4,7,10-tetraazacyc lododecane-1,4,7,10-tetrayl)tetraacetic acid)
XANGQEWKWCK

SEQ ID NO: 368 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-4-{[(2S,3R,4R,5R)-2,3,4,5,6 pentahydroxyhexyl] carbamoyl}butanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-(carbamoylamino)hexanoic acid
  SITE 9: note = (2S)-2-amino-3-(1-methyl-1H-indol-3-yl)propanoic acid
  SITE 1..10: note = Optionally cyclic
  SITE 11: note = Amidated Lysine(2,2',2'',2'''-(1,4,7,10-tetraazacyc lododecane-1,4,7,10-tetrayl)tetraacetic acid)
XANGXEWXWCK

SEQ ID NO: 369 - protein, 11 aa, synthetic construct
  SITE 1: note = (2R)-2-amino-3-(3-carbamoylphenyl)propanoic acid
  SITE 2: note = (2S)-3-(3-hydroxyphenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 1..10: note = Optionally cyclic
  SITE 11: note = Amidated Lysine(2,2',2'',2'''-(1,4,7,10-tetraazacyc lododecane-1,4,7,10-tetrayl)tetraacetic acid)
XFNGQEWKWCK

SEQ ID NO: 370 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(49-carboxy-2,30,40- trioxo-6,9,12,15,18,21,24,27,34,37,44,47-dodecaoxa-3,31,41 -triazanonatetracontyl)-1,4,7,10-tetraazacyclododecane-1,4 ,7-triyl)triacetic acid)
AANLQEWKWTQCAK

SEQ ID NO: 371 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(49-carboxy-2,30,40- trioxo-6,9,12,15,18,21,24,27,34,37,44,47-dodecaoxa-3,31,41 -triazanonatetracontyl)-1,4,7,10-tetraazacyclododecane-1,4 ,7-triyl)triacetic acid)
AANLQFWKWTQCAK

SEQ ID NO: 372 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (S)-2-aminohexanedioic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(49-carboxy-2,30,40- trioxo-6,9,12,15,18,21,24,27,34,37,44,47-dodecaoxa-3,31,41 -triazanonatetracontyl)-1,4,7,10-tetraazacyclododecane-1,4 ,7-triyl)triacetic)
AANLXEWKWTQCAK

SEQ ID NO: 373 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
AANLQEWKWTQCK

SEQ ID NO: 374 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid-bA-MeG-MeG-MeG-MeG-MeG-MeG-MeG-MeG-MeG-MeG)
AANLQFWKWTQCK

SEQ ID NO: 375 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Lysine(2,2',2'',2'''-(1,4,7,10-tetraazacyclododec ane-1,4,7,10-tetrayl)tetraacetic acid)
AANLQFWKWTQCK

SEQ ID NO: 376 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Lysine(2,2',2'',2'''-(1,4,7,10-tetraazacyclododec ane-1,4,7,10-tetrayl)tetraacetic acid-bA-MeG-MeG-MeG-MeG-MeG-MeG-MeG-MeG-MeG-MeG)
AANLQEWKWTQCK

SEQ ID NO: 377 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2'',2'''-(1,4,7,10-tetraazac yclododecane-1,4,7,10-tetrayl)tetraacetic acid)
AANLQEWKWTQCAK

SEQ ID NO: 378 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2'',2'''-(1,4,7,10-tetraazac yclododecane-1,4,7,10-tetrayl)tetraacetic acid)
AANGQNWVWTQCAK

SEQ ID NO: 379 - protein, 14 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2'',2'''-(1,4,7,10-tetraazac yclododecane-1,4,7,10-tetrayl)tetraacetic acid)
AANGQNWVWTQCAK

SEQ ID NO: 380 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(49-carboxy-2,30,40- trioxo-6,9,12,15,18,21,24,27,34,37,44,47-dodecaoxa-3,31,41 -triazanonatetracontyl)-1,4,7,10-tetraazacyclododecane-1,4 ,7-triyl)triacetic acid)
AANGQNWVWTQCAK

SEQ ID NO: 381 - protein, 14 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(49-carboxy-2,30,40- trioxo-6,9,12,15,18,21,24,27,34,37,44,47-dodecaoxa-3,31,41 -triazanonatetracontyl)-1,4,7,10-tetraazacyclododecane-1,4 ,7-triyl)triacetic acid)
AANGQAWVWTQCAK

SEQ ID NO: 382 - protein, 14 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-aminopropanoic acid
  SITE 14: note = Amidated D-Lysine(2,2',2''-(10-(49-carboxy-2,30,40- trioxo-6,9,12,15,18,21,24,27,34,37,44,47-dodecaoxa-3,31,41 -triazanonatetracontyl)-1,4,7,10-tetraazacyclododecane-1,4 ,7-triyl)triacetic acid)
AANGQNWVWTQCAK

SEQ ID NO: 383 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
AANGQNWVWTQCK

SEQ ID NO: 384 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
AANGQAWVWTQCK

SEQ ID NO: 385 - protein, 13 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
AFNGQNWVWTQCK

SEQ ID NO: 386 - protein, 13 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
AFNGQNWXWTQCK

SEQ ID NO: 387 - protein, 13 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
AFNGQAWXWTQCK

SEQ ID NO: 388 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(1-methyl-1H-indol-3-yl)propanoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
AANGQAWXWTQCK

SEQ ID NO: 389 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine
XANGQAWXWTQCK

SEQ ID NO: 390 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XANGQNWXWTQCK

SEQ ID NO: 391 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-4-{[(2S,3R,4R,5R)-2,3,4,5,6 pentahydroxyhexyl] carbamoyl}butanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XANGXNWXWTQCK

SEQ ID NO: 392 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XANGQNWXWTQCK

SEQ ID NO: 393 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XANGQEWKWTQCK

SEQ ID NO: 394 - protein, 13 aa, synthetic construct
  SITE 1: note = (2R)-2-amino-3-(3-carbamoylphenyl)propanoic acid
  SITE 2: note = (2S)-3-(3-cyanophenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XFNGQEWKWTQCK

SEQ ID NO: 395 - protein, 13 aa, synthetic construct
  SITE 1: note = (2R)-2-amino-3-(3-carbamoylphenyl)propanoic acid
  SITE 2: note = (2S)-3-(3-hydroxyphenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XFNGQEWKWTQCK

SEQ ID NO: 396 - protein, 13 aa, synthetic construct
  SITE 1: note = (2R)-2-amino-3-(3-carbamoylphenyl)propanoic acid
  SITE 2: note = (2S)-3-(3-cyanophenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XFNGQAWXWTQCK

SEQ ID NO: 397 - protein, 13 aa, synthetic construct
  SITE 1: note = (2R)-2-amino-3-(3-carbamoylphenyl)propanoic acid
  SITE 2: note = (2S)-3-(3-cyanophenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XFNGQNWXWTQCK

SEQ ID NO: 398 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XANLNAWXWTNCK

SEQ ID NO: 399 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XANLNNWXWTNCK

SEQ ID NO: 400 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = Citrulline
  SITE 9: note = (2S)-2-amino-3-(1-methyl-1H-indol-3-yl)propanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XANLNNWXWTNCK

SEQ ID NO: 401 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 5: note = Lysine(2,2',2'',2'''-(1,4,7,10-tetraazacyclododecan e-1,4,7,10-tetrayl)tetraacetic acid)
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
AANLKEWKWTQC

SEQ ID NO: 402 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = Lysine(2,2',2'',2'''-(1,4,7,10-tetraazacyclododecan e-1,4,7,10-tetrayl)tetraacetic acid)
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
AANLQEWKWTQC

SEQ ID NO: 403 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = Lysine(2,2',2'',2'''-(1,4,7,10-tetraazacyclododecan e-1,4,7,10-tetrayl)tetraacetic acid)
  SITE 1..12: note = Optionally cyclic
  SITE 12: note = Amidated Cysteine
AANLQEWKWTKC

SEQ ID NO: 404 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = Lysine(2,2',2'',2'''-(1,4,7,10-tetraazacyclododecan e-1,4,7,10-tetrayl)tetraacetic acid)
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
XANLKNWXWTQC

SEQ ID NO: 405 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = Lysine(2,2',2'',2'''-(1,4,7,10-tetraazacyclododecan e-1,4,7,10-tetrayl)tetraacetic acid)
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
XANLQNWKWTQC

SEQ ID NO: 406 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = Lysine(2,2',2'',2'''-(1,4,7,10-tetraazacyclododecan e-1,4,7,10-tetrayl)tetraacetic acid)
  SITE 1..12: note = Optionally cyclic
  SITE 12: note = Amidated Cysteine
XANLQNWXWTKC

SEQ ID NO: 407 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = Lysine(2,2',2'',2'''-(1,4,7,10-tetraazacyclododecan e-1,4,7,10-tetrayl)tetraacetic acid)
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
XANLKEWKWTQC

SEQ ID NO: 408 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = Lysine(2,2',2'',2'''-(1,4,7,10-tetraazacyclododecan e-1,4,7,10-tetrayl)tetraacetic acid)
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
XANLQEWKWTQC

SEQ ID NO: 409 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = Lysine(2,2',2'',2'''-(1,4,7,10-tetraazacyclododecan e-1,4,7,10-tetrayl)tetraacetic acid)
  SITE 1..12: note = Optionally cyclic
  SITE 12: note = Amidated Cysteine
XANLQEWKWTQC

SEQ ID NO: 410 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Lysine(2,2',2'',2'''-(1,4,7,10-tetraazacyclododec ane-1,4,7,10-tetrayl)tetraacetic acid)
AANLQEWKWTQCK

SEQ ID NO: 411 - protein, 13 aa, synthetic construct
  SITE 1: note = (2R)-2-aminopropanoic acid
  SITE 2: note = (2S)-2-(methylamino)-3-phenylpropanoic acid
  SITE 4: note = (S) - 2-amino-2-cyclobutylacetic acid
  SITE 5: note = (2S)-2-amino-5-carbamoylpentanoic acid
  SITE 6: note = (2S)-3-carbamoyl-2-(methylamino)propanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Lysine(2,2',2'',2'''-(1,4,7,10-tetraazacyclododec ane-1,4,7,10-tetrayl)tetraacetic acid)
AFNGQNWVWTQCK

SEQ ID NO: 412 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Lysine(2,2',2'',2'''-(1,4,7,10-tetraazacyclododec ane-1,4,7,10-tetrayl)tetraacetic acid)
XANGQAWXWTQCK

SEQ ID NO: 413 - protein, 13 aa, synthetic construct
  SITE 1: note = (2R)-2-amino-3-(3-carbamoylphenyl)propanoic acid
  SITE 2: note = (2S)-3-(3-hydroxyphenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = D-Lysine(2,2',2'',2'''-(1,4,7,10-tetraazacyclododec ane-1,4,7,10-tetrayl)tetraacetic acid)
XFNGQEWKWTQCK

SEQ ID NO: 414 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
AANLQEWKWTRCK

SEQ ID NO: 415 - protein, 4 aa, synthetic construct
  SITE 1: note = Lysine(4-(N-maleimidomethyl)-cyclohexane-1-carbonyl )
  SITE 2: note = Lysine(1-((4-thiocarbonylamino)benzyl)-DTPA)
  SITE 3: note = D-Tyrosine
  SITE 4: note = Lysine(1-((4-thiocarbonylamino)benzyl)-DTPA)
KKYK

SEQ ID NO: 416 - protein, 13 aa, synthetic construct
  SITE 1: note = ClAc D-Alanine
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine
XANGQAWXWTQCK

SEQ ID NO: 417 - protein, 13 aa, synthetic construct
  SITE 1: note = D-Alanine-C(O)CH2Cl
  SITE 2: note = (2S)-2-(methylamino)-3-(pyridin-3-yl)propanoic acid
  SITE 3: note = Asparagine(Triphenylmethyl)
  SITE 5: note = (2S)-2-amino-5-carbamoylpentanoic acid(Triphenylmethyl)
  SITE 6: note = Me-Glutamic acid(Tert-butyl)
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid(Tert-butyl)
  SITE 9: note = (2S)-2-amino-3-(1-methyl-1H-indol-3-yl)propanoic acid
  SITE 10: note = Threonine(Tert-butyl)
  SITE 11: note = Homo-Arginine(Pbf)
  SITE 12: note = Cysteine(Trt)
  SITE 13: note = Amidated D-Lysine(Tert-butyloxycarbonyl)
AANLQEWKWTRCK

SEQ ID NO: 418 - protein, 13 aa, synthetic construct
  SITE 1: note = da-C(O)CH2Cl
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-carbamimidamidohexanoic acid
  SITE 13: note = Amidated D-Lysine
AANLQEWKWTRCK

SEQ ID NO: 419 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine
XANGQAWXWTQCK

SEQ ID NO: 420 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XANGQAWXWTQCK

SEQ ID NO: 421 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid-Lu)
XANGQAWXWTQCK

SEQ ID NO: 422 - protein, 13 aa, synthetic construct
  SITE 1: note = (2R)-2-aminopropanoic acid
  SITE 2: note = (2S)-2-(methylamino)-3-phenylpropanoic acid
  SITE 5: note = (2S)-2-amino-5-carbamoylpentanoic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = A3-aminopropanoic acid
  SITE 13: note = Amidated D-Lysine(2-(2-(2-aminoethoxy)ethoxy)acetic acid-PEG8-Biotin)
XANGQAWXWTQCK

SEQ ID NO: 423 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated Glycine(PEG10)Lysine(Biotin)
XANGQAWXWTQCG

SEQ ID NO: 424 - protein, 14 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = A3-aminopropanoic acid
  SITE 14: note = Amidated D-Lysine(2-(2-Aminoethoxy)ethanol-1-amino- 3,6,9,12,15,18,21,24-octaoxaheptacosan-27-oic acid-2,2',2' ',2'''-(1,4,7,10-tetraazacyclododecane-1,4,7,10-tetrayl)te traacetic acid)
AFNLXFWVWTECAK

SEQ ID NO: 425 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-4-{[(2S,3R,4R,5R)-2,3,4,5,6 pentahydroxyhexyl] carbamoyl}butanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 1..10: note = Optionally cyclic
  SITE 11: note = Amidated D-Lysine(2,2',2'',2'''-(1,4,7,10-tetraazac yclododecane-1,4,7,10-tetrayl)tetraacetic acid(Lutetium))
XANGXAWKWCK

SEQ ID NO: 426 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 6: note = (2S)-2-(methylamino)-3-phenylpropanoic acid
  SITE 7: note = (2S)-2-amino-3-(7-chloro-1-methyl-1H-indol-3-yl)pro panoic acid
  SITE 8: note = (2S)-2-amino-6-(carbamoylamino)hexanoic acid
  SITE 9: note = (2S)-2-amino-3-(1-methyl-1H-indol-3-yl)propanoic acid
  SITE 1..10: note = Optionally cyclic
  SITE 11: note = Amidated K(2,2',2'',2'''-(1,4,7,10-tetraazacyclodod ecane-1,4,7,10-tetrayl)tetraacetic acid-(Lutetium))
XANLSFWXWCK

SEQ ID NO: 427 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-6-(carbamoylamino)hexanoic acid
  SITE 9: note = (2S)-2-amino-3-(1-methyl-1H-indol-3-yl)propanoic acid
  SITE 1..10: note = Optionally cyclic
  SITE 11: note = Amidated K(2,2',2'',2'''-(1,4,7,10-tetraazacyclodod ecane-1,4,7,10-tetrayl)tetraacetic acid-(Lutetium))
XANLSAWXWCK

SEQ ID NO: 428 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-6-(carbamoylamino)hexanoic acid
  SITE 9: note = (2S)-2-amino-3-(1-methyl-1H-indol-3-yl)propanoic acid
  SITE 1..10: note = Optionally cyclic
  SITE 11: note = Amidated K(2,2',2'',2'''-(1,4,7,10-tetraazacyclodod ecane-1,4,7,10-tetrayl)tetraacetic acid-(Lutetium))
XANGSAWXWCK

SEQ ID NO: 429 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-4-{[(2S,3R,4R,5R)-2,3,4,5,6 pentahydroxyhexyl] carbamoyl}butanoic acid
  SITE 6: note = (2S)-2-(methylamino)-3-(pyridin-3-yl)propanoic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 1..10: note = Optionally cyclic
  SITE 11: note = Amidated K(2,2',2'',2'''-(1,4,7,10-tetraazacyclodod ecane-1,4,7,10-tetrayl)tetraacetic acid-(Lutetium))
XANGXAWXWCK

SEQ ID NO: 430 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 1..10: note = Optionally cyclic
  SITE 11: note = Amidated K(2,2',2'',2'''-(1,4,7,10-tetraazacyclodod ecane-1,4,7,10-tetrayl)tetraacetic acid-(Lutetium))
XANGQEWKWCK

SEQ ID NO: 431 - protein, 11 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-4-{[(2S,3R,4R,5R)-2,3,4,5,6 pentahydroxyhexyl] carbamoyl}butanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-(carbamoylamino)hexanoic acid
  SITE 9: note = (2S)-2-amino-3-(1-methyl-1H-indol-3-yl)propanoic acid
  SITE 1..10: note = Optionally cyclic
  SITE 11: note = Amidated K(2,2',2'',2'''-(1,4,7,10-tetraazacyclodod ecane-1,4,7,10-tetrayl)tetraacetic acid-(Lutetium))
XANGXEWXWCK

SEQ ID NO: 432 - protein, 11 aa, synthetic construct
  SITE 1: note = (2R)-2-amino-3-(3-carbamoylphenyl)propanoic acid
  SITE 2: note = (2S)-3-(3-hydroxyphenyl)-2-(methylamino)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 1..10: note = Optionally cyclic
  SITE 11: note = Amidated K(2,2',2'',2'''-(1,4,7,10-tetraazacyclodod ecane-1,4,7,10-tetrayl)tetraacetic acid-(Lutetium))
XFNGQEWKWCK

SEQ ID NO: 433 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-6-carbamimidamidohexanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XANGQEWKWTRCK

SEQ ID NO: 434 - protein, 13 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 4: note = (S) - 2-amino-2-cyclobutylacetic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-4-{[(2S,3R,4R,5R)-2,3,4,5,6 pentahydroxyhexyl] carbamoyl}butanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = Amidated D-Lysine(2,2',2'',2'''-(1,4,7,10-tetraazac yclododecane-1,4,7,10-tetrayl)tetraacetic acid)
XANQGEWKWTQCK

SEQ ID NO: 435 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 5: note = Lysine(2,2',2'',2'''-(1,4,7,10-tetraazacyclododecan e-1,4,7,10-tetrayl)tetraacetic acid)
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 12: note = Amidated Cysteine
  SITE 1..12: note = Optionally cyclic
AXNLKEWKWTQC

SEQ ID NO: 436 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = Lysine(2,2',2'',2'''-(1,4,7,10-tetraazacyclododecan e-1,4,7,10-tetrayl)tetraacetic acid)
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
AANGKEWKWTQC

SEQ ID NO: 437 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = Lysine(2,2',2'',2'''-(1,4,7,10-tetraazacyclododecan e-1,4,7,10-tetrayl)tetraacetic acid)
  SITE 12: note = Amidated Cysteine
  SITE 1..12: note = Optionally cyclic
XANLQEWKWTKC

SEQ ID NO: 438 - protein, 12 aa, synthetic construct
  SITE 1: note = (2R)-2-aminopropanoic acid
  SITE 2: note = (2S)-2-(methylamino)-3-(pyridin-3-yl)propanoic acid
  VARIANT 5: note = Any amino acid with radionuclide linker side chain
  SITE 6: note = (2S)-2-(methylamino)pentanedioic acid
  SITE 7: note = (2S)-2-amino-3-(7-chloro-1-methyl-1H-indol-3-yl)pro panoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 12: note = Amidated Cysteine
  SITE 1..12: note = Optionally cyclic
AXNLKEWKWTQC

SEQ ID NO: 439 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  VARIANT 8: note = Any amino acid with radionuclide linker side chain
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 12: note = Amidated Cysteine
AANGKEWKWTQC

SEQ ID NO: 440 - protein, 12 aa, synthetic construct
  SITE 1: note = (2R)-2-amino-3-(3-carbamoylphenyl)propanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(7-chloro-1-methyl-1H-indol-3-yl)pro panoic acid
  VARIANT 8: note = Any amino acid with radionuclide linker side chain
  SITE 9: note = (2S)-2-amino-3-(1-methyl-1H-indol-3-yl)propanoic acid
  SITE 1..12: note = Optionally cyclic
  SITE 12: note = Amidated Cysteine
XANLQEWKWTKC

SEQ ID NO: 441 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = 3-iodoTyrosine
AANLQEWKWTQCY

SEQ ID NO: 442 - protein, 12 aa, synthetic construct
  SITE 1: note = (2R)-2-aminopropanoic acid
  SITE 2: note = (2S)-2-(methylamino)-3-(pyridin-3-yl)propanoic acid
  SITE 5: note = 3-iodoTyrosine
  SITE 6: note = (2S)-2-(methylamino)pentanedioic acid
  SITE 7: note = (2S)-2-amino-3-{1-methyl-1H-pyrrolo[2,3-b]pyridin-3 -yl}propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
AANLYEWKWTQC

SEQ ID NO: 443 - protein, 13 aa, synthetic construct
  SITE 1: note = (2R)-2-aminopropanoic acid
  SITE 2: note = (2S)-2-(methylamino)-3-(pyridin-3-yl)propanoic acid
  SITE 5: note = (2S)-2-amino-5-carbamoylpentanoic acid
  SITE 6: note = (2S)-2-(methylamino)pentanedioic acid
  SITE 7: note = (2S)-2-amino-3-(1-methyl-1H-indol-3-yl)propanoic acid
  SITE 8: note = (2S)-2-amino-6-{[4-(carboxymethyl)piperazine-1-carb onyl]amino}hexanoic acid
  SITE 9: note = (2S)-2-amino-3-(1-methyl-1H-indol-3-yl)propanoic acid
  SITE 11: note = (2S)-2-amino-5-carbamoylpentanoic acid
  SITE 1..12: note = Optionally cyclic
  SITE 13: note = N6-(3-iodobenzoyl)-D-lysine
AANLQEWKWTQCK

SEQ ID NO: 444 - protein, 14 aa, synthetic construct
  SITE 1: note = C(O)CH2Br-(2R)-2-aminopropanoic acid
  SITE 2: note = (2S)-2-(methylamino)-3-phenylpropanoic acid
  SITE 3: note = Asparagine(trt)
  SITE 5: note = Hgl(Tert-butyl)
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 10: note = Threonine(Tert-butyl)
  SITE 11: note = Glutamic acid(Tert-butyl)
  SITE 12: note = Cysteine(Trt)
  SITE 1..12: note = Optionally cyclic
  SITE 13: note = A3-aminopropanoic acid
  SITE 14: note = Amidated D-Lysine(2-(2-(2-aminoethoxy)ethoxy)acetic acid-PEG8-Boc)
AANLQEWKWTQCAK

SEQ ID NO: 445 - protein, 14 aa, synthetic construct
  SITE 1: note = C(O)CH2Br-(2R)-2-aminopropanoic acid
  SITE 2: note = (2S)-2-(methylamino)-3-phenylpropanoic acid
  SITE 3: note = Asparagine(trt)
  SITE 5: note = Hgl(Tert-butyl)
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 10: note = Threonine(Tert-butyl)
  SITE 11: note = Glutamic acid(Tert-butyl)
  SITE 12: note = Cysteine(Trt)
  SITE 1..12: note = Optionally cyclic
  SITE 13: note = A3-aminopropanoic acid
  SITE 14: note = Amidated D-Lysine(2-(2-(2-aminoethoxy)ethoxy)acetic acid-PEG8-Boc)
AANLQEWKWTQCAK

SEQ ID NO: 446 - protein, 14 aa, synthetic construct
  SITE 1: note = C(O)CH2Br-(2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = A3-aminopropanoic acid
  SITE 14: note = Amidated D-Lysine(2-(2-(2-aminoethoxy)ethoxy)acetic acid-PEG8-NH2)
AANLQEWKWTQCAK

SEQ ID NO: 447 - protein, 14 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = A3-aminopropanoic acid
  SITE 14: note = Amidated D-Lysine(2-(2-(2-aminoethoxy)ethoxy)acetic acid-PEG8-NH2)
AANLQEWKWTQCAK

SEQ ID NO: 448 - protein, 14 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = A3-aminopropanoic acid
  SITE 14: note = Amidated D-Lysine(2-(2-(2-aminoethoxy)ethoxy)acetic acid-PEG8-2,2',2'',2'''-(1,4,7,10-tetraazacyclododecane-1, 4,7,10-tetrayl)tetraacetic acid)
AANLQEWKWTQCAK

SEQ ID NO: 449 - protein, 14 aa, synthetic construct
  SITE 1: note = (2R)-2-aminopropanoic acid
  SITE 2: note = (2S)-2-(methylamino)-3-phenylpropanoic acid
  SITE 5: note = (S)-2-aminohexanedioic acid
  SITE 6: note = (2S)-2-(methylamino)-3-phenylpropanoic acid
  SITE 7: note = (2S)-2-amino-3-(1-methyl-1H-indol-3-yl)propanoic acid
  SITE 9: note = (2S)-2-amino-3-(1-methyl-1H-indol-3-yl)propanoic acid
  SITE 1..12: note = Optionally cyclic
  SITE 13: note = A3-aminopropanoic acid
  SITE 14: note = Amidated D-Lysine(2-(2-(2-aminoethoxy)ethoxy)acetic acid-1-amino-3,6,9,12,15,18,21,24-octaoxaheptacosan-27-oic acid-2,2',2'',2'''-(1,4,7,10-tetraazacyclododecane-1,4,7,1 0-tetrayl)tetraacetic acid(Lutetium))
AANLQEWKWTQCAK